(19) United States
(12) Patent Application Publication
(10) Pub. No.: US 2007/0224644 A1
(43) Pub. Date: Sep. 27, 2007

(54) Ocular fluid markers

(76) Inventors: Lance A. Liotta, Bethesda, MD (US); Weidong Zhou, Manassas, VA (US); Virginia Espina, Rockville, MD (US); Emanuel Petricoin, Gainesville, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/698,998

(22) Filed: Jan. 29, 2007

Related U.S. Application Data
(60) Provisional application No. 60/762,499, filed on Jan. 27, 2006.

(51) Int. Cl. G01N 33/53; C12Q 1/48; G06F 19/00; A61M 31/00

(52) U.S. Cl. 435/7.1; 435/15; 604/500; 702/19

(57) ABSTRACT

The present invention relates to the analysis and monitoring of ocular fluids for determining the physiological state of an organism, to monitor drug efficacy and dynamics, for early disease detection, as well as to certain molecular markers and fingerprints of identified molecules or molecule fragments in such analysis.

### CROSS-REFERENCE TO RELATED APPLICATIONS

This application claims the benefit of earlier-filed U.S. Provisional Application Ser. No. 60/762,499, filed Jan. 27, 2006, which is incorporated herein by reference in its entirety.

### FIELD OF THE INVENTION

The present invention relates to the analysis and monitoring of ocular fluids for determining the physiological state of an organism, e.g., to monitor drug efficacy and dynamics, for early disease or other physiological state detection, as well as to monitor/quantitate certain molecular markers and fingerprints identified in such analysis.

### SUMMARY OF THE INVENTION

This invention relates to, e.g., a method of characterizing the physiological state of the eye, comprising detecting the presence or absence in vitreous fluid of one or more polypeptides, or fragments thereof; a method of characterizing the physiological state of the eye, comprising detecting the presence or absence in vitreous fluid of one or more biomarker attractant-associated polypeptides, or fragments thereof; a method of characterizing the physiological state of a living system, comprising detecting the presence or absence in a vitreous fluid of one or more polypeptides, or fragments thereof; a method of monitoring the efficacy of a tyrosine kinase inhibitor or other drug in a subject to whom said inhibitor or drug has been administered, comprising measuring the presence of a phosphorylated polypeptide in the case of said inhibitor or a polypeptide in the case of said drug in a vitreous fluid sample extracted from a subject, wherein the subject has been administered a tyrosine kinase inhibitor, drug, or a biological, chemical, protein, antibody, or other therapeutic agent.

The vitreous actively participates in the development of pathologic conditions and contains proteins that may correlate with specific retinal pathologies. These proteins have been implicated in angiogenesis, mechanical traction via increased osmolarity and aging. The proteins retained in the vitreous provide a record of the state of ocular tissues.

Vitreous fluid contains proteins that can correlate with specific retinal pathologies, such as diabetic retinopathy. Diabetic retinopathy (DR) is the most prevalent cause of vision loss in working adults. Most patients with type 1 diabetes mellitus and over 60% of those with type 2 diabetes eventually develop retinal vascular abnormalities. 20% to 30% of these patients advance to active proliferative diabetic retinopathy (PDR) and/or diabetic macular edema. Increased retinal vascular permeability (RVP) is a primary cause of diabetic macular edema and a characteristic finding in PDR. While photocoagulation surgery and vitrectomy are highly effective in reducing vision loss, early diagnosis and preventative treatments for these disorders remain a major unmet clinical need. The following discusses additional disease applications for vitreous proteomics discovery and vitreous diagnostic testing.

Wet Age-related Macular Degeneration (AMD) is the leading cause of blindness in people over the age of 55. Most severe vision loss occurs in people who develop the wet form of the disease. Treatments for and early detection of wet AMD are being developed at an unprecedented rate. Study of these new drugs has clearly established that the best chance for maintaining functional vision depends upon treatment in the earliest stages of the transition from dry to wet AMD. Furthermore, dry AMD often persists for widely varying durations (with a range of decades) prior to progressing to the wet form. For now there are only rough indicators available through subjective testing and angiography. There is a great need for a method to detect and predict the progression of AMD that could be used to augment the subjective and imaging indicators already in use.

Very little is known about the biochemical signals that modulate the exudative process that causes the severe degradation of vision in patients with wet AMD. There is some data implicating VEGF (Vascular endothelial growth factor) in this process, but almost no data regarding possible other factors. New drugs that block VEGF in the vitreous, retina and choroid have been able to slow the course of the disease but there is need for developing methods for permanently halting the progression of the disease and ultimately reversing some of its effects. Understanding the retinal proteome in wet AMD will produce opportunities for major advances in the treatment of this blinding disorder.

Retinal vein occlusion is the leading cause of vision loss after diabetic retinopathy and macular degeneration. The natural history of these diseases vary significantly. The only predictive parameter is a crude measure of retinal vascular perfusion. There is a need to stage the severity of the retinal damage and look for parameters that will predict the visual outcome. These parameters will provide improved guidance for the treating physician. In addition, these tests will be crucial in developing new treatment methods in a disease that currently has only limited treatment options.

Cystoid Macular Edema (CME) is a type of edema of the macula that causes retinal damage and occurs in a wide variety of ocular disorders. There is intense interest among practitioners and pharmaceutical companies to develop treatments for CME. Understanding the ocular fluid proteome profile of patients with CME will provide new opportunities in prevention and treatment.

Although cataracts affect millions of people per year in the USA alone, very little is know about the factors that control cataract development. Our recent findings show that lens proteins called crystallins are found in the vitreous cavity. It is also known that typical senile type cataracts form within months of removal of the gel portion (vitreous humor) of the vitreous body. Tracking the proteome of the vitreous gel and the remaining fluid that accumulates following removal of the gel may lead to the identification of new factors that could prevent cataract formation. In many ways this has been the holy grail of ophthalmology.

Tools are needed to track the pharmacokinetics of intraocular drugs delivered directly or indirectly and track ocular/systemic drug partition between vitreous and serum. Thereby drug development and modification will be greatly facilitated providing significant value to the rapidly growing industry of developing drugs for retinal disorders.

This invention provides techniques and methods which have value for such problems.

Any ocular or eye-related fluid can be analyzed in accordance with the present invention, including, e.g., vitreous fluids; aqueous fluids; retinal blood, such as blood present in the choroid; and tears, including tears extracted from the lacrimal sac. Fluids can be extracted routinely, e.g., by surgical vitrectomy procedures. In some cases the state of specific diseases as reflected in ocular fluids can be measured by fluorescent, magnetic, or radio nucleotide imaging.

The present invention provides a proteomic fingerprint of an ocular fluid sample, comprising at least one polypeptide or other molecule present in the sample. Polypeptides (also referred to as. “biomarkers”) can be isolated using any suitable technology. For example, biomarkers can be harvested from low molecular weight fractions in which a biomarker attractant is associated with a polypeptide or other biomolecules (“biomarkers”). Methods of isolating biomarker attractant-associated biomolecules are described in WO05036180, which is hereby incorporated by reference in its entirety.

The term “biomarker attractant molecule,” or “BAM,” refers to a molecule, or other substance to which biomarkers in a biological fluid adhere. In particular examples, biomarkers adhere to a BAM with a low binding affinity (for example, a binding affinity of less than 10⁻³, 10⁻⁴, 10⁻⁵, 10⁻⁶, 10⁻⁷ or 10⁻⁸ L/mol-min). An antibody may be a BAM to the extent that it binds biomarkers, other than through the specific antigen antibody interaction that results from the immune response that stimulated its production. For example, biomarker binding to an antibody BAM may occur outside of the complementarity defining region (CDR), or outside of the variable region altogether, for example by binding to the Fc portion of the antibody. However, in certain embodiments of the disclosed methods, the BAM is not an antibody. Although a particular BAM may selectively bind a class of biomarkers, the binding affinities of the biomarkers in a particular class do not differ as significantly as the binding affinities of an antigen to a particular antibody compared to other non-recognized molecules. The less specific nature of biomarker binding may be illustrated in certain examples of the BAM in which more than one biomarker binds to the BAM, for example, at least 2, at least 5, at least 10, at least 20, or even 50 or more biomarkers bind to the BAM.

Typically, BAMs have a half-life of existence in a particular biological fluid (for example in the body) that is longer than the half-life of biomarkers that become adhered to the BAMs and thereby concentrate the biomarker in the biological fluid. For example, BAMs can have a half-life of greater than about 1 day, such as greater than 2, 5, 10, 20 or 50 days. In particular examples, the BAM has size and/or shape such that it is not substantially filtered from the blood by the kidneys. In other particular examples, the BAM has a molecular weight of greater than 25 kDa, for example, greater than 30, 50, 75, 100, 150, 200 or 300 kDa. In yet other particular examples, the BAM molecule has a molecular weight falling within a particular range, for example between 30 and 50 kDa, between 50 and 75 kDa, between 75 and 100 kDa, between 100 and 150 kDa, between 150 and 200 kDa, between 200 and 300 kDa, or any other range between 30 kDa and 300 kDa. Biomarkers may adsorb to the surface or be absorbed into the interior of the BAM, or both.

Examples of BAMs include proteins (including natural and engineered proteins such as chimeric proteins, proteins with modified amino acid composition, proteins modified postranslationally, nucleic acids, carbohydrate decorated molecules, and organic polymers), dendrimers and particles (such as microparticles and nanoparticles, including silica, metal, ceramic and carbohydrate microparticles and nanoparticles), and cellular microparticles (see, for example, Diamant et al, Eur J Clin Invest. 34: 392-401, 2004).

BAMs may be produced or derivatized to provide ionic groups (such as carboxylate, protonated amine, quaternary ammonium, and sulfate groups), hydrogen-bond acceptors or hydrogen-bond donors, electron donors or electron acceptors, polar groups (such as amino, hydroxyl, ester, sulfhydryl and nitrile groups), hydrophobic groups (such as alkyl, alkenyl and alkynyl groups or groups with specific partition coefficients), peptides, proteins, nucleic acids, carbohydrates, lipids or any combination thereof, on their surfaces or in their interiors. Where the BAM is a protein, such as a naturally occurring protein, it may also be referred to as a “carrier protein” to reflect its role in collecting and concentrating LMM biomarkers from biological fluids. Examples of carrier proteins include albumin, iron binding proteins (such as transferrin), fibrinogen, alpha-2-macroglobulin, immunoglobulins (such as IgA, IgE and IgG), complement, haptoglobulin, lipoproteins, prealbumin, alpha-l-acid glycoprotein, fibronectin, and ceruloplasmin, and fragments, combinations and chemical derivatives thereof.

A proteomic fingerprint can comprise as few as one polypeptide, or it can comprise more than one polypeptide (i.e., a plurality). Any method of analyzing ocular fluid content can be utilized. For example, biomarker attractants (also referred to as a biomarker attractant molecules or “BAM”) present in an ocular fluid can be utilized without limitation for purification purposes, including albumin, proteoglycans, glucosaminglycans, and heparan sulfates. The polypeptides can be present as intact proteins, or as fragments. Such fragments can be naturally-occurring, or can be produced during processing of a sample, either by inadvertent or deliberate proteolysis (e.g., contacting a sample with a proteolytic enzyme or a chemical cleavage agent).

Examples of biomarkers that have been isolated from ocular fluids are shown in Table 2 to Table 13. These were obtained by running a sample of ocular fluid on an SDS-PAGE gel, and then digesting the entire gel lane from high to low protein molecular weight with trypsin, followed by MS/MS analysis.

The set of polypeptides detected in accordance with the present invention can be described as a “fingerprint” in that they are a distinctive pattern of polypeptides present in the ocular fluid. Fingerprints can be prepared using the BAM techniques described above, or using other technologies or purification processes, e.g., characterizing polypeptides present in the ocular fluids without a BAM-enrichment step (See Table 2 to Table 13 for a representative example of such polypeptides).

Just as with a fingerprint, the set of polypeptides can be used as a unique identifier to characterize the fluid, as well as the physiological status of the subject. The ocular fingerprint can be viewed as a snapshot of the elements (e.g., polypeptides) that are involved in, or a product of, the physiological processes that are occurring in the body. Examples of physiological states that can be characterized in accordance with the present invention include without limitation, diseases states (e.g., cancer, retinopathy, diabetes, macular degeneration, venous occlusive disease, cataracts, and other disorders mentioned herein); therapeutic states (e.g., for monitoring drug efficacy and adverse events); organ function (e.g., to monitor normal organ function, such as brain, kidney, and liver functions); toxicological states (e.g., to detect toxins or perturbations caused by toxins); etc. Thus, an ocular fluid fingerprint can be used for a variety of medical, diagnostic, and therapeutic purposes, including, for example: to detect the risk of cataract formation (see below); to monitor blood-ocular breakdown; to detect age-related macular degeneration; to detect therapeutic efficacy of kinase inhibitors and other drugs; etc.

For example, ocular fluids can be removed from a patient using a whole-bore vitrectomy cannula or cutter containing agents that inhibit polypeptide degradation, and then subjecting the fluid to analysis for the presence of biomarkers. Such biomarkers can be used to determine the risk of cataracts (e.g., when crystallins are elevated); the integrity of the blood-ocular barrier; and other retinal conditions and diseases. This can be especially useful in patients who are at risk for an ocular disease, e.g., subjects with diabetes, aging subjects, or subjects who have been identified as a carrier of a gene defect associated with an ocular disorder.

Other viable polypeptide candidates which can be routinely analyzed for the assessment of the physiological state of an individual comprise proteins that are pathologically correlated with certain physiological state. For example, retinol binding protein-4 (RBP4) is known to contribute to the development of diabetes by blocking the action of insulin. Ocular detection of this protein may lead to important insights into the initiation and/or progression of diabetes in a subject. Other examples of these polypeptide candidates include, but are not limited to, Secreted Protein Acidic and Rich in Cysteine (SPARC). It is known that SPARC is upregulated after injury and modulates cell adhesion and proliferation and by releasing the KGHK peptide, which stimulates angiogenesis. SPARC binds VEGF, inhibits its interaction with extracellular surface, and also inhibits activation of downstream effectors (e.g., ERK1/2) and VEGF-induced DNA synthesis. It has been thought that SPARC not only modulates angiogenesis but, moreover, regulation of SPARC levels appears to be the key to control angiogenesis in macular degeneration. A third example of a protein that may be utilized as a diagnostic marker of a disease state (for example, cancer) comprises detection of phosphorylation status of Akt. Akt is activated by growth factors or cytokines in a PI3K-dependent manner, and phosphorylation of two residues by PDK1 (T308) and PDK2 (S473) is required for its full activation. The instant method comprises detecting the phosphorylation status of one or more amino acid residues of Akt in normal subject and a patient, and comparing the status with, for example, progression of cancer in the patient. Other cancer biomarkers, for e.g., VEGFR, EGFR, Bcr-Abl, Her2-Neu (erbB2), TGFR, etc. may also be routinely analyzed.

Therefore, in addition to the ocular disorders, the ocular fluids can also be used generally to monitor a subject's health and physiological status. The ocular fluid is in communication with other body compartments, and thus is useful to monitor extra-ocular compartments, including the brain, kidney, liver, etc. Since developmentally the eye is an extension of the brain the state of the molecular composition of ocular fluids can provide information about diseases in the brain. With regard to more distant organs, molecules derived from these organs may enter the ocular fluids through the circulation, or the ocular fluid markers may reflect a systemic body-wide process that effects the distant organ.

The present invention also relates to methods of monitoring the physiological status of a subject, comprising: measuring the presence of a post-translationally modified polypeptide (e.g., phosphorylation) in a vitreous fluid sample extracted from a subject. In certain claims, signaling pathways can be monitored. Signaling pathways include any pathway in the body that involves generating a chemical event (e.g., phosphorylation) that modulates a cellular activity (e.g., indicating receptor occupancy, site-directed protein-protein binding, and triggering a cascade of enzymatic reactions that culminates in gene expression). For example, phosphorylation is a key post-translational modification event in many biological pathways involved in cell growth, cell death, gene expression, and cellular responses to stimuli. In addition, aberrant phosphorylation patterns may be associated with diseases, such as cancer and other hyper-proliferation disorders.

Further examples include, e.g., G-protein receptor mediated pathways, especially receptors for tyrosine kinases, such as vascular growth factor receptors (e.g., VEGFR-1, VEGFR-2), epidermal growth factor receptors (EGFR), HER2, adrenergic receptors (e.g., alpha- and beta-types); hormone mediated receptors; etc. Examples of receptors include, VEGFR-2 (e.g., including phosphorylation sites Y951, Y996, Y1054, Y1059, Y1175, Y1214); PDGFR-beta (e.g., including phosphorylation sites Y740, Y751, and Y771), and EGFR (e.g., including phosphorylation sites Y1173, Y1148, Y1068, Y845, and Y992).

These methods can also be utilized to measure or monitor the efficacy of a drug, especially a drug which is utilized to modulate a kinase, such as a tyrosine kinase, or a biological based therapeutic, such as an autologous platelet concentrate. A variety of therapeutic agents are being used to treat diseases or disorders associated with aberrant or increased kinase activity, including cancers and angiogenesis. Targets include, but are not limited to, e.g., raf, PDGFR-alpha, PDGFR-beta, EGFR, VEGFR, VEGFR1, VEGFR2, VEGFR3, HER-2, KIT, FLT3, c-MET, FGFR, FGFR1, FGFR3, c-FMS, RET, ABL, ALK, ARG, NTRK1, NTRK3, JAK2, ROS, etc. Other signaling targets include, e.g., ERK, AKT, PYK2, etc.

Examples of kinase effecting drugs, include, but are not limited to, e.g., avastin (bevacizumab), cetuximab, erlotinib (tarceva or OSI774), everolimus (RAD0001), fasudil, FK506, gefitinib (ZD1839), imatinib mesylate (STI57 or Gleevec), lapatinib ditosylate (GSK572016), rapamycin, sorafinib, sirolimus, sunitinib (sutent), trastuzumab (Herceptin), serafanib and wortmannin.

One goal of such drug therapy is to reduce the amount of phosphorylation of a target polypeptide. For example, several anti-cancer drugs are being utilized to block angiogenesis by blocking the phosphorylation of VEGFR-2. The efficacy of such drugs can be monitored by detecting the appearance of shed phosphorylated receptor into the vitreous fluid. As shown in the attached example, phosphorylated VEGFR-2 and PDGF-R polypeptide fragments were detected in vitreous fluid using reverse phase assays.

Reverse phase protein microarray is a technique that is routinely used for efficient and accurate detection of proteins in a sample. The proteins extracted from a single sample are immobilized on the substratum. The captured analytes are detected with a primary antibody directed toward the protein/polypeptide of interest and a second tagged molecule is incorporated for the detection strategy. Each spot on the array corresponds to a different sample. Total lysates of different samples are immobilized on the array and incubated with one antibody. Each spot on the array corresponds to a different sample (up to 640 lysates per array). Reverse phase microarray allows for probing into the networking and cross-talk between proteins involved in intracellular signaling. Uses of reverse phase microarray techniques in, for example, microarray printing, protein detection, and/or protein quantification are all commensurate with the scope of the instant invention.

Detection of polypeptides can be made by any suitable technique. Polypeptide backbone can be detected, as well as post-translational modifications of it, such as glycosylation and phosphorylation. Antibodies can be used routinely, e.g., which are generated to amino acid epitopes of the target polypeptide; phosphorylated amino acids, etc. Reverse phase assay can be used to detect ocular polypeptides, where the array is comprised of ocular fluid immobilized to a substrate such as nitrocellulose, and binding partners (such as antibodies) are applied that specifically bind the target of interest. These can be rapidly used to characterize the contents of the fluid and generate disease biomarkers, including proteomic fingerprints. See, e.g., Grubb et al., Proteomics, 3:2142-2146, 2003. Mass spectroscopy and other conventional proteomic methods can also be used.

In the instant invention, there is provided a method for detecting macular diseases, retinal detachment, inflammation of the eye, diabetic retinopathy and many other diseases comprising comparing a profile of shed receptors or signal transduction molecules and/or their phosphorylated forms, for e.g., VEGFR, PDGFR, EGFR, RBP4 in a healthy subject with that of a patient. Of note is that these receptor proteins are known to be existing drug targets for existing drugs such as Gleevac, Iressa, and Avastin, demonstrating that this information could be used to tailor therapy for the patient. With regard to cataracts the instant invention relates to identification of a series of crystallins in vitreous samples of patients who have had a vitrectomy for retinal detachment. Such patients have virtually a certain chance of immediately developing cataracts. With regard to macular hole or macular detachment therapy or macular vascular leak, therapy can include administration of natural autologous protein such as platelet extracts.

Given the correlations described in this application, for a particular patient, the presence of a disease will be measured by, for example, extracting vitreous fluid from the patient and determining the content of the particular polypeptide or fragment of interest using fully conventional methods such as (immunologic techniques, antibody diagnostics, radioimmunoassays, mass spectrometry, microarrays, western blotting, gel electrophoresis, and labeled or enzyme amplified diagnostic technologies.

Using the technique described previously, one of ordinary skill in the art, using routine methods may develop correlations that cater to, or are specific towards the detecting and diagnosis of a particular disease. For instance, the method provides a means for characterizing the identity and/or content of vitreous fluid with respect to the levels or amounts of particular peptides which will be indicative of disease. Peptides that are unique to a disease, wherein the presence of any amount of such peptides will indicate the likelihood of the disease being present are described. One of ordinary skill in the art could utilize existing knowledge of peptide biomarkers which correlate with a particular disease or a physiological state, and screen for said peptide(s) using the method described by the instant invention. In some cases the presence or absence of the molecule above background may be diagnostic of the disease, because that molecule may not be expected otherwise. An example is molecules associated with vascular leakage during wet macular degeneration. In other cases the level of the molecule concentration or the level of the phosphorylated molecules (phosphorylation on one or more specific residues) may be quantitatively related to the severity of the disease or the amount of disease suppression produced by a drug administered to the patient. An example is a method for detecting the phosphorylation status of the VEGFR, (which may have no correlation with the amount of total receptor protein) as a predictor of (a) requirement for an angiogenesis inhibitor, and (b) whether or not an angiogenesis inhibitor is working to suppress the VEGF ligand from triggering its receptor. If the receptor is active or engaged with ligand then and only then will it be phosphorylated.

The instant invention relates to the use of the vitreous fluid as a reservoir of important biological markers. As is explained in the tables, samples may be isolated from a live specimen or from a cadaver. Tables 2-13 provide a representative list of peptides which are present in the vitreous fluid of the eye.

The instant invention also provides a method for identification of novel proteins/peptides which are potential biomarkers of diseases and/or physiological state in subject. Representative examples of such peptides in relation to ocular diseases (for e.g., macular hole, retinal degeneration, or a combination of macular hole and retinal degeneration) are provided in the tables (Tables 5-13). Polypeptides that are specifically associated with a disease, for e.g., when compared to a different disease or a control sample, are highlighted/underlined.

Additionally, the present invention relates to a method for detecting proteins in the vitreous fluid of the eye comprising isolation of the protein, enzymatic hydrolysis (for e.g., using trypsin), HPLC separation, resolved using mass spectrometric analysis, and the retrieved fragments are searched a database of candidate polypeptides. Routine methods for HPLC analysis of peptides are known in the art, and may involve utilization of separation columns and/or buffers of interest (for e.g., modified C-18 column). Techniques for mass-spectrometric analysis of peptides are also known, and may involve, for e.g., nano-spray/linear Ion Trap mass spectrometric analysis.

The present invention also provides an improved hollow bore cannula or cutter for performing a vitrectomy, wherein the improvement comprises a reservoir in said cannula or cutter that comprises at least one chemical to protect polypeptide integrity. Chemicals that can be included in the reservoir include, e.g., protease inhibitors; phosphatase inhibitors; etc. Specific examples include, serine protease inhibitors, cysteine protease inhibitors, aspartic protease inhibitors, and metalloprotease inhibitors. Examples of these include, AEBSF, aprotinin, E-64, EDTA, leupeptin, bestatin, O-phenanthroline, cathepsin, etc.

Without further elaboration, it is believed that one skilled in the art can, using the preceding description, utilize the following invention to its fullest extent. The following specific preferred claims are, therefore, to be construed as merely illustrative, and not limitative of the remainder of the disclosure in any way whatsoever.

The entire disclosures of all applications, patents and publications, cited above and below, are hereby incorporated by reference.

### BRIEF DESCRIPTION OF THE FIGURES

FIG. 1 depicts the validation of ocular markers in one microliter of vitreous fluid obtained from a living patient donor. The amplitude of the bars is the relative density of the amplified antibody signal relative to total protein in the sample for the designated analyte. Based on dilution curves, calibrators, and controls it can be readily assessed that the concentration of the molecule being measured is above background and within the linear range of the assay.

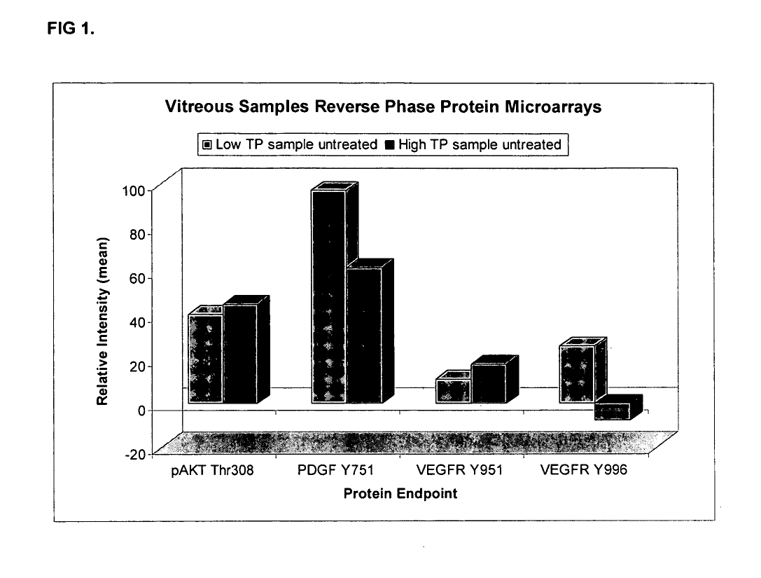

FIG. 2 shows a dilution curve for each analyte verifying that the assay is linear over the detection range. These data conclusively validate the existence of these identified molecules and demonstrate that they can be measured in very small volumes that can be sampled by a microneedle in a routine clinical setting. All antibodies used fo detection were tested to insure antigen specificity. The antibodies detected phosphorylated residues on the analyte protein recognize the protein only if it phosphorylated on the specific residue. If the protein is not phosphorylated on this residue or is phosphorylated on another residue, then the antibody will not bind and the value is zero.

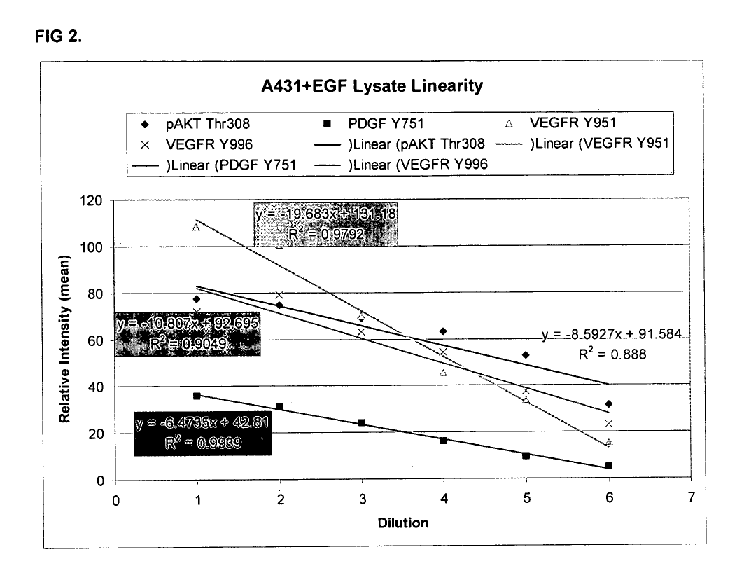

The invention will be explained below with reference to the following non-limiting examples.

### EXAMPLES

In the forgoing and in the following examples, all temperatures are set forth uncorrected in degrees Celsius (° C.) and, all parts and percentages are by weight, unless otherwise indicated.

Experimental Procedure

Vitreous Sampling:

Pars Plana Vitrectomy

All vitreous samples were obtained prior to the vitrectomy portion of the surgery. The surgery would have been done regardless of participation in the study. The patient was prepped and draped in the usual sterile fashion. Prior to the vitrectomy portion of the study, a minute amount of vitreous (approximately 0.1 ml) was obtained in a sterile TB syringe through the pars plana. The vitreous sample was then frozen at −20° C. or −80° C. for storage and subsequent analysis of the vitreous proteome. There was no additional risk to the patient in addition to that incurred from the surgery alone.

Autopsy Study

At an unrestricted consented autopsy, 2-8 ml of clear vitreous gel were extracted by inserting in the lateral palpebral fissure a 22 g needle attached to a 10 ml tuberculin. The samples were immediately frozen at −80° C.

Approximately 5-10 cc of vitreous was collected from each eye. Nine separate autopsy samples were procured. The samples were stored at −80° C. until the analysis could be performed.

Samples From Patients Undergoing a Total Vitrectomy

The patients were prepared accordingly to the anesthetic/surgical protocols prescribed for the specific pathology which the patients were suffering from. Vitrectomy was carried out using a surgical microscope and external lenses designed to provide a clear image of the back of the eye. Using the sclerotome, tree tiny incisions just a few millimeters in length were made on the sclera, then the retinal surgeon inserted the following instruments: 1) a fiber optic light source to illuminate inside the eye; 2) the infusion line to maintain the eye's shape and tone during surgery and 3) the vitrectome to cut and remove the vitreous. The total vitreous was aspirated by the vitrectome and diluted 5-8 times with Ringer-lactate buffer solution kept at room temperature during the surgical session, depending on the length of the surgical procedure, on whether additional procedures were required and on the overall health of the eye. Immediately after the vitreous removal, the cassette containing the diluted vitreous was placed at 4° C., and within 60 min gently aspirated, then diluted 1:1 with cold Ringer-lactate buffer solution (at 4° C.). Then the suspension was carefully mixed five times, then passed through a sterile pipette with narrow tip (20 passages) until any macroscopic material was completely dissolved. Finally, the re-suspended material was divided into small aliquots (1 ml) into plastic tubes previously labeled, immediately frozen with liquid nitrogen and stored at −80° C. within 15 min. Smaller aliquots were also frozen to carry out subsequently the protein quantification, using a commercial Bradford assay (Biorad). All the procedures were carried out using sterile plastic ware.

To develop a reproducible procedure to analyze the proteins contained in eye tissues, preliminary experiments were carried out with bovine eyeballs obtained from a local slaughterhouse. The eyeballs were maintained at 4° C. until the vitreous was extracted, i.e. 6-8 hours after the death of the animal. The eyeballs were cut 3 mm posterior to the limbus and the whole vitreous and the whole lens were removed as described (Facchiano et al, 1996). Then, samples were re-suspended by mechanical dissociation of the material using cold saline buffer. Protein concentration and stability in the eye tissues homogenates was checked by SDS-PAGE analysis and silver staining procedure.

Nanoflow Reversed-Phase Liquid Chromatography Tandem Mass Spectrometry

Vitreous samples were digested by trypsin and peptides were purified by Zip-tip (Waters). The peptides were then analyzed by reversed-phase liquid chromatography nanospray tandem mass spectrometry using a linear ion-trap mass spectrometer (LTQ, ThermoElectron, San Jose, Calif.). Reverse phase column was slurry-packed in-house with 5 μm, 200 Å pore size C₁₈ resin (Michrom BioResources, CA) in 100 μm i.d.×10 cm long fused silica capillary (Polymicro Technologies, Phoenix, Ariz.) with a laser-pulled tip. After sample injection, the column was washed for 5 min with mobile phase A (0.1% formic acid) and peptides were eluted using a linear gradient of 0% mobile phase B (0.1% formic acid, 80% acetonitrile) to 50% mobile phase B in 30 min at 250 nl/min, then to 100% B in an additional 5 min. The LTQ mass spectrometer was operated in a data-dependent mode in which each full MS scan was followed by five MS/MS scans where the five most abundant molecular ions were dynamically selected and fragmented by collision-induced dissociation (CID) using a normalized collision energy of 35%.

Bioinformatic Analysis

Tandem mass spectra were matched against Swiss-Prot human database through the Sequest Bioworks Browser (ThermoFinnigan) using tryptic cleavage constraints and static cysteine alkylation by iodoacetamide. For a peptide to be considered legitimately identified, it had to achieve cross correlation scores of 1.5 for [M+H]¹⁺, 2.0 for [M+2H]²⁺, 2.5 for [M+3H]³⁺, ΔCn>0.1, and a maximum probabilities of randomized identification of 0.01.

TABLE 1
Vitreous demographic information (S: Sex; PM: Post mortem)

| Vitreous | | | | PM | Vitreous | Vitreous |
|---|---|---|---|---|---|---|
| samples | Age | S | Cause of Death/Diagnosis | Interval | Color | Claritiy |
| 2005-35 | 35 | M | Peritonitis/Nephropathic cytinosis | 72 h | Pale Pink | Clear |
| 2005-41 | 41 | M | Disseminated Aspergillus/GBM | 12 h | Colorless | Clear |
| 2005-49 | 59 | M | Metastatic Renal cell carcinoma | 5 h | Colorless | Clear |
| 2005-52 | 59 | F | Undetermined/AML | 14 h | Colorless | Clear |
| 2005-56 | 71 | M | Pneumonia/Multiple Sclerosis | 20.5 h | Colorless | Clear |
| 2005-60 | 40 | F | Pneumonia/ALL | 62 h | Pale Pink | Clear |
| 2005-67 | 20 | M | Pneumonia/Alexander's Disease | 8 h | Colorless | Clear |
| 2005-71 | 24 | M | CVA/Chronic Granulomatous Disease | 33 h | Colorless | Clear |
| 2005-78 | 64 | M | Cirrhosis/Hepatitis C | 48 h | Yellow | Clear |
| 2004-34 | 16 | M | Neurotoxoplasmosis/Myelodysplasia | 16 h | Colorless | Clear |
| 2004-39 | 53 | M | CVA/Hairy Cell Leukemia | 8.5 h | Colorless | Clear |
| 2005-74 | 46 | F | dad/aml-m5 | 5 h | Colorless | Clear |

TABLE 2
Osteopontin precursor
Interleukin precursors
Neural cadherin precursor
PROSAAS precursor (Granin-like neuroendocrine peptide precursor)
Interphotoreceptor retinoid-binding protein precursor
Dickkopf related protein-3 precursor
TriadinMutS protein homolog 4
Wnt inhibitory factor 1 precursor
S-arrestin
Von Ebner's gland protein precursor
Opticin
Cofilin
Receptor precursors
TCP11b protein
Kidney-specific membrane protein NX-17
Retinoic Acid Receptor Responder Protein
Retinoblastoma-associated factor 600
Retinoschisin
Muscarinic acetylcholine receptor M5
Phosphatidylethanolamine-binding Protein
BAG-family molecular chaperone regulator-1
Beta crystallin B

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

It is believed that one skilled in the art, using the preceding information and information available in the art, can utilize the present invention to its fullest extent. It should be apparent to one of ordinary skill in the art that changes and modifications can be made to this invention without departing from the spirit or scope of the invention as it is set forth herein. The topic headings set forth above and below are meant as guidance where certain information can be found in the application, but are not intended to be the only source in the application where information on such topic can be found. All publications and patents cited above are incorporated herein by reference.

TABLE 3.1
Proteins identified in vitreous fluids (I)

| | | P | Accession | Id | Id No | Pat |
|---|---|---|---|---|---|---|
| | Protein Name | (probability) | No | Pep | on 19 | No |
| 1 | albumin precursor; PRO0883 protein [Homo sapiens] | 1.00E−30 | 4502027 | 95 | 19 | 11 |
| 2 | transthyretin; prealbumin [Homo sapiens] | 1.00E−30 | 4507725 | 23 | 19 | 11 |
| 3 | transferrin; PRO2086 protein [Homo sapiens] | 1.11E−15 | 4557871 | 52 | 19 | 11 |
| 4 | serine (or cysteine) proteinase inhibitor, clade A (alpha-1 antiprotei | 1.11E−16 | 21361198 | 16 | 19 | 11 |
| 5 | clusterin isoform 1; complement-associated protein SP-40 [Homo sapiens | 1.53E−13 | 42716297 | 14 | 19 | 11 |
| 6 | complement component 3 precursor; acylation-stimulating protein cleavag [symbol] | 8.55E−14 | 4557385 | 10 | 18 | 10 |
| 7 | ceruloplasmin (ferroxidase); Ceruloplasmin [Homo sapiens] | 2.96E−12 | 4557485 | 8 | 18 | 11 |
| 8 | hemopexin [Homo sapiens] | 6.22E−14 | 11321561 | 9 | 18 | 10 |
| 9 | serine (or cysteine) proteinase inhibitor, clade F (alpha-2 antiplasmi | 1.05E−12 | 39725934 | 13 | 18 | 11 |
| 10 | orosomucoid 2; alpha-1-acid glycoprotein, type 2 [Homo sapiens] | 2.73E−13 | 4505529 | 5 | 17 | 10 |
| 11 | vitamin D-binding protein precursor; vitamin D-binding alpha-globulin | 1.49E−13 | 32483410 | 7 | 17 | 9 |
| 12 | complement component 4B proprotein [Homo sapiens] | 3.11E−14 | 4502501 | 6 | 16 | 11 |
| 13 | apolipoprotein A-I precursor [Homo sapiens] | 4.91E−12 | 4557321 | 10 | 16 | 10 |
| 14 | orosomucoid 1 precursor; alpha-1-acid glycoprotein 1; Orosomucoid-1 (al [symbol] | 5.08E−13 | 9257232 | 6 | 15 | 10 |
| 15 | RBP3 gene product [Homo sapiens] | 3.33E−15 | 4506453 | 16 | 14 | 10 |
| 16 | beta-2-microglobulin precursor [Homo sapiens] | 9.99E−15 | 4757826 | 3 | 14 | 10 |
| 17 | gelsolin isoform b [Homo sapiens] | 1.00E−30 | 38044288 | 4 | 14 | 9 |
| 18 | apolipoprotein E precursor; apolipoprotein E3 [Homo sapiens] | 1.58E−11 | 4557325 | 6 | 13 | 9 |
| 19 | EGF-containing fibulin-like extracellular matrix protein 1 isoform b; f | 1.03E−11 | 9665253 | 3 | 13 | 9 |
| 20 | PREDICTED: hypothetical protein XP_373740 [Homo sapiens] | 3.65E−05 | 41222847 | 2 | 13 | 9 |
| 21 | complement factor B preproprotein; C3 proactivator; C3 proaccelerator; | 2.63E−09 | 4502397 | 4 | 12 | 5 |
| 22 | complement component 1 inhibitor precursor [Homo sapiens] | 1.28E−09 | 4557379 | 4 | 12 | 10 |
| 23 | supervillin isoform 2; membrane-associated F-actin binding protein p20 | 1.51E−03 | 11496982 | 1 | 12 | 9 |
| 24 | apolipoprotein A-II precursor [Homo sapiens] | 1.60E−11 | 4502149 | 4 | 11 | 6 |
| 25 | cystatin C precursor; cystatin 3; gamma-trace; post | 1.15E−10 | 4503107 | 6 | 11 | 9 |
| 26 | alpha-1-antichymotrypsin, precursor; alpha-1-antichymotrypsin; antichym | 3.02E−09 | 4501843 | 3 | 10 | 7 |
| 27 | alpha-2-macroglobulin precursor [Homo sapiens] | 5.16E−12 | 4557225 | 8 | 10 | 10 |
| 28 | beta-2-glycoprotein I precursor [Homo sapiens] | 5.22E−14 | 4557327 | 4 | 10 | 5 |
| 29 | prostaglandin D2 synthase 21 kDa; prostaglandin D synthase; prostagland [symbol] | 5.88E−14 | 32171249 | 3 | 10 | 8 |
| 30 | dickkopf homolog 3; RIG-like 7-1; RIG-like 5-6; dickkopf (Xenopus laev | 7.77E−15 | 40548389 | 3 | 10 | 9 |
| 31 | kininogen 1; alpha-2-thiol proteinase inhibitor; bradykinin [Homo sapie | 1.79E−08 | 4504893 | 3 | 9 | 5 |
| 32 | secreted phosphoprotein 1 (osteopontin, bone sialo | 3.44E−14 | 4759166 | 5 | 9 | 9 |
| 33 | haptoglobin [Homo sapiens] | 7.34E−11 | 4826762 | 7 | 9 | 6 |
| 34 | plasma glutathione peroxidase 3 precursor [Homo sapiens] | 1.54E−11 | 6006001 | 2 | 9 | 7 |
| 35 | haptoglobin-related protein; Haptoglobin-related locus [Homo sapiens] | 5.75E−09 | 45580723 | 7 | 9 | 6 |
| 36 | alpha-1-microglobulin/bikunin precursor; Alpha-1-microglobulin/bikunin | 8.33E−11 | 4502067 | 3 | 8 | 8 |
| 37 | serine (or cysteine) proteinase inhibitor, clade C (antithrombin), memb | 3.24E−07 | 4502261 | 3 | 8 | 6 |
| 38 | crystallin, beta B1; eye lens structural protein [Homo sapiens] | 1.56E−11 | 4503061 | 22 | 8 | 3 |
| 39 | crystallin, beta B2; eye lens structural protein [Homo sapiens] | 4.24E−11 | 4503063 | 7 | 8 | 4 |
| 40 | cathepsin D preproprotein [Homo sapiens] | 2.95E−10 | 4503143 | 2 | 8 | 7 |
| 41 | I factor (complement) [Homo sapiens] | 1.88E−07 | 4504579 | 2 | 8 | 6 |
| 42 | RBP4 gene product [Homo sapiens] | 9.77E−14 | 5803139 | 3 | 8 | 6 |
| 43 | alpha 1B-glycoprotein [Homo sapiens] | 2.60E−12 | 21071030 | 2 | 8 | 6 |
| 44 | transcription factor-like protein 4 isoform gamma; MAX-like bHLHZIP pr | 2.12E−03 | 24586667 | 1 | 8 | 5 |
| 45 | tripeptidyl-peptidase I precursor [Homo sapiens] | 1.00E−30 | 5729770 | 1 | 7 | 7 |
| 46 | opticin; opticin, oculoglycan; oculoglycan [Homo sapiens] | 3.15E−09 | 7657419 | 2 | 7 | 4 |
| 47 | crystallin, beta A3; eye lens structural protein [Homo sapiens] | 2.22E−15 | 12056461 | 11 | 7 | 2 |
| 48 | apolipoprotein A-IV precursor [Homo sapiens] | 4.20E−11 | 4502151 | 4 | 6 | 3 |
| 49 | crystallin, alpha A; crystallin, alpha-1; human alphaA-crystallin (CRYA | 6.66E−15 | 4503055 | 9 | 6 | 2 |
| 50 | crystallin, alpha B; heat-shock 20 kD like-protein [Homo sapiens] | 1.82E−11 | 4503057 | 6 | 6 | 2 |
| 51 | lipocalin 1 precursor; lipocalin 1 (protein migrati | 1.23E−12 | 4504963 | 4 | 6 | 6 |
| 52 | angiotensinogen precursor; angiotensin II precursor; pre-angiotensinoge | 2.70E−12 | 4557287 | 4 | 6 | 6 |
| 53 | lysozyme precursor [Homo sapiens] | 9.99E−15 | 4557894 | 5 | 6 | 6 |
| 54 | phosphodiesterase 6A, alpha subunit [Homo sapiens] | 2.08E−04 | 4585864 | 1 | 6 | 5 |
| 55 | GTPase regulator associated with the focal adhesion kinase pp125; GTPas | 1.08E−03 | 7662208 | 1 | 6 | 4 |
| 56 | crystallin, gamma S; crystallin, gamma 8 [Homo sapiens] | 8.39E−13 | 8922120 | 12 | 6 | 3 |
| 57 | amyloid beta (A4) precursor-like protein 2; amyloi | 1.07E−09 | 4502147 | 3 | 5 | 5 |
| 58 | carboxypeptidase E precursor [Homo sapiens] | 7.12E−12 | 4503009 | 4 | 5 | 5 |
| 59 | beta globin; hemoglobin beta chain [Homo sapiens] | 9.99E−15 | 4504349 | 6 | 5 | 4 |
| 60 | histidine-rich glycoprotein precursor; histidine-proline rich glycoprot | 5.05E−08 | 4504489 | 2 | 5 | 4 |
| 61 | amyloid beta (A4) precursor-like protein 1 [Homo sa | 5.89E−08 | 4885065 | 1 | 5 | 5 |
| 62 | glyceraldehyde-3-phosphate dehydrogenase [Homo sapiens] | 5.99E−10 | 7669492 | 3 | 5 | 5 |
| 63 | dual specificity phosphatase 5; serine/threonine specific protein phos | 1.12E−04 | 12707566 | 1 | 5 | 4 |
| 64 | ectonucleotide pyrophosphatase/phosphodiesterase 2 | 2.66E−14 | 20070230 | 2 | 5 | 5 |
| 65 | hypothetical protein DKFZp434O0527 [Homo sapiens] | 7.81E−04 | 34916026 | 1 | 5 | 4 |
| 66 | afamin precursor; alpha-albumin [Homo sapiens] | 1.10E−12 | 4501987 | 1 | 4 | 3 |
| 67 | lysosomal trafficking regulator; beige protein [Hom | 5.78E−05 | 4502839 | 1 | 4 | 4 |
| 68 | chitinase 3-like 1; cartilage glycoprotein-39 [Homo sapiens] | 9.78E−06 | 4557018 | 1 | 4 | 3 |
| 69 | skeletal muscle specific actinin, alpha 3 [Homo sapiens] | 3.34E−04 | 4557241 | 1 | 4 | 3 |
| 70 | Niemann-Pick disease, type C2 precursor; epididymal | 1.08E−10 | 5453678 | 1 | 4 | 4 |
| 71 | Wnt inhibitory factor-1 precursor; Wnt inhibitory factor-1 [Homo sapien | 2.09E−08 | 6005950 | 3 | 4 | 4 |
| 72 | hypothetical protein FLJ20580 [Homo sapiens] | 8.20E−04 | 8923541 | 1 | 4 | 3 |
| 73 | arginyl-tRNA synthetase [Homo sapiens] | 2.46E−03 | 15149476 | 1 | 4 | 4 |
| 74 | enolase 3; enolase-3, beta, muscle; muscle specif | 8.88E−15 | 16554592 | 1 | 4 | 4 |
| 75 | ribosomal protein L10-like protein [Homo sapiens] | 1.50E−03 | 18152783 | 1 | 4 | 2 |
| 76 | SPARC-like 1; hevin; mast9 [Homo sapiens] | 9.96E−08 | 21359871 | 1 | 4 | 4 |
| 77 | mutS homolog 5 isoform a; mutS (E. coli) homolog 5 [Homo sapiens] | 6.18E−03 | 26638662 | 1 | 4 | 4 |
| 78 | dual oxidase 2 precursor; dual oxidase-like domains 2; nicotinamide ad | 2.36E−04 | 28872753 | 1 | 4 | 4 |
| 79 | pancreatic ribonuclease precursor; RNase upl-1 [Homo sapiens] | 1.24E−08 | 38201682 | 1 | 4 | 4 |
| 80 | FLT25005 protein [Homo sapiens] | 4.55E−03 | 38570111 | 1 | 4 | 4 |
| 81 | PREDICTED: hypothetical protein XP_376099 [Homo sapiens] | 6.72E−05 | 42656388 | 1 | 4 | 3 |
| 82 | PREDICTED: KIAA1414 protein [Homo sapiens] | 2.04E−03 | 51460558 | 1 | 4 | 3 |
| 83 | PREDICTED: cardiomyopathy associated 3 [Homo sapiens] | 5.89E−05 | 51460893 | 1 | 4 | 2 |
| 84 | PREDICTED: hypothetical protein FLJ25756 [Homo sapiens] | 4.76E−04 | 51472397 | 2 | 4 | 4 |
| 85 | PREDICTED: KIAA0179 [Homo sapiens] | 8.94E−04 | 51475296 | 1 | 4 | 4 |
| 86 | PREDICTED: similar to phosducin-like 3; phosducin-like 2; IAP-associat | 5.29E−05 | 51492730 | 3 | 4 | 2 |
| 87 | alpha 1 actin precursor; alpha skeletal muscle act | 1.29E−05 | 4501881 | 1 | 3 | 3 |
| 88 | alpha-2-HS-glycoprotein; Alpha-2HS-glycoprotein [Homo sapiens] | 3.79E−04 | 4502005 | 1 | 3 | 3 |
| 89 | alpha-2-glycoprotein 1, zinc; Alpha-2-glycoprotein, | 1.77E−08 | 4502337 | 2 | 3 | 3 |
| 90 | filensin; cytoskeletal protein, 115 KD [Homo sapie | 1.32E−09 | 4502399 | 3 | 3 | 1 |
| 91 | crystallin, beta A4; eye lens structural protein; C | 4.44E−15 | 4503059 | 8 | 3 | 2 |
| 92 | Fc fragment of IgG binding protein; IgG Fc binding | 1.36E−08 | 4503681 | 8 | 3 | 3 |
| 93 | glutathione transferase; deafness, X-linked 7; fatt | 1.03E−11 | 4504183 | 1 | 3 | 3 |
| 94 | alpha 2 globin; alpha globin; alpha-2 globin [Homo sapiens] | 6.97E−12 | 4504345 | 5 | 3 | 3 |
| 95 | lactotransferrin; lactoferrin [Homo sapiens] | 9.25E−11 | 4505043 | 11 | 3 | 3 |
| 96 | prolactin-induced protein; prolactin-inducible pro | 1.65E−09 | 4505821 | 2 | 3 | 3 |
| 97 | plasminogen [Homo sapiens] | 2.12E−04 | 4505881 | 1 | 3 | 2 |
| 98 | pregnancy-zone protein; Pregnancy zone protein [Homo sapiens] | 4.49E−09 | 4506355 | 1 | 3 | 3 |
| 99 | S-arrestin [Homo sapiens] | 9.37E−11 | 4506781 | 5 | 3 | 3 |
| 100 | acyl-Coenzyme A dehydrogenase, very long chain pre | 1.84E−04 | 4557235 | 2 | 3 | 3 |
| 101 | sparc/osteonectin, cwcv and kazal-like domains pro | 4.61E−06 | 4759164 | 1 | 3 | 3 |
| 102 | insulin receptor substrate 1 [Homo sapiens] | 3.11E−03 | 5031805 | 1 | 3 | 1 |
| 103 | cleavage and polyadenylation specific factor 3, 73 kDa; cleavage and pol | 1.10E−03 | 7706427 | 1 | 3 | 2 |
| 104 | leucine zipper, putative tumor suppressor 1; F37/E | 2.64E−03 | 10440566 | 1 | 3 | 2 |
| 105 | crystallin, gamma C; crystallin, gamma-3 [Homo sa | 1.39E−10 | 10518338 | 3 | 3 | 1 |
| 106 | succinate-CoA ligase, ADP-forming, beta subunit; SCS-betaA for ATP spe [symbol] | 5.79E−03 | 11321583 | 1 | 3 | 1 |
| 107 | alpha-2-plasmin inhibitor; alpha-2-antiplasmin [Homo sapiens] | 7.14E−12 | 11386143 | 1 | 3 | 3 |
| 108 | H4 histone family, member E [Homo sapiens] | 2.50E−06 | 11415030 | 1 | 3 | 3 |
| 109 | fibrinogen, gamma chain isoform gamma-B precursor [Homo sapiens] | 3.10E−11 | 11761633 | 6 | 3 | 2 |
| 110 | pantothenate kinase 3; pantothenic acid kinase [Homo sapiens] | 1.01E−03 | 13375789 | 1 | 3 | 2 |
| 111 | crystallin, gamma D; gamma crystallin 4 [Homo sap | 1.67E−14 | 13377002 | 3 | 3 | 1 |
| 112 | retbindin [Homo sapiens] | 1.13E−06 | 13899247 | 1 | 3 | 3 |
| 113 | ubiquitin specific protease 15; deubiquitinating enzyme [Homo sapiens] | 6.90E−04 | 14149627 | 1 | 3 | 2 |
| 114 | tubulin alpha 6 [Homo sapiens] | 4.32E−08 | 14389309 | 1 | 3 | 3 |
| 115 | leucine-rich alpha-2-glycoprotein 1; 2310031E04Rik | 8.88E−15 | 16418467 | 2 | 3 | 3 |
| 116 | peptidoglycan recognition protein L precursor [Homo sapiens] | 3.34E−05 | 21361845 | 1 | 3 | 3 |
| 117 | brain creatine kinase; creatine kinase B-chain; c | 1.13E−13 | 21536286 | 3 | 3 | 3 |
| 118 | junctophilin 1; mitsugumin72; junctophilin type1 [Homo sapiens] | 2.43E−03 | 21735575 | 1 | 3 | 2 |
| 119 | zinc finger protein 560 [Homo sapiens] | 1.43E−03 | 22749003 | 1 | 3 | 2 |
| 120 | DEAH (Asp-Glu-Ala-His) box polypeptide 29; nuclei | 2.79E−03 | 26553432 | 1 | 3 | 3 |
| 121 | nucleoporin 210; nuclear pore membrane glycoprotein 210 [Homo sapiens] | 2.84E−03 | 27477134 | 1 | 3 | 3 |
| 122 | PREDICTED: similar to Von Ebners gland protein pr | 1.42E−10 | 27498839 | 2 | 3 | 3 |
| 123 | hypothetical protein FLJ32440 [Homo sapiens] | 3.97E−05 | 27734761 | 1 | 3 | 2 |
| 124 | plexin B3; plexin-B3; plexin 6 [Homo sapiens] | 5.99E−04 | 29336063 | 1 | 3 | 1 |
| 125 | CP110 protein [Homo sapiens] | 6.19E−04 | 31543018 | 2 | 3 | 3 |
| 126 | pyruvate kinase 3 isoform 1; thyroid hormone-bind | 1.07E−10 | 33286418 | 2 | 3 | 3 |
| 127 | nucleoporin 214 kDa; nuclear pore complex protein | 1.49E−04 | 33946327 | 1 | 3 | 3 |
| 128 | bullous pemphigoid antigen 1 isoform 1; bullous p | 9.37E−03 | 34577047 | 1 | 3 | 3 |
| 129 | PREDICTED: similar to KIAA0663 gene product [Homo | 3.85E−04 | 41058123 | 1 | 3 | 3 |
| 130 | calsyntenin 1; non-classical cadherin XB31alpha1; | 1.69E−08 | 41281561 | 1 | 3 | 3 |
| 131 | amyloid beta A4 protein precursor, isoform b; pro | 6.60E−05 | 41406055 | 1 | 3 | 3 |
| 132 | PREDICTED: KIAA1030 protein [Homo sapiens] | 6.08E−04 | 42659643 | 1 | 3 | 3 |
| 133 | KIAA0317 [Homo sapiens] | 1.98E−03 | 42734315 | 1 | 3 | 2 |
| 134 | PREDICTED: similar to sin3 associated polypeptide p18 [Homo sapiens] | 4.51E−04 | 51461003 | 1 | 3 | 3 |
| 135 | PREDICTED: KIAA0367 protein [Homo sapiens] | 1.19E−03 | 51467193 | 1 | 3 | 3 |
| 136 | PREDICTED: similar to transcription elongation fa [symbol] | 2.45E−03 | 51467523 | 1 | 3 | 3 |
| 137 | complement component 9 [Homo sapiens] | 2.66E−05 | 4502511 | 1 | 2 | 2 |
| 138 | centromere protein C 1; Centromere autoantigen C1 [Homo sapiens] | 1.23E−03 | 4502779 | 1 | 2 | 2 |
| 139 | phakinin; beaded filament protein CP49; bfps2, Cyto | 9.69E−12 | 4502995 | 2 | 2 | 1 |
| 140 | cystatin S precursor; cystatin 4 [Homo sapiens] | 2.01E−06 | 4503109 | 1 | 2 | 2 |
| 141 | coagulation factor II precursor; prothrombin [Homo | 1.03E−09 | 4503635 | 1 | 2 | 2 |
| 142 | semaphorin 7A; sema domain, immunoglobulin domain | 1.10E−06 | 4504237 | 2 | 2 | 2 |
| 143 | insulin-like growth factor binding protein 7 [Homo | 3.61E−06 | 4504619 | 1 | 2 | 2 |
| 144 | neogenin homolog 1; neogenin (chicken) homolog 1 [Homo sapiens] | 3.72E−03 | 4505375 | 1 | 2 | 2 |
| 145 | prostatic binding protein; phosphatidylethanolamine | 1.66E−10 | 4505621 | 2 | 2 | 2 |
| 146 | phosphoglycerate kinase 1 [Homo sapiens] | 7.80E−06 | 4505763 | 2 | 2 | 2 |
| 147 | transketolase [Homo sapiens] | 3.22E−05 | 4507521 | 2 | 2 | 2 |
| 148 | biotinidase precursor [Homo sapiens] | 4.83E−07 | 4557373 | 1 | 2 | 2 |
| 149 | defensin, alpha 1 preproprotein; myeloid-related s [symbol] | 1.30E−04 | 4758146 | 1 | 2 | 2 |
| 150 | dual specificity phosphatase 8; serine/threonine specific protein phosp | 4.25E−05 | 4758212 | 1 | 2 | 2 |
| 151 | golgi autoantigen, golgin subfamily b, macrogolgin (with transmembrane | 5.05E−04 | 4758454 | 1 | 2 | 2 |
| 152 | growth factor receptor-bound protein 14 [Homo sapiens] | 3.32E−03 | 4758478 | 1 | 2 | 2 |
| 153 | nebulin [Homo sapiens] | 5.35E−03 | 4758794 | 1 | 2 | 2 |
| 154 | general transcription factor IIH, polypeptide 1 (62 kD subunit) [Homo sa | 5.41E−03 | 4885365 | 1 | 2 | 1 |
| 155 | macrophage erythroblast attacher; erythroblast macrophage protein [Homo | 4.84E−03 | 5031685 | 1 | 2 | 1 |
| 156 | galectin 3 binding protein; L3 antigen; Mac-2-bind | 2.94E−09 | 5031863 | 1 | 2 | 2 |
| 157 | elongation factor RNA polymerase II; ELL gene (11- | 6.28E−03 | 5729812 | 1 | 2 | 2 |
| 158 | chondromodulin I precursor; chondromodulin; BRICHOS | 4.77E−05 | 5901932 | 1 | 2 | 2 |
| 159 | coatomer protein complex, subunit gamma 2; coat pr | 7.04E−04 | 6912320 | 1 | 2 | 2 |
| 160 | WW domain binding protein 11; Npw38-binding protei | 4.17E−03 | 7706501 | 1 | 2 | 2 |
| 161 | H2A histone family, member A; histone H2AE [Homo | 4.17E−04 | 10645195 | 1 | 2 | 2 |
| 162 | retinoschisis (X-linked, juvenile) 1 [Homo sapiens | 6.93E−09 | 10835083 | 1 | 2 | 2 |
| 163 | metallothionein 1G [Homo sapiens] | 6.02E−06 | 10835230 | 1 | 2 | 2 |
| 164 | ubiquitin B precursor; polyubiquitin B [Homo sapie | 1.49E−05 | 11024714 | 2 | 2 | 2 |
| 165 | insulin-like growth factor binding protein 6 [Homo | 5.46E−04 | 11321593 | 1 | 2 | 2 |
| 166 | prosaposin (variant Gaucher disease and variant metachromatic leukodys [symbol] | 1.13E−04 | 11386147 | 1 | 2 | 2 |
| 167 | kallikrein 14 preproprotein; kallikrein-like protein 6 [Homo sapiens] | 3.25E−04 | 11545747 | 1 | 2 | 1 |
| 168 | fibrinogen, alpha chain isoform alpha preproprotei | 7.24E−08 | 11761629 | 5 | 2 | 2 |
| 169 | dynein, axonemal, intermediate polypeptide 2; dyn | 1.31E−03 | 12718866 | 1 | 2 | 2 |
| 170 | mitogen-activated protein kinase associated prote | 1.75E−03 | 13129138 | 1 | 2 | 2 |
| 171 | alpha 1 type II collagen isoform 1; collagen II, a | 4.47E−06 | 13435125 | 2 | 2 | 2 |
| 172 | serum amyloid A2 [Homo sapiens] | 2.77E−06 | 13540475 | 2 | 2 | 2 |
| 173 | hypothetical protein MGC4308 [Homo sapiens] | 7.51E−03 | 14150167 | 1 | 2 | 2 |
| 174 | lacritin precursor [Homo sapiens] | 6.51E−04 | 15187164 | 1 | 2 | 2 |
| 175 | ankyrin repeat domain 30A; breast cancer antigen NY-BR-1 [Homo sapiens | 3.16E−03 | 16506285 | 1 | 2 | 2 |
| 176 | ATPase, H+ transporting, lysosomal accessory prote | 8.95E−10 | 17136148 | 1 | 2 | 2 |
| 177 | keratin 4; Keratin-4; cytokeratin 4; keratin, typ | 4.40E−05 | 17318574 | 1 | 2 | 2 |
| 178 | tubulin, alpha 2 isoform 2 [Homo sapiens] | 2.17E−06 | 17921991 | 1 | 2 | 2 |
| 179 | vacuolar protein sorting 18 [Homo sapiens] | 3.62E−03 | 17978485 | 1 | 2 | 2 |
| 180 | vitronectin precursor; serum spreading factor; somatomedin B; compleme [symbol] | 4.77E−05 | 18201911 | 1 | 2 | 2 |
| 181 | SH3-domain binding protein 2; Cherubism [Homo sap | 1.87E−03 | 19923155 | 1 | 2 | 2 |
| 182 | proprotein convertase subtillsin/kexin type 2; sub | 1.13E−08 | 20336244 | 1 | 2 | 2 |
| 183 | chondroitin sulfate proteoglycan 2 (versican) [Hom | 1.19E−10 | 21361116 | 2 | 2 | 2 |
| 184 | aldehyde dehydrogenase 1A1; aldehyde dehydrogenase | 2.97E−07 | 21361176 | 3 | 2 | 1 |
| 185 | tubulin, beta, 5 [Homo sapiens] | 1.26E−12 | 21361322 | 1 | 2 | 2 |
| 186 | Tax1 (human T-cell leukemia virus type I) binding protein 1; tax1-bind | 1.14E−03 | 21361682 | 1 | 2 | 2 |
| 187 | RIC3 protein [Homo sapiens] | 5.90E−03 | 21362040 | 1 | 2 | 2 |
| 188 | hypothetical protein MGC27016 [Homo sapiens] | 1.48E−03 | 21450675 | 1 | 2 | 1 |
| 189 | zinc finger protein 625 [Homo sapiens] | 6.05E−03 | 21687161 | 1 | 2 | 1 |
| 190 | tyrosine 3/tryptophan 5-monooxygenase activation | 5.63E−10 | 21735625 | 1 | 2 | 2 |
| 191 | cathepsin B preproprotein; APP secretase; preproca | 2.76E−05 | 22538431 | 1 | 2 | 2 |
| 192 | spondin 1, extracellular matrix protein; spondin | 2.44E−04 | 24307905 | 1 | 2 | 2 |
| 193 | sperm associated antigen 6 isoform 2; sperm flagellar protein; axoneme | 2.37E−04 | 27262641 | 2 | 2 | 1 |
| 194 | SNF2 histone linker PHD RING helicase; 2610103K11 | 9.92E−04 | 27436873 | 1 | 2 | 2 |
| 195 | palmitoylated membrane protein 7 [Homo sapiens] | 5.12E−03 | 27735101 | 1 | 2 | 2 |
| 196 | eukaryotic translation initiation factor 2C, 3 isoform a; argonaute 3 | 3.35E−03 | 29294647 | 1 | 2 | 2 |
| 197 | hypothetical protein LOC92912 [Homo sapiens] | 2.50E−04 | 29789401 | 1 | 2 | 2 |
| 198 | PDZ domain containing 3 isoform a; PDZ domain con | 3.34E−05 | 30061507 | 1 | 2 | 2 |
| 199 | testis-specific protein TSP-NY isoform a [Homo sapiens] | 5.38E−03 | 30348972 | 1 | 2 | 2 |
| 200 | phosphatidylinositol-4-phosphate 5-kinase, type I, gamma; phosphatidyl | 3.65E−03 | 31317309 | 1 | 2 | 2 |
| 201 | polymeric immunoglobulin receptor; hepatocellular | 6.87E−12 | 31377806 | 4 | 2 | 2 |
| 202 | RNA-binding protein regulatory subunit; oncogene | 5.62E−10 | 31543380 | 1 | 2 | 1 |
| 203 | hypothetical protein FLJ38973 [Homo sapiens] | 1.93E−03 | 31581541 | 1 | 2 | 2 |
| 204 | Rho family guanine-nucleotide exchange factor [Ho | 4.32E−03 | 31742505 | 1 | 2 | 2 |
| 205 | cytosolic ovarian carcinoma antigen 1 isoform b; APK1 antigen [Homo sa [symbol] | 2.72E−03 | 32528291 | 1 | 2 | 2 |
| 206 | PH domain-containing protein [Homo sapiens] | 2.83E−04 | 33457316 | 1 | 2 | 2 |
| 207 | frizzled-related protein; Fritz; Frzb-1; fre; friz | 1.81E−03 | 38455388 | 1 | 2 | 2 |
| 208 | serum amyloid A1 isoform 2; tumor protein p53 indu [symbol] | 6.88E−14 | 40316910 | 2 | 2 | 2 |
| 209 | PREDICTED: zinc finger, CCHC domain containing 11 | 9.18E−05 | 41107445 | 1 | 2 | 1 |
| 210 | PREDICTED: similar to Phosphoglycerate mutase 1 ( | 7.24E−06 | 41149700 | 1 | 2 | 2 |
| 211 | semaphorin 3D; collapsin 2 [Homo sapiens] | 5.79E−04 | 41406086 | 1 | 2 | 2 |
| 212 | PREDICTED: hypothetical protein XP_375631 [Homo sa | 3.57E−03 | 42661664 | 1 | 2 | 2 |
| 213 | similar to RIKEN cDNA 1110018M03 [Homo sapiens] | 2.37E−03 | 42766422 | 1 | 2 | 2 |
| 214 | myoglobin [Homo sapiens] | 9.47E−07 | 44955885 | 1 | 2 | 2 |
| 215 | hypothetical protein FLJ31322 [Homo sapiens] | 1.29E−03 | 45387953 | 1 | 2 | 1 |
| 216 | FLJ42220 protein [Homo sapiens] | 4.37E−05 | 46409506 | 1 | 2 | 2 |
| 217 | zinc finger, MYND domain containing 11 isoform b; adenovirus 5 E1A bin | 2.30E−03 | 47078243 | 1 | 2 | 2 |
| 218 | hypothetical protein LOC255189 [Homo sapiens] | 2.73E−03 | 47106065 | 1 | 2 | 1 |
| 219 | PREDICTED: similar to ribosomal protein L31 [Homo sapiens] | 5.33E−04 | 51466239 | 1 | 2 | 2 |
| 220 | PREDICTED: similar to Olfactory receptor 52L1 [Homo sapiens] | 5.99E−03 | 51470915 | 1 | 2 | 1 |
| 221 | PREDICTED: similar to submaxillary apomucin [Homo | 5.14E−03 | 51471105 | 1 | 2 | 2 |
| 222 | PREDICTED: similar to hect domain and RLD 2 [Homo | 7.22E−04 | 51472707 | 1 | 2 | 2 |
| 223 | PREDICTED: hypothetical protein LOC146177 [Homo s | 9.52E−04 | 51472829 | 1 | 2 | 2 |
| 224 | PREDICTED: hypothetical protein XP_497598 [Homo s | 2.97E−03 | 51474534 | 1 | 2 | 2 |
| 225 | PREDICTED: hypothetical protein XP_498867 [Homo sapiens] | 1.37E−03 | 51475312 | 1 | 2 | 2 |
| 226 | PREDICTED: similar to Piccolo protein (Aczonin) [Homo sapiens] | 7.55E−04 | 51492882 | 1 | 2 | 2 |
| 227 | acetyl-Coenzyme A acyltransferase 1; peroxisomal 3 | 3.11E−03 | 4501853 | 1 | 1 | 1 |
| 228 | acetyl-Coenzyme A carboxylase beta [Homo sapiens] | 4.23E−03 | 4501855 | 1 | 1 | 1 |
| 229 | acyl-Coenzyme A oxidase 3, pristanoyl [Homo sapiens | 6.97E−03 | 4501871 | 1 | 1 | 1 |
| 230 | alkylglycerone phosphate synthase precursor [Homo sapiens] | 5.09E−03 | 4501993 | 1 | 1 | 1 |
| 231 | apolipoprotein B precursor; apoB-100; apoB-48 [Hom | 2.74E−09 | 4502153 | 2 | 1 | 1 |
| 232 | apolipoprotein D precursor [Homo sapiens] | 5.32E−03 | 4502163 | 1 | 1 | 1 |
| 233 | breast cancer 2, early onset; Fanconi anemia, comp | 6.95E−03 | 4502451 | 1 | 1 | 1 |
| 234 | complement component 1, r subcomponent [Homo sapie | 3.42E−09 | 4502493 | 1 | 1 | 1 |
| 235 | calbindin 2 full length protein isoform; calbindin | 9.84E−05 | 4502543 | 1 | 1 | 1 |
| 236 | calcium/calmodulin-dependent protein kinase IV; br | 4.02E−03 | 4502557 | 1 | 1 | 1 |
| 237 | corticosteroid binding globulin precursor; corticosteroid binding globu | 1.35E−03 | 4502595 | 1 | 1 | 1 |
| 238 | chromogranin B precursor; Chromogranin B (secretog | 7.55E−07 | 4502807 | 1 | 1 | 1 |
| 239 | cholinergic receptor, nicotinic, alpha polypeptide 2 (neuronal) [Homo s | 3.30E−03 | 4502823 | 1 | 1 | 1 |
| 240 | chloride channel 6 isoform CIC-6a [Homo sapiens] | 3.56E−03 | 4502873 | 1 | 1 | 1 |
| 241 | cellular repressor of E1A-stimulated genes [Homo sa | 3.41E−06 | 4503037 | 1 | 1 | 1 |
| 242 | cystatin B; stefin B; liver thiol proteinase inhib | 4.26E−09 | 4503117 | 1 | 1 | 1 |
| 243 | oral-facial-digital syndrome 1; chromosome X open | 7.51E−05 | 4503179 | 1 | 1 | 1 |
| 244 | dihydrofolate reductase [Homo sapiens] | 5.05E−03 | 4503323 | 1 | 1 | 1 |
| 245 | dihydropyrimidinase-like 2; collapsin response medi | 2.11E−03 | 4503377 | 1 | 1 | 1 |
| 246 | early growth response 1; nerve growth factor-induc | 9.73E−03 | 4503493 | 1 | 1 | 1 |
| 247 | enolase 1; MYC promoter-binding protein 1; non-neu | 2.01E−04 | 4503571 | 1 | 1 | 1 |
| 248 | coagulation factor XII precursor; Hageman factor [Homo sapiens] | 4.68E−03 | 4503629 | 1 | 1 | 1 |
| 249 | GDP dissociation inhibitor 1; mental retardation, | 2.24E−09 | 4503971 | 1 | 1 | 1 |
| 250 | glutaredoxin (thioltransferase) [Homo sapiens] | 3.18E−05 | 4504025 | 1 | 1 | 1 |
| 251 | gelsolin isoform a [Homo sapiens] | 7.98E−03 | 4504165 | 1 | 1 | 1 |
| 252 | general transcription factor IIE, polypeptide 2, b | 7.03E−06 | 4504195 | 1 | 1 | 1 |
| 253 | delta globin [Homo sapiens] | 4.67E−03 | 4504351 | 1 | 1 | 1 |
| 254 | complement factor H; H factor-1 (complement); fact | 2.72E−06 | 4504375 | 1 | 1 | 1 |
| 255 | interleukin 10 receptor, alpha precursor [Homo sapiens] | 4.94E−03 | 4504633 | 1 | 1 | 1 |
| 256 | interleukin 13 receptor, alpha 1 precursor; IL13 receptor alpha-1 chain | 6.69E−03 | 4504647 | 1 | 1 | 1 |
| 257 | inter-alpha (globulin) inhibitor H2; inter-alpha ( | 2.91E−03 | 4504783 | 1 | 1 | 1 |
| 258 | lactate dehydrogenase C [Homo sapiens] | 1.45E−07 | 4504973 | 1 | 1 | 1 |
| 259 | lumican [Homo sapiens] | 1.40E−04 | 4505047 | 1 | 1 | 1 |
| 260 | phospholipase C, gamma 2 (phosphatidylinositol-spe | 9.89E−03 | 4505871 | 1 | 1 | 1 |
| 261 | protein kinase C substrate 80K-H; glucosidase II, | 9.04E−03 | 4506077 | 1 | 1 | 1 |
| 262 | pleiotrophin; heparin affin regulatory protein; hep | 4.01E−05 | 4506281 | 1 | 1 | 1 |
| 263 | RAN binding protein 3 isoform RANBP3-a; RAN-bindin | 1.69E−03 | 4506409 | 1 | 1 | 1 |
| 264 | retinaldehyde binding protein 1; Newfoundland rod-c | 1.07E−08 | 4506541 | 1 | 1 | 1 |
| 265 | ribosomal protein S4, Y-linked 1 Y isoform; 40S ribosomal protein S4, Y | 8.87E−05 | 4506727 | 1 | 1 | 1 |
| 266 | ryanodine receptor 2 [Homo sapiens] | 9.67E−03 | 4506757 | 1 | 1 | 1 |
| 267 | secretory leukocyte protease inhibitor precursor; W | 4.40E−03 | 4507065 | 1 | 1 | 1 |
| 268 | synuclein, gamma (breast cancer-specific protein 1) | 1.25E−03 | 4507113 | 1 | 1 | 1 |
| 269 | superoxide dismutase 3, extracellular [Homo sapien | 3.79E−05 | 4507151 | 1 | 1 | 1 |
| 270 | SRY (sex determining region Y)-box 4; SRY-related HMG-box gene 4; ecotr | 5.28E−03 | 4507163 | 1 | 1 | 1 |
| 271 | thyrotrophic embryonic factor; thyrotroph embryonic factor [Homo sapien | 9.22E−03 | 4507431 | 1 | 1 | 1 |
| 272 | triosephosphate isomerase 1 [Homo sapiens] | 3.41E−07 | 4507645 | 1 | 1 | 1 |
| 273 | transformation/transcription domain-associated pro | 3.71E−03 | 4507691 | 1 | 1 | 1 |
| 274 | vimentin [Homo sapiens] | 6.52E−08 | 4507895 | 3 | 1 | 1 |
| 275 | apolipoprotein C-III precursor [Homo sapiens] | 1.13E−04 | 4557323 | 1 | 1 | 1 |
| 276 | B-cell lymphoma protein 2 beta isoform [Homo sapie | 2.71E−03 | 4557357 | 1 | 1 | 1 |
| 277 | cystathionine-beta-synthase; serine sulfhydrase; be | 6.11E−03 | 4557415 | 1 | 1 | 1 |
| 278 | CD14 antigen precursor [Homo sapiens] | 2.50E−04 | 4557417 | 1 | 1 | 1 |
| 279 | down-regulated in adenoma protein; down-regulated | 9.52E−03 | 4557535 | 1 | 1 | 1 |
| 280 | fatty acid binding protein 1, liver; Fatty acid-bin | 1.51E−05 | 4557577 | 1 | 1 | 1 |
| 281 | fatty acid binding protein 5 (psoriasis-associated) | 8.62E−03 | 4557581 | 1 | 1 | 1 |
| 282 | galactosylceramidase precursor; galactocerebrosidase; Galactosylceramin | 4.63E−04 | 4557613 | 1 | 1 | 1 |
| 283 | potassium voltage-gated channel, shaker-related subfamily, member 1 [Ho | 2.42E−03 | 4557685 | 1 | 1 | 1 |
| 284 | keratin 9 [Homo sapiens] | 1.89E−14 | 4557705 | 2 | 1 | 1 |
| 285 | myosin light chain 2 [Homo sapiens] | 5.53E−03 | 4557775 | 1 | 1 | 1 |
| 286 | propionyl-Coenzyme A carboxylase, alpha polypeptid | 8.42E−04 | 4557833 | 1 | 1 | 1 |
| 287 | general transcription factor IIIC, polypeptide 1, alpha 220 kDa; general | 9.27E−03 | 4753161 | 1 | 1 | 1 |
| 288 | chimerin (chimaerin) 2; Chimerin 2 (GTPase-activating protein, rho, 3); | 5.26E−03 | 4757980 | 1 | 1 | 1 |
| 289 | coatomer protein complex, subunit beta 2 (beta pri | 8.85E−03 | 4758032 | 1 | 1 | 1 |
| 290 | cellular retinoic acid binding protein 1; cellular | 1.25E−07 | 4758052 | 2 | 1 | 1 |
| 291 | doublecortin and CaM kinase-like 1; doublecortin-I | 9.42E−03 | 4758128 | 1 | 1 | 1 |
| 292 | desmoplakin; desmoplakin (DPI, DPII) [Homo sapiens | 1.81E−03 | 4758200 | 1 | 1 | 1 |
| 293 | cadherin 15 preproprotein; M-cadherin; muscle-cadherin; myotubule-cadhe | 4.15E−03 | 4826669 | 1 | 1 | 1 |
| 294 | fragile X mental retardation-related protein 1; Fragile X mental retard | 3.60E−03 | 4826736 | 1 | 1 | 1 |
| 295 | insulysin; insulinase [Homo sapiens] | 1.00E−02 | 4826770 | 1 | 1 | 1 |
| 296 | serine (or cysteine) proteinase inhibitor, clade I | 1.14E−03 | 4826904 | 1 | 1 | 1 |
| 297 | aldolase C, fructose-bisphosphate; Aldolase C, fruc | 1.31E−05 | 4885063 | 1 | 1 | 1 |
| 298 | crystallin, gamma B; crystallin, gamma 1-2 [Homo sa | 9.04E−06 | 4885157 | 1 | 1 | 1 |
| 299 | A-kinase anchor protein 8; A-kinase anchor protein, 95 kDa [Homo sapiens | 9.60E−03 | 5031579 | 1 | 1 | 1 |
| 300 | mitochondrial ribosomal protein S31; imogen 38 [Ho | 8.84E−03 | 5031787 | 1 | 1 | 1 |
| 301 | lactate dehydrogenase A [Homo sapiens] | 2.98E−03 | 5031857 | 1 | 1 | 1 |
| 302 | LIM domain kinase 2 isoform 2a [Homo sapiens] | 8.54E−03 | 5031869 | 1 | 1 | 1 |
| 303 | milk fat globule-EGF factor 8 protein; lactadherin | 4.40E−04 | 5174557 | 1 | 1 | 1 |
| 304 | polycystic kidney disease and receptor for egg jel | 9.42E−03 | 5174633 | 1 | 1 | 1 |
| 305 | metallothionein 3 [Homo sapiens] | 7.43E−04 | 5174762 | 1 | 1 | 1 |
| 306 | ADP-ribosylation factor guanine nucleotide-exchange factor 2; brefeldin | 8.25E−03 | 5453573 | 1 | 1 | 1 |
| 307 | structural maintenance of chromosomes 2-like 1; structural maintenance | 2.58E−03 | 5453591 | 1 | 1 | 1 |
| 308 | polymerase (DNA directed), delta 2, regulatory subunit; polymerase (DNA | 9.06E−03 | 5453924 | 1 | 1 | 1 |
| 309 | translocase of inner mitochondrial membrane 23 (yeast) homolog; translo | 5.15E−03 | 5454122 | 1 | 1 | 1 |
| 310 | beta-1,3-N-acetylglucosaminyltransferase bGnT-6; i | 1.65E−03 | 5802984 | 1 | 1 | 1 |
| 311 | enolase 2; neurone-specific enolase; neuron specifi | 3.25E−03 | 5803011 | 1 | 1 | 1 |
| 312 | transaldolase 1; glycerone transferase; dihydroxya | 5.62E−03 | 5803187 | 1 | 1 | 1 |
| 313 | follistatin-like 1 precursor; follistatin-related p | 3.56E−03 | 5901956 | 1 | 1 | 1 |
| 314 | polypeptide N-acetylgalactosaminyltransferase 6; Ga | 6.69E−03 | 6005766 | 1 | 1 | 1 |
| 315 | proline rich 4 (lacrimal); lacrimal proline rich pr | 2.37E−10 | 6005802 | 1 | 1 | 1 |
| 316 | RAN binding protein 2; nucleoporin 358; nuclear po | 1.51E−03 | 6382079 | 1 | 1 | 1 |
| 317 | golgi autoantigen, golgin subfamily a, 4; golgin-2 | 2.90E−03 | 6715600 | 1 | 1 | 1 |
| 318 | low density lipoprotein-related protein 2; megalin | 3.07E−04 | 6806919 | 1 | 1 | 1 |
| 319 | general transcription factor IIIC, polypeptide 3, | 6.08E−03 | 6912398 | 1 | 1 | 1 |
| 320 | rearranged L-myc fusion sequence; Zn-15 related [Homo sapiens] | 8.08E−03 | 6912632 | 1 | 1 | 1 |
| 321 | T-box 21; T-box expressed in T cells; T-cell-specific T-box transcripti [symbol] | 2.05E−03 | 7019549 | 1 | 1 | 1 |
| 322 | heparan sulfate proteoglycan 2; heparan sulfate pr | 3.21E−07 | 7427517 | 1 | 1 | 1 |
| 323 | golgi phosphoprotein 4; type II Golgi membrane prot | 3.82E−03 | 7657138 | 1 | 1 | 1 |
| 324 | MYB binding protein 1a; p53-activated protein-2 [Homo sapiens] | 6.25E−03 | 7657351 | 1 | 1 | 1 |
| 325 | sushi-repeat-containing protein, X-linked 2; sushi | 4.60E−04 | 7657619 | 1 | 1 | 1 |
| 326 | SH2 domain binding protein 1; TPR-containing, SH2- | 4.93E−03 | 7661950 | 1 | 1 | 1 |
| 327 | sparc/osteonectin, cwcv and kazal-like domains prot | 8.35E−04 | 7662036 | 1 | 1 | 1 |
| 328 | hepatoma-derived growth factor, related protein 3; hepatoma-derived gro | 3.24E−03 | 7705320 | 1 | 1 | 1 |
| 329 | F-box protein 40; muscle disease-related protein [H | 3.51E−04 | 7706493 | 1 | 1 | 1 |
| 330 | interphotoreceptor matrix proteoglycan 2; interpho | 2.29E−03 | 7706717 | 1 | 1 | 1 |
| 331 | TU12B1-TY protein [Homo sapiens] | 1.82E−03 | 7706749 | 1 | 1 | 1 |
| 332 | toll-like receptor 9 isoform A precursor [Homo sap | 5.19E−03 | 8394456 | 1 | 1 | 1 |
| 333 | hypothetical protein DKFZp434H2215 [Homo sapiens] | 1.96E−03 | 8922138 | 1 | 1 | 1 |
| 334 | mbt domain containing 1 [Homo sapiens] | 7.70E−03 | 8923059 | 1 | 1 | 1 |
| 335 | ankyrin repeat and SOCS box-containing 6 isoform 1 [Homo sapiens] | 2.02E−03 | 8923516 | 1 | 1 | 1 |
| 336 | hypothetical protein FLJ20574 [Homo sapiens] | 3.10E−04 | 8923539 | 1 | 1 | 1 |
| 337 | hypothetical protein FLJ20626 [Homo sapiens] | 3.88E−03 | 8923582 | 1 | 1 | 1 |
| 338 | ubinuclein 1 [Homo sapiens] | 4.83E−03 | 9055374 | 1 | 1 | 1 |
| 339 | DC12 protein [Homo sapiens] | 3.52E−03 | 9910182 | 1 | 1 | 1 |
| 340 | DC13 protein [Homo sapiens] | 3.77E−03 | 9910184 | 1 | 1 | 1 |
| 341 | S-adenosylhomocysteine hydrolase; adenosylhomocyst | 9.58E−03 | 9951915 | 1 | 1 | 1 |
| 342 | putatative 28 kDa protein [Homo sapiens] | 7.35E−04 | 10047140 | 1 | 1 | 1 |
| 343 | sodium channel, voltage-gated, type II, alpha 2; s | 2.80E−03 | 10337597 | 1 | 1 | 1 |
| 344 | nitric oxide synthase 1 (neuronal) [Homo sapiens] | 3.81E−03 | 10835173 | 1 | 1 | 1 |
| 345 | mitogen-activated protein kinase-activated protei | 3.28E−03 | 10863901 | 1 | 1 | 1 |
| 346 | tumor necrosis factor, alpha-induced protein 1 [Homo sapiens] | 5.50E−03 | 10863937 | 1 | 1 | 1 |
| 347 | tuberous sclerosis 2 isoform 2; tuberin isoform 1; tuberin isoform 2; | 2.96E−04 | 10938008 | 1 | 1 | 1 |
| 348 | ATP-binding cassette, sub-family F, member 1; ATP-binding cassette 50; | 6.21E−03 | 10947135 | 1 | 1 | 1 |
| 349 | meningioma expressed antigen 5 (hyaluronidase); hy | 8.71E−03 | 11024698 | 1 | 1 | 1 |
| 350 | guanine nucleotide-binding protein, beta-4 subuni | 4.03E−04 | 11055998 | 1 | 1 | 1 |
| 351 | bromodomain containing protein 3; open reading fr | 2.19E−03 | 11067749 | 1 | 1 | 1 |
| 352 | transforming, acidic coiled-coil containing prote | 9.91E−03 | 11119414 | 1 | 1 | 1 |
| 353 | protocadherin gamma subfamily B, 2 isoform 1 prec | 5.25E−04 | 11128035 | 1 | 1 | 1 |
| 354 | potassium channel, subfamily K, member 12; tandem pore domain potassiu [symbol] | 4.26E−04 | 11545761 | 1 | 1 | 1 |
| 355 | protease, serine, 22; protease, serine S1 family member 22; tryptase e [symbol] | 5.01E−03 | 11545839 | 1 | 1 | 1 |
| 356 | zinc finger protein 335; zinc-finger/leucine-zipp | 1.73E−03 | 11560152 | 1 | 1 | 1 |
| 357 | fibrinogen, beta chain preproprotein [Homo sapiens | 2.19E−06 | 11761631 | 2 | 1 | 1 |
| 358 | SH3 and multiple ankyrin repeat domains 1; somatostatin receptor-inter | 8.27E−04 | 11968152 | 1 | 1 | 1 |
| 359 | phosphoinositide-specific phospholipase C beta 1 | 4.07E−03 | 12083581 | 1 | 1 | 1 |
| 360 | hypothetical protein FLJ13868 [Homo sapiens] | 5.97E−03 | 12232403 | 1 | 1 | 1 |
| 361 | hypothetical protein FLJ12998 [Homo sapiens] | 1.16E−03 | 12232437 | 1 | 1 | 1 |
| 362 | solute carrier family 39 (zinc transporter), member 6; LIV-1 protein, | 3.83E−03 | 12751475 | 1 | 1 | 1 |
| 363 | hypothetical protein CG003 [Homo sapiens] | 7.59E−03 | 12957488 | 1 | 1 | 1 |
| 364 | DNA methyltransferase 1-associated protein 1 [Hom | 4.68E−03 | 13123776 | 1 | 1 | 1 |
| 365 | core-binding factor, beta subunit isoform 2; SL3/ | 6.19E−04 | 13124873 | 1 | 1 | 1 |
| 366 | betaine-homocysteine methyltransferase 2 [Homo sa | 9.24E−03 | 13162290 | 1 | 1 | 1 |
| 367 | hypothetical protein FLJ12875 [Homo sapiens] | 3.42E−03 | 13375705 | 1 | 1 | 1 |
| 368 | tubulin, alpha 4 [Homo sapiens] | 6.95E−05 | 13376539 | 1 | 1 | 1 |
| 369 | hypothetical protein FLJ21477 [Homo sapiens] | 4.46E−03 | 13376749 | 1 | 1 | 1 |
| 370 | serine (or cysteine) proteinase inhibitor, clade | 2.37E−03 | 13489087 | 1 | 1 | 1 |
| 371 | integrin-linked kinase-associated protein phosphat | 9.52E−03 | 13540531 | 1 | 1 | 1 |
| 372 | testis expressed sequence 13A [Homo sapiens] | 4.60E−03 | 13775180 | 1 | 1 | 1 |
| 373 | Rho GTPase activating protein 24 [Homo sapiens] | 2.35E−03 | 13775230 | 1 | 1 | 1 |
| 374 | hypothetical protein DKFZp434H0115 [Homo sapiens] | 7.76E−04 | 13899233 | 1 | 1 | 1 |
| 375 | mitochondrial ribosomal protein L32 [Homo sapiens | 5.55E−03 | 13994261 | 1 | 1 | 1 |
| 376 | spermiogenesis associated serine/threonine kinase | 8.60E−03 | 14042947 | 1 | 1 | 1 |
| 377 | coiled-coil domain containing 8 [Homo sapiens] | 5.64E−03 | 14042972 | 1 | 1 | 1 |
| 378 | heterogeneous nuclear ribonucleoprotein A1 isoform | 3.75E−07 | 14043070 | 1 | 1 | 1 |
| 379 | heterogeneous nuclear ribonucleoprotein A2/B1 iso | 6.82E−06 | 14043072 | 1 | 1 | 1 |
| 380 | heterogeneous nuclear ribonucleoprotein C isoform | 1.55E−04 | 14110428 | 1 | 1 | 1 |
| 381 | stromal cell-derived factor 2 precursor [Homo sapi | 1.79E−03 | 14141195 | 1 | 1 | 1 |
| 382 | latrophilin 3; latrophilin homolog 3 (cow); lectomedin 3 [Homo sapiens | 9.52E−03 | 14149677 | 1 | 1 | 1 |
| 383 | hypothetical protein DKFZp564J047 [Homo sapiens] | 8.44E−03 | 14149781 | 1 | 1 | 1 |
| 384 | solute carrier family 37 (glycerol-3-phosphate tra | 5.94E−04 | 14150047 | 1 | 1 | 1 |
| 385 | hypothetical protein MGC10854 [Homo sapiens] | 6.29E−03 | 14150056 | 1 | 1 | 1 |
| 386 | heterogeneous nuclear ribonucleoprotein K isoform | 2.08E−05 | 14165435 | 1 | 1 | 1 |
| 387 | hypothetical protein MGC10882 [Homo sapiens] | 6.22E−05 | 14249240 | 1 | 1 | 1 |
| 388 | internexin neuronal intermediate filament protein, | 3.10E−06 | 14249342 | 1 | 1 | 1 |
| 389 | hypothetical protein FLJ14768 [Homo sapiens] | 5.53E−03 | 14249548 | 1 | 1 | 1 |
| 390 | postreplication repair protein hRAD18p; RAD18, S. cerevisiae, homolog | 2.76E−03 | 14550405 | 1 | 1 | 1 |
| 391 | complement component 2 precursor; C3/C5 convertase [Homo sapiens] | 8.23E−04 | 14550407 | 1 | 1 | 1 |
| 392 | cadherin 2, type 1 preproprotein; cadherin 2, N-c | 5.73E−05 | 14589889 | 1 | 1 | 1 |
| 393 | centromere protein F (350/400 kD); centromere prot | 9.77E−03 | 14670381 | 1 | 1 | 1 |
| 394 | TATA box-binding protein-associated factor 2F; TA | 1.97E−04 | 14717407 | 1 | 1 | 1 |
| 395 | collagen, type VI, alpha 1 precursor; collagen VI | 1.85E−05 | 15011913 | 1 | 1 | 1 |
| 396 | brain expressed, X-linked 1; uncharacterized hypo | 6.54E−04 | 15147228 | 1 | 1 | 1 |
| 397 | keratin, hair, basic, 4; hard keratin, type II, 4 | 2.14E−03 | 15431316 | 1 | 1 | 1 |
| 398 | keratin 6 irs [Homo sapiens] | 1.43E−05 | 15618995 | 1 | 1 | 1 |
| 399 | vacuolar protein sorting 13A isoform B; chorein; | 3.86E−03 | 15619008 | 1 | 1 | 1 |
| 400 | E1A binding protein p400; CAGH32 protein; trinucleotide repeat contain | 4.47E−03 | 15805014 | 1 | 1 | 1 |
| 401 | cadherin 10, type 2 preproprotein; cadherin-10; T2-cadherin [Homo sapi | 9.94E−03 | 16306530 | 1 | 1 | 1 |
| 402 | cadherin 11, type 2 isoform 1 preproprotein; oste | 6.03E−03 | 16306532 | 1 | 1 | 1 |
| 403 | USH3A protein isoform c; clarin-1 [Homo sapiens] | 6.20E−03 | 16506281 | 1 | 1 | 1 |
| 404 | ubiquitin specific protease 28 [Homo sapiens] | 8.91E−03 | 16507200 | 1 | 1 | 1 |
| 405 | talin 1 [Homo sapiens] | 6.05E−03 | 16753233 | 1 | 1 | 1 |
| 406 | fibronectin 1 isoform 3 preproprotein; cold-insol | 2.17E−03 | 16933542 | 1 | 1 | 1 |
| 407 | VGF nerve growth factor inducible precursor; neuro | 8.47E−03 | 17136078 | 1 | 1 | 1 |
| 408 | keratin 1; Keratin-1; cytokeratin 1; hair alpha pr | 5.28E−05 | 17318569 | 2 | 1 | 1 |
| 409 | PREDICTED: KIAA0767 protein [Homo sapiens] | 8.86E−03 | 17485400 | 1 | 1 | 1 |
| 410 | vacuolar protein sorting 35; maternal-embryonic 3; vacuolar protein so | 4.10E−03 | 17999541 | 1 | 1 | 1 |
| 411 | protein tyrosine phosphatase, non-receptor type 3 | 7.93E−03 | 18104986 | 1 | 1 | 1 |
| 412 | GRB2-associated binding protein 2 isoform a; Grb2 | 3.47E−05 | 18105042 | 1 | 1 | 1 |
| 413 | PREDICTED: similar to synaptotagmin-like protein | 9.42E−03 | 18562480 | 1 | 1 | 1 |
| 414 | alpha 1 type XV collagen precursor; collagen XV, | 1.61E−03 | 18641350 | 1 | 1 | 1 |
| 415 | synaptosomal-associated protein 25 isoform SNAP25A | 8.87E−03 | 18765733 | 1 | 1 | 1 |
| 416 | protein tyrosine phosphatase, receptor type, K pr | 1.04E−03 | 18860902 | 1 | 1 | 1 |
| 417 | RAC/CDC42 exchange factor isoform 2 [Homo sapiens] | 4.60E−03 | 19311008 | 1 | 1 | 1 |
| 418 | actin related protein M1 [Homo sapiens] | 9.72E−03 | 19549325 | 1 | 1 | 1 |
| 419 | oxysterol-binding protein-like 1A isoform B; oxys | 8.02E−03 | 19718741 | 1 | 1 | 1 |
| 420 | solute carrier family 4, sodium bicarbonate cotra | 1.33E−03 | 19923176 | 1 | 1 | 1 |
| 421 | Jumonji, AT rich interactive domain 1B (RBP2-like | 3.81E−03 | 19923370 | 2 | 1 | 1 |
| 422 | adenylate kinase 3 alpha like; adenylate kinase 6 | 8.35E−03 | 19923437 | 1 | 1 | 1 |
| 423 | SLAM family member 7; CD2-like receptor activating cytotoxic cells; 19 | 5.67E−03 | 19923572 | 1 | 1 | 1 |
| 424 | hypothetical protein BC008207 [Homo sapiens] | 5.13E−03 | 19923911 | 1 | 1 | 1 |
| 425 | hypothetical protein BC011981 [Homo sapiens] | 6.90E−03 | 19923935 | 1 | 1 | 1 |
| 426 | TBP-associated factor 9L; neuronal cell death-rel | 6.83E−03 | 20070280 | 1 | 1 | 1 |
| 427 | numb homolog; numb (Drosophila) homolog [Homo sapi | 4.19E−03 | 20070356 | 1 | 1 | 1 |
| 428 | toll-like receptor 8 isoform 1 [Homo sapiens] | 2.90E−03 | 20302166 | 1 | 1 | 1 |
| 429 | ataxia telangiectasia mutated protein isoform 1; | 8.37E−03 | 20336203 | 1 | 1 | 1 |
| 430 | proprotein convertase subtilisin/kexin type 5 preproprotein; protease | 1.54E−04 | 20336246 | 1 | 1 | 1 |
| 431 | DEAH (Asp-Glu-Ala-His) box polypeptide 30 isoform | 3.17E−03 | 20336290 | 1 | 1 | 1 |
| 432 | PREDICTED: KIAA1604 protein [Homo sapiens] | 8.01E−04 | 20536216 | 1 | 1 | 1 |
| 433 | PREDICTED: PTPRF interacting protein, binding pro | 7.54E−03 | 20561108 | 1 | 1 | 1 |
| 434 | mitogen-activated protein kinase 10 isoform 3; MAP kinase; JNK3 alpha | 7.21E−03 | 20986506 | 1 | 1 | 1 |
| 435 | Kruppel-like factor 12 isoform a; KLF12 zinc fing | 4.42E−04 | 21071074 | 1 | 1 | 1 |
| 436 | testis nuclear RNA-binding protein [Homo sapiens] | 5.72E−03 | 21245124 | 1 | 1 | 1 |
| 437 | T-cell activation kelch repeat protein [Homo sapiens] | 5.41E−03 | 21245132 | 1 | 1 | 1 |
| 438 | ubiquitin carboxyl-terminal esterase L1 (ubiquiti | 1.47E−04 | 21361091 | 1 | 1 | 1 |
| 439 | enhancer of zeste 2 isoform a; enhancer of zeste | 8.14E−04 | 21361095 | 1 | 1 | 1 |
| 440 | four and a half LIM domains 1; Four-and-a-half LI | 2.85E−03 | 21361122 | 1 | 1 | 1 |
| 441 | CTP: phosphocholine cytidylyltransferase b [Homo s | 8.34E−03 | 21361202 | 1 | 1 | 1 |
| 442 | brain glycogen phosphorylase; glycogen phosphoryl | 1.32E−03 | 21361370 | 1 | 1 | 1 |
| 443 | BPY2 interacting protein 1; chromosome 19 open re | 3.61E−03 | 21361668 | 1 | 1 | 1 |
| 444 | transient receptor potential cation channel, subfamily M, member 8 [Ho | 3.58E−04 | 21361691 | 1 | 1 | 1 |
| 445 | PAP associated domain containing 1 [Homo sapiens] | 4.69E−04 | 21361704 | 1 | 1 | 1 |
| 446 | hypothetical protein FLJ10350 [Homo sapiens] | 1.61E−04 | 21361781 | 1 | 1 | 1 |
| 447 | DnaJ (Hsp40) homolog, subfamily C, member 1 [Homo sapiens] | 3.37E−03 | 21361912 | 1 | 1 | 1 |
| 448 | T-cell immunomodulatory protein [Homo sapiens] | 2.47E−05 | 21361933 | 1 | 1 | 1 |
| 449 | chromosome 10 open reading frame 87; Em: AC061711.1 [Homo sapiens] | 4.19E−03 | 21389373 | 1 | 1 | 1 |
| 450 | protease, serine, 15; Lon protease-like protein; | 9.76E−03 | 21396489 | 1 | 1 | 1 |
| 451 | immunoglobulin J chain [Homo sapiens] | 2.74E−06 | 21489959 | 1 | 1 | 1 |
| 452 | formyltetrahydrofolate dehydrogenase isoform a [H | 1.91E−03 | 21614513 | 1 | 1 | 1 |
| 453 | UDP-N-acetyl-alpha-D-galactosamine: polypeptide N-acetylgalactosaminylt [symbol] | 3.79E−03 | 21686971 | 1 | 1 | 1 |
| 454 | similar to hypothetical protein FLJ13659 [Homo sa | 9.12E−03 | 21687266 | 1 | 1 | 1 |
| 455 | lipoyltransferase 1 [Homo sapiens] | 4.13E−03 | 21729878 | 1 | 1 | 1 |
| 456 | sidekick 2; Drosophila sidekick-like; chicken sid | 8.27E−03 | 21735577 | 1 | 1 | 1 |
| 457 | PREDICTED: KIAA0882 protein [Homo sapiens] | 8.39E−04 | 22042713 | 1 | 1 | 1 |
| 458 | PREDICTED: follistatin-like 4 [Homo sapiens] | 5.31E−03 | 22049346 | 1 | 1 | 1 |
| 459 | NFAT activation molecule 1 precursor; calcinerin/ | 2.68E−03 | 22095335 | 1 | 1 | 1 |
| 460 | ankyrin repeat and SOCS box-containing protein 12 | 1.55E−04 | 22208955 | 2 | 1 | 1 |
| 461 | A-kinase anchor protein 9 isoform 2; A-kinase anc | 7.07E−03 | 22538387 | 1 | 1 | 1 |
| 462 | hypothetical protein DKFZp547B1713 [Homo sapiens] | 4.35E−03 | 22748815 | 1 | 1 | 1 |
| 463 | hypothetical protein FLJ35725 [Homo sapiens] | 2.51E−03 | 22749135 | 1 | 1 | 1 |
| 464 | zinc finger protein 553 [Homo sapiens] | 8.94E−03 | 22749325 | 1 | 1 | 1 |
| 465 | hypothetical protein FLJ25369 [Homo sapiens] | 1.38E−04 | 22749357 | 1 | 1 | 1 |
| 466 | aldehyde dehydrogenase 3 family, member A1; aldehy | 3.34E−03 | 22907049 | 1 | 1 | 1 |
| 467 | glutathione S-transferase M1 isoform 1; HB subuni | 2.98E−03 | 23065544 | 1 | 1 | 1 |
| 468 | elongation of very long chain fatty acids like 3 [Homo sapiens] | 1.06E−03 | 23097310 | 1 | 1 | 1 |
| 469 | hypothetical protein FLJ37440 [Homo sapiens] | 9.41E−03 | 23397471 | 1 | 1 | 1 |
| 470 | vitelliform macular dystrophy 2-like 2; bestrophin 4 [Homo sapiens] | 6.11E−03 | 23397576 | 1 | 1 | 1 |
| 471 | GREB1 protein isoform a; gene regulated by estrog | 3.50E−03 | 23397642 | 1 | 1 | 1 |
| 472 | mirror-image polydactyly 1; mirror-image polydact | 9.72E−03 | 23503325 | 1 | 1 | 1 |
| 473 | heat shock 70 kDa protein 8 isoform 2; heat shock c | 4.64E−03 | 24234686 | 1 | 1 | 1 |
| 474 | cytoplasmic FMR1 interacting protein 1; selective hybridizing clone (m [symbol] | 1.25E−03 | 24307969 | 1 | 1 | 1 |
| 475 | serine/threonine kinase 36 (fused homolog, Drosophila); serine/threoni∵ | 2.26E−03 | 24308123 | 1 | 1 | 1 |
| 476 | similar to hypothetical protein FLJ10883 [Homo sa | 2.09E−03 | 24308386 | 1 | 1 | 1 |
| 477 | MDN1, midasin homolog [Homo sapiens] | 5.39E−03 | 24415404 | 1 | 1 | 1 |
| 478 | steroid dehydrogenase-like [Homo sapiens] | 2.41E−03 | 24432037 | 1 | 1 | 1 |
| 479 | homeobox protein A5; homeobox protein HOXA5; home | 2.18E−03 | 24497517 | 1 | 1 | 1 |
| 480 | homeobox protein A10 isoform b; homeobox protein H | 2.19E−03 | 24497551 | 1 | 1 | 1 |
| 481 | aldo-keto reductase family 1, member C3; hydroxys | 1.28E−03 | 24497583 | 1 | 1 | 1 |
| 482 | myosin IIIA; deafness, autosomal recessive 30 [Homo sapiens] | 9.51E−04 | 24586657 | 1 | 1 | 1 |
| 483 | Y chromosome chromodomain protein 1 isoform a; te | 3.74E−03 | 25453481 | 1 | 1 | 1 |
| 484 | chromosome 19 open reading frame 21 [Homo sapiens | 9.72E−03 | 27735067 | 1 | 1 | 1 |
| 485 | membrane associated guanylate kinase interacting protein-like 1 [Homo | 9.00E−03 | 27735139 | 1 | 1 | 1 |
| 486 | chromosome 14 open reading frame 8; p25 beta [Homo sapiens] | 8.89E−03 | 27777659 | 1 | 1 | 1 |
| 487 | nuclear receptor subfamily 4, group A, member 3 is | 6.11E−03 | 27894355 | 1 | 1 | 1 |
| 488 | notch4 preproprotein; Notch (Drosophila) homolog 4 [Homo sapiens] | 5.09E−04 | 27894370 | 1 | 1 | 1 |
| 489 | cell adhesion molecule with homology to L1CAM pre | 2.66E−03 | 27894376 | 1 | 1 | 1 |
| 490 | zinc finger protein 545 [Homo sapiens] | 5.96E−03 | 28274686 | 1 | 1 | 1 |
| 491 | A-gamma globin; hemoglobin gamma-a chain; Hemoglo | 4.02E−05 | 28302131 | 2 | 1 | 1 |
| 492 | cell cycle progression 1; cell cycle progression | 7.64E−03 | 28395035 | 1 | 1 | 1 |
| 493 | Bardet-Biedl syndrome 1 [Homo sapiens] | 2.36E−03 | 28395045 | 1 | 1 | 1 |
| 494 | KIAA1404 protein [Homo sapiens] | 3.58E−03 | 28626521 | 1 | 1 | 1 |
| 495 | zinc finger, CW-type with coiled-coil domain 3; n | 9.24E−03 | 28872812 | 1 | 1 | 1 |
| 496 | TR4 orphan receptor associated protein TRA16; rep | 5.65E−03 | 28882043 | 1 | 1 | 1 |
| 497 | PREDICTED: similar to Tetratricopeptide repeat pr | 3.15E−03 | 29735853 | 1 | 1 | 1 |
| 498 | PREDICTED: huntingtin interacting protein-1-relat | 3.98E−03 | 29744338 | 1 | 1 | 1 |
| 499 | H63 breast cancer expressed gene isoform a; gene associated with HER-2 | 1.59E−03 | 29826289 | 1 | 1 | 1 |
| 500 | hypothetical protein FLJ40125 [Homo sapiens] | 5.33E−03 | 30425418 | 1 | 1 | 1 |
| 501 | neuroligin 2 [Homo sapiens] | 4.50E−03 | 30840978 | 1 | 1 | 1 |
| 502 | family with sequence similarity 29, member A [Homo sapiens] | 1.71E−04 | 31377562 | 1 | 1 | 1 |
| 503 | zinc finger protein 606 [Homo sapiens] | 9.52E−04 | 31377839 | 1 | 1 | 1 |
| 504 | heat shock protein apg-1 [Homo sapiens] | 4.66E−04 | 31541941 | 1 | 1 | 1 |
| 505 | inter-alpha (globulin) inhibitor H4 (plasma Kalli | 1.48E−08 | 31542984 | 2 | 1 | 1 |
| 506 | hypothetical protein MGC5601; similar to acyl-CoA dehydrogenase [Homo | 5.87E−04 | 31543201 | 1 | 1 | 1 |
| 507 | phosphoglycerate kinase 2; phosphoglycerate kinas | 1.23E−06 | 31543397 | 1 | 1 | 1 |
| 508 | cutaneous T-cell lymphoma tumor antigen se70-2 [H | 5.02E−03 | 31652264 | 1 | 1 | 1 |
| 509 | hypothetical protein LOC200186 [Homo sapiens] | 5.40E−03 | 32171215 | 1 | 1 | 1 |
| 510 | Ral GEF with PH domain and SH3 binding motif 2; Ral-A exchange factor | 3.25E−03 | 32441283 | 1 | 1 | 1 |
| 511 | peroxiredoxin 3 isoform b; antioxidant protein 1; thioredoxin-dependen [symbol] | 9.08E−03 | 32483377 | 1 | 1 | 1 |
| 512 | cytosolic phosphoenolpyruvate carboxykinase 1; phosphoenolpyruvate car [symbol] | 4.79E−03 | 32483401 | 1 | 1 | 1 |
| 513 | HECT domain containing 1 [Homo sapiens] | 2.40E−03 | 32698702 | 1 | 1 | 1 |
| 514 | cardiomyopathy associated 5; 2310076E16Rik [Homo sapiens] | 8.25E−03 | 32698780 | 1 | 1 | 1 |
| 515 | bromodomain adjacent to zinc finger domain, 1A isoform a; ATP-dependen [symbol] | 1.48E−03 | 32967603 | 1 | 1 | 1 |
| 516 | TUDOR gene similar [Homo sapiens] | 7.14E−03 | 32996737 | 1 | 1 | 1 |
| 517 | glycosyltransferase-like 1B; ortholog of mouse glycosyltransferase-lik | 9.33E−03 | 33285008 | 1 | 1 | 1 |
| 518 | biliverdin reductase A [Homo sapiens] | 1.86E−03 | 33589854 | 1 | 1 | 1 |
| 519 | phospholipase C gamma 1 isoform b; phospholipase | 3.00E−03 | 33598946 | 1 | 1 | 1 |
| 520 | LIM domain only 7; LOMP protein; zinc-finger doma | 1.21E−03 | 33598968 | 1 | 1 | 1 |
| 521 | a disintegrin and metalloproteinase with thrombos | 2.96E−03 | 33624885 | 1 | 1 | 1 |
| 522 | transmembrane channel-like 7 [Homo sapiens] | 5.84E−03 | 33636691 | 1 | 1 | 1 |
| 523 | hypothetical protein MGC35261 [Homo sapiens] | 1.07E−03 | 33859793 | 1 | 1 | 1 |
| 524 | kelch-like 8 [Homo sapiens] | 2.43E−03 | 34101268 | 1 | 1 | 1 |
| 525 | hypothetical protein MGC20781 [Homo sapiens] | 4.52E−03 | 34147472 | 1 | 1 | 1 |
| 526 | zinc finger protein 135 (clone pHZ-17); zinc finger protein 61 [Homo s | 8.75E−03 | 34419633 | 1 | 1 | 1 |
| 527 | alcohol dehydrogenase 1B (class I), beta polypept | 6.39E−06 | 34577061 | 1 | 1 | 1 |
| 528 | fibulin 1 isoform C precursor [Homo sapiens] | 4.72E−06 | 34734062 | 1 | 1 | 1 |
| 529 | ubiquitin specific protease 36 [Homo sapiens] | 6.71E−04 | 35250686 | 1 | 1 | 1 |
| 530 | nudix (nucleoside diphosphate linked moiety X)-type motif 11 [Homo sap | 4.64E−03 | 37221177 | 1 | 1 | 1 |
| 531 | sodium-dependent organic anion transporter [Homo sapiens] | 6.81E−05 | 37537552 | 1 | 1 | 1 |
| 532 | PREDICTED: intracellular membrane-associated calc | 1.19E−03 | 37538661 | 1 | 1 | 1 |
| 533 | PREDICTED: similar to pMesogenin1 [Homo sapiens] | 5.26E−03 | 37539465 | 1 | 1 | 1 |
| 534 | PREDICTED: similar to RIKEN cDNA 2210009G21 [Homo sapiens] | 4.97E−03 | 37556081 | 1 | 1 | 1 |
| 535 | KIAA0625 protein [Homo sapiens] | 5.44E−03 | 37620159 | 1 | 1 | 1 |
| 536 | AT-hook transcription factor; AT-hook transcripti | 6.81E−03 | 37620171 | 1 | 1 | 1 |
| 537 | SATB family member 2; two cut domains-containing | 3.24E−03 | 38016202 | 1 | 1 | 1 |
| 538 | similar to golgi autoantigen, golgin subfamily a, | 8.29E−03 | 38044110 | 1 | 1 | 1 |
| 539 | ring finger protein 31 [Homo sapiens] | 8.40E−03 | 35045940 | 1 | 1 | 1 |
| 540 | sister-of-mammalian grainyhead protein isoform 1; | 4.14E−03 | 38049007 | 1 | 1 | 1 |
| 541 | SET and MYND domain containing 1; CD8 beta opposi | 2.46E−03 | 38093643 | 1 | 1 | 1 |
| 542 | UNC13 (C. elegans)-like; homolog of rat Munc13 (d | 6.80E−03 | 38176154 | 1 | 1 | 1 |
| 543 | kelch-like 17; actinfilin [Homo sapiens] | 8.82E−03 | 38194229 | 1 | 1 | 1 |
| 544 | rotatin [Homo sapiens] | 1.91E−03 | 38201696 | 1 | 1 | 1 |
| 545 | hypothetical protein FLJ12178 [Homo sapiens] | 2.93E−03 | 38202219 | 1 | 1 | 1 |
| 546 | DEAD (Asp-Glu-Ala-Asp) box polypeptide 18; Myc-re | 6.32E−03 | 38327634 | 1 | 1 | 1 |
| 547 | FLJ46072 protein [Homo sapiens] | 7.24E−03 | 38348290 | 1 | 1 | 1 |
| 548 | FLJ46156 protein [Homo sapiens] | 1.33E−03 | 38348312 | 1 | 1 | 1 |
| 549 | inter-alpha (globulin) inhibitor HS-like; ITI-lik | 4.05E−03 | 38348336 | 1 | 1 | 1 |
| 550 | LOC161577 protein [Homo sapiens] | 4.15E−03 | 38348356 | 1 | 1 | 1 |
| 551 | lipocalin 2 (oncogene 24p3) [Homo sapiens] | 2.38E−11 | 38455402 | 3 | 1 | 1 |
| 552 | hypothetical protein LOC168850 [Homo sapiens] | 6.17E−03 | 39753953 | 1 | 1 | 1 |
| 553 | KIAA0052 [Homo sapiens] | 1.24E−05 | 39930353 | 1 | 1 | 1 |
| 554 | hypothetical protein BC013767 [Homo sapiens] | 8.80E−10 | 39930521 | 1 | 1 | 1 |
| 555 | KIAA1033 protein [Homo sapiens] | 6.37E−03 | 40018629 | 1 | 1 | 1 |
| 556 | formin-like 3 isoform 1; WW domain binding protei | 1.28E−03 | 40217839 | 1 | 1 | 1 |
| 557 | heat shock 90 kDa protein 1, alpha; heat shock 90k | 2.15E−05 | 40254816 | 1 | 1 | 1 |
| 558 | hypothetical protein FLJ10706 [Homo sapiens] | 7.51E−03 | 40254931 | 1 | 1 | 1 |
| 559 | zinc finger protein 295 [Homo sapiens] | 8.35E−03 | 40254945 | 1 | 1 | 1 |
| 560 | hypothetical protein FLJ21019 [Homo sapiens] | 7.94E−03 | 40255047 | 1 | 1 | 1 |
| 561 | hypothetical protein MGC34732 [Homo sapiens] | 9.92E−05 | 40255180 | 1 | 1 | 1 |
| 562 | retinitis pigmentosa 1-like 1 [Homo sapiens] | 5.54E−03 | 40255278 | 1 | 1 | 1 |
| 563 | thrombospondin 2 precursor [Homo sapiens] | 6.85E−03 | 40317628 | 1 | 1 | 1 |
| 564 | PREDICTED: similar to Triosephosphate isomerase (T | 1.24E−05 | 41058276 | 1 | 1 | 1 |
| 565 | PREDICTED: TBP-interacting protein [Homo sapiens] | 6.46E−03 | 41144277 | 1 | 1 | 1 |
| 566 | PREDICTED: hypothetical protein LOC285335 [Homo sapiens] | 2.58E−03 | 41146536 | 1 | 1 | 1 |
| 567 | PREDICTED: hypothetical protein XP_370638 [Homo s | 3.28E−03 | 41149556 | 1 | 1 | 1 |
| 568 | PREDICTED: similar to serologically defined colon | 4.05E−03 | 41150943 | 1 | 1 | 1 |
| 569 | PREDICTED: agrin [Homo sapiens] | 6.20E−04 | 41151826 | 1 | 1 | 1 |
| 570 | U5 snRNP-specific protein, 116 kD [Homo sapiens] | 1.88E−03 | 41152056 | 1 | 1 | 1 |
| 571 | PREDICTED: KIAA1549 protein [Homo sapiens] | 8.65E−03 | 41199251 | 1 | 1 | 1 |
| 572 | neuronal cell adhesion molecule; Bravo [Homo sapie | 5.84E−07 | 41281389 | 1 | 1 | 1 |
| 573 | importin 13; Ran binding protein 13; karyopherin 13 [Homo sapiens] | 1.49E−04 | 41281425 | 1 | 1 | 1 |
| 574 | KIAA0433 protein [Homo sapiens] | 2.12E−03 | 41281583 | 1 | 1 | 1 |
| 575 | crumbs homolog 1 isoform II precursor; crumbs (Dr | 2.45E−03 | 41327708 | 1 | 1 | 1 |
| 576 | ATPase, Class II, type 9B; ATPase type IV, phosph | 3.40E−03 | 41327760 | 1 | 1 | 1 |
| 577 | Rho-specific guanine nucleotide exchange factor p | 6.68E−03 | 41327769 | 1 | 1 | 1 |
| 578 | proline arginine-rich end leucine-rich repeat prot | 7.89E−03 | 41349454 | 1 | 1 | 1 |
| 579 | PR domain containing 5 [Homo sapiens] | 9.30E−03 | 41349476 | 1 | 1 | 1 |
| 580 | calsyntenin 3; alcadein [Homo sapiens] | 2.23E−03 | 42475534 | 1 | 1 | 1 |
| 581 | nuclear receptor coactivator 7; estrogen receptor associated protein 1 | 6.25E−03 | 42476175 | 1 | 1 | 1 |
| 582 | vacuolar protein sorting 13C protein [Homo sapiens] | 3.48E−03 | 42544121 | 1 | 1 | 1 |
| 583 | galectin 8 isoform a; Po66 carbohydrate binding p | 2.45E−03 | 42544185 | 1 | 1 | 1 |
| 584 | complement factor D preproprotein; adipsin; prope | 1.32E−04 | 42544239 | 1 | 1 | 1 |
| 585 | PREDICTED: myosin VI [Homo sapiens] | 7.97E−03 | 42657517 | 1 | 1 | 1 |
| 586 | PREDICTED: similar to tudor domain containing 6 protein [Homo sapiens] | 1.40E−03 | 42657656 | 1 | 1 | 1 |
| 587 | PREDICTED: hypothetical protein FLJ11811 [Homo sa | 9.02E−03 | 42660246 | 1 | 1 | 1 |
| 588 | PREDICTED: KIAA0753 gene product [Homo sapiens] | 1.96E−03 | 42661078 | 1 | 1 | 1 |
| 589 | PREDICTED: ProSAPiP2 protein [Homo sapiens] | 3.74E−03 | 42661180 | 1 | 1 | 1 |
| 590 | PREDICTED: similar to KIAA1074 protein [Homo sapiens] | 9.67E−03 | 42661355 | 1 | 1 | 1 |
| 591 | MAS-related GPR, member D; mas-related G protein-coupled MRGD [Homo | 2.48E−03 | 42794265 | 1 | 1 | 1 |
| | sa [symbol] | | | | | |
| 592 | zinc finger protein 262; cell death inhibiting RN | 4.80E−04 | 44890068 | 1 | 1 | 1 |
| 593 | mitofusin 1 isoform 1; putative transmembrane GTPase [Homo sapiens] | 9.39E−03 | 45269137 | 1 | 1 | 1 |
| 594 | limbic system-associated membrane. protein [Homo sa | 3.01E−05 | 45594240 | 1 | 1 | 1 |
| 595 | ring finger and WD repeat domain 1 isoform 1; rin | 9.78E−03 | 45594312 | 1 | 1 | 1 |
| 596 | hypothetical protein FLJ21918 [Homo sapiens] | 2.54E−03 | 45935393 | 1 | 1 | 1 |
| 597 | bifunctional phosphopantetheine adenylyl transferase/dephospho CoA kin | 7.22E−03 | 46048207 | 1 | 1 | 1 |
| 598 | M-phase phosphoprotein 1; mitotic kinesin-like protein [Homo sapiens] | 4.11E−03 | 46049114 | 1 | 1 | 1 |
| 599 | KIAA1632 protein [Homo sapiens] | 7.53E−03 | 46195733 | 1 | 1 | 1 |
| 600 | unc-13 homolog D [Homo sapiens] | 6.22E−03 | 46195765 | 1 | 1 | 1 |
| 601 | latent transforming growth factor beta binding pr | 3.69E−03 | 46249412 | 1 | 1 | 1 |
| 602 | FLJ45949 protein [Homo sapiens] | 9.25E−03 | 46409412 | 1 | 1 | 1 |
| 603 | FLJ45248 protein [Homo sapiens] | 7.98E−03 | 46409638 | 1 | 1 | 1 |
| 604 | succinate receptor 1; G protein-coupled receptor | 5.22E−03 | 47271392 | 1 | 1 | 1 |
| 605 | leucine-zipper-like transcriptional regulator, 1; | 5.48E−03 | 47717139 | 1 | 1 | 1 |
| 606 | PREDICTED: similar to RIKEN cDNA 1700013E18 [Homo | 2.07E−03 | 51458576 | 1 | 1 | 1 |
| 607 | PREDICTED: similar to Fc receptor homolog expressed in B cells; Fc rec | 7.24E−03 | 51458812 | 1 | 1 | 1 |
| 608 | PREDICTED: hypothetical cardiac/skeletal muscle-e | 1.41E−03 | 51458828 | 1 | 1 | 1 |
| 609 | PREDICTED: similar to Putative dimethylaniline mo | 1.85E−04 | 51458831 | 1 | 1 | 1 |
| 610 | PREDICTED: similar to osteotesticular protein tyr | 7.58E−03 | 51458868 | 1 | 1 | 1 |
| 611 | PREDICTED: similar to elongation factor 1 delta [ | 2.64E−03 | 51459015 | 1 | 1 | 1 |
| 612 | PREDICTED: similar to 40S ribosomal protein S7 (S8) [Homo sapiens] | 2.47E−03 | 51459063 | 1 | 1 | 1 |
| 613 | PREDICTED: hypothetical protein XP_498801 [Homo sa | 5.30E−03 | 51459364 | 1 | 1 | 1 |
| 614 | PREDICTED: similar to bA395L14.5 (novel phosphogl | 5.85E−03 | 51460421 | 1 | 1 | 1 |
| 615 | PREDICTED: hypothetical protein XP_295058 [Homo s | 3.34E−03 | 51460438 | 1 | 1 | 1 |
| 616 | PREDICTED: similar to Calpain 9 (Digestive tract- | 4.22E−03 | 51460556 | 1 | 1 | 1 |
| 617 | PREDICTED: similar to anaphase promoting complex | 1.36E−03 | 51460657 | 1 | 1 | 1 |
| 618 | PREDICTED: similar to Ig kappa variable region [Ho | 1.87E−03 | 51460659 | 1 | 1 | 1 |
| 619 | PREDICTED: similar to KIAA0445 protein [Homo sapi | 4.00E−03 | 51461034 | 1 | 1 | 1 |
| 620 | PREDICTED: twist homolog 2 [Homo sapiens] | 4.71E−03 | 51463853 | 1 | 1 | 1 |
| 621 | PREDICTED: hypothetical protein XP_498937 [Homo sapiens] | 3.47E−03 | 51463864 | 1 | 1 | 1 |
| 622 | PREDICTED: FYVE, RhoGEF and PH domain containing 5 [Homo sapiens] | 9.64E−03 | 51463913 | 1 | 1 | 1 |
| 623 | PREDICTED: KIAA0226 gene product [Homo sapiens] | 9.79E−03 | 51464047 | 1 | 1 | 1 |
| 624 | PREDICTED: similar to KIAA2033 protein [Homo sapi | 7.94E−03 | 51464267 | 1 | 1 | 1 |
| 625 | PREDICTED: hypothetical protein DKFZp762K222 [Homo sapiens] | 7.47E−03 | 51464327 | 1 | 1 | 1 |
| 626 | PREDICTED: similar to apopolysialoglycoprotein- | 8.72E−03 | 51464378 | 1 | 1 | 1 |
| 627 | PREDICTED: similar to RIKEN cDNA G431001E03 gene [Homo sapiens] | 4.73E−03 | 51464570 | 1 | 1 | 1 |
| 628 | PREDICTED: similar to FLJ14624 protein [Homo sapi | 2.65E−03 | 51464636 | 1 | 1 | 1 |
| 629 | PREDICTED: similar to bA110H4.2 (similar to membr | 3.97E−03 | 51464932 | 1 | 1 | 1 |
| 630 | PREDICTED: similar to membrane-spanning proteogly | 5.64E−03 | 51464985 | 1 | 1 | 1 |
| 631 | PREDICTED: T-box 18 [Homo sapiens] | 5.28E−03 | 51465420 | 1 | 1 | 1 |
| 632 | PREDICTED: hypothetical protein XP_499090 [Homo s | 9.07E−03 | 51465873 | 1 | 1 | 1 |
| 633 | PREDICTED: similar to KIAA0877 protein [Homo sapie | 1.15E−03 | 51466121 | 1 | 1 | 1 |
| 634 | PREDICTED: KIAA0543 protein [Homo sapiens] | 1.13E−03 | 51466599 | 1 | 1 | 1 |
| 635 | PREDICTED: hypothetical protein XP_499235 [Homo s | 2.79E−03 | 51466834 | 1 | 1 | 1 |
| 636 | PREDICTED: similar to Heat shock cognate 71 kDa pr | 1.20E−03 | 51466978 | 1 | 1 | 1 |
| 637 | PREDICTED: potassium channel, subfamily T, member | 3.46E−03 | 51467170 | 1 | 1 | 1 |
| 638 | PREDICTED: KIAA0368 protein [Homo sapiens] | 3.74E−04 | 51467589 | 1 | 1 | 1 |
| 639 | PREDICTED: similar to bA526D8.2 (novel protein si | 6.51E−03 | 51467642 | 1 | 1 | 1 |
| 640 | PREDICTED: neuregulin 3 [Homo sapiens] | 6.31E−03 | 51467770 | 1 | 1 | 1 |
| 641 | PREDICTED: similar to tigger transposable element | 5.47E−03 | 51467790 | 1 | 1 | 1 |
| 642 | PREDICTED: similar to centaurin, gamma-like family, 1 member 1; ARF GTPa | 6.34E−03 | 51468155 | 1 | 1 | 1 |
| 643 | PREDICTED: GCN1 general control of amino-acid synthesis 1-like 1 [Homo | 2.23E−03 | 51471344 | 1 | 1 | 1 |
| 644 | PREDICTED: similar to Golgi autoantigen, golgin subfamily A member 6 ( | 5.11E−03 | 51472431 | 1 | 1 | 1 |
| 645 | PREDICTED: similar to Ig heavy chain V-I region HG3 precursor [Homo sa | 7.98E−03 | 51472657 | 1 | 1 | 1 |
| 646 | PREDICTED: similar to elongation factor Tu GTP bin | 7.13E−03 | 51472670 | 1 | 1 | 1 |
| 647 | PREDICTED: similar to ENSANGP0000013733 [Homo sa | 3.85E−03 | 51473057 | 1 | 1 | 1 |
| 648 | PREDICTED: hypothetical protein XP_370924 [Homo s | 1.74E−03 | 51473154 | 1 | 1 | 1 |
| 649 | PREDICTED: FLJ46675 protein [Homo sapiens] | 6.48E−03 | 51474152 | 1 | 1 | 1 |
| 650 | PREDICTED: similar to 60S ribosomal protein L23a [Homo sapiens] | 6.71E−04 | 51474226 | 1 | 1 | 1 |
| 651 | PREDICTED: 5-azacytidine induced 1 [Homo sapiens] | 9.17E−03 | 51474428 | 1 | 1 | 1 |
| 652 | PREDICTED: similar to contains transmembrane (TM) | 4.67E−03 | 51474648 | 1 | 1 | 1 |
| 653 | PREDICTED: KIAA1205 [Homo sapiens] | 6.79E−03 | 51474761 | 1 | 1 | 1 |
| 654 | PREDICTED: hypothetical protein LOC284371 [Homo s | 1.79E−03 | 51474776 | 1 | 1 | 1 |
| 655 | PREDICTED: SHC (Src homology 2 domain containing) | 6.77E−03 | 51474945 | 1 | 1 | 1 |
| 656 | PREDICTED: similar to eukaryotic translation elo | 7.50E−03 | 51474994 | 1 | 1 | 1 |
| 657 | PREDICTED: similar to Ig kappa chain [Homo sapien | 1.91E−03 | 51475407 | 1 | 1 | 1 |
| 658 | PREDICTED: KIAA0542 gene product [Homo sapiens] | 9.15E−03 | 51476003 | 1 | 1 | 1 |
| 659 | PREDICTED: similar to RIKEN cDNA 1700019P01 [Homo | 1.39E−04 | 51476104 | 1 | 1 | 1 |
| 660 | PREDICTED: similar to bA203I16.1 (KIAA0970 protei | 3.10E−03 | 51477225 | 1 | 1 | 1 |
| 661 | PREDICTED: hypothetical protein XP_098980 [Homo s | 1.51E−03 | 51477357 | 1 | 1 | 1 |
| 662 | PREDICTED: similar to sperm protein associated wi | 3.79E−03 | 51477491 | 1 | 1 | 1 |
| 663 | PREDICTED: hypothetical protein XP_499220 [Homo sa | 4.15E−03 | 51492740 | 1 | 1 | 1 |
| 664 | PREDICTED: YLP motif containing 1 [Homo sapiens] | 2.00E−03 | 51493127 | 1 | 1 | 1 |

TABLE 3.2
Proteins identified in vitreous fluids (II)

| | | P | Accession | Id | Id No | Pat |
|---|---|---|---|---|---|---|
| | Protein Name | (probability) | No | Pep | on 19 | No |
| 1 | albumin precursor; PRO0883 protein [Homo sapiens] | 1.00E−30 | 4502027 | 95 | 19 | 11 |
| 2 | transthyretin; prealbumin [Homo sapiens] | 1.00E−30 | 4507725 | 23 | 19 | 11 |
| 3 | gelsolin isoform b [Homo sapiens] | 1.00E−30 | 38044288 | 4 | 14 | 9 |
| 4 | tripeptidyl-peptidase I precursor [Homo sapiens] | 1.00E−30 | 5729770 | 1 | 7 | 7 |
| 5 | serine (or cysteine) proteinase inhibitor, clade A (alpha-1 antiprotei | 1.11E−16 | 21361198 | 16 | 19 | 11 |
| 6 | transferrin; PRO2086 protein [Homo sapiens] | 1.11E−15 | 4557871 | 52 | 19 | 11 |
| 7 | crystallin, beta A3; eye lens structural protein [Homo sapiens] | 2.22E−15 | 12056461 | 11 | 7 | 2 |
| 8 | RBP3 gene product [Homo sapiens] | 3.33E−15 | 4506453 | 16 | 14 | 10 |
| 9 | crystallin, beta A4; eye lens structural protein; C | 4.44E−15 | 4503059 | 8 | 3 | 2 |
| 10 | crystallin, alpha A; crystallin, alpha-1; human alphaA-crystallin (CRYA | 6.66E−15 | 4503055 | 9 | 6 | 2 |
| 11 | dickkopf homolog 3; RIG-like 7-1; RIG-like 5-6; dickkopf (Xenopus laev | 7.77E−15 | 40548389 | 3 | 10 | 9 |
| 12 | enolase 3; enolase-3, beta, muscle; muscle specif | 8.88E−15 | 16554592 | 1 | 4 | 4 |
| 13 | leucine-rich alpha-2-glycoprotein 1; 2310031E04Rik | 8.88E−15 | 16418467 | 2 | 3 | 3 |
| 14 | beta-2-microglobulin precursor [Homo sapiens] | 9.99E−15 | 4757826 | 3 | 14 | 10 |
| 15 | lysozyme precursor [Homo sapiens] | 9.99E−15 | 4557894 | 5 | 6 | 6 |
| 16 | beta globin; hemoglobin beta chain [Homo sapiens] | 9.99E−15 | 4504349 | 6 | 5 | 4 |
| 17 | crystallin, gamma D; gamma crystallin 4 [Homo sap | 1.67E−14 | 13377002 | 3 | 3 | 1 |
| 18 | keratin 9 [Homo sapiens] | 1.89E−14 | 4557705 | 2 | 1 | 1 |
| 19 | ectonucleotide pyrophosphatase/phosphodiesterase 2 | 2.66E−14 | 20070230 | 2 | 5 | 5 |
| 20 | complement component 4B proprotein [Homo sapiens] | 3.11E−14 | 4502501 | 6 | 16 | 11 |
| 21 | secreted phosphoprotein 1 (osteopontin, bone slalo | 3.44E−14 | 4759166 | 5 | 9 | 9 |
| 22 | beta-2-glycoprotein I precursor [Homo sapiens] | 5.22E−14 | 4557327 | 4 | 10 | 5 |
| 23 | prostaglandin D2 synthase 21 kDa; prostaglandin D synthase; prostagland [symbol] | 5.88E−14 | 32171249 | 3 | 10 | 8 |
| 24 | hemopexin [Homo sapiens] | 6.22E−14 | 11321561 | 9 | 18 | 10 |
| 25 | serum amyloid A1 isoform 2; tumor protein p53 indu | 6.88E−14 | 40316910 | 2 | 2 | 2 |
| 26 | complement component 3 precursor; acylation-stimulating protein cleavag [symbol] | 8.55E−14 | 4557385 | 10 | 18 | 10 |
| 27 | RBP4 gene product [Homo sapiens] | 9.77E−14 | 5803139 | 3 | 8 | 6 |
| 28 | brain creatine kinase; creatine kinase B-chain; c | 1.13E−13 | 21536286 | 3 | 3 | 3 |
| 29 | vitamin D-binding protein precursor; vitamin D-binding alpha-globulin | 1.49E−13 | 32483410 | 7 | 17 | 9 |
| 30 | clusterin isoform 1; complement-associated protein SP-40 [Homo sapiens | 1.53E−13 | 42716297 | 14 | 19 | 11 |
| 31 | orosomucoid 2; alpha-1-acid glycoprotein, type 2 [Homo sapiens] | 2.73E−13 | 4505529 | 5 | 17 | 10 |
| 32 | orosomucoid 1 precursor; alpha-1-acid glycoprotein 1; Orosomucoid-1 (al [symbol] | 5.08E−13 | 9257232 | 6 | 15 | 10 |
| 33 | crystallin, gamma S; crystallin, gamma 8 [Homo sapiens] | 8.39E−13 | 8922120 | 12 | 6 | 3 |
| 34 | serine (or cysteine) proteinase inhibitor, clade F (alpha-2 antiplasmi | 1.05E−12 | 39725934 | 13 | 18 | 11 |
| 35 | afamin precursor; alpha-albumin [Homo sapiens] | 1.10E−12 | 4501987 | 1 | 4 | 3 |
| 36 | lipocalin 1 precursor; lipocalin 1 (protein migrati | 1.23E−12 | 4504963 | 4 | 6 | 6 |
| 37 | tubulin, beta, 5 [Homo sapiens] | 1.26E−12 | 21361322 | 1 | 2 | 2 |
| 38 | alpha 1B-glycoprotein [Homo sapiens] | 2.60E−12 | 21071030 | 2 | 8 | 6 |
| 39 | angiotensinogen precursor; angiotensin II precursor; pre-angiotensinoge | 2.70E−12 | 4557287 | 4 | 6 | 6 |
| 40 | ceruloplasmin (ferroxidase); Ceruloplasmin [Homo sapiens] | 2.96E−12 | 4557485 | 8 | 18 | 11 |
| 41 | apolipoprotein A-I precursor [Homo sapiens] | 4.91E−12 | 4557321 | 10 | 16 | 10 |
| 42 | alpha-2-macroglobulin precursor [Homo sapiens] | 5.16E−12 | 4557225 | 8 | 10 | 10 |
| 43 | polymeric Immunoglobulin receptor; hepatocellular | 6.87E−12 | 31377806 | 4 | 2 | 2 |
| 44 | alpha 2 globin; alpha globin; alpha-2 globin [Homo sapiens] | 6.97E−12 | 4504345 | 5 | 3 | 3 |
| 45 | carboxypeptidase E precursor [Homo sapiens] | 7.12E−12 | 4503009 | 4 | 5 | 5 |
| 46 | alpha-2-plasmin inhibitor; alpha-2-antiplasmin [Homo sapiens] | 7.14E−12 | 11386143 | 1 | 3 | 3 |
| 47 | phakinin; beaded filament protein CP49; bfps2, Cyto | 9.69E−12 | 4502995 | 2 | 2 | 1 |
| 48 | EGF-containing fibulin-like extracellular matrix protein 1 isoform b; f | 1.03E−11 | 9665253 | 3 | 13 | 9 |
| 49 | glutathione transferase; deafness, X-linked 7; fatt | 1.03E−11 | 4504183 | 1 | 3 | 3 |
| 50 | plasma glutathione peroxidase 3 precursor [Homo sapiens] | 1.54E−11 | 6006001 | 2 | 9 | 7 |
| 51 | crystallin, beta B1; eye lens structural protein [Homo sapiens] | 1.56E−11 | 4503061 | 22 | 8 | 3 |
| 52 | apolipoprotein E precursor; apolipoprotein E3 [Homo sapiens] | 1.58E−11 | 4557325 | 6 | 13 | 9 |
| 53 | apolipoprotein A-II precursor [Homo sapiens] | 1.60E−11 | 4502149 | 4 | 11 | 6 |
| 54 | crystallin, alpha B; heat-shock 20 kD like-protein [Homo sapiens] | 1.82E−11 | 4503057 | 6 | 6 | 2 |
| 55 | lipocalin 2 (oncogene 24p3) [Homo sapiens] | 2.38E−11 | 38455402 | 3 | 1 | 1 |
| 55 | fibrinogen, gamma chain isoform gamma-B precursor [Homo sapiens] | 3.10E−11 | 11761633 | 6 | 3 | 2 |
| 56 | apolipoprotein A-IV precursor [Homo sapiens] | 4.20E−11 | 4502151 | 4 | 6 | 3 |
| 57 | crystallin, beta B2; eye lens structural protein [Homo sapiens] | 4.24E−11 | 4503063 | 7 | 8 | 4 |
| 58 | haptoglobin [Homo sapiens] | 7.34E−11 | 4826762 | 7 | 9 | 6 |
| 59 | alpha-1-microglobulin/bikunin precursor; Alpha-1-microglobulin/bikunin | 8.33E−11 | 4502067 | 3 | 8 | 8 |
| 60 | lactotransferrin; lactoferrin [Homo sapiens] | 9.25E−11 | 4505043 | 11 | 3 | 3 |
| 61 | S-arrestin [Homo sapiens] | 9.37E−11 | 4506781 | 5 | 3 | 3 |
| 62 | pyruvate kinase 3 isoform 1; thyroid hormone-bind | 1.07E−10 | 33286418 | 2 | 3 | 3 |
| 63 | Niemann-Pick disease, type C2 precursor; epididymal | 1.08E−10 | 5453678 | 1 | 4 | 4 |
| 64 | cystatin C precursor; cystatin 3; gamma-trace; post | 1.15E−10 | 4503107 | 6 | 11 | 9 |
| 65 | chondroltin sulfate proteoglycan 2 (versican) [Hom | 1.19E−10 | 21361116 | 2 | 2 | 2 |
| 66 | crystallin, gamma C; crystallin, gamma-3 [Homo sa | 1.39E−10 | 10518338 | 3 | 3 | 1 |
| 67 | PREDICTED: similar to Von Ebners gland protein pr | 1.42E−10 | 27498839 | 2 | 3 | 3 |
| 67 | prostatic binding protein; phosphatidylethanolamine | 1.66E−10 | 4505621 | 2 | 2 | 2 |
| 68 | proline rich 4 (lacrimal); lacrimal proline rich pr | 2.37E−10 | 6005802 | 1 | 1 | 1 |
| 69 | cathepsin D preproprotein [Homo sapiens] | 2.95E−10 | 4503143 | 2 | 8 | 7 |
| 70 | RNA-binding protein regulatory subunit; oncogene | 5.62E−10 | 31543380 | 1 | 2 | 1 |
| 71 | tyrosine 3/tryptophan 5-monooxygenase activation | 5.63E−10 | 21735625 | 1 | 2 | 2 |
| 72 | glyceraldehyde-3-phosphate dehydrogenase [Homo sapiens] | 5.99E−10 | 7669492 | 3 | 5 | 5 |
| 73 | hypothetical protein BC013767 [Homo sapiens] | 8.80E−10 | 39930521 | 1 | 1 | 1 |
| 74 | ATPase, H+ transporting, lysosomal accessory prote | 8.95E−10 | 17136148 | 1 | 2 | 2 |
| 75 | coagulation factor II precursor; prothrombin [Homo | 1.03E−09 | 4503635 | 1 | 2 | 2 |
| 76 | amyloid beta (A4) precursor-like protein 2; amylol | 1.07E−09 | 4502147 | 3 | 5 | 5 |
| 77 | complement component 1 inhibitor precursor [Homo sapiens] | 1.28E−09 | 4557379 | 4 | 12 | 10 |
| 78 | filensin; cytoskeletal protein, 115 KD [Homo sapie | 1.32E−09 | 4502399 | 3 | 3 | 1 |
| 79 | prolactin-induced protein; prolactin-inducible pro | 1.65E−09 | 4505821 | 2 | 3 | 3 |
| 80 | GDP dissociation inhibitor 1; mental retardation, | 2.24E−09 | 4503971 | 1 | 1 | 1 |
| 81 | complement factor B preproprotein; C3 proactivator; C3 proaccelerator; | 2.63E−09 | 4502397 | 4 | 12 | 5 |
| 82 | apolipoprotein B precursor; apoB-100; apoB-48 [Hom | 2.74E−09 | 4502153 | 2 | 1 | 1 |
| 83 | galectin 3 binding protein; L3 antigen; Mac-2-bind | 2.94E−09 | 5031863 | 1 | 2 | 2 |
| 84 | alpha-1-antichymotrypsin, precursor; alpha-1-antichymotrypsin; antichym [symbol] | 3.02E−09 | 4501843 | 3 | 10 | 7 |
| 85 | opticin; opticin, oculoglycan; oculoglycan [Homo sapiens] | 3.15E−09 | 7657419 | 2 | 7 | 4 |
| 86 | complement component 1, r subcomponent [Homo sapie | 3.42E−09 | 4502493 | 1 | 1 | 1 |
| 87 | cystatin B; stefin B; liver thiol proteinase inhib | 4.26E−09 | 4503117 | 1 | 1 | 1 |
| 88 | pregnancy-zone protein; Pregnancy zone protein [Homo sapiens] | 4.49E−09 | 4506355 | 1 | 3 | 3 |
| 89 | haptoglobin-related protein; Haptoglobin-related locus [Homo sapiens] | 5.75E−09 | 45580723 | 7 | 9 | 6 |
| 90 | retinoschisis (X-linked, juvenile) 1 [Homo sapiens | 6.93E−09 | 10835083 | 1 | 2 | 2 |
| 91 | retinaldehyde binding protein 1; Newfoundland rod-c | 1.07E−08 | 4506541 | 1 | 1 | 1 |
| 92 | proprotein convertase subtilisin/kexin type 2; sub | 1.13E−08 | 20336244 | 1 | 2 | 2 |
| 93 | pancreatic ribonuclease precursor; RNase upl-1 [Homo sapiens] | 1.24E−08 | 38201682 | 1 | 4 | 4 |
| 94 | Fc fragment of IgG binding protein; IgG Fc binding | 1.36E−08 | 4503681 | 8 | 3 | 3 |
| 94 | inter-alpha (globulin) inhibitor H4 (plasma Kalli | 1.48E−08 | 31542984 | 2 | 1 | 1 |
| 95 | calsyntenin 1; non-classical cadherin XB31alpha1; | 1.69E−08 | 41281561 | 1 | 3 | 3 |
| 96 | alpha-2-glycoprotein 1, zinc; Alpha-2-glycoprotein, | 1.77E−08 | 4502337 | 2 | 3 | 3 |
| 97 | kininogen 1; alpha-2-thiol proteinase inhibitor; bradykinin [Homo sapie | 1.79E−08 | 4504893 | 3 | 9 | 5 |
| 98 | Wnt inhibitory factor-1 precursor; Wnt inhibitory factor-1 [Homo sapien | 2.09E−08 | 6005950 | 3 | 4 | 4 |
| 99 | tubulin alpha 6 [Homo sapiens] | 4.32E−08 | 14389309 | 1 | 3 | 3 |
| 100 | histidine-rich glycoprotein precursor; histidine-proline rich glycoprot | 5.05E−08 | 4504489 | 2 | 5 | 4 |
| 101 | amyloid beta (A4) precursor-like protein 1 [Homo sa | 5.89E−08 | 4885065 | 1 | 5 | 5 |
| 102 | vimentin [Homo sapiens] | 6.52E−08 | 4507895 | 3 | 1 | 1 |
| 103 | fibrinogen, alpha chain isoform alpha preproprotei | 7.24E−08 | 11761629 | 5 | 2 | 2 |
| 104 | SPARC-like 1; hevin; mast9 [Homo sapiens] | 9.96E−08 | 21359871 | 1 | 4 | 4 |
| 105 | cellular retinoic acid binding protein 1; cellular | 1.25E−07 | 4758052 | 2 | 1 | 1 |
| 106 | lactate dehydrogenase C [Homo sapiens] | 1.45E−07 | 4504973 | 1 | 1 | 1 |
| 107 | I factor (complement) [Homo sapiens] | 1.88E−07 | 4504579 | 2 | 8 | 6 |
| 108 | aldehyde dehydrogenase 1A1; aldehyde dehydrogenase | 2.97E−07 | 21361176 | 3 | 2 | 1 |
| 109 | heparan sulfate proteoglycan 2; heparan sulfate pr | 3.21E−07 | 7427517 | 1 | 1 | 1 |
| 109 | serine (or cysteine) proteinase inhibitor, clade C (antithrombin), memb | 3.24E−07 | 4502261 | 3 | 8 | 6 |
| 110 | triosephosphate isomerase 1 [Homo sapiens] | 3.41E−07 | 4507645 | 1 | 1 | 1 |
| 111 | heterogeneous nuclear ribonucleoprotein A1 isoform | 3.75E−07 | 14043070 | 1 | 1 | 1 |
| 112 | biotinidase precursor [Homo sapiens] | 4.83E−07 | 4557373 | 1 | 2 | 2 |
| 113 | neuronal cell adhesion molecule; Bravo [Homo sapie | 5.84E−07 | 41281389 | 1 | 1 | 1 |
| 114 | chromogranin B precursor; Chromogranin B (secretog | 7.55E−07 | 4502807 | 1 | 1 | 1 |
| 115 | myoglobin [Homo sapiens] | 9.47E−07 | 44955885 | 1 | 2 | 2 |
| 116 | semaphorin 7A; sema domain, immunoglobulin domain | 1.10E−06 | 4504237 | 2 | 2 | 2 |
| 117 | retbindin [Homo sapiens] | 1.13E−06 | 13899247 | 1 | 3 | 3 |
| 118 | phosphoglycerate kinase 2; phosphoglycerate kinas | 1.23E−06 | 31543397 | 1 | 1 | 1 |
| 119 | cystatin S precursor; cystatin 4 [Homo sapiens] | 2.01E−06 | 4503109 | 1 | 2 | 2 |
| 120 | tubulin, alpha 2 isoform 2 [Homo sapiens] | 2.17E−06 | 17921991 | 1 | 2 | 2 |
| 121 | fibrinogen, beta chain preproprotein [Homo sapiens | 2.19E−06 | 11761631 | 2 | 1 | 1 |
| 122 | H4 histone family, member E [Homo sapiens] | 2.50E−06 | 11415030 | 1 | 3 | 3 |
| 123 | complement factor H; H factor-1 (complement); fact | 2.72E−06 | 4504375 | 1 | 1 | 1 |
| 124 | immunoglobulin J chain [Homo sapiens] | 2.74E−06 | 21489959 | 1 | 1 | 1 |
| 125 | serum amyloid A2 [Homo sapiens] | 2.77E−06 | 13540475 | 2 | 2 | 2 |
| 126 | internexin neuronal intermediate filament protein, | 3.10E−06 | 14249342 | 1 | 1 | 1 |
| 127 | cellular repressor of E1A-stimulated genes [Homo sa | 3.41E−06 | 4503037 | 1 | 1 | 1 |
| 128 | insulin-like growth factor binding protein 7 [Homo | 3.61E−06 | 4504619 | 1 | 2 | 2 |
| 129 | alpha 1 type II collagen isoform 1; collagen II, a | 4.47E−06 | 13435125 | 2 | 2 | 2 |
| 130 | sparc/osteonectin, cwcv and kazal-like domains pro | 4.61E−06 | 4759164 | 1 | 3 | 3 |
| 131 | fibulin 1 isoform C precursor [Homo sapiens] | 4.72E−06 | 34734062 | 1 | 1 | 1 |
| 132 | metallothionein 1G [Homo sapiens] | 6.02E−06 | 10835230 | 1 | 2 | 2 |
| 133 | alcohol dehydrogenase 1B (class I), beta polypept | 6.39E−06 | 34577061 | 1 | 1 | 1 |
| 134 | heterogeneous nuclear ribonucleoprotein A2/B1 iso | 6.82E−06 | 14043072 | 1 | 1 | 1 |
| 135 | general transcription factor IIE, polypeptide 2, b | 7.03E−06 | 4504195 | 1 | 1 | 1 |
| 136 | PREDICTED: similar to Phosphoglycerate mutase 1 ( | 7.24E−06 | 41149700 | 1 | 2 | 2 |
| 137 | phosphoglycerate kinase 1 [Homo sapiens] | 7.80E−06 | 4505763 | 2 | 2 | 2 |
| 138 | crystallin, gamma B; crystallin, gamma 1-2 [Homo sa | 9.04E−06 | 4885157 | 1 | 1 | 1 |
| 139 | chitinase 3-like 1; cartilage glycoprotein-39 [Homo sapiens] | 9.78E−06 | 4557018 | 1 | 4 | 3 |
| 140 | KIAA0052 [Homo sapiens] | 1.24E−05 | 39930353 | 1 | 1 | 1 |
| 141 | PREDICTED: similar to Triosephosphate isomerase (T | 1.24E−05 | 41058276 | 1 | 1 | 1 |
| 142 | alpha 1 actin precursor; alpha skeletal muscle act | 1.29E−05 | 4501881 | 1 | 3 | 3 |
| 143 | aldolase C, fructose-bisphosphate; Aldolase C, fruc | 1.31E−05 | 4885063 | 1 | 1 | 1 |
| 144 | keratin 6 irs [Homo sapiens] | 1.43E−05 | 15618995 | 1 | 1 | 1 |
| 145 | ubiquitin B precursor; polyubiquitin B [Homo sapie | 1.49E−05 | 11024714 | 2 | 2 | 2 |
| 146 | fatty acid binding protein 1, liver; Fatty acid-bin | 1.51E−05 | 4557577 | 1 | 1 | 1 |
| 147 | collagen, type VI, alpha 1 precursor; collagen VI | 1.85E−05 | 15011913 | 1 | 1 | 1 |
| 148 | heterogeneous nuclear ribonucleoprotein K isoform | 2.08E−05 | 14165435 | 1 | 1 | 1 |
| 149 | heat shock 90 kDa protein 1, alpha; heat shock 90 k | 2.15E−05 | 40254816 | 1 | 1 | 1 |
| 150 | T-cell immunomodulatory protein [Homo sapiens] | 2.47E−05 | 21361933 | 1 | 1 | 1 |
| 151 | complement component 9 [Homo sapiens] | 2.66E−05 | 4502511 | 1 | 2 | 2 |
| 152 | cathepsin B preproprotein; APP secretase; preproca | 2.76E−05 | 22538431 | 1 | 2 | 2 |
| 153 | limbic system-associated membrane protein [Homo sa | 3.01E−05 | 45594240 | 1 | 1 | 1 |
| 154 | glutaredoxin (thioltransferase) [Homo sapiens] | 3.18E−05 | 4504025 | 1 | 1 | 1 |
| 155 | transketolase [Homo sapiens] | 3.22E−05 | 4507521 | 2 | 2 | 2 |
| 156 | peptidoglycan recognition protein L precursor [Homo sapiens] | 3.34E−05 | 21361845 | 1 | 3 | 3 |
| 157 | PDZ domain containing 3 isoform a; PDZ domain con | 3.34E−05 | 30061507 | 1 | 2 | 2 |
| 158 | GRB2-associated binding protein 2 isoform a; Grb2 | 3.47E−05 | 18105042 | 1 | 1 | 1 |
| 159 | PREDICTED: hypothetical protein XP_373740 [Homo sapiens] | 3.65E−05 | 41222847 | 2 | 13 | 9 |
| 160 | superoxide dismutase 3, extracellular [Homo sapien | 3.79E−05 | 4507151 | 1 | 1 | 1 |
| 161 | hypothetical protein FLJ32440 [Homo sapiens] | 3.97E−05 | 27734761 | 1 | 3 | 2 |
| 162 | pleiotrophin; heparin affin regulatory protein; hep | 4.01E−05 | 4506281 | 1 | 1 | 1 |
| 163 | A-gamma globin; hemoglobin gamma-a chain; Hemoglo | 4.02E−05 | 28302131 | 2 | 1 | 1 |
| 164 | dual specificity phosphatase 8; serine/threonine specific protein phosp | 4.25E−05 | 4758212 | 1 | 2 | 2 |
| 165 | FLJ42220 protein [Homo sapiens] | 4.37E−05 | 46409506 | 1 | 2 | 2 |
| 166 | keratin 4; Keratin-4; cytokeratin 4; keratin, typ | 4.40E−05 | 17318574 | 1 | 2 | 2 |
| 167 | vitronectin precursor; serum spreading factor; somatomedin B; compleme [symbol] | 4.77E−05 | 18201911 | 1 | 2 | 2 |
| 168 | chondromodulin I precursor; chondromodulin; BRICHOS | 4.77E−05 | 5901932 | 1 | 2 | 2 |
| 169 | keratin 1; Keratin-1; cytokeratin 1; hair alpha pr | 5.28E−05 | 17318569 | 2 | 1 | 1 |
| 170 | PREDICTED: similar to phosducin-like 3; phosducin-like 2; IAP-associat | 5.29E−05 | 51492730 | 3 | 4 | 2 |
| 171 | cadherin 2, type 1 preproprotein; cadherin 2, N-c | 5.73E−05 | 14589889 | 1 | 1 | 1 |
| 172 | lysosomal trafficking regulator; beige protein [Hom | 5.78E−05 | 4502839 | 1 | 4 | 4 |
| 173 | PREDICTED: cardiomyopathy associated 3 [Homo sapiens] | 5.89E−05 | 51460893 | 1 | 4 | 2 |
| 174 | hypothetical protein MGC10882 [Homo sapiens] | 6.22E−05 | 14249240 | 1 | 1 | 1 |
| 175 | amyloid beta A4 protein precursor, isoform b; pro | 6.60E−05 | 41406055 | 1 | 3 | 3 |
| 176 | PREDICTED: hypothetical protein XP_376099 [Homo sapiens] | 6.72E−05 | 42656388 | 1 | 4 | 3 |
| 177 | sodium-dependent organic anion transporter [Homo sapiens] | 6.81E−05 | 37537552 | 1 | 1 | 1 |
| 178 | tubulin, alpha 4 [Homo sapiens] | 6.95E−05 | 13376539 | 1 | 1 | 1 |
| 179 | oral-facial-digital syndrome 1; chromosome X open | 7.51E−05 | 4503179 | 1 | 1 | 1 |
| 180 | ribosomal protein S4, Y-linked 1 Y isoform; 40S ribosomal protein S4, Y | 8.87E−05 | 4506727 | 1 | 1 | 1 |
| 181 | PREDICTED: zinc finger, CCHC domain containing 11 | 9.18E−05 | 41107445 | 1 | 2 | 1 |
| 182 | calbindin 2 full length protein isoform; calbindin | 9.84E−05 | 4502543 | 1 | 1 | 1 |
| 183 | hypothetical protein MGC34732 [Homo sapiens] | 9.92E−05 | 40255180 | 1 | 1 | 1 |
| 184 | dual specificity phosphatase 5; serine/threonine specific protein phos | 1.12E−04 | 12707566 | 1 | 5 | 4 |
| 185 | apolipoprotein C-III precursor [Homo sapiens] | 1.13E−04 | 4557323 | 1 | 1 | 1 |
| 186 | prosaposin (variant Gaucher disease and variant metachromatic leukodys [symbol] | 1.13E−04 | 11386147 | 1 | 2 | 2 |
| 187 | defensin, alpha 1 preproprotein; myeloid-related s | 1.30E−04 | 4758146 | 1 | 2 | 2 |
| 188 | complement factor D preproprotein; adipsin; prope | 1.32E−04 | 42544239 | 1 | 1 | 1 |
| 189 | hypothetical protein FLJ25369 [Homo sapiens] | 1.38E−04 | 22749357 | 1 | 1 | 1 |
| 190 | PREDICTED: similar to RIKEN cDNA 1700019P01 [Homo | 1.39E−04 | 51476104 | 1 | 1 | 1 |
| 191 | lumican [Homo sapiens] | 1.40E−04 | 4505047 | 1 | 1 | 1 |
| 192 | ubiquitin carboxyl-terminal esterase L1 (ubiquiti | 1.47E−04 | 21361091 | 1 | 1 | 1 |
| 193 | nucleoporin 214 kDa; nuclear pore complex protein | 1.49E−04 | 33946327 | 1 | 3 | 3 |
| 194 | importin 13; Ran binding protein 13; karyopherin 13 [Homo sapiens] | 1.49E−04 | 41281425 | 1 | 1 | 1 |
| 195 | proprotein convertase subtilisin/kexin type 5 preproprotein; protease | 1.54E−04 | 20336246 | 1 | 1 | 1 |
| 196 | ankyrin repeat and SOCS box-containing protein 12 | 1.55E−04 | 22208955 | 2 | 1 | 1 |
| 197 | heterogeneous nuclear ribonucleoprotein C isoform | 1.55E−04 | 14110428 | 1 | 1 | 1 |
| 198 | hypothetical protein FLJ10350 [Homo sapiens] | 1.61E−04 | 21361781 | 1 | 1 | 1 |
| 199 | family with sequence similarity 29, member A [Homo sapiens] | 1.71E−04 | 31377562 | 1 | 1 | 1 |
| 200 | acyl-Coenzyme A dehydrogenase, very long chain pre | 1.84E−04 | 4557235 | 2 | 3 | 3 |
| 201 | PREDICTED: similar to Putative dimethylaniline mo | 1.85E−04 | 51458831 | 1 | 1 | 1 |
| 202 | TATA box-binding protein-associated factor 2F; TA | 1.97E−04 | 14717407 | 1 | 1 | 1 |
| 203 | enolase 1; MYC promoter-binding protein 1; non-neu | 2.01E−04 | 4503571 | 1 | 1 | 1 |
| 204 | phosphodiesterase 6A, alpha subunit [Homo sapiens] | 2.08E−04 | 4585864 | 1 | 6 | 5 |
| 205 | plasminogen [Homo sapiens] | 2.12E−04 | 4505881 | 1 | 3 | 2 |
| 206 | dual oxidase 2 precursor; dual oxidase-like domains 2; nicotinamide ad | 2.36E−04 | 28872753 | 1 | 4 | 4 |
| 207 | sperm associated antigen 6 isoform 2; sperm flagellar protein; axoneme | 2.37E−04 | 27262641 | 2 | 2 | 1 |
| 208 | spondin 1, extracellular matrix protein; spondin | 2.44E−04 | 24307905 | 1 | 2 | 2 |
| 209 | hypothetical protein LOC92912 [Homo sapiens] | 2.50E−04 | 29789401 | 1 | 2 | 2 |
| 210 | CD14 antigen precursor [Homo sapiens] | 2.50E−04 | 4557417 | 1 | 1 | 1 |
| 211 | PH domain-containing protein [Homo sapiens] | 2.83E−04 | 33457316 | 1 | 2 | 2 |
| 212 | tuberous sclerosis 2 isoform 2; tuberin isoform 1; tuberin isoform 2; | 2.96E−04 | 10938008 | 1 | 1 | 1 |
| 213 | low density lipoprotein-related protein 2; megalin | 3.07E−04 | 6806919 | 1 | 1 | 1 |
| 214 | hypothetical protein FLJ20574 [Homo sapiens] | 3.10E−04 | 8923539 | 1 | 1 | 1 |
| 215 | kallikrein 14 preproprotein; kallikrein-like protein 6 [Homo sapiens] | 3.25E−04 | 11545747 | 1 | 2 | 1 |
| 216 | skeletal muscle specific actinin, alpha 3 [Homo sapiens] | 3.34E−04 | 4557241 | 1 | 4 | 3 |
| 217 | F-box protein 40; muscle disease-related protein [H | 3.51E−04 | 7706493 | 1 | 1 | 1 |
| 218 | transient receptor potential cation channel, subfamily M, member 8 [Ho | 3.58E−04 | 21361691 | 1 | 1 | 1 |
| 219 | PREDICTED: KIAA0368 protein [Homo sapiens] | 3.74E−04 | 51467589 | 1 | 1 | 1 |
| 220 | alpha-2-HS-glycoprotein; Alpha-2HS-glycoprotein [Homo sapiens] | 3.79E−04 | 4502005 | 1 | 3 | 3 |
| 221 | PREDICTED: similar to KIAA0663 gene product [Homo | 3.85E−04 | 41058123 | 1 | 3 | 3 |
| 222 | guanine nucleotide-binding protein, beta-4 subuni | 4.03E−04 | 11055998 | 1 | 1 | 1 |
| 223 | H2A histone family, member A; histone H2AE [Homo | 4.17E−04 | 10645195 | 1 | 2 | 2 |
| 224 | potassium channel, subfamily K, member 12; tandem pore domain potassiu | 4.26E−04 | 11545761 | 1 | 1 | 1 |
| 225 | milk fat globule-EGF factor 8 protein; lactadherin | 4.40E−04 | 5174557 | 1 | 1 | 1 |
| 226 | Kruppel-like factor 12 isoform a; KLF12 zinc fing | 4.42E−04 | 21071074 | 1 | 1 | 1 |
| 227 | PREDICTED: similar to sin3 associated polypeptide p18 [Homo sapiens] | 4.51E−04 | 51461003 | 1 | 3 | 3 |
| 227 | sushi-repeat-containing protein, X-linked 2; sushi | 4.60E−04 | 7657619 | 1 | 1 | 1 |
| 228 | galactosylceramidase precursor; galactocerebrosidase; Galactosylceramin | 4.63E−04 | 4557613 | 1 | 1 | 1 |
| 229 | heat shock protein apg-1 [Homo sapiens] | 4.66E−04 | 31541941 | 1 | 1 | 1 |
| 230 | PAP associated domain containing 1 [Homo sapiens] | 4.69E−04 | 21361704 | 1 | 1 | 1 |
| 231 | PREDICTED: hypothetical protein FLJ25756 [Homo sapiens] | 4.76E−04 | 51472397 | 2 | 4 | 4 |
| 232 | zinc finger protein 262; cell death inhibiting RN | 4.80E−04 | 44890068 | 1 | 1 | 1 |
| 233 | golgi autoantigen, golgin subfamily b, macrogolgin (with transmembrane | 5.05E−04 | 4758454 | 1 | 2 | 2 |
| 234 | notch4 preproprotein; Notch (Drosophila) homolog 4 [Homo sapiens] | 5.09E−04 | 27894370 | 1 | 1 | 1 |
| 235 | protocadherin gamma subfamily B, 2 isoform 1 prec | 5.25E−04 | 11128035 | 1 | 1 | 1 |
| 236 | PREDICTED: similar to ribosomal protein L31 [Homo sapiens] | 5.33E−04 | 51466239 | 1 | 2 | 2 |
| 237 | insulin-like growth factor binding protein 6 [Homo | 5.46E−04 | 11321593 | 1 | 2 | 2 |
| 238 | semaphorin 3D; collapsin 2 [Homo sapiens] | 5.79E−04 | 41406086 | 1 | 2 | 2 |
| 239 | hypothetical protein MGC5601; similar to acyl-CoA dehydrogenase [Homo | 5.87E−04 | 31543201 | 1 | 1 | 1 |
| 240 | solute carrier family 37 (glycerol-3-phosphate tra | 5.94E−04 | 14150047 | 1 | 1 | 1 |
| 241 | plexin B3; plexin-B3; plexin 6 [Homo sapiens] | 5.99E−04 | 29336063 | 1 | 3 | 1 |
| 242 | PREDICTED: KIAA1030 protein [Homo sapiens] | 6.08E−04 | 42659643 | 1 | 3 | 3 |
| 243 | CP110 protein [Homo sapiens] | 6.19E−04 | 31543018 | 2 | 3 | 3 |
| 244 | core-binding factor, beta subunit isoform 2; SL3/ | 6.19E−04 | 13124873 | 1 | 1 | 1 |
| 245 | PREDICTED: agrin [Homo sapiens] | 6.20E−04 | 41151826 | 1 | 1 | 1 |
| 246 | lacritin precursor [Homo sapiens] | 6.51E−04 | 15187164 | 1 | 2 | 2 |
| 247 | brain expressed, X-linked 1; uncharacterized hypo | 6.54E−04 | 15147228 | 1 | 1 | 1 |
| 248 | PREDICTED: similar to 60S ribosomal protein L23a [Homo sapiens] | 6.71E−04 | 51474226 | 1 | 1 | 1 |
| 249 | ubiquitin specific protease 36 [Homo sapiens] | 6.71E−04 | 35250686 | 1 | 1 | 1 |
| 250 | ubiquitin specific protease 15; deubiquitinating enzyme [Homo sapiens] | 6.90E−04 | 14149627 | 1 | 3 | 2 |
| 251 | coatomer protein complex, subunit gamma 2; coat pr | 7.04E−04 | 6912320 | 1 | 2 | 2 |
| 252 | PREDICTED: similar to hect domain and RLD 2 [Homo | 7.22E−04 | 51472707 | 1 | 2 | 2 |
| 253 | putatative 28 kDa protein [Homo sapiens] | 7.35E−04 | 10047140 | 1 | 1 | 1 |
| 254 | metallothionein 3 [Homo sapiens] | 7.43E−04 | 5174762 | 1 | 1 | 1 |
| 255 | PREDICTED: similar to Piccolo protein (Aczonin) [Homo sapiens] | 7.55E−04 | 51492882 | 1 | 2 | 2 |
| 256 | hypothetical protein DKFZp434H0115 [Homo sapiens] | 7.76E−04 | 13899233 | 1 | 1 | 1 |
| 257 | hypothetical protein DKFZp434O0527 [Homo sapiens] | 7.81E−04 | 34916026 | 1 | 5 | 4 |
| 258 | PREDICTED: KIAA1604 protein [Homo sapiens] | 8.01E−04 | 20536216 | 1 | 1 | 1 |
| 259 | enhancer of zeste 2 isoform a; enhancer of zeste | 8.14E−04 | 21361095 | 1 | 1 | 1 |
| 260 | hypothetical protein FLJ20580 [Homo sapiens] | 8.20E−04 | 8923541 | 1 | 4 | 3 |
| 261 | complement component 2 precursor; C3/C5 convertase [Homo sapiens] | 8.23E−04 | 14550407 | 1 | 1 | 1 |
| 262 | SH3 and multiple ankyrin repeat domains 1; somatostatin receptor-inter | 8.27E−04 | 11968152 | 1 | 1 | 1 |
| 263 | sparc/osteonectin, cwcv and kazal-like domains prot | 8.35E−04 | 7662036 | 1 | 1 | 1 |
| 264 | PREDICTED: KIAA0882 protein [Homo sapiens] | 8.39E−04 | 22042713 | 1 | 1 | 1 |
| 265 | propionyl-Coenzyme A carboxylase, alpha polypeptid | 8.42E−04 | 4557833 | 1 | 1 | 1 |
| 266 | PREDICTED: KIAA0179 [Homo sapiens] | 8.94E−04 | 51475296 | 1 | 4 | 4 |
| 267 | myosin IIIA; deafness, autosomal recessive 30 [Homo sapiens] | 9.51E−04 | 24586657 | 1 | 1 | 1 |
| 268 | zinc finger protein 606 [Homo sapiens] | 9.52E−04 | 31377839 | 1 | 1 | 1 |
| 269 | PREDICTED: hypothetical protein LOC146177 [Homo s | 9.52E−04 | 51472829 | 1 | 2 | 2 |
| 270 | SNF2 histone linker PHD RING helicase; 2610103K11 | 9.92E−04 | 27436873 | 1 | 2 | 2 |
| 271 | pantothenate kinase 3; pantothenic acid kinase [Homo sapiens] | 1.01E−03 | 13375789 | 1 | 3 | 2 |
| 272 | protein tyrosine phosphatase, receptor type, K pr | 1.04E−03 | 18860902 | 1 | 1 | 1 |
| 273 | elongation of very long chain fatty acids like 3 [Homo sapiens] | 1.06E−03 | 23097310 | 1 | 1 | 1 |
| 274 | hypothetical protein MGC35261 [Homo sapiens] | 1.07E−03 | 33859793 | 1 | 1 | 1 |
| 275 | GTPase regulator associated with the focal adhesion kinase pp125; GTPas | 1.08E−03 | 7662208 | 1 | 6 | 4 |
| 276 | cleavage and polyadenylation specific factor 3, 73 kDa; cleavage and pol | 1.10E−03 | 7706427 | 1 | 3 | 2 |
| 277 | PREDICTED: KIAA0543 protein [Homo sapiens] | 1.13E−03 | 51466599 | 1 | 1 | 1 |
| 278 | serine (or cysteine) proteinase inhibitor, clade I | 1.14E−03 | 4826904 | 1 | 1 | 1 |
| 279 | Tax1 (human T-cell leukemia virus type I) binding protein 1; tax1-bind | 1.14E−03 | 21361682 | 1 | 2 | 2 |
| 280 | PREDICTED: similar to KIAA0877 protein [Homo sapie | 1.15E−03 | 51466121 | 1 | 1 | 1 |
| 280 | hypothetical protein FLJ12998 [Homo sapiens] | 1.16E−03 | 12232437 | 1 | 1 | 1 |
| 281 | PREDICTED: intracellular membrane-associated calc | 1.19E−03 | 37538661 | 1 | 1 | 1 |
| 282 | PREDICTED: KIAA0367 protein [Homo sapiens] | 1.19E−03 | 51467193 | 1 | 3 | 3 |
| 283 | PREDICTED: similar to Heat shock cognate 71 kDa pr | 1.20E−03 | 51466978 | 1 | 1 | 1 |
| 284 | LIM domain only 7; LOMP protein; zinc-finger doma | 1.21E−03 | 33598968 | 1 | 1 | 1 |
| 285 | centromere protein C 1; Centromere autoantigen C1 [Homo sapiens] | 1.23E−03 | 4502779 | 1 | 2 | 2 |
| 286 | cytoplasmic FMR1 interacting protein 1; selective hybridizing clone (m | 1.25E−03 | 24307969 | 1 | 1 | 1 |
| 287 | synuclein, gamma (breast cancer-specific protein 1) | 1.25E−03 | 4507113 | 1 | 1 | 1 |
| 288 | aldo-keto reductase family 1, member C3; hydroxys | 1.28E−03 | 24497583 | 1 | 1 | 1 |
| 289 | formin-like 3 isoform 1; WW domain binding protei | 1.28E−03 | 40217839 | 1 | 1 | 1 |
| 290 | hypothetical protein FLJ31322 [Homo sapiens] | 1.29E−03 | 45387953 | 1 | 2 | 1 |
| 291 | dynein, axonemal, intermediate polypeptide 2; dyn | 1.31E−03 | 12718866 | 1 | 2 | 2 |
| 292 | brain glycogen phosphorylase; glycogen phosphoryl | 1.32E−03 | 21361370 | 1 | 1 | 1 |
| 293 | solute carrier family 4, sodium bicarbonate cotra | 1.33E−03 | 19923176 | 1 | 1 | 1 |
| 294 | FLJ46156 protein [Homo sapiens] | 1.33E−03 | 38348312 | 1 | 1 | 1 |
| 295 | corticosteroid binding globulin precursor; corticosteroid binding globu | 1.35E−03 | 4502595 | 1 | 1 | 1 |
| 296 | PREDICTED: similar to anaphase promoting complex | 1.36E−03 | 51460657 | 1 | 1 | 1 |
| 297 | PREDICTED: hypothetical protein XP_498867 [Homo sapiens] | 1.37E−03 | 51475312 | 1 | 2 | 2 |
| 298 | PREDICTED: similar to tudor domain containing 6 protein [Homo sapiens] | 1.40E−03 | 42657656 | 1 | 1 | 1 |
| 299 | PREDICTED: hypothetical cardiac/skeletal muscle-e | 1.41E−03 | 51458828 | 1 | 1 | 1 |
| 300 | zinc finger protein 560 [Homo sapiens] | 1.43E−03 | 22749003 | 1 | 3 | 2 |
| 301 | bromodomain adjacent to zinc finger domain, 1A isoform a; ATP-dependen | 1.48E−03 | 32967603 | 1 | 1 | 1 |
| 302 | hypothetical protein MGC27016 [Homo sapiens] | 1.48E−03 | 21450675 | 1 | 2 | 1 |
| 303 | ribosomal protein L10-like protein [Homo sapiens] | 1.50E−03 | 18152783 | 1 | 4 | 2 |
| 304 | RAN binding protein 2; nucleoporin 358; nuclear po | 1.51E−03 | 6382079 | 1 | 1 | 1 |
| 305 | PREDICTED: hypothetical protein XP_098980 [Homo s | 1.51E−03 | 51477357 | 1 | 1 | 1 |
| 306 | supervillin isoform 2; membrane-associated F-actin binding protein p20 | 1.51E−03 | 11496982 | 1 | 12 | 9 |
| 307 | H63 breast cancer expressed gene isoform a; gene associated with HER-2 | 1.59E−03 | 29826289 | 1 | 1 | 1 |
| 308 | alpha 1 type XV collagen precursor; collagen XV, | 1.61E−03 | 18641350 | 1 | 1 | 1 |
| 309 | beta-1,3-N-acetylglucosaminyltransferase bGnT-6; i | 1.65E−03 | 5802984 | 1 | 1 | 1 |
| 310 | RAN binding protein 3 isoform RANBP3-a; RAN-bindin | 1.69E−03 | 4506409 | 1 | 1 | 1 |
| 311 | zinc finger protein 335; zinc-finger/leucine-zipp | 1.73E−03 | 11560152 | 1 | 1 | 1 |
| 312 | PREDICTED: hypothetical protein XP_370924 [Homo s | 1.74E−03 | 51473154 | 1 | 1 | 1 |
| 313 | mitogen-activated protein kinase associated prote | 1.75E−03 | 13129138 | 1 | 2 | 2 |
| 314 | PREDICTED: hypothetical protein LOC284371 [Homo s | 1.79E−03 | 51474776 | 1 | 1 | 1 |
| 315 | stromal cell-derived factor 2 precursor [Homo sapi | 1.79E−03 | 14141195 | 1 | 1 | 1 |
| 316 | frizzled-related protein; Fritz; Frzb-1; fre; friz | 1.81E−03 | 38455388 | 1 | 2 | 2 |
| 317 | desmoplakin; desmoplakin (DPI, DPII) [Homo sapiens | 1.81E−03 | 4758200 | 1 | 1 | 1 |
| 318 | TU12B1-TY protein [Homo sapiens] | 1.82E−03 | 7706749 | 1 | 1 | 1 |
| 319 | biliverdin reductase A [Homo sapiens] | 1.86E−03 | 33589854 | 1 | 1 | 1 |
| 320 | PREDICTED: similar to Ig kappa variable region [Ho | 1.87E−03 | 51460659 | 1 | 1 | 1 |
| 320 | SH3-domain binding protein 2; Cherubism [Homo sap | 1.87E−03 | 19923155 | 1 | 2 | 2 |
| 321 | U5 snRNP-specific protein, 116 kD [Homo sapiens] | 1.88E−03 | 41152056 | 1 | 1 | 1 |
| 322 | PREDICTED: similar to Ig kappa chain [Homo sapien | 1.91E−03 | 51475407 | 1 | 1 | 1 |
| 323 | formyltetrahydrofolate dehydrogenase isoform a [H | 1.91E−03 | 21614513 | 1 | 1 | 1 |
| 324 | rotatin [Homo sapiens] | 1.91E−03 | 38201696 | 1 | 1 | 1 |
| 325 | hypothetical protein FLJ38973 [Homo sapiens] | 1.93E−03 | 31581541 | 1 | 2 | 2 |
| 326 | PREDICTED: KIAA0753 gene product [Homo sapiens] | 1.96E−03 | 42661078 | 1 | 1 | 1 |
| 327 | hypothetical protein DKFZp434H2215 [Homo sapiens] | 1.96E−03 | 8922138 | 1 | 1 | 1 |
| 328 | KIAA0317 [Homo sapiens] | 1.98E−03 | 42734315 | 1 | 3 | 2 |
| 329 | PREDICTED: YLP motif containing 1 [Homo sapiens] | 2.00E−03 | 51493127 | 1 | 1 | 1 |
| 330 | ankyrin repeat and SOCS box-containing 6 isoform 1 [Homo sapiens] | 2.02E−03 | 8923516 | 1 | 1 | 1 |
| 331 | PREDICTED: KIAA1414 protein [Homo sapiens] | 2.04E−03 | 51460558 | 1 | 4 | 3 |
| 332 | T-box 21; T-box expressed in T cells; T-cell-specific T-box transcripti | 2.05E−03 | 7019549 | 1 | 1 | 1 |
| 333 | PREDICTED: similar to RIKEN cDNA 1700013E18 [Homo | 2.07E−03 | 51458576 | 1 | 1 | 1 |
| 334 | similar to hypothetical protein FLJ10883 [Homo sa | 2.09E−03 | 24308386 | 1 | 1 | 1 |
| 335 | dihydropyrimidinase-like 2; collapsin response medi | 2.11E−03 | 4503377 | 1 | 1 | 1 |
| 336 | KIAA0433 protein [Homo sapiens] | 2.12E−03 | 41281583 | 1 | 1 | 1 |
| 337 | transcription factor-like protein 4 isoform gamma; MAX-like bHLHZIP pr | 2.12E−03 | 24586667 | 1 | 8 | 5 |
| 337 | keratin, hair, basic, 4; hard keratin, type II, 4 | 2.14E−03 | 15431316 | 1 | 1 | 1 |
| 338 | fibronectin 1 isoform 3 preproprotein; cold-insol | 2.17E−03 | 16933542 | 1 | 1 | 1 |
| 339 | homeobox protein A5; homeobox protein HOXA5; home | 2.18E−03 | 24497517 | 1 | 1 | 1 |
| 340 | bromodomain containing protein 3; open reading fr | 2.19E−03 | 11067749 | 1 | 1 | 1 |
| 341 | homeobox protein A10 isoform b; homeobox protein H | 2.19E−03 | 24497551 | 1 | 1 | 1 |
| 342 | PREDICTED: GCN1 general control of amino-acid synthesis 1-like 1 [Homo | 2.23E−03 | 51471344 | 1 | 1 | 1 |
| 343 | calsyntenin 3; alcadein [Homo sapiens] | 2.23E−03 | 42475534 | 1 | 1 | 1 |
| 344 | serine/threonine kinase 36 (fused homolog, Drosophila); serine/threoni | 2.26E−03 | 24308123 | 1 | 1 | 1 |
| 345 | interphotoreceptor matrix proteoglycan 2; interpho | 2.29E−03 | 7706717 | 1 | 1 | 1 |
| 345 | zinc finger, MYND domain containing 11 isoform b; adenovirus 5 E1A bin | 2.30E−03 | 47078243 | 1 | 2 | 2 |
| 346 | Rho GTPase activating protein 24 [Homo sapiens] | 2.35E−03 | 13775230 | 1 | 1 | 1 |
| 347 | Bardet-Biedl syndrome 1 [Homo sapiens] | 2.36E−03 | 28395045 | 1 | 1 | 1 |
| 348 | serine (or cysteine) proteinase inhibitor, clade | 2.37E−03 | 13489087 | 1 | 1 | 1 |
| 349 | similar to RIKEN cDNA 1110018M03 [Homo sapiens] | 2.37E−03 | 42766422 | 1 | 2 | 2 |
| 350 | HECT domain containing 1 [Homo sapiens] | 2.40E−03 | 32698702 | 1 | 1 | 1 |
| 351 | steroid dehydrogenase-like [Homo sapiens] | 2.41E−03 | 24432037 | 1 | 1 | 1 |
| 352 | potassium voltage-gated channel, shaker-related subfamily, member 1 [Ho | 2.42E−03 | 4557685 | 1 | 1 | 1 |
| 353 | junctophilin 1; mitsugumin72; junctophilin type1 [Homo sapiens] | 2.43E−03 | 21735575 | 1 | 3 | 2 |
| 354 | kelch-like 8 [Homo sapiens] | 2.43E−03 | 34101268 | 1 | 1 | 1 |
| 355 | PREDICTED: similar to transcription elongation fa | 2.45E−03 | 51467523 | 1 | 3 | 3 |
| 356 | crumbs homolog 1 isoform II precursor; crumbs (Dr | 2.45E−03 | 41327708 | 1 | 1 | 1 |
| 357 | galectin 8 isoform a; Po66 carbohydrate binding p | 2.45E−03 | 42544185 | 1 | 1 | 1 |
| 357 | arginyl-tRNA synthetase [Homo sapiens] | 2.46E−03 | 15149476 | 1 | 4 | 4 |
| 358 | SET and MYND domain containing 1; CD8 beta opposi | 2.46E−03 | 38093643 | 1 | 1 | 1 |
| 359 | PREDICTED: similar to 40S ribosomal protein S7 (S8) [Homo sapiens] | 2.47E−03 | 51459063 | 1 | 1 | 1 |
| 360 | MAS-related GPR, member D; mas-related G protein-coupled MRGD | 2.48E−03 | 42794265 | 1 | 1 | 1 |
| | [Homo sa [symbol] | | | | | |
| 361 | hypothetical protein FLJ35725 [Homo sapiens] | 2.51E−03 | 22749135 | 1 | 1 | 1 |
| 362 | hypothetical protein FLJ21918 [Homo sapiens] | 2.54E−03 | 45935393 | 1 | 1 | 1 |
| 363 | structural maintenance of chromosomes 2-like 1; structural maintenance | 2.58E−03 | 5453591 | 1 | 1 | 1 |
| 364 | PREDICTED: hypothetical protein LOC285335 [Homo sapiens] | 2.58E−03 | 41146536 | 1 | 1 | 1 |
| 365 | PREDICTED: similar to elongation factor 1 delta [ | 2.64E−03 | 51459015 | 1 | 1 | 1 |
| 366 | leucine zipper, putative tumor suppressor 1; F37/E | 2.64E−03 | 10440566 | 1 | 3 | 2 |
| 367 | PREDICTED: similar to FLJ14624 protein [Homo sapi | 2.65E−03 | 51464636 | 1 | 1 | 1 |
| 368 | cell adhesion molecule with homology to L1CAM pre | 2.66E−03 | 27894376 | 1 | 1 | 1 |
| 369 | NFAT activation molecule 1 precursor; calcinerin/ | 2.68E−03 | 22095335 | 1 | 1 | 1 |
| 370 | B-cell lymphoma protein 2 beta isoform [Homo sapie | 2.71E−03 | 4557357 | 1 | 1 | 1 |
| 371 | cytosolic ovarian carcinoma antigen 1 isoform b; APK1 antigen [Homo sa | 2.72E−03 | 32528291 | 1 | 2 | 2 |
| 372 | hypothetical protein LOC255189 [Homo sapiens] | 2.73E−03 | 47106065 | 1 | 2 | 1 |
| 373 | postreplication repair protein hRAD18p; RAD18, S. cerevisiae, homolog | 2.76E−03 | 14550405 | 1 | 1 | 1 |
| 374 | PREDICTED: hypothetical protein XP_499235 [Homo s | 2.79E−03 | 51466834 | 1 | 1 | 1 |
| 375 | DEAH (Asp-Glu-Ala-His) box polypeptide 29; nuclei | 2.79E−03 | 26553432 | 1 | 3 | 3 |
| 376 | sodium channel, voltage-gated, type II, alpha 2; s | 2.80E−03 | 10337597 | 1 | 1 | 1 |
| 377 | nucleoporin 210; nuclear pore membrane glycoprotein 210 [Homo sapiens] | 2.84E−03 | 27477134 | 1 | 3 | 3 |
| 378 | four and a half LIM domains 1; Four-and-a-half LI | 2.85E−03 | 21361122 | 1 | 1 | 1 |
| 379 | toll-like receptor 8 isoform 1 [Homo sapiens] | 2.90E−03 | 20302166 | 1 | 1 | 1 |
| 380 | golgi autoantigen, golgin subfamily a, 4; golgin-2 | 2.90E−03 | 6715600 | 1 | 1 | 1 |
| 381 | inter-alpha (globulin) inhibitor H2; inter-alpha ( | 2.91E−03 | 4504783 | 1 | 1 | 1 |
| 382 | hypothetical protein FLJ12178 [Homo sapiens] | 2.93E−03 | 38202219 | 1 | 1 | 1 |
| 383 | a disintegrin and metalloproteinase with thrombos | 2.96E−03 | 33624885 | 1 | 1 | 1 |
| 384 | PREDICTED: hypothetical protein XP_497598 [Homo s | 2.97E−03 | 51474534 | 1 | 2 | 2 |
| 385 | lactate dehydrogenase A [Homo sapiens] | 2.98E−03 | 5031857 | 1 | 1 | 1 |
| 386 | glutathione S-transferase M1 isoform 1; HB subuni | 2.98E−03 | 23065544 | 1 | 1 | 1 |
| 387 | phospholipase C gamma 1 isoform b; phospholipase | 3.00E−03 | 33598946 | 1 | 1 | 1 |
| 388 | PREDICTED: similar to bA203I16.1 (KIAA0970 protei | 3.10E−03 | 51477225 | 1 | 1 | 1 |
| 389 | acetyl-Coenzyme A acyltransferase 1; peroxisomal 3 | 3.11E−03 | 4501853 | 1 | 1 | 1 |
| 390 | Insulin receptor substrate 1 [Homo sapiens] | 3.11E−03 | 5031805 | 1 | 3 | 1 |
| 391 | PREDICTED: similar to Tetratricopeptide repeat pr | 3.15E−03 | 29735853 | 1 | 1 | 1 |
| 392 | ankyrin repeat domain 30A; breast cancer antigen NY-BR-1 [Homo sapiens | 3.16E−03 | 16506285 | 1 | 2 | 2 |
| 393 | DEAH (Asp-Glu-Ala-His) box polypeptide 30 isoform | 3.17E−03 | 20336290 | 1 | 1 | 1 |
| 394 | SATB family member 2; two cut domains-containing | 3.24E−03 | 38016202 | 1 | 1 | 1 |
| 395 | hepatoma-derived growth factor, related protein 3; hepatoma-derived gro | 3.24E−03 | 7705320 | 1 | 1 | 1 |
| 396 | enolase 2; neurone-specific enolase; neuron specifi | 3.25E−03 | 5803011 | 1 | 1 | 1 |
| 397 | Ral GEF with PH domain and SH3 binding motif 2; Ral-A exchange factor | 3.25E−03 | 32441283 | 1 | 1 | 1 |
| 398 | PREDICTED: hypothetical protein XP_370638 [Homo s | 3.28E−03 | 41149556 | 1 | 1 | 1 |
| 399 | mitogen-activated protein kinase-activated protei | 3.28E−03 | 10863901 | 1 | 1 | 1 |
| 400 | cholinergic receptor, nicotinic, alpha polypeptide 2 (neuronal) [Homo s | 3.30E−03 | 4502823 | 1 | 1 | 1 |
| 401 | growth factor receptor-bound protein 14 [Homo sapiens] | 3.32E−03 | 4758478 | 1 | 2 | 2 |
| 402 | PREDICTED: hypothetical protein XP_295058 [Homo s | 3.34E−03 | 51460438 | 1 | 1 | 1 |
| 403 | aldehyde dehydrogenase 3 family, member A1; aldehy | 3.34E−03 | 22907049 | 1 | 1 | 1 |
| 404 | eukaryotic translation initiation factor 2C, 3 isoform a; argonaute 3 | 3.35E−03 | 29294647 | 1 | 2 | 2 |
| 405 | DnaJ (Hsp40) homolog, subfamily C, member 1 [Homo sapiens] | 3.37E−03 | 21361912 | 1 | 1 | 1 |
| 406 | ATPase, Class II, type 9B; ATPase type IV, phosph | 3.40E−03 | 41327760 | 1 | 1 | 1 |
| 407 | hypothetical protein FLJ12875 [Homo sapiens] | 3.42E−03 | 13375705 | 1 | 1 | 1 |
| 408 | PREDICTED: potassium channel, subfamily T, member | 3.46E−03 | 51467170 | 1 | 1 | 1 |
| 409 | PREDICTED: hypothetical protein XP_498937 [Homo sapiens] | 3.47E−03 | 51463864 | 1 | 1 | 1 |
| 410 | vacuolar protein sorting 13C protein [Homo sapiens] | 3.48E−03 | 42544121 | 1 | 1 | 1 |
| 411 | GREB1 protein isoform a; gene regulated by estrog | 3.50E−03 | 23397642 | 1 | 1 | 1 |
| 412 | DC12 protein [Homo sapiens] | 3.52E−03 | 9910182 | 1 | 1 | 1 |
| 413 | follistatin-like 1 precursor; follistatin-related p | 3.56E−03 | 5901956 | 1 | 1 | 1 |
| 414 | chloride channel 6 isoform CIC-6a [Homo sapiens] | 3.56E−03 | 4502873 | 1 | 1 | 1 |
| 415 | PREDICTED: hypothetical protein XP_375631 [Homo sa | 3.57E−03 | 42661664 | 1 | 2 | 2 |
| 416 | KIAA1404 protein [Homo sapiens] | 3.58E−03 | 28626521 | 1 | 1 | 1 |
| 417 | fragile X mental retardation-related protein 1; Fragile X mental retard | 3.60E−03 | 4826736 | 1 | 1 | 1 |
| 418 | BPY2 interacting protein 1; chromosome 19 open re | 3.61E−03 | 21361668 | 1 | 1 | 1 |
| 419 | vacuolar protein sorting 18 [Homo sapiens] | 3.62E−03 | 17978485 | 1 | 2 | 2 |
| 420 | phosphatidylinositol-4-phosphate 5-kinase, type I, gamma; phosphatidyl | 3.65E−03 | 31317309 | 1 | 2 | 2 |
| 421 | latent transforming growth factor beta binding pr | 3.69E−03 | 46249412 | 1 | 1 | 1 |
| 422 | transformation/transcription domain-associated pro | 3.71E−03 | 4507691 | 1 | 1 | 1 |
| 423 | neogenin homolog 1; neogenin (chicken) homolog 1 [Homo sapiens] | 3.72E−03 | 4505375 | 1 | 2 | 2 |
| 424 | Y chromosome chromodomain protein 1 isoform a; te | 3.74E−03 | 25453481 | 1 | 1 | 1 |
| 425 | PREDICTED: ProSAPiP2 protein [Homo sapiens] | 3.74E−03 | 42661180 | 1 | 1 | 1 |
| 426 | DC13 protein [Homo sapiens] | 3.77E−03 | 9910184 | 1 | 1 | 1 |
| 427 | PREDICTED: similar to sperm protein associated wi | 3.79E−03 | 51477491 | 1 | 1 | 1 |
| 428 | UDP-N-acetyl-alpha-D-galactosamine:polypeptide N-acetylgalactosaminylt | 3.79E−03 | 21686971 | 1 | 1 | 1 |
| 429 | Jumonji, AT rich interactive domain 1B (RBP2-like | 3.81E−03 | 19923370 | 2 | 1 | 1 |
| 430 | nitric oxide synthase 1 (neuronal) [Homo sapiens] | 3.81E−03 | 10835173 | 1 | 1 | 1 |
| 431 | golgi phosphoprotein 4; type II Golgi membrane prot | 3.82E−03 | 7657138 | 1 | 1 | 1 |
| 432 | solute carrier family 39 (zinc transporter), member 6; LIV-1 protein, | 3.83E−03 | 12751475 | 1 | 1 | 1 |
| 433 | PREDICTED: similar to ENSANGP00000013733 [Homo sa | 3.85E−03 | 51473057 | 1 | 1 | 1 |
| 434 | vacuolar protein sorting 13A isoform B; chorein; | 3.86E−03 | 15619008 | 1 | 1 | 1 |
| 435 | hypothetical protein FLJ20626 [Homo sapiens] | 3.88E−03 | 8923582 | 1 | 1 | 1 |
| 436 | PREDICTED: similar to bA110H4.2 (similar to membr | 3.97E−03 | 51464932 | 1 | 1 | 1 |
| 437 | PREDICTED: huntingtin interacting protein-1-relat | 3.98E−03 | 29744338 | 1 | 1 | 1 |
| 438 | PREDICTED: similar to KIAA0445 protein [Homo sapi | 4.00E−03 | 51461034 | 1 | 1 | 1 |
| 439 | calcium/calmodulin-dependent protein kinase IV; br | 4.02E−03 | 4502557 | 1 | 1 | 1 |
| 440 | inter-alpha (globulin) inhibitor H5-like; ITI-lik | 4.05E−03 | 38348336 | 1 | 1 | 1 |
| 441 | PREDICTED: similar to serologically defined colon | 4.05E−03 | 41150943 | 1 | 1 | 1 |
| 442 | phosphoinositide-specific phospholipase C beta 1 | 4.07E−03 | 12083581 | 1 | 1 | 1 |
| 443 | vacuolar protein sorting 35; maternal-embryonic 3; vacuolar protein so | 4.10E−03 | 17999541 | 1 | 1 | 1 |
| 444 | M-phase phosphoprotein 1; mitotic kinesin-like protein [Homo sapiens] | 4.11E−03 | 46049114 | 1 | 1 | 1 |
| 445 | lipoyltransferase 1 [Homo sapiens] | 4.13E−03 | 21729878 | 1 | 1 | 1 |
| 446 | sister-of-mammalian grainyhead protein isoform 1; | 4.14E−03 | 38049007 | 1 | 1 | 1 |
| 447 | cadherin 15 preproprotein; M-cadherin; muscle-cadherin; myotubule-cadhe | 4.15E−03 | 4826669 | 1 | 1 | 1 |
| 448 | LOC161577 protein [Homo sapiens] | 4.15E−03 | 38348356 | 1 | 1 | 1 |
| 449 | PREDICTED: hypothetical protein XP_499220 [Homo sa | 4.15E−03 | 51492740 | 1 | 1 | 1 |
| 450 | WW domain binding protein 11; Npw38-binding protei | 4.17E−03 | 7706501 | 1 | 2 | 2 |
| 451 | numb homolog; numb (Drosophila) homolog [Homo sapi | 4.19E−03 | 20070356 | 1 | 1 | 1 |
| 452 | chromosome 10 open reading frame 87; Em:AC061711.1 [Homo sapiens] | 4.19E−03 | 21389373 | 1 | 1 | 1 |
| 453 | PREDICTED: similar to Calpain 9 (Digestive tract- | 4.22E−03 | 51460556 | 1 | 1 | 1 |
| 454 | acetyl-Coenzyme A carboxylase beta [Homo sapiens] | 4.23E−03 | 4501855 | 1 | 1 | 1 |
| 455 | Rho family guanine-nucleotide exchange factor [Ho | 4.32E−03 | 31742505 | 1 | 2 | 2 |
| 456 | hypothetical protein DKFZp547B1713 [Homo sapiens] | 4.35E−03 | 22748815 | 1 | 1 | 1 |
| 457 | secretory leukocyte protease inhibitor precursor; W | 4.40E−03 | 4507065 | 1 | 1 | 1 |
| 458 | hypothetical protein FLJ21477 [Homo sapiens] | 4.46E−03 | 13376749 | 1 | 1 | 1 |
| 459 | E1A binding protein p400; CAGH32 protein; trinucleotide repeat contain | 4.47E−03 | 15805014 | 1 | 1 | 1 |
| 460 | neuroligin 2 [Homo sapiens] | 4.50E−03 | 30840978 | 1 | 1 | 1 |
| 461 | hypothetical protein MGC20781 [Homo sapiens] | 4.52E−03 | 34147472 | 1 | 1 | 1 |
| 462 | FLJ25005 protein [Homo sapiens] | 4.55E−03 | 38570111 | 1 | 4 | 4 |
| 463 | testis expressed sequence 13A [Homo sapiens] | 4.60E−03 | 13775180 | 1 | 1 | 1 |
| 464 | RAC/CDC42 exchange factor isoform 2 [Homo sapiens] | 4.60E−03 | 19311008 | 1 | 1 | 1 |
| 465 | nudix (nucleoside diphosphate linked moiety X)-type motif 11 [Homo sap | 4.64E−03 | 37221177 | 1 | 1 | 1 |
| 466 | heat shock 70 kDa protein 8 isoform 2; heat shock c | 4.64E−03 | 24234686 | 1 | 1 | 1 |
| 467 | PREDICTED: similar to contains transmembrane (TM) | 4.67E−03 | 51474648 | 1 | 1 | 1 |
| 467 | delta globin [Homo sapiens] | 4.67E−03 | 4504351 | 1 | 1 | 1 |
| 468 | DNA methyltransferase 1-associated protein 1 [Hom | 4.68E−03 | 13123776 | 1 | 1 | 1 |
| 469 | coagulation factor XII precursor; Hageman factor [Homo sapiens] | 4.68E−03 | 4503629 | 1 | 1 | 1 |
| 470 | PREDICTED: twist homolog 2 [Homo sapiens] | 4.71E−03 | 51463853 | 1 | 1 | 1 |
| 471 | PREDICTED: similar to RIKEN cDNA G431001E03 gene [Homo sapiens] | 4.73E−03 | 51464570 | 1 | 1 | 1 |
| 472 | cytosolic phosphoenolpyruvate carboxykinase 1; phosphoenolpyruvate car | 4.79E−03 | 32483401 | 1 | 1 | 1 |
| 473 | ubinuclein 1 [Homo sapiens] | 4.83E−03 | 9055374 | 1 | 1 | 1 |
| 474 | macrophage erythroblast attacher; erythroblast macrophage protein [Homo | 4.84E−03 | 5031685 | 1 | 2 | 1 |
| 475 | SH2 domain binding protein 1; TPR-containing, SH2- | 4.93E−03 | 7661950 | 1 | 1 | 1 |
| 476 | interleukin 10 receptor, alpha precursor [Homo sapiens] | 4.94E−03 | 4504633 | 1 | 1 | 1 |
| 477 | PREDICTED: similar to RIKEN cDNA 2210009G21 [Homo sapiens] | 4.97E−03 | 37556081 | 1 | 1 | 1 |
| 478 | protease, serine, 22; protease, serine S1 family member 22; tryptase e | 5.01E−03 | 11545839 | 1 | 1 | 1 |
| 479 | cutaneous T-cell lymphoma tumor antigen se70-2 [H | 5.02E−03 | 31652264 | 1 | 1 | 1 |
| 480 | dihydrofolate reductase [Homo sapiens] | 5.05E−03 | 4503323 | 1 | 1 | 1 |
| 481 | alkylglycerone phosphate synthase precursor [Homo sapiens] | 5.09E−03 | 4501993 | 1 | 1 | 1 |
| 482 | PREDICTED: similar to Golgi autoantigen, golgin subfamily A member 6 ( | 5.11E−03 | 51472431 | 1 | 1 | 1 |
| 483 | palmitoylated membrane protein 7 [Homo sapiens] | 5.12E−03 | 27735101 | 1 | 2 | 2 |
| 484 | hypothetical protein BC008207 [Homo sapiens] | 5.13E−03 | 19923911 | 1 | 1 | 1 |
| 485 | PREDICTED: similar to submaxillary apomucin [Homo | 5.14E−03 | 51471105 | 1 | 2 | 2 |
| 486 | translocase of inner mitochondrial membrane 23 (yeast) homolog; translo | 5.15E−03 | 5454122 | 1 | 1 | 1 |
| 487 | toll-like receptor 9 isoform A precursor [Homo sap | 5.19E−03 | 8394456 | 1 | 1 | 1 |
| 488 | succinate receptor 1; G protein-coupled receptor | 5.22E−03 | 47271392 | 1 | 1 | 1 |
| 489 | PREDICTED: similar to pMesogenin1 [Homo sapiens] | 5.26E−03 | 37539465 | 1 | 1 | 1 |
| 490 | chimerin (chimaerin) 2; Chimerin 2 (GTPase-activating protein, rho, 3); | 5.26E−03 | 4757980 | 1 | 1 | 1 |
| 491 | SRY (sex determining region Y)-box 4; SRY-related HMG-box gene 4; ecotr | 5.28E−03 | 4507163 | 1 | 1 | 1 |
| 492 | PREDICTED: T-box 18 [Homo sapiens] | 5.28E−03 | 51465420 | 1 | 1 | 1 |
| 492 | PREDICTED: hypothetical protein XP_498801 [Homo sa | 5.30E−03 | 51459364 | 1 | 1 | 1 |
| 493 | PREDICTED: follistatin-like 4 [Homo sapiens] | 5.31E−03 | 22049346 | 1 | 1 | 1 |
| 494 | apolipoprotein D precursor [Homo sapiens] | 5.32E−03 | 4502163 | 1 | 1 | 1 |
| 495 | hypothetical protein FLJ40125 [Homo sapiens] | 5.33E−03 | 30425418 | 1 | 1 | 1 |
| 496 | nebulin [Homo sapiens] | 5.35E−03 | 4758794 | 1 | 2 | 2 |
| 497 | testis-specific protein TSP-NY isoform a [Homo sapiens] | 5.38E−03 | 30348972 | 1 | 2 | 2 |
| 498 | MDN1, midasin homolog [Homo sapiens] | 5.39E−03 | 24415404 | 1 | 1 | 1 |
| 499 | hypothetical protein LOC200186 [Homo sapiens] | 5.40E−03 | 32171215 | 1 | 1 | 1 |
| 500 | general transcription factor IIH, polypeptide 1 (62 kD subunit) [Homo sa | 5.41E−03 | 4885365 | 1 | 2 | 1 |
| 501 | T-cell activation kelch repeat protein [Homo sapiens] | 5.41E−03 | 21245132 | 1 | 1 | 1 |
| 502 | KIAA0625 protein [Homo sapiens] | 5.44E−03 | 37620159 | 1 | 1 | 1 |
| 503 | PREDICTED: similar to tigger transposable element | 5.47E−03 | 51467790 | 1 | 1 | 1 |
| 504 | leucine-zipper-like transcriptional regulator, 1; | 5.48E−03 | 47717139 | 1 | 1 | 1 |
| 505 | tumor necrosis factor, alpha-induced protein 1 [Homo sapiens] | 5.50E−03 | 10863937 | 1 | 1 | 1 |
| 506 | hypothetical protein FLJ14768 [Homo sapiens] | 5.53E−03 | 14249548 | 1 | 1 | 1 |
| 507 | myosin light chain 2 [Homo sapiens] | 5.53E−03 | 4557775 | 1 | 1 | 1 |
| 508 | retinitis pigmentosa 1-like 1 [Homo sapiens] | 5.54E−03 | 40255278 | 1 | 1 | 1 |
| 509 | mitochondrial ribosomal protein L32 [Homo sapiens | 5.55E−03 | 13994261 | 1 | 1 | 1 |
| 510 | transaldolase 1; glycerone transferase; dihydroxya | 5.62E−03 | 5803187 | 1 | 1 | 1 |
| 511 | PREDICTED: similar to membrane-spanning proteogly | 5.64E−03 | 51464985 | 1 | 1 | 1 |
| 512 | coiled-coil domain containing 8 [Homo sapiens] | 5.64E−03 | 14042972 | 1 | 1 | 1 |
| 513 | TR4 orphan receptor associated protein TRA16; rep | 5.65E−03 | 28882043 | 1 | 1 | 1 |
| 514 | SLAM family member 7; CD2-like receptor activating cytotoxic cells; 19 | 5.67E−03 | 19923572 | 1 | 1 | 1 |
| 515 | testis nuclear RNA-binding protein [Homo sapiens] | 5.72E−03 | 21245124 | 1 | 1 | 1 |
| 516 | succinate-CoA ligase, ADP-forming, beta subunit; SCS-betaA for ATP spe | 5.79E−03 | 11321583 | 1 | 3 | 1 |
| 517 | transmembrane channel-like 7 [Homo sapiens] | 5.84E−03 | 33636691 | 1 | 1 | 1 |
| 518 | PREDICTED: similar to bA395L14.5 (novel phosphogl | 5.85E−03 | 51460421 | 1 | 1 | 1 |
| 519 | RIC3 protein [Homo sapiens] | 5.90E−03 | 21362040 | 1 | 2 | 2 |
| 520 | zinc finger protein 545 [Homo sapiens] | 5.96E−03 | 28274686 | 1 | 1 | 1 |
| 521 | hypothetical protein FLJ13868 [Homo sapiens] | 5.97E−03 | 12232403 | 1 | 1 | 1 |
| 522 | PREDICTED: similar to Olfactory receptor 52L1 [Homo sapiens] | 5.99E−03 | 51470915 | 1 | 2 | 1 |
| 523 | cadherin 11, type 2 isoform 1 preproprotein; oste | 6.03E−03 | 16306532 | 1 | 1 | 1 |
| 524 | talin 1 [Homo sapiens] | 6.05E−03 | 16753233 | 1 | 1 | 1 |
| 525 | zinc finger protein 625 [Homo sapiens] | 6.05E−03 | 21687161 | 1 | 2 | 1 |
| 526 | general transcription factor IIIC, polypeptide 3, | 6.08E−03 | 6912398 | 1 | 1 | 1 |
| 527 | cystathionine-beta-synthase; serine sulfhydrase; be | 6.11E−03 | 4557415 | 1 | 1 | 1 |
| 528 | nuclear receptor subfamily 4, group A, member 3 is | 6.11E−03 | 27894355 | 1 | 1 | 1 |
| 529 | vitelliform macular dystrophy 2-like 2; bestrophin 4 [Homo sapiens] | 6.11E−03 | 23397576 | 1 | 1 | 1 |
| 530 | hypothetical protein LOC168850 [Homo sapiens] | 6.17E−03 | 39753953 | 1 | 1 | 1 |
| 531 | mutS homolog 5 isoform a; mutS (E. coli) homolog 5 [Homo sapiens] | 6.18E−03 | 26638662 | 1 | 4 | 4 |
| 532 | USH3A protein isoform c; clarin-1 [Homo sapiens] | 6.20E−03 | 16506281 | 1 | 1 | 1 |
| 533 | ATP-binding cassette, sub-family F, member 1; ATP-binding cassette 50; | 6.21E−03 | 10947135 | 1 | 1 | 1 |
| 534 | unc-13 homolog D [Homo sapiens] | 6.22E−03 | 46195765 | 1 | 1 | 1 |
| 535 | nuclear receptor coactivator 7; estrogen receptor associated protein 1 | 6.25E−03 | 42476175 | 1 | 1 | 1 |
| 536 | MYB binding protein 1a; p53-activated protein-2 [Homo sapiens] | 6.25E−03 | 7657351 | 1 | 1 | 1 |
| 537 | elongation factor RNA polymerase II; ELL gene (11- | 6.28E−03 | 5729812 | 1 | 2 | 2 |
| 538 | hypothetical protein MGC10854 [Homo sapiens] | 6.29E−03 | 14150056 | 1 | 1 | 1 |
| 539 | PREDICTED: neuregulin 3 [Homo sapiens] | 6.31E−03 | 51467770 | 1 | 1 | 1 |
| 540 | DEAD (Asp-Glu-Ala-Asp) box polypeptide 18; Myc-re | 6.32E−03 | 38327634 | 1 | 1 | 1 |
| 541 | PREDICTED: similar to centaurin, gamma-like family, member 1; | 6.34E−03 | 51468155 | 1 | 1 | 1 |
| | ARF GTPa | | | | | |
| 542 | KIAA1033 protein [Homo sapiens] | 6.37E−03 | 40018629 | 1 | 1 | 1 |
| 543 | PREDICTED: TBP-interacting protein [Homo sapiens] | 6.46E−03 | 41144277 | 1 | 1 | 1 |
| 543 | PREDICTED: FLJ46675 protein [Homo sapiens] | 6.48E−03 | 51474152 | 1 | 1 | 1 |
| 544 | PREDICTED: similar to bA526D8.2 (novel protein si | 6.51E−03 | 51467642 | 1 | 1 | 1 |
| 544 | Rho-specific guanine nucleotide exchange factor p | 6.68E−03 | 41327769 | 1 | 1 | 1 |
| 545 | polypeptide N-acetylgalactosaminyltransferase 6; Ga | 6.69E−03 | 6005766 | 1 | 1 | 1 |
| 546 | interleukin 13 receptor, alpha 1 precursor; IL13 receptor alpha-1 chain | 6.69E−03 | 4504647 | 1 | 1 | 1 |
| 547 | PREDICTED: SHC (Src homology 2 domain containing) | 6.77E−03 | 51474945 | 1 | 1 | 1 |
| 548 | PREDICTED: KIAA1205 [Homo sapiens] | 6.79E−03 | 51474761 | 1 | 1 | 1 |
| 549 | UNC13 (C. elegans)-like; homolog of rat Munc13 (d | 6.80E−03 | 38176154 | 1 | 1 | 1 |
| 550 | AT-hook transcription factor; AT-hook transcripti | 6.81E−03 | 37620171 | 1 | 1 | 1 |
| 551 | TBP-associated factor 9L; neuronal cell death-rel | 6.83E−03 | 20070280 | 1 | 1 | 1 |
| 552 | thrombospondin 2 precursor [Homo sapiens] | 6.85E−03 | 40317628 | 1 | 1 | 1 |
| 553 | hypothetical protein BC011981 [Homo sapiens] | 6.90E−03 | 19923935 | 1 | 1 | 1 |
| 554 | breast cancer 2, early onset; Fanconi anemia, comp | 6.95E−03 | 4502451 | 1 | 1 | 1 |
| 555 | acyl-Coenzyme A oxidase 3, pristanoyl [Homo sapiens | 6.97E−03 | 4501871 | 1 | 1 | 1 |
| 556 | A-kinase anchor protein 9 isoform 2; A-kinase anc | 7.07E−03 | 22538387 | 1 | 1 | 1 |
| 557 | PREDICTED: similar to elongation factor Tu GTP bin | 7.13E−03 | 51472670 | 1 | 1 | 1 |
| 558 | TUDOR gene similar [Homo sapiens] | 7.14E−03 | 32996737 | 1 | 1 | 1 |
| 559 | mitogen-activated protein kinase 10 isoform 3; MAP kinase; JNK3 alpha | 7.21E−03 | 20986506 | 1 | 1 | 1 |
| 560 | bifunctional phosphopantetheine adenylyl transferase/dephospho CoA kin | 7.22E−03 | 46048207 | 1 | 1 | 1 |
| 561 | PREDICTED: similar to Fc receptor homolog expressed in B cells; Fc rec | 7.24E−03 | 51458812 | 1 | 1 | 1 |
| 562 | FLJ46072 protein [Homo sapiens] | 7.24E−03 | 38348290 | 1 | 1 | 1 |
| 563 | PREDICTED: hypothetical protein DKFZp762K222 [Homo sapiens] | 7.47E−03 | 51464327 | 1 | 1 | 1 |
| 564 | PREDICTED: similar to eukaryotic translation elo | 7.50E−03 | 51474994 | 1 | 1 | 1 |
| 565 | hypothetical protein FLJ10706 [Homo sapiens] | 7.51E−03 | 40254931 | 1 | 1 | 1 |
| 566 | hypothetical protein MGC4308 [Homo sapiens] | 7.51E−03 | 14150167 | 1 | 2 | 2 |
| 567 | KIAA1632 protein [Homo sapiens] | 7.53E−03 | 46195733 | 1 | 1 | 1 |
| 567 | PREDICTED: PTPRF interacting protein, binding pro | 7.54E−03 | 20561108 | 1 | 1 | 1 |
| 568 | PREDICTED: similar to osteotesticular protein tyr | 7.58E−03 | 51458868 | 1 | 1 | 1 |
| 569 | hypothetical protein CG003 [Homo sapiens] | 7.59E−03 | 12957488 | 1 | 1 | 1 |
| 570 | cell cycle progression 1; cell cycle progression | 7.64E−03 | 28395035 | 1 | 1 | 1 |
| 571 | mbt domain containing 1 [Homo sapiens] | 7.70E−03 | 8923059 | 1 | 1 | 1 |
| 572 | proline arginine-rich end leucine-rich repeat prot | 7.89E−03 | 41349454 | 1 | 1 | 1 |
| 573 | protein tyrosine phosphatase, non-receptor type 3 | 7.93E−03 | 18104986 | 1 | 1 | 1 |
| 574 | PREDICTED: similar to KIAA2033 protein [Homo sapi | 7.94E−03 | 51464267 | 1 | 1 | 1 |
| 575 | hypothetical protein FLJ21019 [Homo sapiens] | 7.94E−03 | 40255047 | 1 | 1 | 1 |
| 576 | PREDICTED: myosin VI [Homo sapiens] | 7.97E−03 | 42657517 | 1 | 1 | 1 |
| 577 | PREDICTED: similar to Ig heavy chain V-I region HG3 precursor [Homo sa | 7.98E−03 | 51472657 | 1 | 1 | 1 |
| 578 | FLJ45248 protein [Homo sapiens] | 7.98E−03 | 46409638 | 1 | 1 | 1 |
| 579 | gelsolin isoform a [Homo sapiens] | 7.98E−03 | 4504165 | 1 | 1 | 1 |
| 580 | oxysterol-binding protein-like 1A isoform B; oxys | 8.02E−03 | 19718741 | 1 | 1 | 1 |
| 581 | rearranged L-myc fusion sequence; Zn-15 related [Homo sapiens] | 8.08E−03 | 6912632 | 1 | 1 | 1 |
| 582 | cardiomyopathy associated 5; 2310076E16Rik [Homo sapiens] | 8.25E−03 | 32698780 | 1 | 1 | 1 |
| 583 | ADP-ribosylation factor guanine nucleotide-exchange factor 2; brefeldin | 8.25E−03 | 5453573 | 1 | 1 | 1 |
| 584 | sidekick 2; Drosophila sidekick-like; chicken sid | 8.27E−03 | 21735577 | 1 | 1 | 1 |
| 585 | similar to golgi autoantigen, golgin subfamily a, | 8.29E−03 | 38044110 | 1 | 1 | 1 |
| 586 | CTP: phosphocholine cytidylyltransferase b [Homo s | 8.34E−03 | 21361202 | 1 | 1 | 1 |
| 587 | zinc finger protein 295 [Homo sapiens] | 8.35E−03 | 40254945 | 1 | 1 | 1 |
| 588 | adenylate kinase 3 alpha like; adenylate kinase 6 | 8.35E−03 | 19923437 | 1 | 1 | 1 |
| 589 | ataxia telangiectasia mutated protein isoform 1; | 8.37E−03 | 20336203 | 1 | 1 | 1 |
| 590 | ring finger protein 31 [Homo sapiens] | 8.40E−03 | 38045940 | 1 | 1 | 1 |
| 591 | hypothetical protein DKFZp564J047 [Homo sapiens] | 8.44E−03 | 14149781 | 1 | 1 | 1 |
| 592 | VGF nerve growth factor inducible precursor; neuro | 8.47E−03 | 17136078 | 1 | 1 | 1 |
| 593 | LIM domain kinase 2 isoform 2a [Homo sapiens] | 8.54E−03 | 5031869 | 1 | 1 | 1 |
| 594 | spermiogenesis associated serine/threonine kinase | 8.60E−03 | 14042947 | 1 | 1 | 1 |
| 595 | fatty acid binding protein 5 (psoriasis-associated) | 8.62E−03 | 4557581 | 1 | 1 | 1 |
| 596 | PREDICTED: KIAA1549 protein [Homo sapiens] | 8.65E−03 | 41199251 | 1 | 1 | 1 |
| 597 | meningioma expressed antigen 5 (hyaluronidase); hy | 8.71E−03 | 11024698 | 1 | 1 | 1 |
| 598 | PREDICTED: similar to apopolysialoglycoprotein - | 8.72E−03 | 51464378 | 1 | 1 | 1 |
| 599 | zinc finger protein 135 (clone pHZ-17); zinc finger protein 61 [Homo s | 8.75E−03 | 34419633 | 1 | 1 | 1 |
| 600 | kelch-like 17; actinfilin [Homo sapiens] | 8.82E−03 | 38194229 | 1 | 1 | 1 |
| 601 | mitochondrial ribosomal protein S31; imogen 38 [Ho | 8.84E−03 | 5031787 | 1 | 1 | 1 |
| 601 | coatomer protein complex, subunit beta 2 (beta pri | 8.85E−03 | 4758032 | 1 | 1 | 1 |
| 602 | PREDICTED: KIAA0767 protein [Homo sapiens] | 8.86E−03 | 17485400 | 1 | 1 | 1 |
| 603 | synaptosomal-associated protein 25 isoform SNAP25A | 8.87E−03 | 18765733 | 1 | 1 | 1 |
| 604 | chromosome 14 open reading frame 8; p25 beta [Homo sapiens] | 8.89E−03 | 27777659 | 1 | 1 | 1 |
| 605 | ubiquitin specific protease 28 [Homo sapiens] | 8.91E−03 | 16507200 | 1 | 1 | 1 |
| 606 | zinc finger protein 553 [Homo sapiens] | 8.94E−03 | 22749325 | 1 | 1 | 1 |
| 607 | membrane associated guanylate kinase interacting protein-like 1 [Homo | 9.00E−03 | 27735139 | 1 | 1 | 1 |
| 608 | PREDICTED: hypothetical protein FLJ11811 [Homo sa | 9.02E−03 | 42660246 | 1 | 1 | 1 |
| 609 | protein kinase C substrate 80K-H; glucosidase II, | 9.04E−03 | 4506077 | 1 | 1 | 1 |
| 610 | polymerase (DNA directed), delta 2, regulatory subunit; polymerase (DNA | 9.06E−03 | 5453924 | 1 | 1 | 1 |
| 611 | PREDICTED: hypothetical protein XP_499090 [Homo s | 9.07E−03 | 51465873 | 1 | 1 | 1 |
| 612 | peroxiredoxin 3 isoform b; antioxidant protein 1; thioredoxin-dependen | 9.08E−03 | 32483377 | 1 | 1 | 1 |
| 613 | similar to hypothetical protein FLJ13659 [Homo sa | 9.12E−03 | 21687266 | 1 | 1 | 1 |
| 614 | PREDICTED: KIAA0542 gene product [Homo sapiens] | 9.15E−03 | 51476003 | 1 | 1 | 1 |
| 615 | PREDICTED: 5-azacytidine induced 1 [Homo sapiens] | 9.17E−03 | 51474428 | 1 | 1 | 1 |
| 616 | thyrotrophic embryonic factor; thyrotroph embryonic factor [Homo sapien | 9.22E−03 | 4507431 | 1 | 1 | 1 |
| 617 | zinc finger, CW-type with coiled-coil domain 3; n | 9.24E−03 | 28872812 | 1 | 1 | 1 |
| 618 | betaine-homocysteine methyltransferase 2 [Homo sa | 9.24E−03 | 13162290 | 1 | 1 | 1 |
| 619 | FLJ45949 protein [Homo sapiens] | 9.25E−03 | 46409412 | 1 | 1 | 1 |
| 620 | general transcription factor IIIC, polypeptide 1, alpha 220 kDa; general | 9.27E−03 | 4753161 | 1 | 1 | 1 |
| 621 | PR domain containing 5 [Homo sapiens] | 9.30E−03 | 41349476 | 1 | 1 | 1 |
| 622 | glycosyltransferase-like 1B; ortholog of mouse glycosyltransferase-lik | 9.33E−03 | 33285008 | 1 | 1 | 1 |
| 623 | bullous pemphigoid antigen 1 isoform 1; bullous p | 9.37E−03 | 34577047 | 1 | 3 | 3 |
| 624 | mitofusin 1 isoform 1; putative transmembrane GTPase [Homo sapiens] | 9.39E−03 | 45269137 | 1 | 1 | 1 |
| 624 | hypothetical protein FLJ37440 [Homo sapiens] | 9.41E−03 | 23397471 | 1 | 1 | 1 |
| 625 | polycystic kidney disease and receptor for egg jel | 9.42E−03 | 5174633 | 1 | 1 | 1 |
| 626 | doublecortin and CaM kinase-like 1; doublecortin-I | 9.42E−03 | 4758128 | 1 | 1 | 1 |
| 627 | PREDICTED: similar to synaptotagmin-like protein | 9.42E−03 | 18562480 | 1 | 1 | 1 |
| 628 | latrophilin 3; latrophilin homolog 3 (cow); lectomedin 3 [Homo sapiens | 9.52E−03 | 14149677 | 1 | 1 | 1 |
| 629 | down-regulated in adenoma protein; down-regulated | 9.52E−03 | 4557535 | 1 | 1 | 1 |
| 630 | integrin-linked kinase-associated protein phosphat | 9.52E−03 | 13540531 | 1 | 1 | 1 |
| 631 | S-adenosylhomocysteine hydrolase; adenosylhomocyst | 9.58E−03 | 9951915 | 1 | 1 | 1 |
| 632 | A-kinase anchor protein 8; A-kinase anchor protein, 95 kDa [Homo sapiens | 9.60E−03 | 5031579 | 1 | 1 | 1 |
| 633 | PREDICTED: FYVE, RhoGEF and PH domain containing 5 [Homo sapiens] | 9.64E−03 | 51463913 | 1 | 1 | 1 |
| 634 | PREDICTED: similar to KIAA1074 protein [Homo sapiens] | 9.67E−03 | 42661355 | 1 | 1 | 1 |
| 635 | ryanodine receptor 2 [Homo sapiens] | 9.67E−03 | 4506757 | 1 | 1 | 1 |
| 636 | actin related protein M1 [Homo sapiens] | 9.72E−03 | 19549325 | 1 | 1 | 1 |
| 637 | chromosome 19 open reading frame 21 [Homo sapiens | 9.72E−03 | 27735067 | 1 | 1 | 1 |
| 638 | mirror-image polydactyly 1; mirror-image polydact | 9.72E−03 | 23503325 | 1 | 1 | 1 |
| 639 | early growth response 1; nerve growth factor-induc | 9.73E−03 | 4503493 | 1 | 1 | 1 |
| 640 | protease, serine, 15; Lon protease-like protein; | 9.76E−03 | 21396489 | 1 | 1 | 1 |
| 641 | centromere protein F (350/400 kD); centromere prot | 9.77E−03 | 14670381 | 1 | 1 | 1 |
| 642 | ring finger and WD repeat domain 1 isoform 1; rin | 9.78E−03 | 45594312 | 1 | 1 | 1 |
| 643 | PREDICTED: KIAA0226 gene product [Homo sapiens] | 9.79E−03 | 51464047 | 1 | 1 | 1 |
| 644 | phospholipase C, gamma 2 (phosphatidylinositol-spe | 9.89E−03 | 4505871 | 1 | 1 | 1 |
| 645 | transforming, acidic coiled-coil containing prote | 9.91E−03 | 11119414 | 1 | 1 | 1 |
| 646 | cadherin 10, type 2 preproprotein; cadherin-10; T2-cadherin [Homo sapi | 9.94E−03 | 16306530 | 1 | 1 | 1 |
| 647 | insulysin; insulinase [Homo sapiens] | 1.00E−02 | 4826770 | 1 | 1 | 1 |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |
| | Protein Name | P (probability) | Accession No | Id Pep | Id No on 19 | Pat No |

TABLE 3.3
Proteins identified in vitreous fluid (III)

| | | | | Probability of |
|---|---|---|---|---|
| | | | Peptide | randomized |
| | Protein | Accession number | Hits | identification |
| 1 | PREDICTED: KIAA0542 gene product [Homo sapiens] | 51476003 | 1 | 9.15E−03 |
| 2 | PREDICTED: hypothetical protein XP_498867 [Homo sapiens] | 51475312 | 1 | 7.61E−03 |
| 3 | PREDICTED: KIAA0179 [Homo sapiens] | 51475296 | 1 | 3.71E−03 |
| 4 | PREDICTED: similar to 60S ribosomal protein L23a [Homo sapiens] | 51474226 | 1 | 6.71E−04 |
| 5 | PREDICTED: similar to Ig heavy chain V-I region HG3 precursor [Homo sa | 51472657 | 1 | 7.98E−03 |
| 6 | PREDICTED: hypothetical protein FLJ25756 [Homo sapiens] | 51472397 | 1 | 7.01E−03 |
| 7 | PREDICTED: GCN1 general control of amino-acid synthesis 1-like 1 [Homo | 51471344 | 1 | 2.23E−03 |
| 8 | PREDICTED: similar to Olfactory receptor 52L1 [Homo sapiens] | 51470915 | 1 | 9.64E−03 |
| 9 | PREDICTED: KIAA0367 protein [Homo sapiens] | 51467193 | 1 | 1.19E−03 |
| 10 | PREDICTED: T-box 18 [Homo sapiens] | 51465420 | 1 | 5.28E−03 |
| 11 | PREDICTED: similar to RIKEN cDNA G431001E03 gene [Homo sapiens] | 51464570 | 1 | 4.73E−03 |
| 12 | PREDICTED: hypothetical protein DKFZp762K222 [Homo sapiens] | 51464327 | 1 | 7.47E−03 |
| 13 | PREDICTED: KIAA0226 gene product [Homo sapiens] | 51464047 | 1 | 9.79E−03 |
| 14 | PREDICTED: FYVE, RhoGEF and PH domain containing 5 [Homo sapiens] | 51463913 | 1 | 9.64E−03 |
| 15 | PREDICTED: similar to sin3 associated polypeptide p18 [Homo sapiens] | 51461003 | 1 | 1.83E−03 |
| 16 | PREDICTED: cardiomyopathy associated 3 [Homo sapiens] | 51460893 | 1 | 6.18E−04 |
| 17 | PREDICTED: KIAA1414 protein [Homo sapiens] | 51460558 | 1 | 4.99E−03 |
| 18 | PREDICTED: similar to 40S ribosomal protein S7 (S8) [Homo sapiens] | 51459063 | 1 | 2.47E−03 |
| 19 | PREDICTED: similar to Fc receptor homolog expressed in B cells; Fc rec | 51458812 | 1 | 7.24E−03 |
| 20 | hypothetical protein LOC255189 [Homo sapiens] | 47106065 | 1 | 3.82E−03 |
| 21 | zinc finger, MYND domain containing 11 isoform b; adenovirus 5 E1A bin | 47078243 | 1 | 2.97E−03 |
| 22 | haptoglobin-related protein; Haptoglobin-related locus [Homo sapiens] | 45580723 | 2 | 6.97E−05 |
| 23 | MAS-related GPR, member D; mas-related G protein-coupled MRGD [Homo sa | 42794265 | 1 | 2.48E−03 |
| 24 | KIAA0317 [Homo sapiens] | 42734315 | 1 | 1.98E−03 |
| 25 | clusterin isoform 1; complement-associated protein SP-40 [Homo sapiens | 42716297 | 1 | 5.04E−12 |
| 26 | PREDICTED: similar to KIAA1074 protein [Homo sapiens] | 42661355 | 1 | 9.67E−03 |
| 27 | PREDICTED: KIAA1030 protein [Homo sapiens] | 42659643 | 1 | 6.08E−04 |
| 28 | PREDICTED: similar to tudor domain containing 6 protein [Homo sapiens] | 42657656 | 1 | 1.40E−03 |
| 29 | PREDICTED: hypothetical protein XP_376099 [Homo sapiens] | 42656388 | 1 | 6.72E−05 |
| 30 | vacuolar protein sorting 13C protein [Homo sapiens] | 42544121 | 1 | 3.48E−03 |
| 31 | semaphorin 3D; collapsin 2 [Homo sapiens] | 41406086 | 1 | 9.26E−03 |
| 32 | PREDICTED: hypothetical protein XP_373740 [Homo sapiens] | 41222847 | 1 | 5.96E−05 |
| 33 | PREDICTED: TBP-interacting protein [Homo sapiens] | 41144277 | 1 | 6.46E−03 |
| 34 | thrombospondin 2 precursor [Homo sapiens] | 40317628 | 1 | 6.85E−03 |
| 35 | hypothetical protein MGC34732 [Homo sapiens] | 40255180 | 1 | 9.92E−05 |
| 36 | zinc finger protein 295 [Homo sapiens] | 40254945 | 1 | 8.35E−03 |
| 37 | serine (or cysteine) proteinase inhibitor, clade F (alpha-2 antiplasmi | 39725934 | 2 | 3.34E−08 |
| 38 | kelch-like 17; actinfilin [Homo sapiens] | 38194229 | 1 | 8.82E−03 |
| 39 | gelsolin isoform b [Homo sapiens] | 38044288 | 2 | 2.15E−08 |
| 40 | KIAA0625 protein [Homo sapiens] | 37620159 | 1 | 5.44E−03 |
| 41 | PREDICTED: similar to RIKEN cDNA 2210009G21 [Homo sapiens] | 37556081 | 1 | 4.97E−03 |
| 42 | nudix (nucleoside diphosphate linked moiety X)-type motif 11 [Homo sap | 37221177 | 1 | 4.64E−03 |
| 43 | hypothetical protein DKFZp434O0527 [Homo sapiens] | 34916026 | 1 | 7.73E−03 |
| 44 | kelch-like 8 [Homo sapiens] | 34101268 | 1 | 2.43E−03 |
| 45 | biliverdin reductase A [Homo sapiens] | 33589854 | 1 | 1.86E−03 |
| 46 | bromodomain adjacent to zinc finger domain, 1A isoform a; ATP-dependen | 32967603 | 1 | 1.48E−03 |
| 47 | vitamin D-binding protein precursor; vitamin D-binding alpha-globulin | 32483410 | 6 | 1.40E−12 |
| 48 | peroxiredoxin 3 isoform b; antioxidant protein 1; thioredoxin-dependen | 32483377 | 1 | 9.08E−03 |
| 49 | Ral GEF with PH domain and SH3 binding motif 2; Ral-A exchange factor | 32441283 | 1 | 3.25E−03 |
| 50 | prostaglandin D2 synthase 21 kDa; prostaglandin D synthase; prostagland | 32171249 | 1 | 1.22E−09 |
| 51 | hypothetical protein MGC5601; similar to acyl-CoA dehydrogenase [Homo | 31543201 | 1 | 5.87E−04 |
| 52 | zinc finger protein 606 [Homo sapiens] | 31377839 | 1 | 9.52E−04 |
| 53 | family with sequence similarity 29, member A [Homo sapiens] | 31377562 | 1 | 1.71E−04 |
| 54 | phosphatidylinositol-4-phosphate 5-kinase, type I, gamma; phosphatidyl | 31317309 | 1 | 3.65E−03 |
| 55 | testis-specific protein TSP-NY isoform a [Homo sapiens] | 30348972 | 1 | 5.38E−03 |
| 56 | H63 breast cancer expressed gene isoform a; gene associated with HER-2 | 29826289 | 1 | 1.59E−03 |
| 57 | plexin B3; plexin-B3; plexin 6 [Homo sapiens] | 29336063 | 1 | 9.96E−03 |
| 58 | eukaryotic translation initiation factor 2C, 3 isoform a; argonaute 3 | 29294647 | 1 | 4.80E−03 |
| 59 | dual oxidase 2 precursor; dual oxidase-like domains 2; nicotinamide ad | 28872753 | 1 | 2.36E−04 |
| 60 | KIAA1404 protein [Homo sapiens] | 28626521 | 1 | 3.58E−03 |
| 61 | Bardet-Biedl syndrome 1 [Homo sapiens] | 28395045 | 1 | 2.36E−03 |
| 62 | zinc finger protein 545 [Homo sapiens] | 28274686 | 1 | 5.96E−03 |
| 63 | chromosome 14 open reading frame 8; p25 beta [Homo sapiens] | 27777659 | 1 | 8.89E−03 |
| 64 | membrane associated guanylate kinase interacting protein-like 1 [Homo | 27735139 | 1 | 9.00E−03 |
| 65 | hypothetical protein FLJ32440 [Homo sapiens] | 27734761 | 1 | 3.97E−05 |
| 66 | sperm associated antigen 6 isoform 2; sperm flagellar protein; axoneme | 27262641 | 1 | 2.37E−04 |
| 67 | mutS homolog 5 isoform a; mutS (E. coli) homolog 5 [Homo sapiens] | 26638662 | 1 | 6.18E−03 |
| 68 | transcription factor-like protein 4 isoform gamma; MAX-like bHLHZIP pr | 24586667 | 1 | 2.12E−03 |
| 69 | myosin IIIA; deafness, autosomal recessive 30 [Homo sapiens] | 24586657 | 1 | 9.51E−04 |
| 70 | MDN1, midasin homolog [Homo sapiens] | 24415404 | 1 | 5.39E−03 |
| 71 | vitelliform macular dystrophy 2-like 2; bestrophin 4 [Homo sapiens] | 23397576 | 1 | 6.11E−03 |
| 72 | elongation of very long chain fatty acids like 3 [Homo sapiens] | 23097310 | 1 | 1.06E−03 |
| 73 | zinc finger protein 560 [Homo sapiens] | 22749003 | 1 | 2.18E−03 |
| 74 | junctophilin 1; mitsugumin72; junctophilin type1 [Homo sapiens] | 21735575 | 1 | 2.43E−03 |
| 75 | UDP-N-acetyl-alpha-D-galactosamine:polypeptide N-acetylgalactosaminylt | 21686971 | 1 | 3.79E−03 |
| 76 | hypothetical protein MGC27016 [Homo sapiens] | 21450675 | 1 | 1.48E−03 |
| 77 | DnaJ (Hsp40) homolog, subfamily C, member 1 [Homo sapiens] | 21361912 | 1 | 3.37E−03 |
| 78 | peptidoglycan recognition protein L precursor [Homo sapiens] | 21361845 | 1 | 4.73E−04 |
| 79 | Tax1 (human T-cell leukemia virus type I) binding protein 1; tax1-bind | 21361682 | 1 | 1.14E−03 |
| 80 | serine (or cysteine) proteinase inhibitor, clade A (alpha-1 antiprotei | 21361198 | 12 | 4.44E−14 |
| 81 | T-cell activation kelch repeat protein [Homo sapiens] | 21245132 | 1 | 5.41E−03 |
| 82 | testis nuclear RNA-binding protein [Homo sapiens] | 21245124 | 1 | 5.72E−03 |
| 83 | alpha 1B-glycoprotein [Homo sapiens] | 21071030 | 2 | 1.13E−10 |
| 84 | mitogen-activated protein kinase 10 isoform 3; MAP kinase; JNK3 alpha | 20986506 | 1 | 7.21E−03 |
| 85 | toll-like receptor 8 isoform 1 [Homo sapiens] | 20302166 | 1 | 2.90E−03 |
| 86 | SLAM family member 7; CD2-like receptor activating cytotoxic cells; 19 | 19923572 | 1 | 5.67E−03 |
| 87 | RAC/CDC42 exchange factor isoform 2 [Homo sapiens] | 19311008 | 1 | 4.60E−03 |
| 88 | vitronectin precursor; serum spreading factor; somatomedin B; compleme | 18201911 | 1 | 1.38E−03 |
| 89 | ribosomal protein L10-like protein [Homo sapiens] | 18152783 | 1 | 1.50E−03 |
| 90 | vacuolar protein sorting 35; maternal-embryonic 3; vacuolar protein so | 17999541 | 1 | 4.10E−03 |
| 91 | talin 1 [Homo sapiens] | 16753233 | 1 | 6.05E−03 |
| 92 | ankyrin repeat domain 30A; breast cancer antigen NY-BR-1 [Homo sapiens | 16506285 | 1 | 3.16E−03 |
| 93 | cadherin 10, type 2 preproprotein; cadherin-10; T2-cadherin [Homo sapi | 16306530 | 1 | 9.94E−03 |
| 94 | arginyl-tRNA synthetase [Homo sapiens] | 15149476 | 1 | 4.28E−03 |
| 95 | complement component 2 precursor; C3/C5 convertase [Homo sapiens] | 14550407 | 1 | 8.23E−04 |
| 96 | hypothetical protein MGC4308 [Homo sapiens] | 14150167 | 1 | 7.51E−03 |
| 97 | latrophilin 3; latrophilin homolog 3 (cow); lectomedin 3 [Homo sapiens | 14149677 | 1 | 9.52E−03 |
| 98 | ubiquitin specific protease 15; deubiquitinating enzyme [Homo sapiens] | 14149627 | 1 | 6.90E−04 |
| 99 | pantothenate kinase 3; pantothenic acid kinase [Homo sapiens] | 13375789 | 1 | 1.01E−03 |
| 100 | solute carrier family 39 (zinc transporter), member 6; LIV-1 protein, | 12751475 | 1 | 3.83E−03 |
| 101 | dual specificity phosphatase 5; serine/threonine specific protein phos | 12707566 | 1 | 1.07E−03 |
| 102 | protease, serine, 22; protease, serine S1 family member 22; tryptase e | 11545839 | 1 | 5.01E−03 |
| 103 | supervillin isoform 2; membrane-associated F-actin binding protein p20 | 11496982 | 1 | 1.75E−03 |
| 104 | alpha-2-plasmin inhibitor; alpha-2-antiplasmin [Homo sapiens] | 11386143 | 1 | 3.57E−06 |
| 105 | hemopexin [Homo sapiens] | 11321561 | 9 | 1.55E−10 |
| 106 | tuberous sclerosis 2 isoform 2; tuberin isoform 1; tuberin isoform 2; | 10938008 | 1 | 2.96E−04 |
| 107 | tumor necrosis factor, alpha-induced protein 1 [Homo sapiens] | 10863937 | 1 | 5.50E−03 |
| 108 | orosomucoid 1 precursor; alpha-1-acid glycoprotein 1; Orosomucoid-1 (al | 9257232 | 6 | 6.94E−10 |
| 109 | hypothetical protein FLJ20580 [Homo sapiens] | 8923541 | 1 | 2.18E−03 |
| 110 | ankyrin repeat and SOCS box-containing 6 isoform 1 [Homo sapiens] | 8923516 | 1 | 2.02E−03 |
| 111 | hypothetical protein DKFZp434H2215 [Homo sapiens] | 8922138 | 1 | 1.96E−03 |
| 112 | hepatoma-derived growth factor, related protein 3; hepatoma-derived gro | 7705320 | 1 | 3.24E−03 |
| 113 | GTPase regulator associated with the focal adhesion kinase pp125; GTPas | 7662208 | 1 | 1.75E−03 |
| 114 | opticin; opticin, oculoglycan; oculoglycan [Homo sapiens] | 7657419 | 1 | 1.57E−03 |
| 115 | MYB binding protein 1a; p53-activated protein-2 [Homo sapiens] | 7657351 | 1 | 6.25E−03 |
| 116 | T-box 21; T-box expressed in T cells; T-cell-specific T-box transcripti | 7019549 | 1 | 2.05E−03 |
| 117 | rearranged L-myc fusion sequence; Zn-15 related [Homo sapiens] | 6912632 | 1 | 8.08E−03 |
| 118 | RBP4 gene product [Homo sapiens] | 5803139 | 3 | 1.46E−12 |
| 119 | polymerase (DNA directed), delta 2, regulatory subunit; polymerase (DNA | 5453924 | 1 | 9.06E−03 |
| 120 | ADP-ribosylation factor guanine nucleotide-exchange factor 2; brefeldin | 5453573 | 1 | 8.25E−03 |
| 121 | insulin receptor substrate 1 [Homo sapiens] | 5031805 | 1 | 3.11E−03 |
| 122 | macrophage erythroblast attacher; erythroblast macrophage protein [Homo | 5031685 | 1 | 4.84E−03 |
| 123 | A-kinase anchor protein 8; A-kinase anchor protein, 95 kDa [Homo sapiens | 5031579 | 1 | 9.60E−03 |
| 124 | general transcription factor IIH, polypeptide 1 (62 kD subunit) [Homo sa | 4885365 | 1 | 5.41E−03 |
| 125 | haptoglobin [Homo sapiens] | 4826762 | 3 | 8.18E−07 |
| 126 | fragile X mental retardation-related protein 1; Fragile X mental retard | 4826736 | 1 | 3.60E−03 |
| 127 | cadherin 15 preproprotein; M-cadherin; muscle-cadherin; myotubule-cadhe | 4826669 | 1 | 4.15E−03 |
| 128 | nebulin [Homo sapiens] | 4758794 | 1 | 5.35E−03 |
| 129 | growth factor receptor-bound protein 14 [Homo sapiens] | 4758478 | 1 | 3.32E−03 |
| 130 | golgi autoantigen, golgin subfamily b, macrogolgin (with transmembrane | 4758454 | 1 | 5.05E−04 |
| 131 | dual specificity phosphatase 8; serine/threonine specific protein phosp | 4758212 | 1 | 1.48E−04 |
| 132 | chimerin (chimaerin) 2; Chimerin 2 (GTPase-activating protein, rho, 3); | 4757980 | 1 | 5.26E−03 |
| 133 | beta-2-microglobulin precursor [Homo sapiens] | 4757826 | 2 | 2.02E−11 |
| 134 | general transcription factor IIIC, polypeptide 1, alpha 220 kDa; general | 4753161 | 1 | 9.27E−03 |
| 135 | phosphodiesterase 6A, alpha subunit [Homo sapiens] | 4585864 | 1 | 8.81E−03 |
| 136 | transferrin; PRO2086 protein [Homo sapiens] | 4557871 | 34 | 1.33E−14 |
| 137 | potassium voltage-gated channel, shaker-related subfamily, member 1 [Ho | 4557685 | 1 | 2.42E−03 |
| 138 | ceruloplasmin (ferroxidase); Ceruloplasmin [Homo sapiens] | 4557485 | 4 | 4.83E−06 |
| 139 | complement component 3 precursor; acylation-stimulating protein cleavag | 4557385 | 7 | 5.04E−09 |
| 140 | complement component 1 inhibitor precursor [Homo sapiens] | 4557379 | 1 | 1.35E−06 |
| 141 | beta-2-glycoprotein I precursor [Homo sapiens] | 4557327 | 4 | 2.14E−10 |
| 142 | apolipoprotein A-I precursor [Homo sapiens] | 4557321 | 10 | 8.46E−09 |
| 143 | angiotensinogen precursor; angiotensin II precursor pre-angiotensinoge | 4557287 | 1 | 3.81E−05 |
| 144 | skeletal muscle specific actinin, alpha 3 [Homo sapiens] | 4557241 | 1 | 3.34E−04 |
| 145 | transthyretin; prealbumin [Homo sapiens] | 4507725 | 7 | 2.22E−14 |
| 146 | thyrotrophic embryonic factor; thyrotroph embryonic factor [Homo sapien | 4507431 | 1 | 9.22E−03 |
| 147 | ryanodine receptor 2 [Homo sapiens] | 4506757 | 1 | 9.67E−03 |
| 148 | pregnancy-zone protein; Pregnancy zone protein [Homo sapiens] | 4506355 | 1 | 2.56E−06 |
| 149 | plasminogen [Homo sapiens] | 4505881 | 1 | 6.54E−04 |
| 150 | orosomucoid 2; alpha-1-acid glycoprotein, type 2 [Homo sapiens] | 4505529 | 2 | 2.73E−13 |
| 151 | neogenin homolog 1; neogenin (chicken) homolog 1 [Homo sapiens] | 4505375 | 1 | 6.20E−03 |
| 152 | kininogen 1; alpha-2-thiol proteinase inhibitor; bradykinin [Homo sapie | 4504893 | 3 | 1.66E−05 |
| 153 | interleukin 13 receptor alpha 1 precursor; IL13 receptor alpha-1 chain | 4504647 | 1 | 6.69E−03 |
| 154 | interleukin 10 receptor, alpha precursor [Homo sapiens] | 4504633 | 1 | 4.94E−03 |
| 155 | I factor (complement) [Homo sapiens] | 4504579 | 1 | 9.49E−05 |
| 156 | histidine-rich glycoprotein precursor; histidine-proline rich glycoprot | 4504489 | 1 | 2.37E−07 |
| 157 | coagulation factor XII precursor; Hageman factor [Homo sapiens] | 4503629 | 1 | 4.68E−03 |
| 158 | dihydrofolate reductase [Homo sapiens] | 4503323 | 1 | 5.05E−03 |
| 159 | centromere protein C 1; Centromere autoantigen C1 [Homo sapiens] | 4502779 | 1 | 1.98E−03 |
| 160 | corticosteroid binding globulin precursor; corticosteroid binding globu | 4502595 | 1 | 1.35E−03 |
| 161 | complement component 9 [Homo sapiens] | 4502511 | 1 | 9.42E−03 |
| 162 | complement component 4B proprotein [Homo sapiens] | 4502501 | 3 | 3.11E−14 |
| 163 | complement factor B preproprotein; C3 proactivator; C3 proaccelerator; | 4502397 | 4 | 2.63E−09 |
| 164 | serine (or cysteine) proteinase inhibitor, clade C (antithrombin), memb | 4502261 | 2 | 1.58E−04 |
| 165 | apolipoprotein A-IV precursor [Homo sapiens] | 4502151 | 4 | 4.20E−11 |
| 166 | apolipoprotein A-II precursor [Homo sapiens] | 4502149 | 4 | 1.72E−06 |
| 167 | alpha-1-microglobulin/bikunin precursor; Alpha-1-microglobulin/bikunin | 4502067 | 1 | 8.33E−11 |
| 168 | albumin precursor; PRO0883 protein [Homo sapiens] | 4502027 | 103 | 1.33E−14 |
| 169 | alpha-2-HS-glycoprotein; Alpha-2HS-glycoprotein [Homo sapiens] | 4502005 | 1 | 4.23E−03 |
| 170 | afamin precursor; alpha-albumin [Homo sapiens] | 4501987 | 1 | 1.10E−12 |
| 171 | alpha-1-antichymotrypsin, precursor; alpha-1-antichymotrypsin; antichym | 4501843 | 1 | 1.19E−05 |

TABLE 3.4
Proteins identified in vitreous fluid (IV)

| | | | | Probability of |
|---|---|---|---|---|
| | | | Peptide | randomized |
| | Protein | Accession number | Hits | identification |
| 1 | PREDICTED: similar to Piccolo protein (Aczonin) [Homo sapiens] | 51492882 | 1 | 9.11E−03 |
| 2 | PREDICTED: similar to phosducin-like 3; phosducin-like 2; IAP-associat | 51492730 | 1 | 9.80E−05 |
| 3 | PREDICTED: hypothetical protein XP_498867 [Homo sapiens] | 51475312 | 1 | 1.37E−03 |
| 4 | PREDICTED: KIAA0179 [Homo sapiens] | 51475296 | 1 | 8.94E−04 |
| 5 | PREDICTED: 5-azacytidine induced 1 [Homo sapiens] | 51474428 | 1 | 9.17E−03 |
| 6 | PREDICTED: FLJ46675 protein [Homo sapiens] | 51474152 | 1 | 6.48E−03 |
| 7 | PREDICTED: similar to Golgi autoantigen, golgin subfamily A member 6 ( | 51472431 | 1 | 5.11E−03 |
| 8 | PREDICTED: similar to centaurin, gamma-like family, member 1; ARF GTPa | 51468155 | 1 | 6.34E−03 |
| 9 | PREDICTED: similar to ribosomal protein L31 [Homo sapiens] | 51466239 | 1 | 5.81E−04 |
| 10 | PREDICTED: hypothetical protein XP_498937 [Homo sapiens] | 51463864 | 1 | 3.47E−03 |
| 11 | PREDICTED: cardiomyopathy associated 3 [Homo sapiens] | 51460893 | 1 | 5.89E−05 |
| 12 | M-phase phosphoprotein 1; mitotic kinesin-like protein [Homo sapiens] | 46049114 | 1 | 4.11E−03 |
| 13 | bifunctional phosphopantetheine adenylyl transferase/dephospho CoA kin | 46048207 | 1 | 7.22E−03 |
| 14 | hypothetical protein FLJ31322 [Homo sapiens] | 45387953 | 1 | 1.29E−03 |
| 15 | mitofusin 1 isoform 1; putative transmembrane GTPase [Homo sapiens] | 45269137 | 1 | 9.39E−03 |
| 16 | KIAA0317 [Homo sapiens] | 42734315 | 1 | 5.58E−03 |
| 17 | clusterin isoform 1; complement-associated protein SP-40 [Homo sapiens | 42716297 | 4 | 1.66E−10 |
| 18 | PREDICTED: hypothetical protein XP_376099 [Homo sapiens] | 42656388 | 1 | 6.07E−03 |
| 19 | nuclear receptor coactivator 7; estrogen receptor associated protein 1 | 42476175 | 1 | 6.25E−03 |
| 20 | semaphorin 3D; collapsin 2 [Homo sapiens] | 41406086 | 1 | 5.79E−04 |
| 21 | importin 13; Ran binding protein 13; karyopherin 13 [Homo sapiens] | 41281425 | 1 | 1.49E−04 |
| 22 | PREDICTED: hypothetical protein XP_373740 [Homo sapiens] | 41222847 | 2 | 1.59E−03 |
| 23 | U5 snRNP-specific protein, 116 kD [Homo sapiens] | 41152056 | 1 | 1.88E−03 |
| 24 | PREDICTED: hypothetical protein LOC285335 [Homo sapiens] | 41146536 | 1 | 2.58E−03 |
| 25 | dickkopf homolog 3; RIG-like 7-1; RIG-like 5-6; dickkopf (Xenopus laev | 40548389 | 1 | 2.56E−04 |
| 26 | retinitis pigmentosa 1-like 1 [Homo sapiens] | 40255278 | 1 | 5.54E−03 |
| 27 | hypothetical protein LOC168850 [Homo sapiens] | 39753953 | 1 | 6.17E−03 |
| 28 | serine (or cysteine) proteinase inhibitor, clade F (alpha-2 antiplasmi | 39725934 | 6 | 1.03E−08 |
| 29 | FLJ25005 protein [Homo sapiens] | 38570111 | 1 | 6.94E−03 |
| 30 | pancreatic ribonuclease precursor; RNase upl-1 [Homo sapiens] | 38201682 | 1 | 1.20E−04 |
| 31 | gelsolin isoform b [Homo sapiens] | 38044288 | 2 | 7.08E−08 |
| 32 | sodium-dependent organic anion transporter [Homo sapiens] | 37537552 | 1 | 6.81E−05 |
| 33 | hypothetical protein DKFZp434O0527 [Homo sapiens] | 34916026 | 1 | 7.81E−04 |
| 34 | zinc finger protein 135 (clone pHZ-17); zinc finger protein 61 [Homo s | 34419633 | 1 | 8.75E−03 |
| 35 | hypothetical protein MGC35261 [Homo sapiens] | 33859793 | 1 | 1.07E−03 |
| 36 | transmembrane channel-like 7 [Homo sapiens] | 33636691 | 1 | 5.84E−03 |
| 37 | glycosyltransferase-like 1B; ortholog of mouse glycosyltransferase-lik | 33285008 | 1 | 9.33E−03 |
| 38 | cardiomyopathy associated 5; 2310076E16Rik [Homo sapiens] | 32698780 | 1 | 8.25E−03 |
| 39 | cytosolic ovarian carcinoma antigen 1 isoform b; APK1 antigen [Homo sa | 32528291 | 1 | 2.72E−03 |
| 40 | vitamin D-binding protein precursor; vitamin D-binding alpha-globulin | 32483410 | 4 | 1.49E−13 |
| 41 | cytosolic phosphoenolpyruvate carboxykinase 1; phosphoenolpyruvate car | 32483401 | 1 | 4.79E−03 |
| 42 | prostaglandin D2 synthase 21 kDa; prostaglandin D synthase; prostagland | 32171249 | 1 | 7.44E−07 |
| 43 | hypothetical protein FLJ38973 [Homo sapiens] | 31581541 | 1 | 1.93E−03 |
| 44 | eukaryotic translation initiation factor 2C, 3 isoform a; argonaute 3 | 29294647 | 1 | 3.35E−03 |
| 45 | notch4 preproprotein; Notch (Drosophila) homolog 4 [Homo sapiens] | 27894370 | 1 | 5.09E−04 |
| 46 | nucleoporin 210; nuclear pore membrane glycoprotein 210 [Homo sapiens] | 27477134 | 1 | 9.34E−03 |
| 47 | transcription factor-like protein 4 isoform gamma; MAX-like bHLHZIP pr | 24586667 | 1 | 8.39E−03 |
| 48 | serine/threonine kinase 36 (fused homolog, Drosophila); serine/threoni | 24308123 | 1 | 2.26E−03 |
| 49 | cytoplasmic FMR1 interacting protein 1; selective hybridizing clone (m | 24307969 | 1 | 1.25E−03 |
| 50 | zinc finger protein 560 [Homo sapiens] | 22749003 | 1 | 1.43E−03 |
| 51 | junctophilin 1; mitsugumin72; junctophilin type1 [Homo sapiens] | 21735575 | 1 | 2.50E−03 |
| 52 | zinc finger protein 625 [Homo sapiens] | 21687161 | 1 | 6.05E−03 |
| 53 | chromosome 10 open reading frame 87; Em:AC061711.1 [Homo sapiens] | 21389373 | 1 | 4.19E−03 |
| 54 | PAP associated domain containing 1 [Homo sapiens] | 21361704 | 1 | 4.69E−04 |
| 55 | transient receptor potential cation channel, subfamily M, member 8 [Ho | 21361691 | 1 | 3.58E−04 |
| 56 | serine (or cysteine) proteinase inhibitor, clade A (alpha-1 antiprotei | 21361198 | 6 | 1.60E−10 |
| 57 | alpha 1B-glycoprotein [Homo sapiens] | 21071030 | 2 | 2.75E−07 |
| 58 | proprotein convertase subtilisin/kexin type 5 preproprotein; protease | 20336246 | 1 | 1.54E−04 |
| 59 | vitronectin precursor; serum spreading factor; somatomedin B; compleme | 18201911 | 1 | 4.77E−05 |
| 60 | ribosomal protein L10-like protein [Homo sapiens] | 18152783 | 1 | 1.59E−03 |
| 61 | ubiquitin specific protease 28 [Homo sapiens] | 16507200 | 1 | 8.91E−03 |
| 62 | E1A binding protein p400; CAGH32 protein; trinucleotide repeat contain | 15805014 | 1 | 4.47E−03 |
| 63 | postreplication repair protein hRAD18p; RAD18, S. cerevisiae, homolog | 14550405 | 1 | 2.76E−03 |
| 64 | hypothetical protein MGC10882 [Homo sapiens] | 14249240 | 1 | 6.22E−05 |
| 65 | hypothetical protein MGC4308 [Homo sapiens] | 14150167 | 1 | 7.84E−03 |
| 66 | dual specificity phosphatase 5; serine/threonine specific protein phos | 12707566 | 1 | 1.12E−04 |
| 67 | crystallin, beta A3; eye lens structural protein [Homo sapiens] | 12056461 | 3 | 2.72E−10 |
| 68 | SH3 and multiple ankyrin repeat domains 1; somatostatin receptor-inter | 11968152 | 1 | 8.27E−04 |
| 69 | fibrinogen, gamma chain isoform gamma-B precursor [Homo sapiens] | 11761633 | 1 | 3.10E−11 |
| 70 | potassium channel, subfamily K, member 12; tandem pore domain potassiu | 11545761 | 1 | 4.26E−04 |
| 71 | kallikrein 14 preproprotein; kallikrein-like protein 6 [Homo sapiens] | 11545747 | 1 | 3.25E−04 |
| 72 | supervillin isoform 2; membrane-associated F-actin binding protein p20 | 11496982 | 1 | 2.86E−03 |
| 73 | prosaposin (variant Gaucher disease and variant metachromatic leukodys | 11386147 | 1 | 1.13E−04 |
| 74 | succinate-CoA ligase, ADP-forming, beta subunit; SCS-betaA for ATP spe | 11321583 | 1 | 5.79E−03 |
| 75 | hemopexin [Homo sapiens] | 11321561 | 5 | 2.68E−05 |
| 76 | ATP-binding cassette, sub-family F, member 1; ATP-binding cassette 50; | 10947135 | 1 | 6.21E−03 |
| 77 | EGF-containing fibulin-like extracellular matrix protein 1 isoform b; f | 9665253 | 3 | 1.10E−10 |
| 78 | orosomucoid 1 precursor; alpha-1-acid glycoprotein 1; Orosomucoid-1 (al | 9257232 | 1 | 3.85E−08 |
| 79 | hypothetical protein FLJ20626 [Homo sapiens] | 8923582 | 1 | 3.88E−03 |
| 80 | hypothetical protein FLJ20580 [Homo sapiens] | 8923541 | 1 | 8.20E−04 |
| 81 | crystallin, gamma S; crystallin, gamma 8 [Homo sapiens] | 8922120 | 2 | 1.57E−11 |
| 82 | cleavage and polyadenylation specific factor 3, 73 kDa; cleavage and pol | 7706427 | 1 | 1.10E−03 |
| 83 | glyceraldehyde-3-phosphate dehydrogenase [Homo sapiens] | 7669492 | 1 | 7.38E−07 |
| 84 | GTPase regulator associated with the focal adhesion kinase pp125; GTPas | 7662208 | 1 | 1.08E−03 |
| 85 | opticin; opticin, oculoglycan; oculoglycan [Homo sapiens] | 7657419 | 1 | 3.15E−09 |
| 86 | plasma glutathione peroxidase 3 precursor [Homo sapiens] | 6006001 | 2 | 6.81E−10 |
| 87 | Wnt inhibitory factor-1 precursor; Wnt inhibitory factor-1 [Homo sapien | 6005950 | 1 | 8.17E−04 |
| 88 | RBP4 gene product [Homo sapiens] | 5803139 | 1 | 1.46E−03 |
| 89 | tripeptidyl-peptidase I precursor [Homo sapiens] | 5729770 | 1 | 3.73E−05 |
| 90 | translocase of inner mitochondrial membrane 23 (yeast) homolog; translo | 5454122 | 1 | 5.15E−03 |
| 91 | structural maintenance of chromosomes 2-like 1; structural maintenance | 5453591 | 1 | 2.58E−03 |
| 92 | nebulin [Homo sapiens] | 4758794 | 1 | 9.60E−03 |
| 93 | dual specificity phosphatase 8; serine/threonine specific protein phosp | 4758212 | 1 | 4.25E−05 |
| 94 | beta-2-microglobulin precursor [Homo sapiens] | 4757826 | 2 | 3.04E−08 |
| 95 | phosphodiesterase 6A, alpha subunit [Homo sapiens] | 4585864 | 1 | 2.08E−04 |
| 96 | transferrin; PRO2086 protein [Homo sapiens] | 4557871 | 34 | 8.64E−13 |
| 97 | galactosylceramidase precursor; galactocerebrosidase; Galactosylceramin | 4557613 | 1 | 4.63E−04 |
| 98 | ceruloplasmin (ferroxidase); Ceruloplasmin [Homo sapiens] | 4557485 | 4 | 2.10E−08 |
| 99 | complement component 3 precursor; acylation-stimulating protein cleavag | 4557385 | 4 | 1.12E−09 |
| 100 | complement component 1 inhibitor precursor [Homo sapiens] | 4557379 | 1 | 3.36E−07 |
| 101 | beta-2-glycoprotein I precursor [Homo sapiens] | 4557327 | 1 | 3.73E−08 |
| 102 | apolipoprotein E precursor; apolipoprotein E3 [Homo sapiens] | 4557325 | 2 | 4.77E−09 |
| 103 | apolipoprotein A-I precursor [Homo sapiens] | 4557321 | 6 | 2.84E−09 |
| 104 | alpha-2-macroglobulin precursor [Homo sapiens] | 4557225 | 1 | 6.33E−04 |
| 105 | chitinase 3-like 1; cartilage glycoprotein-39 [Homo sapiens] | 4557018 | 1 | 9.78E−06 |
| 106 | transthyretin; prealbumin [Homo sapiens] | 4507725 | 8 | 8.88E−15 |
| 107 | SRY (sex determining region Y)-box 4; SRY-related HMG-box gene 4; ecotr | 4507163 | 1 | 5.28E−03 |
| 108 | ribosomal protein S4, Y-linked 1 Y isoform; 40S ribosomal protein S4, Y | 4506727 | 1 | 8.87E−05 |
| 109 | RBP3 gene product [Homo sapiens] | 4506453 | 4 | 1.63E−09 |
| 110 | plasminogen [Homo sapiens] | 4505881 | 1 | 2.12E−04 |
| 111 | orosomucoid 2; alpha-1-acid glycoprotein, type 2 [Homo sapiens] | 4505529 | 2 | 4.67E−06 |
| 112 | kininogen 1; alpha-2-thiol proteinase inhibitor; bradykinin [Homo sapie | 4504893 | 1 | 3.72E−04 |
| 113 | I factor (complement) [Homo sapiens] | 4504579 | 1 | 9.70E−07 |
| 114 | histidine-rich glycoprotein precursor; histidine-proline rich glycoprot | 4504489 | 1 | 5.05E−08 |
| 115 | beta globin; hemoglobin beta chain [Homo sapiens] | 4504349 | 2 | 3.04E−07 |
| 116 | alpha 2 globin; alpha globin; alpha-2 globin [Homo sapiens] | 4504345 | 1 | 4.25E−04 |
| 117 | cathepsin D preproprotein [Homo sapiens] | 4503143 | 2 | 8.90E−04 |
| 118 | crystallin, beta B2; eye lens structural protein [Homo sapiens] | 4503063 | 1 | 5.59E−06 |
| 119 | crystallin, beta B1; eye lens structural protein [Homo sapiens] | 4503061 | 6 | 5.62E−08 |
| 120 | crystallin, alpha B; heat-shock 20 kD like-protein [Homo sapiens] | 4503057 | 2 | 1.53E−06 |
| 121 | crystallin, alpha A; crystallin, alpha-1; human alphaA-crystallin (CRYA | 4503055 | 4 | 5.90E−05 |
| 122 | cholinergic receptor, nicotinic, alpha polypeptide 2 (neuronal) [Homo s | 4502823 | 1 | 3.30E−03 |
| 123 | centromere protein C 1; Centromere autoantigen C1 [Homo sapiens] | 4502779 | 1 | 1.23E−03 |
| 124 | complement component 4B proprotein [Homo sapiens] | 4502501 | 6 | 2.35E−06 |
| 125 | complement factor B preproprotein; C3 proactivator; C3 proaccelerator; | 4502397 | 2 | 6.20E−08 |
| 126 | serine (or cysteine) proteinase inhibitor, clade C (antithrombin), memb | 4502261 | 3 | 1.03E−06 |
| 127 | apolipoprotein A-II precursor [Homo sapiens] | 4502149 | 1 | 1.39E−06 |
| 128 | albumin precursor; PRO0883 protein [Homo sapiens] | 4502027 | 66 | 3.00E−14 |
| 129 | alpha-2-HS-glycoprotein; Alpha-2HS-glycoprotein [Homo sapiens] | 4502005 | 1 | 3.79E−04 |
| 130 | alkylglycerone phosphate synthase precursor [Homo sapiens] | 4501993 | 1 | 5.09E−03 |
| 131 | afamin precursor; alpha-albumin [Homo sapiens] | 4501987 | 1 | 4.49E−07 |
| 132 | alpha-1-antichymotrypsin, precursor; alpha-1-antichymotrypsin: antichym | 4501843 | 1 | 2.45E−04 |

TABLE 3.5
Vitreous proteins identified in three ISS patients by total vitrectromy (V)

| | Reference | Accession | MW | P (pro) | Patients |
|---|---|---|---|---|---|
| 1 | Serum albumin precursor | P02768 | 69321.63 | 1.00E−30 | CC, TR, NT |
| 2 | Alpha 2 globin variant (Fragment) | Q53F97 | 15270.94 | 1.00E−30 | CC, TR, NT |
| 3 | Serotransferrin precursor | P02787 | 76999.66 | 1.44E−14 | CC, TR, NT |
| 1 | Hemoglobin delta subunit | P02042 | 15914.25 | 3.72E−13 | CC, TR, NT |
| 2 | Alpha-1-acid glycoprotein 1 precursor | P02763 | 23496.77 | 3.08E−10 | CC, TR, NT |
| 3 | IGLC2 protein | Q567P1 | 24784.13 | 1.05E−11 | CC, TR, NT |
| 4 | Clusterin precursor | P10909 | 52461.05 | 1.87E−08 | CC, TR, NT |
| 5 | Beta-globin gene from a thalassemia patient | Q14473 | 18918.59 | 1.67E−14 | CC, TR, NT |
| 6 | Hypothetical protein FLJ16039 | Q6ZNI2 | 79911.39 | 1.27E−03 | CC, TR, NT |
| 7 | Voltage-dependent N-type calcium channel alpha-1B subunit | Q00975 | 262326.3 | 1.38E−04 | CC, TR, NT |
| 8 | Transthyretin precursor | P02766 | 15877.05 | 1.00E−30 | TR, CC |
| 9 | Ig gamma-1 chain C region | P01857 | 36083.16 | 2.68E−13 | TR, CC |
| 10 | Pigment epithelium-derived factor precursor | P36955 | 46313.36 | 2.54E−12 | CC, TR |
| 11 | Collagen alpha 1(II) chain precursor | P02458 | 134410.6 | 2.28E−07 | TR, CC |
| 12 | Hemopexin precursor | P02790 | 51643.32 | 1.00E−30 | CC, TR |
| 13 | Alpha-2-macroglobulin precursor | P01023 | 163174.3 | 5.92E−13 | CC, TR |
| 14 | Alpha-1-antitrypsin precursor | P01009 | 46707.09 | 3.42E−13 | CC, TR |
| 15 | Interphotoreceptor retinoid-binding protein precursor | P10745 | 135277.8 | 3.33E−15 | TR, CC |
| 16 | Hypothetical protein | Q569I7 | 20655.36 | 9.77E−14 | TR, CC |
| 17 | Hypothetical protein DKFZp686I04196 (Fragment) | Q6N093 | 46031.66 | 1.15E−10 | TR, CC |
| 18 | Opticin precursor (Oculoglycan) | Q9UBM4 | 37237.39 | 1.70E−13 | CC, TR |
| 19 | Complement C4-A precursor | P0C0L4 | 192649.5 | 3.64E−13 | CC, TR |
| 20 | IGKC protein | Q502W4 | 25919.92 | 1.00E−30 | CC, TR |
| 21 | Glutathione peroxidase 3 precursor | P22352 | 25488.96 | 1.97E−09 | CC, TR |
| 22 | Prostaglandin-H2 D-isomerase precursor | P41222 | 21015.35 | 8.44E−14 | CC, TR |
| 23 | Apolipoprotein A-I precursor | P02647 | 30758.94 | 3.33E−09 | CC, TR |
| 24 | Vitamin D-binding protein | Q6GTG1 | 52902.04 | 8.22E−14 | CC, TR |
| 25 | VH1 protein precursor (Fragment) | O95978 | 17292.5 | 1.44E−07 | CC, TR |
| 26 | Hypothetical protein DKFZp686G2174 | Q63HK5 | 118419.6 | 2.46E−03 | TR, CC |
| 27 | Apolipoprotein A-II precursor | P02652 | 11167.9 | 7.31E−11 | CC, TR |
| 28 | Keratin, type I cytoskeletal 9 | P35527 | 62091.76 | 1.00E−30 | TR, CC |
| 29 | Haptoglobin precursor | P00738 | 45176.59 | 6.64E−10 | TR, CC |
| 30 | Ceruloplasmin precursor | P00450 | 122127.6 | 5.53E−08 | TR, CC |
| 31 | Alpha crystallin B chain | P02511 | 20146.43 | 1.80E−07 | NT, CC |
| 32 | Dickkopf-related protein 3 precursor | Q9UBP4 | 38266.07 | 1.29E−05 | TR, CC |
| 33 | Ig gamma-3 chain C region | P01860 | 32309.81 | 3.71E−13 | CC, TR |
| 34 | Adenosine A3 receptor | P33765 | 36160.19 | 1.70E−03 | CC, NT |
| 35 | Solute carrier organic anion transporter family member 5A1 | Q9H2Y9 | 91818.61 | 2.31E−03 | CC, TR |
| 36 | Complement C3 precursor | P01024 | 187045.3 | 1.11E−15 | CC |
| 37 | Keratin, type II cytoskeletal 1 | P04264 | 65846.88 | 3.59E−12 | TR |
| 38 | Carbonic anhydrase 1 | P00915 | 28721.34 | 4.94E−13 | NT |
| 39 | Beta crystallin S | P22914 | 20861.95 | 4.62E−13 | NT |
| 40 | Peroxiredoxin 2 | P32119 | 21747.2 | 1.00E−30 | NT |
| 41 | Alpha crystallin A chain | P02489 | 19896.9 | 6.75E−07 | NT |
| 42 | Osteopontin precursor | P10451 | 35401.25 | 9.00E−11 | CC |
| 43 | Keratin, type I cytoskeletal 10 | P13645 | 59482.76 | 1.49E−12 | TR |
| 44 | Keratin, type II cytoskeletal 2 | P35908 | 65825.37 | 5.53E−08 | TR |
| 45 | Calcineurin-binding protein Cabin 1 | Q9Y6J0 | 246194.6 | 3.19E−03 | NT |
| 46 | Ig heavy chain V-III region | P01764 | 12574.22 | 9.50E−08 | TR |
| 47 | Beta crystallin B2 | P43320 | 23234.38 | 8.03E−11 | NT |
| 48 | Alpha-1B-glycoprotein precursor | P04217 | 54238.7 | 1.51E−08 | CC |
| 49 | Integrin alpha-5 precursor | P08648 | 114464.9 | 5.43E−04 | NT |
| 50 | Alpha-1-acid glycoprotein 2 precursor | P19652 | 23587.64 | 4.04E−13 | CC |
| 51 | Peroxiredoxin 1 | Q06830 | 22096.28 | 4.12E−06 | NT |
| 52 | Hypothetical protein DKFZp781M0386 | Q5CZ94 | 24986.3 | 9.70E−05 | CC |
| 53 | Hypothetical protein DKFZp686M24218 | Q6MZX7 | 52387.13 | 3.02E−13 | CC |
| 54 | NIR3 | Q9BZ72 | 148839.5 | 4.48E−06 | NT |
| 55 | Plasma protease C1 inhibitor precursor | P05155 | 55119.49 | 1.54E−04 | CC, TR |
| 56 | Calmodulin binding transcription activator 1 | Q5VUE1 | 183554.8 | 1.46E−03 | TR, CC |
| 57 | Hypothetical protein FLJ16692 | Q6ZMT6 | 16180.33 | 1.09E−03 | CC, TR |
| 58 | Haptoglobin-related protein precursor | P00739 | 38982.66 | 8.59E−07 | CC |
| 59 | Cystatin C precursor | P01034 | 15789.08 | 2.31E−04 | CC |
| 60 | tRNA (guanine-N(7)-)-methyltransferase | Q9UBP6 | 31451.11 | 3.95E−05 | NT |
| 61 | Calsyntenin-1 precursor | O94985 | 109723.7 | 1.10E−05 | CC |
| 62 | Carbonic anhydrase 2 | P00918 | 29096.89 | 2.58E−05 | NT |
| 63 | Amyloid-like protein 2 precursor | Q06481 | 86900.28 | 5.97E−03 | CC |
| 64 | Elastic titin (Fragment) | Q10465 | 882501.8 | 6.73E−04 | CC |
| 65 | Collagen alpha 2(IX) chain precursor | Q14055 | 65090.96 | 6.00E−08 | TR |
| 66 | Hypothetical protein DKFZp686J11235 (Fragment) | Q6MZW0 | 54424.95 | 1.06E−05 | CC |
| 67 | L-lactate dehydrogenase A-like 6A | Q6ZMR3 | 36484.2 | 2.33E−04 | NT |
| 68 | Spectrin alpha chain, erythrocyte | P02549 | 279742.3 | 2.09E−05 | NT |
| 69 | Plasma retinol-binding protein precursor | P02753 | 22995.26 | 1.33E−13 | TR |
| 70 | Tripeptidyl-peptidase I precursor | O14773 | 61190.67 | 1.00E−30 | CC |
| 71 | Toll-like receptor 3 precursor | O15455 | 103762.9 | 4.01E−03 | NT |
| 72 | SART-1 (Squamous cell carcinoma antigen recognized by T cells 1) | O43290 | 90200.29 | 8.43E−03 | TR |
| 73 | KIAA0425 protein (Fragment) | O43308 | 141056.5 | 1.74E−03 | NT |
| 74 | Chondromodulin-I precursor | O75829 | 37078.55 | 5.16E−10 | TR |
| 75 | Arginyl-tRNA--protein transferase 1 | O95260 | 59052.28 | 6.80E−03 | CC |
| 76 | Beta-tubulin cofactor D | O95458 | 138562.4 | 6.55E−03 | CC |
| 77 | Prothrombin precursor | P00734 | 69992.2 | 1.44E−04 | TR |
| 78 | Complement factor B precursor | P00751 | 85478.58 | 5.53E−04 | CC |
| 79 | Alpha-1-antichymotrypsin precursor | P01011 | 47620.63 | 9.77E−06 | CC |
| 80 | Angiotensinogen precursor | P01019 | 53120.61 | 5.32E−05 | CC |
| 81 | Keratin, type I cytoskeletal 14 | P02533 | 51458.45 | 2.83E−04 | TR |
| 82 | Apolipoprotein E precursor (Apo-E) | P02649 | 36131.79 | 8.83E−07 | CC |
| 83 | Fibrinogen gamma chain precursor | P02679 | 51478.88 | 4.23E−05 | CC |
| 84 | Band 3 anion transport protein | P02730 | 101727.8 | 1.72E−06 | NT |
| 85 | AMBP protein precursor | P02760 | 38973.99 | 6.52E−04 | CC |
| 86 | Catalase | P04040 | 59587.83 | 3.92E−04 | NT |
| 87 | Krueppel-related zinc finger protein 2 | P10073 | 12059.54 | 4.92E−03 | NT |
| 88 | Versican core protein precursor | P13611 | 372589 | 2.27E−06 | CC |
| 89 | Guanine nucleotide-binding protein | P19087 | 40019.09 | 2.61E−04 | TR |
| 90 | Inter-alpha-trypsin inhibitor heavy chain H2 precursor | P19823 | 106369.8 | 2.75E−03 | CC |
| 91 | Inter-alpha-trypsin inhibitor heavy chain H1 precursor | P19827 | 101325.8 | 3.23E−03 | TR |
| 92 | Large proline-rich protein BAT3 | P46379 | 119334.2 | 3.20E−04 | NT |
| 93 | Cadherin-8 precursor | P55286 | 88198.66 | 1.68E−03 | CC |
| 94 | Low-density lipoprotein receptor-related protein 2 precursor | P98164 | 521587.6 | 3.27E−05 | CC |
| 95 | Galectin-3 binding protein precursor | Q08380 | 65289.4 | 8.46E−07 | TR |
| 96 | EGF-containing fibulin-like extracellular matrix protein 1 precursor | Q12805 | 54604.29 | 1.62E−03 | CC |
| 97 | Ectonucleotide pyrophosphatase/phosphodiesterase 2 | Q13822 | 98939.61 | 1.61E−04 | CC |
| 98 | Keratin 10 | Q14664 | 57212.99 | 8.68E−03 | TR |
| 99 | Hypothetical protein FLJ25351 | Q53TS8 | 71114 | 4.40E−04 | TR |
| 100 | KIAA0467 protein | Q5T011 | 230969.8 | 3.37E−03 | CC |
| 101 | OTTHUMP00000018325 | Q5VW09 | 53975.58 | 8.38E−04 | CC |
| 102 | LOC150763 protein | Q6NUI2 | 87507.18 | 9.17E−05 | CC |
| 103 | Hypothetical protein | Q6PHR9 | 9188.304 | 2.03E−03 | CC |
| 104 | TROAP protein | Q6PJU7 | 15883.54 | 5.01E−03 | CC |
| 105 | QMDP9344 | Q6UXP3 | 13598.86 | 6.72E−03 | CC |
| 106 | Nicotinate phosphoribosyltransferase-like protein | Q6XQN6 | 60239.51 | 6.56E−03 | TR |
| 107 | Hypothetical protein FLJ26301 | Q6ZP85 | 25787.31 | 7.38E−03 | TR |
| 108 | Hypothetical protein FLJ46155 | Q6ZRR8 | 15002.66 | 2.28E−03 | TR |
| 109 | ABC A13 | Q86UQ4 | 575811.8 | 5.71E−03 | NT |
| 110 | FLJ35848 protein (Fragment) | Q8IVZ7 | 41676.67 | 4.20E−03 | NT |
| 111 | Hypothetical protein FLJ38687 | Q8N8Z1 | 16623.52 | 1.77E−04 | TR |
| 112 | Unc-13 homolog C | Q8NB66 | 135534.9 | 3.92E−03 | TR |
| 113 | Hypothetical protein DKFZp434D011 | Q8NDL9 | 97472.62 | 2.27E−03 | CC |
| 114 | Tubulin tyrosine ligase-like protein 11 | Q8NHH1 | 57999.14 | 7.78E−03 | TR |
| 115 | CLL-associated antigen KW-2 | Q8TDG9 | 96521.61 | 2.07E−03 | TR |
| 116 | Hypothetical protein FLJ22955 | Q8WVC6 | 26533.31 | 6.76E−03 | CC |
| 117 | Very large G protein-coupled receptor 1b | Q8WXG9 | 692246.9 | 1.95E−03 | CC |
| 118 | Keratin, type I | Q92764 | 47566.21 | 9.34E−04 | TR |
| 119 | Secreted frizzled-related protein 3 precursor | Q92765 | 36230.29 | 5.24E−03 | TR |
| 120 | Hypothetical protein FLJ32955 | Q96LZ6 | 14450.25 | 1.39E−03 | NT |
| 121 | Olfactory receptor 4K3 | Q96R72 | 35354.45 | 7.73E−03 | NT |
| 122 | Protein LRP16 | Q9BQ69 | 35482.73 | 7.20E−04 | NT |
| 123 | DNA-directed RNA polymerase III | Q9BUI4 | 60573.35 | 1.78E−03 | CC |
| 124 | Glucose-6-phosphatase catalytic subunit 3 | Q9BUM1 | 38709.14 | 9.22E−03 | CC |
| 125 | Dynein intermediate chain 2 | Q9GZS0 | 68807.61 | 8.69E−06 | TR |
| 126 | Hypothetical protein DKFZp434F0116 | Q9H0J7 | 53323.07 | 1.44E−03 | NT |
| 127 | Presenilins associated rhomboid-like protein | Q9H300 | 42176.74 | 2.85E−03 | CC |
| 128 | Hypothetical protein FLJ12973 | Q9H967 | 69709.27 | 5.73E−03 | TR |
| 129 | Sideroflexin-1 | Q9H9B4 | 35465.31 | 1.82E−03 | TR |
| 130 | Tumor necrosis factor receptor superfamily member 19 precursor | Q9NS68 | 45984.76 | 6.37E−04 | CC |
| 131 | Protein C6orf72 precursor | Q9NU53 | 36817.27 | 6.18E−04 | CC |
| 132 | Leucyl-tRNA synthetase | Q9P2J5 | 134379.5 | 8.03E−03 | NT |
| 133 | Neurochondrin-1 | Q9UBB6 | 78813.82 | 5.91E−03 | CC |
| 134 | MAx-like protein X | Q9UH92 | 33279.74 | 4.11E−03 | TR |
| 135 | Zinc finger transcription factor Trps1 | Q9UHF7 | 141432.2 | 5.45E−03 | CC |
| 136 | Wnt inhibitory factor 1 precursor | Q9Y5W5 | 41499.8 | 7.84E−05 | CC |
| 137 | Mitotic spindle assembly checkpoint protein MAD1 | Q9Y6D9 | 83015.71 | 4.43E−03 | CC |
| 138 | Piccolo protein (Aczonin) | Q9Y6V0 | 566313.1 | 7.26E−03 | CC |

TABLE 4
Proteins identified in vitreous fluids of cadaver samples (IV)

| | Protein | Accession | MW | P (pro) | Patient ID |
|---|---|---|---|---|---|
| 1 | Serum albumin precursor | P02768 | 69321.63 | 1.00E−30 | 0541; 0535; 0560; |
| | | | | | 0556; 0567; 0571; |
| | | | | | 0552; 0578; 0549; |
| | | | | | 0574 |
| 2 | Transthyretin precursor | P02766 | 15877.05 | 1.00E−30 | 0571; 0571; 0549; |
| | | | | | 0574; 0535; 0567; |
| | | | | | 0560; 0578; 0556; |
| | | | | | 0552 |
| 3 | Serotransferrin precursor | P02787 | 76999.66 | 1.11E−15 | 0541; 0535; 0541; |
| | | | | | 0556; 0552; 0574; |
| | | | | | 0549; 0560; 0578; |
| | | | | | 0571 |
| 4 | Alpha-1-antitrypsin precursor | P01009 | 46707.09 | 1.00E−30 | 0560; 0535; 0556; |
| | | | | | 0549; 0571; 0574; |
| | | | | | 0578; 0560; 0567; |
| | | | | | 0541 |
| 5 | Alpha-1-acid glycoprotein 1 precursor | P02763 | 23496.77 | 5.14E−13 | 0535; 0535; 0549; |
| | | | | | 0578; 0556; 0552; |
| | | | | | 0560; 0574; 0541; |
| | | | | | 0567 |
| 6 | Ig gamma-1 chain C region | P01857 | 36083.16 | 1.11E−15 | 0556; 0535; 0549; |
| | | | | | 0556; 0552; 0574; |
| | | | | | 0567; 0571; 0578; |
| | | | | | 0560 |
| 7 | Clusterin precursor | P10909 | 52461.05 | 3.36E−13 | 0574; 0567; 0535; |
| | | | | | 0556; 0549; 0578; |
| | | | | | 0541; 0571; 0560; |
| | | | | | 0574 |
| 8 | Ceruloplasmin precursor | P00450 | 122127.6 | 7.77E−15 | 0541; 0549; 0578; |
| | | | | | 0571; 0567; 0535; |
| | | | | | 0541; 0574; 0556; |
| | | | | | 0552 |
| 9 | Pigment epithelium-derived factor precursor | P36955 | 46313.36 | 1.65E−13 | 0541; 0535; 0571; |
| | | | | | 0549; 0541; 0578; |
| | | | | | 0560; 0552; 0567; |
| | | | | | 0574 |
| 10 | Apolipoprotein E precursor | P02649 | 36131.79 | 6.22E−14 | 0541; 0574; 0571; |
| | | | | | 0578; 0549; 0560; |
| | | | | | 0567; 0556; 0541; |
| | | | | | 0552 |
| 11 | Complement C4-A precursor | P0C0L4 | 192649.5 | 1.52E−13 | 0556; 0535; 0556; |
| | | | | | 0549; 0541; 0571; |
| | | | | | 0560; 0567; 0552; |
| | | | | | 0578 |
| 12 | Interphotoreceptor retinoid-binding protein precursor | P10745 | 135277.8 | 1.00E−30 | 0541; 0578; 0549; |
| | | | | | 0571; 0567; 0552; |
| | | | | | 0556; 0541; 0560 |
| 13 | Cystatin C precursor | P01034 | 15789.08 | 4.43E−13 | 0567; 0560; 0567; |
| | | | | | 0571; 0574; 0552; |
| | | | | | 0556; 0541; 0535 |
| 14 | Vitamin D-binding protein | Q6GTG1 | 52902.04 | 9.99E−15 | 0552; 0578; 0556; |
| | | | | | 0549; 0560; 0552; |
| | | | | | 0541; 0571; 0567; |
| | | | | | 0574 |
| 15 | Plasma protease C1 inhibitor precursor | P05155 | 55119.49 | 1.28E−11 | 0567; 0541; 0535; |
| | | | | | 0571; 0549; 0567; |
| | | | | | 0574; 0578; 0552; |
| | | | | | 0560 |
| 16 | Beta-2-microglobulin precursor | P61769 | 13705.91 | 1.11E−16 | 0556; 0560; 0535; |
| | | | | | 0552; 0556; 0574; |
| | | | | | 0567; 0549; 0578; |
| | | | | | 0571 |
| 17 | Hemopexin precursor | P02790 | 51643.32 | 1.22E−14 | 0541; 0552; 0560; |
| | | | | | 0549; 0574; 0535; |
| | | | | | 0578; 0571; 0541 |
| 18 | Alpha-2-macroglobulin precursor | P01023 | 163174.3 | 1.11E−15 | 0552; 0560; 0571; |
| | | | | | 0578; 0567; 0549; |
| | | | | | 0535; 0574; 0541; |
| | | | | | 0556 |
| 19 | IGKC protein | Q502W4 | 25919.92 | 1.22E−14 | 0571; 0552; 0574; |
| | | | | | 0549; 0560; 0556; |
| | | | | | 0535; 0571; 0567 |
| 20 | IGLC2 protein | Q567P1 | 24784.13 | 7.77E−14 | 0552; 0571; 0552; |
| | | | | | 0578; 0560; 0556; |
| | | | | | 0567; 0549; 0574; |
| | | | | | 0541 |
| 21 | Osteopontin precursor | P10451 | 35401.25 | 2.22E−14 | 0541; 0549; 0556; |
| | | | | | 0571; 0578; 0552; |
| | | | | | 0574; 0567; 0560 |
| 22 | Dickkopf-related protein 3 precursor | Q9UBP4 | 38266.07 | 1.11E−15 | 0567; 0535; 0578; |
| | | | | | 0541; 0556; 0567; |
| | | | | | 0549; 0574; 0560 |
| 23 | Complement C3 precursor | P01024 | 187045.3 | 2.11E−14 | 0552; 0549; 0567; |
| | | | | | 0574; 0560; 0571; |
| | | | | | 0541; 0552; 0535; |
| | | | | | 0578 |
| 24 | Alpha-1-antichymotrypsin precursor | P01011 | 47620.63 | 1.22E−14 | 0574; 0556; 0578; |
| | | | | | 0574; 0541; 0560; |
| | | | | | 0571; 0549; 0552 |
| 25 | Prostaglandin-H2 D-isomerase precursor | P41222 | 21015.35 | 1.00E−30 | 0567; 0541; 0552; |
| | | | | | 0549; 0574; 0578; |
| | | | | | 0571; 0556 |
| 26 | Alpha-1-acid glycoprotein 2 precursor | P19652 | 23587.64 | 3.33E−15 | 0552; 0535; 0549; |
| | | | | | 0578; 0552; 0556; |
| | | | | | 0571; 0574; 0560; |
| | | | | | 0567 |
| 27 | EGF-containing fibulin-like extracellular matrix protein 1 precursor | Q12805 | 54604.29 | 3.44E−12 | 0541; 0541; 0549; |
| | | | | | 0571; 0567; 0560; |
| | | | | | 0535; 0574; 0578; |
| | | | | | 0556 |
| 28 | Wnt inhibitory factor 1 precursor | Q9Y5W5 | 41499.8 | 7.44E−09 | 0567; 0578; 0560; |
| | | | | | 0567; 0535; 0541; |
| | | | | | 0549; 0552; 0556 |
| 29 | Ig gamma-3 chain C region | P01860 | 32309.81 | 3.82E−10 | 0578; 0571; 0549; |
| | | | | | 0567; 0552; 0541; |
| | | | | | 0574; 0556; 0560 |
| 30 | OTTHUMP00000018139 (Fragment) | Q5T2J8 | 97147.08 | 9.78E−05 | 0535; 0571; 0578; |
| | | | | | 0549; 0567; 0535; |
| | | | | | 0574; 0541; 0556; |
| | | | | | 0560 |
| 31 | Glutathione peroxidase 3 precursor | P22352 | 25488.96 | 8.40E−13 | 0574; 0556; 0552; |
| | | | | | 0541; 0549; 0574; |
| | | | | | 0571; 0567; 0560 |
| 32 | Apolipoprotein A-I precursor | P02647 | 30758.94 | 2.00E−14 | 0535; 0556; 0541; |
| | | | | | 0552; 0571; 0535; |
| | | | | | 0574; 0567; 0560 |
| 33 | Opticin precursor (Oculoglycan) | Q9UBM4 | 37237.39 | 2.44E−14 | 0567; 0578; 0552; |
| | | | | | 0574; 0560; 0571; |
| | | | | | 0556; 0549; 0541 |
| 34 | Amyloid-like protein 2 precursor | Q06481 | 86900.28 | 1.42E−11 | 0574; 0571; 0541; |
| | | | | | 0567; 0560; 0552; |
| | | | | | 0578 |
| 35 | Gelsolin precursor | P06396 | 85644.25 | 1.00E−30 | 0567; 0556; 0549; |
| | | | | | 0567; 0571; 0574; |
| | | | | | 0578; 0541; 0560 |
| 36 | Rho-GTPase-activating protein 26 | Q9UNA1 | 92176.67 | 3.74E−04 | 0552; 0549; 0571; |
| | | | | | 0560; 0574; 0541; |
| | | | | | 0552; 0578; 0556 |
| 37 | Glyceraldehyde-3-phosphate dehydrogenase | P04406 | 35899.36 | 2.10E−12 | 0560; 0549; 0578; |
| | | | | | 0556; 0574; 0552; |
| | | | | | 0571; 0541 |
| 38 | Haptoglobin precursor | P00738 | 45176.59 | 5.90E−10 | 0556; 0552; 0549; |
| | | | | | 0541; 0535; 0567; |
| | | | | | 0560; 0578 |
| 39 | Lysozyme C precursor | P61626 | 16526.29 | 1.00E−30 | 0571; 0552; 0560; |
| | | | | | 0574; 0541; 0549 |
| 40 | Von Ebner's gland protein precursor | P31025 | 19237.81 | 5.66E−14 | 0571; 0574; 0560; |
| | | | | | 0552; 0549; 0578 |
| 41 | Apolipoprotein A-II precursor | P02652 | 11167.9 | 1.66E−11 | 0574; 0552; 0556; |
| | | | | | 0535; 0541; 0560; |
| | | | | | 0567 |
| 42 | Fibrinogen alpha chain precursor | P02671 | 94914.27 | 1.37E−12 | 0552; 0535; 0560; |
| | | | | | 0578; 0549; 0541; |
| | | | | | 0571 |
| 43 | Haptoglobin-related protein precursor | P00739 | 38982.66 | 9.25E−10 | 0556; 0541; 0552; |
| | | | | | 0549; 0535; 0567 |
| 44 | Angiotensinogen precursor | P01019 | 53120.61 | 2.27E−12 | 0556; 0552; 0571; |
| | | | | | 0560; 0541; 0567; |
| | | | | | 0574 |
| 45 | Lysosomal trafficking regulator | Q99698 | 428865.6 | 8.85E−06 | 0574; 0578; 0567; |
| | | | | | 0560; 0556; 0541; |
| | | | | | 0571 |
| 46 | Carboxypeptidase | P16870 | 53117.17 | 5.48E−12 | 0567; 0541; 0571; |
| | | | | | 0574; 0578; 0552 |
| 47 | Hypothetical protein DKFZp686J11235 | Q6MZW0 | 54424.95 | 4.37E−13 | 0571; 0552; 0578; |
| | | | | | 0535; 0574 |
| 48 | Cathepsin D precursor | P07339 | 44523.66 | 8.15E−11 | 0552; 0556; 0549; |
| | | | | | 0560; 0571; 0552; |
| | | | | | 0578; 0574; 0567 |
| 49 | AMBP protein precursor | P02760 | 38973.99 | 8.60E−10 | 0574; 0535; 0549; |
| | | | | | 0552; 0560; 0541; |
| | | | | | 0578; 0567 |
| 50 | Complement factor B precursor | P00751 | 85478.58 | 1.75E−08 | 0549; 0567; 0552; |
| | | | | | 0541; 0556; 0535; |
| | | | | | 0560 |
| 51 | Beta-2-glycoprotein I precursor | P02749 | 38272.67 | 3.44E−14 | 0549; 0535; 0556; |
| | | | | | 0541; 0552; 0571 |
| 52 | Lactotransferrin precursor | P02788 | 78288.08 | 6.66E−15 | 0571; 0552; 0560; |
| | | | | | 0574; 0541 |
| 53 | Prolactin-inducible protein precursor | P12273 | 16561.8 | 3.17E−12 | 0560; 0552; 0571; |
| | | | | | 0574 |
| 54 | Neogenin precursor | Q92859 | 159859.3 | 3.55E−03 | 0578; 0541; 0578; |
| | | | | | 0549; 0571; 0560; |
| | | | | | 0556; 0552; 0574 |
| 55 | Alpha-actinin 3 | Q08043 | 103229.2 | 9.07E−04 | 0571; 0541; 0560; |
| | | | | | 0567; 0574; 0535; |
| | | | | | 0556; 0578 |
| 56 | Beta crystallin B2 | P43320 | 23234.38 | 3.11E−14 | 0549; 0574; 0541; |
| | | | | | 0556; 0560; 0535 |
| 57 | Hemoglobin delta subunit | P02042 | 15914.25 | 3.89E−14 | 0552; 0578; 0535; |
| | | | | | 0560; 0571 |
| 58 | Plasma retinol-binding protein precursor | P02753 | 22995.26 | 9.33E−14 | 0549; 0560; 0574; |
| | | | | | 0567; 0552; 0556; |
| | | | | | 0535; 0549; 0571 |
| 59 | Centrosomal protein of 110 kDa | O43303 | 113370.6 | 1.18E−03 | 0541; 0556; 0574; |
| | | | | | 0567; 0552; 0578; |
| | | | | | 0560 |
| 60 | Creatine kinase B-type | P12277 | 42617.37 | 2.00E−14 | 0560; 0578; 0552; |
| | | | | | 0556; 0574; 0571 |
| 61 | Hypothetical protein | Q569I7 | 20655.36 | 3.33E−13 | 0535; 0552; 0578; |
| | | | | | 0556; 0571; 0574 |
| 62 | Polymeric-immunoglobulin receptor precursor | P01833 | 83261.7 | 8.66E−14 | 0571; 0552; 0578; |
| | | | | | 0574 |
| 63 | Epididymal secretory protein E1 precursor | P61916 | 16559.49 | 3.80E−11 | 0578; 0574; 0560; |
| | | | | | 0567; 0556; 0552; |
| | | | | | 0571; 0535; 0549 |
| 64 | Tumor-associated hydroquinone oxidase | Q16206 | 70037.12 | 1.03E−03 | 0578; 0552; 0574; |
| | | | | | 0567; 0549; 0560; |
| | | | | | 0556; 0578; 0535 |
| 65 | Hypothetical protein (Fragment) | Q96BI9 | 12421.42 | 2.11E−03 | 0567; 0578; 0560; |
| | | | | | 0541; 0574; 0549; |
| | | | | | 0552; 0556 |
| 66 | Hypothetical protein DKFZp686I04196 (Fragment) | Q6N093 | 46031.66 | 1.19E−10 | 0535; 0578; 0552; |
| | | | | | 0556; 0567; 0571 |
| 67 | Type III iodothyronine deiodinas | P55073 | 31383.73 | 1.23E−05 | 0571; 0541; 0567; |
| | | | | | 0560; 0578 |
| 68 | Beta-globin gene from a thalassemia patient | Q14473 | 18918.59 | 1.32E−13 | 0552; 0560; 0578; |
| | | | | | 0571 |
| 69 | Hypothetical protein FLJ38687 | Q8N8Z1 | 16623.52 | 1.19E−03 | 0541; 0574; 0535; |
| | | | | | 0571; 0556; 0578; |
| | | | | | 0552; 0560 |
| 70 | Complement factor I precursor | P05156 | 65676.66 | 5.73E−09 | 0552; 0541; 0574; |
| | | | | | 0571; 0567; 0556; |
| | | | | | 0560 |
| 71 | Hypothetical protein | Q6PHR9 | 9188.304 | 8.51E−04 | 0574; 0552; 0535; |
| | | | | | 0571; 0578; 0556; |
| | | | | | 0541 |
| 72 | Alpha-enolase | P06733 | 47008.33 | 1.00E−30 | 0574; 0560; 0571; |
| | | | | | 0578; 0567; 0549 |
| 73 | DEAH (Asp-Glu-Ala-His) box polypeptide 29 | Q7Z478 | 155178.3 | 2.50E−03 | 0578; 0552; 0567; |
| | | | | | 0556; 0571; 0560 |
| 74 | PH domain containing protein | Q8TD55 | 53317.19 | 2.16E−04 | 0560; 0556; 0541; |
| | | | | | 0574; 0571; 0567 |
| 75 | Phosphoglycerate kinase 1 | P00558 | 44455.11 | 4.93E−12 | 0560; 0578; 0556; |
| | | | | | 0541 |
| 76 | GTP:AMP phosphotransferase mitochondrial | Q9UIJ7 | 25418.51 | 1.22E−03 | 0578; 0556; 0567; |
| | | | | | 0552; 0571; 0560; |
| | | | | | 0541 |
| 77 | Hypothetical protein FLJ42076 | Q6ZVU4 | 21036.72 | 4.61E−04 | 0535; 0567; 0571; |
| | | | | | 0560; 0552; 0574 |
| 78 | Antithrombin III variant | Q7KZ97 | 52657.96 | 8.82E−11 | 0556; 0552; 0541; |
| | | | | | 0535; 0574; 0560 |
| 79 | Hypothetical protein FLJ32091 | Q96MP4 | 81413.94 | 1.13E−03 | 0578; 0549; 0552; |
| | | | | | 0535; 0574; 0541 |
| 80 | WUGSC:H_2G3A.1 protein | Q99993 | 60051.64 | 1.27E−04 | 0552; 0574; 0571; |
| | | | | | 0567; 0560; 0541 |
| 81 | Triosephosphate isomerase | P60174 | 26521.7 | 2.90E−10 | 0560; 0578; 0556 |
| 82 | Phosphatidylethanolamine-binding protein | P30086 | 20912.63 | 5.71E−10 | 0578; 0560; 0552 |
| 83 | Arginyl-tRNA synthetase | P54136 | 75330.97 | 1.61E−03 | 0541; 0578; 0556; |
| | | | | | 0574; 0560; 0567; |
| | | | | | 0571; 0549 |
| 84 | Tripeptidyl-peptidase I precursor | O14773 | 61190.67 | 1.00E−30 | 0541; 0567; 0552; |
| | | | | | 0560; 0556; 0571; |
| | | | | | 0574 |
| 85 | FNBP1 protein (Fragment) | Q6P658 | 40759.38 | 6.47E−04 | 0535; 0556; 0567; |
| | | | | | 0549; 0578; 0574; |
| | | | | | 0560 |
| 86 | Hypothetical protein FLJ25756 | Q8N7D8 | 47133.98 | 2.79E−04 | 0560; 0556; 0535; |
| | | | | | 0541; 0578; 0571; |
| | | | | | 0567 |
| 87 | Alpha-1B-glycoprotein precursor | P04217 | 54238.7 | 6.66E−15 | 0552; 0549; 0535; |
| | | | | | 0541; 0574; 0567 |
| 88 | Kininogen-1 precursor | P01042 | 71900.1 | 3.68E−06 | 0541; 0535; 0552; |
| | | | | | 0574; 0556; 0567 |
| 89 | Amyloid-like protein 1 precursor | P51693 | 72131.33 | 7.51E−08 | 0552; 0560; 0567; |
| | | | | | 0574; 0541; 0571 |
| 90 | Zinc-alpha-2-glycoprotein precursor | P25311 | 33850.91 | 4.58E−12 | 0552; 0571; 0541; |
| | | | | | 0574; 0560 |
| 91 | Apolipoprotein A-IV precursor | P06727 | 45343.52 | 2.91E−13 | 0535; 0541; 0560; |
| | | | | | 0556; 0552 |
| 92 | Hypothetical protein FLJ45254 | Q6ZSR8 | 115180.5 | 7.91E−04 | 0556; 0535; 0567; |
| | | | | | 0578; 0560 |
| 93 | SERPINC1 protein | Q8TCE1 | 29073.79 | 8.37E−06 | 0556; 0549; 0541; |
| | | | | | 0535; 0574 |
| 94 | Pyruvate kinase, isozymes M1/M2 | P14618 | 57769.12 | 1.57E−11 | 0560; 0578; 0556 |
| 95 | Fibrinogen gamma chain precursor | P02679 | 51478.88 | 1.55E−11 | 0552; 0535 |
| 96 | MAx-like protein X | Q9UH92 | 33279.74 | 2.09E−03 | 0556; 0535; 0549; |
| | | | | | 0578; 0574; 0552; |
| | | | | | 0541 |
| 97 | Amyloid beta A4 protein precursor | P05067 | 86888.16 | 6.25E−08 | 0541; 0567; 0571; |
| | | | | | 0574; 0549 |
| 98 | Ribonuclease pancreatic precursor | P07998 | 17632.68 | 7.40E−12 | 0567; 0560; 0552; |
| | | | | | 0541; 0574 |
| 99 | Ectonucleotide pyrophosphatase/phosphodiesterase 2 | Q13822 | 98939.61 | 1.11E−15 | 0552; 0567; 0578; |
| | | | | | 0571; 0574 |
| 100 | Novel protein (Fragment) | Q5T2T0 | 35987.71 | 3.87E−03 | 0571; 0541; 0578; |
| | | | | | 0549; 0567 |
| 101 | Hypothetical protein FLJ22582 | Q9H661 | 25812.05 | 1.82E−03 | 0560; 0578; 0571; |
| | | | | | 0556; 0567 |
| 102 | Collagen alpha 1(II) chain precursor | P02458 | 134410.6 | 7.90E−06 | 0556; 0578; 0552; |
| | | | | | 0574 |
| 103 | CRBS_HUMAN (P22914) Beta crystallin S (Gamma crystallin S) | P22914 | 20861.95 | 1.11E−14 | 0549; 0574; 0578 |
| 104 | ARRS_HUMAN (P10523) S-arrestin (Retinal S-antigen) (48 kDa | P10523 | 45063.49 | 1.07E−10 | 0578; 0560; 0574 |
| | protein) (S-AG) (Rod photoreceptor arres | | | | |
| 105 | H4_HUMAN (P62805) Histone H4 | P62805 | 11229.34 | 1.03E−09 | 0560; 0578; 0574 |
| 106 | Q4ZG55 (Q4ZG55) Hypothetical protein GREB1 | Q4ZG55 | 216328.6 | 4.35E−04 | 0560; 0552; 0574 |
| 107 | Q53F97 (Q53F97) Alpha 2 globin variant (Fragment) | Q53F97 | 15270.94 | 2.22E−16 | 0552; 0560 |
| 108 | FIBB_HUMAN (P02675) Fibrinogen beta chain precursor [Contains: | P02675 | 55892.23 | 1.90E−12 | 0552; 0535 |
| | Fibrinopeptide B] | | | | |
| 109 | ENOG_HUMAN (P09104) Gamma-enolase (EC 4.2.1.11) (2-phospho- | P09104 | 47108.02 | 6.67E−11 | 0578; 0560 |
| | D-glycerate hydro-lyase) (Neural enolase | | | | |
| 110 | O43308 (O43308) KIAA0425 protein (Fragment) | O43308 | 141056.5 | 2.24E−04 | 0541; 0578; 0549; |
| | | | | | 0560; 0556; 0552; |
| | | | | | 0574 |
| 111 | NU214_HUMAN (P35658) Nuclear pore complex protein Nup214 | P35658 | 213632.8 | 4.87E−05 | 0549; 0578; 0541; |
| | (Nucleoporin Nup214) (214 kDa nucleoporin) | | | | 0556; 0552; 0535; |
| | | | | | 0571 |
| 112 | Q86UQ8 (Q86UQ8) Transcription factor NF-E4 | Q86UQ8 | 19006.73 | 1.03E−04 | 0574; 0549; 0541; |
| | | | | | 0535; 0560; 0552; |
| | | | | | 0556 |
| 113 | Q8N8R5 (Q8N8R5) Hypothetical protein FLJ38973 | Q8N8R5 | 43420.63 | 3.35E−03 | 0567; 0560; 0571; |
| | | | | | 0574; 0549; 0578; |
| | | | | | 0556 |
| 114 | Q58A63 (Q58A63) BNIP2 motif containing molecule at the carboxyl | Q58A63 | 298881.4 | 4.28E−04 | 0571; 0556; 0552; |
| | tarminal region 1 | | | | 0560; 0574; 0535 |
| 115 | PCLO_HUMAN (Q9Y6V0) Piccolo protein (Aczonin) | Q9Y6V0 | 566313.1 | 6.19E−06 | 0567; 0541; 0571; |
| | | | | | 0535; 0552; 0556 |
| 116 | B3GN6_HUMAN (O43505) N-acetyllactosaminide beta-1,3-N- | Q43505 | 47088.94 | 1.31E−08 | 0567; 0574; 0541; |
| | acetylglucosaminyltransferase (EC 2.4.1.149) | | | | 0571; 0552 |
| 117 | CSTN1_HUMAN (O94985) Calsyntenin-1 precursor | O94985 | 109723.7 | 2.63E−08 | 0578; 0574; 0552; |
| | | | | | 0567; 0541 |
| 118 | Q7Z4H7 (Q7Z4H7) Hypothetical protein | Q7Z4H7 | 108566.8 | 9.15E−04 | 0571; 0560; 0578; |
| | | | | | 0556; 0567 |
| 119 | OSR1_HUMAN (Q9BXW6) Oxysterol binding protein-related protein 1 | Q9BXW6 | 108401.1 | 5.02E−03 | 0560; 0535; 0574; |
| | (OSBP-related protein 1) (ORP-1) | | | | 0552; 0556 |
| 120 | GSCR1_HUMAN (Q9NZM4) Glioma tumor suppressor candidate | Q9NZM4 | 152898.4 | 2.21E−04 | 0560; 0571; 0541; |
| | region gene 1 protein | | | | 0535; 0578 |
| 121 | COPG2_HUMAN (Q9UBF2) Coatomer gamma-2 subunit (Gamma-2 | Q9UBF2 | 97559.69 | 4.15E−04 | 0535; 0571; 0549; |
| | coat protein) (Gamma-2 COP) | | | | 0578; 0567 |
| 122 | ACTA_HUMAN (P62736) Actin, aortic smooth muscle (Alpha-actin-2) | P62736 | 41981.82 | 1.58E−10 | 0552; 0560; 0574; |
| | | | | | 0578 |
| 123 | SAA_HUMAN (P02735) Serum amyloid A protein precursor (SAA) | P02735 | 13523.52 | 9.99E−15 | 0552; 0535; 0556 |
| | [Contains: Amyloid protein A (Amyloid f [symbol] | | | | |
| 124 | CRYAA_HUMAN (P02489) Alpha crystallin A chain (Heat-shock | P02489 | 19896.9 | 8.88E−15 | 0549; 0574 |
| | porotein beta-4) (HspB4) | | | | |
| 125 | ACADV_HUMAN (P49748) Acyl-CoA dehydrogenase, very-long-chain | P49748 | 70345.54 | 8.15E−04 | 0560; 0541; 0571; |
| | specific, mitochondrial precursor (EC | | | | 0574; 0567; 0556 |
| 126 | A2GL_HUMAN (P02750) Leucine-rich alpha-2-glycoprotein precursor | P02750 | 38154.13 | 3.33E−15 | 0552; 0549; 0535; |
| | (LRG) | | | | 0541; 0571 |
| 127 | SPAG6_HUMAN (O75602) Sperm-associated antigen 6 (PF16 protein | O75602 | 55441.33 | 1.26E−06 | 0578; 0552; 0571; |
| | homolog) (Sperm flagellar protein) (R | | | | 0574; 0560 |
| 128 | BTD_HUMAN (P43251) Biotinidase precursor (EC 3.5.1.12) | P43251 | 58875.13 | 1.65E−07 | 0567; 0571; 0574; |
| | | | | | 0578; 0552 |
| 129 | Q8WVN8 (Q8WVN8) Hypothetical protein LOC92912 (Hypothetical | Q8WVN8 | 42791.48 | 7.75E−04 | 0549; 0541; 0556; |
| | protein DKFZp762C143) | | | | 0552; 0571 |
| 130 | SPRL1_HUMAN (Q14515) SPARC-like protein 1 precursor (High | Q14515 | 75169.27 | 5.28E−08 | 0541; 0578; 0571; |
| | endothelial venule protein) (Hevin) (MAST | | | | 0552 |
| 131 | Q86UZ9 (Q86UZ9) Signal peptide, CUB domain, EGF-like 3 | Q86UZ9 | 109092.9 | 2.40E−04 | 0541; 0578; 0567; |
| | | | | | 0549 |
| 132 | MYOC_HUMAN (Q99972) Myocilin precursor (Trabecular meshwork- | Q99972 | 56936.61 | 2.34E−07 | 0578; 0567; 0560; |
| | induced glucocorticoid response protein | | | | 0541 |
| 133 | Q9H6T0 (Q9H6T0) Hypothetical protein FLJ21918 | Q9H6T0 | 78351.28 | 2.37E−03 | 0574; 0560; 0571; |
| | | | | | 0567 |
| 134 | Q9Y6R7 (Q9Y6R7) Fc gamma BP (Fragment) | Q9Y6R7 | 301606.7 | 5.53E−08 | 0556; 0541; 0574 |
| 135 | MT1E_HUMAN (P04732) Metallothionein-1E (MT-1E) | P04732 | 6009.202 | 1.00E−08 | 0560; 0571; 0552 |
| | (Metallothionein-IE) (MT-IE) | | | | |
| 136 | CRYAB_HUMAN (P02511) Alpha crystallin B chain (Alpha(B)- | P02511 | 20146.43 | 4.88E−12 | 0549; 0574 |
| | crystallin) (Rosenthal fiber component) (He | | | | |
| 137 | Q59EM9 (Q59EM9) Ubiquitin C variant (Fragment) | Q59EM9 | 147245 | 3.85E−06 | 0578; 0560 |
| 138 | SVIL_HUMAN (O95425) Supervillin (Archvillin) (p205/p250) | O95425 | 247550.6 | 9.46E−04 | 0549; 0535; 0571; |
| | | | | | 0560; 0552 |
| 139 | IBP6_HUMAN (P24592) Insulin-like growth factor binding protein 6 | P24592 | 25306.17 | 1.11E−06 | 0567; 0578; 0574; |
| | precursor (IGFBP-6) (IBP-6) (IGF-b | | | | 0571; 0560 |
| 140 | K0179_HUMAN (Q14684) Protein KIAA0179 | Q14684 | 84359.22 | 6.11E−04 | 0571; 0574; 0560; |
| | | | | | 0578; 0552 |
| 141 | Q9BSG5 (Q9BSG5) Retbindin | Q9BSG5 | 28145.91 | 4.76E−07 | 0541; 0574; 0567; |
| | | | | | 0578; 0552 |
| 142 | LUM_HUMAN (P51884) Lumican precursor (Keratan sulfate | P51884 | 38404.8 | 2.97E−06 | 0535; 0556; 0578; |
| | proteoglycan lumican) (KSPG lumican) | | | | 0552 |
| 143 | CO9A2_HUMAN (Q14055) Collagen alpha 2(IX) chain precursor | Q14055 | 65090.96 | 3.06E−06 | 0578; 0567; 0574; |
| | | | | | 0541 |
| 144 | GRB14_HUMAN (Q14449) Growth factor receptor-bound protein 14 | Q14449 | 60949.65 | 2.36E−03 | 0556; 0574; 0541; |
| | (GRB14 adapter protein) | | | | 0535 |
| 145 | Q5VV79 (Q5VV79) SNF2 histone linker PHD RING helicase | Q5VV79 | 192955.8 | 1.00E−03 | 0560; 0571; 0567; |
| | | | | | 0552 |
| 146 | Q8NDD1 (Q8NDD1) Hypothetical protein DKFZp547B1713 | Q8NDD1 | 32732.49 | 2.07E−03 | 0560; 0535; 0574; |
| | | | | | 0549 |
| 147 | CPSF3_HUMAN (Q9UKF6) Cleavage and polyadenylation specificity | Q9UKF6 | 77436.22 | 3.07E−04 | 0578; 0552; 0574; |
| | factor, 73 kDa subunit (CPSF 73 kDa s | | | | 0541 |
| 148 | H2AA_HUMAN (P28001) Histone H2A.a (H2A/a) (H2A.2) | P28001 | 13995.91 | 3.39E−11 | 0560; 0574; 0552 |
| 149 | PROL4_HUMAN (Q16378) Proline-rich protein 4 precursor (Lacrimal | Q16378 | 15087.6 | 3.75E−11 | 0549; 0552; 0574 |
| | proline-rich protein) (Nasopharynge | | | | |
| 150 | LACRT_HUMAN (Q9GZZ8) Extracellular glycoprotein lacritin | Q9GZZ8 | 14237.34 | 2.99E−04 | 0552; 0560; 0574 |
| | precursor | | | | |
| 151 | CELR1_HUMAN (Q9NYQ6) Cadherin EGF LAG seven-pass G-type | Q9NYQ6 | 329276.7 | 2.03E−04 | 0556; 0578; 0535 |
| | receptor 1 precursor (Flamingo homolog 2) ( | | | | |
| 152 | CRBB1_HUMAN (P53674) Beta crystallin B1 | P53674 | 27874.85 | 4.88E−14 | 0549; 0574 |
| 153 | CRBA4_HUMAN (P53673) Beta crystallin A4 (Beta-A4-crystallin) | P53673 | 22228.56 | 1.00E−30 | 0549; 0574 |
| 154 | VIME_HUMAN (P08670) Vimentin | P08670 | 53488.13 | 4.45E−09 | 0560; 0574 |
| 155 | 1433G_HUMAN (P61981) 14-3-3 protein gamma (Protein kinase C | P61981 | 28153.87 | 1.62E−11 | 0578; 0560 |
| | inhibitor protein 1) (KCIP-1) | | | | |
| 156 | Q9P2D3 (Q9P2D3) KIAA1414 protein (Fragment) | Q9P2D3 | 171852.9 | 5.07E−03 | 0535; 0560; 0556; |
| | | | | | 0578; 0552 |
| 157 | Q86VP1 (Q86VP1) Tax1 (Human T-cell leukemia virus type I) binding | Q86VP1 | 90819.88 | 5.39E−04 | 0549; 0556; 0574; |
| | protein 1 | | | | 0578 |
| 158 | PLMN_HUMAN (P00747) Plasminogen precursor (EC 3.4.21.7) | P00747 | 90510.23 | 2.03E−05 | 0552; 0541; 0535; |
| | [Contains: Plasmin heavy chain A; Activatio | | | | 0556 |
| 159 | PDE6A_HUMAN (P16499) Rod cGMP-specific 3′,5′-cyclic | P16499 | 99438.76 | 1.80E−03 | 0567; 0571; 0556; |
| | phosphodiesterase alpha-subunit (EC 3.1.4.35) | | | | 0574 |
| 160 | RHG25_HUMAN (P42331) Rho-GTPase-activating protein 25 | P42331 | 72385.19 | 1.85E−03 | 0535; 0556; 0560; |
| | | | | | 0541 |
| 161 | RBP2_HUMAN (P49792) Ran-binding protein 2 (RanBP2) (Nuclear | P49792 | 357991.4 | 3.05E−04 | 0567; 0552; 0571; |
| | pore complex protein Nup358) (Nucleopor | | | | 0560 |
| 162 | Q6P988 (Q6P988) Hypothetical protein LOC147111 | Q6P988 | 48570.99 | 3.74E−04 | 0571; 0541; 0556; |
| | | | | | 0567 |
| 163 | Q6ZTG8 (Q6ZTG8) Hypothetical protein FLJ44670 | Q6ZTG8 | 115755 | 1.14E−04 | 0560; 0578; 0556; |
| | | | | | 0574 |
| 164 | Q7Z5K1 (Q7Z5K1) Leukocyte-derived arginine aminopeptidase long | Q7Z5K1 | 110377.1 | 1.04E−03 | 0571; 0567; 0578; |
| | form variant | | | | 0574 |
| 165 | Q8N401 (Q8N401) KIAA1632 protein | Q8N401 | 52340.19 | 5.40E−04 | 0574; 0556; 0552; |
| | | | | | 0541 |
| 166 | NR4A3_HUMAN (Q92570) Orphan nuclear receptor NR4A3 (Nuclear | Q92570 | 68156.35 | 5.88E−04 | 0535; 0578; 0571; |
| | hormone receptor NOR-1) (Neuron-derived | | | | 0556 |
| 167 | RT17_HUMAN (Q9Y2R5) 28S ribosomal protein S17, mitochondrial | Q9Y2R5 | 14492.93 | 1.18E−03 | 0535; 0552; 0578; |
| | precursor (S17mt) (MRP-S17) | | | | 0549 |
| 168 | FA5_HUMAN (P12259) Coagulation factor V precursor (Activated | P12259 | 251511.8 | 1.05E−04 | 0552; 0571; 0556 |
| | protein C cofactor) [Contains: Coagula | | | | |
| 169 | TBA1_HUMAN (P68366) Tubulin alpha-1 chain (Alpha-tubulin 1) | P68366 | 49892.41 | 3.40E−09 | 0560; 0574; 0578 |
| | (Testis-specific alpha-tubulin) (Tubuli | | | | |
| 170 | Q5T714 (Q5T714) Ash1 (Absent, small, or homeotic)-like (Drosophila) | Q5T714 | 332580 | 2.25E−05 | 0556; 0560; 0535 |
| 171 | LDH6A_HUMAN (Q6ZMR3) L-lactate dehydrogenase A-like 6A (EC | Q6ZMR3 | 36484.2 | 1.30E−09 | 0578; 0560; 0556 |
| | 1.1.1.27) | | | | |
| 172 | Q86TT2 (Q86TT2) Full-length cDNA clone CS0DI019YF20 of | Q86TT2 | 39100.3 | 1.08E−10 | 0578; 0556; 0574 |
| | Placenta of Homo sapiens (human) (Fragment) | | | | |
| 173 | Q86YR7 (Q86YR7) Rho family guanine-nucleotide exchange factor | Q86YR7 | 117435.9 | 1.92E−03 | 0541; 0571; 0549 |
| 174 | Q8N7W7 (Q8N7W7) Hypothetical protein FLJ40259 | Q8N7W7 | 66086.88 | 1.46E−03 | 0552; 0541; 0574 |
| 175 | RLP24_HUMAN (Q9UHA3) Probable ribosome biogenesis protein | Q9UHA3 | 19608.34 | 6.29E−04 | 0567; 0578; 0574 |
| | RLP24 (Ribosomal protein L24-like) | | | | |
| 176 | IGJ_HUMAN (P01591) Immunoglobulin J chain | P01591 | 15584.55 | 1.13E−06 | 0571; 0552 |
| 177 | CRBA1_HUMAN (P05813) Beta crystallin A3 [Contains: Beta | P05813 | 25133.82 | 3.33E−15 | 0549 |
| | crystallin A1] | | | | |
| 178 | CRGC_HUMAN (P07315) Gamma crystallin C (Gamma crystallin 2-1) | P07315 | 20733.98 | 8.54E−12 | 0549 |
| | (Gamma crystallin 3) | | | | |
| 179 | CRGD_HUMAN (P07320) Gamma crystallin D (Gamma crystallin 4) | P07320 | 20593.8 | 1.11E−15 | 0549 |
| 180 | FABPL_HUMAN (P07148) Fatty acid-binding protein, liver (L-FABP) | P07148 | 14199.41 | 3.54E−06 | 0560 |
| 181 | THRB_HUMAN (P00734) Prothrombin precursor (EC 3.4.21.5) | P00734 | 69992.2 | 5.83E−10 | 0549; 0535; 0541; |
| | (Coagulation factor II) [Contains: Activati | | | | 0552 |
| 182 | A2AP_HUMAN (P08697) Alpha-2-antiplasmin precursor (Alpha-2- | P08697 | 54531.18 | 1.53E−11 | 0556; 0574; 0541; |
| | plasmin inhibitor) (Alpha-2-PI) (Alpha-2 | | | | 0552 |
| 183 | LG3BP_HUMAN (Q08380) Galectin-3 binding protein precursor | Q08380 | 65289.4 | 4.45E−09 | 0560; 0541; 0567; |
| | (Lectin galactoside-binding soluble 3 bin | | | | 0578 |
| 184 | TIC1_HUMAN (Q08629) Testican-1 precursor (SPOCK protein) | Q08629 | 49092.49 | 7.92E−06 | 0567; 0541; 0574; |
| | | | | | 0560 |
| 185 | GOGA4_HUMAN (Q13439) Golgi autoantigen, golgin subfamily A | Q13439 | 260978.6 | 5.90E−04 | 0578; 0567; 0556; |
| | member 4 (Trans-Golgi p230) (256 kDa gol | | | | 0552 |
| 186 | UBP26_HUMAN (Q9BXU7) Ubiquitin carboxyl-terminal hydrolase 26 | Q9BXU7 | 103981.2 | 1.44E−04 | 0567; 0571; 0556; |
| | (EC 3.1.2.15) (Ubiquitin thiolesteras | | | | 0535 |
| 187 | MYG_HUMAN (P02144) Myoglobin | P02144 | 17041.91 | 5.67E−11 | 0535; 0578; 0560 |
| 188 | CH3L1_HUMAN (P36222) Chitinase-3-like protein 1 precursor | P36222 | 42586.39 | 1.70E−06 | 0560; 0556; 0574 |
| | (Cartilage glycoprotein 39) (GP-39) (39 k | | | | |
| 189 | SEM7A_HUMAN (O75326) Semaphorin-7A precursor (Semaphorin L) | O75326 | 74776.28 | 3.11E−07 | 0574; 0541; 0571 |
| | (Sema L) (Semaphorin K1) (Sema K1) (Joh | | | | |
| 190 | NCTR1_HUMAN (O76036) Natural cytotoxicity triggering receptor 1 | O76036 | 34458.41 | 1.63E−03 | 0578; 0560; 0535 |
| | precursor (Natural killer cell p46- | | | | |
| 191 | FETUA_HUMAN (P02765) Alpha-2-HS-glycoprotein precursor | P02765 | 39299.73 | 1.82E−06 | 0535; 0541; 0571 |
| | (Fetuin-A) (Alpha-2-Z-globulin) (Ba-alpha-2- | | | | |
| 192 | HRG_HUMAN (P04196) Histidine-rich glycoprotein precursor | P04196 | 59540.94 | 3.64E−06 | 0549; 0535; 0556 |
| | (Histidine-proline-rich glycoprotein) (HPR | | | | |
| 193 | GSTP1_HUMAN (P09211) Glutathione S-transferase P (EC 2.5.1.18) | P09211 | 23209.98 | 4.06E−11 | 0578; 0552; 0571 |
| | (GST class-pi) (GSTP1-1) | | | | |
| 194 | CDK6_HUMAN (Q00534) Cell division protein kinase 6 (EC 2.7.1.37) | Q00534 | 36915.05 | 2.90E−03 | 0552; 0578; 0571 |
| | (Serine/threonine-protein kinase P | | | | |
| 195 | VAS1_HUMAN (Q15904) Vacuolar ATP synthase subunit S1 | Q15904 | 51992.65 | 2.50E−10 | 0574; 0541; 0578 |
| | precursor (EC 3.6.3.14) (V-ATPase S1 subunit) | | | | |
| 196 | IBP7_HUMAN (Q16270) Insulin-like growth factor binding protein 7 | Q16270 | 29111.45 | 3.67E−06 | 0560; 0549; 0578 |
| | precursor (IGFBP-7) (IBP-7) (IGF-b | | | | |
| 197 | Q5CZ94 (Q5CZ94) Hypothetical protein DKFZp781M0386 | Q5CZ94 | 24986.3 | 7.58E−06 | 0535; 0571; 0567 |
| 198 | Q6ZMC8 (Q6ZMC8) Hypothetical protein FLJ23997 | Q6ZMC8 | 49902.74 | 5.55E−04 | 0556; 0567; 0560 |
| 199 | Q6ZRY1 (Q6ZRY1) Hypothetical protein FLJ45988 | Q6ZRY1 | 40475.38 | 4.15E−03 | 0535; 0552; 0578 |
| 200 | Q8N9C3 (Q8N9C3) Hypothetical protein FLJ37770 | Q8N9C3 | 24197.5 | 1.73E−03 | 0571; 0556; 0535 |
| 201 | Q8NHQ3 (Q8NHQ3) RBBP8 protein | Q8NHQ3 | 102594.6 | 3.62E−04 | 0567; 0560 |
| 202 | SPON1_HUMAN (Q9HCB6) Spondin-1 precursor (F-spondin) | Q9HCB6 | 90913.51 | 8.16E−11 | 0541; 0574 |
| | (Vascular smooth muscle cell growth promoting | | | | |
| 203 | Q9UGL1 (Q9UGL1) RB-binding protein | Q9UGL1 | 189996.1 | 4.41E−03 | 0549; 0535 |
| 204 | G3P1_HUMAN (P00354) Glyceraldehyde-3-phosphate | P00354 | 35853.31 | 1.56E−07 | 0578; 0552 |
| | dehydrogenase, muscle (EC 1.2.1.12) (GAPDH) | | | | |
| 205 | 1433B_HUMAN (P31946) 14-3-3 protein beta/alpha (Protein kinase C | P31946 | 27933.79 | 4.35E−11 | 0578; 0560 |
| | inhibitor protein 1) (KCIP-1) (Pro | | | | |
| 206 | DEF1_HUMAN (P59665) Neutrophil defensin 1 precursor (HNP-1) | P59665 | 10194.18 | 7.54E−06 | 0578; 0552 |
| | (HP-1) (HP1) (Defensin, alpha 1) [Conta | | | | |
| 207 | TBA2_HUMAN (Q13748) Tubulin alpha-2 chain (Alpha-tubulin 2) | Q13748 | 49927.65 | 1.13E−10 | 0560; 0578 |
| 208 | Q6MZX7 (Q6MZX7) Hypothetical protein DKFZp686M24218 | Q6MZX7 | 52387.13 | 3.84E−09 | 0535; 0567 |
| 209 | Q6PI81 (Q6PI81) IGHM protein | Q6PI81 | 52633.13 | 6.49E−08 | 0571; 0556 |
| 210 | Q7Z5B4 (Q7Z5B4) RIC3 | Q7Z5B4 | 41066.16 | 3.61E−03 | 0541; 0571 |
| 211 | Q7Z745 (Q7Z745) Hypothetical protein FLJ40243 | Q7Z745 | 180566.4 | 1.42E−03 | 0578; 0556 |
| 212 | PARK7_HUMAN (Q99497) DJ-1 protein (Oncogene DJ1) | Q99497 | 19878.49 | 4.50E−13 | 0560; 0549 |
| 213 | BFSP1_HUMAN (Q12934) Filensin (Beaded filament structural protein | Q12934 | 74499.29 | 1.47E−10 | 0549 |
| | 1) (Lens fiber cell beaded-filame | | | | |
| 214 | BFSP2_HUMAN (Q13515) Phakinin (Beaded filament structural | Q13515 | 45851.28 | 1.45E−11 | 0549 |
| | protein 2) (Lens fiber cell beaded filame | | | | |
| 215 | K1C9_HUMAN (P35527) Keratin, type I cytoskeletal 9 (Cytokeratin-9) | P35527 | 62091.76 | 1.11E−16 | 0574 |
| | (CK-9) (Keratin-9) (K9) | | | | |
| 216 | Q6PJ95 (Q6PJ95) IGHG1 protein | Q6PJ95 | 60064.1 | 1.16E−05 | 0556 |
| 217 | Q86VJ1 (Q86VJ1) E3 ligase for inhibin receptor | Q86VJ1 | 289427.4 | 1.25E−03 | 0571 |
| 218 | SELH_HUMAN (Q8IZQ5) Selenoprotein H | Q8IZQ5 | 13398.19 | 8.21E−03 | 0552 |
| 219 | CSPG2_HUMAN (P13611) Versican core protein precursor (Large | P13611 | 372589 | 1.50E−11 | 0571; 0574; 0567 |
| | fibroblast proteoglycan) (Chondroitin s | | | | |
| 220 | PDZK3_HUMAN (O15018) PDZ domain containing protein 3 (PDZ | O15018 | 301423.4 | 1.38E−04 | 0560; 0541; 0578 |
| | domain containing protein 2) (Activated i | | | | |
| 221 | XLRS1_HUMAN (O15537) Retinoschisin precursor (X-linked juvenile | O15537 | 25575.54 | 2.10E−09 | 0541; 0567; 0552 |
| | retinoschisis protein) | | | | |
| 222 | DEPD5_HUMAN (O75140) DEP domain containing protein 5 | O75140 | 177796 | 2.96E−03 | 0574; 0567; 0552 |
| 223 | O95978 (O95978) VH1 protein precursor (Fragment) | O95978 | 17292.5 | 3.63E−08 | 0552; 0556; 0560 |
| 224 | HV3S_HUMAN (P01780) Ig heavy chain V-III region JON | P01780 | 12555.34 | 9.51E−07 | 0556; 0535; 0578 |
| 225 | CFAH_HUMAN (P08603) Complement factor H precursor (H factor 1) | P08603 | 139033.4 | 2.48E−06 | 0552; 0535; 0541 |
| 226 | NEC2_HUMAN (P16519) Neuroendocrine convertase 2 precursor (EC | P16519 | 70520.62 | 1.43E−08 | 0571; 0560; 0567 |
| | 3.4.21.94) (NEC 2) (PC2) (Prohormone | | | | |
| 227 | 3BP2_HUMAN (P78314) SH3 domain-binding protein 2 (3BP-2) | P78314 | 62204.26 | 1.58E−03 | 0578; 0567; 0552 |
| 228 | TF3C1_HUMAN (Q12789) General transcription factor 3C polypeptide | Q12789 | 238143.4 | 5.36E−04 | 0574; 0578; 0552 |
| | 1 (Transcription factor IIIC-alpha | | | | |
| 229 | NAF1_HUMAN (Q15025) Nef-associated factor 1 (Naf1) (TNFAIP3- | Q15025 | 71819.7 | 2.15E−04 | 0567; 0560; 0578 |
| | interacting protein 1) (HIV-1 Nef inter | | | | |
| 230 | Q4VBZ9 (Q4VBZ9) MORC family CW-type zinc finger 3 | Q4VBZ9 | 106973.1 | 2.29E−03 | 0578; 0541; 0560 |
| 231 | Q5BLQ2 (Q5BLQ2) Mucin | Q5BLQ2 | 289658.2 | 4.49E−03 | 0567; 0578; 0541 |
| 232 | Q5FWF9 (Q5FWF9) Hypothetical protein | Q5FWF9 | 24779.14 | 1.83E−04 | 0535; 0578; 0567 |
| 233 | Q5HY96 (Q5HY96) Hypothetical protein DKFZp686C0394 | Q5HY96 | 173231.1 | 2.82E−03 | 0552; 0574; 0578 |
| 234 | Q5JPH6 (Q5JPH6) Hypothetical protein DKFZp686C2322 | Q5JPH6 | 58621.73 | 7.27E−04 | 0560; 0578; 0574 |
| 235 | Q5VVM6 (Q5VVM6) Novel protein | Q5VVM6 | 91277.53 | 7.22E−04 | 0552; 0574; 0571 |
| 236 | Q5VWH1 (Q5VWH1) Tumor necrosis factor (Ligand) superfamily, | Q5VWH1 | 20099.21 | 1.82E−03 | 0556; 0541; 0552 |
| | member 15 | | | | |
| 237 | Q6ZND7 (Q6ZND7) Hypothetical protein FLJ16192 | Q6ZND7 | 58888.55 | 1.49E−03 | 0567; 0574; 0541 |
| 238 | Q6ZRF7 (Q6ZRF7) Hypothetical protein FLJ46385 | Q6ZRF7 | 15503.12 | 3.05E−03 | 0535; 0574; 0560 |
| 239 | Q70EX7 (Q70EX7) Sodium-dependent organic anion transporter | Q70EX7 | 41231.12 | 1.26E−03 | 0541; 0567; 0574 |
| 240 | Q8N264 (Q8N264) Hypothetical protein FLJ33877 | Q8N264 | 84143.33 | 2.16E−03 | 0541; 0560; 0552 |
| 241 | ASM3B_HUMAN (Q92485) Acid sphingomyelinase-like | Q92485 | 50781.24 | 3.66E−03 | 0541; 0567; 0535 |
| | phosphodiesterase 3b precursor (EC 3.1.4.—) (ASM-li | | | | |
| 242 | Q96C45 (Q96C45) ULK4 protein | Q96C45 | 64932.5 | 2.43E−04 | 0549; 0560; 0535 |
| 243 | CYH2_HUMAN (Q99418) Cytohesin-2 (ARF nucleotide-binding site | Q99418 | 46516.66 | 1.09E−03 | 0567; 0549; 0574 |
| | opener) (ARNO protein) (ARF exchange f | | | | |
| 244 | Q9BRD0 (Q9BRD0) Hypothetical protein MGC13125 | Q9BRD0 | 70478.38 | 7.79E−04 | 0574; 0552; 0578 |
| 245 | Q9HCE0 (Q9HCE0) KIAA1632 protein (Fragment) | Q9HCE0 | 165103.1 | 2.18E−03 | 0567; 0541; 0556 |
| 246 | UBN1_HUMAN (Q9NPG3) Ubinuclein (Ubiquitously expressed | Q9NPG3 | 121466.2 | 7.43E−03 | 0541; 0552; 0560 |
| | nuclear protein) (VT4) | | | | |
| 247 | E41LB_HUMAN (Q9Y2J2) Band 4.1-like protein 3 (4.1B) (Differentially | Q9Y2J2 | 120603.1 | 3.21E−03 | 0541; 0571; 0574 |
| | expressed in adenocarcinoma of | | | | |
| 248 | KV3B_HUMAN (P01620) Ig kappa chain V-III region SIE | P01620 | 11767.85 | 4.57E−10 | 0556; 0571 |
| 249 | CO9_HUMAN (P02748) Complement component C9 precursor | P02748 | 63132.78 | 9.29E−06 | 0541; 0535 |
| | [Contains: Complement component C9a; Complemen | | | | |
| 250 | SAP_HUMAN (P07602) Proactivator polypeptide precursor [Contains: | P07602 | 58073.93 | 2.11E−04 | 0574; 0578 |
| | Saposin A (Protein A); Saposin B-V | | | | |
| 251 | CBG_HUMAN (P08185) Corticosteroid-binding globulin precursor | P08185 | 45111.96 | 7.29E−10 | 0574; 0560 |
| | (CBG) (Transcortin) | | | | |
| 252 | CD14_HUMAN (P08571) Monocyte differentiation antigen CD14 | P08571 | 40050.79 | 2.39E−06 | 0541; 0571 |
| | precursor (Myeloid cell-specific leucine- | | | | |
| 253 | AATC_HUMAN (P17174) Aspartate aminotransferase, cytoplasmic | P17174 | 46087.54 | 4.52E−06 | 0560; 0567 |
| | (EC 2.6.1.1) (Transaminase A) (Glutamat | | | | |
| 254 | PGAM1_HUMAN (P18669) Phosphoglycerate mutase 1 (EC 5.4.2.1) | P18669 | 28654.81 | 2.23E−05 | 0560; 0552 |
| | (EC 5.4.2.4) (EC 3.1.3.13) (Phosphoglyc | | | | |
| 255 | NEBU_HUMAN (P20929) Nebulin | P20929 | 772742.8 | 1.73E−03 | 0552; 0571 |
| 256 | ROA2_HUMAN (P22626) Heterogeneous nuclear ribonucleoproteins | P22626 | 37406.69 | 5.25E−07 | 0578; 0560 |
| | A2/B1 (hnRNP A2/hnRNP B1) | | | | |
| 257 | 1433Z_HUMAN (P63104) 14-3-3 protein zeta/delta (Protein kinase C | P63104 | 27727.73 | 1.85E−09 | 0560; 0571 |
| | inhibitor protein 1) (KCIP-1) | | | | |
| 258 | H33_HUMAN (P84243) Histone H3.3 | P84243 | 15187.46 | 3.60E−08 | 0560; 0574 |
| 259 | Q495S9 (Q495S9) Zinc finger protein 560 | Q495S9 | 91061.62 | 4.01E−03 | 0556; 0535 |
| 260 | Q5DP47 (Q5DP47) Tandem-pore-domain potassium channel TREK-1 | Q5DP47 | 24666.08 | 1.46E−03 | 0560; 0578 |
| | splice variant e | | | | |
| 261 | Q6IA31 (Q6IA31) FLJ14154 protein | Q6IA31 | 27415.97 | 4.63E−05 | 0549; 0571 |
| 262 | K0859_HUMAN (Q8N6R0) Protein KIAA0859 | Q8N6R0 | 78717.7 | 1.45E−04 | 0571; 0578 |
| 263 | DDHD1_HUMAN (Q8NEL9) Probable phospholipase DDHD1 (EC | Q8NEL9 | 100372.4 | 3.71E−04 | 0560; 0552 |
| | 3.1.1.—) (DDHD domain protein 1) (Phosphatidi | | | | |
| 264 | Q8TCU4 (Q8TCU4) ALMS1 protein | Q8TCU4 | 460906.3 | 1.73E−03 | 0560; 0571 |
| 265 | NRCAM_HUMAN (Q92823) Neuronal cell adhesion molecule | Q92823 | 143803.8 | 2.93E−07 | 0541; 0574 |
| | precursor (Nr-CAM) (NgCAM-related cell adhesio | | | | |
| 266 | PROL1_HUMAN (Q99935) Proline-rich protein 1 precursor (PRL1) | Q99935 | 22856.06 | 6.78E−10 | 0552 |
| | (Basic proline-rich lacrimal protein) | | | | |
| 267 | APOB_HUMAN (P04114) Apolipoprotein B-100 precursor (Apo B-100) | P04114 | 515241.6 | 1.39E−09 | 0552 |
| | [Contains: Apolipoprotein B-48 (Apo | | | | |
| 268 | ENO1B_HUMAN (Q05524) Alpha-enolase, lung specific (EC 4.2.1.11) | Q05524 | 49446.36 | 2.35E−04 | 0560 |
| | (2-phospho-D-glycerate hydro-lyase) | | | | |
| 269 | K1C13_HUMAN (P13646) Keratin, type I cytoskeletal 13 (Cytokeratin- | P13646 | 49555.45 | 8.40E−11 | 0560 |
| | 13) (CK-13) (Keratin-13) (K13) | | | | |
| 270 | ITIH4_HUMAN (Q14624) Inter-alpha-trypsin inhibitor heavy chain H4 | Q14624 | 103294.1 | 5.77E−09 | 0556; 0552 |
| | precursor (ITI heavy chain H4) (I | | | | |
| 271 | K0317_HUMAN (O15033) Protein KIAA0317 | O15033 | 94162.6 | 7.07E−03 | 0552; 0574 |
| 272 | O43466 (O43466) Hypothetical protein (Fragment) | O43466 | 31230.6 | 7.64E−03 | 0578; 0574 |
| 273 | SF3B1_HUMAN (O75533) Splicing factor 3B subunit 1 (Spliceosome | O75533 | 145723 | 1.09E−03 | 0541; 0560 |
| | associated protein 155) (SAP 155) (S | | | | |
| 274 | RENR_HUMAN (O75787) Renin receptor precursor (Renin/prorenin | O75787 | 38983.34 | 6.70E−07 | 0574; 0541 |
| | receptor) (ATPase, H+ transporting, ly | | | | |
| 275 | LECT1_HUMAN (O75829) Chondromodulin-I precursor (ChM-I) | O75829 | 37078.55 | 6.09E−06 | 0552; 0541 |
| | (Leukocyte cell-derived chemotaxin 1) [Cont | | | | |
| 276 | BPAEA_HUMAN (O94833) Bullous pemphigoid antigen 1, isoforms | O94833 | 590630.3 | 2.08E−03 | 0556; 0574 |
| | 6/9/10 (Trabeculin-beta) (Bullous pemph | | | | |
| 277 | O94873 (O94873) KIAA0775 protein (Fragment) | O94873 | 70117.24 | 7.10E−03 | 0541; 0574 |
| 278 | PRPU_HUMAN (O94906) U5 snRNP-associated 102 kDa protein (U5- | O94906 | 106857.9 | 1.54E−03 | 0571; 0567 |
| | 102 kDa protein) | | | | |
| 279 | ZN202_HUMAN (O95125) Zinc finger protein 202 | O95125 | 74673.56 | 6.83E−03 | 0560; 0578 |
| 280 | RECK_HUMAN (O95980) Reversion-inducing cysteine-rich protein | O95980 | 106385.6 | 2.21E−03 | 0571; 0578 |
| | with Kazal motifs precursor (hRECK) (S | | | | |
| 281 | LDHA-HUMAN (P00338) L-lactate dehydrogenase A chain (EC | P00338 | 36534.34 | 1.35E−04 | 0560; 0556 |
| | 1.1.1.27) (LDH-A) (LDH muscle subunit) (LDH | | | | |
| 282 | ASSY_HUMAN (P00966) Argininosuccinate synthase (EC 6.3.4.5) | P00966 | 46501.01 | 5.87E−03 | 0571; 0541 |
| | (Citrulline--aspartate ligase) | | | | |
| 283 | CYTS_HUMAN (P01036) Cystatin S precursor (Salivary acidic protein | P01036 | 16203.97 | 3.31E−06 | 0552; 0571 |
| | 1) (Cystatin SA-III) | | | | |
| 284 | KV2A_HUMAN (P01614) Ig kappa chain V-II region Cum | P01614 | 12668.32 | 9.27E−06 | 0578; 0552 |
| 285 | HV3E_HUMAN (P01766) Ig heavy chain V-III region BRO | P01766 | 13218.4 | 2.37E−11 | 0556; 0571 |
| 286 | SCG1_HUMAN (P05060) Secretogranin-1 precursor (Secretogranin I) | P05060 | 78199.14 | 7.52E−06 | 0571; 0560 |
| | (SgI) (Chromogranin B) (CgB) [Conta | | | | |
| 287 | TBB2_HUMAN (P07437) Tubulin beta-2 chain | P07437 | 49639.01 | 1.29E−10 | 0578; 0560 |
| 288 | CATB_HUMAN (P07858) Cathepsin B precursor (EC 3.4.22.1) | P07858 | 37796.84 | 2.93E−05 | 0578; 0567 |
| | (Cathepsin B1) (APP secretase) (APPS) [Cont | | | | |
| 289 | THIK_HUMAN (P09110) 3-ketoacyl-CoA thiolase, peroxisomal | P09110 | 44263.91 | 8.00E−04 | 0571; 0560 |
| | precursor (EC 2.3.1.16) (Beta-ketothiolase | | | | |
| 290 | K2C4_HUMAN (P19013) Keratin, type II cytoskeletal 4 (Cytokeratin-4) | P19013 | 57259.96 | 1.64E−05 | 0578; 0560 |
| | (CK-4) (Keratin-4) (K4) | | | | |
| 291 | MT3_HUMAN (P25713) Metallothionein-3 (MT-3) (Metallothionein-III) | P25713 | 6921.488 | 7.21E−06 | 0560; 0552 |
| | (MT-III) (Growth inhibitory facto | | | | |
| 292 | TKT_HUMAN (P29401) Transketolase (EC 2.2.1.1) (TK) | P29401 | 67834.88 | 9.46E−05 | 0560; 0578 |
| 293 | I5P2_HUMAN (P32019) Type II inositol-1,4,5-trisphosphate 5- | P32019 | 112750.1 | 4.50E−03 | 0560; 0578 |
| | phosphatase precursor (EC 3.1.3.36) (Pho | | | | |
| 294 | ICAM3_HUMAN (P32942) Intercellular adhesion molecule 3 precursor | P32942 | 59345.85 | 3.49E−03 | 0571; 0560 |
| | (ICAM-3) (ICAM-R) (CDw50) (CD50 an | | | | |
| 295 | K22E_HUMAN (P35908) Keratin, type II cytoskeletal 2 epidermal | P35908 | 65825.37 | 3.38E−06 | 0574; 0560 |
| | (Cytokeratin-2e) (K2e) (CK 2e) | | | | |
| 296 | ALU6_HUMAN (P39193) Alu subfamily SP sequence contamination | P39193 | 64656.59 | 9.60E−04 | 0567; 0549 |
| | warning entry | | | | |
| 297 | BRCA2_HUMAN (P51587) Breast cancer type 2 susceptibility protein | P51587 | 383984.9 | 2.59E−03 | 0556; 0571 |
| | (Fanconi anemia group D1 protein) | | | | |
| 298 | ELL_HUMAN (P55199) RNA polymerase II elongation factor ELL | P55199 | 68222.91 | 1.98E−03 | 0578; 0549 |
| | (Eleven-nineteen lysine-rich leukemia pr | | | | |
| 299 | ACTB_HUMAN (P60709) Actin, cytoplasmic 1 (Beta-actin) | P60709 | 41709.74 | 6.35E−04 | 0541; 0574 |
| 300 | PGBM_HUMAN (P98160) Basement membrane-specific heparan | P98160 | 468528.2 | 4.97E−08 | 0541; 0574 |
| | sulfate proteoglycan core protein precursor | | | | |
| 301 | TAP1_HUMAN (Q03518) Antigen peptide transporter 1 (APT1) | Q03518 | 80913.73 | 2.20E−03 | 0571; 0578 |
| | (Peptide transporter TAP1) (ATP-binding ca | | | | |
| 302 | U33K_HUMAN (Q04323) UBA/UBX 33.3 kDa protein | Q04323 | 33304.96 | 6.51E−03 | 0560; 0556 |
| 303 | ZN11B_HUMAN (Q06732) Zinc finger protein 11B | Q06732 | 90624.22 | 4.57E−03 | 0535; 0574 |
| 304 | SOX4_HUMAN (Q06945) Transcription factor SOX-4 | Q06945 | 47233.82 | 3.00E−03 | 0574; 0571 |
| 305 | TRDN_HUMAN (Q13061) Triadin | Q13061 | 81374.51 | 1.48E−03 | 0574; 0578 |
| 306 | I10R1_HUMAN (Q13651) Interleukin-10 receptor alpha chain | Q13651 | 62864.42 | 1.84E−04 | 0535; 0556 |
| | precursor (IL-10R-A) (IL-10R1) | | | | |
| 307 | Q14467 (Q14467) KIAA0068 protein (Fragment) | Q14467 | 147040.6 | 6.74E−03 | 0535; 0578 |
| 308 | KRHB1_HUMAN (Q14533) Keratin, type II cuticular Hb1 (Hair keratin, | Q14533 | 54935.82 | 7.26E−04 | 0552; 0535 |
| | type II Hb1) (ghHKb1) (ghHb1) (M | | | | |
| 309 | GOGB1_HUMAN (Q14789) Golgi autoantigen, golgin subfamily B | Q14789 | 375846.5 | 5.99E−03 | 0541; 0549 |
| | member 1 (Giantin) (Macrogolgin) (Golgi | | | | |
| 310 | ZMY11_HUMAN (Q15326) Zinc finger MYND domain containing | Q15326 | 66160.48 | 2.74E−03 | 0552; 0571 |
| | protein 11 (Adenovirus 5 E1A-binding protei | | | | |
| 311 | Q15786 (Q15786) Testis calpastatin | Q15786 | 47056.18 | 3.44E−03 | 0571; 0574 |
| 312 | ITSN1_HUMAN (Q15811) Intersectin-1 (SH3 domain-containing | Q15811 | 195410 | 1.11E−03 | 0556; 0574 |
| | protein 1A) (SH3P17) | | | | |
| 313 | DUS5_HUMAN (Q16690) Dual specificity protein phosphatase 5 (EC | Q16690 | 42080.39 | 4.50E−04 | 0556; 0552 |
| | 3.1.3.48) (EC 3.1.3.16) (Dual specif | | | | |
| 314 | Q53LP3 (Q53LP3) Hypothetical protein FLJ21870 (C2orf26 protein) | Q53LP3 | 55638.11 | 1.89E−03 | 0552; 0567 |
| 315 | Q53RD9 (Q53RD9) Hypothetical protein FLJ37440 | Q53RD9 | 47344.78 | 3.38E−03 | 0560; 0552 |
| 316 | Q53TQ3 (Q53TQ3) Hypothetical protein FLJ20309 | Q53TQ3 | 100659.6 | 6.83E−03 | 0571; 0552 |
| 317 | Q5T0F8 (Q5T0F8) Novel protein | Q5T0F8 | 93537.23 | 5.83E−03 | 0571; 0574 |
| 318 | Q5T1V5 (Q5T1V5) Novel protein (Fragment) | Q5T1V5 | 17690.71 | 8.01E−03 | 0578; 0567 |
| 319 | Q5VYM8 (Q5VYM8) Unc-13 homolog B (C. elegans) | Q5VYM8 | 180563.2 | 4.01E−03 | 0574; 0578 |
| 320 | Q68DL7 (Q68DL7) Hypothetical protein DKFZp781G0119 | Q68DL7 | 77181.3 | 1.62E−03 | 0567; 0541 |
| 321 | Q6EKI8 (Q6EKI8) General transcription factor II i repeat domain 2 | Q6EKI8 | 107162.2 | 4.27E−03 | 0535; 0556 |
| | alpha | | | | |
| 322 | Q6EMK4 (Q6EMK4) Vasorin | Q6EMK4 | 71667.73 | 1.19E−09 | 0571; 0541 |
| 323 | Q6P2S8 (Q6P2S8) Hypothetical protein | Q6P2S8 | 15673.02 | 2.81E−03 | 0552; 0556 |
| 324 | Q6RJU1 (Q6RJU1) RhoGTPase regulating protein variant | Q6RJU1 | 130153.4 | 4.43E−03 | 0541; 0552 |
| | ARHGAP20-1be | | | | |
| 325 | Q6ZMX4 (Q6ZMX4) Hypothetical protein FLJ16611 | Q6ZMX4 | 83550.26 | 5.47E−03 | 0574; 0541 |
| 326 | Q6ZRP4 (Q6ZRP4) Hypothetical protein FLJ46205 | Q6ZRP4 | 135088.2 | 3.69E−03 | 0571; 0556 |
| 327 | Q6ZVQ3 (Q6ZVQ3) Hypothetical protein FLJ42220 | Q6ZVQ3 | 17595.21 | 1.21E−04 | 0535; 0578 |
| 328 | Q6ZW11 (Q6ZW11) Hypothetical protein FLJ41805 | Q6ZW11 | 13506.89 | 2.84E−03 | 0574; 0552 |
| 329 | UN13D_HUMAN (Q70J99) Unc-13 homolog D (Munc13-4) | Q70J99 | 123204.8 | 9.94E−04 | 0571; 0567 |
| 330 | Q7Z402 (Q72402) Transmembrane channel-like protein 7 | Q7Z402 | 83447.49 | 5.08E−04 | 0574; 0552 |
| 331 | ABCAC_HUMAN (Q86UK0) ATP-binding cassette sub-family A | Q86UK0 | 293060.7 | 1.27E−04 | 0567; 0556 |
| | member 12 (ATP-binding cassette transporter | | | | |
| 332 | Q86VJ0 (Q86VJ0) Aaa-protein | Q86VJ0 | 77428.84 | 3.96E−03 | 0556; 0549 |
| 333 | Q86VW2 (Q86VW2) RAC/CDC42 exchange factor, isoform 1 | Q86VW2 | 63989.59 | 2.94E−03 | 0560; 0578 |
| | (RAC/CDC42/Rho exchange factor GEFT) | | | | |
| 334 | Q86W42 (Q86W42) WD repeat domain 58 | Q86W42 | 37511.05 | 4.61E−03 | 0567; 0571 |
| 335 | Q8N2X6 (Q8N2X6) LOC116349 protein | Q8N2X6 | 12653.28 | 1.20E−03 | 0541; 0578 |
| 336 | Q8N6W7 (Q8N6W7) PML-RARA regulated adaptor molecule 1 | Q8N6W7 | 73950.18 | 3.73E−03 | 0571; 0578 |
| 337 | Q8NBB8 (Q8NBB8) Hypothetical protein FLJ33743 | Q8NBB8 | 53627.89 | 6.15E−04 | 0541; 0571 |
| 338 | GALT6_HUMAN (Q8NCL4) Polypeptide N- | Q8NCL4 | 71113.15 | 4.27E−03 | 0535; 0571 |
| | acetylgalactosaminyltransferase 6 (EC 2.4.1.41) (Protein-UDP ace | | | | |
| 339 | LIPB2_HUMAN (Q8ND30) Liprin-beta 2 (Protein tyrosine phosphatase | Q8ND30 | 98383.37 | 8.29E−04 | 0578; 0567 |
| | receptor type f polypeptide-intera | | | | |
| 340 | DYH5_HUMAN (Q8TE73) Ciliary dynein heavy chain 5 (Axonemal | Q8TE73 | 528667.8 | 4.96E−03 | 0560; 0556 |
| | beta dynein heavy chain 5) (HL1) | | | | |
| 341 | NEUR4_HUMAN (Q8WWR8) Sialidase 4 (EC 3.2.1.18) (N-acetyl- | Q8WWR8 | 51639.02 | 3.08E−04 | 0571; 0560 |
| | alpha-neuraminidase 4) | | | | |
| 342 | RYR2_HUMAN (Q92736) Ryanodine receptor 2 (Cardiac muscle-type | Q92736 | 564136.1 | 2.95E−03 | 0574; 0541 |
| | ryanodine receptor) (RyR2) (RYR-2) (C | | | | |
| 343 | COR2A_HUMAN (Q92828) Coronin-2A (WD repeat-containing protein | Q92828 | 59725.43 | 7.02E−03 | 0574; 0556 |
| | 2) (IR10) | | | | |
| 344 | SNPC3_HUMAN (Q92966) snRNA-activating protein complex subunit | Q92966 | 46722.65 | 8.82E−03 | 0578; 0571 |
| | 3 (SNAPc subunit 3) (snRNA-activating | | | | |
| 345 | Q969T7 (Q969T7) Hypothetical protein MGC20781 | Q969T7 | 33551.02 | 5.75E−03 | 0574; 0556 |
| 346 | Q96E25 (Q96E25) ZNF503 protein | Q96E25 | 59255.39 | 9.31E−04 | 0578; 0541 |
| 347 | Q96EK0 (Q96EK0) Hypothetical protein MGC4655 | Q96EK0 | 41865.86 | 5.12E−03 | 0552; 0541 |
| 348 | Q96IV6 (Q96IV6) C5orf4 protein | Q96IV6 | 38976.07 | 1.54E−04 | 0541; 0571 |
| 349 | Q96JD0 (Q96JD0) Amyloid lambda 6 light chain variable region SAR | Q96JD0 | 12286.89 | 3.67E−05 | 0556; 0535 |
| | (Fragment) | | | | |
| 350 | K1849_HUMAN (Q96JH8) Protein KIAA1849 | Q96JH8 | 116630 | 3.22E−03 | 0574; 0560 |
| 351 | ACON_HUMAN (Q99798) Aconitate hydratase, mitochondrial | Q99798 | 85372.02 | 1.20E−03 | 0541; 0578 |
| | precursor (EC 4.2.1.3) (Citrate hydro-lyase) | | | | |
| 352 | AKAP9_HUMAN (Q99996) A-kinase anchor protein 9 (Protein kinase | Q99996 | 453386.1 | 4.17E−03 | 0549; 0571 |
| | A anchoring protein 9) (PRKA9) (A-ki | | | | |
| 353 | SIN1_HUMAN (Q9BPZ7) Stress-activated map kinase interacting | Q9BPZ7 | 59086.05 | 6.16E−03 | 0567; 0541 |
| | protein 1 (SAPK interacting protein 1) | | | | |
| 354 | Q9BR77 (Q9BR77) Hypothetical protein MGC13183 (Hypothetical | Q9BR77 | 57450.07 | 2.90E−04 | 0541; 0556 |
| | protein FLJ14732) | | | | |
| 355 | DATF1_HUMAN (Q9BTC0) Death-associated transcription factor 1 | Q9BTC0 | 129070.5 | 2.77E−03 | 0552; 0567 |
| 356 | Q9BZV3 (Q9BZV3) Interphotoreceptor matrix proteoglycan 200 | Q9BZV3 | 138545 | 6.65E−05 | 0541; 0574 |
| 357 | DYI4_HUMAN (Q9GZS0) Dynein intermediate chain 2, axonemal | Q9GZS0 | 68807.61 | 1.67E−03 | 0571; 0549 |
| | (Axonemal dynein intermediate chain 2) | | | | |
| 358 | Q9H0C8 (Q9H0C8) Hypothetical protein DKFZp434J2031 (Integrin- | Q9H0C8 | 42879.83 | 8.69E−03 | 0552; 0567 |
| | linked kinase-associated serine/threon | | | | |
| 359 | ZN335_HUMAN (Q9H4Z2) Zinc finger protein 335 | Q9H4Z2 | 144802 | 1.44E−03 | 0541; 0567 |
| 360 | RANB3_HUMAN (Q9H6Z4) Ran-binding protein 3 (RanBP3) | Q9H6Z4 | 60173.32 | 1.48E−03 | 0552; 0556 |
| 361 | LPHN3_HUMAN (Q9HAR2) Latrophilin-3 precursor (Calcium- | Q9HAR2 | 161709.3 | 5.64E−03 | 0549; 0541 |
| | independent alpha-latrotoxin receptor 3) (Lec | | | | |
| 362 | RENT2_HUMAN (Q9HAU5) Regulator of nonsense transcripts 2 | Q9HAU5 | 147716.9 | 9.67E−04 | 0560; 0567 |
| | (Nonsense mRNA reducing factor 2) (Up-fram | | | | |
| 363 | Q9HB10 (Q9HB10) Pinch-2 protein (Hypothetical protein LOC96626) | Q9HB10 | 13242.23 | 3.83E−03 | 0567; 0552 |
| | (LIM and senescent cell antigen-lik | | | | |
| 364 | RDH14_HUMAN (Q9HBH5) Retinol dehydrogenase 14 (EC 1.1.1.—) | Q9HBH5 | 36841.38 | 5.57E−03 | 0578; 0574 |
| | (Alcohol dehydrogenase PAN2) | | | | |
| 365 | Q9HC06 (Q9HC06) Cd002 protein | Q9HC06 | 43466.54 | 4.76E−03 | 0560; 0556 |
| 366 | ITM2C_HUMAN (Q9NQX7) Integral membrane protein 2C | Q9NQX7 | 30204.46 | 1.96E−03 | 0578; 0556 |
| | (Transmembrane protein BRI3) (Cerebral protein 14 | | | | |
| 367 | Q9NS87 (Q9NS87) Kinesin-like protein 2 | Q9NS87 | 160060.4 | 6.49E−03 | 0552; 0549 |
| 368 | Q9NXU1 (Q9NXU1) Hypothetical protein FLJ20055 | Q9NXU1 | 46686.5 | 8.52E−03 | 0567; 0552 |
| 369 | UGGG1_HUMAN (Q9NYU2) UDP-glucose:glycoprotein | Q9NYU2 | 174866.5 | 1.64E−03 | 0552; 0556 |
| | glucosyltransferase 1 precursor (EC 2.4.1.—) (UDP-glu | | | | |
| 370 | VPS18_HUMAN (Q9P253) Vacuolar protein sorting 18 (hVPS18) | Q9P253 | 110115.9 | 1.55E−03 | 0574; 0552 |
| 371 | TRABD_HUMAN (Q9UGI0) TRABID protein (Zinc finger Ran-binding | Q9UGI0 | 80855.84 | 7.58E−03 | 0541; 0556 |
| | domain containing 1) | | | | |
| 372 | DCTN4_HUMAN (Q9UJW0) Dynactin subunit 4 (Dynactin subunit | Q9UJW0 | 52303.69 | 2.67E−03 | 0535; 0556 |
| | p62) | | | | |
| 373 | Q9ULH9 (Q9ULH9) KIAA1241 protein (Fragment) | Q9ULH9 | 97158.45 | 3.40E−04 | 0567; 0574 |
| 374 | SATB2_HUMAN (Q9UPW6) DNA-binding protein SATB2 (Special AT- | Q9UPW6 | 82504.16 | 8.57E−04 | 0556; 0535 |
| | rich sequence-binding protein 2) | | | | |
| 375 | LZTS1_HUMAN (Q9Y250) Leucine zipper putative tumor suppressor | Q9Y250 | 66440.97 | 3.05E−04 | 0552; 0549 |
| | 1 (F37/esophageal cancer-related gene | | | | |
| 376 | WBP11_HUMAN (Q9Y2W2) WW domain-binding protein 11 (WBP- | Q9Y2W2 | 69954.12 | 2.54E−04 | 0549; 0541 |
| | 11) (SH3 domain-binding protein SNP70) (Npw3 | | | | |
| 377 | TF3C3_HUMAN (Q9Y5Q9) General transcription factor 3C | Q9Y5Q9 | 101207.8 | 4.66E−03 | 0571; 0574 |
| | polypeptide 3 (Transcription factor IIIC-gamma | | | | |
| 378 | Q5MJ67 (Q5MJ67) CMYA3 | Q5MJ67 | 378997.3 | 1.45E−04 | 0552 |
| 379 | Q6N095 (Q6N095) Hypothetical protein DKFZp686K03196 | Q6N095 | 52327.14 | 8.86E−04 | 0556 |
| 380 | O15422 (O15422) Probable zinc finger protein H101 (Fragment) | O15422 | 13289.94 | 4.52E−05 | 0552 |
| 381 | HNRPR_HUMAN (O43390) Heterogeneous nuclear ribonucleoprotein | O43390 | 70899.22 | 5.51E−04 | 0567 |
| | R (hnRNP R) | | | | |
| 382 | O43532 (O43532) RIG-like 7-1 | O43532 | 19272.97 | 4.33E−03 | 0571 |
| 383 | PTCD1_HUMAN (O75127) Pentatricopeptide repeat protein 1 | O75127 | 78805.96 | 8.20E−04 | 0574 |
| 384 | AL1A1_HUMAN (P00352) Retinal dehydrogenase 1 (EC 1.2.1.36) | P00352 | 54695.95 | 2.56E−07 | 0549 |
| | (RalDH1) (RALDH 1) (Aldehyde dehydrogena | | | | |
| 385 | CYTB_HUMAN (P04080) Cystatin B (Liver thiol proteinase inhibitor) | P04080 | 11132.59 | 1.05E−09 | 0560 |
| | (CPI-B) (Stefin B) | | | | |
| 386 | SODM_HUMAN (P04179) Superoxide dismutase [Mn], mitochondrial | P04179 | 24706.56 | 3.02E−06 | 0560 |
| | precursor (EC 1.15.1.1) | | | | |
| 387 | KV3I_HUMAN (P04433) Ig kappa chain V-III region VG precursor | P04433 | 12567.31 | 1.61E−05 | 0556 |
| | (Fragment) | | | | |
| 388 | HV2I_HUMAN (P06331) Ig heavy chain V-II region ARH-77 precursor | P06331 | 16218.12 | 7.95E−04 | 0560 |
| 389 | CRGB_HUMAN (P07316) Gamma crystallin B (Gamma crystallin 1-2) | P07316 | 20762.91 | 8.74E−06 | 0549 |
| 390 | GSTM1_HUMAN (P09488) Glutathione S-transferase Mu 1 (EC | P09488 | 25564.02 | 2.53E−04 | 0560 |
| | 2.5.1.18) (GSTM1-1) (GST class-mu 1) (GSTM1 | | | | |
| 391 | ROA1_HUMAN (P09651) Heterogeneous nuclear ribonucleoprotein | P09651 | 38691.05 | 4.89E−07 | 0560 |
| | A1 (Helix-destabilizing protein) (Singl | | | | |
| 392 | CMGA_HUMAN (P10645) Chromogranin A precursor (CgA) (Pituitary | P10645 | 50699.73 | 1.46E−04 | 0541 |
| | secretory protein I) (SP-I) [Contains | | | | |
| 393 | ENOB_HUMAN (P13929) Beta-enolase (EC 4.2.1.11) (2-phospho-D- | P13929 | 46826.33 | 7.77E−09 | 0578 |
| | glycerate hydro-lyase) (Muscle-specific | | | | |
| 394 | PTN2_HUMAN (P17706) Tyrosine-protein phosphatase, non-receptor | P17706 | 48497.45 | 4.46E−04 | 0552 |
| | type 2 (EC 3.1.3.48) (T-cell protein | | | | |
| 395 | PTMS_HUMAN (P20962) Parathymosin | P20962 | 11392.15 | 1.96E−06 | 0578 |
| 396 | COIA1_HUMAN (P39060) Collagen alpha 1(XVIII) chain precursor | P39060 | 153731.9 | 7.65E−04 | 0552 |
| | [Contains: Endostatin] | | | | |
| 397 | TSC2_HUMAN (P49815) Tuberin (Tuberous sclerosis 2 protein) | P49815 | 200621.4 | 1.76E−05 | 0571 |
| 398 | MECP2_HUMAN (P51608) Methyl-CpG-binding protein 2 (MeCP-2 | P51608 | 52408.71 | 2.49E−03 | 0552 |
| | protein) (MeCP2) | | | | |
| 399 | PUR1_HUMAN (Q06203) Amidophosphoribosyltransferase precursor | Q06203 | 57362.45 | 1.67E−03 | 0578 |
| | (EC 2.4.2.14) (Glutamine phosphoribosy | | | | |
| 400 | LTBP2_HUMAN (Q14767) Latent transforming growth factor-beta- | Q14767 | 194935.4 | 1.09E−04 | 0578 |
| | binding protein 2 precursor (LTBP-2) | | | | |
| 401 | Q4UJ75 (Q4UJ75) Novel protein similar to ankyrin repeat domain 20A | Q4UJ75 | 94090.2 | 1.31E−03 | 0578 |
| | (ANKRD20A) | | | | |
| 402 | Q5GC90 (Q5GC90) HERC6 splice variant | Q5GC90 | 111012.2 | 7.32E−03 | 0549 |
| 403 | Q5H9Q6 (Q5H9Q6) Hypothetical protein DKFZp781N0678 | Q5H9Q6 | 64496.32 | 2.49E−03 | 0556 |
| 404 | Q5SGD5 (Q5SGD5) Maguin-like protein variant II (Maguin-like protein | Q5SGD5 | 61848.88 | 4.57E−03 | 0552 |
| | variant III) (Maguin-like prote | | | | |
| 405 | ZCHC6_HUMAN (Q5VYS8) Zinc finger CCHC domain containing | Q5VYS8 | 171121 | 3.70E−05 | 0549 |
| | protein 6 | | | | |
| 406 | Q6GMX7 (Q6GMX7) Hypothetical protein | Q6GMX7 | 51598.66 | 7.72E−04 | 0560 |
| 407 | Q6NUI2 (Q6NUI2) LOC150763 protein | Q6NUI2 | 87507.18 | 9.25E−04 | 0571 |
| 408 | Q6PJ73 (Q6PJ73) DPF1 protein (Fragment) | Q6PJ73 | 47450.2 | 1.65E−03 | 0578 |
| 409 | ATAD2_HUMAN (Q6PL18) ATPase family AAA domain containing | Q6PL18 | 158456 | 6.93E−03 | 0574 |
| | protein 2 | | | | |
| 410 | Q6ZW49 (Q6ZW49) Hypothetical protein FLJ41606 | Q6ZW49 | 117615.6 | 5.23E−03 | 0552 |
| 411 | Q8IY85 (Q8IY85) Hypothetical protein FLJ40342 | Q8IY85 | 110027.5 | 2.60E−04 | 0560 |
| 412 | CI126_HUMAN (Q8N9R8) Protein C9orf126 | Q8N9R8 | 70385.07 | 4.09E−03 | 0578 |
| 413 | Q8NAI5 (Q8NAI5) Hypothetical protein FLJ35318 | Q8NAI5 | 25654.67 | 9.38E−04 | 0552 |
| 414 | ACBD4_HUMAN (Q8NC06) Acyl-CoA binding domain containing | Q8NC06 | 30287.72 | 3.34E−03 | 0541 |
| | protein 4 | | | | |
| 415 | AT8B4_HUMAN (Q8TF62) Probable phospholipid-transporting | Q8TF62 | 135853.6 | 1.41E−03 | 0549 |
| | ATPase IM (EC 3.6.3.1) (ATPase class I type | | | | |
| 416 | FAM3C_HUMAN (Q92520) Protein FAM3C precursor (Protein | Q92520 | 24664.59 | 3.85E−03 | 0574 |
| | GS3786) | | | | |
| 417 | K1H5_HUMAN (Q92764) Keratin, type I cuticular Ha5 (Hair keratin, | Q92764 | 47566.21 | 1.49E−04 | 0549 |
| | type I Ha5) | | | | |
| 418 | SFRP3_HUMAN (Q92765) Secreted frizzled-related protein 3 | Q92765 | 36230.29 | 1.65E−04 | 0560 |
| | precursor (sFRP-3) (Frizzled-related prote | | | | |
| 419 | RL10L_HUMAN (Q96L21) 60S ribosomal protein L10-like | Q96L21 | 24371.68 | 3.34E−03 | 0535 |
| 420 | Q9BRQ8 (Q9BRQ8) Apoptosis-inducing factor (AIF)-like | Q9BRQ8 | 40501.28 | 2.40E−03 | 0552 |
| | mitchondrion-associated inducer of death (P53- | | | | |
| 421 | SPTN4_HUMAN (Q9H254) Spectrin beta chain, brain 3 (Spectrin, | Q9H254 | 288806.1 | 1.24E−03 | 0552 |
| | non-erythroid beta chain 3) (Beta-IV s | | | | |
| 422 | Q9H7Z0 (Q9H7Z0) Hypothetical protein FLJ14058 | Q9H7Z0 | 24063.12 | 1.23E−03 | 0574 |
| 423 | AT131_HUMAN (Q9HD20) Probable cation-transporting ATPase | Q9HD20 | 132869.9 | 2.33E−03 | 0552 |
| | 13A1 (EC 3.6.3.—) | | | | |
| 424 | SEM4B_HUMAN (Q9NPR2) Semaphorin-4B precursor | Q9NPR2 | 92134.84 | 1.03E−06 | 0574 |
| 425 | Q9Y348 (Q9Y348) Hypothetical protein | Q9Y348 | 12406.69 | 7.64E−03 | 0549 |
| 426 | Q6FGL5 (Q6FGL5) LCN2 protein (Fragment) | Q6FGL5 | 22547.66 | 2.11E−10 | 0552 |
| 427 | MUCB_HUMAN (P04220) Ig mu heavy chain disease protein (BOT) | P04220 | 43030.31 | 5.96E−07 | 0578 |
| 428 | K2C1_HUMAN (P04264) Keratin, type II cytoskeletal 1 (Cytokeratin-1) | P04264 | 65846.88 | 8.29E−05 | 0574 |
| | (CK-1) (Keratin-1) (K1) (67 kDa | | | | |
| 429 | RABP1_HUMAN (P29762) Cellular retinoic acid binding protein 1 | P29762 | 15424.71 | 1.13E−06 | 0560 |
| | (Cellular retinoic acid binding prote | | | | |
| 430 | AL3A1_HUMAN (P30838) Aldehyde dehydrogenase, dimeric NADP- | P30838 | 50346.91 | 7.08E−03 | 0578 |
| | preferring (EC 1.2.1.5) (ALDH class 3) (A | | | | |
| 431 | DPOD2_HUMAN (P49005) DNA polymerase delta subunit 2 (EC | P49005 | 51257.05 | 4.77E−03 | 0556 |
| | 2.7.7.7) (DNA polymerase delta subunit p50) | | | | |
| 432 | QORX_HUMAN (Q53FA7) Putative quinone oxidoreductase (EC 1.—.—.—) | Q53FA7 | 35513.59 | 1.04E−03 | 0552 |
| | (Tumor protein p53-inducible prote | | | | |
| 433 | Q65ZC8 (Q65ZC8) Single-chain Fv (Fragment) | Q65ZC8 | 26110.55 | 1.26E−05 | 0535 |
| 434 | AP3B1_HUMAN (O00203) Adapter-related protein complex 3 beta 1 | O00203 | 121274.4 | 8.21E−03 | 0567 |
| | subunit (Beta3A-adaptin) (Adaptor pro | | | | |
| 435 | PSD12_HUMAN (O00232) 26S proteasome non-ATPase regulatory | O00232 | 52739.59 | 8.95E−03 | 0552 |
| | subunit 12 (26S proteasome regulatory sub | | | | |
| 436 | BMR1B_HUMAN (O00238) Bone morphogenetic protein receptor type | O00238 | 56893.71 | 7.71E−03 | 0552 |
| | IB precursor (EC 2.7.1.37) | | | | |
| 437 | WWP2_HUMAN (O00308) Nedd-4-like E3 ubiquitin-protein ligase | O00308 | 98850.05 | 4.72E−03 | 0556 |
| | WWP2 (EC 6.3.2.—) (WW domain-containing | | | | |
| 438 | O00319 (O00319) WUGSC:H_DJ525N14.1 protein | O00319 | 74438.16 | 2.51E−03 | 0571 |
| 439 | ODPX_HUMAN (O00330) Pyruvate dehydrogenase protein X | O00330 | 54088.89 | 5.59E−03 | 0571 |
| | component, mitochondrial precursor (Dihydrolip | | | | |
| 440 | PLK4_HUMAN (O00444) Serine/threonine-protein kinase PLK4 (EC | O00444 | 108903.3 | 3.85E−03 | 0541 |
| | 2.7.1.37) (Polo-like kinase 4) (PLK-4) | | | | |
| 441 | O00461 (O00461) 130 kD Golgi-localized phosphoprotein | O00461 | 81831.17 | 7.79E−03 | 0567 |
| 442 | AGRN_HUMAN (O00468) Agrin precursor | O00468 | 214747.2 | 1.16E−03 | 0571 |
| 443 | NPT3_HUMAN (O00624) Sodium-dependent phosphate transport | O00624 | 47319.56 | 4.49E−04 | 0567 |
| | protein 3 (Sodium/phosphate cotransporter | | | | |
| 444 | CUTL2_HUMAN (O14529) Homeobox protein Cux-2 (Cut-like 2) | O14529 | 154100.3 | 6.33E−03 | 0556 |
| 445 | O14712 (O14712) Cell cycle progression restoration 8 protein | O14712 | 44292.57 | 9.69E−03 | 0549 |
| 446 | CHK1_HUMAN (O14757) Serine/threonine-protein kinase Chk1 (EC | O14757 | 54384.94 | 6.24E−03 | 0571 |
| | 2.7.1.37) | | | | |
| 447 | TRI66_HUMAN (O15016) Tripartite motif protein 66 | O15016 | 134317.6 | 7.00E−03 | 0560 |
| 448 | DCAK1_HUMAN (O15075) Serine/threonine-protein kinase | O15075 | 82172.98 | 9.71E−03 | 0541 |
| | DCAMKL1 (EC 2.7.1.37) (Doublecortin-like and C | | | | |
| 449 | IKKA_HUMAN (O15111) Inhibitor of nuclear factor kappa-B kinase | O15111 | 84599.03 | 6.24E−03 | 0560 |
| | alpha subunit (EC 2.7.1.—) (I kappa- | | | | |
| 450 | VILL_HUMAN (O15195) Villin-like protein | O15195 | 95847.38 | 3.84E−04 | 0578 |
| 451 | VGF_HUMAN (O15240) Neurosecretory protein VGF precursor | O15240 | 67246.72 | 5.02E−03 | 0571 |
| 452 | O15504 (O15504) Hypothetical protein CG1 | O15504 | 44844.05 | 9.61E−03 | 0556 |
| 453 | MSH5_HUMAN (O43196) MutS protein homolog 5 | O43196 | 92816.59 | 5.63E−04 | 0556 |
| 454 | O43305 (O43305) KIAA0421 protein (Fragment) | O43305 | 223741 | 4.29E−03 | 0567 |
| 455 | O43314 (O43314) KIAA0433 protein (Fragment) | O43314 | 141615.9 | 2.82E−03 | 0556 |
| 456 | O43686 (O43686) Interphotoreceptor matrix proteoglycan 150 | O43686 | 89331.24 | 1.60E−09 | 0541 |
| | (OTTHUMP00000016767) | | | | |
| 457 | O43857 (O43857) Fas-ligand associated factor 2 (Fragment) | O43857 | 16002.3 | 1.43E−03 | 0574 |
| 458 | H6ST1_HUMAN (O60243) Heparan-sulfate 6-O-sulfotransferase 1 | O60243 | 48164.27 | 7.29E−03 | 0535 |
| | (EC 2.8.2.—) (HS6ST-1) | | | | |
| 459 | KPRB_HUMAN (O60256) Phosphoribosyl pyrophosphate synthetase- | O60256 | 40899.39 | 1.83E−04 | 0552 |
| | associated protein 2 (PRPP synthetase-a | | | | |
| 460 | O60276 (O60276) KIAA0523 protein (Fragment) | O60276 | 53723.5 | 7.19E−04 | 0560 |
| 461 | O60350 (O60350) Sodium bicarbonate cotransporter2 | O60350 | 114131.7 | 1.42E−03 | 0567 |
| 462 | O60502 (O60502) Meningioma-expressed antigen 5 | O60502 | 102848.9 | 3.70E−03 | 0549 |
| 463 | VINEX_HUMAN (O60504) Vinexin (Sorbin and SH3 domain- | O60504 | 75283 | 5.71E−03 | 0552 |
| | containing protein 3) (SH3-containing adapter m | | | | |
| 464 | M6PBP_HUMAN (O60664) Mannose-6-phosphate receptor binding | O60664 | 47017.98 | 7.29E−03 | 0552 |
| | protein 1 (Cargo selection protein TIP47) | | | | |
| 465 | JAK2_HUMAN (O60674) Tyrosine-protein kinase JAK2 (EC | O60674 | 130589.7 | 8.75E−03 | 0560 |
| | 2.7.1.112) (Janus kinase 2) (JAK-2) | | | | |
| 466 | O60687 (O60687) Sushi-repeat-containing protein, X-linked 2 | O60687 | 52937.79 | 1.02E−03 | 0556 |
| 467 | FMO6_HUMAN (O60774) Putative dimethylaniline monooxygenase | O60774 | 61251.68 | 1.50E−04 | 0549 |
| | [N-oxide-forming] 6 (EC 1.14.13.8) (Flav | | | | |
| 468 | HNRCL_HUMAN (O60812) Heterogeneous nuclear ribonucleoprotein | O60812 | 32122.71 | 6.44E−05 | 0560 |
| | C-like dJ845O24.4 (hnRNP core protein | | | | |
| 469 | BRD4_HUMAN (O60885) Bromodomain-containing protein 4 (HUNK1 | O60885 | 152123.8 | 3.01E−04 | 0535 |
| | protein) | | | | |
| 470 | OPHN1_HUMAN (O60890) Oligophrenin 1 | O60890 | 91583.03 | 8.75E−03 | 0571 |
| 471 | O75137 (O75137) KIAA0642 protein (Fragment) | O75137 | 153399.6 | 4.21E−03 | 0535 |
| 472 | HIP1R_HUMAN (O75146) Huntingtin-interacting protein 1-related | O75146 | 119315 | 6.12E−03 | 0578 |
| | protein (Hip1-related) (Hip 12) | | | | |
| 473 | O75322 (O75322) Hsp89-alpha-delta-N | O75322 | 63211.7 | 1.24E−05 | 0578 |
| 474 | FNBP3_HUMAN (O75400) Formin-binding protein 3 (Huntingtin yeast | O75400 | 108736.4 | 2.50E−03 | 0571 |
| | partner A) (Huntingtin-interacting | | | | |
| 475 | CREG1_HUMAN (O75629) CREG1 protein precursor (Cellular | O75629 | 24059.31 | 1.02E−05 | 0541 |
| | repressor of E1A-stimulated genes 1) | | | | |
| 476 | OFD1_HUMAN (O75665) Oral-facial-digital syndrome 1 protein | 075665 | 116598.9 | 3.36E−04 | 0541 |
| | (Protein 71-7A) | | | | |
| 477 | O75703 (O75703) Hypothetical protein A-270G1.2 | O75703 | 12404.55 | 4.17E−03 | 0552 |
| 478 | TCEA3_HUMAN (O75764) Transcription elongation factor A protein 3 | O75764 | 38946.71 | 8.19E−03 | 0574 |
| | (Transcription elongation factor S | | | | |
| 479 | FTHFD_HUMAN (O75891) 10-formyltetrahydrofolate dehydrogenase | O75891 | 98766.77 | 8.40E−04 | 0549 |
| | (EC 1.5.1.6) (10-FTHFDH) (Aldehyde deh | | | | |
| 480 | ST1C2_HUMAN (O75897) Sulfotransferase 1C2 (EC 2.8.2.—) | O75897 | 35510.89 | 7.10E−03 | 0578 |
| | (SULT1C) (SULT1C#2) | | | | |
| 481 | SYUG_HUMAN (O76070) Gamma-synuclein (Persyn) (Breast cancer- | O76070 | 13292.8 | 1.54E−03 | 0560 |
| | specific gene 1 protein) (Synoretin) (S | | | | |
| 482 | DDAH1_HUMAN (O94760) NG,NG-dimethylarginine | O94760 | 30970.89 | 6.99E−03 | 0578 |
| | dimethylaminohydrolase 1 (EC 3.5.3.18) (Dimethylarginin | | | | |
| 483 | AT10B_HUMAN (O94823) Probable phospholipid-transporting | O94823 | 165283.6 | 1.82E−03 | 0541 |
| | ATPase VB (EC 3.6.3.1) | | | | |
| 484 | O94843 (O94843) KIAA0739 protein (Fragment) | O94843 | 127881.5 | 5.66E−03 | 0574 |
| 485 | NFASC_HUMAN (O94856) Neurofascin precursor | O94856 | 137533 | 1.38E−03 | 0541 |
| 486 | O94969 (O94969) KIAA0895 protein (Fragment) | O94969 | 61219.9 | 7.16E−03 | 0549 |
| 487 | UNC5C_HUMAN (O95185) Netrin receptor UNC5C precursor (Unc-5 | O95185 | 103036 | 5.25E−03 | 0541 |
| | homolog C) (Unc-5 homolog 3) | | | | |
| 488 | O95267 (O95267) Calcium and DAG-regulated guanine nucleotide | O95267 | 90272.02 | 6.48E−03 | 0541 |
| | exchange factor II | | | | |
| 489 | O95285 (O95285) Erythroblast macrophage protein EMP | O95285 | 43879.31 | 9.52E−03 | 0578 |
| 490 | SMC2_HUMAN (O95347) Structural maintenance of chromosome 2- | O95347 | 135695.7 | 4.58E−03 | 0578 |
| | like 1 protein (Chromosome-associated pr | | | | |
| 491 | ZBT7A_HUMAN (O95365) Zinc finger and BTB domain containing | O95365 | 61400.79 | 7.26E−03 | 0541 |
| | protein 7A (Leukemia/lymphoma related fa | | | | |
| 492 | ATS2_HUMAN (O95450) ADAMTS-2 precursor (EC 3.4.24.14) (A | O95450 | 134635.7 | 3.67E−03 | 0574 |
| | disintegrin and metalloproteinase with thr | | | | |
| 493 | HS74L_HUMAN (O95757) Heat shock 70 kDa protein 4L (Osmotic | O95757 | 94426.4 | 1.12E−03 | 0567 |
| | stress protein 94) (Heat shock 70-relate | | | | |
| 494 | STAU_HUMAN (O95793) Double-stranded RNA-binding protein | O95793 | 63227.92 | 6.38E−03 | 0552 |
| | Staufen homolog | | | | |
| 495 | UCP4_HUMAN (O95847) Mitochondrial uncoupling protein 4 (UCP 4) | O95847 | 36040.65 | 1.45E−03 | 0571 |
| | (Solute carrier family 25 member 27) | | | | |
| 496 | SODC_HUMAN (P00441) Superoxide dismutase [Cu—Zn] (EC | P00441 | 15794.87 | 4.16E−03 | 0541 |
| | 1.15.1.1) | | | | |
| 497 | C1R_HUMAN (P00736) Complement C1r subcomponent precursor | P00736 | 80121.9 | 3.35E−08 | 0541 |
| | (EC 3.4.21.41) (Complement component 1, r | | | | |
| 498 | CFAD_HUMAN (P00746) Complement factor D precursor (EC | P00746 | 26986.84 | 8.77E−05 | 0578 |
| | 3.4.21.46) (C3 convertase activator) (Properd | | | | |
| 499 | CAH1_HUMAN (P00915) Carbonic anhydrase 1 (EC 4.2.1.1) | P00915 | 28721.34 | 1.12E−07 | 0552 |
| | (Carbonic anhydrase I) (Carbonate dehydratase | | | | |
| 500 | CAH2_HUMAN (P00918) Carbonic anhydrase 2 (EC 4.2.1.1) | P00918 | 29096.89 | 6.62E−03 | 0560 |
| | (Carbonic anhydrase II) (Carbonate dehydratas | | | | |
| 501 | KV2C_HUMAN (P01616) Ig kappa chain V-II region MIL | P01616 | 12048.05 | 9.27E−06 | 0578 |
| 502 | KV3C_HUMAN (P01621) Ig kappa chain V-III region NG9 precursor | P01621 | 10722.28 | 9.08E−03 | 0578 |
| | (Fragment) | | | | |
| 503 | HV1A_HUMAN (P01742) Ig heavy chain V-I region EU | P01742 | 12464.08 | 8.83E−03 | 0556 |
| 504 | HV3B_HUMAN (P01763) Ig heavy chain V-III region WEA | P01763 | 12248.52 | 7.67E−04 | 0535 |
| 505 | HV3C_HUMAN (P01764) Ig heavy chain V-III region VH26 precursor | P01764 | 12574.22 | 5.13E−03 | 0556 |
| 506 | HV3G_HUMAN (P01768) Ig heavy chain V-III region CAM | P01768 | 13659.17 | 4.59E−05 | 0535 |
| 507 | HV3J_HUMAN (P01771) Ig heavy chain V-III region HIL | P01771 | 13557.22 | 3.79E−03 | 0535 |
| 508 | HV3T_HUMAN (P01781) Ig heavy chain V-III region GAL | P01781 | 12722.21 | 9.93E−03 | 0535 |
| 509 | APOC3_HUMAN (P02656) Apolipoprotein C-III precursor (Apo-CIII) | P02656 | 10845.5 | 5.51E−05 | 0552 |
| | (ApoC-III) | | | | |
| 510 | C1QB_HUMAN (P02746) Complement C1q subcomponent, B chain | P02746 | 26442.4 | 2.43E−06 | 0578 |
| | precursor | | | | |
| 511 | FINC_HUMAN (P02751) Fibronectin precursor (FN) (Cold-insoluble | P02751 | 262439.5 | 2.14E−03 | 0560 |
| | globulin) (CIG) | | | | |
| 512 | ESR1_HUMAN (P03372) Estrogen receptor (ER) (Estradiol receptor) | P03372 | 66173.23 | 1.85E−03 | 0560 |
| | (ER-alpha) | | | | |
| 513 | ALK1_HUMAN (P03973) Antileukoproteinase 1 precursor (ALP) | P03973 | 14315.91 | 6.02E−03 | 0571 |
| | (HUSI-1) (Seminal proteinase inhibitor) ( | | | | |
| 514 | HMDH_HUMAN (P04035) 3-hydroxy-3-methylglutaryl-coenzyme A | P04035 | 97412.74 | 2.11E−03 | 0556 |
| | reductase (EC 1.1.1.34) (HMG-CoA reductas | | | | |
| 515 | MT1A_HUMAN (P04731) Metallothionein-1A (MT-1A) | P04731 | 6128.206 | 1.40E−05 | 0560 |
| | (Metallothionein-IA) (MT-IA) | | | | |
| 516 | MT1F_HUMAN (P04733) Metallothionein-1F (MT-1F) | P04733 | 6081.223 | 8.20E−06 | 0560 |
| | (Metallothionein-IF) (MT-IF) | | | | |
| 517 | APOD_HUMAN (P05090) Apolipoprotein D precursor (Apo-D) (ApoD) | P05090 | 21261.77 | 4.72E−03 | 0552 |
| 518 | INSR_HUMAN (P06213) Insulin receptor precursor (EC 2.7.1.112) | P06213 | 156206.1 | 1.01E−03 | 0560 |
| | (IR) (CD220 antigen) [Contains: Insul | | | | |
| 519 | KV40_HUMAN (P06312) Ig kappa chain V-IV region precursor | P06312 | 13371.6 | 6.14E−05 | 0556 |
| | (Fragment) | | | | |
| 520 | S10A9_HUMAN (P06702) Calgranulin B (Migration inhibitory factor- | P06702 | 13233.5 | 6.68E−06 | 0571 |
| | related protein 14) (MRP-14) (P14) | | | | |
| 521 | G6PI_HUMAN (P06744) Glucose-6-phosphate isomerase (EC | P06744 | 62976.29 | 1.26E−03 | 0578 |
| | 5.3.1.9) (GPI) (Phosphoglucose isomerase) (PG | | | | |
| 522 | ADHA_HUMAN (P07327) Alcohol dehydrogenase alpha chain (EC | P07327 | 39701.49 | 7.62E−07 | 0560 |
| | 1.1.1.1) | | | | |
| 523 | OPSD_HUMAN (P08100) Rhodopsin (Opsin 2) | P08100 | 38866.51 | 3.01E−09 | 0574 |
| 524 | SODE_HUMAN (P08294) Extracellular superoxide dismutase [Cu—Zn] | P08294 | 25864.7 | 6.83E−07 | 0541 |
| | precursor (EC 1.15.1.1) (EC-SOD) | | | | |
| 525 | DOPO_HUMAN (P09172) Dopamine beta-hydroxylase precursor (EC | P09172 | 67570.06 | 3.69E−03 | 0574 |
| | 1.14.17.1) (Dopamine beta-monooxygenase | | | | |
| 526 | PARP1_HUMAN (P09874) Poly [ADP-ribose] polymerase 1 (EC | P09874 | 112881.4 | 5.02E−03 | 0574 |
| | 2.4.2.30) (PARP-1) (ADPRT) (NAD(+) ADP-ribo | | | | |
| 527 | UCHL1_HUMAN (P09936) Ubiquitin carboxyl-terminal hydrolase | P09936 | 24808.46 | 8.46E−05 | 0560 |
| | isozyme L1 (EC 3.4.19.12) (EC 6.—.—.—) ( | | | | |
| 528 | ALDOC_HUMAN (P09972) Fructose-bisphosphate aldolase C (EC | P09972 | 39300.21 | 2.20E−05 | 0578 |
| | 4.1.2.13) (Brain-type aldolase) | | | | |
| 529 | TAU_HUMAN (P10636) Microtubule-associated protein tau | P10636 | 78698.83 | 9.98E−03 | 0535 |
| | (Neurofibrillary tangle protein) (Paired heli | | | | |
| 530 | CO7_HUMAN (P10643) Complement component C7 precursor | P10643 | 93457.27 | 6.15E−04 | 0552 |
| 531 | PHS3_HUMAN (P11216) Glycogen phosphorylase, brain form (EC | P11216 | 96503.63 | 2.15E−03 | 0552 |
| | 2.4.1.1) | | | | |
| 532 | K2C3_HUMAN (P12035) Keratin, type II cytoskeletal 3 (Cytokeratin-3) | P12035 | 64464.63 | 1.41E−05 | 0552 |
| | (CK-3) (Keratin-3) (K3) (65 kDa | | | | |
| 533 | CO6A1_HUMAN (P12109) Collagen alpha 1(VI) chain precursor | P12109 | 108480.1 | 7.42E−06 | 0567 |
| 534 | IMDH2_HUMAN (P12268) Inosine-5′-monophosphate dehydrogenase | P12268 | 55769.72 | 7.59E−03 | 0571 |
| | 2 (EC 1.1.1.205) (IMP dehydrogenase 2) | | | | |
| 535 | CRAL_HUMAN (P12271) Cellular retinaldehyde-binding protein | P12271 | 36320.25 | 5.43E−08 | 0560 |
| | (CRALBP) | | | | |
| 536 | ANXA3_HUMAN (P12429) Annexin A3 (Annexin III) (Lipocortin III) | P12429 | 36221.62 | 8.89E−03 | 0560 |
| | (Placental anticoagulant protein III | | | | |
| 537 | K2C5_HUMAN (P13647) Keratin, type II cytoskeletal 5 (Cytokeratin-5) | P13647 | 62409.08 | 6.24E−04 | 0578 |
| | (CK-5) (Keratin-5) (K5) (58 kDa | | | | |
| 538 | MTDC_HUMAN (P13995) Bifunctional methylenetetrahydrofolate | P13995 | 37296.94 | 4.32E−03 | 0541 |
| | dehydrogenase/cyclohydrolase, mitochondr | | | | |
| 539 | IL1R1_HUMAN (P14778) Interleukin-1 receptor type I precursor (IL- | P14778 | 65360.72 | 9.77E−03 | 0549 |
| | 1R-1) (IL-1RT1) (IL-1R-alpha) (p80 | | | | |
| 540 | CD19_HUMAN (P15391) B-lymphocyte antigen CD19 precursor (B- | P15391 | 61030.84 | 6.70E−03 | 0574 |
| | lymphocyte surface antigen B4) (Leu-12) | | | | |
| 541 | PLCG2_HUMAN (P16885) 1-phosphatidylinositol-4,5-bisphosphate | P16885 | 147842.9 | 7.79E−03 | 0549 |
| | phosphodiesterase gamma 2 (EC 3.1.4.11 | | | | |
| 542 | ZNF20_HUMAN (P17024) Zinc finger protein 20 (Zinc finger protein | P17024 | 61527.24 | 5.06E−04 | 0541 |
| | KOX13) (DKFZp572P0920) | | | | |
| 543 | AK1C4_HUMAN (P17516) Aldo-keto reductase family 1 member C4 | P17516 | 37071.01 | 6.23E−03 | 0571 |
| | (EC 1.1.1.—) (Chlordecone reductase) (E | | | | |
| 544 | DESM_HUMAN (P17661) Desmin | P17661 | 53372.19 | 2.95E−03 | 0560 |
| 545 | ITB6_HUMAN (P18564) Integrin beta-6 precursor | P18564 | 85879.23 | 9.63E−03 | 0535 |
| 546 | SDC1_HUMAN (P18827) Syndecan-1 precursor (SYND1) (CD138 | P18827 | 32456.83 | 6.98E−03 | 0556 |
| | antigen) | | | | |
| 547 | AMD_HUMAN (P19021) Peptidyl-glycine alpha-amidating | P19021 | 108263.4 | 1.42E−03 | 0541 |
| | monooxygenase precursor (EC 1.14.17.3) (PAM) | | | | |
| 548 | CADH2_HUMAN (P19022) Neural-cadherin precursor (N-cadherin) | P19022 | 99789.42 | 2.80E−04 | 0556 |
| | (Cadherin-2) | | | | |
| 549 | PLCG1_HUMAN (P19174) 1-phosphatidylinositol-4,5-bisphosphate | P19174 | 148437.5 | 2.40E−03 | 0549 |
| | phosphodiesterase gamma 1 (EC 3.1.4.11 | | | | |
| 550 | ITIH2_HUMAN (P19823) Inter-alpha-trypsin inhibitor heavy chain H2 | P19823 | 106369.8 | 6.05E−06 | 0552 |
| | precursor (ITI heavy chain H2) (I | | | | |
| 551 | HXA5_HUMAN (P20719) Homeobox protein Hox-A5 (Hox-1C) | P20719 | 29327 | 2.97E−03 | 0552 |
| 552 | PTN_HUMAN (P21246) Pleiotrophin precursor (PTN) (Heparin- | P21246 | 18929.67 | 6.84E−04 | 0567 |
| | binding growth-associated molecule) (HB-GA | | | | |
| 553 | CALB2_HUMAN (P22676) Calretinin (CR) (29 kDa calbindin) | P22676 | 31501.63 | 7.50E−05 | 0578 |
| 554 | COF1_HUMAN (P23528) Cofilin-1 (Cofilin, non-muscle isoform) (18 kDa | P23528 | 18359.62 | 6.52E−06 | 0578 |
| | phosphoprotein) (p18) | | | | |
| 555 | THTM_HUMAN (P25325) 3-mercaptopyruvate sulfurtransferase (EC | P25325 | 33026.6 | 8.45E−03 | 0560 |
| | 2.8.1.2) (MST) | | | | |
| 556 | ATPA_HUMAN (P25705) ATP synthase alpha chain, mitochondrial | P25705 | 59713.73 | 5.75E−04 | 0578 |
| | precursor (EC 3.6.3.14) | | | | |
| 557 | CO8A1_HUMAN (P27658) Collagen alpha 1(VIII) chain precursor | P27658 | 73317.22 | 4.71E−03 | 0578 |
| | (Endothelial collagen) | | | | |
| 558 | GSTM2_HUMAN (P28161) Glutathione S-transferase Mu 2 (EC | P28161 | 25596.95 | 9.81E−05 | 0560 |
| | 2.5.1.18) (GSTM2-2) (GST class-mu 2) | | | | |
| 559 | PRDX6_HUMAN (P30041) Peroxiredoxin 6 (EC 1.11.1.15) | P30041 | 24888.15 | 9.95E−05 | 0560 |
| | (Antioxidant protein 2) (1-Cys peroxiredoxin) ( | | | | |
| 560 | ILEU_HUMAN (P30740) Leukocyte elastase inhibitor (LEI) (Serpin | P30740 | 42714.77 | 2.07E−03 | 0571 |
| | B1) (Monocyte/neutrophil elastase in | | | | |
| 561 | GDIA_HUMAN (P31150) Rab GDP dissociation inhibitor alpha (Rab | P31150 | 50550.21 | 2.15E−09 | 0578 |
| | GDI alpha) (GDI-1) (XAP-4) (Oligophre | | | | |
| 562 | PRDX2_HUMAN (P32119) Peroxiredoxin 2 (EC 1.11.1.15) | P32119 | 21747.2 | 9.20E−03 | 0552 |
| | (Thioredoxin peroxidase 1) (Thioredoxin-depende | | | | |
| 563 | MCM5_HUMAN (P33992) DNA replication licensing factor MCM5 | P33992 | 82233.27 | 2.80E−03 | 0578 |
| | (CDC46 homolog) (P1-CDC46) | | | | |
| 564 | TSP2_HUMAN (P35442) Thrombospondin-2 precursor | P35442 | 129871.5 | 7.68E−03 | 0552 |
| 565 | TSP4_HUMAN (P35443) Thrombospondin-4 precursor | P35443 | 105801.8 | 7.50E−03 | 0535 |
| 566 | PPCKC_HUMAN (P35558) Phosphoenolpyruvate carboxykinase, | P35558 | 69163.79 | 4.40E−03 | 0541 |
| | cytosolic [GTP] (EC 4.1.1.32) (Phosphoenolp | | | | |
| 567 | IRS1_HUMAN (P35568) Insulin receptor substrate 1 (IRS-1) | P35568 | 131509 | 7.44E−03 | 0541 |
| 568 | MYH11_HUMAN (P35749) Myosin-11 (Myosin heavy chain, smooth | P35749 | 227197.9 | 1.79E−03 | 0560 |
| | muscle isoform) (SMMHC) | | | | |
| 569 | GLRX1_HUMAN (P35754) Glutaredoxin-1 (Thioltransferase-1) | P35754 | 11637.14 | 9.41E−06 | 0560 |
| | (TTase-1) | | | | |
| 570 | ZNF93_HUMAN (P35789) Zinc finger protein 93 (Zinc finger protein | P35789 | 70526.29 | 8.78E−05 | 0535 |
| | HTF34) (Fragment) | | | | |
| 571 | MOT8_HUMAN (P36021) Monocarboxylate transporter 8 (MCT 8) (X- | P36021 | 66357.57 | 5.94E−03 | 0567 |
| | linked PEST-containing transporter) (M | | | | |
| 572 | PERI_HUMAN (P41219) Peripherin | P41219 | 53845.66 | 8.63E−05 | 0560 |
| 573 | JAD1C_HUMAN (P41229) Jumonji/ARID domain-containing protein | P41229 | 175692.8 | 3.07E−04 | 0571 |
| | 1C (SmcX protein) (Xe169 protein) | | | | |
| 574 | SK2L2_HUMAN (P42285) Superkiller viralicidic activity 2-like 2 | P42285 | 117729.4 | 2.00E−05 | 0556 |
| 575 | TEC_HUMAN (P42680) Tyrosine-protein kinase Tec (EC 2.7.1.112) | P42680 | 73581.92 | 9.43E−03 | 0556 |
| 576 | DCC_HUMAN (P43146) Netrin receptor DCC precursor (Tumor | P43146 | 158356.8 | 2.63E−03 | 0541 |
| | suppressor protein DCC) (Colorectal cancer | | | | |
| 577 | MATR3_HUMAN (P43243) Matrin-3 | P43243 | 94564.67 | 2.10E−03 | 0552 |
| 578 | RANG_HUMAN (P43487) Ran-specific GTPase-activating protein | P43487 | 23295.58 | 8.40E−03 | 0552 |
| | (Ran binding protein 1) (RanBP1) | | | | |
| 579 | AFAM_HUMAN (P43652) Afamin precursor (Alpha-albumin) (Alpha- | P43652 | 69024.09 | 3.85E−07 | 0549 |
| | Alb) | | | | |
| 580 | S6A12_HUMAN (P48065) Sodium- and chloride-dependent betaine | P48065 | 69382.38 | 9.89E−04 | 0541 |
| | transporter (Na+/Cl-betaine/GABA transp | | | | |
| 581 | PCY1A_HUMAN (P49585) Choline-phosphate cytidylyltransferase A | P49585 | 41705.8 | 6.56E−03 | 0549 |
| | (EC 2.7.7.15) (Phosphorylcholine tran | | | | |
| 582 | PPM1F_HUMAN (P49593) Ca(2+)/calmodulin-dependent protein | P49593 | 49800.19 | 4.81E−03 | 0567 |
| | kinase phosphatase (EC 3.1.3.16) (CaM-kina | | | | |
| 583 | NUMB_HUMAN (P49757) Protein numb homolog (h-Numb) (Protein | P49757 | 70759.55 | 2.24E−03 | 0549 |
| | S171) | | | | |
| 584 | ST1E1_HUMAN (P49888) Estrogen sulfotransferase (EC 2.8.2.4) | P49888 | 35103.59 | 9.33E−03 | 0552 |
| | (Sulfotransferase, estrogen-preferring) | | | | |
| 585 | IDH3A_HUMAN (P50213) Isocitrate dehydrogenase [NAD] subunit | P50213 | 39566.1 | 6.27E−05 | 0552 |
| | alpha, mitochondrial precursor (EC 1.1. | | | | |
| 586 | PRELP_HUMAN (P51888) Prolargin precursor (Proline-arginine-rich | P51888 | 43782.22 | 3.39E−03 | 0578 |
| | end leucine-rich repeat protein) | | | | |
| 587 | NUBP1_HUMAN (P53384) Nucleotide-binding protein 1 (NBP 1) | P53384 | 34566.41 | 9.03E−03 | 0552 |
| 588 | COPB_HUMAN (P53618) Coatomer beta subunit (Beta-coat protein) | P53618 | 107070.8 | 6.87E−04 | 0578 |
| | (Beta-COP) | | | | |
| 589 | COPA_HUMAN (P53621) Coatomer alpha subunit (Alpha-coat | P53621 | 138243.8 | 5.80E−03 | 0571 |
| | protein) (Alpha-COP) (HEPCOP) (HEP-COP) [Con | | | | |
| 590 | OAZ1_HUMAN (P54368) Ornithine decarboxylase antizyme (ODC- | P54368 | 25258.65 | 5.07E−04 | 0552 |
| | Az) | | | | |
| 591 | RD23A_HUMAN (P54725) UV excision repair protein RAD23 homolog | P54725 | 39584.69 | 6.23E−03 | 0574 |
| | A (hHR23A) | | | | |
| 592 | CAD11_HUMAN (P55287) Cadherin-11 precursor (Osteoblast- | P55287 | 87994.91 | 3.30E−03 | 0578 |
| | cadherin) (OB-cadherin) (OSF-4) | | | | |
| 593 | ITA1_HUMAN (P56199) Integrin alpha-1 (Laminin and collagen | P56199 | 127757.1 | 4.11E−03 | 0560 |
| | receptor) (VLA-1) (CD49a) | | | | |
| 594 | ANTR2_HUMAN (P58335) Anthrax toxin receptor 2 precursor | P58335 | 53658.2 | 6.76E−03 | 0567 |
| | (Capillary morphogenesis gene 2 protein) (C | | | | |
| 595 | USH3A_HUMAN (P58418) Usher syndrome type 3 protein | P58418 | 13412.01 | 4.16E−03 | 0549 |
| 596 | NAL12_HUMAN (P59046) NACHT-, LRR- and PYD-containing protein | P59046 | 120095.3 | 7.26E−05 | 0552 |
| | 12 (PYRIN-containing APAF1-like protei | | | | |
| 597 | RAB15_HUMAN (P59190) Ras-related protein Rab-15 | P59190 | 24375.19 | 8.65E−03 | 0535 |
| 598 | SNP25_HUMAN (P60880) Synaptosomal-associated protein 25 | P60880 | 23300.21 | 8.96E−04 | 0571 |
| | (SNAP-25) (Synaptosomal-associated 25 kDa p | | | | |
| 599 | PSME3_HUMAN (P61289) Proteasome activator complex subunit 3 | P61289 | 29487.55 | 4.58E−03 | 0578 |
| | (Proteasome activator 28-gamma subunit) | | | | |
| 600 | HNRPK_HUMAN (P61978) Heterogeneous nuclear ribonucleoprotein | P61978 | 50944.43 | 5.90E−06 | 0578 |
| | K (hnRNP K) (Transformation up-regulat | | | | |
| 601 | NCS1_HUMAN (P62166) Neuronal calcium sensor 1 (NCS-1) | P62166 | 21733.87 | 2.01E−03 | 0535 |
| | (Frequenin homolog) (Frequenin-like protein) | | | | |
| 602 | 1433E_HUMAN (P62258) 14-3-3 protein epsilon (14-3-3E) | P62258 | 29155.42 | 5.71E−06 | 0560 |
| 603 | GBB1_HUMAN (P62873) Guanine nucleotide-binding protein | P62873 | 37221.98 | 9.16E−04 | 0574 |
| | G(I)/G(S)/G(T) beta subunit 1 (Transducin be | | | | |
| 604 | IF4G2_HUMAN (P78344) Eukaryotic translation initiation factor 4 | P78344 | 102296.8 | 8.98E−03 | 0552 |
| | gamma 2 (eIF-4-gamma 2) (eIF-4G 2) | | | | |
| 605 | IRX4_HUMAN (P78413) Iroquois-class homeodomain protein IRX-4 | P78413 | 54411.1 | 9.00E−03 | 0552 |
| | (Iroquois homeobox protein 4) (Homeodo | | | | |
| 606 | M3K9_HUMAN (P80192) Mitogen-activated protein kinase kinase | P80192 | 121788.7 | 8.54E−04 | 0574 |
| | kinase 9 (EC 2.7.1.37) (Mixed lineage k | | | | |
| 607 | TSG6_HUMAN (P98066) Tumor necrosis factor-inducible protein | P98066 | 31183.3 | 5.22E−03 | 0552 |
| | TSG-6 precursor (TNF-stimulated gene 6 | | | | |
| 608 | LRP2_HUMAN (P98164) Low-density lipoprotein receptor-related | P98164 | 521587.6 | 1.10E−03 | 0541 |
| | protein 2 precursor (Megalin) (Glycopr | | | | |
| 609 | K22O_HUMAN (Q01546) Keratin, type II cytoskeletal 2 oral | Q01546 | 65830.09 | 7.49E−03 | 0574 |
| | (Cytokeratin-2P) (K2P) (CK 2P) | | | | |
| 610 | KIF23_HUMAN (Q02241) Kinesin-like protein KIF23 (Mitotic kinesin- | Q02241 | 98043.87 | 6.25E−03 | 0578 |
| | like protein 1) (Kinesin-like prot | | | | |
| 611 | GNA12_HUMAN (Q03113) Guanine nucleotide-binding protein, alpha- | Q03113 | 44120.41 | 4.17E−03 | 0567 |
| | 12 subunit (G alpha 12) | | | | |
| 612 | ERCC6_HUMAN (Q03468) DNA excision repair protein ERCC-6 | Q03468 | 168310.9 | 7.25E−03 | 0571 |
| | (Cockayne syndrome protein CSB) | | | | |
| 613 | FHR1_HUMAN (Q03591) Complement factor H-related protein 1 | Q03591 | 37636.97 | 6.53E−08 | 0535 |
| | precursor (FHR-1) (H factor-like protein | | | | |
| 614 | K1C17_HUMAN (Q04695) Keratin, type I cytoskeletal 17 (Cytokeratin- | Q04695 | 47945.07 | 8.27E−04 | 0578 |
| | 17) (CK-17) (Keratin-17) (K17) (3 | | | | |
| 615 | AK1C1_HUMAN (Q04828) Aldo-keto reductase family 1 member C1 | Q04828 | 36765.05 | 7.32E−03 | 0560 |
| | (EC 1.1.1.—) (20-alpha-hydroxysteroid d | | | | |
| 616 | 1433F_HUMAN (Q04917) 14-3-3 protein eta (Protein AS1) | Q04917 | 28069.98 | 5.34E−03 | 0578 |
| 617 | PTN12_HUMAN (Q05209) Tyrosine-protein phosphatase, non- | Q05209 | 88065.49 | 6.26E−03 | 0556 |
| | receptor type 12 (EC 3.1.3.48) (Protein-tyro | | | | |
| 618 | KPCZ_HUMAN (Q05513) Protein kinase C, zeta type (EC 2.7.1.37) | Q05513 | 67616.83 | 8.99E−05 | 0567 |
| | (nPKC-zeta) | | | | |
| 619 | AMPE_HUMAN (Q07075) Glutamyl aminopeptidase (EC 3.4.11.7) | Q07075 | 109175.6 | 6.99E−03 | 0552 |
| | (EAP) (Aminopeptidase A) (APA) (Different | | | | |
| 620 | Q07900 (Q07900) M130 antigen cytoplasmic variant 2 precursor | Q07900 | 125271.2 | 1.61E−05 | 0541 |
| 621 | Q08357 (Q08357) Leukemia virus receptor 2 (Solute carrier family 20, | Q08357 | 70347.52 | 8.11E−03 | 0556 |
| | member 2) | | | | |
| 622 | MFGM_HUMAN (Q08431) Lactadherin precursor (Milk fat globule- | Q08431 | 43095.49 | 7.96E−04 | 0567 |
| | EGF factor 8) (MFG-E8) (HMFG) (Breast e | | | | |
| 623 | KIF1A_HUMAN (Q12756) Kinesin-like protein KIF1A (Axonal | Q12756 | 190963.1 | 4.17E−03 | 0567 |
| | transporter of synaptic vesicles) | | | | |
| 624 | CHD3_HUMAN (Q12873) Chromodomain helicase-DNA-binding | Q12873 | 220552.2 | 8.41E−03 | 0556 |
| | protein 3 (CHD-3) (Mi-2 autoantigen 240 kDa p | | | | |
| 625 | SEPR_HUMAN (Q12884) Seprase (EC 3.4.21.—) (Fibroblast activation | Q12884 | 87764.87 | 4.65E−03 | 0541 |
| | protein alpha) (Integral membrane | | | | |
| 626 | PTN13_HUMAN (Q12923) Tyrosine-protein phosphatase, non- | Q12923 | 276731.3 | 2.13E−03 | 0578 |
| | receptor type 13 (EC 3.1.3.48) (Protein-tyro | | | | |
| 627 | NFX1_HUMAN (Q12986) Transcriptional repressor NF-X1 (EC 6.3.2.—) | Q12986 | 123067.8 | 9.18E−03 | 0571 |
| | (Nuclear transcription factor, X b | | | | |
| 628 | AKAP6_HUMAN (Q13023) A-kinase anchor protein 6 (Protein kinase | Q13023 | 256503.6 | 1.24E−03 | 0578 |
| | A anchoring protein 6) (PRKA6) (A-ki | | | | |
| 629 | NMDE2_HUMAN (Q13224) Glutamate [NMDA] receptor subunit | Q13224 | 166260.7 | 4.73E−03 | 0571 |
| | epsilon 2 precursor (N-methyl D-aspartate re | | | | |
| 630 | ATM_HUMAN (Q13315) Serine-protein kinase ATM (EC 2.7.1.37) | Q13315 | 350417.1 | 7.00E−03 | 0552 |
| | (Ataxia telangiectasia mutated) (A-T, mu | | | | |
| 631 | LSAMP_HUMAN (Q13449) Limbic system-associated membrane | Q13449 | 37285.17 | 3.50E−06 | 0567 |
| | protein precursor (LSAMP) | | | | |
| 632 | MYO9B_HUMAN (Q13459) Myosin-9B (Myosin IXb) (Unconventional | Q13459 | 243404.2 | 3.54E−03 | 0541 |
| | myosin-9b) | | | | |
| 633 | SNX1_HUMAN (Q13596) Sorting nexin-1 | Q13596 | 59033.21 | 1.83E−03 | 0578 |
| 634 | HRB2_HUMAN (Q13601) HIV-1 Rev-binding protein 2 (Rev- | Q13601 | 43506.66 | 5.33E−03 | 0549 |
| | interacting protein 1) (Rip-1) | | | | |
| 635 | Q13715 (Q13715) Hypothetical protein | Q13715 | 26694.33 | 1.29E−03 | 0549 |
| 636 | Q13876 (Q13876) Bone-derived growth factor (Fragment) | Q13876 | 86341.05 | 4.51E−03 | 0567 |
| 637 | PEBB_HUMAN (Q13951) Core-binding factor, beta subunit (CBF- | Q13951 | 21494.7 | 2.21E−03 | 0541 |
| | beta) (Polyomavirus enhancer binding pro | | | | |
| 638 | K0153_HUMAN (Q14166) Protein KIAA0153 | Q14166 | 74356.21 | 3.83E−03 | 0567 |
| 639 | DOCK1_HUMAN (Q14185) Dedicator of cytokinesis protein 1 (180 kDa | Q14185 | 215237.7 | 6.65E−03 | 0578 |
| | protein downstream of CRK) (DOCK18 | | | | |
| 640 | BTG3_HUMAN (Q14201) BTG3 protein (Tob5 protein) (Abundant in | Q14201 | 29097.7 | 7.78E−03 | 0578 |
| | neuroepithelium area protein) | | | | |
| 641 | SMRA3_HUMAN (Q14527) SWI/SNF-related matrix-associated actin- | Q14527 | 113856.7 | 2.41E−03 | 0560 |
| | dependent regulator of chromatin subfa | | | | |
| 642 | DSC3_HUMAN (Q14574) Desmocollin-3 precursor (Desmocollin-4) | Q14574 | 99950.31 | 6.01E−03 | 0552 |
| | (HT-CP) | | | | |
| 643 | SMC1A_HUMAN (Q14683) Structural maintenance of chromosome 1- | Q14683 | 143143.6 | 4.09E−03 | 0549 |
| | like 1 protein (SMC1alpha protein) (DXS | | | | |
| 644 | NUMA1_HUMAN (Q14980) Nuclear mitotic apparatus protein 1 | Q14980 | 238128.2 | 8.47E−03 | 0552 |
| | (NuMA protein) (SP-H antigen) | | | | |
| 645 | Q15015 (Q15015) KIAA0155 protein (Fragment) | Q15015 | 135595.6 | 6.25E−03 | 0549 |
| 646 | PLCB4_HUMAN (Q15147) 1-phosphatidylinositol-4,5-bisphosphate | Q15147 | 134377.8 | 6.87E−03 | 0560 |
| | phosphodiesterase beta 4 (EC 3.1.4.11) | | | | |
| 647 | PTPRK_HUMAN (Q15262) Receptor-type tyrosine-protein | Q15262 | 161984.1 | 1.51E−03 | 0552 |
| | phosphatase kappa precursor (EC 3.1.3.48) (Prot | | | | |
| 648 | ERBB4_HUMAN (Q15303) Receptor tyrosine-protein kinase erbB-4 | Q15303 | 146712.7 | 9.26E−03 | 0560 |
| | precursor (EC 2.7.1.112) (p180erbB4) ( | | | | |
| 649 | Q15464 (Q15464) Shb | Q15464 | 64485.34 | 4.62E−03 | 0552 |
| 650 | TAF7_HUMAN (Q15545) Transcription initiation factor TFIID subunit 7 | Q15545 | 40234.48 | 8.84E−04 | 0556 |
| | (Transcription initiation facto | | | | |
| 651 | PTN14_HUMAN (Q15678) Tyrosine-protein phosphatase, non- | Q15678 | 135153.4 | 9.35E−04 | 0567 |
| | receptor type 14 (EC 3.1.3.48) (Protein-tyro | | | | |
| 652 | MYLK_HUMAN (Q15746) Myosin light chain kinase, smooth muscle | Q15746 | 210640.8 | 7.59E−03 | 0556 |
| | and non-muscle isozymes (EC 2.7.1.117) | | | | |
| 653 | Q15751 (Q15751) P532 | Q15751 | 531849.4 | 5.49E−03 | 0541 |
| 654 | NCOA1_HUMAN (Q15788) Nuclear receptor coactivator 1 (EC | Q15788 | 156641.7 | 9.06E−03 | 0556 |
| | 2.3.1.48) (NCoA-1) (Steroid receptor coacti | | | | |
| 655 | RK_HUMAN (Q15835) Rhodopsin kinase (EC 2.7.1.125) (RK) (G | Q15835 | 63485.22 | 9.90E−03 | 0541 |
| | protein-coupled receptor kinase 1) | | | | |
| 656 | NTRK3_HUMAN (Q16288) NT-3 growth factor receptor precursor (EC | Q16288 | 94394.73 | 7.95E−03 | 0552 |
| | 2.7.1.112) (Neurotrophic tyrosine ki | | | | |
| 657 | AINX_HUMAN (Q16352) Alpha-internexin (Alpha-Inx) (66 kDa | Q16352 | 55357.48 | 9.91E−06 | 0560 |
| | neurofilament protein) (Neurofilament-66) | | | | |
| 658 | DPYL2_HUMAN (Q16555) Dihydropyrimidinase-related protein 2 | Q16555 | 62254.68 | 4.35E−03 | 0578 |
| | (DRP-2) (Collapsin response mediator pro | | | | |
| 659 | KCC4_HUMAN (Q16566) Calcium/calmodulin-dependent protein | Q16566 | 51892.92 | 6.14E−03 | 0541 |
| | kinase type IV (EC 2.7.1.123) (CAM kinase- | | | | |
| 660 | QPCT_HUMAN (Q16769) Glutaminyl-peptide cyclotransferase | Q16769 | 40850.86 | 1.50E−05 | 0574 |
| | precursor (EC 2.3.2.5) (QC) (Glutaminyl-tRN | | | | |
| 661 | DDR2_HUMAN (Q16832) Discoidin domain receptor 2 precursor (EC | Q16832 | 96689.9 | 7.92E−03 | 0556 |
| | 2.7.1.112) (Receptor protein-tyrosine | | | | |
| 662 | Q30217 (Q30217) MHC class II HLA-DRB9 (Fragment) | Q30217 | 10482.02 | 3.95E−03 | 0571 |
| 663 | Q3Y8G9 (Q3Y8G9) Shank2E (Fragment) | Q3Y8G9 | 78504.73 | 2.27E−03 | 0552 |
| 664 | Q4G0R3 (Q4G0R3) Zinc finger protein 553 | Q4G0R3 | 67736.06 | 5.90E−03 | 0549 |
| 665 | Q4VC04 (Q4VC04) Hypothetical protein | Q4VC04 | 32092.91 | 1.44E−03 | 0574 |
| 666 | Q4VC34 (Q4VC34) Hypothetical protein LOC285331 | Q4VC34 | 96569.61 | 3.20E−03 | 0574 |
| 667 | Q503A8 (Q503A8) H2A histone family, member Y, isoform 3 | Q503A8 | 39520.48 | 1.92E−06 | 0560 |
| 668 | Q52LF0 (Q52LF0) Hypothetical protein KIAA1604 | Q52LF0 | 105470.4 | 3.01E−03 | 0556 |
| 669 | Q53ET0 (Q53ET0) Hypothetical protein (Fragment) | Q53ET0 | 73246.46 | 6.72E−03 | 0560 |
| 670 | Q53FI7 (Q53FI7) Four and a half LIM domains 1 variant (Fragment) | Q53FI7 | 31873.79 | 8.89E−03 | 0560 |
| 671 | Q53FS5 (Q53FS5) Processing of 5, ribonuclease P/MRP subunit | Q53FS5 | 18827.56 | 6.09E−03 | 0567 |
| | isoform a variant (Fragment) | | | | |
| 672 | Q53GL8 (Q53GL8) Hypothetical protein (Fragment) | Q53GL8 | 50985.3 | 6.34E−03 | 0571 |
| 673 | Q53HB9 (Q53HB9) DEAD (Asp-Glu-Ala-Asp) box polypeptide 56 | Q53HB9 | 61527.04 | 7.24E−03 | 0574 |
| | variant (Fragment) | | | | |
| 674 | Q562R4 (Q562R4) Actin-like protein (Fragment) | Q562R4 | 11602.93 | 7.88E−03 | 0541 |
| 675 | Q58FG0 (Q58FG0) Heat shock protein 90Ae | Q58FG0 | 38713.81 | 5.09E−03 | 0560 |
| 676 | Q59EM4 (Q59EM4) Dipeptidyl-peptidase II variant (Fragment) | Q59EM4 | 41299 | 1.20E−06 | 0541 |
| 677 | Q59F14 (Q59F14) Neutrophil cytosol factor 2 variant (Fragment) | Q59F14 | 67545.52 | 1.67E−03 | 0567 |
| 678 | Q59FE2 (Q59FE2) Ketohexokinase isoform a variant (Fragment) | Q59FE2 | 22233.73 | 3.08E−03 | 0549 |
| 679 | Q59FF3 (Q59FF3) RUN and FYVE domain-containing 1 variant | Q59FF3 | 35416.27 | 9.36E−03 | 0552 |
| | (Fragment) | | | | |
| 680 | Q59FH8 (Q59FH8) Diaphanous 1 variant (Fragment) | Q59FH8 | 143694.7 | 3.46E−03 | 0560 |
| 681 | Q59FM2 (Q59FM2) PWP2 periodic tryptophan protein homolog | Q59FM2 | 33469.2 | 2.68E−03 | 0552 |
| | (Fragment) | | | | |
| 682 | Q59H20 (Q59H20) Hypothetical protein hypothetical protein FLJ21019 | Q59H20 | 84815.45 | 8.36E−03 | 0571 |
| | variant (Fragment) | | | | |
| 683 | Q5FBB4 (Q5FBB4) Shugoshin-like protein 1, transcript variant C1 | Q5FBB4 | 29414.54 | 4.81E−03 | 0571 |
| | (Shugoshin 1AB protein) | | | | |
| 684 | Q5H9B9 (Q5H9B9) BMP2 inducible kinase-like (Fragment) | Q5H9B9 | 45747.61 | 9.00E−03 | 0535 |
| 685 | Q5JQM8 (Q5JQM8) Complement component 4A | Q5JQM8 | 192663.6 | 2.32E−07 | 0556 |
| 686 | Q5JR12 (Q5JR12) Novel protein | Q5JR12 | 54799.88 | 7.16E−03 | 0556 |
| 687 | Q5JR20 (Q5JR20) Deleted in malignant brain tumors 1 | Q5JR20 | 260464.6 | 1.08E−06 | 0552 |
| 688 | Q5JR58 (Q5JR58) Glyceraldehyde 3-phosphate dehydrogenase | Q5JR58 | 36686.48 | 4.72E−03 | 0560 |
| | (GAPDH) | | | | |
| 689 | Q5JRA5 (Q5JRA5) C219-reactive peptide (FLJ39207) | Q5JRA5 | 56548.96 | 9.28E−03 | 0571 |
| 690 | KCNT1_HUMAN (Q5JUK3) Potassium channel subfamily T member 1 | Q5JUK3 | 138254.3 | 4.00E−03 | 0571 |
| | (KCa4.1) | | | | |
| 691 | Q5JUN8 (Q5JUN8) Protein kinase C, theta (Fragment) | Q5JUN8 | 55350.32 | 3.63E−03 | 0571 |
| 692 | Q5JVG2 (Q5JVG2) Zinc finger protein 484 | Q5JVG2 | 98157.78 | 7.77E−03 | 0552 |
| 693 | Q5JYA3 (Q5JYA3) Methylenetetrahydrofolate dehydrogenase | Q5JYA3 | 12417.52 | 3.22E−03 | 0560 |
| | (NADP+ dependent) 1-like (Fragment) | | | | |
| 694 | Q5JZ84 (Q5JZ84) OTTHUMP00000028514 (Fragment) | Q5JZ84 | 83801.98 | 1.58E−04 | 0549 |
| 695 | Q5NV92 (Q5NV92) V5-6 protein (Fragment) | Q5NV92 | 10567.04 | 2.66E−05 | 0535 |
| 696 | Q5QGN2 (Q5QGN2) HSPC069 isoform b | Q5QGN2 | 135851 | 2.88E−03 | 0541 |
| 697 | Q5RI13 (Q5RI13) Novel protein (FLJ36760) | Q5RI13 | 68256 | 5.57E−03 | 0556 |
| 698 | Q5SW71 (Q5SW71) Novel protein | Q5SW71 | 15510.08 | 1.16E−03 | 0556 |
| 699 | Q5T3P5 (Q5T3P5) Lectin, galactoside-binding, soluble, 8 (Galectin 8) | Q5T3P5 | 40371.93 | 7.60E−03 | 0567 |
| 700 | Q5T3U5 (Q5T3U5) ATP-binding cassette, sub-family C (CFTR/MRP), | Q5T3U5 | 161526.7 | 7.82E−03 | 0541 |
| | member 10 | | | | |
| 701 | Q5T583 (Q5T583) Filaggrin | Q5T583 | 434921.4 | 3.44E−03 | 0574 |
| 702 | Q5T7V5 (Q5T7V5) OTTHUMP00000021437 (Fragment) | Q5T7V5 | 32396.05 | 3.45E−05 | 0552 |
| 703 | Q5TAT4 (Q5TAT4) Collagen, type XIII, alpha 1 | Q5TAT4 | 66335.54 | 5.68E−03 | 0552 |
| 704 | Q5TGZ2 (Q5TGZ2) Neuroblastoma, suppression of tumorigenicity 1 | Q5TGZ2 | 19395.18 | 1.19E−08 | 0574 |
| 705 | Q5TI99 (Q5TI99) HBxAg transactivated protein 2 (XTP2) | Q5TI99 | 295818.6 | 2.64E−03 | 0541 |
| 706 | Q5UE57 (Q5UE57) Alcadein beta | Q5UE57 | 106966 | 2.12E−03 | 0541 |
| 707 | RIF1_HUMAN (Q5UIP0) Telomere-associated protein RIF1 (Rap1- | Q5UIP0 | 274293 | 3.33E−03 | 0571 |
| | interacting factor 1 homolog) | | | | |
| 708 | Q5VSR9 (Q5VSR9) Novel protein similar to Gorilla gorilla SPANX N | Q5VSR9 | 8257.914 | 2.10E−03 | 0574 |
| | member 1 (SPANX-N1) | | | | |
| 709 | Q5VUL6 (Q5VUL6) Filamin A interacting protein 1 | Q5VUL6 | 127127.2 | 5.43E−03 | 0567 |
| 710 | Q5VV35 (Q5VV35) Sarcomeric protein myopalladin, 145 kDa (MYOP) | Q5VV35 | 145166.5 | 5.43E−03 | 0571 |
| 711 | Q5VV84 (Q5VV84) Protein tyrosine phosphatase, receptor type, f | Q5VV84 | 134709.6 | 7.99E−03 | 0578 |
| | polypeptide (PTPRF), interacting pro | | | | |
| 712 | Q5VXU2 (Q5VXU2) Propionyl Coenzyme A carboxylase, alpha | Q5VXU2 | 80235.37 | 4.21E−03 | 0556 |
| | polypeptide | | | | |
| 713 | CI084_HUMAN (Q5VXU9) Hypothetical protein C9orf84 | Q5VXU9 | 165096.9 | 1.90E−04 | 0541 |
| 714 | Q5VYV7 (Q5VYV7) OTTHUMP00000030279 | Q5VYV7 | 45523.54 | 6.48E−03 | 0578 |
| 715 | Q5VZ60 (Q5VZ60) SMC5 structural maintenance of chromosomes 5- | Q5VZ60 | 128725.4 | 9.63E−03 | 0535 |
| | like 1 (Yeast) | | | | |
| 716 | Q5VZP5 (Q5VZP5) Novel protein | Q5VZP5 | 130096 | 1.22E−03 | 0574 |
| 717 | Q5VZV4 (Q5VZV4) OTTHUMP00000021537 | Q5VZV4 | 19621.98 | 1.41E−03 | 0574 |
| 718 | Q5W025 (Q5W025) Breast cancer antigen NY-BR-1 | Q5W025 | 152577.7 | 3.02E−03 | 0567 |
| 719 | Q64FX8 (Q64FX8) AKNA transcript D | Q64FX8 | 155043.7 | 6.17E−03 | 0560 |
| 720 | Q66K66 (Q66K66) Similar to RIKEN cDNA A230078I05 gene | Q66K66 | 39449.22 | 9.20E−04 | 0556 |
| 721 | Q66K74 (Q66K74) BPY2 interacting protein 1 | Q66K74 | 112158.2 | 1.08E−03 | 0549 |
| 722 | Q68BL7 (Q68BL7) Photomedin-1 | Q68BL7 | 73008.82 | 3.36E−03 | 0578 |
| 723 | Q68CR1 (Q68CR1) Hypothetical protein DKFZp781J1697 | Q68CR1 | 125119.3 | 6.96E−03 | 0574 |
| 724 | Q68CZ6 (Q68CZ6) Hypothetical protein DKFZp686I1868 | Q68CZ6 | 69606.88 | 5.97E−03 | 0560 |
| 725 | Q68DN6 (Q68DN6) Hypothetical protein DKFZp781D1923 | Q68DN6 | 196563.1 | 2.98E−03 | 0552 |
| 726 | Q68DS5 (Q68DS5) Hypothetical protein DKFZp686L17246 | Q68DS5 | 75999.35 | 9.04E−03 | 0560 |
| 727 | Q68DX7 (Q68DX7) Hypothetical protein DKFZp686E0722 (Fragment) | Q68DX7 | 238959.4 | 7.57E−03 | 0552 |
| 728 | Q695L1 (Q695L1) Striated muscle preferentially expressed protein | Q695L1 | 335326.6 | 3.70E−03 | 0549 |
| | (Fragment) | | | | |
| 729 | Q6AI27 (Q6AI27) Hypothetical protein DKFZp686D19113 | Q6AI27 | 18340.52 | 9.00E−03 | 0552 |
| 730 | Q6GMX5 (Q6GMX5) IGHM protein | Q6GMX5 | 65263.74 | 4.28E−11 | 0571 |
| 731 | Q6IA35 (Q6IA35) TOE1 protein | Q6IA35 | 56542.05 | 1.06E−03 | 0560 |
| 732 | Q6IEW7 (Q6IEW7) Olfactory receptor OR11-318 | Q6IEW7 | 36363.73 | 5.13E−03 | 0560 |
| 733 | Q6IFC7 (Q6IFC7) Olfactory receptor OR11-178 | Q6IFC7 | 38321 | 7.40E−03 | 0571 |
| 734 | Q6IPW0 (Q6IPW0) FLJ10404 protein (Fragment) | Q6IPW0 | 53802.47 | 1.90E−03 | 0541 |
| 735 | Q6IQ38 (Q6IQ38) USP36 protein | Q6IQ38 | 122732.4 | 1.53E−03 | 0541 |
| 736 | Q6MZQ6 (Q6MZQ6) Hypothetical protein DKFZp686G11190 | Q6MZQ6 | 52010 | 2.21E−05 | 0535 |
| 737 | Q6N041 (Q6N041) Hypothetical protein DKFZp686O16217 | Q6N041 | 54090.52 | 6.94E−05 | 0571 |
| | (Fragment) | | | | |
| 738 | Q6NW26 (Q6NW26) KCTD18 protein | Q6NW26 | 46707.95 | 4.66E−03 | 0556 |
| 739 | Q6NW33 (Q6NW33) KRT19 protein (Fragment) | Q6NW33 | 46179.44 | 1.14E−04 | 0552 |
| 740 | Q6NXG6 (Q6NXG6) NUP210 protein | Q6NXG6 | 105744 | 8.93E−03 | 0567 |
| 741 | Q6P5Q7 (Q6P5Q7) LOC493860 protein | Q6P5Q7 | 45455.3 | 9.77E−03 | 0541 |
| 742 | Q6P6A7 (Q6P6A7) LOC152217 protein (Hypothetical protein | Q6P6A7 | 10884 | 7.46E−03 | 0541 |
| | DKFZp762I1415) | | | | |
| 743 | Q6P714 (Q6P714) Hypothetical protein | Q6P714 | 7455.124 | 3.48E−03 | 0541 |
| 744 | Q6PJ04 (Q6PJ04) DKFZP434H0115 protein | Q6PJ04 | 33022.06 | 5.07E−04 | 0541 |
| 745 | Q6PJQ5 (Q6PJQ5) Forkhead box R2 | Q6PJQ5 | 35901.31 | 5.28E−03 | 0560 |
| 746 | Q6QNJ9 (Q6QNJ9) Steroid acute regulatory protein (Fragment) | Q6QNJ9 | 3219.632 | 4.26E−03 | 0574 |
| 747 | Q6TXQ4 (Q6TXQ4) H3L-like histone (Fragment) | Q6TXQ4 | 12096.65 | 1.69E−07 | 0560 |
| 748 | ANR12_HUMAN (Q6UB98) Ankyrin repeat domain protein 12 (Ankyrin | Q6UB98 | 235518.9 | 6.66E−03 | 0567 |
| | repeat-containing cofactor 2) (GAC-1 | | | | |
| 749 | Q6UTX4 (Q6UTX4) Hypothetical protein | Q6UTX4 | 32526.76 | 6.91E−04 | 0549 |
| 750 | ARSK_HUMAN (Q6UWY0) Arylsulfatase K precursor (EC 3.1.6.—) | Q6UWY0 | 61411.18 | 8.34E−03 | 0556 |
| | (ASK) (Telethon sulfatase) | | | | |
| 751 | Q6UXE9 (Q6UXE9) EQYK340 | Q6UXE9 | 94970.7 | 2.31E−03 | 0571 |
| 752 | Q6V0I7 (Q6V0I7) Fat-like cadherin FATJ protein | Q6V0I7 | 350970.7 | 3.36E−03 | 0574 |
| 753 | Q6ZMY0 (Q6ZMY0) Hypothetical protein FLJ16598 | Q6ZMY0 | 150875.1 | 2.71E−03 | 0549 |
| 754 | Q6ZMY7 (Q6ZMY7) Hypothetical protein FLJ16577 | Q6ZMY7 | 30088.58 | 7.13E−03 | 0571 |
| 755 | Q6ZN00 (Q6ZN00) Hypothetical protein FLJ16553 | Q6ZN00 | 21580.88 | 5.42E−03 | 0541 |
| 756 | Q6ZN07 (Q6ZN07) Hypothetical protein FLJ16540 | Q6ZN07 | 56924.62 | 2.37E−03 | 0549 |
| 757 | FGD5_HUMAN (Q6ZNL6) FYVE, RhoGEF and PH domain containing | Q6ZNL6 | 159806.6 | 5.65E−03 | 0567 |
| | protein 5 (Zinc finger FYVE domain contai | | | | |
| 758 | Q6ZR22 (Q6ZR22) Hypothetical protein FLJ46715 | Q6ZR22 | 148787.5 | 6.45E−03 | 0567 |
| 759 | Q6ZR59 (Q6ZR59) Hypothetical protein FLJ46623 | Q6ZR59 | 14124.17 | 7.53E−03 | 0549 |
| 760 | Q6ZRB9 (Q6ZRB9) Hypothetical protein FLJ46489 | Q6ZRB9 | 27860.95 | 7.73E−03 | 0535 |
| 761 | Q6ZRF6 (Q6ZRF6) Hypothetical protein FLJ46386 | Q6ZRF6 | 21524.64 | 1.11E−03 | 0567 |
| 762 | Q6ZRG5 (Q6ZRG5) Hypothetical protein FLJ46369 | Q6ZRG5 | 24107.1 | 7.11E−04 | 0541 |
| 763 | Q6ZRJ1 (Q6ZRJ1) Hypothetical protein FLJ46323 | Q6ZRJ1 | 111189.1 | 4.80E−04 | 0556 |
| 764 | Q6ZRR7 (Q6ZRR7) Hypothetical protein FLJ46156 | Q6ZRR7 | 128052.8 | 5.12E−03 | 0560 |
| 765 | Q6ZRV2 (Q6ZRV2) Hypothetical protein FLJ46072 | Q6ZRV2 | 94258.88 | 5.38E−03 | 0574 |
| 766 | Q6ZRZ7 (Q6ZRZ7) Hypothetical protein FLJ45950 | Q6ZRZ7 | 14798.13 | 7.18E−03 | 0552 |
| 767 | Q6ZRZ8 (Q6ZRZ8) CDNA FLJ45949 fis, clone PLACE7007973 | Q6ZRZ8 | 54349.71 | 7.27E−03 | 0571 |
| 768 | Q6ZST2 (Q6ZST2) Hypothetical protein FLJ45231 | Q6ZST2 | 14400.26 | 2.83E−03 | 0556 |
| 769 | Q6ZT81 (Q6ZT81) Hypothetical protein FLJ44884 | Q6ZT81 | 25240.86 | 5.58E−04 | 0541 |
| 770 | Q6ZTK3 (Q6ZTK3) Hypothetical protein FLJ44574 | Q6ZTK3 | 110872 | 5.85E−03 | 0578 |
| 771 | Q6ZUD4 (Q6ZUD4) Hypothetical protein FLJ43798 | Q6ZUD4 | 15462.47 | 6.23E−03 | 0578 |
| 772 | Q6ZVG1 (Q6ZVG1) Hypothetical protein FLJ42621 | Q6ZVG1 | 78363.38 | 4.81E−03 | 0541 |
| 773 | Q6ZVT0 (Q6ZVT0) Hypothetical protein FLJ42131 | Q6ZVT0 | 74946.23 | 2.71E−03 | 0541 |
| 774 | TBA3_HUMAN (Q71U36) Tubulin alpha-3 chain (Alpha-tubulin 3) | Q71U36 | 50103.65 | 2.47E−03 | 0552 |
| | (Tubulin B-alpha-1) | | | | |
| 775 | MARK2_HUMAN (Q7KZI7) Serine/threonine-protein kinase MARK2 | Q7KZI7 | 87855.99 | 7.95E−03 | 0578 |
| | (EC 2.7.1.37) (MAP/microtubule affinity- | | | | |
| 776 | CHST3_HUMAN (Q7LGC8) Carbohydrate sulfotransferase 3 (EC | Q7LGC8 | 54638.37 | 3.38E−03 | 0560 |
| | 2.8.2.17) (Chondroitin 6-sulfotransferase) | | | | |
| 777 | Q7RTR4 (Q7RTR4) NOD16 | Q7RTR4 | 119351.7 | 5.77E−03 | 0560 |
| 778 | Q7RTS7 (Q7RTS7) Keratin 5c | Q7RTS7 | 59333.45 | 7.49E−03 | 0574 |
| 779 | Q7Z353 (Q7Z353) Hypothetical protein DKFZp686K21156 | Q7Z353 | 77157.85 | 1.10E−03 | 0549 |
| 780 | Q7Z374 (Q7Z374) Hypothetical protein DKFZp686C02218 (Fragment) | Q7Z374 | 53741.82 | 2.73E−12 | 0571 |
| 781 | Q7Z379 (Q7Z379) Hypothetical protein DKFZp686K04218 (Fragment) | Q7Z379 | 51587.73 | 8.88E−04 | 0560 |
| 782 | Q7Z3E7 (Q7Z3E7) Hypothetical protein DKFZp686K091 | Q7Z3E7 | 245494.7 | 2.29E−03 | 0552 |
| 783 | Q7Z3I3 (Q7Z3I3) Hypothetical protein DKFZp686F07114 | Q7Z3I3 | 123251.3 | 2.63E−03 | 0567 |
| 784 | Q7Z3Q5 (Q7Z3Q5) Hypothetical protein DKFZp313G216 | Q7Z3Q5 | 57159.47 | 2.63E−03 | 0574 |
| 785 | Q7Z431 (Q7Z431) Putative NFkB activating protein | Q7Z431 | 39758.47 | 3.81E−03 | 0560 |
| 786 | GLT15_HUMAN (Q7Z4T8) Probable N- | Q7Z4T8 | 51447.66 | 7.55E−03 | 0574 |
| | acetylgalactosaminyltransferase 15 (EC 2.4.1.—) (Protein-UDP acetyl | | | | |
| 787 | CN029_HUMAN (Q7Z5M8) Protein C14orf29 | Q7Z5M8 | 40749.98 | 6.91E−03 | 0567 |
| 788 | Q7Z5R6 (Q7Z5R6) Amyloid beta (A4) protein-binding, family B, | Q7Z5R6 | 73137.02 | 8.83E−03 | 0560 |
| | member 1 interacting protein | | | | |
| 789 | Q7Z6B9 (Q7Z6B9) ZNF618 protein (Fragment) | Q7Z6B9 | 96694.38 | 2.86E−03 | 0552 |
| 790 | GRASP_HUMAN (Q7Z6J2) General receptor for phosphoinositides 1- | Q7Z6J2 | 42597.13 | 9.65E−04 | 0574 |
| | associated scaffold protein (GRP1-ass | | | | |
| 791 | Q86T80 (Q86T80) Hypothetical protein DKFZp451M0318 | Q86T80 | 40591.57 | 1.39E−03 | 0549 |
| 792 | TTC6_HUMAN (Q86TZ1) Tetratricopeptide repeat protein 6 (TPR | Q86TZ1 | 59340.45 | 4.63E−03 | 0552 |
| | repeat protein 6) | | | | |
| 793 | Q86UP3 (Q86UP3) Zinc finger homeodomain 4 protein | Q86UP3 | 393481.6 | 5.15E−03 | 0567 |
| 794 | Q86UW7 (Q86UW7) Ca2+-dependent activator protein for secretion 2 | Q86UW7 | 147548.8 | 6.37E−05 | 0560 |
| 795 | IQGA3_HUMAN (Q86VI3) Ras GTPase-activating-like protein | Q86VI3 | 184430.7 | 4.73E−03 | 0552 |
| | IQGAP3 | | | | |
| 796 | Q86WM1 (Q86WM1) Zinc finger protein ZNF284 (Fragment) | Q86WM1 | 23565.48 | 4.66E−03 | 0578 |
| 797 | Q86WN2 (Q86WN2) Interferon epsilon-1 (Interferon-epsilon) | Q86WN2 | 24398.87 | 2.14E−03 | 0552 |
| 798 | ZN227_HUMAN (Q86WZ6) Zinc finger protein 227 | Q86WZ6 | 91973.55 | 3.55E−03 | 0578 |
| 799 | Q86Y06 (Q86Y06) Sister-of-mammalian grainyhead isoform 1 | Q86Y06 | 68275.58 | 4.30E−03 | 0571 |
| 800 | GPR97_HUMAN (Q86Y34) Probable G-protein coupled receptor 97 | Q86Y34 | 60821.61 | 3.61E−03 | 0552 |
| | precursor (G-protein coupled receptor P | | | | |
| 801 | XYLT1_HUMAN (Q86Y38) Xylosyltransferase 1 (EC 2.4.2.26) | Q86Y38 | 107501.6 | 9.95E−03 | 0541 |
| | (Xylosyltransferase I) (XylT-I) (XT-I) (Pep | | | | |
| 802 | Q86YK7 (Q86YK7) Heat shock regulated-1 | Q86YK7 | 222995.4 | 5.99E−03 | 0574 |
| 803 | Q8IU65 (Q8IU65) Axonemal dynein heavy chain 8 isoform 2 | Q8IU65 | 23934.03 | 9.23E−03 | 0556 |
| | (Axonemal dynein heavy chain 8 isoform 3) ( | | | | |
| 804 | Q8IXR4 (Q8IXR4) LOC388906 protein (Fragment) | Q8IXR4 | 23682.2 | 6.65E−03 | 0567 |
| 805 | Q8IY89 (Q8IY89) FLJ21062 protein | Q8IY89 | 25602.36 | 3.70E−03 | 0552 |
| 806 | ZN440_HUMAN (Q8IYI8) Zinc finger protein 440 | Q8IYI8 | 69060.77 | 3.73E−03 | 0549 |
| 807 | Q8IZA4 (Q8IZA4) ELYS transcription factor-like protein TMBS62 | Q8IZA4 | 256022.6 | 1.35E−03 | 0567 |
| 808 | CN039_HUMAN (Q8N1H7) Hypothetical protein C14orf39 | Q8N1H7 | 68199.46 | 9.14E−03 | 0552 |
| 809 | Q8N392 (Q8N392) Hypothetical protein DKFZp762I167 | Q8N392 | 74900.02 | 9.10E−03 | 0560 |
| 810 | Q8N393 (Q8N393) Hypothetical protein DKFZp762I137 | Q8N393 | 89778.09 | 3.80E−03 | 0567 |
| 811 | MILK1_HUMAN (Q8N3F8) Molecule interacting with Rab13 | Q8N3F8 | 93383.18 | 3.21E−03 | 0535 |
| | (MIRab13) (MICAL-like protein 1) | | | | |
| 812 | Q8N3I3 (Q8N3I3) Hypothetical protein DKFZp761P18121 | Q8N3I3 | 81550.7 | 1.87E−03 | 0552 |
| 813 | Q8N4J6 (Q8N4J6) KIAA1205 protein (Fragment) | Q8N4J6 | 65079.45 | 3.22E−03 | 0541 |
| 814 | Q8N4L2 (Q8N4L2) Hypothetical protein TMEM55A | Q8N4L2 | 28062.26 | 4.65E−03 | 0578 |
| 815 | Q8N549 (Q8N549) Hypothetical protein C8orf36 | Q8N549 | 27915 | 5.67E−03 | 0556 |
| 816 | Q8N5C6 (Q8N5C6) FLJ10379 protein | Q8N5C6 | 69669.68 | 3.13E−04 | 0552 |
| 817 | LZTR1_HUMAN (Q8N653) Leucine-zipper-like transcriptional | Q8N653 | 94658.3 | 2.08E−03 | 0556 |
| | regulator 1 (LZTR-1) | | | | |
| 818 | Q8N6E7 (Q8N6E7) KIAA1430 protein | Q8N6E7 | 50506.21 | 3.91E−04 | 0556 |
| 819 | Q8N780 (Q8N780) Hypothetical protein FLJ25943 | Q8N780 | 33347.2 | 7.92E−03 | 0535 |
| 820 | Q8N7A6 (Q8N7A6) Hypothetical protein FLJ25862 | Q8N7A6 | 21106.6 | 1.15E−03 | 0549 |
| 821 | Q8N7S0 (Q8N7S0) Hypothetical protein FLJ40424 | Q8N7S0 | 13945.2 | 8.51E−03 | 0541 |
| 822 | Q8N819 (Q8N819) Hypothetical protein FLJ40125 | Q8N819 | 37141.55 | 4.13E−03 | 0552 |
| 823 | Q8N853 (Q8N853) Hypothetical protein FLJ40006 | Q8N853 | 18183.24 | 1.02E−03 | 0574 |
| 824 | Q8N8V3 (Q8N8V3) Hypothetical protein FLJ38820 | Q8N8V3 | 20250.83 | 5.60E−03 | 0567 |
| 825 | Q8N905 (Q8N905) Hypothetical protein FLJ38620 | Q8N905 | 42098.63 | 5.67E−04 | 0549 |
| 826 | Q8N951 (Q8N951) Hypothetical protein FLJ38360 | Q8N951 | 48744.87 | 3.01E−03 | 0567 |
| 827 | Q8NA95 (Q8NA95) Hypothetical protein FLJ35725 | Q8NA95 | 41300.77 | 2.84E−03 | 0560 |
| 828 | SMYD1_HUMAN (Q8NB12) SET and MYND domain containing | Q8NB12 | 56579.95 | 6.20E−03 | 0549 |
| | protein 1 | | | | |
| 829 | UBP38_HUMAN (Q8NB14) Ubiquitin carboxyl-terminal hydrolase 38 | Q8NB14 | 116472.1 | 1.24E−03 | 0549 |
| | (EC 3.1.2.15) (Ubiquitin thiolesteras | | | | |
| 830 | S4A11_HUMAN (Q8NBS3) Sodium bicarbonate transporter-like | Q8NBS3 | 99517.14 | 6.08E−03 | 0560 |
| | protein 11 (Bicarbonate transporter-relate | | | | |
| 831 | SCPDH_HUMAN (Q8NBX0) Probable saccharopine dehydrogenase | Q8NBX0 | 47121.48 | 6.82E−03 | 0541 |
| | (EC 1.5.1.9) | | | | |
| 832 | Q8NC98 (Q8NC98) Hypothetical protein FLJ90397 | Q8NC98 | 36994.11 | 4.44E−03 | 0549 |
| 833 | Q8ND50 (Q8ND50) Hypothetical protein DKFZp547N203 (Fragment) | Q8ND50 | 105208.9 | 1.39E−06 | 0541 |
| 834 | Q8NDA2 (Q8NDA2) Hypothetical protein DKFZp434P0216 | Q8NDA2 | 143799.3 | 3.16E−03 | 0560 |
| | (Fragment) | | | | |
| 835 | ABCF1_HUMAN (Q8NE71) ATP-binding cassette sub-family F | Q8NE71 | 95866.58 | 1.91E−03 | 0535 |
| | member 1 (ATP-binding cassette 50) (TNF-alph | | | | |
| 836 | NFAM1_HUMAN (Q8NET5) NFAT activation molecule 1 precursor | Q8NET5 | 29667.17 | 5.08E−03 | 0541 |
| | (Calcineurin/NFAT-activating ITAM-contain | | | | |
| 837 | Q8NF06 (Q8NF06) FLJ00398 protein (Fragment) | Q8NF06 | 72000.41 | 8.89E−03 | 0552 |
| 838 | SYNE1_HUMAN (Q8NF91) Nesprin-1 (Nuclear envelope spectrin | Q8NF91 | 1010433 | 4.94E−03 | 0552 |
| | repeat protein 1) (Synaptic nuclear envel | | | | |
| 839 | VMDL1_HUMAN (Q8NFU1) Bestrophin-2 (Vitelliform macular | Q8NFU1 | 57101.98 | 3.93E−03 | 0535 |
| | dystrophy 2-like protein 1) | | | | |
| 840 | Q8NFY8 (Q8NFY8) Neuroblastoma-amplified protein | Q8NFY8 | 268295.4 | 3.08E−03 | 0560 |
| 841 | NLGN2_HUMAN (Q8NFZ4) Neuroligin-2 precursor | Q8NFZ4 | 90763.22 | 6.71E−04 | 0574 |
| 842 | OR6M1_HUMAN (Q8NGM8) Olfactory receptor 6M1 (Olfactory | Q8NGM8 | 35304.85 | 5.61E−03 | 0541 |
| | receptor OR11-271) | | | | |
| 843 | GP150_HUMAN (Q8NGU9) Probable G-protein coupled receptor 150 | Q8NGU9 | 46323.67 | 5.89E−04 | 0578 |
| 844 | WDR36_HUMAN (Q8NI36) WD-repeat protein 36 (T-cell activation | Q8NI36 | 105255.2 | 4.25E−03 | 0552 |
| | WD repeat protein) (TA-WDRP) | | | | |
| 845 | Q8TAL6 (Q8TAL6) Hypothetical protein LOC387758 (Hypothetical | Q8TAL6 | 24257.93 | 5.39E−03 | 0541 |
| | protein HEMBA1007053) | | | | |
| 846 | TIP_HUMAN (Q8TB96) T-cell immunomodulatory protein precursor | Q8TB96 | 68064.6 | 5.32E−05 | 0552 |
| | (TIP protein) | | | | |
| 847 | Q8TBY0 (Q8TBY0) Hypothetical protein MGC27016 | Q8TBY0 | 59984.24 | 7.31E−03 | 0578 |
| 848 | Q8TBY6 (Q8TBY6) MGC26597 protein | Q8TBY6 | 56738.82 | 6.82E−03 | 0541 |
| 849 | Q8TCM1 (Q8TCM1) Hypothetical protein DKFZp547K059 | Q8TCM1 | 85774.15 | 9.30E−03 | 0552 |
| 850 | Q8TD57 (Q8TD57) Axonemal heavy chain dynein type 3 | Q8TD57 | 470467.1 | 8.03E−03 | 0556 |
| 851 | Q8TDP0 (Q8TDP0) Apical protein 2 | Q8TDP0 | 90246 | 8.44E−03 | 0574 |
| 852 | LMX1A_HUMAN (Q8TE12) LIM homeobox transcription factor 1 alpha | Q8TE12 | 42719.79 | 1.26E−03 | 0549 |
| | (LIM/homeobox protein LMX1A) (LIM-ho | | | | |
| 853 | ATS16_HUMAN (Q8TE57) ADAMTS-16 precursor (EC 3.4.24.—) (A | Q8TE57 | 136098.1 | 7.41E−03 | 0549 |
| | disintegrin and metalloproteinase with th | | | | |
| 854 | ATS15_HUMAN (Q8TE58) ADAMTS-15 precursor (EC 3.4.24.—) (A | Q8TE58 | 103220.4 | 3.54E−03 | 0574 |
| | disintegrin and metalloproteinase with th | | | | |
| 855 | Q8TF44 (Q8TF44) KIAA1957 protein (Fragment) | Q8TF44 | 51192.71 | 9.00E−03 | 0574 |
| 856 | Q8WTR8 (Q8WTR8) Hypothetical protein LOC126147 | Q8WTR8 | 53139.27 | 7.76E−04 | 0535 |
| 857 | NU133_HUMAN (Q8WUM0) Nuclear pore complex protein Nup133 | Q8WUM0 | 128931.9 | 6.66E−03 | 0549 |
| | (Nucleoporin Nup133) (133 kDa nucleoporin) | | | | |
| 858 | HNRLL_HUMAN (Q8WVV9) Heterogeneous nuclear | Q8WVV9 | 60044.95 | 1.76E−03 | 0556 |
| | ribonucleoprotein L-like (Stromal RNA-regulating factor) | | | | |
| 859 | PCNP_HUMAN (Q8WW12) PEST-containing nuclear protein (PCNP) | Q8WW12 | 18913.4 | 6.54E−04 | 0541 |
| 860 | FOG2_HUMAN (Q8WW38) Zinc finger protein ZFPM2 (Zinc finger | Q8WW38 | 128061.4 | 1.37E−04 | 0571 |
| | protein multitype 2) (Friend of GATA pro | | | | |
| 861 | Q8WWF6 (Q8WWF6) Hypothetical protein HCG3 (Hypothetical | Q8WWF6 | 16548.14 | 4.34E−03 | 0552 |
| | protein tmp_locus_21) | | | | |
| 862 | ATX2L_HUMAN (Q8WWM7) Ataxin-2-like protein (Ataxin-2 domain | Q8WWM7 | 113303.8 | 4.89E−03 | 0560 |
| | protein) (Ataxin-2-related protein) | | | | |
| 863 | ASB12_HUMAN (Q8WXK4) Ankyrin repeat and SOCS box protein 12 | Q8WXK4 | 33921.47 | 2.64E−04 | 0541 |
| | (ASB-12) | | | | |
| 864 | Q8WXQ5 (Q8WXQ5) Serine/threonine protein kinase kkialre-like 1 | Q8WXQ5 | 108892.4 | 9.25E−03 | 0578 |
| 865 | Q8WXX0 (Q8WXX0) Ciliary dynein heavy chain 7 | Q8WXX0 | 460845.6 | 6.97E−03 | 0541 |
| 866 | Q8WY12 (Q8WY12) B lymphocyte activation-related protein | Q8WY12 | 40553.01 | 8.90E−03 | 0549 |
| | (Transcription elongation factor A (SII)-li | | | | |
| 867 | Q8WZ93 (Q8WZ93) NAG11 | Q8WZ93 | 9833.928 | 4.21E−03 | 0560 |
| 868 | GBF1_HUMAN (Q92538) Golgi-specific brefeldin A-resistance | Q92538 | 206313.1 | 5.67E−03 | 0578 |
| | guanine nucleotide exchange factor 1 (BFA | | | | |
| 869 | TIC2_HUMAN (Q92563) Testican-2 precursor (SPARC/osteonectin, | Q92563 | 46748.9 | 1.09E−04 | 0541 |
| | CWCV, and Kazal-like domains proteogly | | | | |
| 870 | RT31_HUMAN (Q92665) 28S ribosomal protein S31, mitochondrial | Q92665 | 45272.55 | 5.95E−03 | 0541 |
| | precursor (S31mt) (MRP-S31) (Imogen 38 | | | | |
| 871 | PVR2_HUMAN (Q92692) Poliovirus receptor related protein 2 | Q92692 | 57706.01 | 8.45E−03 | 0571 |
| | precursor (Herpes virus entry mediator B) | | | | |
| 872 | CBP_HUMAN (Q92793) CREB-binding protein (EC 2.3.1.48) | Q92793 | 265166 | 2.79E−03 | 0556 |
| 873 | PTPR2_HUMAN (Q92932) Receptor-type tyrosine-protein | Q92932 | 111212.2 | 9.78E−04 | 0556 |
| | phosphatase N2 precursor (EC 3.1.3.48) (R-PTP-N | | | | |
| 874 | Q92954 (Q92954) Megakaryocyte stimulating factor | Q92954 | 150997.2 | 3.09E−03 | 0556 |
| 875 | USP9X_HUMAN (Q93008) Probable ubiquitin carboxyl-terminal | Q93008 | 289354.6 | 1.75E−03 | 0552 |
| | hydrolase FAF-X (EC 3.1.2.15) (Ubiquitin | | | | |
| 876 | BHMT_HUMAN (Q93088) Betaine-homocysteine S- | Q93088 | 44941.84 | 5.57E−03 | 0552 |
| | methyltransferase (EC 2.1.1.5) | | | | |
| 877 | Q969F2 (Q969F2) Dvl-binding protein NKD2 (Naked cuticle-2) | Q969F2 | 50024.88 | 5.30E−03 | 0578 |
| 878 | KIFC2_HUMAN (Q96AC6) Kinesin-like protein KIFC2 | Q96AC6 | 90091.98 | 8.85E−03 | 0541 |
| 879 | TTC17_HUMAN (Q96AE7) Tetratricopeptide repeat protein 17 (TPR | Q96AE7 | 129476.8 | 3.84E−03 | 0574 |
| | repeat protein 17) | | | | |
| 880 | Q96BE9 (Q96BE9) CDK4 protein | Q96BE9 | 11993.24 | 5.74E−03 | 0552 |
| 881 | SYTL4_HUMAN (Q96C24) Synaptotagmin-like protein 4 (Exophilin-2) | Q96C24 | 75961.86 | 6.03E−03 | 0552 |
| | (Granuphilin) | | | | |
| 882 | Q96CM8 (Q96CM8) Hypothetical protein FLJ20920 | Q96CM8 | 68122.89 | 3.83E−03 | 0541 |
| 883 | Q96DB1 (Q96DB1) TALDO1 protein | Q96DB1 | 35306.46 | 9.85E−03 | 0578 |
| 884 | Q96ES7 (Q96ES7) Hypothetical protein LOC112869 | Q96ES7 | 33217.24 | 9.31E−03 | 0552 |
| 885 | Q96FI6 (Q96FI6) Enhancer of zeste 2, isoform a | Q96FI6 | 85962.88 | 1.62E−03 | 0578 |
| 886 | CPNE2_HUMAN (Q96FN4) Copine-2 (Copine II) | Q96FN4 | 61150.76 | 3.05E−03 | 0541 |
| 887 | Q96G34 (Q96G34) DC12 protein | Q96G34 | 40535.21 | 3.58E−03 | 0552 |
| 888 | Q96JG3 (Q96JG3) KIAA1864 protein (Fragment) | Q96JG3 | 75937.59 | 8.79E−04 | 0556 |
| 889 | Q96JI8 (Q96JI8) KIAA1839 protein (Fragment) | Q96JI8 | 61235.62 | 2.21E−04 | 0571 |
| 890 | BT2A3_HUMAN (Q96KV6) Butyrophilin subfamily 2 member A3 | Q96KV6 | 63346.8 | 8.73E−04 | 0560 |
| | precursor | | | | |
| 891 | Q96LM3 (Q96LM3) Hypothetical protein FLJ25373 | Q96LM3 | 46043.74 | 8.00E−04 | 0552 |
| 892 | Q96LM6 (Q96LM6) Hypothetical protein FLJ25369 | Q96LM6 | 20601.17 | 2.00E−04 | 0549 |
| 893 | Q96MA6 (Q96MA6) Hypothetical protein FLJ32704 (Chromosome 9 | Q96MA6 | 54890.73 | 1.41E−03 | 0549 |
| | open reading frame 98) (OTTHUMP00000022 | | | | |
| 894 | YH01_HUMAN (Q96MG8) Hypothetical protein | Q96MG8 | 40648.61 | 3.78E−03 | 0567 |
| | FLJ32390/DKFZp686L23117 | | | | |
| 895 | PGRP2_HUMAN (Q96PD5) N-acetylmuramoyl-L-alanine amidase | Q96PD5 | 62178 | 1.31E−05 | 0556 |
| | precursor (EC 3.5.1.28) (Peptidoglycan reco | | | | |
| 896 | Q96Q34 (Q96Q34) ALS2CR13 protein (Fragment) | Q96Q34 | 21396.12 | 9.02E−03 | 0571 |
| 897 | Q96RY5 (Q96RY5) Hypothetical protein KIAA1426 | Q96RY5 | 169729.5 | 1.45E−04 | 0541 |
| 898 | Q96S02 (Q96S02) Hypothetical protein gs80 | Q96S02 | 25483.99 | 4.56E−03 | 0552 |
| 899 | Q96S07 (Q96S07) Hypothetical protein gs64 | Q96S07 | 40973.07 | 2.06E−03 | 0567 |
| 900 | TESK2_HUMAN (Q96S53) Dual specificity testis-specific protein | Q96S53 | 63599.28 | 3.27E−03 | 0574 |
| | kinase 2 (EC 2.7.1.37) (EC 2.7.1.112) | | | | |
| 901 | CCNL2_HUMAN (Q96S94) Cyclin-L2 (Paneth cell enhanced | Q96S94 | 58111.48 | 2.23E−03 | 0571 |
| | expression protein) | | | | |
| 902 | FIZ1_HUMAN (Q96SL8) Flt3-interacting zinc finger protein 1 | Q96SL8 | 51960.49 | 2.18E−03 | 0571 |
| 903 | SCN2A_HUMAN (Q99250) Sodium channel protein type II alpha | Q99250 | 227825.1 | 1.94E−03 | 0549 |
| | subunit (Voltage-gated sodium channel alp | | | | |
| 904 | Q99463 (Q99463) Y6 encoding protein | Q99463 | 33158.45 | 7.65E−03 | 0552 |
| 905 | SDF2_HUMAN (Q99470) Stromal cell-derived factor 2 precursor | Q99470 | 23154.53 | 2.53E−03 | 0549 |
| | (SDF-2) | | | | |
| 906 | NEUS_HUMAN (Q99574) Neuroserpin precursor (Serpin I1) | Q99574 | 46397.2 | 5.41E−04 | 0567 |
| | (Protease inhibitor 12) | | | | |
| 907 | ABCA3_HUMAN (Q99758) ATP-binding cassette sub-family A | Q99758 | 191263.9 | 6.35E−03 | 0552 |
| | member 3 (ATP-binding cassette transporter 3 | | | | |
| 908 | Q99777 (Q99777) Hypothetical protein (Fragment) | Q99777 | 14098.42 | 5.87E−03 | 0552 |
| 909 | TIG2_HUMAN (Q99969) Retinoic acid receptor responder protein 2 | Q99969 | 18605.75 | 1.02E−06 | 0567 |
| | precursor (Tazarotene-induced gene 2 | | | | |
| 910 | Q9BQ75 (Q9BQ75) Hypothetical protein MGC4308 | Q9BQ75 | 31791.96 | 8.74E−03 | 0541 |
| 911 | MBB1A_HUMAN (Q9BQG0) Myb-binding protein 1A | Q9BQG0 | 148761.2 | 4.19E−04 | 0541 |
| 912 | Q9BR99 (Q9BR99) Hypothetical protein LOC115648 | Q9BR99 | 11621.78 | 3.35E−03 | 0549 |
| 913 | Q9BX32 (Q9BX32) OTTHUMP00000030312 (Fragment) | Q9BX32 | 11352.71 | 4.49E−03 | 0567 |
| 914 | GPR91_HUMAN (Q9BXA5) Probable G-protein coupled receptor 91 | Q9BXA5 | 38258.2 | 6.04E−03 | 0574 |
| | (P2Y purinoceptor 1-like) | | | | |
| 915 | TSSK1_HUMAN (Q9BXA7) Testis-specific serine/threonine-protein | Q9BXA7 | 41591.27 | 4.61E−03 | 0541 |
| | kinase 1 (EC 2.7.1.37) (TSSK-1) (Test | | | | |
| 916 | RFIP5_HUMAN (Q9BXF6) Rab11 family-interacting protein 5 (Rab11- | Q9BXF6 | 70372.12 | 6.40E−03 | 0574 |
| | FIP5) (Rab11-interacting protein Rip | | | | |
| 917 | TX13A_HUMAN (Q9BXU3) Testis-expressed sequence 13A protein | Q9BXU3 | 45554.41 | 6.65E−03 | 0556 |
| 918 | CT043_HUMAN (Q9BY42) Protein C20orf43 | Q9BY42 | 33833.23 | 7.56E−03 | 0571 |
| 919 | Q9BZT1 (Q9BZT1) PNAS-101 | Q9BZT1 | 16324.19 | 2.85E−03 | 0535 |
| 920 | Q9C0D7 (Q9C0D7) KIAA1726 protein (Fragment) | Q9C0D7 | 70689.04 | 7.02E−03 | 0535 |
| 921 | AICDA_HUMAN (Q9GZX7) Activation-induced cytidine deaminase | Q9GZX7 | 23938.14 | 9.54E−03 | 0535 |
| | (EC 3.5.4.5) (Cytidine aminohydrolase) | | | | |
| 922 | ADA19_HUMAN (Q9H013) ADAM 19 precursor (EC 3.4.24.—) (A | Q9H013 | 104970.3 | 4.24E−03 | 0549 |
| | disintegrin and metalloproteinase domain 19 | | | | |
| 923 | Q9H0M5 (Q9H0M5) Hypothetical protein DKFZp434I1610 | Q9H0M5 | 86175.93 | 6.24E−03 | 0571 |
| 924 | CCDC8_HUMAN (Q9H0W5) Coiled-coil domain containing protein 8 | Q9H0W5 | 59367.06 | 7.08E−03 | 0578 |
| 925 | KCNH6_HUMAN (Q9H252) Potassium voltage-gated channel | Q9H252 | 109855.1 | 5.53E−03 | 0535 |
| | subfamily H member 6 (Voltage-gated potassium | | | | |
| 926 | ATS10_HUMAN (Q9H324) ADAMTS-10 precursor (EC 3.4.24.—) (A | Q9H324 | 117996.6 | 7.01E−03 | 0574 |
| | disintegrin and metalloproteinase with th | | | | |
| 927 | Q9H3M9 (Q9H3M9) Homo sapiens (Fragment) | Q9H3M9 | 39581.41 | 6.22E−03 | 0567 |
| 928 | HIPK3_HUMAN (Q9H422) Homeodomain-interacting protein kinase 3 | Q9H422 | 133658.7 | 3.99E−03 | 0549 |
| | (EC 2.7.1.37) (Homolog of protein kin | | | | |
| 929 | Q9H6S3 (Q9H6S3) Hypothetical protein FLJ21935 | Q9H6S3 | 83739.81 | 6.12E−04 | 0571 |
| 930 | Q9H878 (Q9H878) Hypothetical protein FLJ13893 | Q9H878 | 42146.18 | 1.26E−03 | 0541 |
| 931 | PANK3_HUMAN (Q9H999) Pantothenate kinase 3 (EC 2.7.1.33) | Q9H999 | 41067.9 | 3.46E−03 | 0535 |
| | (Pantothenic acid kinase 3) (hPanK3) | | | | |
| 932 | Q9H9E4 (Q9H9E4) Hypothetical protein FLJ12811 | Q9H9E4 | 59940.2 | 1.30E−03 | 0574 |
| 933 | Q9HA36 (Q9HA36) Hypothetical protein FLJ12303 | Q9HA36 | 63469.17 | 2.77E−03 | 0567 |
| 934 | POPD3_HUMAN (Q9HBV1) Popeye domain containing protein 3 | Q9HBV1 | 33848.33 | 2.18E−04 | 0571 |
| | (Popeye protein 3) | | | | |
| 935 | Q9HBY0 (Q9HBY0) Putative superoxide-generating NADPH oxidase | Q9HBY0 | 64893.13 | 3.83E−03 | 0560 |
| | Mox2 (NADPH oxidase 3) | | | | |
| 936 | ZSWM6_HUMAN (Q9HCJ5) Zinc finger SWIM domain containing | Q9HCJ5 | 84041.97 | 2.85E−03 | 0535 |
| | protein 6 (Fragment) | | | | |
| 937 | ZBT26_HUMAN (Q9HCK0) Zinc finger and BTB domain containing | Q9HCK0 | 49920.5 | 5.26E−03 | 0552 |
| | protein 26 (Zinc finger protein 481) (Zi | | | | |
| 938 | RFRP_HUMAN (Q9HCQ7) FMRFamide-related peptides precursor | Q9HCQ7 | 22312.28 | 1.20E−04 | 0567 |
| | [Contains: Neuropeptide NPSF (Neuropeptide | | | | |
| 939 | XAB2_HUMAN (Q9HCS7) XPA-binding protein 2 (HCNP protein) | Q9HCS7 | 99946.48 | 4.36E−03 | 0556 |
| 940 | Q9HD28 (Q9HD28) Retinitis pigmentosa GTPase regulator | Q9HD28 | 62722.3 | 5.89E−03 | 0541 |
| | (Fragment) | | | | |
| 941 | DMAP1_HUMAN (Q9NPF5) DNA methyltransferase 1-associated | Q9NPF5 | 52959.89 | 4.26E−03 | 0541 |
| | protein 1 (DNMT1-associated protein 1) (DNM | | | | |
| 942 | RTN4_HUMAN (Q9NQC3) Reticulon-4 (Neurite outgrowth inhibitor) | Q9NQC3 | 129851.2 | 6.83E−03 | 0578 |
| | (Nogo protein) (Foocen) (Neuroendocri | | | | |
| 943 | DDX4_HUMAN (Q9NQI0) DEAD-box protein 4 (VASA homolog) | Q9NQI0 | 79257.87 | 6.00E−03 | 0541 |
| 944 | Q9NQW6 (Q9NQW6) Actin binding protein anillin | Q9NQW6 | 124200.7 | 7.48E−04 | 0541 |
| 945 | ZN331_HUMAN (Q9NQX6) Zinc finger protein 331 (Zinc finger protein | Q9NQX6 | 53704.15 | 6.51E−03 | 0549 |
| | 463) (C2H2-like zinc finger prote | | | | |
| 946 | TLR9_HUMAN (Q9NR96) Toll-like receptor 9 precursor (CD289 | Q9NR96 | 115785.8 | 8.93E−03 | 0552 |
| | antigen) | | | | |
| 947 | SPTN5_HUMAN (Q9NRC6) Spectrin beta chain, brain 4 (Spectrin, | Q9NRC6 | 416577.2 | 6.09E−03 | 0556 |
| | non-erythroid beta chain 4) (Beta-V sp | | | | |
| 948 | STRN4_HUMAN (Q9NRL3) Striatin-4 (Zinedin) | Q9NRL3 | 80532.22 | 8.80E−03 | 0578 |
| 949 | Q9NRX1 (Q9NRX1) Putatative 28 kDa protein (RNA-binding protein | Q9NRX1 | 27906.86 | 2.17E−03 | 0549 |
| | LOC56902) (Hypothetical protein LOC5 | | | | |
| 950 | Q9NS08 (Q9NS08) HSPC045 protein | Q9NS08 | 23897.4 | 6.08E−03 | 0571 |
| 951 | Q9NSG2 (Q9NSG2) Hypothetical protein C1orf112 (Novel protein) | Q9NSG2 | 96491.85 | 3.39E−03 | 0574 |
| 952 | Q9NSI1 (Q9NSI1) PRED57 protein | Q9NSI1 | 12605.25 | 1.96E−03 | 0560 |
| 953 | MDN1_HUMAN (Q9NU22) Midasin (MIDAS-containing protein) | Q9NU22 | 632425.4 | 5.39E−03 | 0535 |
| 954 | Q9NVD6 (Q9NVD6) Hypothetical protein FLJ10795 | Q9NVD6 | 98613.02 | 6.33E−03 | 0549 |
| 955 | Q9NXB7 (Q9NXB7) Hypothetical protein FLJ20336 | Q9NXB7 | 93451.76 | 8.21E−03 | 0535 |
| 956 | STAB1_HUMAN (Q9NY15) Stabilin-1 precursor (FEEL-1 protein) | Q9NY15 | 275292.3 | 8.37E−03 | 0552 |
| | (MS-1 antigen) | | | | |
| 957 | ZN226_HUMAN (Q9NYT6) Zinc finger protein 226 | Q9NYT6 | 91861.71 | 9.11E−03 | 0571 |
| 958 | NLGN3_HUMAN (Q9NZ94) Neuroligin-3 precursor (Gliotactin | Q9NZ94 | 93836.29 | 7.55E−03 | 0556 |
| | homolog) | | | | |
| 959 | COMD9_HUMAN (Q9P000) COMM domain containing protein 9 | Q9P000 | 21904.53 | 4.92E−03 | 0574 |
| 960 | Q9P0P3 (Q9P0P3) HSPC235 | Q9P0P3 | 40126.53 | 6.80E−03 | 0560 |
| 961 | K1404_HUMAN (Q9P2E3) Protein KIAA1404 | Q9P2E3 | 220083.3 | 6.94E−03 | 0541 |
| 962 | KLHL8_HUMAN (Q9P2G9) Kelch-like protein 8 | Q9P2G9 | 68816.98 | 8.80E−03 | 0549 |
| 963 | KLHL9_HUMAN (Q9P2J3) Kelch-like protein 9 | Q9P2J3 | 69383.3 | 8.75E−03 | 0574 |
| 964 | ATS9_HUMAN (Q9P2N4) ADAMTS-9 precursor (EC 3.4.24.—) (A | Q9P2N4 | 216415.5 | 1.89E−03 | 0549 |
| | disintegrin and metalloproteinase with thro | | | | |
| 965 | SUCB1_HUMAN (Q9P2R7) Succinyl-CoA ligase [ADP-forming] beta- | Q9P2R7 | 50299.33 | 8.61E−03 | 0535 |
| | chain, mitochondrial precursor (EC 6.2. | | | | |
| 966 | Q9P2T3 (Q9P2T3) TU12B1-TY | Q9P2T3 | 55685.21 | 2.42E−03 | 0541 |
| 967 | SPAST_HUMAN (Q9UBP0) Spastin | Q9UBP0 | 67155.37 | 6.36E−03 | 0535 |
| 968 | CYLN2_HUMAN (Q9UDT6) Cytoplasmic linker protein 2 (Cytoplasmic | Q9UDT6 | 115766.9 | 3.27E−04 | 0567 |
| | linker protein 115) (CLIP-115) (Will | | | | |
| 969 | FBX40_HUMAN (Q9UH90) F-box only protein 40 (Muscle disease- | Q9UH90 | 79612.4 | 1.73E−03 | 0552 |
| | related protein) | | | | |
| 970 | Q9UI92 (Q9UI92) Putative WHSC1 protein | Q9UI92 | 69354.6 | 2.65E−04 | 0541 |
| 971 | ZN230_HUMAN (Q9UIE0) Zinc finger protein 230 (Zinc finger protein | Q9UIE0 | 54357.46 | 2.69E−03 | 0535 |
| | FDZF2) | | | | |
| 972 | Q9UKL0 (Q9UKL0) CoREST protein | Q9UKL0 | 52995.61 | 9.45E−03 | 0574 |
| 973 | MYH13_HUMAN (Q9UKX3) Myosin-13 (Myosin heavy chain, skeletal | Q9UKX3 | 223539.2 | 9.78E−03 | 0567 |
| | muscle, extraocular) (MyHC-eo) | | | | |
| 974 | Q9UL88 (Q9UL88) Myosin-reactive immunoglobulin heavy chain | Q9UL88 | 14133.25 | 1.61E−09 | 0556 |
| | variable region (Fragment) | | | | |
| 975 | Q9ULE0 (Q9ULE0) KIAA1280 protein (Fragment) | Q9ULE0 | 115103.3 | 2.01E−04 | 0560 |
| 976 | Q9ULU2 (Q9ULU2) KIAA1127 protein (Fragment) | Q9ULU2 | 238454.6 | 4.48E−03 | 0549 |
| 977 | ZN179_HUMAN (Q9ULXS) Zinc finger protein 179 (Brain finger | Q9ULX5 | 68310.9 | 5.77E−03 | 0567 |
| | protein) (RING finger protein 112) | | | | |
| 978 | GBRT_HUMAN (Q9UN88) Gamma-aminobutyric-acid receptor theta | Q9UN88 | 71975.9 | 3.03E−03 | 0552 |
| | subunit precursor (GABA(A) receptor) | | | | |
| 979 | TRI35_HUMAN (Q9UPQ4) Tripartite motif protein 35 (Hemopoeitic | Q9UPQ4 | 56503.89 | 2.53E−03 | 0571 |
| | lineage switch protein 5) | | | | |
| 980 | CD2L2_HUMAN (Q9UQ88) PITSLRE serine/threonine-protein kinase | Q9UQ88 | 90948.52 | 3.00E−03 | 0567 |
| | CDC2L2 (EC 2.7.1.37) (Galactosyltransf | | | | |
| 981 | MTMR7_HUMAN (Q9Y216) Myotubularin-related protein 7 (EC 3.1.3.—) | Q9Y216 | 45407.51 | 2.59E−03 | 0541 |
| | (Fragment) | | | | |
| 982 | FYV1_HUMAN (Q9Y2I7) FYVE finger-containing phosphoinositide | Q9Y2I7 | 236956.6 | 8.19E−03 | 0541 |
| | kinase (EC 2.7.1.68) (1-phosphatidylino | | | | |
| 983 | Q9Y2K3 (Q9Y2K3) KIAA1000 protein (Fragment) | Q9Y2K3 | 225676.5 | 3.13E−03 | 0571 |
| 984 | SYYM_HUMAN (Q9Y2Z4) Probable tyrosyl-tRNA synthetase, | Q9Y2Z4 | 53165.8 | 5.75E−03 | 0541 |
| | mitochondrial precursor (EC 6.1.1.1) (Tyrosin | | | | |
| 985 | ZN337_HUMAN (Q9Y3M9) Zinc finger protein 337 | Q9Y3M9 | 86819.26 | 9.12E−03 | 0552 |
| 986 | TRRAP_HUMAN (Q9Y4A5) Transformation/transcription domain- | Q9Y4A5 | 437303 | 1.86E−03 | 0560 |
| | associated protein (350/400 kDa PCAF-assoc | | | | |
| 987 | UBP15_HUMAN (Q9Y4E8) Ubiquitin carboxyl-terminal hydrolase 15 | Q9Y4E8 | 112347.6 | 3.17E−03 | 0549 |
| | (EC 3.1.2.15) (Ubiquitin thiolesteras | | | | |
| 988 | M4K5_HUMAN (Q9Y4K4) Mitogen-activated protein kinase kinase | Q9Y4K4 | 94979.4 | 8.66E−03 | 0567 |
| | kinase kinase 5 (EC 2.7.1.37) (MAPK/ERK | | | | |
| 989 | Q9Y528 (Q9Y528) Hypothetical protein (Fragment) | Q9Y528 | 77665.97 | 9.30E−03 | 0560 |
| 990 | PCDBE_HUMAN (Q9Y5E9) Protocadherin beta 14 precursor (PCDH- | Q9Y5E9 | 87494.04 | 1.47E−03 | 0571 |
| | beta14) | | | | |
| 991 | PCDGE_HUMAN (Q9Y5G2) Protocadherin gamma B2 precursor | Q9Y5G2 | 100813.4 | 3.07E−03 | 0567 |
| | (PCDH-gamma-B2) | | | | |
| 992 | PCDG6_HUMAN (Q9Y5G7) Protocadherin gamma A6 precursor | Q9Y5G7 | 100809.6 | 4.10E−03 | 0578 |
| | (PCDH-gamma-A6) | | | | |
| 993 | ST14_HUMAN (Q9Y5Y6) Suppressor of tumorigenicity 14 (EC | Q9Y5Y6 | 94708.81 | 1.92E−03 | 0571 |
| | 3.4.21.—) (Matriptase) (Membrane-type serin | | | | |
| 994 | S12A7_HUMAN (Q9Y666) Solute carrier family 12 member 7 | Q9Y666 | 119072.7 | 5.71E−03 | 0567 |
| | (Electroneutral potassium-chloride cotranspo | | | | |
| 995 | MD1L1_HUMAN (Q9Y6D9) Mitotic spindle assembly checkpoint | Q9Y6D9 | 83015.71 | 4.46E−03 | 0571 |
| | protein MAD1 (Mitotic arrest deficient-lik | | | | |
| 996 | CDY1_HUMAN (Q9Y6F8) Testis-specific chromodomain protein Y 1 | Q9Y6F8 | 60435.05 | 6.22E−03 | 0549 |

TABLE 5
Proteins identified in 25 runs (5 patients with diagnosed Retinal Detachment)
SEQUEST filters: p prob < 0.01; Xcorr vs charge state: 1.5 for [M + H]1+, 2.0 for [M + 2H]2+, 2.5 for [M + 3H]3+; DCn > 0.1

| | | | | | | [symbol] tide | counts | [symbol] tot ids in |
|---|---|---|---|---|---|---|---|---|
| | Patients | Reference | P (pro) | MW | Accession | (Hits) | in R[ [symbol] | 25 runs |
| 1 | BACED | Serum albumin precursor | 1.33E−14 | 69321.63 | P02768 | 60 | 5 | 25 |
| 2 | AEBDC | Serotransferrin precursor | 1.41E−13 | 76999.66 | P02787 | 50 | 5 | 25 |
| 3 | ACDEB | Hypothetical protein | 2.78E−07 | 20655.36 | Q569I7 | 3 | 5 | 22 |
| 4 | ECBAD | Transthyretin precursor | 6.00E−14 | 15877.05 | P02766 | 2 | 5 | 20 |
| 5 | EBDAC | Interphotoreceptor retinoid-binding protein precursor | 1.00E−30 | 135277.8 | P10745 | 1 | 5 | 19 |
| 6 | CADEB | PH domain containing protein | 8.00E−05 | 53317.19 | Q8TD55 | 1 | 5 | 18 |
| 7 | DAECB | Alpha-1-acid glycoprotein 1 precursor | 1.02E−09 | 23496.77 | P02763 | 2 | 5 | 17 |
| 8 | CBDA | Keratin, type II cytoskeletal 1 | 2.94E−11 | 65846.88 | P04264 | 1 | 4 | 17 |
| 9 | ABDEC | Clusterin precursor | 3.33E−12 | 52461.05 | P10909 | 9 | 5 | 16 |
| 10 | AEBDC | Hemopexin precursor | 8.88E−15 | 51643.32 | P02790 | 4 | 5 | 13 |
| 11 | CDEAB | Transcription factor NF-E4 | 3.06E−04 | 19006.73 | Q86UQ8 | 2 | 5 | 13 |
| 12 | AEBD | Apolipoprotein A-I precursor | 3.51E−10 | 30758.94 | P02647 | 8 | 4 | 13 |
| 13 | AECDB | Alpha-1-antitrypsin precursor | 8.88E−15 | 46707.09 | P01009 | 4 | 5 | 12 |
| 14 | AECBD | Nuclear receptor-interacting factor | 2.52E−03 | 52754.03 | Q8WY14 | 1 | 5 | 12 |
| 15 | ABCDE | Ig gamma-1 chain C region | 4.94E−13 | 36083.16 | P01857 | 8 | 5 | 11 |
| 16 | DBC | Alpha 2 globin variant | 3.80E−12 | 15270.94 | Q53F97 | 4 | 3 | 11 |
| 17 | CEAB | Apolipoprotein A-II precursor | 5.95E−12 | 11167.9 | P02652 | 1 | 4 | 10 |
| 18 | ADCE | Hypothetical protein FLJ40259 | 1.29E−04 | 66086.88 | Q8N7W7 | 1 | 4 | 10 |
| 19 | BEA | Pigment epithelium-derived factor precursor | 1.93E−10 | 46313.36 | P36955 | 8 | 3 | 10 |
| 20 | DBA | Keratin, type I cytoskeletal 10 | 1.51E−09 | 59482.76 | P13645 | 1 | 3 | 10 |
| 21 | BD | Hemoglobin delta subunit | 1.78E−14 | 15914.25 | P02042 | 13 | 2 | 10 |
| 22 | BD | Beta-globin gene from a thalassemia patient | 2.17E−12 | 18918.59 | Q14473 | 10 | 2 | 10 |
| 23 | AECB | Hypothetical protein FLJ42076 | 3.47E−04 | 21036.72 | Q6ZVU4 | 2 | 4 | 9 |
| 24 | DCEAB | PML-RARA regulated adaptor molecule 1 | 1.38E−03 | 73950.18 | Q8N6W7 | 1 | 5 | 8 |
| 25 | DAEC | Keratin, type II cytoskeletal 1b | 2.57E−06 | 61650.4 | Q7Z794 | 2 | 4 | 8 |
| 26 | DECA | Double-stranded RNA-binding protein | 1.39E−04 | 63227.92 | O95793 | 1 | 4 | 8 |
| 27 | ECDA | HNRBF-2 | 5.30E−05 | 31787.43 | Q9H2I2 | 1 | 4 | 8 |
| 28 | BCD | Vacuolar protein sorting 13A | 9.97E−04 | 360046.1 | Q96RL7 | 1 | 3 | 8 |
| 29 | AEDCB | Zinc finger protein 185 | 4.35E−03 | 49156.87 | O15231 | 1 | 5 | 7 |
| 30 | ABED | Complement C4-A precursor | 4.22E−14 | 192649.5 | P0C0L4 | 1 | 4 | 7 |
| 31 | CDAE | ADAMTS-12 precursor | 2.30E−04 | 177429.5 | P58397 | 1 | 4 | 7 |
| 32 | BCDA | Keratin 10 | 5.82E−13 | 57212.99 | Q14664 | 1 | 4 | 7 |
| 33 | AB | Dickkopf-related protein 3 precursor | 2.33E−08 | 38266.07 | Q9UBP4 | 3 | 2 | 7 |
| 34 | EDCB | Colony stimulating factor 2 receptor | 7.80E−05 | 46871.63 | Q4V312 | 1 | 4 | 6 |
| 35 | EDCA | KIAA1200 protein | 2.86E−03 | 155607.6 | Q9ULM0 | 1 | 4 | 6 |
| 36 | ACE | Tuberin (Tuberous sclerosis 2 protein) | 7.82E−05 | 200621.4 | P49815 | 1 | 3 | 6 |
| 37 | CAE | Kallikrein-14 precursor | 1.67E−04 | 27434.89 | Q9P0G3 | 1 | 3 | 6 |
| 38 | CBDE | LOC131076 protein | 2.50E−03 | 16609.49 | Q4VC31 | 1 | 4 | 5 |
| 39 | DBCA | Hypothetical protein DKFZp686J11235 | 9.99E−08 | 54424.95 | Q6MZW0 | 1 | 4 | 5 |
| 40 | EBC | Keratin, type II | 5.27E−07 | 54935.82 | Q14533 | 1 | 3 | 5 |
| 41 | ECA | Tumor necrosis factor | 1.04E−03 | 20099.21 | Q5VWH1 | 1 | 3 | 5 |
| 42 | AE | Vitamin D-binding protein | 7.09E−10 | 52902.04 | Q6GTG1 | 4 | 2 | 5 |
| 43 | BA | Keratin, type II cytoskeletal | 5.57E−07 | 65825.37 | P35908 | 1 | 2 | 5 |
| 44 | CA | LOC150763 protein | 7.57E−05 | 87507.18 | Q6NUI2 | 1 | 2 | 5 |
| 45 | EC | Hypothetical protein FLJ44670 | 5.11E−03 | 115755 | Q6ZTG8 | 1 | 2 | 5 |
| 46 | EDCB | Hypothetical protein DKFZp434I138 | 2.57E−04 | 78784.1 | Q659E3 | 1 | 4 | 4 |
| 47 | CEA | Histone acetyltransferase | 6.68E−04 | 92953.77 | Q92831 | 1 | 3 | 4 |
| 48 | DCB | WD-repeat protein 9 | 2.64E−03 | 257060.4 | Q9NSI6 | 1 | 3 | 4 |
| 49 | AB | Hypothetical protein DKFZp686I04196 | 1.25E−11 | 46031.66 | Q6N093 | 6 | 2 | 4 |
| 50 | EC | Proto-oncogene tyrosine-protein kinase | 4.63E−05 | 93412.31 | P07332 | 1 | 2 | 4 |
| 51 | AB | Cathepsin D precursor | 3.66E−09 | 44523.66 | P07339 | 1 | 2 | 4 |
| 52 | AE | Cytochrome P450 | 4.19E−04 | 55766.2 | P10635 | 1 | 2 | 4 |
| 53 | BD | Peroxiredoxin 1 | 1.57E−06 | 22096.28 | Q06830 | 1 | 2 | 4 |
| 54 | ED | Hypothetical protein FLJ43007 | 1.02E−04 | 19332.49 | Q6ZV44 | 1 | 2 | 4 |
| 55 | CE | Hypothetical protein FLJ20202 | 2.74E−03 | 44899.86 | Q9NXK0 | 1 | 2 | 4 |
| 56 | BAE | Ceruloplasmin precursor | 1.11E−12 | 122127.6 | P00450 | 3 | 3 | 3 |
| 57 | ABD | Complement C3 precursor | 5.42E−11 | 187045.3 | P01024 | 1 | 3 | 3 |
| 58 | ABE | Intercellular adhesion molecule 3 precursor | 4.62E−03 | 59345.85 | P32942 | 1 | 3 | 3 |
| 59 | ECD | Nuclear pore complex protein Nup214 | 4.38E−03 | 213632.8 | P35658 | 1 | 3 | 3 |
| 60 | EAD | Dystroglycan precursor | 1.78E−03 | 97519.91 | Q14118 | 1 | 3 | 3 |
| 61 | ADC | Alpha-internexin | 2.07E−03 | 55357.48 | Q16352 | 1 | 3 | 3 |
| 62 | ECA | Transmembrane channel-like protein 7 | 2.53E−03 | 83447.49 | Q7Z402 | 1 | 3 | 3 |
| 63 | BCE | Hypothetical protein FLJ40243 | 1.50E−03 | 180566.4 | Q7Z745 | 1 | 3 | 3 |
| 64 | DEA | T-cell activation Rho GTPase-activating protein | 5.60E−03 | 80652.9 | Q8N103 | 1 | 3 | 3 |
| 65 | BCA | Hypothetical protein FLJ40479 | 3.57E−03 | 45893.4 | Q8N7Q3 | 1 | 3 | 3 |
| 66 | EDC | Protein C9orf126 | 1.82E−03 | 70385.07 | Q8N9R8 | 1 | 3 | 3 |
| 67 | ACB | Hypothetical protein FLJ35435 | 1.05E−04 | 65308.66 | Q8NAF6 | 1 | 3 | 3 |
| 68 | BAD | Protein KIAA1199 precursor | 1.11E−03 | 152900 | Q8WUJ3 | 1 | 3 | 3 |
| 69 | CDE | G patch domain containing protein 3 | 2.28E−03 | 59301.11 | Q96I76 | 1 | 3 | 3 |
| 70 | BA | Alpha-2-macroglobulin precursor | 9.36E−09 | 163174.3 | P01023 | 2 | 2 | 3 |
| 71 | BA | Ig gamma-3 chain C region | 1.07E−08 | 32309.81 | P01860 | 2 | 2 | 3 |
| 72 | CE | Basement membrane-specific heparan sulfate proteoglyca [symbol] | 4.04E−04 | 468528.2 | P98160 | 1 | 2 | 3 |
| 73 | CA | Trichohyalin | 2.97E−03 | 247074.4 | Q07283 | 1 | 2 | 3 |
| 74 | BA | IGLC2 protein | 2.41E−11 | 24784.13 | Q567P1 | 1 | 2 | 3 |
| 75 | BE | Receptor interacting protein kinase 5 | 2.01E−03 | 99957.35 | Q5RKT0 | 1 | 2 | 3 |
| 76 | BD | Novel protein | 2.74E−04 | 91277.53 | Q5VVM6 | 1 | 2 | 3 |
| 77 | CD | Hypothetical protein | 5.22E−03 | 9188.304 | Q6PHR9 | 1 | 2 | 3 |
| 78 | AC | Hypothetical protein FAM33A | 4.24E−03 | 14179.33 | Q8WVK7 | 1 | 2 | 3 |
| 79 | CA | Hypothetical protein FLJ22346 | 3.68E−03 | 81825.66 | Q9H6E6 | 1 | 2 | 3 |
| 80 | EC | Calcium-independent phospholipase A2 | 2.83E−03 | 88421.11 | Q9NP80 | 1 | 2 | 3 |
| 81 | DE | Endocrine regulator | 4.94E−04 | 231761.3 | Q9Y2W9 | 1 | 2 | 3 |
| 82 | E | Potassium voltage-gated channel subfamily A | 8.04E−04 | 56505.6 | Q09470 | 1 | 1 | 3 |
| 83 | D | Huntingtin interacting protein E | 7.33E−05 | 51745.16 | Q9BVA6 | 1 | 1 | 3 |
| 84 | BE | KIAA0068 protein (Fragment) | 2.65E−04 | 147040.6 | Q14467 | 2 | 2 | 2 |
| 85 | AD | Protein C19orf10 precursor (Stromal cell-derived GF) | 6.67E−03 | 18783.32 | Q969H8 | 2 | 2 | 2 |
| 86 | CE | Hypothetical protein FLJ21129 | 2.87E−03 | 63276.44 | Q9H796 | 2 | 2 | 2 |
| 87 | CE | HS1-associating protein X-1 | 2.63E−03 | 31601.06 | O00165 | 1 | 2 | 2 |
| 88 | AD | Tumor necrosis factor receptor superfamily | 7.09E−03 | 50028.81 | O00220 | 1 | 2 | 2 |
| 89 | AB | Zinc finger protein 197 | 4.82E−03 | 118770.8 | O14709 | 1 | 2 | 2 |
| 90 | EA | Inositol polyphosphate 5-phosphatase | 1.69E−04 | 138498.9 | O15357 | 1 | 2 | 2 |
| 91 | DE | DNA repair protein RAD51 homolog 3 | 3.18E−03 | 42162.89 | O43502 | 1 | 2 | 2 |
| 92 | DA | TBC1 domain family member 4 (Akt substrate) | 7.09E−04 | 146470.8 | O60343 | 1 | 2 | 2 |
| 93 | EC | Zinc finger protein ZIC 2 | 3.64E−03 | 54971.09 | O95409 | 1 | 2 | 2 |
| 94 | BA | Supervillin | 5.54E−03 | 247550.6 | O95425 | 1 | 2 | 2 |
| 95 | CA | Ig lambda chain V region | 4.35E−03 | 12372.02 | P04211 | 1 | 2 | 2 |
| 96 | BE | Keratin, type II | 4.19E−05 | 59831.25 | P04259 | 1 | 2 | 2 |
| 97 | AB | Osteopontin precursor | 3.06E−07 | 35401.25 | P10451 | 1 | 2 | 2 |
| 98 | CA | Cation-independent mannose-6-phosphate receptor | 5.66E−03 | 274097.6 | P11717 | 1 | 2 | 2 |
| 99 | DE | Filamin-A | 3.86E−03 | 280583.4 | P21333 | 1 | 2 | 2 |
| 100 | CE | Macrophage scavenger receptor types I and II | 7.76E−04 | 49730.82 | P21757 | 1 | 2 | 2 |
| 101 | EC | Receptor-type tyrosine-protein phosphatase delta | 3.04E−03 | 214623.5 | P23468 | 1 | 2 | 2 |
| 102 | EA | Epoxide hydrolase 2 | 1.17E−03 | 62574.77 | P34913 | 1 | 2 | 2 |
| 103 | AB | Prostaglandin-H2 D-isomerase | 3.34E−10 | 21015.35 | P41222 | 1 | 2 | 2 |
| 104 | CD | Matrin-3 | 3.80E−03 | 94564.67 | P43243 | 1 | 2 | 2 |
| 105 | CE | Voltage-dependent anion-selective channel protein 2 | 4.14E−04 | 38068.68 | P45880 | 1 | 2 | 2 |
| 106 | ED | G protein-activated inward rectifier potassium channel 1 | 4.27E−04 | 56566.8 | P48549 | 1 | 2 | 2 |
| 107 | BA | Dual specificity protein phosphatase 5 | 2.53E−04 | 42080.39 | Q16690 | 1 | 2 | 2 |
| 108 | DE | Maguin-like protein variant II | 1.71E−03 | 61848.88 | Q5SGD5 | 1 | 2 | 2 |
| 109 | AB | RhoGTPase regulating protein variant ARHGAP20-1be | 7.13E−03 | 130153.4 | Q6RJU1 | 1 | 2 | 2 |
| 110 | BE | FLJ00268 protein (Fragment) | 2.78E−03 | 72805.3 | Q6ZML5 | 1 | 2 | 2 |
| 111 | BD | Hypothetical protein FLJ44832 | 4.10E−03 | 139335.1 | Q6ZQT3 | 1 | 2 | 2 |
| 112 | CE | RUN domain containing 1 | 2.55E−03 | 67603.02 | Q8IXT9 | 1 | 2 | 2 |
| 113 | BD | Hypothetical protein TMEM55A | 4.69E−05 | 28062.26 | Q8N4L2 | 1 | 2 | 2 |
| 114 | ED | Hypothetical protein DKFZp547B1713 | 9.06E−03 | 32732.49 | Q8NDD1 | 1 | 2 | 2 |
| 115 | CE | GCN1-like protein 1 (HsGCN1) | 5.09E−03 | 292424.8 | Q92616 | 1 | 2 | 2 |
| 116 | CA | LAG1 longevity assurance homolog 2 | 1.46E−04 | 44847.38 | Q96G23 | 1 | 2 | 2 |
| 117 | CA | Serine/threonine-protein kinase | 8.12E−03 | 134655.6 | Q96J92 | 1 | 2 | 2 |
| 118 | DB | Hypothetical protein gs80 | 9.47E−04 | 25483.99 | Q96S02 | 1 | 2 | 2 |
| 119 | CD | Hypothetical protein FLJ13940 | 2.89E−03 | 27528.79 | Q9H853 | 1 | 2 | 2 |
| 120 | CD | ADAM 28 precursor | 2.62E−04 | 87151.09 | Q9UKQ2 | 1 | 2 | 2 |
| 121 | EB | KIAA1238 protein (Fragment) | 7.85E−03 | 45141.04 | Q9ULI2 | 1 | 2 | 2 |
| 122 | AC | Mitochondrial 28S ribosomal protein S2 | 5.60E−05 | 33228.08 | Q9Y399 | 1 | 2 | 2 |
| 123 | A | IGKC protein | 1.11E−15 | 25919.92 | Q502W4 | 3 | 1 | 2 |
| 124 | B | Adenylate kinase isoenzyme 2 | 1.02E−03 | 26329.73 | P54819 | 2 | 1 | 2 |
| 125 | B | AACT_HUMAN (P01011) Alpha-1-antichymotrypsin precur [symbol] | 2.48E−04 | 47620.63 | P01011 | 1 | 1 | 2 |
| 126 | C | DAX1_HUMAN (P51843) Nuclear receptor 0B1 (Nuclear r [symbol] | 1.40E−03 | 51683.89 | P51843 | 1 | 1 | 2 |
| 127 | C | Q684R2 (Q684R2) Para-hydroxybenzoate-polyprenyltrans [symbol] | 3.25E−03 | 45578.79 | Q684R2 | 1 | 1 | 2 |
| 128 | B | Q6PHR8 (Q6PHR8) Hypothetical protein (Fragment) | 6.89E−03 | 33452.8 | Q6PHR8 | 1 | 1 | 2 |
| 129 | E | Q8IYE0 (Q8IYE0) Hypothetical protein KIAA1505 | 3.71E−03 | 112734.9 | Q8IYE0 | 1 | 1 | 2 |
| 130 | D | ZN473_HUMAN (Q8WTR7) Zinc finger protein 473 (Zinc fi [symbol] | 2.47E−03 | 100118.1 | Q8WTR7 | 1 | 1 | 2 |
| 131 | B | CPSF3_HUMAN (Q9UKF6) Cleavage and polyadenylation [symbol] | 1.14E−03 | 77436.22 | Q9UKF6 | 1 | 1 | 2 |
| 132 | B | K2C5_HUMAN (P13647) Keratin, type II cytoskeletal 5 (Cy [symbol] | 7.34E−08 | 62409.08 | P13647 | 3 | 1 | 1 |
| 133 | E | TLR5_HUMAN (O60602) Toll-like receptor 5 precursor (To [symbol] | 3.64E−03 | 97663.38 | O60602 | 2 | 1 | 1 |
| 134 | B | CAH1_HUMAN (P00915) Carbonic anhydrase 1 (EC 4.2.1. [symbol] | 8.52E−08 | 28721.34 | P00915 | 2 | 1 | 1 |
| 135 | A | CYTC_HUMAN (P01034) Cystatin C precursor (Neuroend [symbol] | 1.70E−04 | 15789.08 | P01034 | 2 | 1 | 1 |
| 136 | A | KNG1_HUMAN (P01042) Kininogen-1 precursor (Alpha-2- [symbol] | 2.12E−10 | 71900.1 | P01042 | 2 | 1 | 1 |
| 137 | B | K1C14_HUMAN (P02533) Keratin, type I cytoskeletal 14 ( [symbol] | 3.33E−06 | 51458.45 | P02533 | 2 | 1 | 1 |
| 138 | B | APOE_HUMAN (P02649) Apolipoprotein E precursor (Apo [symbol] | 3.07E−03 | 36131.79 | P02649 | 2 | 1 | 1 |
| 139 | B | K1C9_HUMAN (P35527) Keratin, type I cytoskeletal 9 (Cyt [symbol] | 2.20E−12 | 62091.76 | P35527 | 2 | 1 | 1 |
| 140 | D | O00211 (O00211) HSLK (Ste20-related serineVthreonine k [symbol] | 8.39E−03 | 138909.7 | O00211 | 1 | 1 | 1 |
| 141 | D | P3C2A_HUMAN (O00443) Phosphatidylinositol-4-phospha [symbol] | 7.07E−03 | 190616.2 | O00443 | 1 | 1 | 1 |
| 142 | B | SYT5 HUMAN (O00445) Synaptotagmin = 5 (Synaptotagmi [symbol] | 3.71E−03 | 42873.54 | O00445 | 1 | 1 | 1 |
| 143 | A | CHD2_HUMAN (O14647) Chromodomain-helicase-DNA-b [symbol] | 7.04E−03 | 200437.6 | O14647 | 1 | 1 | 1 |
| 144 | C | MLL2_HUMAN (O14686) Myeloid/lymphoid or mixed-linea [symbol] | 8.21E−04 | 563835.8 | O14686 | 1 | 1 | 1 |
| 145 | E | O15042 (O15042) Hypothetical protein KIAA0332 (U2-ass [symbol] | 7.16E−04 | 118087.4 | O15042 | 1 | 1 | 1 |
| 146 | B | SNPH_HUMAN (O15079) Syntaphilin | 7.58E−03 | 57953.73 | O15079 | 1 | 1 | 1 |
| 147 | A | WDR46_HUMAN (O15213) WD-repeat protein 46 (WD-re [symbol] | 3.67E−03 | 67998.89 | O15213 | 1 | 1 | 1 |
| 148 | A | SGCE_HUMAN (O43556) Epsilon-sarcoglycan precursor ( [symbol] | 2.18E−04 | 49722.25 | O43556 | 1 | 1 | 1 |
| 149 | E | HAPIP_HUMAN (O60229) Huntingtin-associated protein-in [symbol] | 3.37E−03 | 192107.9 | O60229 | 1 | 1 | 1 |
| 150 | E | O60687 (O60687) Sushi-repeat-containing protein, X-linke [symbol] | 4.07E−03 | 52937.79 | O60687 | 1 | 1 | 1 |
| 151 | D | ZC11A_HUMAN (O75152) Zinc finger CCCH-type domain | 4.33E−03 | 89076.06 | O75152 | 1 | 1 | 1 |
| 152 | C | O75198 (O75198) CDC37-like gene | 1.49E−03 | 26530.93 | O75198 | 1 | 1 | 1 |
| 153 | A | AVIL_HUMAN (O75366) Advillin (p92) | 8.01E−03 | 92028.35 | O75366 | 1 | 1 | 1 |
| 154 | C | MYCB2_HUMAN (O75592) Probable ubiquitin ligase prote [symbol] | 7.36E−03 | 509842.3 | O75592 | 1 | 1 | 1 |
| 155 | A | CMC1_HUMAN (O75746) Calcium-binding mitochondrial [symbol] | 3.32E−03 | 74709.01 | O75746 | 1 | 1 | 1 |
| 156 | E | MFN2_HUMAN (O95140) Transmembrane GTPase MFN2 [symbol] | 4.18E−03 | 86346.95 | O95140 | 1 | 1 | 1 |
| 157 | B | G6PE_HUMAN (O95479) GDH/6PGL endoplasmic bifunct [symbol] | 3.69E−03 | 88822.63 | O95479 | 1 | 1 | 1 |
| 158 | A | O95978 (O95978) VH1 protein precursor (Fragment) | 1.17E−07 | 17292.5 | O95978 | 1 | 1 | 1 |
| 159 | D | HPTR_HUMAN (P00739) Haptoglobin-related protein prec [symbol] | 5.05E−03 | 38982.66 | P00739 | 1 | 1 | 1 |
| 160 | A | CFAB_HUMAN (P00751) Complement factor B precursor [symbol] | 2.24E−04 | 85478.58 | P00751 | 1 | 1 | 1 |
| 161 | A | ASSY_HUMAN (P00966) Argininosuccinate synthase (EC [symbol] | 4.02E−03 | 46501.01 | P00966 | 1 | 1 | 1 |
| 162 | B | K2C6A_HUMAN (P02538) Keratin, type II cytoskeletal 6A ( [symbol] | 4.96E−03 | 59877.28 | P02538 | 1 | 1 | 1 |
| 163 | B | APOH_HUMAN (P02749) Beta-2-glycoprotein I precursor ( [symbol] | 3.96E−03 | 38272.67 | P02749 | 1 | 1 | 1 |
| 164 | A | RETBP HUMAN (P02753) Plasma retinol = binding protein [symbol] | 1.36E−03 | 22995.26 | P02753 | 1 | 1 | 1 |
| 165 | A | FETUA_HUMAN (P02765) Alpha-2-HS-glycoprotein precu [symbol] | 3.37E−04 | 39299.73 | P02765 | 1 | 1 | 1 |
| 166 | B | VTNC_HUMAN (P04004) Vitronectin precursor (Serum sp [symbol] | 1.09E−03 | 54271.23 | P04004 | 1 | 1 | 1 |
| 167 | B | HRG_HUMAN (P04196) Histidine-rich glycoprotein precur [symbol] | 5.94E−04 | 59540.94 | P04196 | 1 | 1 | 1 |
| 168 | A | CFAI_HUMAN (P05156) Complement factor I precursor (E [symbol] | 2.93E−05 | 65676.66 | P05156 | 1 | 1 | 1 |
| 169 | D | ITB1_HUMAN (P05556) Integrin beta-1 precursor (Fibrone [symbol] | 7.50E−05 | 88406.98 | P05556 | 1 | 1 | 1 |
| 170 | A | KV2F_HUMAN (P06310) Ig kappa chain V-II region RPMI [symbol] | 1.78E−06 | 14697.37 | P06310 | 1 | 1 | 1 |
| 171 | B | GELS_HUMAN (P06396) Gelsolin precursor (Actin-depoly [symbol] | 5.71E−04 | 85644.25 | P06396 | 1 | 1 | 1 |
| 172 | B | K1C16_HUMAN (P08779) Keratin, type I cytoskeletal 16 ( [symbol] | 1.08E−06 | 51105.21 | P08779 | 1 | 1 | 1 |
| 173 | E | TAU_HUMAN (P10636) Microtubule-associated protein ta [symbol] | 7.47E−04 | 78698.83 | P10636 | 1 | 1 | 1 |
| 174 | D | PABP1_HUMAN (P11940) Polyadenylate-binding protein 1 [symbol] | 9.99E−03 | 70626.04 | P11940 | 1 | 1 | 1 |
| 175 | B | SCG2_HUMAN (P13521) Secretogranin-2 precursor (Secr [symbol] | 6.93E−03 | 70825.11 | P13521 | 1 | 1 | 1 |
| 176 | C | UCHL3_HUMAN (P15374) Ubiquitin carboxyl-terminal hyd [symbol] | 6.36E−03 | 26165.98 | P15374 | 1 | 1 | 1 |
| 177 | C | PDE6A_HUMAN (P16499) Rod cGMP-specific 3′,5′-cyclic [symbol] | 4.04E−03 | 99438.76 | P16499 | 1 | 1 | 1 |
| 178 | B | CBPE_HUMAN (P16870) Carboxypeptidase E precursor ( [symbol] | 1.65E−06 | 53117.17 | P16870 | 1 | 1 | 1 |
| 179 | B | AATC_HUMAN (P17174) Aspartate aminotransferase, cyt [symbol] | 9.28E−03 | 46087.54 | P17174 | 1 | 1 | 1 |
| 180 | B | A1AG2_HUMAN (P19652) Alpha-1-acid glycoprotein 2 pre [symbol] | 4.46E−06 | 23587.64 | P19652 | 1 | 1 | 1 |
| 181 | B | A1ATR_HUMAN (P20848) Alpha-1-antitrypsin-related prot [symbol] | 7.19E−03 | 47860.97 | P20848 | 1 | 1 | 1 |
| 182 | E | RAE2_HUMAN (P26374) Rab proteins geranylgeranyltrans [symbol] | 1.83E−03 | 74024.21 | P26374 | 1 | 1 | 1 |
| 183 | A | MRP1_HUMAN (P33527) Multidrug resistance-associated [symbol] | 1.77E−03 | 171450.5 | P33527 | 1 | 1 | 1 |
| 184 | A | TSP2_HUMAN (P35442) Thrombospondin-2 precursor | 3.33E−04 | 129871.5 | P35442 | 1 | 1 | 1 |
| 185 | B | VGFR2_HUMAN (P35968) Vascular endothelial growth fa [symbol] | 1.56E−03 | 151430.5 | P35968 | 1 | 1 | 1 |
| 186 | B | CH3L1_HUMAN (P36222) Chitinase-3-like protein 1 precu [symbol] | 2.71E−06 | 42586.39 | P36222 | 1 | 1 | 1 |
| 187 | B | MAP1B_HUMAN (P46821) Microtubule-associated protein [symbol] | 5.22E−03 | 270451.8 | P46821 | 1 | 1 | 1 |
| 188 | B | PCY1A_HUMAN (P49585) Choline-phosphate cytidylyltran [symbol] | 3.65E−03 | 41705.8 | P49585 | 1 | 1 | 1 |
| 189 | E | CEBPD_HUMAN (P49716) CCAAT/enhancer binding prote [symbol] | 6.61E−03 | 28461.45 | P49716 | 1 | 1 | 1 |
| 190 | A | TCPQ_HUMAN (P50990) T-complex protein 1, theta subu [symbol] | 2.31E−03 | 59451.57 | P50990 | 1 | 1 | 1 |
| 191 | D | CCR6_HUMAN (P51684) C—C chemokine receptor type 6 [symbol] | 7.26E−03 | 42465.93 | P51684 | 1 | 1 | 1 |
| 192 | C | EPHA5_HUMAN (P54756) Ephrin type-A receptor 5 precu [symbol] | 7.74E−03 | 114710.3 | P54756 | 1 | 1 | 1 |
| 193 | A | PSD4_HUMAN (P55036) 26S proteasome non-ATPase re [symbol] | 6.62E−04 | 40711.21 | P55036 | 1 | 1 | 1 |
| 194 | A | PSME3_HUMAN (P61289) Proteasome activator complex [symbol] | 3.25E−03 | 29487.55 | P61289 | 1 | 1 | 1 |
| 195 | B | B2MG_HUMAN (P61769) Beta-2-microglobulin precursor [ | 3.51E−07 | 13705.91 | P61769 | 1 | 1 | 1 |
| 196 | A | GBB1_HUMAN (P62873) Guanine nucleotide-binding prot [symbol] | 1.27E−03 | 37221.98 | P62873 | 1 | 1 | 1 |
| 197 | D | CXAR_HUMAN (P78310) Coxsackievirus and adenovirus [symbol] | 1.03E−03 | 40004.39 | P78310 | 1 | 1 | 1 |
| 198 | B | K22O_HUMAN (Q01546) Keratin, type II cytoskeletal 2 ora [symbol] | 8.00E−03 | 65830.09 | Q01546 | 1 | 1 | 1 |
| 199 | A | GALT1_HUMAN (Q10472) Polypeptide N-acetylgalactosar [symbol] | 3.87E−03 | 64177.48 | Q10472 | 1 | 1 | 1 |
| 200 | B | PTN13_HUMAN (Q12923) Tyrosine-protein phosphatase, | 3.13E−05 | 276731.3 | Q12923 | 1 | 1 | 1 |
| 201 | C | DLG1_HUMAN (Q12959) Presynaptic protein SAP97 (Syn [symbol] | 6.56E−03 | 100293.1 | Q12959 | 1 | 1 | 1 |
| 202 | A | AKAP6_HUMAN (Q13023) A-kinase anchor protein 6 (Pro [symbol] | 1.63E−03 | 256503.6 | Q13023 | 1 | 1 | 1 |
| 203 | A | ATM_HUMAN (Q13315) Serine-protein kinase ATM (EC 2. [symbol] | 4.51E−03 | 350417.1 | Q13315 | 1 | 1 | 1 |
| 204 | E | MYO7A_HUMAN (Q13402) Myosin-7A (Myosin VIIa) | 8.23E−04 | 254242.5 | Q13402 | 1 | 1 | 1 |
| 205 | B | ROCK1_HUMAN (Q13464) Rho-associated protein kinase [symbol] | 1.42E−03 | 158074.9 | Q13464 | 1 | 1 | 1 |
| 206 | A | ENPP2_HUMAN (Q13822) Ectonucleotide pyrophosphata [symbol] | 5.80E−10 | 98939.61 | Q13822 | 1 | 1 | 1 |
| 207 | A | UBE4A_HUMAN (Q14139) Ubiquitin conjugation factor E4 [symbol] | 7.03E−03 | 122482 | Q14139 | 1 | 1 | 1 |
| 208 | D | SMC1A_HUMAN (Q14683) Structural maintenance of chr [symbol] | 9.76E−04 | 143143.6 | Q14683 | 1 | 1 | 1 |
| 209 | C | NOLC1_HUMAN (Q14978) Nucleolar phosphoprotein p13 [symbol] | 1.80E−03 | 73676.72 | Q14978 | 1 | 1 | 1 |
| 210 | A | PLEC1_HUMAN (Q15149) Plectin 1 (PLTN) (PCN) (Hemi [symbol] | 2.61E−04 | 531411.9 | Q15149 | 1 | 1 | 1 |
| 211 | E | Q15299 (Q15299) RARB protein | 7.32E−03 | 11644 | Q15299 | 1 | 1 | 1 |
| 212 | E | K1H1_HUMAN (Q15323) Keratin, type I cuticular Ha1 (Hai [symbol] | 1.36E−03 | 47202.1 | Q15323 | 1 | 1 | 1 |
| 213 | A | NCOA1_HUMAN (Q15788) Nuclear receptor coactivator 1 [symbol] | 4.14E−04 | 156641.7 | Q15788 | 1 | 1 | 1 |
| 214 | A | Q15813 (Q15813) Beta-tubulin cofactor E (Tubulin-specific [symbol] | 5.90E−03 | 59309.08 | Q15813 | 1 | 1 | 1 |
| 215 | A | SCN9A_HUMAN (Q15858) Sodium channel protein type I [symbol] | 6.73E−03 | 226193.9 | Q15858 | 1 | 1 | 1 |
| 216 | B | Q495C9 (Q495C9) Hypothetical protein LOC149134 | 6.59E−03 | 13623.18 | Q495C9 | 1 | 1 | 1 |
| 217 | A | Q4G0M1 (Q4G0M1) FLJ37034 protein (Fragment) | 6.80E−03 | 14453.34 | Q4G0M1 | 1 | 1 | 1 |
| 218 | D | Q567P4 (Q567P4) Hypothetical protein (Fragment) | 2.58E−03 | 34603.27 | Q567P4 | 1 | 1 | 1 |
| 219 | A | LRC10_HUMAN (Q5BKY1) Leucine-rich repeat-containing [symbol] | 9.03E−04 | 31621.81 | Q5BKY1 | 1 | 1 | 1 |
| 220 | B | Q5H9K7 (Q5H9K7) Novel protein (Fragment) | 6.32E−03 | 38685.59 | Q5H9K7 | 1 | 1 | 1 |
| 221 | D | Q5H9K8 (Q5H9K8) Armadillo repeat containing, X-linked 4 [symbol] | 7.72E−03 | 36484.3 | Q5H9K8 | 1 | 1 | 1 |
| 222 | C | Q5JRL1 (Q5JRL1) Novel protein | 6.73E−03 | 30747.36 | Q5JRL1 | 1 | 1 | 1 |
| 223 | E | Q5T364 (Q5T364) OTTHUMP00000022702 | 4.05E−03 | 101941 | Q5T364 | 1 | 1 | 1 |
| 224 | C | Q5T3B9 (Q5T3B9) Novel protein | 4.46E−03 | 45993.33 | Q5T3B9 | 1 | 1 | 1 |
| 225 | E | Q5T7N3 (Q5T7N3) Novel protein | 9.47E−03 | 107275.7 | Q5T7N3 | 1 | 1 | 1 |
| 226 | D | Q5TBP2 (Q5TBP2) Retinoblastoma-associated factor 600 [symbol] | 4.66E−03 | 106303.1 | Q5TBP2 | 1 | 1 | 1 |
| 227 | B | Q5VWL1 (Q5VWL1) Membrane-associated guanylate kina [symbol] | 7.51E−03 | 162847.8 | Q5VWL1 | 1 | 1 | 1 |
| 228 | C | Q5XLJ0 (Q5XLJ0) AIDA1C transcript variant 4 | 9.99E−03 | 48400.84 | Q5XLJ0 | 1 | 1 | 1 |
| 229 | E | Q6DN23 (Q6DN23) Platelet phospholipase A2 | 4.11E−03 | 16135.78 | Q6DN23 | 1 | 1 | 1 |
| 230 | C | Q6IAM5 (Q6IAM5) SLC27A6 protein | 3.12E−03 | 70042.55 | Q6IAM5 | 1 | 1 | 1 |
| 231 | B | Q6JIC5 (Q6JIC5) Cementum attachment protein | 9.87E−03 | 14910.42 | Q6JIC5 | 1 | 1 | 1 |
| 232 | A | Q6MZX7 (Q6MZX7) Hypothetical protein DKFZp686M242 [symbol] | 1.00E−30 | 52387.13 | Q6MZX7 | 1 | 1 | 1 |
| 233 | D | Q6P2C5 (Q6P2C5) PHF2 protein | 5.50E−03 | 35720.63 | Q6P2C5 | 1 | 1 | 1 |
| 234 | E | Q6P528 (Q6P528) ASPN protein | 8.71E−03 | 43863.48 | Q6P528 | 1 | 1 | 1 |
| 235 | E | POTE2_HUMAN (Q6S8J3) Prostate, ovary, testis express [symbol] | 6.93E−03 | 80701.5 | Q6S8J3 | 1 | 1 | 1 |
| 236 | E | Q6UWG9 (Q6UWG9) HSAL5836 | 6.47E−03 | 9682.161 | Q6UWG9 | 1 | 1 | 1 |
| 237 | E | Q6UXZ8 (Q6UXZ8) FVSY9334 | 7.07E−04 | 24230.68 | Q6UXZ8 | 1 | 1 | 1 |
| 238 | A | Q6ZN08 (Q6ZN08) Hypothetical protein FLJ16537 | 4.37E−04 | 38813.18 | Q6ZN08 | 1 | 1 | 1 |
| 239 | D | Q6ZR14 (Q6ZR14) Hypothetical protein FLJ46736 | 8.79E−03 | 126662.9 | Q6ZR14 | 1 | 1 | 1 |
| 240 | E | Q6ZSK5 (Q6ZSK5) Hypothetical protein FLJ45433 | 6.04E−03 | 15222.69 | Q6ZSK5 | 1 | 1 | 1 |
| 241 | B | Q6ZSY5 (Q6ZSY5) Hypothetical protein FLJ45123 | 7.90E−03 | 42154.05 | Q6ZSY5 | 1 | 1 | 1 |
| 242 | A | Q6ZVQ3 (Q6ZVQ3) Hypothetical protein FLJ42220 | 1.14E−03 | 17595.21 | Q6ZVQ3 | 1 | 1 | 1 |
| 243 | D | Q6ZW49 (Q6ZW49) Hypothetical protein FLJ41606 | 3.89E−03 | 117615.6 | Q6ZW49 | 1 | 1 | 1 |
| 244 | E | Q702P2 (Q702P2) VPS13C-2B protein | 9.67E−03 | 407940.5 | Q702P2 | 1 | 1 | 1 |
| 245 | C | Q70AK8 (Q70AK8) Ankyrin-repeat-ARM domain protein (F [symbol] | 8.67E−04 | 40496.33 | Q70AK8 | 1 | 1 | 1 |
| 246 | A | Q70CQ2 (Q70CQ2) Ubiquitin-specific proteinase 34 | 9.41E−03 | 386873.7 | Q70CQ2 | 1 | 1 | 1 |
| 247 | C | Q76E79 (Q76E79) Mitochondrial methionyl-tRNA syntheta [symbol] | 2.95E−03 | 66584.84 | Q76E79 | 1 | 1 | 1 |
| 248 | C | Q7KZ97 (Q7KZ97) Antithrombin III variant | 2.00E−03 | 52657.96 | Q7KZ97 | 1 | 1 | 1 |
| 249 | C | Q7L7Q2 (Q7L7Q2) BLOM7 beta (Novel protein) | 1.49E−04 | 59101.39 | Q7L7Q2 | 1 | 1 | 1 |
| 250 | B | Q7RTV7 (Q7RTV7) Adenylyl cyclase type V (Fragment) | 2.76E−04 | 39312.25 | Q7RTV7 | 1 | 1 | 1 |
| 251 | A | TRPM8_HUMAN (Q7Z2W7) Transient receptor potential c [symbol] | 6.02E−03 | 127573.3 | Q7Z2W7 | 1 | 1 | 1 |
| 252 | D | Q7Z3P5 (Q7Z3P5) Hypothetical protein DKFZp686P13218 [symbol] | 5.90E−04 | 30940.46 | Q7Z3P5 | 1 | 1 | 1 |
| 253 | A | NPHP3_HUMAN (Q7Z494) Nephrocystin-3 | 5.74E−03 | 150768.9 | Q7Z494 | 1 | 1 | 1 |
| 254 | E | Q86VG6 (Q86VG6) Hypothetical protein | 3.43E−03 | 11523.95 | Q86VG6 | 1 | 1 | 1 |
| 255 | C | TXND2_HUMAN (Q86VQ3) Thioredoxin domain-containing [symbol] | 7.89E−04 | 60424.09 | Q86VQ3 | 1 | 1 | 1 |
| 256 | D | NAL14_HUMAN (Q86W24) NACHT-, LRR- and PYD-conta [symbol] | 1.88E−03 | 124651.5 | Q86W24 | 1 | 1 | 1 |
| 257 | E | Q86WI3 (Q86WI3) NOD27 | 5.53E−03 | 204535 | Q86WI3 | 1 | 1 | 1 |
| 258 | E | Q86WW8 (Q86WW8) Hypothetical protein MGC52110 (H [symbol] | 4.59E−03 | 8370.071 | Q86WW8 | 1 | 1 | 1 |
| 259 | D | Q86Y92 (Q86Y92) Similar to KIAA0922 protein (Fragment) [symbol] | 2.24E−03 | 154618.6 | Q86Y92 | 1 | 1 | 1 |
| 260 | D | Q8IV73 (Q8IV73) Guanine nucleotide-releasing factor 2, is [symbol] | 3.40E−03 | 122658.6 | Q8IV73 | 1 | 1 | 1 |
| 261 | B | Q8IVL1 (Q8IVL1) Steerin2 protein | 2.98E−03 | 267950.3 | Q8IVL1 | 1 | 1 | 1 |
| 262 | B | Q8IVM4 (Q8IVM4) Hypothetical protein | 8.10E−03 | 6014.06 | Q8IVM4 | 1 | 1 | 1 |
| 263 | A | FA20C_HUMAN (Q8IXL6) Protein FAM20C precursor | 5.31E−03 | 64405.57 | Q8IXL6 | 1 | 1 | 1 |
| 264 | E | Q8IY66 (Q8IY66) DNA ligase IV (Ligase IV, DNA, ATP-de [symbol] | 7.76E−03 | 103904 | Q8IY66 | 1 | 1 | 1 |
| 265 | D | GP115_HUMAN (Q8IZF3) Probable G-protein coupled rec [symbol] | 5.14E−04 | 83775.66 | Q8IZF3 | 1 | 1 | 1 |
| 266 | B | Q8IZQ1 (Q8IZQ1) ALFY | 6.15E−03 | 394992 | Q8IZQ1 | 1 | 1 | 1 |
| 267 | D | AHI1_HUMAN (Q8N157) Jouberin (Abelson helper integra [symbol] | 2.76E−03 | 137029 | Q8N157 | 1 | 1 | 1 |
| 268 | C | Q8N1K5 (Q8N1K5) Hypothetical protein FLJ40584 | 5.82E−03 | 73625.31 | Q8N1K5 | 1 | 1 | 1 |
| 269 | D | Q8N3I3 (Q8N3I3) Hypothetical protein DKFZp761P18121 | 3.18E−03 | 81550.7 | Q8N3I3 | 1 | 1 | 1 |
| 270 | A | Q8N543 (Q8N543) Hypothetical protein FLJ10826 | 1.90E−03 | 63206.21 | Q8N543 | 1 | 1 | 1 |
| 271 | E | Q8N780 (Q8N780) Hypothetical protein FLJ25943 | 8.78E−04 | 33347.2 | Q8N780 | 1 | 1 | 1 |
| 272 | C | Q8N8Z1 (Q8N8Z1) Hypothetical protein FLJ38687 | 1.10E−03 | 16623.52 | Q8N8Z1 | 1 | 1 | 1 |
| 273 | A | Q8N998 (Q8N998) Hypothetical protein FLJ38159 | 7.01E−03 | 43781.59 | Q8N998 | 1 | 1 | 1 |
| 274 | D | Q8N9S2 (Q8N9S2) Hypothetical protein FLJ36635 | 5.25E−03 | 28973.04 | Q8N9S2 | 1 | 1 | 1 |
| 275 | E | Q8NAJ6 (Q8NAJ6) Hypothetical protein FLJ35251 | 1.24E−03 | 81745.5 | Q8NAJ6 | 1 | 1 | 1 |
| 276 | A | Q8NCU4 (Q8NCU4) Hypothetical protein | 9.83E−03 | 110499 | Q8NCU4 | 1 | 1 | 1 |
| 277 | E | Q8NDA2 (Q8NDA2) Hypothetical protein DKFZp434P0216 [symbol] | 7.52E−04 | 143799.3 | Q8NDA2 | 1 | 1 | 1 |
| 278 | B | Q8NHQ3 (Q8NHQ3) RBBP8 protein | 7.19E−05 | 102594.6 | Q8NHQ3 | 1 | 1 | 1 |
| 279 | A | Q8TAN9 (Q8TAN9) Oxoglutarate dehydrogenase-like | 6.91E−03 | 114423.7 | Q8TAN9 | 1 | 1 | 1 |
| 280 | E | Q8TBB5 (Q8TBB5) Kelch domain containing 4 | 1.01E−03 | 57855.43 | Q8TBB5 | 1 | 1 | 1 |
| 281 | B | Q8TBE7 (Q8TBE7) TMEM22 protein | 8.33E−03 | 46419.88 | Q8TBE7 | 1 | 1 | 1 |
| 282 | A | Q8TBZ0 (Q8TBZ0) Hypothetical protein KM-HN-1 | 6.67E−04 | 96664.98 | Q8TBZ0 | 1 | 1 | 1 |
| 283 | B | Q8TC05 (Q8TC05) Mdm4, transformed 3T3 cell double mi [symbol] | 5.19E−03 | 80695.09 | Q8TC05 | 1 | 1 | 1 |
| 284 | B | CHD6_HUMAN (Q8TD26) Chromodomain-helicase-DNA- [symbol] | 6.52E−03 | 305218.9 | Q8TD26 | 1 | 1 | 1 |
| 285 | B | PAR3L_HUMAN (Q8TEW8) Amyotrophic lateral sclerosis [symbol] | 9.52E−03 | 132412.5 | Q8TEW8 | 1 | 1 | 1 |
| 286 | E | Q8WVY7 (Q8WVY7) Ubiquitin-like domain containing CTD [symbol] | 8.32E−04 | 36781.22 | Q8WVY7 | 1 | 1 | 1 |
| 287 | D | Q8WWA9 (Q8WWA9) Cytochrome P450, family 20, subfa [symbol] | 9.30E−06 | 52460.08 | Q8WWA9 | 1 | 1 | 1 |
| 288 | A | ANKR7_HUMAN (Q92527) Ankyrin repeat domain protein | 8.25E−03 | 22573.66 | Q92527 | 1 | 1 | 1 |
| 289 | E | Q92735 (Q92735) FMI protein | 4.05E−03 | 161492.6 | Q92735 | 1 | 1 | 1 |
| 290 | A | PCSK5_HUMAN (Q92824) Proprotein convertase subtilisi [symbol] | 2.02E−03 | 101708.3 | Q92824 | 1 | 1 | 1 |
| 291 | C | SNPC3_HUMAN (Q92966) snRNA-activating protein comp [symbol] | 9.07E−03 | 46722.65 | Q92966 | 1 | 1 | 1 |
| 292 | A | IRF7_HUMAN (Q92985) Interferon regulatory factor 7 (IRF [symbol] | 7.33E−04 | 54244.25 | Q92985 | 1 | 1 | 1 |
| 293 | A | Q969T7 (Q969T7) Hypothetical protein MGC20781 | 5.37E−03 | 33551.02 | Q969T7 | 1 | 1 | 1 |
| 294 | D | Q96AG4 (Q96AG4) Hypothetical protein PRO1855 | 9.06E−03 | 34908.9 | Q96AG4 | 1 | 1 | 1 |
| 295 | A | ISG20_HUMAN (Q96AZ6) Interferon-stimulated gene 20 k [symbol] | 9.06E−05 | 20350.62 | Q96AZ6 | 1 | 1 | 1 |
| 296 | E | DGC14_HUMAN (Q96DF8) DGCR14 protein (DiGeorge sy [symbol] | 5.80E−04 | 52535.89 | Q96DF8 | 1 | 1 | 1 |
| 297 | C | GMCL1_HUMAN (Q96IK5) Germ cell-less protein-like 1 | 7.74E−03 | 58647.47 | Q96IK5 | 1 | 1 | 1 |
| 298 | A | Q96LU7 (Q96LU7) Hypothetical protein FLJ25056 | 1.08E−03 | 31208.16 | Q96LU7 | 1 | 1 | 1 |
| 299 | A | KAD7_HUMAN (Q96M32) Putative adenylate kinase 7 (EC [symbol] | 9.64E−03 | 82620.65 | Q96M32 | 1 | 1 | 1 |
| 300 | E | IPO9_HUMAN (Q96P70) Importin-9 (Imp9) (Ran-binding p [symbol] | 7.94E−04 | 115757.8 | Q96P70 | 1 | 1 | 1 |
| 301 | A | DB118_HUMAN (Q96PH6) Beta-defensin 118 precursor ( [symbol] | 5.68E−03 | 13604.79 | Q96PH6 | 1 | 1 | 1 |
| 302 | D | VPS35_HUMAN (Q96QK1) Vacuolar protein sorting 35 (V [symbol] | 4.26E−03 | 91649.07 | Q96QK1 | 1 | 1 | 1 |
| 303 | E | MAGI1_HUMAN (Q96QZ7) Membrane associated guanyla [symbol] | 7.80E−03 | 164540.2 | Q96QZ7 | 1 | 1 | 1 |
| 304 | D | MCCA_HUMAN (Q96RQ3) Methylcrotonoyl-CoA carboxyla [symbol] | 3.52E−03 | 80382.04 | Q96RQ3 | 1 | 1 | 1 |
| 305 | C | MINT_HUMAN (Q96T58) Msx2-interacting protein (SPEN [symbol] | 2.49E−03 | 402004.3 | Q96T58 | 1 | 1 | 1 |
| 306 | E | NIBL_HUMAN (Q96TA1) Niban-like protein (Meg-3) | 6.21E−03 | 82631.11 | Q96TA1 | 1 | 1 | 1 |
| 307 | B | K1C12_HUMAN (Q99456) Keratin, type I cytoskeletal 12 ( [symbol] | 1.59E−03 | 53478.53 | Q99456 | 1 | 1 | 1 |
| 308 | C | SPS2_HUMAN (Q99611) Selenide, water dikinase 2 (EC 2 [symbol] | 2.61E−03 | 47227.85 | Q99611 | 1 | 1 | 1 |
| 309 | D | KIF2C_HUMAN (Q99661) Kinesin-like protein KIF2C (Mito [symbol] | 5.02E−03 | 81261.21 | Q99661 | 1 | 1 | 1 |
| 310 | E | CLP24_HUMAN (Q9BSN7) Claudin-like protein 24 | 2.48E−03 | 24523.55 | Q9BSN7 | 1 | 1 | 1 |
| 311 | E | DATF1_HUMAN (Q9BTC0) Death-associated transcription [symbol] | 7.61E−03 | 129070.5 | Q9BTC0 | 1 | 1 | 1 |
| 312 | E | CHST6_HUMAN (Q9GZX3) Carbohydrate sulfotransferase [symbol] | 5.32E−03 | 44071.2 | Q9GZX3 | 1 | 1 | 1 |
| 313 | C | TNKS2_HUMAN (Q9H2K2) Tankyrase 2 (EC 2.4.2.30) (TA [symbol] | 1.90E−05 | 126838.5 | Q9H2K2 | 1 | 1 | 1 |
| 314 | C | Q9H2M8 (Q9H2M8) DC28 | 8.59E−03 | 64005.01 | Q9H2M8 | 1 | 1 | 1 |
| 315 | E | SMRCD_HUMAN (Q9H4L7) SWI/SNF-related, matrix ass [symbol] | 7.44E−03 | 117299.9 | Q9H4L7 | 1 | 1 | 1 |
| 316 | E | SYP2L_HUMAN (Q9H987) Synaptopodin 2-like protein | 7.97E−03 | 102406.1 | Q9H987 | 1 | 1 | 1 |
| 317 | C | Q9H9B0 (Q9H9B0) Hypothetical protein FLJ12883 | 9.96E−03 | 91169.25 | Q9H9B0 | 1 | 1 | 1 |
| 318 | A | Q9H9B6 (Q9H9B6) Hypothetical protein FLJ12873 | 4.63E−03 | 71307.3 | Q9H9B6 | 1 | 1 | 1 |
| 319 | D | LPHN3_HUMAN (Q9HAR2) Latrophilin-3 precursor (Calci [symbol] | 9.90E−03 | 161709.3 | Q9HAR2 | 1 | 1 | 1 |
| 320 | A | MAGE1_HUMAN (Q9HCI5) Melanoma-associated antigen | 6.56E−03 | 103189.7 | Q9HCI5 | 1 | 1 | 1 |
| 321 | D | OLFL3_HUMAN (Q9NRN5) Olfactomedin-like protein 3 pr [symbol] | 1.86E−03 | 45981.32 | Q9NRN5 | 1 | 1 | 1 |
| 322 | D | TB22B_HUMAN (Q9NU19) TBC1 domain family member [symbol] | 3.31E−03 | 59044.32 | Q9NU19 | 1 | 1 | 1 |
| 323 | B | CT038_HUMAN (Q9NUV7) Protein C20orf38 | 7.71E−03 | 19679.8 | Q9NUV7 | 1 | 1 | 1 |
| 324 | A | Q9NXE3 (Q9NXE3) Hypothetical protein FLJ20298 | 8.80E−04 | 26243.39 | Q9NXE3 | 1 | 1 | 1 |
| 325 | E | THUM1_HUMAN (Q9NXG2) THUMP domain containing p [symbol] | 7.48E−03 | 39290.82 | Q9NXG2 | 1 | 1 | 1 |
| 326 | B | TE2IP_HUMAN (Q9NYB0) Telomeric repeat binding factor | 1.44E−03 | 44232.88 | Q9NYB0 | 1 | 1 | 1 |
| 327 | E | ZN226_HUMAN (Q9NYT6) Zinc finger protein 226 | 4.95E−03 | 91861.71 | Q9NYT6 | 1 | 1 | 1 |
| 328 | A | GSCR1_HUMAN (Q9NZM4) Glioma tumor suppressor car [symbol] | 6.54E−03 | 152898.4 | Q9NZM4 | 1 | 1 | 1 |
| 329 | B | HCN3_HUMAN (Q9P1Z3) Potassium/sodium hyperpolariz [symbol] | 5.36E−03 | 85977.23 | Q9P1Z3 | 1 | 1 | 1 |
| 330 | A | CEP72_HUMAN (Q9P209) Centrosomal protein of 72 kDa [symbol] | 1.96E−03 | 71673.01 | Q9P209 | 1 | 1 | 1 |
| 331 | A | CING_HUMAN (Q9P2M7) Cingulin | 5.06E−03 | 136303.7 | Q9P2M7 | 1 | 1 | 1 |
| 332 | A | COPG2_HUMAN (Q9UBF2) Coatomer gamma-2 subunit ( [symbol] | 6.47E−03 | 97559.69 | Q9UBF2 | 1 | 1 | 1 |
| 333 | B | OPT_HUMAN (Q9UBM4) Opticin precursor (Oculoglycan) | 4.68E−07 | 37237.39 | Q9UBM4 | 1 | 1 | 1 |
| 334 | D | SL9A2_HUMAN (Q9UBY0) Sodium/hydrogen exchanger 2 [symbol] | 6.23E−04 | 91461.34 | Q9UBY0 | 1 | 1 | 1 |
| 335 | E | Q9UJU1 (Q9UJU1) Cytovillin 2 (Fragment) | 8.20E−03 | 16251.62 | Q9UJU1 | 1 | 1 | 1 |
| 336 | C | STML2_HUMAN (Q9UJZ1) Stomatin-like protein 2 (SLP-2) [symbol] | 2.48E−03 | 38510.22 | Q9UJZ1 | 1 | 1 | 1 |
| 337 | C | MYH13_HUMAN (Q9UKX3) Myosin-13 (Myosin heavy cha [symbol] | 5.60E−03 | 223539.2 | Q9UKX3 | 1 | 1 | 1 |
| 338 | D | TTC7A_HUMAN (Q9ULT0) Tetratricopeptide repeat protei [symbol] | 6.23E−03 | 96123.27 | Q9ULT0 | 1 | 1 | 1 |
| 339 | E | ALK_HUMAN (Q9UM73) ALK tyrosine kinase receptor pre [symbol] | 9.57E−03 | 176304.4 | Q9UM73 | 1 | 1 | 1 |
| 340 | D | ICAM5_HUMAN (Q9UMF0) Intercellular adhesion molecul [symbol] | 2.21E−03 | 97270.1 | Q9UMF0 | 1 | 1 | 1 |
| 341 | E | PCDA7_HUMAN (Q9UN72) Protocadherin alpha 7 precurs [symbol] | 5.95E−03 | 100802.9 | Q9UN72 | 1 | 1 | 1 |
| 342 | C | SOX13_HUMAN (Q9UN79) SOX-13 protein (Type 1 diabe [symbol] | 7.41E−03 | 98718.72 | Q9UN79 | 1 | 1 | 1 |
| 343 | E | RHG26_HUMAN (Q9UNA1) Rho-GTPase-activating protei [symbol] | 9.52E−03 | 92176.67 | Q9UNA1 | 1 | 1 | 1 |
| 344 | A | TF2AY_HUMAN (Q9UNN4) TFIIA-alpha and beta-like fact [symbol] | 8.05E−03 | 52412.98 | Q9UNN4 | 1 | 1 | 1 |
| 345 | E | CNTN6_HUMAN (Q9UQ52) Contactin 6 precursor (Neural [symbol] | 5.55E−03 | 113885.1 | Q9UQ52 | 1 | 1 | 1 |
| 346 | A | NALDL_HUMAN (Q9UQQ1) N-acetylated-alpha-linked aci [symbol] | 1.21E−03 | 80574.27 | Q9UQQ1 | 1 | 1 | 1 |
| 347 | C | ADIP_HUMAN (Q9Y2D8) Afadin- and alpha-actinin-binding [symbol] | 5.45E−03 | 71191.48 | Q9Y2D8 | 1 | 1 | 1 |
| 348 | D | CG141_HUMAN (Q9Y3E0) UPF0198 protein CGI-141 | 5.94E−05 | 15415.4 | Q9Y3E0 | 1 | 1 | 1 |
| 349 | D | ZN337_HUMAN (Q9Y3M9) Zinc finger protein 337 | 4.48E−03 | 86819.26 | Q9Y3M9 | 1 | 1 | 1 |
| 350 | C | TLN1_HUMAN (Q9Y490) Talin-1 | 3.76E−03 | 269596.3 | Q9Y490 | 1 | 1 | 1 |
| 351 | C | PCDGB_HUMAN (Q9Y5H2) Protocadherin gamma A11 pr [symbol] | 9.51E−03 | 101480.6 | Q9Y5H2 | 1 | 1 | 1 |
| 352 | A | WIF1_HUMAN (Q9Y5W5) Wnt inhibitory factor 1 precurso [symbol] | 5.15E−03 | 41499.8 | Q9Y5W5 | 1 | 1 | 1 |
| 353 | B | S12A7_HUMAN (Q9Y666) Solute carrier family 12 membe [symbol] | 1.59E−04 | 119072.7 | Q9Y666 | 1 | 1 | 1 |
| 354 | D | SNCAP_HUMAN (Q9Y6H5) Synphilin-1 (Alpha-synuclein-i [symbol] | 5.97E−03 | 100319 | Q9Y6H5 | 1 | 1 | 1 |

TABLE 6
Proteins identified in 25 runs (5 patients with diagnosed Macular Hole)
SEQUEST filters: p prob < 0.01; Xcorr vs charge state: 1.5 for [M + H]1+, 2.0 for [M + 2H]2+, 2.5 for [M + 3H]3+; DCn > 0.1

| | | | | | | Peptide | MH | Tot IDS |
|---|---|---|---|---|---|---|---|---|
| | Patients | Reference | P (pro) | MW | Accession | (Hits) | Counts | 25 runs |
| 1 | HGFIL | Serotransferrin precursor | 4.22E−14 | 76999.66 | P02787 | 20 | 5 | 25 |
| 2 | FGILH | Serum albumin precursor | 1.00E−30 | 69321.63 | P02768 | 11 | 5 | 25 |
| 3 | FLGIH | Keratin, type II cytoskeletal 1 | 9.37E−12 | 65846.88 | P04264 | 7 | 5 | 24 |
| 4 | HILFG | Interphotoreceptor retinoid-binding protein precursor | 2.22E−16 | 135277.8 | P10745 | 5 | 5 | 24 |
| 5 | FLGHI | Hypothetical protein | 1.27E−07 | 20655.36 | Q569I7 | 3 | 5 | 24 |
| 6 | LFIGH | Clusterin precursor | 2.92E−08 | 52461.05 | P10909 | 1 | 5 | 24 |
| 7 | HLIFG | Transthyretin precursor | 1.00E−30 | 15877.05 | P02766 | 4 | 5 | 23 |
| 8 | FHGIL | Ig gamma-1 chain C region | 1.35E−12 | 36083.16 | P01857 | 7 | 5 | 19 |
| 9 | LFIHG | Keratin 10 | 1.11E−15 | 57212.99 | Q14664 | 1 | 5 | 17 |
| 10 | LGFHI | Keratin, type I cytoskeletal 10 | 8.41E−08 | 59482.76 | P13645 | 5 | 5 | 16 |
| 11 | HFGLI | Alpha-1-acid glycoprotein 1 precursor | 5.78E−12 | 23496.77 | P02763 | 2 | 5 | 15 |
| 12 | FGHLI | Apolipoprotein A-II precursor | 6.42E−11 | 11167.9 | P02652 | 1 | 5 | 15 |
| 13 | FGIH | Apolipoprotein A-I precursor | 3.44E−14 | 30758.94 | P02647 | 9 | 4 | 15 |
| 14 | FLIHG | Hemopexin precursor | 5.33E−12 | 51643.32 | P02790 | 5 | 5 | 14 |
| 15 | LGIH | PH domain containing protein | 3.94E−05 | 53317.19 | Q8TD55 | 1 | 4 | 14 |
| 16 | FHI | Hemoglobin delta subunit | 1.04E−12 | 15914.25 | P02042 | 7 | 3 | 14 |
| 17 | LHGIF | Pigment epithelium-derived factor precursor | 1.57E−08 | 46313.36 | P36955 | 3 | 5 | 12 |
| 18 | FILHG | Hypothetical protein FLJ42076 | 8.88E−05 | 21036.72 | Q6ZVU4 | 1 | 5 | 12 |
| 19 | FIGLH | Transcription factor NF-E4 | 1.59E−04 | 19006.73 | Q86UQ8 | 1 | 5 | 12 |
| 20 | HGLF | Keratin, type II cytoskeletal | 2.77E−09 | 65825.37 | P35908 | 1 | 4 | 12 |
| 21 | HLIG | Keratin, type II cytoskeletal 1b | 4.43E−06 | 61650.4 | Q7Z794 | 1 | 4 | 12 |
| 22 | FHGI | Alpha 2 globin variant | 1.89E−12 | 15270.94 | Q53F97 | 5 | 4 | 10 |
| 23 | GLFH | Keratin, type I cytoskeletal 9 | 2.61E−10 | 62091.76 | P35527 | 1 | 4 | 10 |
| 24 | IGLH | Hypothetical protein | 6.10E−04 | 9188.304 | Q6PHR9 | 1 | 4 | 10 |
| 25 | FLIH | Hypothetical protein DKFZp686I04196 | 3.51E−11 | 46031.66 | Q6N093 | 3 | 4 | 9 |
| 26 | GHIL | Hypothetical protein FLJ40259 | 3.36E−04 | 66086.88 | Q8N7W7 | 1 | 4 | 9 |
| 27 | HLGI | Nuclear receptor-interacting factor | 1.57E−03 | 52754.03 | Q8WY14 | 1 | 4 | 9 |
| 28 | FGH | Alpha-1-antitrypsin precursor | 2.51E−13 | 46707.09 | P01009 | 3 | 3 | 9 |
| 29 | ILH | Protein KIAA1683 | 2.65E−04 | 127611.8 | Q9H0B3 | 1 | 3 | 9 |
| 30 | FHGI | IGKC protein | 1.11E−15 | 25919.92 | Q502W4 | 3 | 4 | 8 |
| 31 | HFLI | Dickkopf-related protein 3 precursor | 4.25E−08 | 38266.07 | Q9UBP4 | 2 | 4 | 8 |
| 32 | GHL | Adenylyl cyclase type V | 1.51E−04 | 39312.25 | Q7RTV7 | 1 | 3 | 8 |
| 33 | HFIL | Tumor necrosis factor | 2.32E−03 | 20099.21 | Q5VWH1 | 1 | 4 | 7 |
| 34 | GIHL | HNRBF-2 | 2.48E−04 | 31787.43 | Q9H2I2 | 1 | 4 | 7 |
| 35 | FIH | Beta-globin gene from a thalassemia patient | 3.34E−11 | 18918.59 | Q14473 | 7 | 3 | 7 |
| 36 | HIG | Epsilon-sarcoglycan precursor | 4.19E−04 | 49722.25 | O43556 | 1 | 3 | 7 |
| 37 | FIG | Receptor-type tyrosine-protein phosphatase delta | 2.34E−03 | 214623.5 | P23468 | 1 | 3 | 7 |
| 38 | FHG | Vitamin D-binding protein | 2.26E−10 | 52902.04 | Q6GTG1 | 1 | 3 | 7 |
| 39 | HIL | Cytochrome P450, family 20, subfamily A, | 2.11E−06 | 52460.08 | Q8WWA9 | 1 | 3 | 7 |
| 40 | LHGI | Double-stranded RNA-binding protein | 1.64E−03 | 63227.92 | O95793 | 1 | 4 | 6 |
| 41 | HLIG | Hypothetical protein TMEM55A | 3.73E−05 | 28062.26 | Q8N4L2 | 1 | 4 | 6 |
| 42 | ILH | KIAA1200 protein | 1.07E−03 | 155607.6 | Q9ULM0 | 2 | 3 | 6 |
| 43 | GHF | Complement C4-A precursor | 3.96E−08 | 192649.5 | P0C0L4 | 1 | 3 | 6 |
| 44 | FG | Hypothetical protein DKFZp686J11235 | 4.12E−10 | 54424.95 | Q6MZW0 | 4 | 2 | 6 |
| 45 | FG | Complement C3 precursor | 4.12E−10 | 187045.3 | P01024 | 3 | 2 | 6 |
| 46 | FH | IGLC2 protein | 1.61E−13 | 24784.13 | Q567P1 | 3 | 2 | 6 |
| 47 | HF | RhoGTPase regulating protein variant | 5.25E−04 | 130153.4 | Q6RJU1 | 1 | 2 | 6 |
| 48 | ILFG | Tuberin (Tuberous sclerosis 2 protein) | 1.14E−04 | 200621.4 | P49815 | 1 | 4 | 5 |
| 49 | GHIL | Hypothetical protein FAM33A | 1.51E−03 | 14179.33 | Q8WVK7 | 1 | 4 | 5 |
| 50 | GLI | Vacuolar protein sorting 13A | 3.28E−03 | 360046.1 | Q96RL7 | 2 | 3 | 5 |
| 51 | LHF | Apolipoprotein E precursor | 3.48E−08 | 36131.79 | P02649 | 1 | 3 | 5 |
| 52 | LGH | Novel protein | 1.60E−03 | 107275.7 | Q5T7N3 | 1 | 3 | 5 |
| 53 | IGH | Hypothetical protein FLJ35435 | 1.80E−05 | 65308.66 | Q8NAF6 | 1 | 3 | 5 |
| 54 | GHL | Kallikrein-14 precursor | 5.06E−04 | 27434.89 | Q9P0G3 | 1 | 3 | 5 |
| 55 | FH | Apolipoprotein A-IV precursor | 4.69E−12 | 45343.52 | P06727 | 3 | 2 | 5 |
| 56 | HF | KIAA0425 protein | 2.12E−04 | 141056.5 | O43308 | 1 | 2 | 5 |
| 57 | LH | MYH7B protein | 5.17E−04 | 114574.5 | Q96I57 | 1 | 2 | 5 |
| 58 | F | Ceruloplasmin precursor | 3.13E−10 | 122127.6 | P00450 | 2 | 1 | 5 |
| 59 | L | Alpha-2-macroglobulin precursor | 5.36E−09 | 163174.3 | P01023 | 2 | 1 | 5 |
| 60 | F | VH1 protein precursor | 1.60E−07 | 17292.5 | O95978 | 1 | 1 | 5 |
| 61 | L | Osteopontin precursor | 5.20E−10 | 35401.25 | P10451 | 1 | 1 | 5 |
| 62 | GHL | Intercellular adhesion molecule 3 precursor | 2.21E−03 | 59345.85 | P32942 | 1 | 3 | 4 |
| 63 | FGL | PML-RARA regulated adaptor molecule | 1.05E−03 | 73950.18 | Q8N6W7 | 1 | 3 | 4 |
| 64 | HIG | Protein C19orf10 precursor (Stromal cell-derived GF) | 2.10E−03 | 18783.32 | Q969H8 | 1 | 3 | 4 |
| 65 | GIL | Calcium-independent phospholipase A2 | 4.48E−03 | 88421.11 | Q9NP80 | 1 | 3 | 4 |
| 66 | FG | Alpha-2-HS-glycoprotein precursor | 1.29E−06 | 39299.73 | P02765 | 2 | 2 | 4 |
| 67 | IL | Dystroglycan precursor | 1.67E−03 | 97519.91 | Q14118 | 2 | 2 | 4 |
| 68 | GI | Sortilin-related receptor precursor | 2.94E−04 | 248280 | Q92673 | 1 | 2 | 4 |
| 69 | G | Ig kappa chain V-II region | 4.45E−07 | 12668.32 | P01614 | 1 | 1 | 4 |
| 70 | G | Ig kappa chain V-II region MIL | 4.45E−07 | 12048.05 | P01616 | 1 | 1 | 4 |
| 71 | F | Ig gamma-3 chain C region | 2.03E−09 | 32309.81 | P01860 | 1 | 1 | 4 |
| 72 | F | Beta-2-glycoprotein I precursor | 3.39E−08 | 38272.67 | P02749 | 1 | 1 | 4 |
| 73 | F | Piccolo protein (Aczonin) | 4.86E−04 | 566313.1 | Q9Y6V0 | 1 | 1 | 4 |
| 74 | HLI | Zinc finger protein 197 | 4.48E−04 | 118770.8 | O14709 | 1 | 3 | 3 |
| 75 | FGH | Alpha-1B-glycoprotein precursor | 1.32E−09 | 54238.7 | P04217 | 1 | 3 | 3 |
| 76 | HLG | FES_HUMAN (P07332) Proto-oncogene tyrosine-protein k [symbol] | 1.22E−03 | 93412.31 | P07332 | 1 | 3 | 3 |
| 77 | GIF | Multidrug resistance-associated protein 1 | 3.76E−03 | 171450.5 | P33527 | 1 | 3 | 3 |
| 78 | FIG | Thrombospondin-2 precursor | 1.62E−03 | 129871.5 | P35442 | 1 | 3 | 3 |
| 79 | LIH | Neuronal membrane glycoprotein M6-a | 4.39E−03 | 31188.39 | P51674 | 1 | 3 | 3 |
| 80 | HLG | WD-repeat protein 9 | 1.15E−03 | 257060.4 | Q9NSI6 | 1 | 3 | 3 |
| 81 | HLI | ADAM 28 precursor | 2.13E−03 | 87151.09 | Q9UKQ2 | 1 | 3 | 3 |
| 82 | FL | Gelsolin precursor | 2.02E−04 | 85644.25 | P06396 | 1 | 2 | 3 |
| 83 | GH | Basement membrane-specific heparan sulfate proteoglyca [symbol] | 3.32E−03 | 468528.2 | P98160 | 1 | 2 | 3 |
| 84 | LH | Para-hydroxybenzoate-polyprenyltransferase | 3.67E−03 | 45578.79 | Q684R2 | 1 | 2 | 3 |
| 85 | FI | HSAL5836 | 2.94E−03 | 9682.161 | Q6UWG9 | 1 | 2 | 3 |
| 86 | IH | Hypothetical protein FLJ44832 | 2.50E−03 | 139335.1 | Q6ZQT3 | 1 | 2 | 3 |
| 87 | IH | Hypothetical protein FLJ40243 | 3.12E−03 | 180566.4 | Q7Z745 | 1 | 2 | 3 |
| 88 | IL | Hypothetical protein KIAA1505 | 2.15E−03 | 112734.9 | Q8IYE0 | 1 | 2 | 3 |
| 89 | LI | Keratin, type I cytoskeletal 12 | 7.68E−06 | 53478.53 | Q99456 | 1 | 2 | 3 |
| 90 | L | Nuclear pore complex protein Nup153 | 5.51E−03 | 153793.4 | P49790 | 1 | 1 | 3 |
| 91 | F | Keratin, type II cytoskeletal 2 | 4.77E−04 | 65830.09 | Q01546 | 1 | 1 | 3 |
| 92 | H | Secreted frizzled-related protein 3 precursor | 5.30E−06 | 36230.29 | Q92765 | 1 | 1 | 3 |
| 93 | GI | Zinc finger protein 185 | 4.64E−03 | 49156.87 | O15231 | 1 | 2 | 2 |
| 94 | IL | A-kinase anchor protein 3 | 2.47E−05 | 94676.41 | O75969 | 1 | 2 | 2 |
| 95 | HL | Alpha-1-antichymotrypsin precursor | 2.73E−03 | 47620.63 | P01011 | 1 | 2 | 2 |
| 96 | FG | Plasma retinol-binding protein precursor | 6.64E−03 | 22995.26 | P02753 | 1 | 2 | 2 |
| 97 | HG | Filamin-A | 1.64E−03 | 280583.4 | P21333 | 1 | 2 | 2 |
| 98 | HL | G protein-activated inward rectifier potassium channel | 1.22E−03 | 56566.8 | P48549 | 1 | 2 | 2 |
| 99 | GI | Telomeric repeat binding factor 1 | 2.43E−03 | 50313.11 | P54274 | 1 | 2 | 2 |
| 100 | HG | Trichohyalin | 3.52E−03 | 247074.4 | Q07283 | 1 | 2 | 2 |
| 101 | IH | Kinesin-like protein KIF1A | 3.48E−03 | 190963.1 | Q12756 | 1 | 2 | 2 |
| 102 | LH | Myosin-7A | 1.43E−03 | 254242.5 | Q13402 | 1 | 2 | 2 |
| 103 | GH | Ubiquitin conjugation factor E4 | 7.16E−05 | 122482 | Q14139 | 1 | 2 | 2 |
| 104 | IL | UDP-N-acetylhexosamine pyrophosphorylase | 2.88E−03 | 58731.98 | Q16222 | 1 | 2 | 2 |
| 105 | GH | Alpha-internexin | 6.89E−04 | 55357.48 | Q16352 | 1 | 2 | 2 |
| 106 | GF | Maguin-like protein variant II | 6.04E−03 | 61848.88 | Q5SGD5 | 1 | 2 | 2 |
| 107 | LH | RIF1_HUMAN (Q5UIP0) Telomere-associated protein RIF [symbol] | 2.56E−03 | 274293 | Q5UIP0 | 1 | 2 | 2 |
| 108 | GH | Q6P4H8 (Q6P4H8) Hypothetical protein LOC134145 | 3.97E−03 | 26123.47 | Q6P4H8 | 1 | 2 | 2 |
| 109 | GH | TRPM8_HUMAN (Q7Z2W7) Transient receptor potential c [symbol] | 7.58E−04 | 127573.3 | Q7Z2W7 | 1 | 2 | 2 |
| 110 | HL | Q8IV47 (Q8IV47) Fibromodulin, | 6.22E−04 | 43151.57 | Q8IV47 | 1 | 2 | 2 |
| 111 | LG | Q8N9V7 (Q8N9V7) Hypothetical protein FLJ36157 | 6.30E−04 | 77526.28 | Q8N9V7 | 1 | 2 | 2 |
| 112 | HG | LIPB2_HUMAN (Q8ND30) Liprin-beta 2 (Protein tyrosine p [symbol] | 3.34E−03 | 98383.37 | Q8ND30 | 1 | 2 | 2 |
| 113 | LG | Q8NDD1 (Q8NDD1) Hypothetical protein DKFZp547B1713 [symbol] | 5.52E−03 | 32732.49 | Q8NDD1 | 1 | 2 | 2 |
| 114 | GF | SYNE1_HUMAN (Q8NF91) Nesprin-1 (Nuclear envelope s [symbol] | 1.60E−03 | 1010433 | Q8NF91 | 1 | 2 | 2 |
| 115 | LH | Q8NHQ3 (Q8NHQ3) RBBP8 protein | 6.21E−05 | 102594.6 | Q8NHQ3 | 1 | 2 | 2 |
| 116 | LI | PCSK5_HUMAN (Q92824) Proprotein convertase subtilisir [symbol] | 2.56E−04 | 101708.3 | Q92824 | 1 | 2 | 2 |
| 117 | LH | K1849_HUMAN (Q96JH8) Protein KIAA1849 | 6.35E−03 | 116630 | Q96JH8 | 1 | 2 | 2 |
| 118 | HL | Q96S02 (Q96S02) Hypothetical protein gs80 | 3.06E−03 | 25483.99 | Q96S02 | 1 | 2 | 2 |
| 119 | LH | MINT_HUMAN (Q96T58) Msx2-interacting protein (SPEN [symbol] | 2.71E−03 | 402004.3 | Q96T58 | 1 | 2 | 2 |
| 120 | LH | GGA2_HUMAN (Q9UJY4) ADP-ribosylation factor binding | 3.44E−03 | 67133.75 | Q9UJY4 | 1 | 2 | 2 |
| 121 | HL | Q9ULH9 (Q9ULH9) KIAA1241 protein (Fragment) | 1.73E−05 | 97158.45 | Q9ULH9 | 1 | 2 | 2 |
| 122 | LG | Q9Y2W9 (Q9Y2W9) Endocrine regulator | 1.57E−04 | 231761.3 | Q9Y2W9 | 1 | 2 | 2 |
| 123 | LH | CT004_HUMAN (Q9Y312) Protein C20orf4 | 5.11E−03 | 43444.13 | Q9Y312 | 1 | 2 | 2 |
| 124 | GF | KIF3A_HUMAN (Q9Y496) Kinesin-like protein KIF3A (Micr [symbol] | 8.14E−04 | 80335.11 | Q9Y496 | 1 | 2 | 2 |
| 125 | I | CAC1G_HUMAN (O43497) Voltage-dependent T-type calc [symbol] | 2.51E−03 | 262301.1 | O43497 | 1 | 1 | 2 |
| 126 | I | GLU2B_HUMAN (P14314) Glucosidase II beta subunit pre [symbol] | 1.97E−03 | 59258.88 | P14314 | 1 | 1 | 2 |
| 127 | F | PTGDS_HUMAN (P41222) Prostaglandin-H2 D-isomerase [symbol] | 7.86E−08 | 21015.35 | P41222 | 1 | 1 | 2 |
| 128 | H | ATS12_HUMAN (P58397) ADAMTS-12 precursor (EC 3.4. [symbol] | 2.16E−03 | 177429.5 | P58397 | 1 | 1 | 2 |
| 129 | F | HBG1_HUMAN (P69891) Hemoglobin gamma-1 subunit ( [symbol] | 8.59E−03 | 15999.27 | P69891 | 1 | 1 | 2 |
| 130 | F | APXL_HUMAN (Q13796) Apical-like protein (APXL protein [symbol] | 1.55E−03 | 176302 | Q13796 | 1 | 1 | 2 |
| 131 | I | UBP10_HUMAN (Q14694) Ubiquitin carboxyl-terminal hyd [symbol] | 1.05E−03 | 87079.84 | Q14694 | 1 | 1 | 2 |
| 132 | I | Q15394 (Q15394) KIAA0005 protein (Fragment) | 6.56E−04 | 48615.16 | Q15394 | 1 | 1 | 2 |
| 133 | G | Q4V312 (Q4V312) Colony stimulating factor 2 receptor, alp [symbol] | 6.07E−04 | 46871.63 | Q4V312 | 1 | 1 | 2 |
| 134 | I | Q4V9L7 (Q4V9L7) NGRN protein (Fragment) | 4.36E−03 | 31332.13 | Q4V9L7 | 1 | 1 | 2 |
| 135 | I | Q53SW1 (Q53SW1) Hypothetical protein FLJ20254 | 2.53E−03 | 48539.34 | Q53SW1 | 1 | 1 | 2 |
| 136 | H | Q5BLQ2 (Q5BLQ2) Mucin | 5.22E−03 | 289658.2 | Q5BLQ2 | 1 | 1 | 2 |
| 137 | F | Q5JRC3 (Q5JRC3) RPEL repeat containing 1 (Fragment) | 4.33E−03 | 25543.2 | Q5JRC3 | 1 | 1 | 2 |
| 138 | F | Q5T2J8 (Q5T2J8) OTTHUMP00000018139 (Fragment) | 1.44E−03 | 97147.08 | Q5T2J8 | 1 | 1 | 2 |
| 139 | H | Q659E3 (Q659E3) Hypothetical protein DKFZp434I138 | 1.86E−03 | 78784.1 | Q659E3 | 1 | 1 | 2 |
| 140 | G | Q65ZC8 (Q65ZC8) Single-chain Fv (Fragment) | 3.05E−05 | 26110.55 | Q65ZC8 | 1 | 1 | 2 |
| 141 | F | Q6NUI2 (Q6NUI2) LOC150763 protein | 5.06E−04 | 87507.18 | Q6NUI2 | 1 | 1 | 2 |
| 142 | L | Q6PHR8 (Q6PHR8) Hypothetical protein (Fragment) | 2.10E−03 | 33452.8 | Q6PHR8 | 1 | 1 | 2 |
| 143 | G | Q6ZTG8 (Q6ZTG8) Hypothetical protein FLJ44670 | 3.09E−03 | 115755 | Q6ZTG8 | 1 | 1 | 2 |
| 144 | G | Q6ZV44 (Q6ZV44) Hypothetical protein FLJ43007 | 6.95E−04 | 19332.49 | Q6ZV44 | 1 | 1 | 2 |
| 145 | F | Q702P2 (Q702P2) VPS13C-2B protein | 4.75E−03 | 407940.5 | Q702P2 | 1 | 1 | 2 |
| 146 | I | VPS36_HUMAN (Q86VN1) Vacuolar protein sorting protei [symbol] | 1.09E−03 | 43788.79 | Q86VN1 | 1 | 1 | 2 |
| 147 | F | Q8N7Q3 (Q8N7Q3) Hypothetical protein FLJ40479 | 1.50E−03 | 45893.4 | Q8N7Q3 | 1 | 1 | 2 |
| 148 | I | RYR2_HUMAN (Q92736) Ryanodine receptor 2 (Cardiac [symbol] | 6.98E−03 | 564136.1 | Q92736 | 1 | 1 | 2 |
| 149 | I | TOP1M_HUMAN (Q969P6) DNA topoisomerase I, mitoch [symbol] | 5.03E−03 | 69828.34 | Q969P6 | 1 | 1 | 2 |
| 150 | F | Q96EK0 (Q96EK0) Hypothetical protein MGC4655 | 1.44E−03 | 41865.86 | Q96EK0 | 1 | 1 | 2 |
| 151 | I | GPTC3_HUMAN (Q96I76) G patch domain containing pro [symbol] | 4.90E−03 | 59301.11 | Q96I76 | 1 | 1 | 2 |
| 152 | F | Q9BRQ8 (Q9BRQ8) Apoptosis-inducing factor (AIF)-like m [symbol] | 2.48E−03 | 40501.28 | Q9BRQ8 | 1 | 1 | 2 |
| 153 | I | Q9BS34 (Q9BS34) Zinc finger protein 670 (Hypothetical pr [symbol] | 7.16E−03 | 44574.27 | Q9BS34 | 1 | 1 | 2 |
| 154 | I | PLEA5_HUMAN (Q9HAU0) Pleckstrin homology domain-c [symbol] | 3.50E−03 | 127384.5 | Q9HAU0 | 1 | 1 | 2 |
| 155 | G | Q9NXK0 (Q9NXK0) Hypothetical protein FLJ20202 | 1.52E−03 | 44899.86 | Q9NXK0 | 1 | 1 | 2 |
| 156 | F | COPG2_HUMAN (Q9UBF2) Coatomer gamma-2 subunit ( [symbol] | 1.54E−03 | 97559.69 | Q9UBF2 | 1 | 1 | 2 |
| 157 | I | RT02_HUMAN (Q9Y399) Mitochondrial 28S ribosomal pro [symbol] | 9.43E−05 | 33228.08 | Q9Y399 | 1 | 1 | 2 |
| 158 | H | Q5VXU2 (Q5VXU2) Propionyl Coenzyme A carboxylase, a [symbol] | 1.34E−04 | 80235.37 | Q5VXU2 | 2 | 1 | 1 |
| 159 | G | SGK1_HUMAN (O00141) Serine/threonine-protein kinase [symbol] | 1.08E−03 | 48925.35 | O00141 | 1 | 1 | 1 |
| 160 | I | TR10A_HUMAN (O00220) Tumor necrosis factor receptor | 3.21E−04 | 50028.81 | O00220 | 1 | 1 | 1 |
| 161 | F | O14913 (O14913) Kruppel-associated box protein | 2.39E−03 | 52045.66 | O14913 | 1 | 1 | 1 |
| 162 | G | HAT1_HUMAN (O14929) Histone acetyltransferase type B [symbol] | 1.43E−03 | 49480.84 | O14929 | 1 | 1 | 1 |
| 163 | F | O15014 (O15014) KIAA0295 protein (Fragment) | 2.79E−03 | 106382.5 | O15014 | 1 | 1 | 1 |
| 164 | L | PDZK3_HUMAN (O15018) PDZ domain containing protein [symbol] | 2.91E−03 | 301423.4 | O15018 | 1 | 1 | 1 |
| 165 | F | DMN_HUMAN (O15061) Desmuslin | 2.87E−03 | 172662.5 | O15061 | 1 | 1 | 1 |
| 166 | G | SOX12_HUMAN (O15370) SOX-12 protein (SOX-22 prote [symbol] | 1.92E−03 | 34279.67 | O15370 | 1 | 1 | 1 |
| 167 | F | O43719 (O43719) HIV TAT specific factor 1 | 2.48E−03 | 85800.38 | O43719 | 1 | 1 | 1 |
| 168 | F | KPRB_HUMAN (O60256) Phosphoribosyl pyrophosphate [symbol] | 3.63E−05 | 40899.39 | O60256 | 1 | 1 | 1 |
| 169 | H | LSD1_HUMAN (O60341) Lysine-specific histone demethyl [symbol] | 2.99E−04 | 92844.88 | O60341 | 1 | 1 | 1 |
| 170 | F | TBCD4_HUMAN (O60343) TBC1 domain family member [symbol] | 3.13E−03 | 146470.8 | O60343 | 1 | 1 | 1 |
| 171 | L | FZD6_HUMAN (O60353) Frizzled 6 precursor (Frizzled-6) | 1.13E−03 | 79222.06 | O60353 | 1 | 1 | 1 |
| 172 | I | TLR5_HUMAN (O60602) Toll-like receptor 5 precursor (To [symbol] | 5.88E−03 | 97663.38 | O60602 | 1 | 1 | 1 |
| 173 | H | O60826 (O60826) JM1 protein (Hypothetical protein CCD [symbol] | 2.74E−03 | 70712.32 | O60826 | 1 | 1 | 1 |
| 174 | G | GAS7_HUMAN (O60861) Growth-arrest-specific protein 7 | 3.84E−04 | 47236.75 | O60861 | 1 | 1 | 1 |
| 175 | L | O75179 (O75179) KIAA0697 protein (Fragment) | 1.34E−03 | 263079.2 | O75179 | 1 | 1 | 1 |
| 176 | F | PRPU_HUMAN (O94906) U5 snRNP-associated 102 kDa | 8.74E−03 | 106857.9 | O94906 | 1 | 1 | 1 |
| 177 | F | SLIK3_HUMAN (O94933) SLIT and NTRK-like protein 3 pr [symbol] | 9.29E−03 | 108937.1 | O94933 | 1 | 1 | 1 |
| 178 | L | O95285 (O95285) Erythroblast macrophage protein EMP | 8.16E−03 | 43879.31 | O95285 | 1 | 1 | 1 |
| 179 | L | ZIC2_HUMAN (O95409) Zinc finger protein ZIC 2 (Zinc fin [symbol] | 2.32E−03 | 54971.09 | O95409 | 1 | 1 | 1 |
| 180 | L | SVIL_HUMAN (O95425) Supervillin (Archvillin) (p205/p250 [symbol] | 6.30E−03 | 247550.6 | O95425 | 1 | 1 | 1 |
| 181 | F | FMNL_HUMAN (O95466) Formin-like 1 protein (Formin-lik [symbol] | 9.19E−03 | 121750.9 | O95466 | 1 | 1 | 1 |
| 182 | H | HPT_HUMAN (P00738) Haptoglobin precursor [Contains: I [symbol] | 2.71E−05 | 45176.59 | P00738 | 1 | 1 | 1 |
| 183 | H | CYTC_HUMAN (P01034) Cystatin C precursor (Neuroend [symbol] | 2.77E−05 | 15789.08 | P01034 | 1 | 1 | 1 |
| 184 | G | KV2B_HUMAN (P01615) Ig kappa chain V-II region FR | 5.81E−04 | 12651.75 | P01615 | 1 | 1 | 1 |
| 185 | I | CO3A1_HUMAN (P02461) Collagen alpha 1(III) chain prec [symbol] | 4.25E−03 | 138470.2 | P02461 | 1 | 1 | 1 |
| 186 | L | K2C6A_HUMAN (P02538) Keratin, type II cytoskeletal 6A ( [symbol] | 7.41E−07 | 59877.28 | P02538 | 1 | 1 | 1 |
| 187 | F | FIBG_HUMAN (P02679) Fibrinogen gamma chain precurs [symbol] | 1.61E−04 | 51478.88 | P02679 | 1 | 1 | 1 |
| 188 | H | CRP_HUMAN (P02741) C-reactive protein precursor [Con [symbol] | 9.82E−03 | 25022.68 | P02741 | 1 | 1 | 1 |
| 189 | I | AMBP_HUMAN (P02760) AMBP protein precursor [Contai [symbol] | 1.37E−04 | 38973.99 | P02760 | 1 | 1 | 1 |
| 190 | F | VTNC_HUMAN (P04004) Vitronectin precursor (Serum sp [symbol] | 5.97E−08 | 54271.23 | P04004 | 1 | 1 | 1 |
| 191 | F | CSF1R_HUMAN (P07333) Macrophage colony-stimulating [symbol] | 8.66E−03 | 107915.2 | P07333 | 1 | 1 | 1 |
| 192 | F | RET_HUMAN (P07949) Proto-oncogene tyrosine-protein k [symbol] | 4.41E−03 | 124239.4 | P07949 | 1 | 1 | 1 |
| 193 | L | RARA_HUMAN (P10276) Retinoic acid receptor alpha (RA [symbol] | 5.40E−04 | 50738.42 | P10276 | 1 | 1 | 1 |
| 194 | F | CP2D6_HUMAN (P10635) Cytochrome P450 2D6 (EC 1.1 [symbol] | 4.37E−03 | 55766.2 | P10635 | 1 | 1 | 1 |
| 195 | H | C1TC_HUMAN (P11586) C-1-tetrahydrofolate synthase, c [symbol] | 2.60E−03 | 101364.5 | P11586 | 1 | 1 | 1 |
| 196 | L | CSPG2_HUMAN (P13611) Versican core protein precurso [symbol] | 7.15E−06 | 372589 | P13611 | 1 | 1 | 1 |
| 197 | L | K1C13_HUMAN (P13646) Keratin, type I cytoskeletal 13 ( [symbol] | 4.22E−03 | 49555.45 | P13646 | 1 | 1 | 1 |
| 198 | H | PDE6A_HUMAN (P16499) Rod cGMP-specific 3′,5′-cyclic [symbol] | 9.31E−03 | 99438.76 | P16499 | 1 | 1 | 1 |
| 199 | F | A1AG2_HUMAN (P19652) Alpha-1-acid glycoprotein 2 pre [symbol] | 4.50E−06 | 23587.64 | P19652 | 1 | 1 | 1 |
| 200 | H | NEBU_HUMAN (P20929) Nebulin | 7.84E−04 | 772742.8 | P20929 | 1 | 1 | 1 |
| 201 | F | PRDX2_HUMAN (P32119) Peroxiredoxin 2 (EC 1.11.1.15) | 9.46E−03 | 21747.2 | P32119 | 1 | 1 | 1 |
| 202 | L | HYES_HUMAN (P34913) Epoxide hydrolase 2 (EC 3.3.2.3 [symbol] | 4.29E−04 | 62574.77 | P34913 | 1 | 1 | 1 |
| 203 | G | MYH11_HUMAN (P35749) Myosin-11 (Myosin heavy chair [symbol] | 2.94E−03 | 227197.9 | P35749 | 1 | 1 | 1 |
| 204 | G | BRCA1_HUMAN (P38398) Breast cancer type 1 susceptib [symbol] | 9.37E−03 | 207590.9 | P38398 | 1 | 1 | 1 |
| 205 | L | RL13A_HUMAN (P40429) 60S ribosomal protein L13a (23 [symbol] | 9.48E−03 | 23431.35 | P40429 | 1 | 1 | 1 |
| 206 | I | STAT1_HUMAN (P42224) Signal transducer and activator [symbol] | 7.68E−03 | 87279.63 | P42224 | 1 | 1 | 1 |
| 207 | H | RHG25_HUMAN (P42331) Rho-GTPase-activating protein [symbol] | 8.14E−03 | 72385.19 | P42331 | 1 | 1 | 1 |
| 208 | F | AFAM_HUMAN (P43652) Afamin precursor (Alpha-albumi [symbol] | 8.12E−05 | 69024.09 | P43652 | 1 | 1 | 1 |
| 209 | H | RBP2_HUMAN (P49792) Ran-binding protein 2 (RanBP2) | 3.16E−03 | 357991.4 | P49792 | 1 | 1 | 1 |
| 210 | I | TCPQ_HUMAN (P50990) T-complex protein 1, theta subu [symbol] | 6.50E−03 | 59451.57 | P50990 | 1 | 1 | 1 |
| 211 | F | DPOG1_HUMAN (P54098) DNA polymerase gamma subu [symbol] | 3.35E−03 | 139473.4 | P54098 | 1 | 1 | 1 |
| 212 | H | ELL_HUMAN (P55199) RNA polymerase II elongation fact [symbol] | 5.19E−03 | 68222.91 | P55199 | 1 | 1 | 1 |
| 213 | G | CAD12_HUMAN (P55289) Brain-cadherin precursor (BR-c [symbol] | 1.52E−03 | 88219.76 | P55289 | 1 | 1 | 1 |
| 214 | H | KR102_HUMAN (P60368) Keratin-associated protein 10-2 [symbol] | 1.72E−03 | 25595.75 | P60368 | 1 | 1 | 1 |
| 215 | H | PSME3_HUMAN (P61289) Proteasome activator complex | 5.60E−03 | 29487.55 | P61289 | 1 | 1 | 1 |
| 216 | L | RT21_HUMAN (P82921) Mitochondrial 28S ribosomal prot [symbol] | 2.34E−03 | 10734.48 | P82921 | 1 | 1 | 1 |
| 217 | I | NSBP1_HUMAN (P82970) Nucleosomal binding protein 1 | 1.93E−03 | 31505.95 | P82970 | 1 | 1 | 1 |
| 218 | G | SCN7A_HUMAN (Q01118) Sodium channel protein type V | 1.66E−03 | 193345 | Q01118 | 1 | 1 | 1 |
| 219 | I | BDH_HUMAN (Q02338) D-beta-hydroxybutyrate dehydrog [symbol] | 5.68E−03 | 38132.47 | Q02338 | 1 | 1 | 1 |
| 220 | L | CK013_HUMAN (Q02833) Protein C11orf13 (HRAS1-relat [symbol] | 3.17E−03 | 39920.6 | Q02833 | 1 | 1 | 1 |
| 221 | L | BPA1_HUMAN (Q03001) Bullous pemphigoid antigen 1 is [symbol] | 7.39E−03 | 371976.9 | Q03001 | 1 | 1 | 1 |
| 222 | F | ATP7A_HUMAN (Q04656) Copper-transporting ATPase 1 | 3.35E−03 | 163230 | Q04656 | 1 | 1 | 1 |
| 223 | L | APLP2_HUMAN (Q06481) Amyloid-like protein 2 precurso [symbol] | 1.03E−09 | 86900.28 | Q06481 | 1 | 1 | 1 |
| 224 | L | TRDN_HUMAN (Q13061) Triadin | 6.20E−03 | 81374.51 | Q13061 | 1 | 1 | 1 |
| 225 | F | CSN1_HUMAN (Q13098) COP9 signalosome complex su [symbol] | 7.31E−03 | 53338.1 | Q13098 | 1 | 1 | 1 |
| 226 | H | NOG2_HUMAN (Q13823) Nucleolar GTP-binding protein 2 [symbol] | 9.59E−03 | 83603.43 | Q13823 | 1 | 1 | 1 |
| 227 | H | Q14159 (Q14159) KIAA0146 protein (Fragment) | 1.44E−03 | 100592.3 | Q14159 | 1 | 1 | 1 |
| 228 | I | BMS1_HUMAN (Q14692) Ribosome biogenesis protein B [symbol] | 3.76E−03 | 145715.5 | Q14692 | 1 | 1 | 1 |
| 229 | L | 2A5D_HUMAN (Q14738) Serine/threonine protein phosph [symbol] | 2.98E−03 | 69947.5 | Q14738 | 1 | 1 | 1 |
| 230 | F | RBBP5_HUMAN (Q15291) Retinoblastoma-binding protei [symbol] | 9.99E−03 | 59044.69 | Q15291 | 1 | 1 | 1 |
| 231 | H | Q15299 (Q15299) RARB protein | 2.59E−04 | 11644 | Q15299 | 1 | 1 | 1 |
| 232 | F | CDC37_HUMAN (Q16543) Hso90 co-chaperone Cdc37 (H [symbol] | 8.92E−03 | 44440.05 | Q16543 | 1 | 1 | 1 |
| 233 | H | DUS5_HUMAN (Q16690) Dual specificity protein phospha [symbol] | 3.16E−04 | 42080.39 | Q16690 | 1 | 1 | 1 |
| 234 | H | Q49AA0 (Q49AA0) ZNF642 protein | 5.50E−03 | 61112.29 | Q49AA0 | 1 | 1 | 1 |
| 235 | G | Q4VC44 (Q4VC44) FLYWCH-type zinc finger 1, isoform a [symbol] | 1.11E−03 | 79985.74 | Q4VC44 | 1 | 1 | 1 |
| 236 | L | Q4VXM4 (Q4VXM4) Chromosome 20 open reading frame | 2.21E−03 | 114932.5 | Q4VXM4 | 1 | 1 | 1 |
| 237 | F | Q53EU6 (Q53EU6) Hypothetical protein (Fragment) | 9.80E−04 | 48673.52 | Q53EU6 | 1 | 1 | 1 |
| 238 | L | Q53RG2 (Q53RG2) Hypothetical protein CHRNG | 4.34E−03 | 58259.47 | Q53RG2 | 1 | 1 | 1 |
| 239 | L | Q562E5 (Q562E5) KIAA0194 protein (Fragment) | 3.73E−04 | 157553.1 | Q562E5 | 1 | 1 | 1 |
| 240 | G | Q59FI9 (Q59FI9) Ribosomal protein L12 variant (Fragmen [symbol] | 6.72E−03 | 21469.49 | Q59FI9 | 1 | 1 | 1 |
| 241 | H | Q5JPU1 (Q5JPU1) Pyruvate dehydrogenase (Lipoamide) [symbol] | 3.07E−03 | 22743.74 | Q5JPU1 | 1 | 1 | 1 |
| 242 | L | Q5JTH6 (Q5JTH6) Exosomal core protein CSL4 | 5.99E−04 | 18804.54 | Q5JTH6 | 1 | 1 | 1 |
| 243 | L | Q5JX69 (Q5JX69) OTTHUMP00000031352 | 8.08E−03 | 19485.96 | Q5JX69 | 1 | 1 | 1 |
| 244 | G | Q5T1J6 (Q5T1J6) OTTHUMP00000030508 | 4.35E−03 | 17161.46 | Q5T1J6 | 1 | 1 | 1 |
| 245 | H | Q5TDG2 (Q5TDG2) Hydroxy-delta-5-steroid dehydrogena [symbol] | 2.32E−03 | 42427.11 | Q5TDG2 | 1 | 1 | 1 |
| 246 | L | Q5TYW1 (Q5TYW1) Novel protein (Fragment) | 4.42E−03 | 97254.69 | Q5TYW1 | 1 | 1 | 1 |
| 247 | G | Q5VXI2 (Q5VXI2) Myosin IIIA | 7.99E−03 | 22191.69 | Q5VX12 | 1 | 1 | 1 |
| 248 | H | Q5VYM8 (Q5VYM8) Unc-13 homolog B (C. elegans) | 2.31E−03 | 180563.2 | Q5VYM8 | 1 | 1 | 1 |
| 249 | I | Q5XPV5 (Q5XPV5) Sjogren syndrome antigen A1 | 5.68E−04 | 54089.2 | Q5XPV5 | 1 | 1 | 1 |
| 250 | F | Q63HP7 (Q63HP7) Hypothetical protein DKFZp686P1551 | 5.39E−03 | 65621.97 | Q63HP7 | 1 | 1 | 1 |
| 251 | H | Q659F3 (Q659F3) Hypothetical protein DKFZp434J194 | 4.86E−03 | 14956.27 | Q659F3 | 1 | 1 | 1 |
| 252 | F | CEP4_HUMAN (Q66GS9) Centrosomal protein 4 (Centros [symbol] | 9.02E−04 | 133421.9 | Q66GS9 | 1 | 1 | 1 |
| 253 | I | Q68CI8 (Q68CI8) Hypothetical protein HMFT1272 (Fragm [symbol] | 2.38E−03 | 48598.88 | Q68CI8 | 1 | 1 | 1 |
| 254 | L | Q68DL8 (Q68DL8) Hypothetical protein DKFZp781L0319 | 3.98E−03 | 84645.59 | Q68DL8 | 1 | 1 | 1 |
| 255 | F | S6A19_HUMAN (Q695T7) Sodium-dependent neutral ami [symbol] | 5.44E−03 | 71063.29 | Q695T7 | 1 | 1 | 1 |
| 256 | L | Q6A334 (Q6A334) Mutated in bladder cancer 1 | 8.74E−03 | 61882.69 | Q6A334 | 1 | 1 | 1 |
| 257 | F | Q6IA31 (Q6IA31) FLJ14154 protein | 7.48E−03 | 27415.97 | Q6IA31 | 1 | 1 | 1 |
| 258 | G | Q6IBW4 (Q6IBW4) Em: U62317.2 protein | 6.70E−03 | 68183.7 | Q6IBW4 | 1 | 1 | 1 |
| 259 | I | Q6IE81 (Q6IE81) JADE1L protein | 9.07E−03 | 95472.82 | Q6IE81 | 1 | 1 | 1 |
| 260 | I | Q6IF05 (Q6IF05) Olfactory receptor OR1-47 | 8.84E−04 | 37737.82 | Q6IF05 | 1 | 1 | 1 |
| 261 | I | ST6B1_HUMAN (Q6IMI4) Sulfotransferase 6B1 (EC 2.8.2. [symbol] | 9.60E−03 | 30491.21 | Q6IMI4 | 1 | 1 | 1 |
| 262 | G | PTRF_HUMAN (Q6NZI2) Polymerase I and transcript rele [symbol] | 1.14E−03 | 43449.88 | Q6NZI2 | 1 | 1 | 1 |
| 263 | I | Q6P3W7 (Q6P3W7) Similar to mouse D10Ertd802e protei [symbol] | 9.06E−04 | 103642.4 | Q6P3W7 | 1 | 1 | 1 |
| 264 | F | Q6P658 (Q6P658) FNBP1 protein (Fragment) | 6.14E−04 | 40759.38 | Q6P658 | 1 | 1 | 1 |
| 265 | H | GP133_HUMAN (Q6QNK2) Probable G-protein coupled re [symbol] | 7.32E−03 | 96468.2 | Q6QNK2 | 1 | 1 | 1 |
| 266 | F | Q6UXX5 (Q6UXX5) ITI-like protein (Inter-alpha (Globulin) i [symbol] | 6.30E−03 | 143097.8 | Q6UXX5 | 1 | 1 | 1 |
| 267 | H | Q6ZMV7 (Q6ZMV7) Hypothetical protein FLJ16641 | 5.36E−03 | 45118.72 | Q6ZMV7 | 1 | 1 | 1 |
| 268 | L | Q6ZMX7 (Q6ZMX7) Hypothetical protein FLJ16607 | 1.23E−03 | 147809.7 | Q6ZMX7 | 1 | 1 | 1 |
| 269 | L | Q6ZQN2 (Q6ZQN2) Hypothetical protein FLJ46846 | 2.18E−03 | 180573.9 | Q6ZQN2 | 1 | 1 | 1 |
| 270 | H | Q6ZVL2 (Q6ZVL2) Hypothetical protein FLJ42427 | 7.92E−03 | 15702.14 | Q6ZVL2 | 1 | 1 | 1 |
| 271 | F | Q6ZW87 (Q6ZW87) Hypothetical protein FLJ41443 | 5.09E−03 | 24773.63 | Q6ZW87 | 1 | 1 | 1 |
| 272 | I | Q7KYS7 (Q7KYS7) Hypothetical protein (Fragment) | 1.53E−03 | 31128.16 | Q7KYS7 | 1 | 1 | 1 |
| 273 | F | Q7KZ97 (Q7KZ97) Antithrombin III variant | 3.10E−06 | 52657.96 | Q7KZ97 | 1 | 1 | 1 |
| 274 | G | Q7RTS7 (Q7RTS7) Keratin 5c | 4.83E−03 | 59333.45 | Q7RTS7 | 1 | 1 | 1 |
| 275 | F | CA036_HUMAN (Q7Z3Z2) Protein C1orf36 | 1.27E−04 | 22689.57 | Q7Z3Z2 | 1 | 1 | 1 |
| 276 | H | Q7Z402 (Q7Z402) Transmembrane channel-like protein 7 | 6.94E−03 | 83447.49 | Q7Z402 | 1 | 1 | 1 |
| 277 | I | Q7Z736 (Q7Z736) Hypothetical protein FLJ21019 | 7.52E−03 | 85273.38 | Q7Z736 | 1 | 1 | 1 |
| 278 | I | Q86VJ1 (Q86VJ1) E3 ligase for inhibin receptor | 9.51E−03 | 289427.4 | Q86VJ1 | 1 | 1 | 1 |
| 279 | G | Q86WI3 (Q86WI3) NOD27 | 2.47E−03 | 204535 | Q86WI3 | 1 | 1 | 1 |
| 280 | L | Q86Y22 (Q86Y22) Alpha 1 type XXIII collagen | 3.71E−03 | 51912.29 | Q86Y22 | 1 | 1 | 1 |
| 281 | F | Q8IUA7 (Q8IUA7) ATP-binding cassette sub-family A me [symbol] | 5.35E−03 | 184241.6 | Q8IUA7 | 1 | 1 | 1 |
| 282 | G | Q8IUG8 (Q8IUG8) Carnitine transporter 2 | 6.69E−03 | 60830.3 | Q8IUG8 | 1 | 1 | 1 |
| 283 | F | Q8IUS2 (Q8IUS2) LOC339483 protein | 3.48E−03 | 2870.257 | Q8IUS2 | 1 | 1 | 1 |
| 284 | I | Q8IXT9 (Q8IXT9) RUN domain containing 1 | 5.03E−03 | 67603.02 | Q8IXT9 | 1 | 1 | 1 |
| 285 | L | Q8IZA4 (Q8IZA4) ELYS transcription factor-like protein T [symbol] | 5.66E−03 | 256022.6 | Q8IZA4 | 1 | 1 | 1 |
| 286 | L | ASPM_HUMAN (Q8IZT6) Abnormal spindle-like microcep [symbol] | 4.72E−03 | 409539.8 | Q8IZT6 | 1 | 1 | 1 |
| 287 | G | Q8N1G4 (Q8N1G4) Hypothetical protein LRRC47 (Novel p [symbol] | 2.19E−03 | 63433.89 | Q8N1G4 | 1 | 1 | 1 |
| 288 | H | Q8N401 (Q8N401) KIAA1632 protein | 8.67E−03 | 52340.19 | Q8N401 | 1 | 1 | 1 |
| 289 | L | DCAK2_HUMAN (Q8N568) Serine/threonine-protein kinas [symbol] | 4.26E−03 | 83587.57 | Q8N568 | 1 | 1 | 1 |
| 290 | L | Q8N5E0 (Q8N5E0) Apoptosis-inducing factor like, isoform | 5.12E−03 | 65924.3 | Q8N5E0 | 1 | 1 | 1 |
| 291 | I | Q8N780 (Q8N780) Hypothetical protein FLJ25943 | 1.83E−03 | 33347.2 | Q8N780 | 1 | 1 | 1 |
| 292 | H | Q8N8Z1 (Q8N8Z1) Hypothetical protein FLJ38687 | 9.20E−03 | 16623.52 | Q8N8Z1 | 1 | 1 | 1 |
| 293 | F | Q8NC99 (Q8NC99) Hypothetical protein FLJ90396 | 5.35E−03 | 66758.55 | Q8NC99 | 1 | 1 | 1 |
| 294 | H | Q8NF06 (Q8NF06) FLJ00398 protein (Fragment) | 1.78E−04 | 72000.41 | Q8NF06 | 1 | 1 | 1 |
| 295 | G | Q8NFY8 (Q8NFY8) Neuroblastoma-amplified protein | 1.13E−03 | 268295.4 | Q8NFY8 | 1 | 1 | 1 |
| 296 | L | O10A7_HUMAN (Q8NGE5) Olfactory receptor 10A7 | 8.56E−03 | 35670.34 | Q8NGE5 | 1 | 1 | 1 |
| 297 | F | Q8NH29 (Q8NH29) Seven transmembrane helix receptor | 1.70E−04 | 25373.85 | Q8NH29 | 1 | 1 | 1 |
| 298 | G | IL17D_HUMAN (Q8TAD2) Interleukin-17D precursor (IL-1 [symbol] | 6.37E−03 | 21879.14 | Q8TAD2 | 1 | 1 | 1 |
| 299 | H | Q8TBE7 (Q8TBE7) TMEM22 protein | 7.86E−03 | 46419.88 | Q8TBE7 | 1 | 1 | 1 |
| 300 | F | Q8TCE1 (Q8TCE1) SERPINC1 protein | 2.30E−05 | 29073.79 | Q8TCE1 | 1 | 1 | 1 |
| 301 | F | Q8TCJ2 (Q8TCJ2) Source of immunodominant MHC-asso [symbol] | 3.14E−04 | 93613.76 | Q8TCJ2 | 1 | 1 | 1 |
| 302 | F | DDX54_HUMAN (Q8TDD1) DEAD-box protein 54 (EC 3.6. [symbol] | 1.74E−03 | 98534.24 | Q8TDD1 | 1 | 1 | 1 |
| 303 | F | K1199_HUMAN (Q8WUJ3) Protein KIAA1199 precursor | 9.51E−03 | 152900 | Q8WUJ3 | 1 | 1 | 1 |
| 304 | F | GCN1L_HUMAN (Q92616) GCN1-like protein 1 (HsGCN1) [symbol] | 7.47E−03 | 292424.8 | Q92616 | 1 | 1 | 1 |
| 305 | G | Q92772 (Q92772) P56 KKIAMRE protein kinase (Cyclin-d [symbol] | 6.77E−03 | 55982.93 | Q92772 | 1 | 1 | 1 |
| 306 | L | COR2A_HUMAN (Q92828) Coronin-2A (WD repeat-contai [symbol] | 3.04E−03 | 59725.43 | Q92828 | 1 | 1 | 1 |
| 307 | G | Q969T7 (Q969T7) Hypothetical protein MGC20781 | 6.70E−03 | 33551.02 | Q969T7 | 1 | 1 | 1 |
| 308 | G | DGC14_HUMAN (Q96DF8) DGCR14 protein (DiGeorge s [symbol] | 7.51E−03 | 52535.89 | Q96DF8 | 1 | 1 | 1 |
| 309 | G | Q96E20 (Q96E20) CD99L2 protein (Hypothetical protein) ( [symbol] | 3.63E−03 | 16612.17 | Q96E20 | 1 | 1 | 1 |
| 310 | L | Q96IV6 (Q96IV6) C5orf4 protein | 4.48E−03 | 38976.07 | Q96IV6 | 1 | 1 | 1 |
| 311 | L | RSRC1_HUMAN (Q96IZ7) Arginine/serine-rich coiled coil [symbol] | 8.17E−03 | 38654.46 | Q96IZ7 | 1 | 1 | 1 |
| 312 | G | KCNH8_HUMAN (Q96L42) Potassium voltage-gated chan [symbol] | 3.90E−04 | 123754.3 | Q96L42 | 1 | 1 | 1 |
| 313 | H | Q96LU7 (Q96LU7) Hypothetical protein FLJ25056 | 7.45E−03 | 31208.16 | Q96LU7 | 1 | 1 | 1 |
| 314 | F | Q96MP4 (Q96MP4) Hypothetical protein FLJ32091 | 6.67E−03 | 81413.94 | Q96MP4 | 1 | 1 | 1 |
| 315 | H | Q96NI8 (Q96NI8) Hypothetical protein FLJ30791 | 4.72E−03 | 62289.69 | Q96NI8 | 1 | 1 | 1 |
| 316 | H | ZN285_HUMAN (Q96NJ3) Zinc finger protein 285 | 2.66E−03 | 50009.57 | Q96NJ3 | 1 | 1 | 1 |
| 317 | G | IPO9_HUMAN (Q96P70) Importin-9 (Imp9) (Ran-binding p [symbol] | 9.18E−03 | 115757.8 | Q96P70 | 1 | 1 | 1 |
| 318 | H | INP4A_HUMAN (Q96PE3) Type I inositol-3,4-bisphosphat [symbol] | 2.98E−04 | 109885.5 | Q96PE3 | 1 | 1 | 1 |
| 319 | G | UHRF2_HUMAN (Q96PU4) Ubiquitin-like containing PHD [symbol] | 6.47E−03 | 89927.86 | Q96PU4 | 1 | 1 | 1 |
| 320 | G | Q96QE0 (Q96QE0) Hypothetical protein DL8Q12 | 3.39E−03 | 23959.2 | Q96QE0 | 1 | 1 | 1 |
| 321 | H | PANX2_HUMAN (Q96RD6) Pannexin-2 | 7.29E−03 | 69434.98 | Q96RD6 | 1 | 1 | 1 |
| 322 | G | Q96RF2 (Q96RF2) WWOXdelta5-8 | 3.55E−03 | 35019.5 | Q96RF2 | 1 | 1 | 1 |
| 323 | L | LYST_HUMAN (Q99698) Lysosomal trafficking regulator ( [symbol] | 7.47E−03 | 428865.6 | Q99698 | 1 | 1 | 1 |
| 324 | H | SMO_HUMAN (Q99835) Smoothened homolog precursor | 7.04E−05 | 86341.18 | Q99835 | 1 | 1 | 1 |
| 325 | L | Q99993 (Q99993) WUGSC:H_2G3A.1 protein | 4.36E−03 | 60051.64 | Q99993 | 1 | 1 | 1 |
| 326 | L | OSR10_HUMAN (Q9BXB5) Oxysterol binding protein-relat [symbol] | 7.10E−03 | 83917.38 | Q9BXB5 | 1 | 1 | 1 |
| 327 | H | EMR3_HUMAN (Q9BY15) EGF-like module containing mu [symbol] | 6.97E−03 | 72545.69 | Q9BY15 | 1 | 1 | 1 |
| 328 | F | Q9BYJ0 (Q9BYJ0) Ksp37 (HBp17-related protein) | 6.42E−04 | 24565.15 | Q9BYJ0 | 1 | 1 | 1 |
| 329 | G | OSR7_HUMAN (Q9BZF2) Oxysterol binding protein-relate [symbol] | 3.33E−04 | 95371.7 | Q9BZF2 | 1 | 1 | 1 |
| 330 | L | Q9BZH2 (Q9BZH2) False p73 target protein | 2.66E−03 | 53043.29 | Q9BZH2 | 1 | 1 | 1 |
| 331 | I | PHAR1_HUMAN (Q9C0D0) Phosphatase and actin regula [symbol] | 2.72E−04 | 54573.55 | Q9C0D0 | 1 | 1 | 1 |
| 332 | G | Q9C0D4 (Q9C0D4) KIAA1729 protein (Fragment) | 1.84E−03 | 113646.6 | Q9C0D4 | 1 | 1 | 1 |
| 333 | L | DDX24_HUMAN (Q9GZR7) ATP-dependent RNA helicase | 7.26E−03 | 96271.52 | Q9GZR7 | 1 | 1 | 1 |
| 334 | L | Q9GZT6 (Q9GZT6) MDS011 (Hypothetical protein MDS02 | 4.53E−03 | 29453.17 | Q9GZT6 | 1 | 1 | 1 |
| 335 | H | Q9H0J7 (Q9H0J7) Hypothetical protein DKFZp434F0116 | 2.81E−04 | 53323.07 | Q9H0J7 | 1 | 1 | 1 |
| 336 | L | GHITM_HUMAN (Q9H3K2) Growth hormone inducible trar [symbol] | 4.28E−03 | 37180.59 | Q9H3K2 | 1 | 1 | 1 |
| 337 | H | Q9H3M9 (Q9H3M9) Homo sapiens (Fragment) | 5.36E−03 | 39581.41 | Q9H3M9 | 1 | 1 | 1 |
| 338 | F | Q9H6E6 (Q9H6E6) Hypothetical protein FLJ22346 | 1.41E−03 | 81825.66 | Q9H6E6 | 1 | 1 | 1 |
| 339 | H | RANB3_HUMAN (Q9H6Z4) Ran-binding protein 3 (RanBP [symbol] | 3.82E−03 | 60173.32 | Q9H6Z4 | 1 | 1 | 1 |
| 340 | H | SMYD3_HUMAN (Q9H7B4) SET and MYND domain-cont [symbol] | 5.56E−03 | 49050.59 | Q9H7B4 | 1 | 1 | 1 |
| 341 | L | Q9H7C4 (Q9H7C4) Hypothetical protein FLJ21054 | 8.68E−03 | 17715.02 | Q9H7C4 | 1 | 1 | 1 |
| 342 | G | SYP2L_HUMAN (Q9H987) Synaptopodin 2-like protein | 8.46E−03 | 102406.1 | Q9H987 | 1 | 1 | 1 |
| 343 | L | COG4_HUMAN (Q9H9E3) Conserved oligomeric Golgi co [symbol] | 9.55E−03 | 89038.48 | Q9H9E3 | 1 | 1 | 1 |
| 344 | G | UBP29_HUMAN (Q9HBJ7) Ubiquitin carboxyl-terminal hyd [symbol] | 1.22E−03 | 104090.1 | Q9HBJ7 | 1 | 1 | 1 |
| 345 | H | Q9HBY0 (Q9HBY0) Putative superoxide-generating NADP [symbol] | 4.96E−03 | 64893.13 | Q9HBY0 | 1 | 1 | 1 |
| 346 | F | SYTL2_HUMAN (Q9HCH5) Synaptotagmin-like protein 2 ( [symbol] | 8.61E−04 | 100734.4 | Q9HCH5 | 1 | 1 | 1 |
| 347 | F | Q9NR16 (Q9NR16) Scavenger receptor cysteine-rich type | 6.17E−03 | 159159.5 | Q9NR16 | 1 | 1 | 1 |
| 348 | L | SPTN5_HUMAN (Q9NRC6) Spectrin beta chain, brain 4 ( [symbol] | 3.96E−04 | 416577.2 | Q9NRC6 | 1 | 1 | 1 |
| 349 | L | DC13_HUMAN (Q9NRP2) UPF0287 protein DC13 | 2.81E−03 | 9453.72 | Q9NRP2 | 1 | 1 | 1 |
| 350 | F | Q9NS87 (Q9NS87) Kinesin-like protein 2 | 4.45E−03 | 160060.4 | Q9NS87 | 1 | 1 | 1 |
| 351 | G | Q9NSN6 (Q9NSN6) Hypothetical protein DKFZp761O011 [symbol] | 6.56E−03 | 39970.1 | Q9NSN6 | 1 | 1 | 1 |
| 352 | H | EMIL3_HUMAN (Q9NT22) EMILIN-3 precursor (EMILIN-5) [symbol] | 3.69E−03 | 82595.73 | Q9NT22 | 1 | 1 | 1 |
| 353 | H | UGGG1_HUMAN (Q9NYU2) UDP-glucose:glycoprotein gl [symbol] | 3.83E−03 | 174866.5 | Q9NYU2 | 1 | 1 | 1 |
| 354 | H | T2R14_HUMAN (Q9NYV8) Taste receptor type 2 member | 1.33E−03 | 36136.48 | Q9NYV8 | 1 | 1 | 1 |
| 355 | F | COMD9_HUMAN (Q9P000) COMM domain containing pro [symbol] | 4.29E−04 | 21904.53 | Q9P000 | 1 | 1 | 1 |
| 356 | L | VPS18_HUMAN (Q9P253) Vacuolar protein sorting 18 (hV [symbol] | 7.01E−03 | 110115.9 | Q9P253 | 1 | 1 | 1 |
| 357 | I | RRBP1_HUMAN (Q9P2E9) Ribosome-binding protein 1 ( [symbol] | 4.24E−03 | 152380 | Q9P2E9 | 1 | 1 | 1 |
| 358 | G | SUCB1_HUMAN (Q9P2R7) Succinyl-CoA ligase [ADP-forr [symbol] | 8.47E−03 | 50299.33 | Q9P2R7 | 1 | 1 | 1 |
| 359 | L | OPT_HUMAN (Q9UBM4) Opticin precursor (Oculoglycan) | 1.45E−03 | 37237.39 | Q9UBM4 | 1 | 1 | 1 |
| 360 | I | GT2D1_HUMAN (Q9UHL9) General transcription factor II- [symbol] | 4.56E−03 | 105990.8 | Q9UHL9 | 1 | 1 | 1 |
| 361 | G | Q9UI74 (Q9UI74) PRO0245 | 7.12E−04 | 8075.004 | Q9UI74 | 1 | 1 | 1 |
| 362 | L | SPB13_HUMAN (Q9UIV8) Serpin B13 (Hurpin) (HaCaT U [symbol] | 8.72E−04 | 44248.2 | Q9UIV8 | 1 | 1 | 1 |
| 363 | I | HOOK1_HUMAN (Q9UJC3) Hook homolog 1 (h-hook1) (h [symbol] | 9.77E−03 | 84594.84 | Q9UJC3 | 1 | 1 | 1 |
| 364 | F | DKKL1_HUMAN (Q9UK85) Dickkopf-like protein 1 precurs [symbol] | 1.36E−04 | 26990.23 | Q9UK85 | 1 | 1 | 1 |
| 365 | H | Q9ULI2 (Q9ULI2) KIAA1238 protein (Fragment) | 2.40E−03 | 45141.04 | Q9ULI2 | 1 | 1 | 1 |
| 366 | L | PADI3_HUMAN (Q9ULW8) Protein-arginine deiminase typ [symbol] | 9.91E−04 | 74695.37 | Q9ULW8 | 1 | 1 | 1 |
| 367 | H | MLL4_HUMAN (Q9UMN6) Myeloid/lymphoid or mixed-line [symbol] | 9.43E−03 | 293327.3 | Q9UMN6 | 1 | 1 | 1 |
| 368 | I | PCDA7_HUMAN (Q9UN72) Protocadherin alpha 7 precurs [symbol] | 3.78E−03 | 100802.9 | Q9UN72 | 1 | 1 | 1 |
| 369 | F | RHG26_HUMAN (Q9UNA1) Rho-GTPase-activating protei [symbol] | 4.55E−03 | 92176.67 | Q9UNA1 | 1 | 1 | 1 |
| 370 | H | TF2AY_HUMAN (Q9UNN4) TFIIA-alpha and beta-like fact [symbol] | 4.50E−03 | 52412.98 | Q9UNN4 | 1 | 1 | 1 |
| 371 | H | GPR34_HUMAN (Q9UPC5) Probable G-protein coupled re [symbol] | 8.71E−03 | 43831.09 | Q9UPC5 | 1 | 1 | 1 |
| 372 | G | Q9UQ10 (Q9UQ10) Dimeric dihydrodiol dehydrogenase (E [symbol] | 9.73E−03 | 36358.77 | Q9UQ10 | 1 | 1 | 1 |
| 373 | I | SHOC2_HUMAN (Q9UQ13) Leucine-rich repeat protein S [symbol] | 3.26E−03 | 64847.64 | Q9UQ13 | 1 | 1 | 1 |
| 374 | F | ADIP_HUMAN (Q9Y2D8) Afadin- and alpha-actinin-binding [symbol] | 2.87E−03 | 71191.48 | Q9Y2D8 | 1 | 1 | 1 |
| 375 | G | AT11B_HUMAN (Q9Y2G3) Probable phospholipid-transpo [symbol] | 9.84E−03 | 134104.1 | Q9Y2G3 | 1 | 1 | 1 |
| 376 | F | PCDG7_HUMAN (Q9Y5G6) Protocadherin gamma A7 pre [symbol] | 2.96E−03 | 101659.2 | Q9Y5G6 | 1 | 1 | 1 |
| 377 | L | PCDA2_HUMAN (Q9Y5H9) Protocadherin alpha 2 precurs [symbol] | 6.26E−03 | 102000.4 | Q9Y5H9 | 1 | 1 | 1 |
| 378 | G | Q9Y5S5 (Q9Y5S5) DNA polymerase epsilon catalytic subu [symbol] | 9.10E−03 | 262846.1 | Q9Y5S5 | 1 | 1 | 1 |
| 379 | F | SO1B1_HUMAN (Q9Y6L6) Solute carrier organic anion tra [symbol] | 1.51E−03 | 76398.98 | Q9Y6L6 | 1 | 1 | 1 |

TABLE 7
Proteins identified uniquely in Retinal Detachment diagnosed patients (5 patients, 25 runs)
SEQUEST filters: p prob < 0.01; Xcorr vs charge state: 1.5 for [M + H]1+, 2.0 for [M + 2H]2+, 2.5 for [M + 3H]3+; DCn > 0.1

| | | | | | | | RD | |
|---|---|---|---|---|---|---|---|---|
| | Patients | Reference | P (pro) | MW | Accession | (Hits) | Counts | TOT ID (25) |
| 1 | CBDE | LOC131076 protein | 2.50E−03 | 16609.49 | Q4VC31 | 1 | 4 | 5 |
| 2 | EBC | Keratin, type II | 5.27E−07 | 54935.82 | Q14533 | 1 | 3 | 5 |
| 3 | CEA | Histone acetyltransferase | 6.68E−04 | 92953.77 | Q92831 | 1 | 3 | 4 |
| 4 | AB | Cathepsin D precursor | 3.66E−09 | 44523.66 | P07339 | 1 | 2 | 4 |
| 5 | BD | Peroxiredoxin 1 | 1.57E−06 | 22096.28 | Q06830 | 1 | 2 | 4 |
| 6 | ECD | Nuclear pore complex protein Nup214 | 4.38E−03 | 213632.8 | P35658 | 1 | 3 | 3 |
| 7 | DEA | T-cell activation Rho GTPase-activating protein | 5.60E−03 | 80652.9 | Q8N103 | 1 | 3 | 3 |
| 8 | EDC | Protein C9orf126 | 1.82E−03 | 70385.07 | Q8N9R8 | 1 | 3 | 3 |
| 9 | BE | Receptor interacting protein kinase 5 | 2.01E−03 | 99957.35 | Q5RKT0 | 1 | 2 | 3 |
| 10 | BD | Novel protein | 2.74E−04 | 91277.53 | Q5VVM6 | 1 | 2 | 3 |
| 11 | E | Potassium voltage-gated channel subfamily A | 8.04E−04 | 56505.6 | Q09470 | 1 | 1 | 3 |
| 12 | D | Huntingtin interacting protein E | 7.33E−05 | 51745.16 | Q9BVA6 | 1 | 1 | 3 |
| 13 | BE | KIAA0068 protein (Fragment) | 2.65E−04 | 147040.6 | Q14467 | 2 | 2 | 2 |
| 14 | CE | Hypothetical protein FLJ21129 | 2.87E−03 | 63276.44 | Q9H796 | 2 | 2 | 2 |
| 15 | CE | HS1-associating protein X-1 | 2.63E−03 | 31601.06 | O00165 | 1 | 2 | 2 |
| 16 | EA | Inositol polyphosphate 5-phosphatase | 1.69E−04 | 138498.9 | O15357 | 1 | 2 | 2 |
| 17 | DE | DNA repair protein RAD51 homolog 3 | 3.18E−03 | 42162.89 | O43502 | 1 | 2 | 2 |
| 18 | CA | Ig lambda chain V region | 4.35E−03 | 12372.02 | P04211 | 1 | 2 | 2 |
| 19 | BE | Keratin, type II | 4.19E−05 | 59831.25 | P04259 | 1 | 2 | 2 |
| 20 | CA | Cation-independent mannose-6-phosphate receptor | 5.66E−03 | 274097.6 | P11717 | 1 | 2 | 2 |
| 21 | CE | Macrophage scavenger receptor types I and II | 7.76E−04 | 49730.82 | P21757 | 1 | 2 | 2 |
| 22 | CD | Matrin-3 | 3.80E−03 | 94564.67 | P43243 | 1 | 2 | 2 |
| 23 | CE | Voltage-dependent anion-selective channel protein 2 | 4.14E−04 | 38068.68 | P45880 | 1 | 2 | 2 |
| 24 | BE | FLJ00268 protein (Fragment) | 2.78E−03 | 72805.3 | Q6ZML5 | 1 | 2 | 2 |
| 25 | CA | LAG1 longevity assurance homolog 2 | 1.46E−04 | 44847.38 | Q96G23 | 1 | 2 | 2 |
| 26 | CA | Serine/threonine-protein kinase | 8.12E−03 | 134655.6 | Q96J92 | 1 | 2 | 2 |
| 27 | CD | Hypothetical protein FLJ13940 | 2.89E−03 | 27528.79 | Q9H853 | 1 | 2 | 2 |
| 28 | B | Adenylate kinase isoenzyme 2 | 1.02E−03 | 26329.73 | P54819 | 2 | 1 | 2 |
| 29 | C | DAX1_HUMAN (P51843) Nuclear receptor 0B1 (Nuclear r [symbol] | 1.40E−03 | 51683.89 | P51843 | 1 | 1 | 2 |
| 30 | D | ZN473_HUMAN (Q8WTR7) Zinc finger protein 473 (Zinc f [symbol] | 2.47E−03 | 100118.1 | Q8WTR7 | 1 | 1 | 2 |
| 31 | B | CPSF3_HUMAN (Q9UKF6) Cleavage and polyadenylation [symbol] | 1.14E−03 | 77436.22 | Q9UKF6 | 1 | 1 | 2 |
| 32 | B | K2C5_HUMAN (P13647) Keratin, type II cytoskeletal 5 (Cy [symbol] | 7.34E−08 | 62409.08 | P13647 | 3 | 1 | 1 |
| 33 | B | CAH1_HUMAN (P00915) Carbonic anhydrase 1 (EC 4.2.1 [symbol] | 8.52E−08 | 28721.34 | P00915 | 2 | 1 | 1 |
| 34 | A | KNG1_HUMAN (P01042) Kininogen-1 precursor (Alpha-2- [symbol] | 2.12E−10 | 71900.1 | P01042 | 2 | 1 | 1 |
| 35 | B | K1C14_HUMAN (P02533) Keratin, type I cytoskeletal 14 ( [symbol] | 3.33E−06 | 51458.45 | P02533 | 2 | 1 | 1 |
| 36 | D | O00211 (O00211) HSLK (Ste20-related serineVthreonine [symbol] | 8.39E−03 | 138909.7 | O00211 | 1 | 1 | 1 |
| 37 | D | P3C2A_HUMAN (O00443) Phosphatidylinositol-4-phosph [symbol] | 7.07E−03 | 190616.2 | O00443 | 1 | 1 | 1 |
| 38 | B | SYT5_HUMAN (O00445) Synaptotagmin-5 (Synaptotagmi [symbol] | 3.71E−03 | 42873.54 | O00445 | 1 | 1 | 1 |
| 39 | A | CHD2_HUMAN (O14647) Chromodomain-helicase-DNA-b [symbol] | 7.04E−03 | 200437.6 | O14647 | 1 | 1 | 1 |
| 40 | C | MLL2_HUMAN (O14686) Myeloid/lymphoid or mixed-linea [symbol] | 8.21E−04 | 563835.8 | O14686 | 1 | 1 | 1 |
| 41 | E | O15042 (O15042) Hypothetical protein KIAA0332 (U2-ass [symbol] | 7.16E−04 | 118087.4 | O15042 | 1 | 1 | 1 |
| 42 | B | SNPH_HUMAN (O15079) Syntaphilin | 7.58E−03 | 57953.73 | O15079 | 1 | 1 | 1 |
| 43 | A | WDR46_HUMAN (O15213) WD-repeat protein 46 (WD-re [symbol] | 3.67E−03 | 67998.89 | O15213 | 1 | 1 | 1 |
| 44 | E | HAPIP_HUMAN (O60229) Huntingtin-associated protein-i [symbol] | 3.37E−03 | 192107.9 | O60229 | 1 | 1 | 1 |
| 45 | E | O60687 (O60687) Sushi-repeat-containing protein, X-linke [symbol] | 4.07E−03 | 52937.79 | O60687 | 1 | 1 | 1 |
| 46 | D | ZC11A_HUMAN (O75152) Zinc finger CCCH-type domain [symbol] | 4.33E−03 | 89076.06 | O75152 | 1 | 1 | 1 |
| 47 | C | O75198 (O75198) CDC37-like gene | 1.49E−03 | 26530.93 | O75198 | 1 | 1 | 1 |
| 48 | A | AVIL_HUMAN (O75366) Advillin (p92) | 8.01E−03 | 92028.35 | O75366 | 1 | 1 | 1 |
| 49 | C | MYCB2_HUMAN (O75592) Probable ubiquitin ligase prote [symbol] | 7.36E−03 | 509842.3 | O75592 | 1 | 1 | 1 |
| 50 | A | CMC1_HUMAN (O75746) Calcium-binding mitochondrial [symbol] | 3.32E−03 | 74709.01 | O75746 | 1 | 1 | 1 |
| 51 | E | MFN2_HUMAN (O95140) Transmembrane GTPase MFN2 [symbol] | 4.18E−03 | 86346.95 | O95140 | 1 | 1 | 1 |
| 52 | B | G6PE_HUMAN (O95479) GDH/6PGL endoplasmic bifunct [symbol] | 3.69E−03 | 88822.63 | O95479 | 1 | 1 | 1 |
| 53 | D | HPTR_HUMAN (P00739) Haptoglobin-related protein prec [symbol] | 5.05E−03 | 38982.66 | P00739 | 1 | 1 | 1 |
| 54 | A | CFAB_HUMAN (P00751) Complement factor B precursor [symbol] | 2.24E−04 | 85478.58 | P00751 | 1 | 1 | 1 |
| 55 | A | ASSY_HUMAN (P00966) Argininosuccinate synthase (EC [symbol] | 4.02E−03 | 46501.01 | P00966 | 1 | 1 | 1 |
| 56 | B | HRG_HUMAN (P04196) Histidine-rich glycoprotein precur [symbol] | 5.94E−04 | 59540.94 | P04196 | 1 | 1 | 1 |
| 57 | A | CFAI_HUMAN (P05156) Complement factor I precursor (E [symbol] | 2.93E−05 | 65676.66 | P05156 | 1 | 1 | 1 |
| 58 | D | ITB1_HUMAN (P05556) Integrin beta-1 precursor (Fibrone [symbol] | 7.50E−05 | 88406.98 | P05556 | 1 | 1 | 1 |
| 59 | A | KV2F_HUMAN (P06310) Ig kappa chain V-II region RPMI [symbol] | 1.78E−06 | 14697.37 | P06310 | 1 | 1 | 1 |
| 60 | B | K1C16_HUMAN (P08779) Keratin, type I cytoskeletal 16 ( [symbol] | 1.08E−06 | 51105.21 | P08779 | 1 | 1 | 1 |
| 61 | E | TAU_HUMAN (P10636) Microtubule-associated protein ta [symbol] | 7.47E−04 | 78698.83 | P10636 | 1 | 1 | 1 |
| 62 | D | PABP1_HUMAN (P11940) Polyadenylate-binding protein 1 [symbol] | 9.99E−03 | 70626.04 | P11940 | 1 | 1 | 1 |
| 63 | B | SCG2_HUMAN (P13521) Secretogranin-2 precursor (Secr [symbol] | 6.93E−03 | 70825.11 | P13521 | 1 | 1 | 1 |
| 64 | C | UCHL3_HUMAN (P15374) Ubiquitin carboxyl-terminal hyd [symbol] | 6.36E−03 | 26165.98 | P15374 | 1 | 1 | 1 |
| 65 | B | CBPE_HUMAN (P16870) Carboxypeptidase E precursor ( [symbol] | 1.65E−06 | 53117.17 | P16870 | 1 | 1 | 1 |
| 66 | B | AATC_HUMAN (P17174) Aspartate aminotransferase, cyt [symbol] | 9.28E−03 | 46087.54 | P17174 | 1 | 1 | 1 |
| 67 | B | A1ATR_HUMAN (P20848) Alpha-1-antitrypsin-related prot [symbol] | 7.19E−03 | 47860.97 | P20848 | 1 | 1 | 1 |
| 68 | E | RAE2_HUMAN (P26374) Rab proteins geranylgeranyltran [symbol] | 1.83E−03 | 74024.21 | P26374 | 1 | 1 | 1 |
| 69 | B | VGFR2_HUMAN (P35968) Vascular endothelial growth fa [symbol] | 1.56E−03 | 151430.5 | P35968 | 1 | 1 | 1 |
| 70 | B | CH3L1_HUMAN (P36222) Chitinase-3-like protein 1 precu [symbol] | 2.71E−06 | 42586.39 | P36222 | 1 | 1 | 1 |
| 71 | B | MAP1B_HUMAN (P46821) Microtubule-associated protein [symbol] | 5.22E−03 | 270451.8 | P46821 | 1 | 1 | 1 |
| 72 | B | PCY1A_HUMAN (P49585) Choline-phosphate cytidylyltra [symbol] | 3.65E−03 | 41705.8 | P49585 | 1 | 1 | 1 |
| 73 | E | CEBPD_HUMAN (P49716) CCAAT/enhancer binding prot [symbol] | 6.61E−03 | 28461.45 | P49716 | 1 | 1 | 1 |
| 74 | D | CCR6_HUMAN (P51684) C—C chemokine receptor type 6 [symbol] | 7.26E−03 | 42465.93 | P51684 | 1 | 1 | 1 |
| 75 | C | EPHA5_HUMAN (P54756) Ephrin type-A receptor 5 precu [symbol] | 7.74E−03 | 114710.3 | P54756 | 1 | 1 | 1 |
| 76 | A | PSD4_HUMAN (P55036) 26S proteasome non-ATPase re [symbol] | 6.62E−04 | 40711.21 | P55036 | 1 | 1 | 1 |
| 77 | B | B2MG_HUMAN (P61769) Beta-2-microglobulin precursor [ [symbol] | 3.51E−07 | 13705.91 | P61769 | 1 | 1 | 1 |
| 78 | A | GBB1_HUMAN (P62873) Guanine nucleotide-binding prot [symbol] | 1.27E−03 | 37221.98 | P62873 | 1 | 1 | 1 |
| 79 | D | CXAR_HUMAN (P78310) Coxsackievirus and adenovirus [symbol] | 1.03E−03 | 40004.39 | P78310 | 1 | 1 | 1 |
| 80 | A | GALT1_HUMAN (Q10472) Polypeptide N-acetylgalactosar [symbol] | 3.87E−03 | 64177.48 | Q10472 | 1 | 1 | 1 |
| 81 | B | PTN13_HUMAN (Q12923) Tyrosine-protein phosphatase, [symbol] | 3.13E−05 | 276731.3 | Q12923 | 1 | 1 | 1 |
| 82 | C | DLG1_HUMAN (Q12959) Presynaptic protein SAP97 (Syn [symbol] | 6.56E−03 | 100293.1 | Q12959 | 1 | 1 | 1 |
| 83 | A | AKAP6_HUMAN (Q13023) A-kinase anchor protein 6 (Pro [symbol] | 1.63E−03 | 256503.6 | Q13023 | 1 | 1 | 1 |
| 84 | A | ATM_HUMAN (Q13315) Serine-protein kinase ATM (EC 2. [symbol] | 4.51E−03 | 350417.1 | Q13315 | 1 | 1 | 1 |
| 85 | B | ROCK1_HUMAN (Q13464) Rho-associated protein kinase [symbol] | 1.42E−03 | 158074.9 | Q13464 | 1 | 1 | 1 |
| 86 | A | ENPP2_HUMAN (Q13822) Ectonucleotide pyrophosphata [symbol] | 5.80E−10 | 98939.61 | Q13822 | 1 | 1 | 1 |
| 87 | D | SMC1A_HUMAN (Q14683) Structural maintenance of chr [symbol] | 9.76E−04 | 143143.6 | Q14683 | 1 | 1 | 1 |
| 88 | C | NOLC1_HUMAN (Q14978) Nucleolar phosphoprotein p13 [symbol] | 1.80E−03 | 73676.72 | Q14978 | 1 | 1 | 1 |
| 89 | A | PLEC1_HUMAN (Q15149) Plectin 1 (PLTN) (PCN) (Hemi [symbol] | 2.61E−04 | 531411.9 | Q15149 | 1 | 1 | 1 |
| 90 | E | K1H1_HUMAN (Q15323) Keratin, type I cuticular Ha1 (Hai [symbol] | 1.36E−03 | 47202.1 | Q15323 | 1 | 1 | 1 |
| 91 | A | NCOA1_HUMAN (Q15788) Nuclear receptor coactivator 1 [symbol] | 4.14E−04 | 156641.7 | Q15788 | 1 | 1 | 1 |
| 92 | A | Q15813 (Q15813) Beta-tubulin cofactor E (Tubulin-specific [symbol] | 5.90E−03 | 59309.08 | Q15813 | 1 | 1 | 1 |
| 93 | A | SCN9A_HUMAN (Q15858) Sodium channel protein type I [symbol] | 6.73E−03 | 226193.9 | Q15858 | 1 | 1 | 1 |
| 94 | B | Q495C9 (Q495C9) Hypothetical protein LOC149134 | 6.59E−03 | 13623.18 | Q495C9 | 1 | 1 | 1 |
| 95 | A | Q4G0M1 (Q4G0M1) FLJ37034 protein (Fragment) | 6.80E−03 | 14453.34 | Q4G0M1 | 1 | 1 | 1 |
| 96 | D | Q567P4 (Q567P4) Hypothetical protein (Fragment) | 2.58E−03 | 34603.27 | Q567P4 | 1 | 1 | 1 |
| 97 | A | LRC10_HUMAN (Q5BKY1) Leucine-rich repeat-containing [symbol] | 9.03E−04 | 31621.81 | Q5BKY1 | 1 | 1 | 1 |
| 98 | B | Q5H9K7 (Q5H9K7) Novel protein (Fragment) | 6.32E−03 | 38685.59 | Q5H9K7 | 1 | 1 | 1 |
| 99 | D | Q5H9K8 (Q5H9K8) Armadillo repeat containing, X-linked 4 [symbol] | 7.72E−03 | 36484.3 | Q5H9K8 | 1 | 1 | 1 |
| 100 | C | Q5JRL1 (Q5JRL1) Novel protein | 6.73E−03 | 30747.36 | Q5JRL1 | 1 | 1 | 1 |
| 101 | E | Q5T364 (Q5T364) OTTHUMP00000022702 | 4.05E−03 | 101941 | Q5T364 | 1 | 1 | 1 |
| 102 | C | Q5T3B9 (Q5T3B9) Novel protein | 4.46E−03 | 45993.33 | Q5T3B9 | 1 | 1 | 1 |
| 103 | D | Q5TBP2 (Q5TBP2) Retinoblastoma-associated factor 600 [symbol] | 4.66E−03 | 106303.1 | Q5TBP2 | 1 | 1 | 1 |
| 104 | B | Q5VWL1 (Q5VWL1) Membrane-associated guanylate kina [symbol] | 7.51E−03 | 162847.8 | Q5VWL1 | 1 | 1 | 1 |
| 105 | C | Q5XLJ0 (Q5XLJ0) AIDA1C transcript variant 4 | 9.99E−03 | 48400.84 | Q5XLJ0 | 1 | 1 | 1 |
| 106 | E | Q6DN23 (Q6DN23) Platelet phospholipase A2 | 4.11E−03 | 16135.78 | Q6DN23 | 1 | 1 | 1 |
| 107 | C | Q6IAM5 (Q6IAM5) SLC27A6 protein | 3.12E−03 | 70042.55 | Q6IAM5 | 1 | 1 | 1 |
| 108 | B | Q6JIC5 (Q6JIC5) Cementum attachment protein | 9.87E−03 | 14910.42 | Q6JIC5 | 1 | 1 | 1 |
| 109 | A | Q6MZX7 (Q6MZX7) Hypothetical protein DKFZp686M242 [symbol] | 1.00E−30 | 52387.13 | Q6MZX7 | 1 | 1 | 1 |
| 110 | D | Q6P2C5 (Q6P2C5) PHF2 protein | 5.50E−03 | 35720.63 | Q6P2C5 | 1 | 1 | 1 |
| 111 | E | Q6P528 (Q6P528) ASPN protein | 8.71E−03 | 43863.48 | Q6P528 | 1 | 1 | 1 |
| 112 | E | POTE2_HUMAN (Q6S8J3) Prostate, ovary, testis express [symbol] | 6.93E−03 | 80701.5 | Q6S8J3 | 1 | 1 | 1 |
| 113 | E | Q6UXZ8 (Q6UXZ8) FVSY9334 | 7.07E−04 | 24230.68 | Q6UXZ8 | 1 | 1 | 1 |
| 114 | A | Q6ZN08 (Q6ZN08) Hypothetical protein FLJ16537 | 4.37E−04 | 38813.18 | Q6ZN08 | 1 | 1 | 1 |
| 115 | D | Q6ZR14 (Q6ZR14) Hypothetical protein FLJ46736 | 8.79E−03 | 126662.9 | Q6ZR14 | 1 | 1 | 1 |
| 116 | E | Q6ZSK5 (Q6ZSK5) Hypothetical protein FLJ45433 | 6.04E−03 | 15222.69 | Q6ZSK5 | 1 | 1 | 1 |
| 117 | B | Q6ZSY5 (Q6ZSY5) Hypothetical protein FLJ45123 | 7.90E−03 | 42154.05 | Q6ZSY5 | 1 | 1 | 1 |
| 118 | A | Q6ZVQ3 (Q6ZVQ3) Hypothetical protein FLJ42220 | 1.14E−03 | 17595.21 | Q6ZVQ3 | 1 | 1 | 1 |
| 119 | D | Q6ZW49 (Q6ZW49) Hypothetical protein FLJ41606 | 3.89E−03 | 117615.6 | Q6ZW49 | 1 | 1 | 1 |
| 120 | C | Q70AK8 (Q70AK8) Ankyrin-repeat-ARM domain protein (F [symbol] | 8.67E−04 | 40496.33 | Q70AK8 | 1 | 1 | 1 |
| 121 | A | Q70CQ2 (Q70CQ2) Ubiquitin-specific proteinase 34 | 9.41E−03 | 386873.7 | Q70CQ2 | 1 | 1 | 1 |
| 122 | C | Q76E79 (Q76E79) Mitochondrial methionyl-tRNA syntheta [symbol] | 2.95E−03 | 66584.84 | Q76E79 | 1 | 1 | 1 |
| 123 | C | Q7L7Q2 (Q7L7Q2) BLOM7 beta (Novel protein) | 1.49E−04 | 59101.39 | Q7L7Q2 | 1 | 1 | 1 |
| 124 | D | Q7Z3P5 (Q7Z3P5) Hypothetical protein DKFZp686P13218 [symbol] | 5.90E−04 | 30940.46 | Q7Z3P5 | 1 | 1 | 1 |
| 125 | A | NPHP3_HUMAN (Q7Z494) Nephrocystin-3 | 5.74E−03 | 150768.9 | Q7Z494 | 1 | 1 | 1 |
| 126 | E | Q86VG6 (Q86VG6) Hypothetical protein | 3.43E−03 | 11523.95 | Q86VG6 | 1 | 1 | 1 |
| 127 | C | TXND2_HUMAN (Q86VQ3) Thioredoxin domain-containin [symbol] | 7.89E−04 | 60424.09 | Q86VQ3 | 1 | 1 | 1 |
| 128 | D | NAL14_HUMAN (Q86W24) NACHT-, LRR- and PYD-cont [symbol] | 1.88E−03 | 124651.5 | Q86W24 | 1 | 1 | 1 |
| 129 | E | Q86WW8 (Q86WW8) Hypothetical protein MGC52110 (H [symbol] | 4.59E−03 | 8370.071 | Q86WW8 | 1 | 1 | 1 |
| 130 | D | Q86Y92 (Q86Y92) Similar to KIAA0922 protein (Fragment) [symbol] | 2.24E−03 | 154618.6 | Q86Y92 | 1 | 1 | 1 |
| 131 | D | Q8IV73 (Q8IV73) Guanine nucleotide-releasing factor 2, is [symbol] | 3.40E−03 | 122658.6 | Q8IV73 | 1 | 1 | 1 |
| 132 | B | Q8IVL1 (Q8IVL1) Steerin2 protein | 2.98E−03 | 267950.3 | Q8IVL1 | 1 | 1 | 1 |
| 133 | B | Q8IVM4 (Q8IVM4) Hypothetical protein | 8.10E−03 | 6014.06 | Q8IVM4 | 1 | 1 | 1 |
| 134 | A | FA20C_HUMAN (Q8IXL6) Protein FAM20C precursor | 5.31E−03 | 64405.57 | Q8IXL6 | 1 | 1 | 1 |
| 135 | E | Q8IY66 (Q8IY66) DNA ligase IV (Ligase IV, DNA, ATP-de [symbol] | 7.76E−03 | 103904 | Q8IY66 | 1 | 1 | 1 |
| 136 | D | GP115_HUMAN (Q8IZF3) Probable G-protein coupled rec [symbol] | 5.14E−04 | 83775.66 | Q8IZF3 | 1 | 1 | 1 |
| 137 | B | Q8IZQ1 (Q8IZQ1) ALFY | 6.15E−03 | 394992 | Q8IZQ1 | 1 | 1 | 1 |
| 138 | D | AHI1_HUMAN (Q8N157) Jouberin (Abelson helper integra [symbol] | 2.76E−03 | 137029 | Q8N157 | 1 | 1 | 1 |
| 139 | C | Q8N1K5 (Q8N1K5) Hypothetical protein FLJ40584 | 5.82E−03 | 73625.31 | Q8N1K5 | 1 | 1 | 1 |
| 140 | D | Q8N3I3 (Q8N3I3) Hypothetical protein DKFZp761P18121 | 3.18E−03 | 81550.7 | Q8N3I3 | 1 | 1 | 1 |
| 141 | A | Q8N543 (Q8N543) Hypothetical protein FLJ10826 | 1.90E−03 | 63206.21 | Q8N543 | 1 | 1 | 1 |
| 142 | A | Q8N998 (Q8N998) Hypothetical protein FLJ38159 | 7.01E−03 | 43781.59 | Q8N998 | 1 | 1 | 1 |
| 143 | D | Q8N9S2 (Q8N9S2) Hypothetical protein FLJ36635 | 5.25E−03 | 28973.04 | Q8N9S2 | 1 | 1 | 1 |
| 144 | E | Q8NAJ6 (Q8NAJ6) Hypothetical protein FLJ35251 | 1.24E−03 | 81745.5 | Q8NAJ6 | 1 | 1 | 1 |
| 145 | A | Q8NCU4 (Q8NCU4) Hypothetical protein | 9.83E−03 | 110499 | Q8NCU4 | 1 | 1 | 1 |
| 146 | E | Q8NDA2 (Q8NDA2) Hypothetical protein DKFZp434P0216 [symbol] | 7.52E−04 | 143799.3 | Q8NDA2 | 1 | 1 | 1 |
| 147 | A | Q8TAN9 (Q8TAN9) Oxoglutarate dehydrogenase-like | 6.91E−03 | 114423.7 | Q8TAN9 | 1 | 1 | 1 |
| 148 | E | Q8TBB5 (Q8TBB5) Kelch domain containing 4 | 1.01E−03 | 57855.43 | Q8TBB5 | 1 | 1 | 1 |
| 149 | A | Q8TBZ0 (Q8TBZ0) Hypothetical protein KM-HN-1 | 6.67E−04 | 96664.98 | Q8TBZ0 | 1 | 1 | 1 |
| 150 | B | Q8TC05 (Q8TC05) Mdm4, transformed 3T3 cell double mi [symbol] | 5.19E−03 | 80695.09 | Q8TC05 | 1 | 1 | 1 |
| 151 | B | CHD6_HUMAN (Q8TD26) Chromodomain-helicase-DNA- [symbol] | 6.52E−03 | 305218.9 | Q8TD26 | 1 | 1 | 1 |
| 152 | B | PAR3L_HUMAN (Q8TEW8) Amyotrophic lateral sclerosis [symbol] | 9.52E−03 | 132412.5 | Q8TEW8 | 1 | 1 | 1 |
| 153 | E | Q8WVY7 (Q8WVY7) Ubiquitin-like domain containing CT [symbol] | 8.32E−04 | 36781.22 | Q8WVY7 | 1 | 1 | 1 |
| 154 | A | ANKR7_HUMAN (Q92527) Ankyrin repeat domain protein | 8.25E−03 | 22573.66 | Q92527 | 1 | 1 | 1 |
| 155 | E | Q92735 (Q92735) FMI protein | 4.05E−03 | 161492.6 | Q92735 | 1 | 1 | 1 |
| 156 | C | SNPC3_HUMAN (Q92966) snRNA-activating protein comp [symbol] | 9.07E−03 | 46722.65 | Q92966 | 1 | 1 | 1 |
| 157 | A | IRF7_HUMAN (Q92985) Interferon regulatory factor 7 (IRF [symbol] | 7.33E−04 | 54244.25 | Q92985 | 1 | 1 | 1 |
| 158 | D | Q96AG4 (Q96AG4) Hypothetical protein PRO1855 | 9.06E−03 | 34908.9 | Q96AG4 | 1 | 1 | 1 |
| 159 | A | ISG20_HUMAN (Q96AZ6) Interferon-stimulated gene 20 k [symbol] | 9.06E−05 | 20350.62 | Q96AZ6 | 1 | 1 | 1 |
| 160 | C | GMCL1_HUMAN (Q96IK5) Germ cell-less protein-like 1 | 7.74E−03 | 58647.47 | Q96IK5 | 1 | 1 | 1 |
| 161 | A | KAD7_HUMAN (Q96M32) Putative adenylate kinase 7 (EC [symbol] | 9.64E−03 | 82620.65 | Q96M32 | 1 | 1 | 1 |
| 162 | A | DB118_HUMAN (Q96PH6) Beta-defensin 118 precursor ( [symbol] | 5.68E−03 | 13604.79 | Q96PH6 | 1 | 1 | 1 |
| 163 | D | VPS35_HUMAN (Q96QK1) Vacuolar protein sorting 35 (V [symbol] | 4.26E−03 | 91649.07 | Q96QK1 | 1 | 1 | 1 |
| 164 | E | MAGI1_HUMAN (Q96QZ7) Membrane associated guanyla [symbol] | 7.80E−03 | 164540.2 | Q96QZ7 | 1 | 1 | 1 |
| 165 | D | MCCA_HUMAN (Q96RQ3) Methylcrotonoyl-CoA carboxyla [symbol] | 3.52E−03 | 80382.04 | Q96RQ3 | 1 | 1 | 1 |
| 166 | E | NIBL_HUMAN (Q96TA1) Niban-like protein (Meg-3) | 6.21E−03 | 82631.11 | Q96TA1 | 1 | 1 | 1 |
| 167 | C | SPS2_HUMAN (Q99611) Selenide, water dikinase 2 (EC 2 [symbol] | 2.61E−03 | 47227.85 | Q99611 | 1 | 1 | 1 |
| 168 | D | KIF2C_HUMAN (Q99661) Kinesin-like protein KIF2C (Mito [symbol] | 5.02E−03 | 81261.21 | Q99661 | 1 | 1 | 1 |
| 169 | E | CLP24_HUMAN (Q9BSN7) Claudin-like protein 24 | 2.48E−03 | 24523.55 | Q9BSN7 | 1 | 1 | 1 |
| 170 | E | DATF1_HUMAN (Q9BTC0) Death-associated transcription [symbol] | 7.61E−03 | 129070.5 | Q9BTC0 | 1 | 1 | 1 |
| 171 | E | CHST6_HUMAN (Q9GZX3) Carbohydrate sulfotransferase [symbol] | 5.32E−03 | 44071.2 | Q9GZX3 | 1 | 1 | 1 |
| 172 | C | TNKS2_HUMAN (Q9H2K2) Tankyrase 2 (EC 2.4.2.30) (TA [symbol] | 1.90E−05 | 126838.5 | Q9H2K2 | 1 | 1 | 1 |
| 173 | C | Q9H2M8 (Q9H2M8) DC28 | 8.59E−03 | 64005.01 | Q9H2M8 | 1 | 1 | 1 |
| 174 | E | SMRCD_HUMAN (Q9H4L7) SWI/SNF-related, matrix ass [symbol] | 7.44E−03 | 117299.9 | Q9H4L7 | 1 | 1 | 1 |
| 175 | C | Q9H9B0 (Q9H9B0) Hypothetical protein FLJ12883 | 9.96E−03 | 91169.25 | Q9H9B0 | 1 | 1 | 1 |
| 176 | A | Q9H9B6 (Q9H9B6) Hypothetical protein FLJ12873 | 4.63E−03 | 71307.3 | Q9H9B6 | 1 | 1 | 1 |
| 177 | D | LPHN3_HUMAN (Q9HAR2) Latrophilin-3 precursor (Calci [symbol] | 9.90E−03 | 161709.3 | Q9HAR2 | 1 | 1 | 1 |
| 178 | A | MAGE1_HUMAN (Q9HCI5) Melanoma-associated antigen | 6.56E−03 | 103189.7 | Q9HCI5 | 1 | 1 | 1 |
| 179 | D | OLFL3_HUMAN (Q9NRN5) Olfactomedin-like protein 3 pr [symbol] | 1.86E−03 | 45981.32 | Q9NRN5 | 1 | 1 | 1 |
| 180 | D | TB22B_HUMAN (Q9NU19) TBC1 domain family member 2 [symbol] | 3.31E−03 | 59044.32 | Q9NU19 | 1 | 1 | 1 |
| 181 | B | CT038_HUMAN (Q9NUV7) Protein C20orf38 | 7.71E−03 | 19679.8 | Q9NUV7 | 1 | 1 | 1 |
| 182 | A | Q9NXE3 (Q9NXE3) Hypothetical protein FLJ20298 | 8.80E−04 | 26243.39 | Q9NXE3 | 1 | 1 | 1 |
| 183 | E | THUM1_HUMAN (Q9NXG2) THUMP domain containing p [symbol] | 7.48E−03 | 39290.82 | Q9NXG2 | 1 | 1 | 1 |
| 184 | B | TE2IP_HUMAN (Q9NYB0) Telomeric repeat binding factor [symbol] | 1.44E−03 | 44232.88 | Q9NYB0 | 1 | 1 | 1 |
| 185 | E | ZN226_HUMAN (Q9NYT6) Zinc finger protein 226 | 4.95E−03 | 91861.71 | Q9NYT6 | 1 | 1 | 1 |
| 186 | A | GSCR1_HUMAN (Q9NZM4) Glioma tumor suppressor can [symbol] | 6.54E−03 | 152898.4 | Q9NZM4 | 1 | 1 | 1 |
| 187 | B | HCN3_HUMAN (Q9P1Z3) Potassium/sodium hyperpolariz [symbol] | 5.36E−03 | 85977.23 | Q9P1Z3 | 1 | 1 | 1 |
| 188 | A | CEP72_HUMAN (Q9P209) Centrosomal protein of 72 kDa [symbol] | 1.96E−03 | 71673.01 | Q9P209 | 1 | 1 | 1 |
| 189 | A | CING_HUMAN (Q9P2M7) Cingulin | 5.06E−03 | 136303.7 | Q9P2M7 | 1 | 1 | 1 |
| 190 | D | SL9A2_HUMAN (Q9UBY0) Sodium/hydrogen exchanger 2 [symbol] | 6.23E−04 | 91461.34 | Q9UBY0 | 1 | 1 | 1 |
| 191 | E | Q9UJU1 (Q9UJU1) Cytovillin 2 (Fragment) | 8.20E−03 | 16251.62 | Q9UJU1 | 1 | 1 | 1 |
| 192 | C | STML2_HUMAN (Q9UJZ1) Stomatin-like protein 2 (SLP-2) [symbol] | 2.48E−03 | 38510.22 | Q9UJZ1 | 1 | 1 | 1 |
| 193 | C | MYH13_HUMAN (Q9UKX3) Myosin-13 (Myosin heavy cha [symbol] | 5.60E−03 | 223539.2 | Q9UKX3 | 1 | 1 | 1 |
| 194 | D | TTC7A_HUMAN (Q9ULT0) Tetratricopeptide repeat protei [symbol] | 6.23E−03 | 96123.27 | Q9ULT0 | 1 | 1 | 1 |
| 195 | E | ALK_HUMAN (Q9UM73) ALK tyrosine kinase receptor pre [symbol] | 9.57E−03 | 176304.4 | Q9UM73 | 1 | 1 | 1 |
| 196 | D | ICAM5_HUMAN (Q9UMF0) Intercellular adhesion molecul [symbol] | 2.21E−03 | 97270.1 | Q9UMF0 | 1 | 1 | 1 |
| 197 | C | SOX13_HUMAN (Q9UN79) SOX-13 protein (Type 1 diabe [symbol] | 7.41E−03 | 98718.72 | Q9UN79 | 1 | 1 | 1 |
| 198 | E | CNTN6_HUMAN (Q9UQ52) Contactin 6 precursor (Neural [symbol] | 5.55E−03 | 113885.1 | Q9UQ52 | 1 | 1 | 1 |
| 199 | A | NALDL_HUMAN (Q9UQQ1) N-acetylated-alpha-linked aci [symbol] | 1.21E−03 | 80574.27 | Q9UQQ1 | 1 | 1 | 1 |
| 200 | D | CG141_HUMAN (Q9Y3E0) UPF0198 protein CGI-141 | 5.94E−05 | 15415.4 | Q9Y3E0 | 1 | 1 | 1 |
| 201 | D | ZN337_HUMAN (Q9Y3M9) Zinc finger protein 337 | 4.48E−03 | 86819.26 | Q9Y3M9 | 1 | 1 | 1 |
| 202 | C | TLN1_HUMAN (Q9Y490) Talin-1 | 3.76E−03 | 269596.3 | Q9Y490 | 1 | 1 | 1 |
| 203 | C | PCDGB_HUMAN (Q9Y5H2) Protocadherin gamma A11 pr [symbol] | 9.51E−03 | 101480.6 | Q9Y5H2 | 1 | 1 | 1 |
| 204 | A | WIF1_HUMAN (Q9Y5W5) Wnt inhibitory factor 1 precurso [symbol] | 5.15E−03 | 41499.8 | Q9Y5W5 | 1 | 1 | 1 |
| 205 | B | S12A7_HUMAN (Q9Y666) Solute carrier family 12 membe [symbol] | 1.59E−04 | 119072.7 | Q9Y666 | 1 | 1 | 1 |
| 206 | D | SNCAP_HUMAN (Q9Y6H5) Synphilin-1 (Alpha-synuclein-i [symbol] | 5.97E−03 | 100319 | Q9Y6H5 | 1 | 1 | 1 |

| Protein | Location | Function |
|---|---|---|
| LOC131076 protein | unknown | function unknown, coiled coiled domain |
| T-cell activation Rho GTPase-activating protein | cytoplasm | GTP-ase activator, T cell activation |
| Huntingtin interacting protein E | cytoplasm | signal transduction |
| Synaptotagmin-5 (Synaptotagmin V) (SytV) | Synaptic | Calcium dependent exocytosis |
| Huntingtin-associated protein-interacting protein | cytoplasm | signal transduction |
| VGFR2_Vascular endothelial growth factor receptor 2 precursor | membrane | angiogenesis, vascular permeability |
| Presynaptic protein SAP97 (Synapse-associated protein 97) | cytoskeleton | scaffolding protein |
| Retinoblastoma-associated factor 600 (RBAF600) (Fragment) | nucleus | possible chromatin remodeling |

TABLE 8
Proteins identified uniquely in Macular Hole diagnosed patients (5 patients, 5 pts × 5 runs = 25 runs total for study)
SEQUEST filters: p prob < 0.01; Xcorr vs charge state: 1.5 for [M + H]1+, 2.0 for [M + 2H]2+, 2.5 for [M + 3H]3+; DCn > 0.1

| | Pathol- | Pa- | | | | | Peptide | MH | TOT |
|---|---|---|---|---|---|---|---|---|---|
| | ogy | tients | Reference | P (pro) | MW | Accession | (Hits) | Count | ID |
| 1 | MH | ILH | Protein KIAA1683 | 2.65E−04 | 127611.8 | Q9H0B3 | 1 | 3 | 9 |
| 2 | MH | FH | Apolipoprotein A-IV precursor | 4.69E−12 | 45343.52 | P06727 | 3 | 2 | 5 |
| 3 | MH | HF | KIAA0425 protein | 2.12E−04 | 141056.5 | O43308 | 1 | 2 | 5 |
| 4 | MH | LH | MYH7B protein | 5.17E−04 | 114574.5 | Q96I57 | 1 | 2 | 5 |
| 5 | MH | GI | Sortilin-related receptor precursor | 2.94E−04 | 248280 | Q92673 | 1 | 2 | 4 |
| 6 | MH | G | Ig kappa chain V-II region | 4.45E−07 | 12668.32 | P01614 | 1 | 1 | 4 |
| 7 | MH | G | Ig kappa chain V-II region MIL | 4.45E−07 | 12048.05 | P01616 | 1 | 1 | 4 |
| 8 | MH | F | Piccolo protein (Aczonin) | 4.86E−04 | 566313.1 | Q9Y6V0 | 1 | 1 | 4 |
| 9 | MH | FGH | Alpha-1B-glycoprotein precursor | 1.32E−09 | 54238.7 | P04217 | 1 | 3 | 3 |
| 10 | MH | LIH | Neuronal membrane glycoprotein M6-a | 4.39E−03 | 31188.39 | P51674 | 1 | 3 | 3 |
| 11 | MH | L | Nuclear pore complex protein Nup153 | 5.51E−03 | 153793.4 | P49790 | 1 | 1 | 3 |
| 12 | MH | H | Secreted frizzled-related protein 3 precursor | 5.30E−06 | 36230.29 | Q92765 | 1 | 1 | 3 |
| 13 | MH | IL | A-kinase anchor protein 3 | 2.47E−05 | 94676.41 | O75969 | 1 | 2 | 2 |
| 14 | MH | GI | Telomeric repeat binding factor 1 | 2.43E−03 | 50313.11 | P54274 | 1 | 2 | 2 |
| 15 | MH | IH | Kinesin-like protein KIF1A | 3.48E−03 | 190963.1 | Q12756 | 1 | 2 | 2 |
| 16 | MH | IL | UDP-N-acetylhexosamine pyrophosphorylase | 2.88E−03 | 58731.98 | Q16222 | 1 | 2 | 2 |
| 17 | MH | LH | RIF1_HUMAN (Q5UIP0) Telomere-associated protein [symbol] | 2.56E−03 | 274293 | Q5UIP0 | 1 | 2 | 2 |
| 18 | MH | GH | Q6P4H8 (Q6P4H8) Hypothetical protein LOC134145 | 3.97E−03 | 26123.47 | Q6P4H8 | 1 | 2 | 2 |
| 19 | MH | HL | Q8IV47 (Q8IV47) Fibromodulin, | 6.22E−04 | 43151.57 | Q8IV47 | 1 | 2 | 2 |
| 20 | MH | LG | Q8N9V7 (Q8N9V7) Hypothetical protein FLJ36157 | 6.30E−04 | 77526.28 | Q8N9V7 | 1 | 2 | 2 |
| 21 | MH | HG | LIPB2_HUMAN (Q8ND30) Liprin-beta 2 (Protein tyrosi [symbol] | 3.34E−03 | 98383.37 | Q8ND30 | 1 | 2 | 2 |
| 22 | MH | GF | SYNE1_HUMAN (Q8NF91) Nesprin-1 (Nuclear envelo [symbol] | 1.60E−03 | 1010433 | Q8NF91 | 1 | 2 | 2 |
| 23 | MH | LH | K1849_HUMAN (Q96JH8) Protein KIAA1849 | 6.35E−03 | 116630 | Q96JH8 | 1 | 2 | 2 |
| 24 | MH | LH | GGA2_HUMAN (Q9UJY4) ADP-ribosylation factor bind | 3.44E−03 | 67133.75 | Q9UJY4 | 1 | 2 | 2 |
| 25 | MH | HL | Q9ULH9 (Q9ULH9) KIAA1241 protein (Fragment) | 1.73E−05 | 97158.45 | Q9ULH9 | 1 | 2 | 2 |
| 26 | MH | LH | CT004_HUMAN (Q9Y312) Protein C20orf4 | 5.11E−03 | 43444.13 | Q9Y312 | 1 | 2 | 2 |
| 27 | MH | GF | KIF3A_HUMAN (Q9Y496) Kinesin-like protein KIF3A ( [symbol] | 8.14E−04 | 80335.11 | Q9Y496 | 1 | 2 | 2 |
| 28 | MH | I | CAC1G_HUMAN (O43497) Voltage-dependent T-type [symbol] | 2.51E−03 | 262301.1 | O43497 | 1 | 1 | 2 |
| 29 | MH | I | GLU2B_HUMAN (P14314) Glucosidase II beta subunit | 1.97E−03 | 59258.88 | P14314 | 1 | 1 | 2 |
| 30 | MH | F | HBG1_HUMAN (P69891) Hemoglobin gamma-1 subun | 8.59E−03 | 15999.27 | P69891 | 1 | 1 | 2 |
| 31 | MH | F | APXL_HUMAN (Q13796) Apical-like protein (APXL pro | 1.55E−03 | 176302 | Q13796 | 1 | 1 | 2 |
| 32 | MH | I | UBP10_HUMAN (Q14694) Ubiquitin carboxyl-terminal [symbol] | 1.05E−03 | 87079.84 | Q14694 | 1 | 1 | 2 |
| 33 | MH | I | Q15394 (Q15394) KIAA0005 protein (Fragment) | 6.56E−04 | 48615.16 | Q15394 | 1 | 1 | 2 |
| 34 | MH | I | Q4V9L7 (Q4V9L7) NGRN protein (Fragment) | 4.36E−03 | 31332.13 | Q4V9L7 | 1 | 1 | 2 |
| 35 | MH | I | Q53SW1 (Q53SW1) Hypothetical protein FLJ20254 | 2.53E−03 | 48539.34 | Q53SW1 | 1 | 1 | 2 |
| 36 | MH | H | Q5BLQ2 (Q5BLQ2) Mucin | 5.22E−03 | 289658.2 | Q5BLQ2 | 1 | 1 | 2 |
| 37 | MH | F | Q5JRC3 (Q5JRC3) RPEL repeat containing 1 (Fragme | 4.33E−03 | 25543.2 | Q5JRC3 | 1 | 1 | 2 |
| 38 | MH | F | Q5T2J8 (Q5T2J8) OTTHUMP00000018139 (Fragment | 1.44E−03 | 97147.08 | Q5T2J8 | 1 | 1 | 2 |
| 39 | MH | G | Q65ZC8 (Q65ZC8) Single-chain Fv (Fragment) | 3.05E−05 | 26110.55 | Q65ZC8 | 1 | 1 | 2 |
| 40 | MH | I | VPS36_HUMAN (Q86VN1) Vacuolar protein sorting pr [symbol] | 1.09E−03 | 43788.79 | Q86VN1 | 1 | 1 | 2 |
| 41 | MH | I | RYR2_HUMAN (Q92736) Ryanodine receptor 2 (Cardi [symbol] | 6.98E−03 | 564136.1 | Q92736 | 1 | 1 | 2 |
| 42 | MH | I | TOP1M_HUMAN (Q969P6) DNA topoisomerase I, mit [symbol] | 5.03E−03 | 69828.34 | Q969P6 | 1 | 1 | 2 |
| 43 | MH | F | Q96EK0 (Q96EK0) Hypothetical protein MGC4655 | 1.44E−03 | 41865.86 | Q96EK0 | 1 | 1 | 2 |
| 44 | MH | F | Q9BRQ8 (Q9BRQ8) Apoptosis-inducing factor (AIF)-lik | 2.48E−03 | 40501.28 | Q9BRQ8 | 1 | 1 | 2 |
| 45 | MH | I | Q9BS34 (Q9BS34) Zinc finger protein 670 (Hypothetic [symbol] | 7.16E−03 | 44574.27 | Q9BS34 | 1 | 1 | 2 |
| 46 | MH | I | PLEA5_HUMAN (Q9HAU0) Pleckstrin homology domai | 3.50E−03 | 127384.5 | Q9HAU0 | 1 | 1 | 2 |
| 47 | MH | H | Q5VXU2 (Q5VXU2) Propionyl Coenzyme A carboxylas [symbol] | 1.34E−04 | 80235.37 | Q5VXU2 | 2 | 1 | 1 |
| 48 | MH | G | SGK1_HUMAN (O00141) Serine/threonine-protein kina | 1.08E−03 | 48925.35 | O00141 | 1 | 1 | 1 |
| 49 | MH | F | O14913 (O14913) Kruppel-associated box protein | 2.39E−03 | 52045.66 | O14913 | 1 | 1 | 1 |
| 50 | MH | G | HAT1_HUMAN (O14929) Histone acetyltransferase typ | 1.43E−03 | 49480.84 | O14929 | 1 | 1 | 1 |
| 51 | MH | F | O15014 (O15014) KIAA0295 protein (Fragment) | 2.79E−03 | 106382.5 | O15014 | 1 | 1 | 1 |
| 52 | MH | L | PDZK3_HUMAN (O15018) PDZ domain containing pro | 2.91E−03 | 301423.4 | O15018 | 1 | 1 | 1 |
| 53 | MH | F | DMN_HUMAN (O15061) Desmuslin | 2.87E−03 | 172662.5 | O15061 | 1 | 1 | 1 |
| 54 | MH | G | SOX12_HUMAN (O15370) SOX-12 protein (SOX-22 pr | 1.92E−03 | 34279.67 | O15370 | 1 | 1 | 1 |
| 55 | MH | F | O43719 (O43719) HIV TAT specific factor 1 | 2.48E−03 | 85800.38 | O43719 | 1 | 1 | 1 |
| 56 | MH | F | KPRB_HUMAN (O60256) Phosphoribosyl pyrophospha | 3.63E−05 | 40899.39 | O60256 | 1 | 1 | 1 |
| 57 | MH | H | LSD1_HUMAN (O60341) Lysine-specific histone deme | 2.99E−04 | 92844.88 | O60341 | 1 | 1 | 1 |
| 58 | MH | L | FZD6_HUMAN (O60353) Frizzled 6 precursor (Frizzled | 1.13E−03 | 79222.06 | O60353 | 1 | 1 | 1 |
| 59 | MH | H | O60826 (O60826) JM1 protein (Hypothetical protein C [symbol] | 2.74E−03 | 70712.32 | O60826 | 1 | 1 | 1 |
| 60 | MH | G | GAS7_HUMAN (O60861) Growth-arrest-specific protei [symbol] | 3.84E−04 | 47236.75 | O60861 | 1 | 1 | 1 |
| 61 | MH | L | O75179 (O75179) KIAA0697 protein (Fragment) | 1.34E−03 | 263079.2 | O75179 | 1 | 1 | 1 |
| 62 | MH | F | PRPU_HUMAN (O94906) U5 snRNP-associated 102 k | 8.74E−03 | 106857.9 | O94906 | 1 | 1 | 1 |
| 63 | MH | F | SLIK3_HUMAN (O94933) SLIT and NTRK-like protein [symbol] | 9.29E−03 | 108937.1 | O94933 | 1 | 1 | 1 |
| 64 | MH | L | O95285 (O95285) Erythroblast macrophage protein E [symbol] | 8.16E−03 | 43879.31 | O95285 | 1 | 1 | 1 |
| 65 | MH | F | FMNL_HUMAN (O95466) Formin-like 1 protein (Formir | 9.19E−03 | 121750.9 | O95466 | 1 | 1 | 1 |
| 66 | MH | H | HPT_HUMAN (P00738) Haptoglobin precursor [Contai [symbol] | 2.71E−05 | 45176.59 | P00738 | 1 | 1 | 1 |
| 67 | MH | G | KV2B_HUMAN (P01615) Ig kappa chain V-II region FR | 5.81E−04 | 12651.75 | P01615 | 1 | 1 | 1 |
| 68 | MH | I | CO3A1_HUMAN (P02461) Collagen alpha 1(III) chain p | 4.25E−03 | 138470.2 | P02461 | 1 | 1 | 1 |
| 69 | MH | F | FIBG_HUMAN (P02679) Fibrinogen gamma chain pre [symbol] | 1.61E−04 | 51478.88 | P02679 | 1 | 1 | 1 |
| 70 | MH | H | CRP_HUMAN (P02741) C-reactive protein precursor [ [symbol] | 9.82E−03 | 25022.68 | P02741 | 1 | 1 | 1 |
| 71 | MH | I | AMBP_HUMAN (P02760) AMBP protein precursor [Co [symbol] | 1.37E−04 | 38973.99 | P02760 | 1 | 1 | 1 |
| 72 | MH | F | CSF1R_HUMAN (P07333) Macrophage colony-stimula | 8.66E−03 | 107915.2 | P07333 | 1 | 1 | 1 |
| 73 | MH | F | RET_HUMAN (P07949) Proto-oncogene tyrosine-prote | 4.41E−03 | 124239.4 | P07949 | 1 | 1 | 1 |
| 74 | MH | L | RARA_HUMAN (P10276) Retinoic acid receptor alpha [symbol] | 5.40E−04 | 50738.42 | P10276 | 1 | 1 | 1 |
| 75 | MH | H | C1TC_HUMAN (P11586) C-1-tetrahydrofolate synthas [symbol] | 2.60E−03 | 101364.5 | P11586 | 1 | 1 | 1 |
| 76 | MH | L | CSPG2_HUMAN (P13611) Versican core protein precu | 7.15E−06 | 372589 | P13611 | 1 | 1 | 1 |
| 77 | MH | L | K1C13_HUMAN (P13646) Keratin, type I cytoskeletal 1 | 4.22E−03 | 49555.45 | P13646 | 1 | 1 | 1 |
| 78 | MH | H | NEBU_HUMAN (P20929) Nebulin | 7.84E−04 | 772742.8 | P20929 | 1 | 1 | 1 |
| 79 | MH | F | PRDX2_HUMAN (P32119) Peroxiredoxin 2 (EC 1.11.1. | 9.46E−03 | 21747.2 | P32119 | 1 | 1 | 1 |
| 80 | MH | G | MYH11_HUMAN (P35749) Myosin-11 (Myosin heavy cl | 2.94E−03 | 227197.9 | P35749 | 1 | 1 | 1 |
| 81 | MH | G | BRCA1_HUMAN (P38398) Breast cancer type 1 susce [symbol] | 9.37E−03 | 207590.9 | P38398 | 1 | 1 | 1 |
| 82 | MH | L | RL13A_HUMAN (P40429) 60S ribosomal protein L13a | 9.48E−03 | 23431.35 | P40429 | 1 | 1 | 1 |
| 83 | MH | I | STAT1_HUMAN (P42224) Signal transducer and activa | 7.68E−03 | 87279.63 | P42224 | 1 | 1 | 1 |
| 84 | MH | H | RHG25_HUMAN (P42331) Rho-GTPase-activating pro | 8.14E−03 | 72385.19 | P42331 | 1 | 1 | 1 |
| 85 | MH | F | AFAM_HUMAN (P43652) Afamin precursor (Alpha-alb [symbol] | 8.12E−05 | 69024.09 | P43652 | 1 | 1 | 1 |
| 86 | MH | H | RBP2_HUMAN (P49792) Ran-binding protein 2 (RanB [symbol] | 3.16E−03 | 357991.4 | P49792 | 1 | 1 | 1 |
| 87 | MH | F | DPOG1_HUMAN (P54098) DNA polymerase gamma s | 3.35E−03 | 139473.4 | P54098 | 1 | 1 | 1 |
| 88 | MH | H | ELL_HUMAN (P55199) RNA polymerase II elongation f | 5.19E−03 | 68222.91 | P55199 | 1 | 1 | 1 |
| 89 | MH | G | CAD12_HUMAN (P55289) Brain-cadherin precursor (B | 1.52E−03 | 88219.76 | P55289 | 1 | 1 | 1 |
| 90 | MH | H | KR102_HUMAN (P60368) Keratin-associated protein 1 | 1.72E−03 | 25595.75 | P60368 | 1 | 1 | 1 |
| 91 | MH | L | RT21_HUMAN (P82921) Mitochondrial 28S ribosomal [symbol] | 2.34E−03 | 10734.48 | P82921 | 1 | 1 | 1 |
| 92 | MH | I | NSBP1_HUMAN (P82970) Nucleosomal binding protei [symbol] | 1.93E−03 | 31505.95 | P82970 | 1 | 1 | 1 |
| 93 | MH | G | SCN7A_HUMAN (Q01118) Sodium channel protein typ | 1.66E−03 | 193345 | Q01118 | 1 | 1 | 1 |
| 94 | MH | I | BDH_HUMAN (Q02338) D-beta-hydroxybutyrate dehyd | 5.68E−03 | 38132.47 | Q02338 | 1 | 1 | 1 |
| 95 | MH | L | CK013_HUMAN (Q02833) Protein C11orf13 (HRAS1-r [symbol] | 3.17E−03 | 39920.6 | Q02833 | 1 | 1 | 1 |
| 96 | MH | L | BPA1_HUMAN (Q03001) Bullous pemphigoid antigen 1 | 7.39E−03 | 371976.9 | Q03001 | 1 | 1 | 1 |
| 97 | MH | F | ATP7A_HUMAN (Q04656) Copper-transporting ATPas | 3.35E−03 | 163230 | Q04656 | 1 | 1 | 1 |
| 98 | MH | L | APLP2_HUMAN (Q06481) Amyloid-like protein 2 precu | 1.03E−09 | 86900.28 | Q06481 | 1 | 1 | 1 |
| 99 | MH | L | TRDN_HUMAN (Q13061) Triadin | 6.20E−03 | 81374.51 | Q13061 | 1 | 1 | 1 |
| 100 | MH | F | CSN1_HUMAN (Q13098) COP9 signalosome complex | 7.31E−03 | 53338.1 | Q13098 | 1 | 1 | 1 |
| 101 | MH | H | NOG2_HUMAN (Q13823) Nucleolar GTP-binding prote | 9.59E−03 | 83603.43 | Q13823 | 1 | 1 | 1 |
| 102 | MH | H | Q14159 (Q14159) KIAA0146 protein (Fragment) | 1.44E−03 | 100592.3 | Q14159 | 1 | 1 | 1 |
| 103 | MH | I | BMS1_HUMAN (Q14692) Ribosome biogenesis protei [symbol] | 3.76E−03 | 145715.5 | Q14692 | 1 | 1 | 1 |
| 104 | MH | L | 2A5D_HUMAN (Q14738) Serine/threonine protein phos | 2.98E−03 | 69947.5 | Q14738 | 1 | 1 | 1 |
| 105 | MH | F | RBBP5 HUMAN (Q15291) Retinoblastoma-binding pro | 9.99E−03 | 59044.69 | Q15291 | 1 | 1 | 1 |
| 106 | MH | F | CDC37_HUMAN (Q16543) Hsp90 co-chaperone Cdc3 [symbol] | 8.92E−03 | 44440.05 | Q16543 | 1 | 1 | 1 |
| 107 | MH | H | Q49AA0 (Q49AA0) ZNF642 protein | 5.50E−03 | 61112.29 | Q49AA0 | 1 | 1 | 1 |
| 108 | MH | G | Q4VC44 (Q4VC44) FLYWCH-type zinc finger 1, isoforr | 1.11E−03 | 79985.74 | Q4VC44 | 1 | 1 | 1 |
| 109 | MH | L | Q4VXM4 (Q4VXM4) Chromosome 20 open reading fra | 2.21E−03 | 114932.5 | Q4VXM4 | 1 | 1 | 1 |
| 110 | MH | F | Q53EU6 (Q53EU6) Hypothetical protein (Fragment) | 9.80E−04 | 48673.52 | Q53EU6 | 1 | 1 | 1 |
| 111 | MH | L | Q53RG2 (Q53RG2) Hypothetical protein CHRNG | 4.34E−03 | 58259.47 | Q53RG2 | 1 | 1 | 1 |
| 112 | MH | L | Q562E5 (Q562E5) KIAA0194 protein (Fragment) | 3.73E−04 | 157553.1 | Q562E5 | 1 | 1 | 1 |
| 113 | MH | G | Q59FI9 (Q59FI9) Ribosomal protein L12 variant (Frag [symbol] | 6.72E−03 | 21469.49 | Q59FI9 | 1 | 1 | 1 |
| 114 | MH | H | Q5JPU1 (Q5JPU1) Pyruvate dehydrogenase (Lipoamid | 3.07E−03 | 22743.74 | Q5JPU1 | 1 | 1 | 1 |
| 115 | MH | L | Q5JTH6 (Q5JTH6) Exosomal core protein CSL4 | 5.99E−04 | 18804.54 | Q5JTH6 | 1 | 1 | 1 |
| 116 | MH | L | Q5JX69 (Q5JX69) OTTHUMP00000031352 | 8.08E−03 | 19485.96 | Q5JX69 | 1 | 1 | 1 |
| 117 | MH | G | Q5T1J6 (Q5T1J6) OTTHUMP00000030508 | 4.35E−03 | 17161.46 | Q5T1J6 | 1 | 1 | 1 |
| 118 | MH | H | Q5TDG2 (Q5TDG2) Hydroxy-delta-5-steroid dehydrog [symbol] | 2.32E−03 | 42427.11 | Q5TDG2 | 1 | 1 | 1 |
| 119 | MH | L | Q5TYW1 (Q5TYW1) Novel protein (Fragment) | 4.42E−03 | 97254.69 | Q5TYW1 | 1 | 1 | 1 |
| 120 | MH | G | Q5VXI2 (Q5VXI2) Myosin IIIA | 7.99E−03 | 22191.69 | Q5VXI2 | 1 | 1 | 1 |
| 121 | MH | H | Q5VYM8 (Q5VYM8) Unc-13 homolog B (C. elegans) | 2.31E−03 | 180563.2 | Q5VYM8 | 1 | 1 | 1 |
| 122 | MH | I | Q5XPV5 (Q5XPV5) Sjogren syndrome antigen A1 | 5.68E−04 | 54089.2 | Q5XPV5 | 1 | 1 | 1 |
| 123 | MH | F | Q63HP7 (Q63HP7) Hypothetical protein DKFZp686P15 | 5.39E−03 | 65621.97 | Q63HP7 | 1 | 1 | 1 |
| 124 | MH | H | Q659F3 (Q659F3) Hypothetical protein DKFZp434J194 | 4.86E−03 | 14956.27 | Q659F3 | 1 | 1 | 1 |
| 125 | MH | F | CEP4_HUMAN (Q66GS9) Centrosomal protein 4 (Cen [symbol] | 9.02E−04 | 133421.9 | Q66GS9 | 1 | 1 | 1 |
| 126 | MH | I | Q68CI8 (Q68CI8) Hypothetical protein HMFT1272 (Fra [symbol] | 2.38E−03 | 48598.88 | Q68CI8 | 1 | 1 | 1 |
| 127 | MH | L | Q68DL8 (Q68DL8) Hypothetical protein DKFZp781L03 [symbol] | 3.98E−03 | 84645.59 | Q68DL8 | 1 | 1 | 1 |
| 128 | MH | F | S6A19_HUMAN (Q695T7) Sodium-dependent neutral [symbol] | 5.44E−03 | 71063.29 | Q695T7 | 1 | 1 | 1 |
| 129 | MH | L | Q6A334 (Q6A334) Mutated in bladder cancer 1 | 8.74E−03 | 61882.69 | Q6A334 | 1 | 1 | 1 |
| 130 | MH | F | Q6IA31 (Q6IA31) FLJ14154 protein | 7.48E−03 | 27415.97 | Q6IA31 | 1 | 1 | 1 |
| 131 | MH | G | Q6IBW4 (Q6IBW4) Em: U62317.2 protein | 6.70E−03 | 68183.7 | Q6IBW4 | 1 | 1 | 1 |
| 132 | MH | I | Q6IE81 (Q6IE81) JADE1L protein | 9.07E−03 | 95472.82 | Q6IE81 | 1 | 1 | 1 |
| 133 | MH | I | Q6IF05 (Q6IF05) Olfactory receptor OR1-47 | 8.84E−04 | 37737.82 | Q6IF05 | 1 | 1 | 1 |
| 134 | MH | I | ST6B1_HUMAN (Q6IMI4) Sulfotransferase 6B1 (EC 2 [symbol] | 9.60E−03 | 30491.21 | Q6IMI4 | 1 | 1 | 1 |
| 135 | MH | G | PTRF_HUMAN (Q6NZI2) Polymerase I and transcript [symbol] | 1.14E−03 | 43449.88 | Q6NZI2 | 1 | 1 | 1 |
| 136 | MH | I | Q6P3W7 (Q6P3W7) Similar to mouse D10Ertd802e pr [symbol] | 9.06E−04 | 103642.4 | Q6P3W7 | 1 | 1 | 1 |
| 137 | MH | F | Q6P658 (Q6P658) FNBP1 protein (Fragment) | 6.14E−04 | 40759.38 | Q6P658 | 1 | 1 | 1 |
| 138 | MH | H | GP133_HUMAN (Q6QNK2) Probable G-protein couple [symbol] | 7.32E−03 | 96468.2 | Q6QNK2 | 1 | 1 | 1 |
| 139 | MH | F | Q6UXX5 (Q6UXX5) ITI-like protein (Inter-alpha (Globul | 6.30E−03 | 143097.8 | Q6UXX5 | 1 | 1 | 1 |
| 140 | MH | H | Q6ZMV7 (Q6ZMV7) Hypothetical protein FLJ16641 | 5.36E−03 | 45118.72 | Q6ZMV7 | 1 | 1 | 1 |
| 141 | MH | L | Q6ZMX7 (Q6ZMX7) Hypothetical protein FLJ16607 | 1.23E−03 | 147809.7 | Q6ZMX7 | 1 | 1 | 1 |
| 142 | MH | L | Q6ZQN2 (Q6ZQN2) Hypothetical protein FLJ46846 | 2.18E−03 | 180573.9 | Q6ZQN2 | 1 | 1 | 1 |
| 143 | MH | H | Q6ZVL2 (Q6ZVL2) Hypothetical protein FLJ42427 | 7.92E−03 | 15702.14 | Q6ZVL2 | 1 | 1 | 1 |
| 144 | MH | F | Q6ZW87 (Q6ZW87) Hypothetical protein FLJ41443 | 5.09E−03 | 24773.63 | Q6ZW87 | 1 | 1 | 1 |
| 145 | MH | I | Q7KYS7 (Q7KYS7) Hypothetical protein (Fragment) | 1.53E−03 | 31128.16 | Q7KYS7 | 1 | 1 | 1 |
| 146 | MH | G | Q7RTS7 (Q7RTS7) Keratin 5c | 4.83E−03 | 59333.45 | Q7RTS7 | 1 | 1 | 1 |
| 147 | MH | F | CA036_HUMAN (Q7Z3Z2) Protein C1orf36 | 1.27E−04 | 22689.57 | Q7Z3Z2 | 1 | 1 | 1 |
| 148 | MH | I | Q7Z736 (Q7Z736) Hypothetical protein FLJ21019 | 7.52E−03 | 85273.38 | Q7Z736 | 1 | 1 | 1 |
| 149 | MH | I | Q86VJ1 (Q86VJ1) E3 ligase for inhibin receptor | 9.51E−03 | 289427.4 | Q86VJ1 | 1 | 1 | 1 |
| 150 | MH | L | Q86Y22 (Q86Y22) Alpha 1 type XXIII collagen | 3.71E−03 | 51912.29 | Q86Y22 | 1 | 1 | 1 |
| 151 | MH | F | Q8IUA7 (Q8IUA7) ATP-binding cassette sub-family An | 5.35E−03 | 184241.6 | Q8IUA7 | 1 | 1 | 1 |
| 152 | MH | G | Q8IUG8 (Q8IUG8) Carnitine transporter 2 | 6.69E−03 | 60830.3 | Q8IUG8 | 1 | 1 | 1 |
| 153 | MH | F | Q8IUS2 (Q8IUS2) LOC339483 protein | 3.48E−03 | 2870.257 | Q8IUS2 | 1 | 1 | 1 |
| 154 | MH | L | Q8IZA4 (Q8IZA4) ELYS transcription factor-like protein | 5.66E−03 | 256022.6 | Q8IZA4 | 1 | 1 | 1 |
| 155 | MH | L | ASPM_HUMAN (Q8IZT6) Abnormal spindle-like microc | 4.72E−03 | 409539.8 | Q8IZT6 | 1 | 1 | 1 |
| 156 | MH | G | Q8N1G4 (Q8N1G4) Hypothetical protein LRRC47 (Nov | 2.19E−03 | 63433.89 | Q8N1G4 | 1 | 1 | 1 |
| 157 | MH | H | Q8N401 (Q8N401) KIAA1632 protein | 8.67E−03 | 52340.19 | Q8N401 | 1 | 1 | 1 |
| 158 | MH | L | DCAK2_HUMAN (Q8N568) Serine/threonine-protein ki [symbol] | 4.26E−03 | 83587.57 | Q8N568 | 1 | 1 | 1 |
| 159 | MH | L | Q8N5E0 (Q8N5E0) Apoptosis-inducing factor like, isof [symbol] | 5.12E−03 | 65924.3 | Q8N5E0 | 1 | 1 | 1 |
| 160 | MH | F | Q8NC99 (Q8NC99) Hypothetical protein FLJ90396 | 5.35E−03 | 66758.55 | Q8NC99 | 1 | 1 | 1 |
| 161 | MH | H | Q8NF06 (Q8NF06) FLJ00398 protein (Fragment) | 1.78E−04 | 72000.41 | Q8NF06 | 1 | 1 | 1 |
| 162 | MH | G | Q8NFY8 (Q8NFY8) Neuroblastoma-amplified protein | 1.13E−03 | 268295.4 | Q8NFY8 | 1 | 1 | 1 |
| 163 | MH | L | O10A7_HUMAN (Q8NGE5) Olfactory receptor 10A7 | 8.56E−03 | 35670.34 | Q8NGE5 | 1 | 1 | 1 |
| 164 | MH | F | Q8NH29 (Q8NH29) Seven transmembrane helix recep [symbol] | 1.70E−04 | 25373.85 | Q8NH29 | 1 | 1 | 1 |
| 165 | MH | G | IL17D_HUMAN (Q8TAD2) Interleukin-17D precursor (II | 6.37E−03 | 21879.14 | Q8TAD2 | 1 | 1 | 1 |
| 166 | MH | F | Q8TCE1 (Q8TCE1) SERPINC1 protein | 2.30E−05 | 29073.79 | Q8TCE1 | 1 | 1 | 1 |
| 167 | MH | F | Q8TCJ2 (Q8TCJ2) Source of immunodominant MHC-a | 3.14E−04 | 93613.76 | Q8TCJ2 | 1 | 1 | 1 |
| 168 | MH | F | DDX54_HUMAN (Q8TDD1) DEAD-box protein 54 (EC | 1.74E−03 | 98534.24 | Q8TDD1 | 1 | 1 | 1 |
| 169 | MH | G | Q92772 (Q92772) P56 KKIAMRE protein kinase (Cycli [symbol] | 6.77E−03 | 55982.93 | Q92772 | 1 | 1 | 1 |
| 170 | MH | L | COR2A_HUMAN (Q92828) Coronin-2A (WD repeat-co | 3.04E−03 | 59725.43 | Q92828 | 1 | 1 | 1 |
| 171 | MH | G | Q96E20 (Q96E20) CD99L2 protein (Hypothetical protei | 3.63E−03 | 16612.17 | Q96E20 | 1 | 1 | 1 |
| 172 | MH | L | Q96IV6 (Q96IV6) C5orf4 protein | 4.48E−03 | 38976.07 | Q96IV6 | 1 | 1 | 1 |
| 173 | MH | L | RSRC1_HUMAN (Q96IZ7) Arginine/serine-rich coiled c | 8.17E−03 | 38654.46 | Q96IZ7 | 1 | 1 | 1 |
| 174 | MH | G | KCNH8_HUMAN (Q96L42) Potassium voltage-gated c [symbol] | 3.90E−04 | 123754.3 | Q96L42 | 1 | 1 | 1 |
| 175 | MH | F | Q96MP4 (Q96MP4) Hypothetical protein FLJ32091 | 6.67E−03 | 81413.94 | Q96MP4 | 1 | 1 | 1 |
| 176 | MH | H | Q96NI8 (Q96NI8) Hypothetical protein FLJ30791 | 4.72E−03 | 62289.69 | Q96NI8 | 1 | 1 | 1 |
| 177 | MH | H | ZN285_HUMAN (Q96NJ3) Zinc finger protein 285 | 2.66E−03 | 50009.57 | Q96NJ3 | 1 | 1 | 1 |
| 178 | MH | H | INP4A_HUMAN (Q96PE3) Type I inositol-3,4-bisphosp [symbol] | 2.98E−04 | 109885.5 | Q96PE3 | 1 | 1 | 1 |
| 179 | MH | G | UHRF2_HUMAN (Q96PU4) Ubiquitin-like containing P [symbol] | 6.47E−03 | 89927.86 | Q96PU4 | 1 | 1 | 1 |
| 180 | MH | G | Q96QE0 (Q96QE0) Hypothetical protein DL8Q12 | 3.39E−03 | 23959.2 | Q96QE0 | 1 | 1 | 1 |
| 181 | MH | H | PANX2_HUMAN (Q96RD6) Pannexin-2 | 7.29E−03 | 69434.98 | Q96RD6 | 1 | 1 | 1 |
| 182 | MH | G | Q96RF2 (Q96RF2) WWOXdelta5-8 | 3.55E−03 | 35019.5 | Q96RF2 | 1 | 1 | 1 |
| 183 | MH | L | LYST_HUMAN (Q99698) Lysosomal trafficking regulat [symbol] | 7.47E−03 | 428865.6 | Q99698 | 1 | 1 | 1 |
| 184 | MH | H | SMO_HUMAN (Q99835) Smoothened homolog precur [symbol] | 7.04E−05 | 86341.18 | Q99835 | 1 | 1 | 1 |
| 185 | MH | L | Q99993 (Q99993) WUGSC:H_2G3A.1 protein | 4.36E−03 | 60051.64 | Q99993 | 1 | 1 | 1 |
| 186 | MH | L | OSR10_HUMAN (Q9BXB5) Oxysterol binding protein-r [symbol] | 7.10E−03 | 83917.38 | Q9BXB5 | 1 | 1 | 1 |
| 187 | MH | H | EMR3_HUMAN (Q9BY15) EGF-like module containing | 6.97E−03 | 72545.69 | Q9BY15 | 1 | 1 | 1 |
| 188 | MH | F | Q9BYJ0 (Q9BYJ0) Ksp37 (HBp17-related protein) | 6.42E−04 | 24565.15 | Q9BYJ0 | 1 | 1 | 1 |
| 189 | MH | G | OSR7_HUMAN (Q9BZF2) Oxysterol binding protein-rel | 3.33E−04 | 95371.7 | Q9BZF2 | 1 | 1 | 1 |
| 190 | MH | L | Q9BZH2 (Q9BZH2) False p73 target protein | 2.66E−03 | 53043.29 | Q9BZH2 | 1 | 1 | 1 |
| 191 | MH | I | PHAR1_HUMAN (Q9C0D0) Phosphatase and actin reg | 2.72E−04 | 54573.55 | Q9C0D0 | 1 | 1 | 1 |
| 192 | MH | G | Q9C0D4 (Q9C0D4) KIAA1729 protein (Fragment) | 1.84E−03 | 113646.6 | Q9C0D4 | 1 | 1 | 1 |
| 193 | MH | L | DDX24_HUMAN (Q9GZR7) ATP-dependent RNA helic | 7.26E−03 | 96271.52 | Q9GZR7 | 1 | 1 | 1 |
| 194 | MH | L | Q9GZT6 (Q9GZT6) MDS011 (Hypothetical protein MD [symbol] | 4.53E−03 | 29453.17 | Q9GZT6 | 1 | 1 | 1 |
| 195 | MH | H | Q9H0J7 (Q9H0J7) Hypothetical protein DKFZp434F01 | 2.81E−04 | 53323.07 | Q9H0J7 | 1 | 1 | 1 |
| 196 | MH | L | GHITM_HUMAN (Q9H3K2) Growth hormone inducible | 4.28E−03 | 37180.59 | Q9H3K2 | 1 | 1 | 1 |
| 197 | MH | H | Q9H3M9 (Q9H3M9) Homo sapiens (Fragment) | 5.36E−03 | 39581.41 | Q9H3M9 | 1 | 1 | 1 |
| 198 | MH | H | RANB3_HUMAN (Q9H6Z4) Ran-binding protein 3 (Ran | 3.82E−03 | 60173.32 | Q9H6Z4 | 1 | 1 | 1 |
| 199 | MH | H | SMYD3_HUMAN (Q9H7B4) SET and MYND domain-c [symbol] | 5.56E−03 | 49050.59 | Q9H7B4 | 1 | 1 | 1 |
| 200 | MH | L | Q9H7C4 (Q9H7C4) Hypothetical protein FLJ21054 | 8.68E−03 | 17715.02 | Q9H7C4 | 1 | 1 | 1 |
| 201 | MH | L | COG4_HUMAN (Q9H9E3) Conserved oligomeric Golgi | 9.55E−03 | 89038.48 | Q9H9E3 | 1 | 1 | 1 |
| 202 | MH | G | UBP29_HUMAN (Q9HBJ7) Ubiquitin carboxyl-terminal | 1.22E−03 | 104090.1 | Q9HBJ7 | 1 | 1 | 1 |
| 203 | MH | H | Q9HBY0 (Q9HBY0) Putative superoxide-generating NA | 4.96E−03 | 64893.13 | Q9HBY0 | 1 | 1 | 1 |
| 204 | MH | F | SYTL2_HUMAN (Q9HCH5) Synaptotagmin-like protein | 8.61E−04 | 100734.4 | Q9HCH5 | 1 | 1 | 1 |
| 205 | MH | F | Q9NR16 (Q9NR16) Scavenger receptor cysteine-rich t [symbol] | 6.17E−03 | 159159.5 | Q9NR16 | 1 | 1 | 1 |
| 206 | MH | L | SPTN5_HUMAN (Q9NRC6) Spectrin beta chain, brain | 3.96E−04 | 416577.2 | Q9NRC6 | 1 | 1 | 1 |
| 207 | MH | L | DC13_HUMAN (Q9NRP2) UPF0287 protein DC13 | 2.81E−03 | 9453.72 | Q9NRP2 | 1 | 1 | 1 |
| 208 | MH | F | Q9NS87 (Q9NS87) Kinesin-like protein 2 | 4.45E−03 | 160060.4 | Q9NS87 | 1 | 1 | 1 |
| 209 | MH | G | Q9NSN6 (Q9NSN6) Hypothetical protein DKFZp761O0 | 6.56E−03 | 39970.1 | Q9NSN6 | 1 | 1 | 1 |
| 210 | MH | H | EMIL3_HUMAN (Q9NT22) EMILIN-3 precursor (EMILIN | 3.69E−03 | 82595.73 | Q9NT22 | 1 | 1 | 1 |
| 211 | MH | H | UGGG1_HUMAN (Q9NYU2) UDP-glucose: glycoprotei [symbol] | 3.83E−03 | 174866.5 | Q9NYU2 | 1 | 1 | 1 |
| 212 | MH | H | T2R14_HUMAN (Q9NYV8) Taste receptor type 2 mem | 1.33E−03 | 36136.48 | Q9NYV8 | 1 | 1 | 1 |
| 213 | MH | F | COMD9_HUMAN (Q9P000) COMM domain containing | 4.29E−04 | 21904.53 | Q9P000 | 1 | 1 | 1 |
| 214 | MH | L | VPS18_HUMAN (Q9P253) Vacuolar protein sorting 18 | 7.01E−03 | 110115.9 | Q9P253 | 1 | 1 | 1 |
| 215 | MH | I | RRBP1_HUMAN (Q9P2E9) Ribosome-binding protein | 4.24E−03 | 152380 | Q9P2E9 | 1 | 1 | 1 |
| 216 | MH | G | SUCB1_HUMAN (Q9P2R7) Succinyl-CoA ligase [ADP- | 8.47E−03 | 50299.33 | Q9P2R7 | 1 | 1 | 1 |
| 217 | MH | I | GT2D1_HUMAN (Q9UHL9) General transcription facto [symbol] | 4.56E−03 | 105990.8 | Q9UHL9 | 1 | 1 | 1 |
| 218 | MH | G | Q9UI74 (Q9UI74) PRO0245 | 7.12E−04 | 8075.004 | Q9UI74 | 1 | 1 | 1 |
| 219 | MH | L | SPB13_HUMAN (Q9UIV8) Serpin B13 (Hurpin) (HaCa [symbol] | 8.72E−04 | 44248.2 | Q9UIV8 | 1 | 1 | 1 |
| 220 | MH | I | HOOK1_HUMAN (Q9UJC3) Hook homolog 1 (h-hook1 | 9.77E−03 | 84594.84 | Q9UJC3 | 1 | 1 | 1 |
| 221 | MH | F | DKKL1_HUMAN (Q9UK85) Dickkopf-like protein 1 pre [symbol] | 1.36E−04 | 26990.23 | Q9UK85 | 1 | 1 | 1 |
| 222 | MH | L | PADI3_HUMAN (Q9ULW8) Protein-arginine deiminase | 9.91E−04 | 74695.37 | Q9ULW8 | 1 | 1 | 1 |
| 223 | MH | H | MLL4_HUMAN (Q9UMN6) Myeloid/lymphoid or mixed-I | 9.43E−03 | 293327.3 | Q9UMN6 | 1 | 1 | 1 |
| 224 | MH | H | GPR34_HUMAN (Q9UPC5) Probable G-protein couple | 8.71E−03 | 43831.09 | Q9UPC5 | 1 | 1 | 1 |
| 225 | MH | G | Q9UQ10 (Q9UQ10) Dimeric dihydrodiol dehydrogenas [symbol] | 9.73E−03 | 36358.77 | Q9UQ10 | 1 | 1 | 1 |
| 226 | MH | I | SHOC2_HUMAN (Q9UQ13) Leucine-rich repeat protei [symbol] | 3.26E−03 | 64847.64 | Q9UQ13 | 1 | 1 | 1 |
| 227 | MH | G | AT11B_HUMAN (Q9Y2G3) Probable phospholipid-tran [symbol] | 9.84E−03 | 134104.1 | Q9Y2G3 | 1 | 1 | 1 |
| 228 | MH | F | PCDG7_HUMAN (Q9Y5G6) Protocadherin gamma A7 | 2.96E−03 | 101659.2 | Q9Y5G6 | 1 | 1 | 1 |
| 229 | MH | L | PCDA2_HUMAN (Q9Y5H9) Protocadherin alpha 2 pre [symbol] | 6.26E−03 | 102000.4 | Q9Y5H9 | 1 | 1 | 1 |
| 230 | MH | G | Q9Y5S5 (Q9Y5S5) DNA polymerase epsilon catalytic s | 9.10E−03 | 262846.1 | Q9Y5S5 | 1 | 1 | 1 |
| 231 | MH | F | SO1B1_HUMAN (Q9Y6L6) Solute carrier organic anion | 1.51E−03 | 76398.98 | Q9Y6L6 | 1 | 1 | 1 |

| | Sub-cellular | |
|---|---|---|
| Protein | Location | Function |
| Sortilin-related receptor precursor | membrane | endocytic receptor |
| Neuronal membrane glycoprotein M6-a | membrane | neural development |
| Kinesin-like protein KIF1A | synaptic vesicles | axonal transport |
| Voltage-dependent T-type calcium channel alpha-1G subunit | membrane | calcium influx/eflux |
| Ryanodine receptor 2 (Cardiac muscle-type ryanodine receptor) | membrane | Communication between transverse- |
| | | tubules and sarcoplasmic reticulum |
| SOX-12 protein (SOX-22 protein) | nucleus | non-histone DNA binding |
| Frizzled 6 precursor (Frizzled-6) (Fz-6) (hFz6) | membrane | cell differentitation/morphogenesis |
| Retinoblastoma-binding protein 5 (RBBP-5) | nucleus | transcription |
| Retinoic acid receptor alpha (RAR-alpha) | nucleus | retonic acid receptor |

8.0 ul vitreous/patient
8.0 × 5 = 40 ul/patient for study

TABLE 9
Vitreous proteins identified from patients with retinal detachment (RD)

| | Protein | Accession | MW | P (pro) |
|---|---|---|---|---|
| 1 | LOC131076 protein | Q4VC31 | 16609.49 | 2.50E−03 |
| 2 | Keratin, type II | Q14533 | 54935.82 | 5.27E−07 |
| 3 | Histone acetyltransferase | Q92831 | 92953.77 | 6.68E−04 |
| 4 | Cathepsin D precursor | P07339 | 44523.66 | 3.66E−09 |
| 5 | Peroxiredoxin 1 | Q06830 | 22096.28 | 1.57E−06 |
| 6 | Nuclear pore complex protein Nup214 | P35658 | 213632.8 | 4.38E−03 |
| 7 | T-cell activation Rho GTPase-activating protein | Q8N103 | 80652.9 | 5.60E−03 |
| 8 | Protein C9orf126 | Q8N9R8 | 70385.07 | 1.82E−03 |
| 9 | Receptor interacting protein kinase 5 | Q5RKT0 | 99957.35 | 2.01E−03 |
| 10 | Novel protein | Q5VVM6 | 91277.53 | 2.74E−04 |
| 11 | Potassium voltage-gated channel subfamily A | Q09470 | 56505.6 | 8.04E−04 |
| 12 | Huntingtin interacting protein E | Q9BVA6 | 51745.16 | 7.33E−05 |
| 13 | KIAA0068 protein (Fragment) | Q14467 | 147040.6 | 2.65E−04 |
| 14 | Hypothetical protein FLJ21129 | Q9H796 | 63276.44 | 2.87E−03 |
| 15 | HS1-associating protein X-1 | O00165 | 31601.06 | 2.63E−03 |
| 16 | Inositol polyphosphate 5-phosphatase | O15357 | 138498.9 | 1.69E−04 |
| 17 | DNA repair protein RAD51 homolog 3 | O43502 | 42162.89 | 3.18E−03 |
| 18 | Ig lambda chain V region | P04211 | 12372.02 | 4.35E−03 |
| 19 | Keratin, type II | P04259 | 59831.25 | 4.19E−05 |
| 20 | Cation-independent mannose-6-phosphate receptor | P11717 | 274097.6 | 5.66E−03 |
| 21 | Macrophage scavenger receptor types I and II | P21757 | 49730.82 | 7.76E−04 |
| 22 | Matrin-3 | P43243 | 94564.67 | 3.80E−03 |
| 23 | Voltage-dependent anion-selective channel protein 2 | P45880 | 38068.68 | 4.14E−04 |
| 24 | FLJ00268 protein (Fragment) | Q6ZML5 | 72805.3 | 2.78E−03 |
| 25 | LAG1 longevity assurance homolog 2 | Q96G23 | 44847.38 | 1.46E−04 |
| 26 | Serine/threonine-protein kinase | Q96J92 | 134655.6 | 8.12E−03 |
| 27 | Hypothetical protein FLJ13940 | Q9H853 | 27528.79 | 2.89E−03 |
| 28 | Adenylate kinase isoenzyme 2 | P54819 | 26329.73 | 1.02E−03 |
| 29 | DAX1_HUMAN (P51843) Nuclear receptor 0B1 (Nuclear receptor DAX-1) (DSS-AHC | P51843 | 51683.89 | 1.40E−03 |
| 30 | ZN473_HUMAN (Q8WTR7) Zinc finger protein 473 (Zinc finger protein 100 homolog) | Q8WTR7 | 100118.1 | 2.47E−03 |
| 31 | CPSF3_HUMAN (Q9UKF6) Cleavage and polyadenylation specificity factor, 73 kDa su | Q9UKF6 | 77436.22 | 1.14E−03 |
| 32 | K2C5_HUMAN (P13647) Keratin, type II cytoskeletal 5 (Cytokeratin-5) (CK-5) (Ke | P13647 | 62409.08 | 7.34E−08 |
| 33 | CAH1_HUMAN (P00915) Carbonic anhydrase 1 (EC 4.2.1.1) (Carbonic anhydrase I) ( | P00915 | 28721.34 | 8.52E−08 |
| 34 | KNG1_HUMAN (P01042) Kininogen-1 precursor (Alpha-2-thiol proteinase inhibitor) | P01042 | 71900.1 | 2.12E−10 |
| 35 | K1C14_HUMAN (P02533) Keratin, type I cytoskeletal 14 (Cytokeratin-14) (CK-14) | P02533 | 51458.45 | 3.33E−06 |
| 36 | O00211 (O00211) HSLK (Ste20-related serineVthreonine kinase) | O00211 | 138909.7 | 8.39E−03 |
| 37 | P3C2A_HUMAN (O00443) Phosphatidylinositol-4-phosphate 3-kinase C2 domain-cont | O00443 | 190616.2 | 7.07E−03 |
| 38 | SYT5_HUMAN (O00445) Synaptotagmin-5 (Synaptotagmin V) (SytV) | O00445 | 42873.54 | 3.71E−03 |
| 39 | CHD2_HUMAN (O14647) Chromodomain-helicase-DNA-binding protein 2 (CHD-2) | O14647 | 200437.6 | 7.04E−03 |
| 40 | MLL2_HUMAN (O14686) Myeloid/lymphoid or mixed-lineage leukemia protein 2 (ALL1 | O14686 | 563835.8 | 8.21E−04 |
| 41 | O15042 (O15042) Hypothetical protein KIAA0332 (U2-associated SR140 protein) (Fr | O15042 | 118087.4 | 7.16E−04 |
| 42 | SNPH_HUMAN (O15079) Syntaphilin | O15079 | 57953.73 | 7.58E−03 |
| 43 | WDR46_HUMAN (O15213) WD-repeat protein 46 (WD-repeat protein BING4) | O15213 | 67998.89 | 3.67E−03 |
| 44 | HAPIP_HUMAN (O60229) Huntingtin-associated protein-interacting protein (Duo pr | O60229 | 192107.9 | 3.37E−03 |
| 45 | O60687 (O60687) Sushi-repeat-containing protein, X-linked 2 | O60687 | 52937.79 | 4.07E−03 |
| 46 | ZC11A_HUMAN (O75152) Zinc finger CCCH-type domain containing protein 11A | O75152 | 89076.06 | 4.33E−03 |
| 47 | O75198 (O75198) CDC37-like gene | O75198 | 26530.93 | 1.49E−03 |
| 48 | AVIL_HUMAN (O75366) Advillin (p92) | O75366 | 92028.35 | 8.01E−03 |
| 49 | MYCB2_HUMAN (O75592) Probable ubiquitin ligase protein MYCBP2 (EC 6.3.2.—) (My | O75592 | 509842.3 | 7.36E−03 |
| 50 | CMC1_HUMAN (O75746) Calcium-binding mitochondrial carrier protein Aralar1 (Mit | O75746 | 74709.01 | 3.32E−03 |
| 51 | MFN2_HUMAN (O95140) Transmembrane GTPase MFN2 (EC 3.6.5.—) (Mitofusin-2) | O95140 | 86346.95 | 4.18E−03 |
| 52 | G6PE_HUMAN (O95479) GDH/6PGL endoplasmic bifunctional protein precursor [Incl | O95479 | 88822.63 | 3.69E−03 |
| 53 | HPTR_HUMAN (P00739) Haptoglobin-related protein precursor | P00739 | 38982.66 | 5.05E−03 |
| 54 | CFAB_HUMAN (P00751) Complement factor B precursor (EC 3.4.21.47) (C3/C5 conv | P00751 | 85478.58 | 2.24E−04 |
| 55 | ASSY_HUMAN (P00966) Argininosuccinate synthase (EC 6.3.4.5) (Citrulline-aspar | P00966 | 46501.01 | 4.02E−03 |
| 56 | HRG_HUMAN (P04196) Histidine-rich glycoprotein precursor (Histidine-proline-ri | P04196 | 59540.94 | 5.94E−04 |
| 57 | CFAI_HUMAN (P05156) Complement factor I precursor (EC 3.4.21.45) (C3B/C4B inac | P05156 | 65676.66 | 2.93E−05 |
| 58 | ITB1_HUMAN (P05556) Integrin beta-1 precursor (Fibronectin receptor beta subun | P05556 | 88406.98 | 7.50E−05 |
| 59 | KV2F_HUMAN (P06310) Ig kappa chain V-II region RPMI 6410 precursor | P06310 | 14697.37 | 1.78E−06 |
| 60 | K1C16_HUMAN (P08779) Keratin, type I cytoskeletal 16 (Cytokeratin-16) (CK-16) ( | P08779 | 51105.21 | 1.08E−06 |
| 61 | TAU_HUMAN (P10636) Microtubule-associated protein tau (Neurofibrillary tangle | P10636 | 78698.83 | 7.47E−04 |
| 62 | PABP1_HUMAN (P11940) Polyadenylate-binding protein 1 (Poly(A)-binding protein | P11940 | 70626.04 | 9.99E−03 |
| 63 | SCG2_HUMAN (P13521) Secretogranin-2 precursor (Secretogranin II) (SgII) (Chromo | P13521 | 70825.11 | 6.93E−03 |
| 64 | UCHL3_HUMAN (P15374) Ubiquitin carboxyl-terminal hydrolase isozyme L3 (EC 3.4. | P15374 | 26165.98 | 6.36E−03 |
| 65 | CBPE_HUMAN (P16870) Carboxypeptidase E precursor (EC 3.4.17.10) (CPE) (Carbo: | P16870 | 53117.17 | 1.65E−06 |
| 66 | AATC_HUMAN (P17174) Aspartate aminotransferase, cytoplasmic (EC 2.6.1.1) (Tran | P17174 | 46087.54 | 9.28E−03 |
| 67 | A1ATR_HUMAN (P20848) Alpha-1-antitrypsin-related protein precursor | P20848 | 47860.97 | 7.19E−03 |
| 68 | RAE2_HUMAN (P26374) Rab proteins geranylgeranyltransferase component A 2 (Rab | P26374 | 74024.21 | 1.83E−03 |
| 69 | VGFR2_HUMAN (P35968) Vascular endothelial growth factor receptor 2 precursor ( | P35968 | 151430.5 | 1.56E−03 |
| 70 | CH3L1_HUMAN (P36222) Chitinase-3-like protein 1 precursor (Cartilage glycoprot | P36222 | 42586.39 | 2.71E−06 |
| 71 | MAP1B_HUMAN (P46821) Microtubule-associated protein 1B (MAP 1B) [Contains: M | P46821 | 270451.8 | 5.22E−03 |
| 72 | PCY1A_HUMAN (P49585) Choline-phosphate cytidylyltransferase A (EC 2.7.7.15) (P | P49585 | 41705.8 | 3.65E−03 |
| 73 | CEBPD_HUMAN (P49716) CCAAT/enhancer binding protein delta (C/EBP delta) (Nuc | P49716 | 28461.45 | 6.61E−03 |
| 74 | CCR6_HUMAN (P51684) C—C chemokine receptor type 6 (C—C CKR-6) (CC-CKR-6) ( | P51684 | 42465.93 | 7.26E−03 |
| 75 | EPHA5_HUMAN (P54756) Ephrin type-A receptor 5 precursor (EC 2.7.1.112) (Tyrosi | P54756 | 114710.3 | 7.74E−03 |
| 76 | PSD4_HUMAN (P55036) 26S proteasome non-ATPase regulatory subunit 4 (26S pro | P55036 | 40711.21 | 6.62E−04 |
| 77 | B2MG_HUMAN (P61769) Beta-2-microglobulin precursor [Contains: Beta-2-microglob | P61769 | 13705.91 | 3.51E−07 |
| 78 | GBB1_HUMAN (P62873) Guanine nucleotide-binding protein G(I)/G(S)/G(T) beta sub | P62873 | 37221.98 | 1.27E−03 |
| 79 | CXAR_HUMAN (P78310) Coxsackievirus and adenovirus receptor precursor (Coxsac | P78310 | 40004.39 | 1.03E−03 |
| 80 | GALT1_HUMAN (Q10472) Polypeptide N-acetylgalactosaminyltransferase 1 (EC 2.4.1 | Q10472 | 64177.48 | 3.87E−03 |
| 81 | PTN13_HUMAN (Q12923) Tyrosine-protein phosphatase, non-receptor type 13 (EC 3. | Q12923 | 276731.3 | 3.13E−05 |
| 82 | DLG1_HUMAN (Q12959) Presynaptic protein SAP97 (Synapse-associated protein 97) | Q12959 | 100293.1 | 6.56E−03 |
| 83 | AKAP6_HUMAN (Q13023) A-kinase anchor protein 6 (Protein kinase A anchoring pro | Q13023 | 256503.6 | 1.63E−03 |
| 84 | ATM_HUMAN (Q13315) Serine-protein kinase ATM (EC 2.7.1.37) (Ataxia telangiecta | Q13315 | 350417.1 | 4.51E−03 |
| 85 | ROCK1_HUMAN (Q13464) Rho-associated protein kinase 1 (EC 2.7.1.37) (Rho-assoc | Q13464 | 158074.9 | 1.42E−03 |
| 86 | ENPP2_HUMAN (Q13822) Ectonucleotide pyrophosphatase/phosphodiesterase 2 (E- | Q13822 | 98939.61 | 5.80E−10 |
| 87 | SMC1A_HUMAN (Q14683) Structural maintenance of chromosome 1-like 1 protein (S | Q14683 | 143143.6 | 9.76E−04 |
| 88 | NOLC1_HUMAN (Q14978) Nucleolar phosphoprotein p130 (Nucleolar 130 kDa protein | Q14978 | 73676.72 | 1.80E−03 |
| 89 | PLEC1_HUMAN (Q15149) Plectin 1 (PLTN) (PCN) (Hemidesmosomal protein 1) (HD1 | Q15149 | 531411.9 | 2.61E−04 |
| 90 | K1H1_HUMAN (Q15323) Keratin, type I cuticular Ha1 (Hair keratin, type I Ha1) | Q15323 | 47202.1 | 1.36E−03 |
| 91 | NCOA1_HUMAN (Q15788) Nuclear receptor coactivator 1 (EC 2.3.1.48) (NCoA-1) (St | Q15788 | 156641.7 | 4.14E−04 |
| 92 | Q15813 (Q15813) Beta-tubulin cofactor E (Tubulin-specific chaperone e) | Q15813 | 59309.08 | 5.90E−03 |
| 93 | SCN9A_HUMAN (Q15858) Sodium channel protein type IX alpha subunit (Voltage-gat | Q15858 | 226193.9 | 6.73E−03 |
| 94 | Q495C9 (Q495C9) Hypothetical protein LOC149134 | Q495C9 | 13623.18 | 6.59E−03 |
| 95 | Q4G0M1 (Q4G0M1) FLJ37034 protein (Fragment) | Q4G0M1 | 14453.34 | 6.80E−03 |
| 96 | Q567P4 (Q567P4) Hypothetical protein (Fragment) | Q567P4 | 34603.27 | 2.58E−03 |
| 97 | LRC10_HUMAN (Q5BKY1) Leucine-rich repeat-containing protein 10 | Q5BKY1 | 31621.81 | 9.03E−04 |
| 98 | Q5H9K7 (Q5H9K7) Novel protein (Fragment) | Q5H9K7 | 38685.59 | 6.32E−03 |
| 99 | Q5H9K8 (Q5H9K8) Armadillo repeat containing, X-linked 4 (Fragment) | Q5H9K8 | 36484.3 | 7.72E−03 |
| 100 | Q5JRL1 (Q5JRL1) Novel protein | Q5JRL1 | 30747.36 | 6.73E−03 |
| 101 | Q5T364 (Q5T364) OTTHUMP00000022702 | Q5T364 | 101941 | 4.05E−03 |
| 102 | Q5T3B9 (Q5T3B9) Novel protein | Q5T3B9 | 45993.33 | 4.46E−03 |
| 103 | Q5TBP2 (Q5TBP2) Retinoblastoma-associated factor 600 (RBAF600) (Fragment) | Q5TBP2 | 106303.1 | 4.66E−03 |
| 104 | Q5VWL1 (Q5VWL1) Membrane-associated guanylate kinase-related 3 (MAGI-3) | Q5VWL1 | 162847.8 | 7.51E−03 |
| 105 | Q5XLJ0 (Q5XLJ0) AIDA1C transcript variant 4 | Q5XLJ0 | 48400.84 | 9.99E−03 |
| 106 | Q6DN23 (Q6DN23) Platelet phospholipase A2 | Q6DN23 | 16135.78 | 4.11E−03 |
| 107 | Q6IAM5 (Q6IAM5) SLC27A6 protein | Q6IAM5 | 70042.55 | 3.12E−03 |
| 108 | Q6JIC5 (Q6JIC5) Cementum attachment protein | Q6JIC5 | 14910.42 | 9.87E−03 |
| 109 | Q6MZX7 (Q6MZX7) Hypothetical protein DKFZp686M24218 | Q6MZX7 | 52387.13 | 1.00E−30 |
| 110 | Q6P2C5 (Q6P2C5) PHF2 protein | Q6P2C5 | 35720.63 | 5.50E−03 |
| 111 | Q6P528 (Q6P528) ASPN protein | Q6P528 | 43863.48 | 8.71E−03 |
| 112 | POTE2_HUMAN (Q6S8J3) Prostate, ovary, testis expressed protein on chromosome 2 | Q6S8J3 | 80701.5 | 6.93E−03 |
| 113 | Q6UXZ8 (Q6UXZ8) FVSY9334 | Q6UXZ8 | 24230.68 | 7.07E−04 |
| 114 | Q6ZN08 (Q6ZN08) Hypothetical protein FLJ16537 | Q6ZN08 | 38813.18 | 4.37E−04 |
| 115 | Q6ZR14 (Q6ZR14) Hypothetical protein FLJ46736 | Q6ZR14 | 126662.9 | 8.79E−03 |
| 116 | Q6ZSK5 (Q6ZSK5) Hypothetical protein FLJ45433 | Q6ZSK5 | 15222.69 | 6.04E−03 |
| 117 | Q6ZSY5 (Q6ZSY5) Hypothetical protein FLJ45123 | Q6ZSY5 | 42154.05 | 7.90E−03 |
| 118 | Q6ZVQ3 (Q6ZVQ3) Hypothetical protein FLJ42220 | Q6ZVQ3 | 17595.21 | 1.14E−03 |
| 119 | Q6ZW49 (Q6ZW49) Hypothetical protein FLJ41606 | Q6ZW49 | 117615.6 | 3.89E−03 |
| 120 | Q70AK8 (Q70AK8) Ankyrin-repeat-ARM domain protein (Fragment) | Q70AK8 | 40496.33 | 8.67E−04 |
| 121 | Q70CQ2 (Q70CQ2) Ubiquitin-specific proteinase 34 | Q70CQ2 | 386873.7 | 9.41E−03 |
| 122 | Q76E79 (Q76E79) Mitochondrial methionyl-tRNA synthetase | Q76E79 | 66584.84 | 2.95E−03 |
| 123 | Q7L7Q2 (Q7L7Q2) BLOM7 beta (Novel protein) | Q7L7Q2 | 59101.39 | 1.49E−04 |
| 124 | Q7Z3P5 (Q7Z3P5) Hypothetical protein DKFZp686P13218 | Q7Z3P5 | 30940.46 | 5.90E−04 |
| 125 | NPHP3_HUMAN (Q7Z494) Nephrocystin-3 | Q7Z494 | 150768.9 | 5.74E−03 |
| 126 | Q86VG6 (Q86VG6) Hypothetical protein | Q86VG6 | 11523.95 | 3.43E−03 |
| 127 | TXND2_HUMAN (Q86VQ3) Thioredoxin domain-containing protein 2 (Spermatid-speci | Q86VQ3 | 60424.09 | 7.89E−04 |
| 128 | NAL14_HUMAN (Q86W24) NACHT-, LRR- and PYD-containing protein 14 (Nucleotide | Q86W24 | 124651.5 | 1.88E−03 |
| 129 | Q86WW8 (Q86WW8) Hypothetical protein MGC52110 (Hypothetical protein tmp_locu | Q86WW8 | 8370.071 | 4.59E−03 |
| 130 | Q86Y92 (Q86Y92) Similar to KIAA0922 protein (Fragment) | Q86Y92 | 154618.6 | 2.24E−03 |
| 131 | Q8IV73 (Q8IV73) Guanine nucleotide-releasing factor 2, isoform b | Q8IV73 | 122658.6 | 3.40E−03 |
| 132 | Q8IVL1 (Q8IVL1) Steerin2 protein | Q8IVL1 | 267950.3 | 2.98E−03 |
| 133 | Q8IVM4 (Q8IVM4) Hypothetical protein | Q8IVM4 | 6014.06 | 8.10E−03 |
| 134 | FA20C_HUMAN (Q8IXL6) Protein FAM20C precursor | Q8IXL6 | 64405.57 | 5.31E−03 |
| 135 | Q8IY66 (Q8IY66) DNA ligase IV (Ligase IV, DNA, ATP-dependent) | Q8IY66 | 103904 | 7.76E−03 |
| 136 | GP115_HUMAN (Q8IZF3) Probable G-protein coupled receptor 115 (G-protein couple | Q8IZF3 | 83775.66 | 5.14E−04 |
| 137 | Q8IZQ1 (Q8IZQ1) ALFY | Q8IZQ1 | 394992 | 6.15E−03 |
| 138 | AHI1_HUMAN (Q8N157) Jouberin (Abelson helper integration site 1 protein homolo | Q8N157 | 137029 | 2.76E−03 |
| 139 | Q8N1K5 (Q8N1K5) Hypothetical protein FLJ40584 | Q8N1K5 | 73625.31 | 5.82E−03 |
| 140 | Q8N3I3 (Q8N3I3) Hypothetical protein DKFZp761P18121 | Q8N3I3 | 81550.7 | 3.18E−03 |
| 141 | Q8N543 (Q8N543) Hypothetical protein FLJ10826 | Q8N543 | 63206.21 | 1.90E−03 |
| 142 | Q8N998 (Q8N998) Hypothetical protein FLJ38159 | Q8N998 | 43781.59 | 7.01E−03 |
| 143 | Q8N9S2 (Q8N9S2) Hypothetical protein FLJ36635 | Q8N9S2 | 28973.04 | 5.25E−03 |
| 144 | Q8NAJ6 (Q8NAJ6) Hypothetical protein FLJ35251 | Q8NAJ6 | 81745.5 | 1.24E−03 |
| 145 | Q8NCU4 (Q8NCU4) Hypothetical protein | Q8NCU4 | 110499 | 9.83E−03 |
| 146 | Q8NDA2 (Q8NDA2) Hypothetical protein DKFZp434P0216 (Fragment) | Q8NDA2 | 143799.3 | 7.52E−04 |
| 147 | Q8TAN9 (Q8TAN9) Oxoglutarate dehydrogenase-like | Q8TAN9 | 114423.7 | 6.91E−03 |
| 148 | Q8TBB5 (Q8TBB5) Kelch domain containing 4 | Q8TBB5 | 57855.43 | 1.01E−03 |
| 149 | Q8TBZ0 (Q8TBZ0) Hypothetical protein KM-HN-1 | Q8TBZ0 | 96664.98 | 6.67E−04 |
| 150 | Q8TC05 (Q8TC05) Mdm4, transformed 3T3 cell double minute 1, p53 binding protein | Q8TC05 | 80695.09 | 5.19E−03 |
| 151 | CHD6_HUMAN (Q8TD26) Chromodomain-helicase-DNA-binding protein 6 (CHD-6) (R | Q8TD26 | 305218.9 | 6.52E−03 |
| 152 | PAR3L_HUMAN (Q8TEW8) Amyotrophic lateral sclerosis 2 chromosome region candi | Q8TEW8 | 132412.5 | 9.52E−03 |
| 153 | Q8WVY7 (Q8WVY7) Ubiquitin-like domain containing CTD phosphatase 1 (CTD-like p | Q8WVY7 | 36781.22 | 8.32E−04 |
| 154 | ANKR7_HUMAN (Q92527) Ankyrin repeat domain protein 7 (Testis-specific protein | Q92527 | 22573.66 | 8.25E−03 |
| 155 | Q92735 (Q92735) FMI protein | Q92735 | 161492.6 | 4.05E−03 |
| 156 | SNPC3_HUMAN (Q92966) snRNA-activating protein complex subunit 3 (SNAPc subur | Q92966 | 46722.65 | 9.07E−03 |
| 157 | IRF7_HUMAN (Q92985) Interferon regulatory factor 7 (IRF-7) | Q92985 | 54244.25 | 7.33E−04 |
| 158 | Q96AG4 (Q96AG4) Hypothetical protein PRO1855 | Q96AG4 | 34908.9 | 9.06E−03 |
| 159 | ISG20_HUMAN (Q96AZ6) Interferon-stimulated gene 20 kDa protein (EC 3.1.13.1) ( | Q96AZ6 | 20350.62 | 9.06E−05 |
| 160 | GMCL1_HUMAN (Q96IK5) Germ cell-less protein-like 1 | Q96IK5 | 58647.47 | 7.74E−03 |
| 161 | KAD7_HUMAN (Q96M32) Putative adenylate kinase 7 (EC 2.7.4.3) | Q96M32 | 82620.65 | 9.64E−03 |
| 162 | DB118_HUMAN (Q96PH6) Beta-defensin 118 precursor (Beta-defensin 18) (DEFB-18 | Q96PH6 | 13604.79 | 5.68E−03 |
| 163 | VPS35_HUMAN (Q96QK1) Vacuolar protein sorting 35 (Vesicle protein sorting 35) | Q96QK1 | 91649.07 | 4.26E−03 |
| 164 | MAGI1_HUMAN (Q96QZ7) Membrane associated guanylate kinase, WW and PDZ do | Q96QZ7 | 164540.2 | 7.80E−03 |
| 165 | MCCA_HUMAN (Q96RQ3) Methylcrotonoyl-CoA carboxylase alpha chain, mitochondri | Q96RQ3 | 80382.04 | 3.52E−03 |
| 166 | NIBL_HUMAN (Q96TA1) Niban-like protein (Meg-3) | Q96TA1 | 82631.11 | 6.21E−03 |
| 167 | SPS2_HUMAN (Q99611) Selenide, water dikinase 2 (EC 2.7.9.3) (Selenophosphate s) | Q99611 | 47227.85 | 2.61E−03 |
| 168 | KIF2C_HUMAN (Q99661) Kinesin-like protein KIF2C (Mitotic centromere-associated | Q99661 | 81261.21 | 5.02E−03 |
| 169 | CLP24_HUMAN (Q9BSN7) Claudin-like protein 24 | Q9BSN7 | 24523.55 | 2.48E−03 |
| 170 | DATF1_HUMAN (Q9BTC0) Death-associated transcription factor 1 | Q9BTC0 | 129070.5 | 7.61E−03 |
| 171 | CHST6_HUMAN (Q9GZX3) Carbohydrate sulfotransferase 6 (EC 2.8.2.—) (N-acetylglu | Q9GZX3 | 44071.2 | 5.32E−03 |
| 172 | TNKS2_HUMAN (Q9H2K2) Tankyrase 2 (EC 2.4.2.30) (TANK2) (Tankyrase II) (TNKS | Q9H2K2 | 126838.5 | 1.90E−05 |
| 173 | Q9H2M8 (Q9H2M8) DC28 | Q9H2M8 | 64005.01 | 8.59E−03 |
| 174 | SMRCD_HUMAN (Q9H4L7) SWI/SNF-related, matrix associated, actin-dependent reg | Q9H4L7 | 117299.9 | 7.44E−03 |
| 175 | Q9H9B0 (Q9H9B0) Hypothetical protein FLJ12883 | Q9H9B0 | 91169.25 | 9.96E−03 |
| 176 | Q9H9B6 (Q9H9B6) Hypothetical protein FLJ12873 | Q9H9B6 | 71307.3 | 4.63E−03 |
| 177 | LPHN3_HUMAN (Q9HAR2) Latrophilin-3 precursor (Calcium-independent alpha-latrot | Q9HAR2 | 161709.3 | 9.90E−03 |
| 178 | MAGE1_HUMAN (Q9HCI5) Melanoma-associated antigen E1 (MAGE-E1 antigen) (He | Q9HCI5 | 103189.7 | 6.56E−03 |
| 179 | OLFL3_HUMAN (Q9NRN5) Olfactomedin-like protein 3 precursor (HNOEL-iso) (hOLF | Q9NRN5 | 45981.32 | 1.86E−03 |
| 180 | TB22B_HUMAN (Q9NU19) TBC1 domain family member 22B | Q9NU19 | 59044.32 | 3.31E−03 |
| 181 | CT038_HUMAN (Q9NUV7) Protein C20orf38 | Q9NUV7 | 19679.8 | 7.71E−03 |
| 182 | Q9NXE3 (Q9NXE3) Hypothetical protein FLJ20298 | Q9NXE3 | 26243.39 | 8.80E−04 |
| 183 | THUM1_HUMAN (Q9NXG2) THUMP domain containing protein 1 | Q9NXG2 | 39290.82 | 7.48E−03 |
| 184 | TE2IP_HUMAN (Q9NYB0) Telomeric repeat binding factor 2 interacting protein 1 ( | Q9NYB0 | 44232.88 | 1.44E−03 |
| 185 | ZN226_HUMAN (Q9NYT6) Zinc finger protein 226 | Q9NYT6 | 91861.71 | 4.95E−03 |
| 186 | GSCR1_HUMAN (Q9NZM4) Glioma tumor suppressor candidate region gene 1 protein | Q9NZM4 | 152898.4 | 6.54E−03 |
| 187 | HCN3_HUMAN (Q9P1Z3) Potassium/sodium hyperpolarization-activated cyclic nucleo | Q9P1Z3 | 85977.23 | 5.36E−03 |
| 188 | CEP72_HUMAN (Q9P209) Centrosomal protein of 72 kDa (Cep72 protein) | Q9P209 | 71673.01 | 1.96E−03 |
| 189 | CING_HUMAN (Q9P2M7) Cingulin | Q9P2M7 | 136303.7 | 5.06E−03 |
| 190 | SL9A2_HUMAN (Q9UBY0) Sodium/hydrogen exchanger 2 (Na(+)/H(+) exchanger 2) (I | Q9UBY0 | 91461.34 | 6.23E−04 |
| 191 | Q9UJU1 (Q9UJU1) Cytovillin 2 (Fragment) | Q9UJU1 | 16251.62 | 8.20E−03 |
| 192 | STML2_HUMAN (Q9UJZ1) Stomatin-like protein 2 (SLP-2) (EPB72-like 2) | Q9UJZ1 | 38510.22 | 2.48E−03 |
| 193 | MYH13_HUMAN (Q9UKX3) Myosin-13 (Myosin heavy chain, skeletal muscle, extraoc | Q9UKX3 | 223539.2 | 5.60E−03 |
| 194 | TTC7A_HUMAN (Q9ULT0) Tetratricopeptide repeat protein 7A (TPR repeat protein 7 | Q9ULT0 | 96123.27 | 6.23E−03 |
| 195 | ALK_HUMAN (Q9UM73) ALK tyrosine kinase receptor precursor (EC 2.7.1.112) (Anap | Q9UM73 | 176304.4 | 9.57E−03 |
| 196 | ICAM5_HUMAN (Q9UMF0) Intercellular adhesion molecule 5 precursor (ICAM-5) (Tel | Q9UMF0 | 97270.1 | 2.21E−03 |
| 197 | SOX13_HUMAN (Q9UN79) SOX-13 protein (Type 1 diabetes autoantigen ICA12) (Isle | Q9UN79 | 98718.72 | 7.41E−03 |
| 198 | CNTN6_HUMAN (Q9UQ52) Contactin 6 precursor (Neural recognition molecule NB-3) | Q9UQ52 | 113885.1 | 5.55E−03 |
| 199 | NALDL_HUMAN (Q9UQQ1) N-acetylated-alpha-linked acidic dipeptidase-like protein | Q9UQQ1 | 80574.27 | 1.21E−03 |
| 200 | CG141_HUMAN (Q9Y3E0) UPF0198 protein CGI-141 | Q9Y3E0 | 15415.4 | 5.94E−05 |
| 201 | ZN337_HUMAN (Q9Y3M9) Zinc finger protein 337 | Q9Y3M9 | 86819.26 | 4.48E−03 |
| 202 | TLN1_HUMAN (Q9Y490) Talin-1 | Q9Y490 | 269596.3 | 3.76E−03 |
| 203 | PCDGB_HUMAN (Q9Y5H2) Protocadherin gamma A11 precursor (PCDH-gamma-A1 | Q9Y5H2 | 101480.6 | 9.51E−03 |
| 204 | WIF1_HUMAN (Q9Y5W5) Wnt inhibitory factor 1 precursor (WIF-1) | Q9Y5W5 | 41499.8 | 5.15E−03 |
| 205 | S12A7_HUMAN (Q9Y666) Solute carrier family 12 member 7 (Electroneutral potassiu | Q9Y666 | 119072.7 | 1.59E−04 |
| 206 | SNCAP_HUMAN (Q9Y6H5) Synphilin-1 (Alpha-synuclein-interacting protein) | Q9Y6H5 | 100319 | 5.97E−03 |
| 413 | DC13_HUMAN (Q9NRP2) UPF0287 protein DC13 | Q9NRP2 | 9453.72 | 2.81E−03 |
| 414 | Q9NS87 (Q9NS87) Kinesin-like protein 2 | Q9NS87 | 160060.4 | 4.45E−03 |
| 415 | Q9NSN6 (Q9NSN6) Hypothetical protein DKFZp761O0113 | Q9NSN6 | 39970.1 | 6.56E−03 |
| 416 | EMIL3_HUMAN (Q9NT22) EMILIN-3 precursor (EMILIN-5) (Elastin microfibril interf | Q9NT22 | 82595.73 | 3.69E−03 |
| 417 | UGGG1_HUMAN (Q9NYU2) UDP-glucose:glycoprotein glucosyltransferase 1 precurs | Q9NYU2 | 174866.5 | 3.83E−03 |
| 418 | T2R14_HUMAN (Q9NYV8) Taste receptor type 2 member 14 (T2R14) (Taste receptor | Q9NYV8 | 36136.48 | 1.33E−03 |
| 419 | COMD9_HUMAN (Q9P000) COMM domain containing protein 9 | Q9P000 | 21904.53 | 4.29E−04 |
| 420 | VPS18_HUMAN (Q9P253) Vacuolar protein sorting 18 (hVPS18) | Q9P253 | 110115.9 | 7.01E−03 |
| 421 | RRBP1_HUMAN (Q9P2E9) Ribosome-binding protein 1 (Ribosome receptor protein) ( | Q9P2E9 | 152380 | 4.24E−03 |
| 422 | SUCB1_HUMAN (Q9P2R7) Succinyl-CoA ligase [ADP-forming] beta-chain, mitochond | Q9P2R7 | 50299.33 | 8.47E−03 |
| 423 | GT2D1_HUMAN (Q9UHL9) General transcription factor II-1 repeat domain-containin | Q9UHL9 | 105990.8 | 4.56E−03 |
| 424 | Q9UI74 (Q9UI74) PRO0245 | Q9UI74 | 8075.004 | 7.12E−04 |
| 425 | SPB13_HUMAN (Q9UIV8) Serpin B13 (Hurpin) (HaCaT UV-repressible serpin) (Prote | Q9UIV8 | 44248.2 | 8.72E−04 |
| 426 | HOOK1_HUMAN (Q9UJC3) Hook homolog 1 (h-hook1) (hHK1) | Q9UJC3 | 84594.84 | 9.77E−03 |
| 427 | DKKL1_HUMAN (Q9UK85) Dickkopf-like protein 1 precursor (Soggy-1 protein) (SGY-1 | Q9UK85 | 26990.23 | 1.36E−04 |
| 428 | PADI3_HUMAN (Q9ULW8) Protein-arginine deiminase type III (EC 3.5.3.15) (Peptid | Q9ULW8 | 74695.37 | 9.91E−04 |
| 429 | MLL4_HUMAN (Q9UMN6) Myeloid/lymphoid or mixed-lineage leukemia protein 4 (Trit | Q9UMN6 | 293327.3 | 9.43E−03 |
| 430 | GPR34_HUMAN (Q9UPC5) Probable G-protein coupled receptor 34 | Q9UPC5 | 43831.09 | 8.71E−03 |
| 431 | Q9UQ10 (Q9UQ10) Dimeric dihydrodiol dehydrogenase (EC 1.3.1.20) | Q9UQ10 | 36358.77 | 9.73E−03 |
| 432 | SHOC2_HUMAN (Q9UQ13) Leucine-rich repeat protein SHOC-2 (Ras-binding protein | Q9UQ13 | 64847.64 | 3.26E−03 |
| 433 | AT11B_HUMAN (Q9Y2G3) Probable phospholipid-transporting ATPase IF (EC 3.6.3.1 | Q9Y2G3 | 134104.1 | 9.84E−03 |
| 434 | PCDG7_HUMAN (Q9Y5G6) Protocadherin gamma A7 precursor (PCDH-gamma-A7) | Q9Y5G6 | 101659.2 | 2.96E−03 |
| 435 | PCDA2_HUMAN (Q9Y5H9) Protocadherin alpha 2 precursor (PCDH-alpha2) | Q9Y5H9 | 102000.4 | 6.26E−03 |
| 436 | Q9Y5S5 (Q9Y5S5) DNA polymerase epsilon catalytic subunit protein isoform a | Q9Y5S5 | 262846.1 | 9.10E−03 |
| 437 | SO1B1_HUMAN (Q9Y6L6) Solute carrier organic anion transporter family member 1B | Q9Y6L6 | 76398.98 | 1.51E−03 |

TABLE 10
Vitreous proteins identified from patients with Macular Hole (MH)

| | Protein | Accession | MW | P (pro) |
|---|---|---|---|---|
| 207 | Protein KIAA1683 | Q9H0B3 | 127611.8 | 2.65E−04 |
| 208 | Apolipoprotein A-IV precursor | P06727 | 45343.52 | 4.69E−12 |
| 209 | KIAA0425 protein | O43308 | 141056.5 | 2.12E−04 |
| 210 | MYH7B protein | Q96I57 | 114574.5 | 5.17E−04 |
| 211 | Sortilin-related receptor precursor | Q92673 | 248280 | 2.94E−04 |
| 212 | Ig kappa chain V-II region | P01614 | 12668.32 | 4.45E−07 |
| 213 | Ig kappa chain V-II region MIL | P01616 | 12048.05 | 4.45E−07 |
| 214 | Piccolo protein (Aczonin) | Q9Y6V0 | 566313.1 | 4.86E−04 |
| 215 | Alpha-1B-glycoprotein precursor | P04217 | 54238.7 | 1.32E−09 |
| 216 | Neuronal membrane glycoprotein M6-a | P51674 | 31188.39 | 4.39E−03 |
| 217 | Nuclear pore complex protein Nup153 | P49790 | 153793.4 | 5.51E−03 |
| 218 | Secreted frizzled-related protein 3 precursor | Q92765 | 36230.29 | 5.30E−06 |
| 219 | A-kinase anchor protein 3 | O75969 | 94676.41 | 2.47E−05 |
| 220 | Telomeric repeat binding factor 1 | P54274 | 50313.11 | 2.43E−03 |
| 221 | Kinesin-like protein KIF1A | Q12756 | 190963.1 | 3.48E−03 |
| 222 | UDP-N-acetylhexosamine pyrophosphorylase | Q16222 | 58731.98 | 2.88E−03 |
| 223 | RIF1_HUMAN (Q5UIP0) Telomere-associated protein RIF1 (Rap1-interacting factor | Q5UIP0 | 274293 | 2.56E−03 |
| 224 | Q6P4H8 (Q6P4H8) Hypothetical protein LOC134145 | Q6P4H8 | 26123.47 | 3.97E−03 |
| 225 | Q8IV47 (Q8IV47) Fibromodulin, | Q8IV47 | 43151.57 | 6.22E−04 |
| 226 | Q8N9V7 (Q8N9V7) Hypothetical protein FLJ36157 | Q8N9V7 | 77526.28 | 6.30E−04 |
| 227 | LIPB2_HUMAN (Q8ND30) Liprin-beta 2 (Protein tyrosine phosphatase receptor type | Q8ND30 | 98383.37 | 3.34E−03 |
| 228 | SYNE1_HUMAN (Q8NF91) Nesprin-1 (Nuclear envelope spectrin repeat protein 1) (Sy | Q8NF91 | 1010433 | 1.60E−03 |
| 229 | K1849_HUMAN (Q96JH8) Protein KIAA1849 | Q96JH8 | 116630 | 6.35E−03 |
| 230 | GGA2_HUMAN (Q9UJY4) ADP-ribosylation factor binding protein GGA2 (Golgi-locali | Q9UJY4 | 67133.75 | 3.44E−03 |
| 231 | Q9ULH9 (Q9ULH9) KIAA1241 protein (Fragment) | Q9ULH9 | 97158.45 | 1.73E−05 |
| 232 | CT004_HUMAN (Q9Y312) Protein C20orf4 | Q9Y312 | 43444.13 | 5.11E−03 |
| 233 | KIF3A_HUMAN (Q9Y496) Kinesin-like protein KIF3A (Microtubule plus end-directed | Q9Y496 | 80335.11 | 8.14E−04 |
| 234 | CAC1G_HUMAN (O43497) Voltage-dependent T-type calcium channel alpha-1G subu | O43497 | 262301.1 | 2.51E−03 |
| 235 | GLU2B_HUMAN (P14314) Glucosidase II beta subunit precursor (Protein kinase C s | P14314 | 59258.88 | 1.97E−03 |
| 236 | HBG1_HUMAN (P69891) Hemoglobin gamma-1 subunit (Hemoglobin gamma-1 chain | P69891 | 15999.27 | 8.59E−03 |
| 237 | APXL_HUMAN (Q13796) Apical-like protein (APXL protein) | Q13796 | 176302 | 1.55E−03 |
| 238 | UBP10_HUMAN (Q14694) Ubiquitin carboxyl-terminal hydrolase 10 (EC 3.1.2.15) (U | Q14694 | 87079.84 | 1.05E−03 |
| 239 | Q15394 (Q15394) KIAA0005 protein (Fragment) | Q15394 | 48615.16 | 6.56E−04 |
| 240 | Q4V9L7 (Q4V9L7) NGRN protein (Fragment) | Q4V9L7 | 31332.13 | 4.36E−03 |
| 241 | Q53SW1 (Q53SW1) Hypothetical protein FLJ20254 | Q53SW1 | 48539.34 | 2.53E−03 |
| 242 | Q5BLQ2 (Q5BLQ2) Mucin | Q5BLQ2 | 289658.2 | 5.22E−03 |
| 243 | Q5JRC3 (Q5JRC3) RPEL repeat containing 1 (Fragment) | Q5JRC3 | 25543.2 | 4.33E−03 |
| 244 | Q5T2J8 (Q5T2J8) OTTHUMP00000018139 (Fragment) | Q5T2J8 | 97147.08 | 1.44E−03 |
| 245 | Q65ZC8 (Q65ZC8) Single-chain Fv (Fragment) | Q65ZC8 | 26110.55 | 3.05E−05 |
| 246 | VPS36_HUMAN (Q86VN1) Vacuolar protein sorting protein 36 (ELL-associated protei | Q86VN1 | 43788.79 | 1.09E−03 |
| 247 | RYR2_HUMAN (Q92736) Ryanodine receptor 2 (Cardiac muscle-type ryanodine recep | Q92736 | 564136.1 | 6.98E−03 |
| 248 | TOP1M_HUMAN (Q969P6) DNA topoisomerase I, mitochondrial precursor (EC 5.99.1. | Q969P6 | 69828.34 | 5.03E−03 |
| 249 | Q96EK0 (Q96EK0) Hypothetical protein MGC4655 | Q96EK0 | 41865.86 | 1.44E−03 |
| 250 | Q9BRQ8 (Q9BRQ8) Apoptosis-inducing factor (AIF)-like mitchondrion-associated i | Q9BRQ8 | 40501.28 | 2.48E−03 |
| 251 | Q9BS34 (Q9BS34) Zinc finger protein 670 (Hypothetical protein FLJ90293) (Novel | Q9BS34 | 44574.27 | 7.16E−03 |
| 252 | PLEA5_HUMAN (Q9HAU0) Pleckstrin homology domain-containing protein family A m | Q9HAU0 | 127384.5 | 3.50E−03 |
| 253 | Q5VXU2 (QSVXU2) Propionyl Coenzyme A carboxylase, alpha polypeptide | Q5VXU2 | 80235.37 | 1.34E−04 |
| 254 | SGK1_HUMAN (O00141) Serine/threonine-protein kinase Sgk1 (EC 2.7.1.37) (Serum/ | O00141 | 48925.35 | 1.08E−03 |
| 255 | O14913 (O14913) Kruppel-associated box protein | O14913 | 52045.66 | 2.39E−03 |
| 256 | HAT1_HUMAN (O14929) Histone acetyltransferase type B catalytic subunit (EC 2.3. | O14929 | 49480.84 | 1.43E−03 |
| 257 | O15014 (O15014) KIAA0295 protein (Fragment) | O15014 | 106382.5 | 2.79E−03 |
| 258 | PDZK3_HUMAN (O15018) PDZ domain containing protein 3 (PDZ domain containing I | O15018 | 301423.4 | 2.91E−03 |
| 259 | DMN_HUMAN (O15061) Desmuslin | O15061 | 172662.5 | 2.87E−03 |
| 260 | SOX12_HUMAN (O15370) SOX-12 protein (SOX-22 protein) | O15370 | 34279.67 | 1.92E−03 |
| 261 | O43719 (O43719) HIV TAT specific factor 1 | O43719 | 85800.38 | 2.48E−03 |
| 262 | KPRB_HUMAN (O60256) Phosphoribosyl pyrophosphate synthetase-associated protei | O60256 | 40899.39 | 3.63E−05 |
| 263 | LSD1_HUMAN (O60341) Lysine-specific histone demethylase 1 (EC 1.—.—.—) (Amine | O60341 | 92844.88 | 2.99E−04 |
| 264 | FZD6_HUMAN (O60353) Frizzled 6 precursor (Frizzled-6) (Fz-6) (hFz6) | O60353 | 79222.06 | 1.13E−03 |
| 265 | O60826 (O60826) JM1 protein (Hypothetical protein CCDC22) | O60826 | 70712.32 | 2.74E−03 |
| 266 | GAS7_HUMAN (O60861) Growth-arrest-specific protein 7 (GAS-7) | O60861 | 47236.75 | 3.84E−04 |
| 267 | O75179 (O75179) KIAA0697 protein (Fragment) | O75179 | 263079.2 | 1.34E−03 |
| 268 | PRPU_HUMAN (O94906) U5 snRNP-associated 102 kDa protein (U5-102 kDa protein | O94906 | 106857.9 | 8.74E−03 |
| 269 | SLIK3_HUMAN (O94933) SLIT and NTRK-like protein 3 precursor | O94933 | 108937.1 | 9.29E−03 |
| 270 | O95285 (O95285) Erythroblast macrophage protein EMP | O95285 | 43879.31 | 8.16E−03 |
| 271 | FMNL_HUMAN (O95466) Formin-like 1 protein (Formin-like protein) (Leukocyte for | O95466 | 121750.9 | 9.19E−03 |
| 272 | HPT_HUMAN (P00738) Haptoglobin precursor [Contains: Haptoglobin alpha chain; H | P00738 | 45176.59 | 2.71E−05 |
| 273 | KV2B_HUMAN (P01615) Ig kappa chain V-II region FR | P01615 | 12651.75 | 5.81E−04 |
| 274 | CO3A1_HUMAN (P02461) Collagen alpha 1(III) chain precursor | P02461 | 138470.2 | 4.25E−03 |
| 275 | FIBG_HUMAN (P02679) Fibrinogen gamma chain precursor | P02679 | 51478.88 | 1.61E−04 |
| 276 | CRP_HUMAN (P02741) C-reactive protein precursor [Contains: C-reactive protein( | P02741 | 25022.68 | 9.82E−03 |
| 277 | AMBP_HUMAN (P02760) AMBP protein precursor [Contains: Alpha-1-microglobulin (P | P02760 | 38973.99 | 1.37E−04 |
| 278 | CSF1R_HUMAN (P07333) Macrophage colony-stimulating factor 1 receptor precursor | P07333 | 107915.2 | 8.66E−03 |
| 279 | RET_HUMAN (P07949) Proto-oncogene tyrosine-protein kinase receptor ret precurs | P07949 | 124239.4 | 4.41E−03 |
| 280 | RARA_HUMAN (P10276) Retinoic acid receptor alpha (RAR-alpha) | P10276 | 50738.42 | 5.40E−04 |
| 281 | C1TC_HUMAN (P11586) C-1-tetrahydrofolate synthase, cytoplasmic (C1-THF synthas | P11586 | 101364.5 | 2.60E−03 |
| 282 | CSPG2_HUMAN (P13611) Versican core protein precursor (Large fibroblast proteog | P13611 | 372589 | 7.15E−06 |
| 283 | K1C13_HUMAN (P13646) Keratin, type I cytoskeletal 13 (Cytokeratin-13) (CK-13) | P13646 | 49555.45 | 4.22E−03 |
| 284 | NEBU_HUMAN (P20929) Nebulin | P20929 | 772742.8 | 7.84E−04 |
| 285 | PRDX2_HUMAN (P32119) Peroxiredoxin 2 (EC 1.11.1.15) (Thioredoxin peroxidase 1) | P32119 | 21747.2 | 9.46E−03 |
| 286 | MYH11_HUMAN (P35749) Myosin-11 (Myosin heavy chain, smooth muscle isoform) ( | P35749 | 227197.9 | 2.94E−03 |
| 287 | BRCA1_HUMAN (P38398) Breast cancer type 1 susceptibility protein (RING finger | P38398 | 207590.9 | 9.37E−03 |
| 288 | RL13A_HUMAN (P40429) 60S ribosomal protein L13a (23 kDa highly basic protein) | P40429 | 23431.35 | 9.48E−03 |
| 289 | STAT1_HUMAN (P42224) Signal transducer and activator of transcription 1-alpha/ | P42224 | 87279.63 | 7.68E−03 |
| 290 | RHG25_HUMAN (P42331) Rho-GTPase-activating protein 25 | P42331 | 72385.19 | 8.14E−03 |
| 291 | AFAM_HUMAN (P43652) Afamin precursor (Alpha-albumin) (Alpha-Alb) | P43652 | 69024.09 | 8.12E−05 |
| 292 | RBP2_HUMAN (P49792) Ran-binding protein 2 (RanBP2) (Nuclear pore complex prot | P49792 | 357991.4 | 3.16E−03 |
| 293 | DPOG1_HUMAN (P54098) DNA polymerase gamma subunit 1 (EC 2.7.7.7) (Mitochon | P54098 | 139473.4 | 3.35E−03 |
| 294 | ELL_HUMAN (P55199) RNA polymerase II elongation factor ELL (Eleven-nineteen ly | P55199 | 68222.91 | 5.19E−03 |
| 295 | CAD12_HUMAN (P55289) Brain-cadherin precursor (BR-cadherin) (Cadherin-12) (N-c | P55289 | 88219.76 | 1.52E−03 |
| 296 | KR102_HUMAN (P60368) Keratin-associated protein 10-2 (Keratin-associated prote | P60368 | 25595.75 | 1.72E−03 |
| 297 | RT21_HUMAN (P82921) Mitochondrial 28S ribosomal protein S21 (S21mt) (MRP-S21 | P82921 | 10734.48 | 2.34E−03 |
| 298 | NSBP1_HUMAN (P82970) Nucleosomal binding protein 1 | P82970 | 31505.95 | 1.93E−03 |
| 299 | SCN7A_HUMAN (Q01118) Sodium channel protein type VII alpha subunit (Putative v | Q01118 | 193345 | 1.66E−03 |
| 300 | BDH_HUMAN (Q02338) D-beta-hydroxybutyrate dehydrogenase, mitochondrial precur | Q02338 | 38132.47 | 5.68E−03 |
| 301 | CK013_HUMAN (Q02833) Protein C11orf13 (HRAS1-related cluster protein 1) | Q02833 | 39920.6 | 3.17E−03 |
| 302 | BPA1_HUMAN (Q03001) Bullous pemphigoid antigen 1 isoforms 1/2/3/4/5/8 (230 kDa | Q03001 | 371976.9 | 7.39E−03 |
| 303 | ATP7A_HUMAN (Q04656) Copper-transporting ATPase 1 (EC 3.6.3.4) (Copper pump | Q04656 | 163230 | 3.35E−03 |
| 304 | APLP2_HUMAN (Q06481) Amyloid-like protein 2 precursor (Amyloid protein homolog | Q06481 | 86900.28 | 1.03E−09 |
| 305 | TRDN_HUMAN (Q13061) Triadin | Q13061 | 81374.51 | 6.20E−03 |
| 306 | CSN1_HUMAN (Q13098) COP9 signalosome complex subunit 1 (Signalosome subuni | Q13098 | 53338.1 | 7.31E−03 |
| 307 | NOG2_HUMAN (Q13823) Nucleolar GTP-binding protein 2 (Autoantigen NGP-1) | Q13823 | 83603.43 | 9.59E−03 |
| 308 | Q14159 (Q14159) KIAA0146 protein (Fragment) | Q14159 | 100592.3 | 1.44E−03 |
| 309 | BMS1_HUMAN (Q14692) Ribosome biogenesis protein BMS1 homolog | Q14692 | 145715.5 | 3.76E−03 |
| 310 | 2A5D_HUMAN (Q14738) Serine/threonine protein phosphatase 2A, 56 kDa regulatory | Q14738 | 69947.5 | 2.98E−03 |
| 311 | RBBP5_HUMAN (Q15291) Retinoblastoma-binding protein 5 (RBBP-5) (Retinoblastorr | Q15291 | 59044.69 | 9.99E−03 |
| 312 | CDC37_HUMAN (Q16543) Hsp90 co-chaperone Cdc37 (Hsp90 chaperone protein kin | Q16543 | 44440.05 | 8.92E−03 |
| 313 | Q49AA0 (Q49AA0) ZNF642 protein | Q49AA0 | 61112.29 | 5.50E−03 |
| 314 | Q4VC44 (Q4VC44) FLYWCH-type zinc finger 1, isoform a | Q4VC44 | 79985.74 | 1.11E−03 |
| 315 | Q4VXM4 (Q4VXM4) Chromosome 20 open reading frame 17 | Q4VXM4 | 114932.5 | 2.21E−03 |
| 316 | Q53EU6 (Q53EU6) Hypothetical protein (Fragment) | Q53EU6 | 48673.52 | 9.80E−04 |
| 317 | Q53RG2 (Q53RG2) Hypothetical protein CHRNG | Q53RG2 | 58259.47 | 4.34E−03 |
| 318 | Q562E5 (Q562E5) KIAA0194 protein (Fragment) | Q562E5 | 157553.1 | 3.73E−04 |
| 319 | Q59FI9 (Q59FI9) Ribosomal protein L12 variant (Fragment) | Q59FI9 | 21469.49 | 6.72E−03 |
| 320 | Q5JPU1 (Q5JPU1) Pyruvate dehydrogenase (Lipoamide) alpha 1 (Fragment) | Q5JPU1 | 22743.74 | 3.07E−03 |
| 321 | Q5JTH6 (Q5JTH6) Exosomal core protein CSL4 | Q5JTH6 | 18804.54 | 5.99E−04 |
| 322 | Q5JX69 (Q5JX69) OTTHUMP00000031352 | Q5JX69 | 19485.96 | 8.08E−03 |
| 323 | Q5T1J6 (Q5T1J6) OTTHUMP00000030508 | Q5T1J6 | 17161.46 | 4.35E−03 |
| 324 | Q5TDG2 (Q5TDG2) Hydroxy-delta-5-steroid dehydrogenase, 3 beta-and steroid delt | Q5TDG2 | 42427.11 | 2.32E−03 |
| 325 | Q5TYW1 (Q5TYW1) Novel protein (Fragment) | Q5TYW1 | 97254.69 | 4.42E−03 |
| 326 | Q5VXI2 (Q5VXI2) Myosin IIIA | Q5VXI2 | 22191.69 | 7.99E−03 |
| 327 | Q5VYM8 (Q5VYM8) Unc-13 homolog B (C. elegans) | Q5VYM8 | 180563.2 | 2.31E−03 |
| 328 | Q5XPV5 (Q5XPV5) Sjogren syndrome antigen A1 | Q5XPV5 | 54089.2 | 5.68E−04 |
| 329 | Q63HP7 (Q63HP7) Hypothetical protein DKFZp686P1551 | Q63HP7 | 65621.97 | 5.39E−03 |
| 330 | Q659F3 (Q659F3) Hypothetical protein DKFZp434J194 | Q659F3 | 14956.27 | 4.86E−03 |
| 331 | CEP4_HUMAN (Q66GS9) Centrosomal protein 4 (Centrosomal protein of 135 kDa) (C | Q66GS9 | 133421.9 | 9.02E−04 |
| 332 | Q68CI8 (Q68CI8) Hypothetical protein HMFT1272 (Fragment) | Q68CI8 | 48598.88 | 2.38E−03 |
| 333 | Q68DL8 (Q68DL8) Hypothetical protein DKFZp781L0319 | Q68DL8 | 84645.59 | 3.98E−03 |
| 334 | S6A19_HUMAN (Q695T7) Sodium-dependent neutral amino acid transporter B(0) (Sys | Q695T7 | 71063.29 | 5.44E−03 |
| 335 | Q6A334 (Q6A334) Mutated in bladder cancer 1 | Q6A334 | 61882.69 | 8.74E−03 |
| 336 | Q6IA31 (Q6IA31) FLJ14154 protein | Q6IA31 | 27415.97 | 7.48E−03 |
| 337 | Q6IBW4 (Q6IBW4) Em: U62317.2 protein | Q6IBW4 | 68183.7 | 6.70E−03 |
| 338 | Q6IE81 (Q6IE81) JADE1L protein | Q6IE81 | 95472.82 | 9.07E−03 |
| 339 | Q6IF05 (Q6IF05) Olfactory receptor OR1-47 | Q6IF05 | 37737.82 | 8.84E−04 |
| 340 | ST6B1_HUMAN (Q6IMI4) Sulfotransferase 6B1 (EC 2.8.2.—) | Q6IMI4 | 30491.21 | 9.60E−03 |
| 341 | PTRF_HUMAN (Q6NZI2) Polymerase I and transcript release factor (PTRF protein) | Q6NZI2 | 43449.88 | 1.14E−03 |
| 342 | Q6P3W7 (Q6P3W7) Similar to mouse D10Ertd802e protein | Q6P3W7 | 103642.4 | 9.06E−04 |
| 343 | Q6P658 (Q6P658) FNBP1 protein (Fragment) | Q6P658 | 40759.38 | 6.14E−04 |
| 344 | GP133_HUMAN (Q6QNK2) Probable G-protein coupled receptor 133 precursor (G-prc | Q6QNK2 | 96468.2 | 7.32E−03 |
| 345 | Q6UXX5 (Q6UXX5) ITI-like protein (Inter-alpha (Globulin) inhibitor H5-like) | Q6UXX5 | 143097.8 | 6.30E−03 |
| 346 | Q6ZMV7 (Q6ZMV7) Hypothetical protein FLJ16641 | Q6ZMV7 | 45118.72 | 5.36E−03 |
| 347 | Q6ZMX7 (Q6ZMX7) Hypothetical protein FLJ16607 | Q6ZMX7 | 147809.7 | 1.23E−03 |
| 348 | Q6ZQN2 (Q6ZQN2) Hypothetical protein FLJ46846 | Q6ZQN2 | 180573.9 | 2.18E−03 |
| 349 | Q6ZVL2 (Q6ZVL2) Hypothetical protein FLJ42427 | Q6ZVL2 | 15702.14 | 7.92E−03 |
| 350 | Q6ZW87 (Q6ZW87) Hypothetical protein FLJ41443 | Q6ZW87 | 24773.63 | 5.09E−03 |
| 351 | Q7KYS7 (Q7KYS7) Hypothetical protein (Fragment) | Q7KYS7 | 31128.16 | 1.53E−03 |
| 352 | Q7RTS7 (Q7RTS7) Keratin 5c | Q7RTS7 | 59333.45 | 4.83E−03 |
| 353 | CA036_HUMAN (Q7Z3Z2) Protein C1orf36 | Q7Z3Z2 | 22689.57 | 1.27E−04 |
| 354 | Q7Z736 (Q7Z736) Hypothetical protein FLJ21019 | Q7Z736 | 85273.38 | 7.52E−03 |
| 355 | Q86VJ1 (Q86VJ1) E3 ligase for inhibin receptor | Q86VJ1 | 289427.4 | 9.51E−03 |
| 356 | Q86Y22 (Q86Y22) Alpha 1 type XXIII collagen | Q86Y22 | 51912.29 | 3.71E−03 |
| 357 | Q8IUA7 (Q8IUA7) ATP-binding cassette sub-family A member 9 | Q8IUA7 | 184241.6 | 5.35E−03 |
| 358 | Q8IUG8 (Q8IUG8) Carnitine transporter 2 | Q8IUG8 | 60830.3 | 6.69E−03 |
| 359 | Q8IUS2 (Q8IUS2) LOC339483 protein | Q8IUS2 | 2870.257 | 3.48E−03 |
| 360 | Q8IZA4 (Q8IZA4) ELYS transcription factor-like protein TMBS62 | Q8IZA4 | 256022.6 | 5.66E−03 |
| 361 | ASPM_HUMAN (Q8IZT6) Abnormal spindle-like microcephaly-associated protein (Abn | Q8IZT6 | 409539.8 | 4.72E−03 |
| 362 | Q8N1G4 (Q8N1G4) Hypothetical protein LRRC47 (Novel protein) | Q8N1G4 | 63433.89 | 2.19E−03 |
| 363 | Q8N401 (Q8N401) KIAA1632 protein | Q8N401 | 52340.19 | 8.67E−03 |
| 364 | DCAK2_HUMAN (Q8N568) Serine/threonine-protein kinase DCAMKL2 (EC 2.7.1.37) ( | Q8N568 | 83587.57 | 4.26E−03 |
| 365 | Q8N5E0 (Q8N5E0) Apoptosis-inducing factor like, isoform 2 | Q8N5E0 | 65924.3 | 5.12E−03 |
| 366 | Q8NC99 (Q8NC99) Hypothetical protein FLJ90396 | Q8NC99 | 66758.55 | 5.35E−03 |
| 367 | Q8NF06 (Q8NF06) FLJ00398 protein (Fragment) | Q8NF06 | 72000.41 | 1.78E−04 |
| 368 | Q8NFY8 (Q8NFY8) Neuroblastoma-amplified protein | Q8NFY8 | 268295.4 | 1.13E−03 |
| 369 | O10A7_HUMAN (Q8NGE5) Olfactory receptor 10A7 | Q8NGE5 | 35670.34 | 8.56E−03 |
| 370 | Q8NH29 (Q8NH29) Seven transmembrane helix receptor | Q8NH29 | 25373.85 | 1.70E−04 |
| 371 | IL17D_HUMAN (Q8TAD2) Interleukin-17D precursor (IL-17D) (Interleukin-27) (IL-2 | Q8TAD2 | 21879.14 | 6.37E−03 |
| 372 | Q8TCE1 (Q8TCE1) SERPINC1 protein | Q8TCE1 | 29073.79 | 2.30E−05 |
| 373 | Q8TCJ2 (Q8TCJ2) Source of immunodominant MHC-associated peptides | Q8TCJ2 | 93613.76 | 3.14E−04 |
| 374 | DDX54_HUMAN (Q8TDD1) DEAD-box protein 54 (EC 3.6.1.—) (ATP-dependent RNA h | Q8TDD1 | 98534.24 | 1.74E−03 |
| 375 | Q92772 (Q92772) P56 KKIAMRE protein kinase (Cyclin-dependent kinase-like 2) | Q92772 | 55982.93 | 6.77E−03 |
| 376 | COR2A_HUMAN (Q92828) Coronin-2A (WD repeat-containing protein 2) (IR10) | Q92828 | 59725.43 | 3.04E−03 |
| 377 | Q96E20 (Q96E20) CD99L2 protein (Hypothetical protein) (Fragment) | Q96E20 | 16612.17 | 3.63E−03 |
| 378 | Q96IV6 (Q96IV6) C5orf4 protein | Q96IV6 | 38976.07 | 4.48E−03 |
| 379 | RSRC1_HUMAN (Q96IZ7) Arginine/serine-rich coiled coil protein 1 | Q96IZ7 | 38654.46 | 8.17E−03 |
| 380 | KCNH8_HUMAN (Q96L42) Potassium voltage-gated channel subfamily H member 8 ( | Q96L42 | 123754.3 | 3.90E−04 |
| 381 | Q96MP4 (Q96MP4) Hypothetical protein FLJ32091 | Q96MP4 | 81413.94 | 6.67E−03 |
| 382 | Q96NI8 (Q96NI8) Hypothetical protein FLJ30791 | Q96NI8 | 62289.69 | 4.72E−03 |
| 383 | ZN285_HUMAN (Q96NJ3) Zinc finger protein 285 | Q96NJ3 | 50009.57 | 2.66E−03 |
| 384 | INP4A_HUMAN (Q96PE3) Type I inositol-3,4-bisphosphate 4-phosphatase (EC 3.1.3. | Q96PE3 | 109885.5 | 2.98E−04 |
| 385 | UHRF2_HUMAN (Q96PU4) Ubiquitin-like containing PHD and RING finger domains pr | Q96PU4 | 89927.86 | 6.47E−03 |
| 386 | Q96QE0 (Q96QE0) Hypothetical protein DL8Q12 | Q96QE0 | 23959.2 | 3.39E−03 |
| 387 | PANX2_HUMAN (Q96RD6) Pannexin-2 | Q96RD6 | 69434.98 | 7.29E−03 |
| 388 | Q96RF2 (Q96RF2) WWOXdelta5-8 | Q96RF2 | 35019.5 | 3.55E−03 |
| 389 | LYST_HUMAN (Q99698) Lysosomal trafficking regulator (Beige homolog) | Q99698 | 428865.6 | 7.47E−03 |
| 390 | SMO_HUMAN (Q99835) Smoothened homolog precursor (SMO) (Gx protein) | Q99835 | 86341.18 | 7.04E−05 |
| 391 | Q99993 (Q99993) WUGSC:H_2G3A.1 protein | Q99993 | 60051.64 | 4.36E−03 |
| 392 | OSR10_HUMAN (Q9BXB5) Oxysterol binding protein-related protein 10 (OSBP-related | Q9BXB5 | 83917.38 | 7.10E−03 |
| 393 | EMR3_HUMAN (Q9BY15) EGF-like module containing mucin-like hormone receptor-li | Q9BY15 | 72545.69 | 6.97E−03 |
| 394 | Q9BYJ0 (Q9BYJ0) Ksp37 (HBp17-related protein) | Q9BYJ0 | 24565.15 | 6.42E−04 |
| 395 | OSR7_HUMAN (Q9BZF2) Oxysterol binding protein-related protein 7 (OSBP-related | Q9BZF2 | 95371.7 | 3.33E−04 |
| 396 | Q9BZH2 (Q9BZH2) False p73 target protein | Q9BZH2 | 53043.29 | 2.66E−03 |
| 397 | PHAR1_HUMAN (Q9C0D0) Phosphatase and actin regulator 1 | Q9C0D0 | 54573.55 | 2.72E−04 |
| 398 | Q9C0D4 (Q9C0D4) KIAA1729 protein (Fragment) | Q9C0D4 | 113646.6 | 1.84E−03 |
| 399 | DDX24_HUMAN (Q9GZR7) ATP-dependent RNA helicase DDX24 (DEAD-box protein | Q9GZR7 | 96271.52 | 7.26E−03 |
| 400 | Q9GZT6 (Q9GZT6) MDS011 (Hypothetical protein MDS025) | Q9GZT6 | 29453.17 | 4.53E−03 |
| 401 | Q9H0J7 (Q9H0J7) Hypothetical protein DKFZp434F0116 | Q9H0J7 | 53323.07 | 2.81E−04 |
| 402 | GHITM_HUMAN (Q9H3K2) Growth hormone inducible transmembrane protein (Derm | Q9H3K2 | 37180.59 | 4.28E−03 |
| 403 | Q9H3M9 (Q9H3M9) Homo sapiens (Fragment) | Q9H3M9 | 39581.41 | 5.36E−03 |
| 404 | RANB3_HUMAN (Q9H6Z4) Ran-binding protein 3 (RanBP3) | Q9H6Z4 | 60173.32 | 3.82E−03 |
| 405 | SMYD3_HUMAN (Q9H7B4) SET and MYND domain-containing protein 3 (EC 2.1.1.43 | Q9H7B4 | 49050.59 | 5.56E−03 |
| 406 | Q9H7C4 (Q9H7C4) Hypothetical protein FLJ21054 | Q9H7C4 | 17715.02 | 8.68E−03 |
| 407 | COG4_HUMAN (Q9H9E3) Conserved oligomeric Golgi complex component 4 | Q9H9E3 | 89038.48 | 9.55E−03 |
| 408 | UBP29_HUMAN (Q9HBJ7) Ubiquitin carboxyl-terminal hydrolase 29 (EC 3.1.2.15) (U | Q9HBJ7 | 104090.1 | 1.22E−03 |
| 409 | Q9HBY0 (Q9HBY0) Putative superoxide-generating NADPH oxidase Mox2 (NADPH o | Q9HBY0 | 64893.13 | 4.96E−03 |
| 410 | SYTL2_HUMAN (Q9HCH5) Synaptotagmin-like protein 2 (Exophilin-4) | Q9HCH5 | 100734.4 | 8.61E−04 |
| 411 | Q9NR16 (Q9NR16) Scavenger receptor cysteine-rich type 1 protein M160 precursor | Q9NR16 | 159159.5 | 6.17E−03 |
| 412 | SPTN5_HUMAN (Q9NRC6) Spectrin beta chain, brain 4 (Spectrin, non-erythroid beta | Q9NRC6 | 416577.2 | 3.96E−04 |
| 413 | DC13_HUMAN (Q9NRP2) UPF0287 protein DC13 | Q9NRP2 | 9453.72 | 2.81E−03 |
| 414 | Q9NS87 (Q9NS87) Kinesin-like protein 2 | Q9NS87 | 160060.4 | 4.45E−03 |
| 415 | Q9NSN6 (Q9NSN6) Hypothetical protein DKFZp761O0113 | Q9NSN6 | 39970.1 | 6.56E−03 |
| 416 | EMIL3_HUMAN (Q9NT22) EMILIN-3 precursor (EMILIN-5) (Elastin microfibril interf | Q9NT22 | 82595.73 | 3.69E−03 |
| 417 | UGGG1_HUMAN (Q9NYU2) UDP-glucose:glycoprotein glucosyltransferase 1 precurs | Q9NYU2 | 174866.5 | 3.83E−03 |
| 418 | T2R14_HUMAN (Q9NYV8) Taste receptor type 2 member 14 (T2R14) (Taste receptor | Q9NYV8 | 36136.48 | 1.33E−03 |
| 419 | COMD9_HUMAN (Q9P000) COMM domain containing protein | Q9P000 | 21904.53 | 4.29E−04 |
| 420 | VPS18_HUMAN (Q9P253) Vacuolar protein sorting 18 (hVPS18) | Q9P253 | 110115.9 | 7.01E−03 |
| 421 | RRBP1_HUMAN (Q9P2E9) Ribosome-binding protein 1 (Ribosome receptor protein) ( | Q9P2E9 | 152380 | 4.24E−03 |
| 422 | SUCB1_HUMAN (Q9P2R7) Succinyl-CoA ligase [ADP-forming] beta-chain, mitochond | Q9P2R7 | 50299.33 | 8.47E−03 |
| 423 | GT2D1_HUMAN (Q9UHL9) General transcription factor II-I repeat domain-containin | Q9UHL9 | 105990.8 | 4.56E−03 |
| 424 | Q9UI74 (Q9UI74) PRO0245 | Q9UI74 | 8075.004 | 7.12E−04 |
| 425 | SPB13_HUMAN (Q9UIV8) Serpin B13 (Hurpin) (HaCaT UV-repressible serpin) (Protea | Q9UIV8 | 44248.2 | 8.72E−04 |
| 426 | HOOK1_HUMAN (Q9UJC3) Hook homolog 1 (h-hook1) (hHK1) | Q9UJC3 | 84594.84 | 9.77E−03 |
| 427 | DKKL1_HUMAN (Q9UK85) Dickkopf-like protein 1 precursor (Soggy-1 protein) (SGY-1 | Q9UK85 | 26990.23 | 1.36E−04 |
| 428 | PADI3_HUMAN (Q9ULW8) Protein-arginine deiminase type III (EC 3.5.3.15) (Peptid | Q9ULW8 | 74695.37 | 9.91E−04 |
| 429 | MLL4_HUMAN (Q9UMN6) Myeloid/lymphoid or mixed-lineage leukemia protein 4 (Trit | Q9UMN6 | 293327.3 | 9.43E−03 |
| 430 | GPR34_HUMAN (Q9UPC5) Probable G-protein coupled receptor 34 | Q9UPC5 | 43831.09 | 8.71E−03 |
| 431 | Q9UQ10 (Q9UQ10) Dimeric dihydrodiol dehydrogenase (EC 1.3.1.20) | Q9UQ10 | 36358.77 | 9.73E−03 |
| 432 | SHOC2_HUMAN (Q9UQ13) Leucine-rich repeat protein SHOC-2 (Ras-binding protein | Q9UQ13 | 64847.64 | 3.26E−03 |
| 433 | AT11B_HUMAN (Q9Y2G3) Probable phospholipid-transporting ATPase IF (EC 3.6.3.1 | Q9Y2G3 | 134104.1 | 9.84E−03 |
| 434 | PCDG7_HUMAN (Q9Y5G6) Protocadherin gamma A7 precursor (PCDH-gamma-A7) | Q9Y5G6 | 101659.2 | 2.96E−03 |
| 435 | PCDA2_HUMAN (Q9Y5H9) Protocadherin alpha 2 precursor (PCDH-alpha2) | Q9Y5H9 | 102000.4 | 6.26E−03 |
| 436 | Q9Y5S5 (Q9Y5S5) DNA polymerase epsilon catalytic subunit protein isoform a | Q9Y5S5 | 262846.1 | 9.10E−03 |
| 437 | SO1B1_HUMAN (Q9Y6L6) Solute carrier organic anion transporter family member 1B | Q9Y6L6 | 76398.98 | 1.51E−03 |

TABLE 11
Vitreous proteins identified from patients with retinal detachment (RD) & Macular Hole (MH)

| | | | | |
|---|---|---|---|---|
| 438 | Serotransferrin precursor | P02787 | 76999.66 | 4.22E−14 |
| 439 | Serum albumin precursor | P02768 | 69321.63 | 1.00E−30 |
| 440 | Hypothetical protein | Q569I7 | 20655.36 | 1.27E−07 |
| 441 | Transthyretin precursor | P02766 | 15877.05 | 1.00E−30 |
| 442 | Interphotoreceptor retinoid-binding protein precursor | P10745 | 135277.8 | 1.00E−30 |
| 443 | Keratin, type II cytoskeletal 1 | P04264 | 65846.88 | 9.37E−12 |
| 444 | Clusterin precursor | P10909 | 52461.05 | 3.33E−12 |
| 445 | Alpha-1-acid glycoprotein 1 precursor | P02763 | 23496.77 | 5.78E−12 |
| 446 | PH domain containing protein | Q8TD55 | 53317.19 | 3.94E−05 |
| 447 | Ig gamma-1 chain C region | P01857 | 36083.16 | 4.94E−13 |
| 448 | Apolipoprotein A-I precursor | P02647 | 30758.94 | 3.44E−14 |
| 449 | Hemopexin precursor | P02790 | 51643.32 | 8.88E−15 |
| 450 | Keratin, type I cytoskeletal 10 | P13645 | 59482.76 | 1.51E−09 |
| 451 | Transcription factor NF-E4 | Q86UQ8 | 19006.73 | 1.59E−04 |
| 452 | Apolipoprotein A-II precursor | P02652 | 11167.9 | 5.95E−12 |
| 453 | Keratin 10 | Q14664 | 57212.99 | 1.11E−15 |
| 454 | Hemoglobin delta subunit | P02042 | 15914.25 | 1.78E−14 |
| 455 | Pigment epithelium-derived factor precursor | P36955 | 46313.36 | 1.93E−10 |
| 456 | Alpha-1-antitrypsin precursor | P01009 | 46707.09 | 8.88E−15 |
| 457 | Alpha 2 globin variant | Q53F97 | 15270.94 | 1.89E−12 |
| 458 | Hypothetical protein FLJ42076 | Q6ZVU4 | 21036.72 | 8.88E−05 |
| 459 | Nuclear receptor-interacting factor | Q8WY14 | 52754.03 | 1.57E−03 |
| 460 | Keratin, type II cytoskeletal 1b | Q7Z794 | 61650.4 | 2.57E−06 |
| 461 | Hypothetical protein FLJ40259 | Q8N7W7 | 66086.88 | 1.29E−04 |
| 462 | Keratin, type II cytoskeletal | P35908 | 65825.37 | 2.77E−09 |
| 463 | Beta-globin gene from a thalassemia patient | Q14473 | 18918.59 | 2.17E−12 |
| 464 | HNRBF-2 | Q9H2I2 | 31787.43 | 5.30E−05 |
| 465 | Dickkopf-related protein 3 precursor | Q9UBP4 | 38266.07 | 2.33E−08 |
| 466 | Double-stranded RNA-binding protein | O95793 | 63227.92 | 1.39E−04 |
| 467 | Complement C4-A precursor | P0C0L4 | 192649.5 | 4.22E−14 |
| 468 | Hypothetical protein | Q6PHR9 | 9188.304 | 6.10E−04 |
| 469 | Vacuolar protein sorting 13A | Q96RL7 | 360046.1 | 9.97E−04 |
| 470 | Hypothetical protein DKFZp686I04196 | Q6N093 | 46031.66 | 1.25E−11 |
| 471 | Vitamin D-binding protein | Q6GTG1 | 52902.04 | 2.26E−10 |
| 472 | KIAA1200 protein | Q9ULM0 | 155607.6 | 1.07E−03 |
| 473 | Tumor necrosis factor | Q5VWH1 | 20099.21 | 1.04E−03 |
| 474 | PML-RARA regulated adaptor molecule | Q8N6W7 | 73950.18 | 1.05E−03 |
| 475 | Hypothetical protein DKFZp686J11235 | Q6MZW0 | 54424.95 | 4.12E−10 |
| 476 | Tuberin (Tuberous sclerosis 2 protein) | P49815 | 200621.4 | 7.82E−05 |
| 477 | Kallikrein-14 precursor | Q9P0G3 | 27434.89 | 1.67E−04 |
| 478 | K1C9_HUMAN (P35527) Keratin, type I cytoskeletal 9 (Cytokeratin-9) (CK-9) (Kera | P35527 | 62091.76 | 2.20E−12 |
| 479 | IGKC protein | Q502W4 | 25919.92 | 1.11E−15 |
| 480 | Zinc finger protein 185 | O15231 | 49156.87 | 4.35E−03 |
| 481 | IGLC2 protein | Q567P1 | 24784.13 | 1.61E−13 |
| 482 | Receptor-type tyrosine protein phosphatase delta | P23468 | 214623.5 | 2.34E−03 |
| 483 | ADAMTS-12 precursor | P58397 | 177429.5 | 2.30E−04 |
| 484 | Complement C3 precursor | P01024 | 187045.3 | 5.42E−11 |
| 485 | Adenylyl cyclase type V | Q7RTV7 | 39312.25 | 1.51E−04 |
| 486 | Hypothetical protein TMEM55A | Q8N4L2 | 28062.26 | 3.73E−05 |
| 487 | Cytochrome P450, family 20, subfamily A, | Q8WWA9 | 52460.08 | 2.11E−06 |
| 488 | Ceruloplasmin precursor | P00450 | 122127.6 | 1.11E−12 |
| 489 | Colony stimulating factor 2 receptor | Q4V312 | 46871.63 | 7.80E−05 |
| 490 | Hypothetical protein FLJ35435 | Q8NAF6 | 65308.66 | 1.80E−05 |
| 491 | Hypothetical protein FAM33A | Q8WVK7 | 14179.33 | 1.51E−03 |
| 492 | Alpha-2-macroglobulin precursor | P01023 | 163174.3 | 5.36E−09 |
| 493 | RhoGTPase regulating protein variant | Q6RJU1 | 130153.4 | 5.25E−04 |
| 494 | SGCE_HUMAN (O43556) Epsilon-sarcoglycan precursor (Epsilon-SG) | O43556 | 49722.25 | 2.18E−04 |
| 495 | Intercellular adhesion molecule 3 precursor | P32942 | 59345.85 | 2.21E−03 |
| 496 | Proto-oncogene tyrosine protein kinase | P07332 | 93412.31 | 4.63E−05 |
| 497 | Calcium-independent phospholipase A2 | Q9NP80 | 88421.11 | 2.83E−03 |
| 498 | WD-repeat protein 9 | Q9NSI6 | 257060.4 | 1.15E−03 |
| 499 | Ig gamma-3 chain C region | P01860 | 32309.81 | 2.03E−09 |
| 500 | Dystroglycan precursor | Q14118 | 97519.91 | 1.67E−03 |
| 501 | LOC150763 protein | Q6NUI2 | 87507.18 | 7.57E−05 |
| 502 | Osteopontin precursor | P10451 | 35401.25 | 5.20E−10 |
| 503 | Q6ZTG8 (Q6ZTG8) Hypothetical protein FLJ44670 | Q6ZTG8 | 115755 | 3.09E−03 |
| 504 | Apolipoprotein E precursor | P02649 | 36131.79 | 3.48E−08 |
| 505 | Novel protein | Q5T7N3 | 107275.7 | 1.60E−03 |
| 506 | Hypothetical protein FLJ40243 | Q7Z745 | 180566.4 | 1.50E−03 |
| 507 | Hypothetical protein DKFZp434I138 | Q659E3 | 78784.1 | 2.57E−04 |
| 508 | Hypothetical protein FLJ43007 | Q6ZV44 | 19332.49 | 1.02E−04 |
| 509 | Protein C19orf10 precursor (Stromal cell-derived GF) | Q969H8 | 18783.32 | 2.10E−03 |
| 510 | Q9NXK0 (Q9NXK0) Hypothetical protein FLJ20202 | Q9NXK0 | 44899.86 | 1.52E−03 |
| 511 | Basement membrane-specific heparan sulfate proteoglycan | P98160 | 468528.2 | 4.04E−04 |
| 512 | O95978 (O95978) VH1 protein precursor (Fragment) | O95978 | 17292.5 | 1.17E−07 |
| 513 | Alpha-2-HS-glycoprotein precursor | P02765 | 39299.73 | 1.29E−06 |
| 514 | Cytochrome P450 | P10635 | 55766.2 | 4.19E−04 |
| 515 | Zinc finger protein 197 | O14709 | 118770.8 | 4.48E−04 |
| 516 | Q8N7Q3 (Q8N7Q3) Hypothetical protein FLJ40479 | Q8N7Q3 | 45893.4 | 1.50E−03 |
| 517 | Trichohyalin | Q07283 | 247074.4 | 2.97E−03 |
| 518 | Hypothetical protein FLJ44832 | Q6ZQT3 | 139335.1 | 2.50E−03 |
| 519 | Hypothetical protein KIAA1505 | Q8IYE0 | 112734.9 | 2.15E−03 |
| 520 | G patch domain containing protein 3 | Q96I76 | 59301.11 | 2.28E−03 |
| 521 | ADAM 28 precursor | Q9UKQ2 | 87151.09 | 2.62E−04 |
| 522 | Beta-2-glycoprotein I precursor | P02749 | 38272.67 | 3.39E−08 |
| 523 | Alpha-internexin | Q16352 | 55357.48 | 6.89E−04 |
| 524 | Q684R2 (Q684R2) Para-hydroxybenzoate-polyprenyltransferase, mitochondrial (EC | Q684R2 | 45578.79 | 3.25E−03 |
| 525 | Q9Y2W9 (Q9Y2W9) Endocrine regulator | Q9Y2W9 | 231761.3 | 1.57E−04 |
| 526 | G protein-activated inward rectifier potassium channel 1 | P48549 | 56566.8 | 4.27E−04 |
| 527 | Keratin, type I cytoskeletal 12 | Q99456 | 53478.53 | 7.68E−06 |
| 528 | Gelsolin precursor | P06396 | 85644.25 | 2.02E−04 |
| 529 | TSP2_HUMAN (P35442) Thrombospondin-2 precursor | P35442 | 129871.5 | 3.33E−04 |
| 530 | Transmembrane channel-like protein 7 | Q7Z402 | 83447.49 | 2.53E−03 |
| 531 | Protein KIAA1199 precursor | Q8WUJ3 | 152900 | 1.11E−03 |
| 532 | Filamin-A | P21333 | 280583.4 | 1.64E−03 |
| 533 | Prostaglandin-H2 D-isomerase | P41222 | 21015.35 | 3.34E−10 |
| 534 | HSAL5836 | Q6UWG9 | 9682.161 | 2.94E−03 |
| 535 | Hypothetical protein gs80 | Q96S02 | 25483.99 | 9.47E−04 |
| 536 | AACT_HUMAN (P01011) Alpha-1-antichymotrypsin precursor (ACT) [Contains: Alpha- | P01011 | 47620.63 | 2.48E−04 |
| 537 | MRP1_HUMAN (P33527) Multidrug resistance-associated protein 1 (ATP-binding cas | P33527 | 171450.5 | 1.77E−03 |
| 538 | Keratin, type II cytoskeletal 2 | Q01546 | 65830.09 | 4.77E−04 |
| 539 | Maguin-like protein variant II | Q5SGD5 | 61848.88 | 1.71E−03 |
| 540 | Q6PHR8 (Q6PHR8) Hypothetical protein (Fragment) | Q6PHR8 | 33452.8 | 2.10E−03 |
| 541 | Q8NDD1 (Q8NDD1) Hypothetical protein DKFZp547B1713 | Q8NDD1 | 32732.49 | 5.52E−03 |
| 542 | Q9H6E6 (Q9H6E6) Hypothetical protein FLJ22346 | Q9H6E6 | 81825.66 | 1.41E−03 |
| 543 | Mitochondrial 28S ribosomal protein S2 | Q9Y399 | 33228.08 | 5.60E−05 |
| 544 | Supervillin | O95425 | 247550.6 | 5.54E−03 |
| 545 | RETBP_HUMAN (P02753) Plasma retinol-binding protein precursor (PRBP) (RBP) [C | P02753 | 22995.26 | 1.36E−03 |
| 546 | Ubiquitin conjugation factor E4 | Q14139 | 122482 | 7.16E−05 |
| 547 | Q9ULI2 (Q9ULI2) KIAA1238 protein (Fragment) | Q9ULI2 | 45141.04 | 2.40E−03 |
| 548 | HYES_HUMAN (P34913) Epoxide hydrolase 2 (EC 3.3.2.3) (Soluble epoxide hydrolas | P34913 | 62574.77 | 4.29E−04 |
| 549 | MYO7A_HUMAN (Q13402) Myosin-7A (Myosin VIIa) | Q13402 | 254242.5 | 8.23E−04 |
| 550 | PCSK5_HUMAN (Q92824) Proprotein convertase subtilisin/kexin type 5 precursor ( | Q92824 | 101708.3 | 2.56E−04 |
| 551 | TR10A_HUMAN (O00220) Tumor necrosis factor receptor superfamily member 10A p | O00220 | 50028.81 | 3.21E−04 |
| 552 | TBC1 domain family member 4 (Akt substrate) | O60343 | 146470.8 | 7.09E−04 |
| 553 | Dual specificity protein phosphatase 5 | Q16690 | 42080.39 | 2.53E−04 |
| 554 | TRPM8_HUMAN (Q7Z2W7) Transient receptor potential cation channel subfamily M | Q7Z2W7 | 127573.3 | 7.58E−04 |
| 555 | RUN domain containing 1 | Q8IXT9 | 67603.02 | 2.55E−03 |
| 556 | Q8NHQ3 (Q8NHQ3) RBBP8 protein | Q8NHQ3 | 102594.6 | 6.21E−05 |
| 557 | GCN1-like protein 1 (HsGCN1) | Q92616 | 292424.8 | 5.09E−03 |
| 558 | ZIC2_HUMAN (O95409) Zinc finger protein ZIC 2 (Zinc finger protein of the cere | O95409 | 54971.09 | 2.32E−03 |
| 559 | Q702P2 (Q702P2) VPS13C-2B protein | Q702P2 | 407940.5 | 4.75E−03 |
| 560 | MINT_HUMAN (Q96T58) Msx2-interacting protein (SPEN homolog) (SMART/HDAC1 | Q96T58 | 402004.3 | 2.49E−03 |
| 561 | COPG2_HUMAN (Q9UBF2) Coatomer gamma-2 subunit (Gamma-2 coat protein) (G | Q9UBF2 | 97559.69 | 1.54E−03 |
| 562 | CYTC_HUMAN (P01034) Cystatin C precursor (Neuroendocrine basic polypeptide) (Ga | P01034 | 15789.08 | 2.77E−05 |
| 563 | Q8TBE7 (Q8TBE7) TMEM22 protein | Q8TBE7 | 46419.88 | 7.86E−03 |
| 564 | K2C6A_HUMAN (P02538) Keratin, type II cytoskeletal 6A (Cytokeratin-6A) (CK 6A) | P02538 | 59877.28 | 7.41E−07 |
| 565 | VTNC_HUMAN (P04004) Vitronectin precursor (Serum spreading factor) (S-protein) | P04004 | 54271.23 | 5.97E−08 |
| 566 | OPT_HUMAN (Q9UBM4) Opticin precursor (Oculoglycan) | Q9UBM4 | 37237.39 | 4.68E−07 |
| 567 | Q15299 (Q15299) RARB protein | Q15299 | 11644 | 2.59E−04 |
| 568 | Q8N780 (Q8N780) Hypothetical protein FLJ25943 | Q8N780 | 33347.2 | 8.78E−04 |
| 569 | Q8N8Z1 (Q8N8Z1) Hypothetical protein FLJ38687 | Q8N8Z1 | 16623.52 | 1.10E−03 |
| 570 | IPO9_HUMAN (Q96P70) Importin-9 (Imp9) (Ran-binding protein 9) (RanbP9) | Q96P70 | 115757.8 | 7.94E−04 |
| 571 | SYP2L_HUMAN (Q9H987) Synaptopodin 2-like protein | Q9H987 | 102406.1 | 7.97E−03 |
| 572 | TLR5_HUMAN (O60602) Toll-like receptor 5 precursor (Toll/interleukin-1 receptor | O60602 | 97663.38 | 3.64E−03 |
| 573 | A1AG2_HUMAN (P19652) Alpha-1-acid glycoprotein 2 precursor (AGP 2) (Orosomuc | P19652 | 23587.64 | 4.46E−06 |
| 574 | PSME3_HUMAN (P61289) Proteasome activator complex subunit 3 (Proteasome activ | P61289 | 29487.55 | 3.25E−03 |
| 575 | ADIP_HUMAN (Q9Y2D8) Afadin- and alpha-actinin-binding protein (ADIP) (Afadin D | Q9Y2D8 | 71191.48 | 2.87E−03 |
| 576 | PDE6A_HUMAN (P16499) Rod cGMP-specific 3′,5′-cyclic phosphodiesterase alpha-su | P16499 | 99438.76 | 4.04E−03 |
| 577 | TCPQ_HUMAN (P50990) T-complex protein 1, theta subunit (TCP-1-theta) (CCT-thet | P50990 | 59451.57 | 2.31E−03 |
| 578 | Q7KZ97 (Q7KZ97) Antithrombin III variant | Q7KZ97 | 52657.96 | 3.10E−06 |
| 579 | Q86WI3 (Q86WI3) NOD27 | Q86WI3 | 204535 | 2.47E−03 |
| 580 | Q969T7 (Q969T7) Hypothetical protein MGC20781 | Q969T7 | 33551.02 | 5.37E−03 |
| 581 | DGC14_HUMAN (Q96DF8) DGCR14 protein (DiGeorge syndrome critical region 14) ( | Q96DF8 | 52535.89 | 5.80E−04 |
| 582 | Q96LU7 (Q96LU7) Hypothetical protein FLJ25056 | Q96LU7 | 31208.16 | 1.08E−03 |
| 583 | PCDA7_HUMAN (Q9UN72) Protocadherin alpha 7 precursor (PCDH-alpha7) | Q9UN72 | 100802.9 | 3.78E−03 |
| 584 | RHG26_HUMAN (Q9UNA1) Rho-GTPase-activating protein 26 (Oligophrenin-1-like pr | Q9UNA1 | 92176.67 | 4.55E−03 |
| 585 | TF2AY_HUMAN (Q9UNN4) TFIIA-alpha and beta-like factor (General transcription f | Q9UNN4 | 52412.98 | 4.50E−03 |

TABLE 12
Proteins identified in both Retinal Detachment and Macular Hole diagnosed patients (10 patients, 50 runs)
SEQUEST filters: p prob < 0.01; Xcorr vs charge state: 1.5 for [M + H]1+, 2.0 for [M + 2H]2+, 2.5 for [M + 3H]3+; DCn > 0.1

| | | | | | | | [symbol] tide | [symbol] s in 10 | in 50 |
|---|---|---|---|---|---|---|---|---|---|
| | Pathology | Patients | Reference | P (pro) | MW | Accession | (Hits) | paids | runs |
| 1 | RD_MH | GFILAEBD | Serotransferrin precursor | 4.22E−14 | 76999.66 | P02787 | 20 | 10 | 50 |
| 2 | RD_MH | GILHBACE | Serum albumin precursor | 1.00E−30 | 69321.63 | P02768 | 11 | 10 | 50 |
| 3 | RD_MH | LGHIACDE | Hypothetical protein | 1.27E−07 | 20655.36 | Q56917 | 3 | 7 | 46 |
| 4 | RD_MH | LlFGECBA | Transthyretin precursor | 1.00E−30 | 15877.05 | P02766 | 4 | 10 | 43 |
| 5 | RD_MH | BDACHILF | Interphotoreceptor retinoid-binding protein precur [symbol] | 1.00E−30 | 135277.8 | P10745 | 1 | 10 | 43 |
| 6 | RD_MH | [symbol] LGIHC- | Keratin, type II cytoskeletal 1 | 9.37E−12 | 65846.88 | P04264 | 7 | 6 | 41 |
| | | BD [symbol] | | | | | | | |
| 7 | RD_MH | BDECLFIG | Clusterin precursor | 3.33E−12 | 52461.05 | P10909 | 9 | 10 | 40 |
| 8 | RD_MH | FGLIDAEC | Alpha-1-acid glycoprotein 1 precursor | 5.78E−12 | 23496.77 | P02763 | 2 | 6 | 32 |
| 9 | RD_MH | [symbol] GIHCA- | PH domain containing protein | 3.94E−05 | 53317.19 | Q8TD55 | 1 | 5 | 32 |
| | | DE [symbol] | | | | | | | |
| 10 | RD_MH | BCDEFHG | Ig gamma-1 chain C region | 4.94E−13 | 36083.16 | P01857 | 8 | 7 | 30 |
| 11 | RD_MH | FGIHAEBD | Apolipoprotein A-I precursor | 3.44E−14 | 30758.94 | P02647 | 9 | 5 | 28 |
| 12 | RD_MH | EBDCFLIH | Hemopexin precursor | 8.88E−15 | 51643.32 | P02790 | 4 | 6 | 27 |
| 13 | RD_MH | DBALGFH [symbol] | Keratin, type I cytoskeletal 10 | 1.51E−09 | 59482.76 | P13645 | 1 | 4 | 26 |
| 14 | RD_MH | [symbol] GLHCDEA | Transcription factor NF-E4 | 1.59E−04 | 19006.73 | Q86UQ8 | 1 | 6 | 25 |
| 15 | RD_MH | [symbol] EABFGH [symbol] | Apolipoprotein A-II precursor | 5.95E−12 | 11167.9 | P02652 | 1 | 5 | 25 |
| 16 | RD_MH | [symbol] FIHGB- | Keratin 10 | 1.11E−15 | 57212.99 | Q14664 | 1 | 6 | 24 |
| | | CD [symbol] | | | | | | | |
| 17 | RD_MH | BDFHI | Hemoglobin delta subunit | 1.78E−14 | 15914.25 | P02042 | 13 | 3 | 24 |
| 18 | RD_MH | BEALHGI [symbol] | Pigment epithelium-derived factor precursor | 1.93E−10 | 46313.36 | P36955 | 8 | 4 | 22 |
| 19 | RD_MH | [symbol] ECDBFG [symbol] | Alpha-1-antitrypsin precursor | 8.88E−15 | 46707.09 | P01009 | 4 | 7 | 21 |
| 20 | RD_MH | FHGIDBC | Alpha 2 globin variant | 1.89E−12 | 15270.94 | Q53F97 | 5 | 6 | 21 |
| 21 | RD_MH | [symbol] ILHGA- | Hypothetical protein FLJ42076 | 8.88E−05 | 21036.72 | Q6ZVU4 | 1 | 6 | 21 |
| | | EC [symbol] | | | | | | | |
| 22 | RD_MH | [symbol] LGIAE- | Nuclear receptor-interacting factor | 1.57E−03 | 52754.03 | Q8WY14 | 1 | 5 | 21 |
| | | CB [symbol] | | | | | | | |
| 23 | RD_MH | DAECHLIG | Keratin, type II cytoskeletal 1b | 2.57E−06 | 61650.4 | Q7Z794 | 2 | 9 | 20 |
| 24 | RD_MH | ADCEGHI [symbol] | Hypothetical protein FLJ40259 | 1.29E−04 | 66086.88 | Q8N7W7 | 1 | 5 | 19 |
| 25 | RD_MH | HGLFBA | Keratin, type II cytoskeletal | 2.77E−09 | 65825.37 | P35908 | 1 | 9 | 17 |
| 26 | RD_MH | BDFIH | Beta-globin gene from a thalassemia patient | 2.17E−12 | 18918.59 | Q14473 | 10 | 6 | 17 |
| 27 | RD_MH | ECDAGIH [symbol] | HNRBF-2 | 5.30E−05 | 31787.43 | Q9H2I2 | 1 | 6 | 15 |
| 28 | RD_MH | ABHFLI | Dickkopf-related protein 3 precursor | 2.33E−08 | 38266.07 | Q9UBP4 | 3 | 5 | 15 |
| 29 | RD_MH | DECALHG | Double-stranded RNA-binding protein | 1.39E−04 | 63227.92 | O95793 | 1 | 5 | 14 |
| 30 | RD_MH | ABEDGHF | Complement C4-A precursor | 4.22E−14 | 192649.5 | P0C0L4 | 1 | 6 | 13 |
| 31 | RD_MH | IGLHCD | Hypothetical protein | 6.10E−04 | 9188.304 | Q6PHR9 | 1 | 6 | 13 |
| 32 | RD_MH | BCDGLI | Vacuolar protein sorting 13A | 9.97E−04 | 360046.1 | Q96RL7 | 1 | 5 | 13 |
| 33 | RD_MH | ABFLIH | Hypothetical protein DKFZp686I04196 | 1.25E−11 | 46031.66 | Q6N093 | 6 | 3 | 13 |
| 34 | RD_MH | FHGAE | Vitamin D-binding protein | 2.26E−10 | 52902.04 | Q6GTG1 | 1 | 7 | 12 |
| 35 | RD_MH | ILHEDCA | KIAA1200 protein | 1.07E−03 | 155607.6 | Q9ULM0 | 2 | 4 | 12 |
| 36 | RD_MH | ECAHFIL | Tumor necrosis factor | 1.04E−03 | 20099.21 | Q5VWH1 | 1 | 4 | 12 |
| 37 | RD_MH | [symbol] GLDEA [symbol] | PML-RARA regulated adaptor molecule | 1.05E−03 | 73950.18 | Q8N6W7 | 1 | 4 | 12 |
| 38 | RD_MH | FGDBCA | Hypothetical protein DKFZp686J11235 | 4.12E−10 | 54424.95 | Q6MZW0 | 4 | 6 | 11 |
| 39 | RD_MH | ACEILFG | Tuberin (Tuberous sclerosis 2 protein) | 7.82E−05 | 200621.4 | P49815 | 1 | 4 | 11 |
| 40 | RD_MH | CAEGHL | Kallikrein-14 precursor | 1.67E−04 | 27434.89 | Q9P0G3 | 1 | 4 | 11 |
| 41 | RD_MH | BGLFH | K1C9_HUMAN (P35527) Keratin, type I cytoskele | 2.20E−12 | 62091.76 | P35527 | 2 | 3 | 11 |
| 42 | RD_MH | FHGIA | IGKC protein | 1.11E−15 | 25919.92 | Q502W4 | 3 | 7 | 10 |
| 43 | RD_MH | AEDCBGI | Zinc finger protein 185 | 4.35E−03 | 49156.87 | O15231 | 1 | 8 | 9 |
| 44 | RD_MH | FHBA | IGLC2 protein | 1.61E−13 | 24784.13 | Q567P1 | 3 | 7 | 9 |
| 45 | RD_MH | FIGEC | Receptor-type tyrosine-protein phosphatase delta | 2.34E−03 | 214623.5 | P23468 | 1 | 6 | 9 |
| 46 | RD_MH | CDAEH | ADAMTS-12 precursor | 2.30E−04 | 177429.5 | P58397 | 1 | 5 | 9 |
| 47 | RD_MH | ABDFG | Complement C3 precursor | 5.42E−11 | 187045.3 | P01024 | 1 | 4 | 9 |
| 48 | RD_MH | GHLB | Adenylyl cyclase type V | 1.51E−04 | 39312.25 | Q7RTV7 | 1 | 4 | 9 |
| 49 | RD_MH | HLIGBD | Hypothetical protein TMEM55A | 3.73E−05 | 28062.26 | Q8N4L2 | 1 | 9 | 8 |
| 50 | RD_MH | HILD | Cytochrome P450, family 20, subfamily A, | 2.11E−06 | 52460.08 | Q8WWA9 | 1 | 8 | 8 |
| 51 | RD_MH | BAEF | Ceruloplasmin precursor | 1.11E−12 | 122127.6 | P00450 | 3 | 6 | 8 |
| 52 | RD_MH | EDCBG | Colony stimulating factor 2 receptor | 7.80E−05 | 46871.63 | Q4V312 | 1 | 5 | 8 |
| 53 | RD_MH | IGHACB | Hypothetical protein FLJ35435 | 1.80E−05 | 65308.66 | Q8NAF6 | 1 | 5 | 8 |
| 54 | RD_MH | GHILAC | Hypothetical protein FAM33A | 1.51E−03 | 14179.33 | Q8WVK7 | 1 | 5 | 8 |
| 55 | RD_MH | LBA | Alpha-2-macroglobulin precursor | 5.36E−09 | 163174.3 | P01023 | 2 | 3 | 8 |
| 56 | RD_MH | HFAB | RhoGTPase regulating protein variant | 5.25E−04 | 130153.4 | Q6RJU1 | 1 | 3 | 8 |
| 57 | RD_MH | AHIG | SGCE_HUMAN (O43556) Epsilon-sarcoglycan p [symbol] | 2.18E−04 | 49722.25 | O43556 | 1 | 2 | 8 |
| 58 | RD_MH | GHLABE | Intercellular adhesion molecule 3 precursor | 2.21E−03 | 59345.85 | P32942 | 1 | 6 | 7 |
| 59 | RD_MH | ECHLG | Proto-oncogene tyrosine-protein kinase | 4.63E−05 | 93412.31 | P07332 | 1 | 5 | 7 |
| 60 | RD_MH | ECGIL | Calcium-independent phospholipase A2 | 2.83E−03 | 88421.11 | Q9NP80 | 1 | 5 | 7 |
| 61 | RD_MH | HLGDCB | WD-repeat protein 9 | 1.15E−03 | 257060.4 | Q9NSI6 | 1 | 5 | 7 |
| 62 | RD_MH | FBA | Ig gamma-3 chain C region | 2.03E−09 | 32309.81 | P01860 | 1 | 4 | 7 |
| 63 | RD_MH | ILEAD | Dystroglycan precursor | 1.67E−03 | 97519.91 | Q14118 | 2 | 3 | 7 |
| 64 | RD_MH | CAF | LOC150763 protein | 7.57E−05 | 87507.18 | Q6NUI2 | 1 | 3 | 7 |
| 65 | RD_MH | LAB | Osteopontin precursor | 5.20E−10 | 35401.25 | P10451 | 1 | 2 | 7 |
| 66 | RD_MH | GEC | Q6ZTG8 (Q6ZTG8) Hypothetical protein FLJ4467 | 3.09E−03 | 115755 | Q6ZTG8 | 1 | 2 | 7 |
| 67 | RD_MH | LHFB | Apolipoprotein E precursor | 3.48E−08 | 36131.79 | P02649 | 1 | 8 | 6 |
| 68 | RD_MH | LGHE | Novel protein | 1.60E−03 | 107275.7 | Q5T7N3 | 1 | 7 | 6 |
| 69 | RD_MH | BCEIH | Hypothetical protein FLJ40243 | 1.50E−03 | 180566.4 | Q7Z745 | 1 | 7 | 6 |
| 70 | RD_MH | EDCBH | Hypothetical protein DKFZp434I138 | 2.57E−04 | 78784.1 | Q659E3 | 1 | 6 | 6 |
| 71 | RD_MH | EDG | Hypothetical protein FLJ43007 | 1.02E−04 | 19332.49 | Q6ZV44 | 1 | 6 | 6 |
| 72 | RD_MH | HIGAD | Protein C19orf10 precursor (Stromal cell-derived | 2.10E−03 | 18783.32 | Q969H8 | 1 | 4 | 6 |
| 73 | RD_MH | GCE | Q9NXK0 (Q9NXK0) Hypothetical protein FLJ2020 | 1.52E−03 | 44899.86 | Q9NXK0 | 1 | 4 | 6 |
| 74 | RD_MH | CEGH | Basement membrane-specific heparan sulfate pro [symbol] | 4.04E−04 | 468528.2 | P98160 | 1 | 3 | 6 |
| 75 | RD_MH | AF | O95978 (O95978) VH1 protein precursor (Fragm [symbol] | 1.17E−07 | 17292.5 | O95978 | 1 | 2 | 6 |
| 76 | RD_MH | FGA | Alpha-2-HS-glycoprotein precursor | 1.29E−06 | 39299.73 | P02765 | 2 | 7 | 5 |
| 77 | RD_MH | AEF | Cytochrome P450 | 4.19E−04 | 55766.2 | P10635 | 1 | 7 | 5 |
| 78 | RD_MH | HLIAB | Zinc finger protein 197 | 4.48E−04 | 118770.8 | O14709 | 1 | 5 | 5 |
| 79 | RD_MH | FBCA | Q8N7Q3 (Q8N7Q3) Hypothetical protein FLJ4047 | 1.50E−03 | 45893.4 | Q8N7Q3 | 1 | 5 | 5 |
| 80 | RD_MH | CAHG | Trichohyalin | 2.97E−03 | 247074.4 | Q07283 | 1 | 4 | 5 |
| 81 | RD_MH | IHBD | Hypothetical protein FLJ44832 | 2.50E−03 | 139335.1 | Q6ZQT3 | 1 | 4 | 5 |
| 82 | RD_MH | ILE | Hypothetical protein KIAA1505 | 2.15E−03 | 112734.9 | Q8IYE0 | 1 | 4 | 5 |
| 83 | RD_MH | CDEI | G patch domain containing protein 3 | 2.28E−03 | 59301.11 | Q96I76 | 1 | 4 | 5 |
| 84 | RD_MH | CDHLI | ADAM 28 precursor | 2.62E−04 | 87151.09 | Q9UKQ2 | 1 | 4 | 5 |
| 85 | RD_MH | FB | Beta-2-glycoprotein I precursor | 3.39E−08 | 38272.67 | P02749 | 1 | 3 | 5 |
| 86 | RD_MH | GHADC | Alpha-internexin | 6.89E−04 | 55357.48 | Q16352 | 1 | 3 | 5 |
| 87 | RD_MH | CLH | Q684R2 (Q684R2) Para-hydroxybenzoate--polyp [symbol] | 3.25E−03 | 45578.79 | Q684R2 | 1 | 3 | 5 |
| 88 | RD_MH | LGDE | Q9Y2W9 (Q9Y2W9) Endocrine regulator | 1.57E−04 | 231761.3 | Q9Y2W9 | 1 | 3 | 5 |
| 89 | RD_MH | EDHL | G protein-activated inward rectifier potassium cha | 4.27E−04 | 56566.8 | P48549 | 1 | 6 | 4 |
| 90 | RD_MH | LIB | Keratin, type I cytoskeletal 12 | 7.68E−06 | 53478.53 | Q99456 | 1 | 6 | 4 |
| 91 | RD_MH | FLB | Gelsolin precursor | 2.02E−04 | 85644.25 | P06396 | 1 | 5 | 4 |
| 92 | RD_MH | AFIG | TSP2_HUMAN (P35442) Thrombospondin-2 pre [symbol] | 3.33E−04 | 129871.5 | P35442 | 1 | 5 | 4 |
| 93 | RD_MH | ECAH | Transmembrane channel-like protein 7 | 2.53E−03 | 83447.49 | Q7Z402 | 1 | 5 | 4 |
| 94 | RD_MH | BADF | Protein KIAA1199 precursor | 1.11E−03 | 152900 | Q8WUJ3 | 1 | 5 | 4 |
| 95 | RD_MH | HGDE | Filamin-A | 1.64E−03 | 280583.4 | P21333 | 1 | 4 | 4 |
| 96 | RD_MH | ABF | Prostaglandin-H2 D-isomerase | 3.34E−10 | 21015.35 | P41222 | 1 | 4 | 4 |
| 97 | RD_MH | FIE | HSAL5836 | 2.94E−03 | 9682.161 | Q6UWG9 | 1 | 4 | 4 |
| 98 | RD_MH | DBHL | Hypothetical protein gs80 | 9.47E−04 | 25483.99 | Q96S02 | 1 | 4 | 4 |
| 99 | RD_MH | BHL | AACT_HUMAN (P01011) Alpha-1-antichymotryps [symbol] | 2.48E−04 | 47620.63 | P01011 | 1 | 3 | 4 |
| 100 | RD_MH | AGIF | MRP1 HUMAN (P33527) Multidrug resistance-as | 1.77E−03 | 171450.5 | P33527 | 1 | 3 | 4 |
| 101 | RD_MH | FB | Keratin, type II cytoskeletal 2 | 4.77E−04 | 65830.09 | Q01546 | 1 | 3 | 4 |
| 102 | RD_MH | DEGF | Maguin-like protein variant II | 1.71E−03 | 61848.88 | Q5SGD5 | 1 | 3 | 4 |
| 103 | RD_MH | LB | Q6PHR8 (Q6PHR8) Hypothetical protein (Fragme [symbol] | 2.10E−03 | 33452.8 | Q6PHR8 | 1 | 3 | 4 |
| 104 | RD_MH | LGED | Q8NDD1 (Q8NDD1) Hypothetical protein DKFZp5 | 5.52E−03 | 32732.49 | Q8NDD1 | 1 | 3 | 4 |
| 105 | RD_MH | FCA | Q9H6E6 (Q9H6E6) Hypothetical protein FLJ2234 | 1.41E−03 | 81825.66 | Q9H6E6 | 1 | 2 | 4 |
| 106 | RD_MH | ACI | Mitochondrial 28S ribosomal protein S2 | 5.60E−05 | 33228.08 | Q9Y399 | 1 | 2 | 4 |
| 107 | RD_MH | BAL | Supervillin | 5.54E−03 | 247550.6 | O95425 | 1 | 6 | 3 |
| 108 | RD_MH | AFG | RETBP_HUMAN (P02753) Plasma retinol-bindin [symbol] | 1.36E−03 | 22995.26 | P02753 | 1 | 6 | 3 |
| 109 | RD_MH | GHA | Ubiquitin conjugation factor E4 | 7.16E−05 | 122482 | Q14139 | 1 | 5 | 3 |
| 110 | RD_MH | HEB | Q9ULI2 (Q9ULI2) KIAA1238 protein (Fragment) | 2.40E−03 | 45141.04 | Q9ULI2 | 1 | 5 | 3 |
| 111 | RD_MH | LEA | HYES_HUMAN (P34913) Epoxide hydrolase 2 (E | 4.29E−04 | 62574.77 | P34913 | 1 | 4 | 3 |
| 112 | RD_MH | ELH | MYO7A_HUMAN (Q13402) Myosin-7A (Myosin V | 8.23E−04 | 254242.5 | Q13402 | 1 | 4 | 3 |
| 113 | RD_MH | LIA | PCSK5_HUMAN (Q92824) Proprotein convertas [symbol] | 2.56E−04 | 101708.3 | Q92824 | 1 | 4 | 3 |
| 114 | RD_MH | IAD | TR10A_HUMAN (O00220) Tumor necrosis factor | 3.21E−04 | 50028.81 | O00220 | 1 | 3 | 3 |
| 115 | RD_MH | DAF | TBC1 domain family member 4 (Akt substrate) | 7.09E−04 | 146470.8 | O60343 | 1 | 3 | 3 |
| 116 | RD_MH | BAH | Dual specificity protein phosphatase 5 | 2.53E−04 | 42080.39 | Q16690 | 1 | 3 | 3 |
| 117 | RD_MH | GHA | TRPM8_HUMAN (Q7Z2W7) Transient receptor p | 7.58E−04 | 127573.3 | Q7Z2W7 | 1 | 3 | 3 |
| 118 | RD_MH | CEI | RUN domain containing 1 | 2.55E−03 | 67603.02 | Q8IXT9 | 1 | 3 | 3 |
| 119 | RD_MH | LHB | Q8NHQ3 (Q8NHQ3) RBBP8 protein | 6.21E−05 | 102594.6 | Q8NHQ3 | 1 | 3 | 3 |
| 120 | RD_MH | CEF | GCN1-like protein 1 (HsGCN1) | 5.09E−03 | 292424.8 | Q92616 | 1 | 3 | 3 |
| 121 | RD_MH | LEC | ZIC2_HUMAN (O95409) Zinc finger protein ZIC 2 | 2.32E−03 | 54971.09 | O95409 | 1 | 2 | 3 |
| 122 | RD_MH | FE | Q702P2 (Q702P2) VPS13C-2B protein | 4.75E−03 | 407940.5 | Q702P2 | 1 | 2 | 3 |
| 123 | RD_MH | CLH | MINT_HUMAN (Q96T58) Msx2-interacting protei [symbol] | 2.49E−03 | 402004.3 | Q96T58 | 1 | 2 | 3 |
| 124 | RD_MH | FA | COPG2_HUMAN (Q9UBF2) Coatomer gamma-2 | 1.54E−03 | 97559.69 | Q9UBF2 | 1 | 2 | 3 |
| 125 | RD_MH | HA | CYTC_HUMAN (P01034) Cystatin C precursor ( [symbol] | 2.77E−05 | 15789.08 | P01034 | 1 | 6 | 2 |
| 126 | RD_MH | HB | Q8TBE7 (Q8TBE7) TMEM22 protein | 7.86E−03 | 46419.88 | Q8TBE7 | 1 | 6 | 2 |
| 127 | RD_MH | LB | K2C6A_HUMAN (P02538) Keratin, type II cytoske | 7.41E−07 | 59877.28 | P02538 | 1 | 5 | 2 |
| 128 | RD_MH | FB | VTNC_HUMAN (P04004) Vitronectin precursor ( [symbol] | 5.97E−08 | 54271.23 | P04004 | 1 | 5 | 2 |
| 129 | RD_MH | BL | OPT_HUMAN (Q9UBM4) Opticin precursor (Ocu [symbol] | 4.68E−07 | 37237.39 | Q9UBM4 | 1 | 5 | 2 |
| 130 | RD_MH | HE | Q15299 (Q15299) RARB protein | 2.59E−04 | 11644 | Q15299 | 1 | 4 | 2 |
| 131 | RD_MH | EI | Q8N780 (Q8N780) Hypothetical protein FLJ2594 [symbol] | 8.78E−04 | 33347.2 | Q8N780 | 1 | 4 | 2 |
| 132 | RD_MH | CH | Q8N8Z1 (Q8N8Z1) Hypothetical protein FLJ3868 | 1.10E−03 | 16623.52 | Q8N8Z1 | 1 | 4 | 2 |
| 133 | RD_MH | EG | IPO9_HUMAN (Q96P70) Importin-9 (Imp9) (Ran- | 7.94E−04 | 115757.8 | Q96P70 | 1 | 4 | 2 |
| 134 | RD_MH | EG | SYP2L_HUMAN (Q9H987) Synaptopodin 2-like p | 7.97E−03 | 102406.1 | Q9H987 | 1 | 4 | 2 |
| 135 | RD_MH | EI | TLR5_HUMAN (O60602) Toll-like receptor 5 prec | 3.64E−03 | 97663.38 | O60602 | 2 | 3 | 2 |
| 136 | RD_MH | BF | A1AG2_HUMAN (P19652) Alpha-1-acid glycopro [symbol] | 4.46E−06 | 23587.64 | P19652 | 1 | 3 | 2 |
| 137 | RD_MH | AH | PSME3_HUMAN (P61289) Proteasome activator | 3.25E−03 | 29487.55 | P61289 | 1 | 3 | 2 |
| 138 | RD_MH | FC | ADIP_HUMAN (Q9Y2D8) Afadin- and alpha-actin | 2.87E−03 | 71191.48 | Q9Y2D8 | 1 | 3 | 2 |
| 139 | RD_MH | CH | PDE6A_HUMAN (P16499) Rod cGMP-specific 3′ | 4.04E−03 | 99438.76 | P16499 | 1 | 2 | 2 |
| 140 | RD_MH | AI | TCPQ_HUMAN (P50990) T-complex protein 1, th | 2.31E−03 | 59451.57 | P50990 | 1 | 2 | 2 |
| 141 | RD_MH | FC | Q7KZ97 (Q7KZ97) Antithrombin III variant | 3.10E−06 | 52657.96 | Q7KZ97 | 1 | 2 | 2 |
| 142 | RD_MH | GE | Q86WI3 (Q86WI3) NOD27 | 2.47E−03 | 204535 | Q86WI3 | 1 | 2 | 2 |
| 143 | RD_MH | AG | Q969T7 (Q969T7) Hypothetical protein MGC2078 | 5.37E−03 | 33551.02 | Q969T7 | 1 | 2 | 2 |
| 144 | RD_MH | EG | DGC14_HUMAN (Q96DF8) DGCR14 protein (Di [symbol] | 5.80E−04 | 52535.89 | Q96DF8 | 1 | 2 | 2 |
| 145 | RD_MH | AH | Q96LU7 (Q96LU7) Hypothetical protein FLJ2505 [symbol] | 1.08E−03 | 31208.16 | Q96LU7 | 1 | 2 | 2 |
| 146 | RD_MH | IE | PCDA7_HUMAN (Q9UN72) Protocadherin alpha | 3.78E−03 | 100802.9 | Q9UN72 | 1 | 2 | 2 |
| 147 | RD_MH | FE | RHG26_HUMAN (Q9UNA1) Rho-GTPase-activat | 4.55E−03 | 92176.67 | Q9UNA1 | 1 | 2 | 2 |
| 148 | RD_MH | HA | TF2AY_HUMAN (Q9UNN4) TFIIA-alpha and beta | 4.50E−03 | 52412.98 | Q9UNN4 | 1 | 2 | 2 |

TABLE 13
Function and sub-cellular location of proteins identified in

| | | |
|---|---|---|
| | Sub-cellular | |
| Protein | Location | Function |

(A) Macular Hole

| | | |
|---|---|---|
| Sortilin-related receptor precursor | membrane | endocytic receptor |
| Neuronal membrane glycoprotein M6-a | membrane | neural development |
| Kinesin-like protein KIF1A | synaptic vesicles | axonal transport |
| Voltage-dependent T-type calcium channel alpha-1G subunit | membrane | calcium influx/eflux |
| Ryanodine receptor 2 (Cardiac muscle-type ryanodine receptor) | membrane | transverse-tubules and |
| | | sarcoplasmic reticulum |
| SOX-12 protein (SOX-22 protein) | nucleus | non-histone DNA binding |
| Frizzled 6 precursor (Frizzled-6) (Fz-6) (hFz6) | membrane | cell differentitation/morphogenesis |
| Retinoblastoma-binding protein 5 (RBBP-5) | nucleus | transcription |
| Retinoic acid receptor alpha (RAR-alpha) | nucleus | retonic acid receptor |

(B) Retinal Detachment

| | | |
|---|---|---|
| LOC131076 protein | unknown | function unknown, coiled coiled domain protein |
| T-cell activation Rho GTPase-activating protein | cytoplasm | GTP-ase activator, T cell activation |
| Huntingtin interacting protein E | cytoplasm | signal transduction |
| Synaptotagmin-5 (Synaptotagmin V) (SytV) | Synaptic | Calcium dependent exocytosis |
| | vesicle | |
| Huntingtin-associated protein-interacting protein | cytoplasm | signal transduction |
| VGFR2_Vascular endothelial growth factor receptor 2 precursor | membrane | angiogenesis, vascular permeability |
| Presynaptic protein SAP97 (Synapse-associated protein 97) | cytoskeleton | scaffolding protein |
| Retinoblastoma-associated factor 600 (RBAF600) (Fragment) | nucleus | possible chromatin remodeling |

1. A method of characterizing the physiological state of the eye, comprising detecting the presence or absence in vitreous fluid of one or more polypeptides or fragments thereof.

2. A method of characterizing the physiological state of the eye, comprising detecting the presence or absence in vitreous fluid of one or more biomarker attractant-associated polypeptides or fragments thereof.

3. A method of claim 1, wherein said method is to diagnose a disease.

4. A method of claim 1, wherein said method is to determine the risk of a disease.

5. A method of claim 1, wherein at least one said polypeptides or fragment is selected from the polypeptides listed in Table 2 to Table 13.

6. A method of claim 1, wherein said method is for characterizing the integrity of retinal blood vessels, comprising detecting the presence or absence in vitreous fluid of at least one polypeptide or fragment.

7. A method of characterizing the physiological state of a living system, comprising detecting the presence or absence in a vitreous fluid of one or more polypeptides or fragments thereof.

8. A method of claim 7, wherein said polypeptide is associated with a biomarker-attractant molecule.

9. A method of claim 7, for monitoring drug efficacy or dynamics.

10. A method of claim 7, which is a method for monitoring or characterizing the physiological state of the brain.

11. A method of claim 7, wherein at least one said polypeptide or fragment is selected from the polypeptides listed in Table 2 to Table 13.

12. A method of claim 7, which is a method for monitoring the physiological effects of intraocular or systemic drugs.

13. A method of monitoring the signaling pathway status of a subject, comprising:
measuring the presence of a phosphorylated polypeptide fragment in vitreous fluid.

14. A method of claim 13, wherein the phosphorylated polypeptide is a receptor polypeptide or a fragment thereof.

15. A method of claim 14, wherein the phosphorylated receptor is a G-protein coupled receptor or a hormone activated receptor.

16. A method of claim 14, wherein the phosphorylated receptor is VEGFR-2 or PDGFR-beta.

17. A method of monitoring the efficacy of a tyrosine kinase inhibitor in a subject to whom said inhibitor has been administered, comprising
measuring the presence of a phosphorylated polypeptide or fragment in a vitreous fluid of the subject, wherein the subject has been administered a tyrosine kinase inhibitor.

18. A method of claim 17, wherein said tyrosine kinase inhibitor inhibits the phosphorylation of a tyrosine-kinase receptor or enzyme.

19. A method of claim 1, wherein the detecting is accomplished using a protein micro array, immunoassay, ligand binding assay, electrophoresis, or mass spectroscopy of a vitreous fluid sample.

20. A method of claim 1, wherein the detecting is accomplished using non-invasive optical measurements of vitreous fluid samples.

21. A method of claim 1, wherein said sample is extracted into a reservoir comprising at least one chemical to protect the polypeptide integrity.

22. A method of claim 21, wherein the chemical is a protease inhibitor or a phosphatase inhibitor.

23. A hollow bore cannula or cutter for vitrectomy, comprising a reservoir in said cannula or cutter comprising at least one chemical to protect polypeptide integrity.

24. A proteomic fingerprint, comprising at least one, or a plurality of, isolated vitreous fluid polypeptides or fragments thereof or a measurement related thereto, wherein said polypeptides are selected from the polypeptides listed in Table 2 to Table 13.

25. A proteomic fingerprint of vitreous fluid, comprising at least one isolated vitreous fluid biomarker attractant-associated polypeptide or a fragment thereof or a measurement related thereto.

26. A proteomic fingerprint of claim 25, comprising a plurality of isolated vitreous fluid biomarker attractant-associated polypeptides or fragments thereof or a measurement related thereto.

27. A method of claim 3 wherein said disease is a retinal or ocular disease.

28. A method of claim 4 wherein said disease is a retinal or ocular disease.

29. A method of claim 6 wherein said polypeptide or fragment is not normally found in vitreous fluid but is normally found in blood.

30. A method of claim 3 wherein said disease is retinal detachment (RD) or macular hole (MH) comprising detecting the presence of one or more polypeptides of Table 11, Table 12 or Table 13.

31. A method of claim 3 wherein said disease is retinal detachment (RD) comprising detecting the presence of one or more polypeptides of Table 7 or Table 9.

32. A method of claim 3 wherein said disease is macular hole (MH) comprising detecting the presence of one or more polypeptides of Table 8 or Table 10.

33. A method of determining the association of a given disease and polypeptides present in said animal comprising comparing the spectrum of polypeptides in the vitreous fluid of a patient having the disease to the spectrum of polypeptides in the vitreous fluid of normal subject, determining the differences in said polypeptide spectra in terms of identity or amount of said polypeptide, and correlating the presence of said disease with at least one of said differences.

34. A Method of claim 33 in which the findings of the measurement are used to guide therapy using a drug, a biologic agent, a chemical, an antibody, autologous protein, or synthetic agent.
